(19) United States
(12) Patent Application Publication
(10) Pub. No.: US 2026/0116910 A1
(43) Pub. Date: Apr. 30, 2026

(54) POLYCYCLIC COMPOUNDS AND METHODS THEREOF

(71) Applicant: HEPAITECH (BEIJING) BIOPHARMA TECHNOLOGY CO., LTD., Beijing (CN)

(72) Inventors: Xiaoguang Lei, Beijing (CN); Xiaoming Wang, Beijing (CN); Yulong Li, Beijing (CN)

(21) Appl. No.: 18/710,074

(22) PCT Filed: Nov. 14, 2022
(86) PCT No.: PCT/CN2022/131653

§ 371 Date: May 14, 2024

(30) Foreign Application Priority Data
Nov. 15, 2021 (WO) PCT/CN2021/130774
Jan. 20, 2022 (WO) PCT/CN2022/072911

(51) Int. Cl. C07J 41/00; A61K 31/575; C07J 43/00

(52) U.S. Cl. CPC C07J 41/0061; A61K 31/575; C07J 43/003

(57) ABSTRACT

Among other things, the present disclosure provides technologies, e.g., compounds, compositions, methods, use for preventing or treating various conditions, disorders or diseases. In some embodiments, a condition, disorder or disease is nonalcoholic steatohepatitis.

### CROSS-REFERENCE TO RELATED APPLICATIONS

This application claims priority to PCT Application Nos. PCT/CN2021/130774, filed Nov. 15, 2021, and PCT/CN2022/072911, filed Jan. 20, 2022, the entirety of each of which is incorporated herein by reference.

### TECHNICAL FIELD

Among other things, the present disclosure provides technologies, e.g., compounds, compositions, methods, etc. that are useful, e.g., for treating various conditions, disorders or diseases.

### BACKGROUND

It has been reported that certain polycyclic compounds, e.g., bile acids and derivatives thereof, can in some instances provide biological activities. In some embodiments, such compounds are reported to be useful for treating certain conditions, disorders or diseases such as primary biliary cholangitis (PBC) in certain patient populations.

### SUMMARY

In some embodiments, the present disclosure provides technologies, e.g., compounds, compositions, methods, etc., that can provide various properties and activities. For example, in some embodiments, the present disclosure provides compounds of formula I or pharmaceutically acceptable salts thereof. In some embodiments, provided compounds can modulate an activity of Farnesoid X receptor (FXR). In some embodiments, provided compounds can modulate an activity of TGR5. In some embodiments, provided compounds can modulate an activity of a bile acid receptor.

Among other things, the present disclosure encompasses the recognition that various side effects associated with present uses (e.g., treatment of conditions, disorders or diseases) of certain compounds, e.g., bile acids and analogs or derivatives thereof (which typically comprise a polycyclic ring system found in a bile acid), are associated with their off-target effects, e.g., in some embodiment, undesired activation of MRGPRX4. For example, in some embodiments, FXR and/or TGR5 agonists such as bile acids and analogs or derivatives thereof can activate other polypeptides, e.g., MRGPRX4. In some embodiments, provided technologies can provide improved selectivity for their desired activities, e.g., activation of FXR and/or TGR5, over potential off-target effects, e.g., in some embodiments activation of MRGPRX4. In some embodiments, provided technologies can provide improved selectivity for activation of FXR over MRGPRX4. In some embodiments, provided technologies can provide improved selectivity for activation of TGR5 over MRGPRX4. In some embodiments, a reference technology is or comprises a compound of a natural bile acid. In some embodiments, a reference technology is or comprises a bile acid or an analog or derivative thereof which has 3-OH. In some embodiments, a reference technology is or comprises a compound which is otherwise identical with a provided compound but has 3-OH. In some embodiments, 3-OH has the stereochemistry as in a bile acid.

Particularly, in some embodiments, the present disclosure recognizes and demonstrates that removal of a hydroxyl group at C3 (e.g., by replacing it with —H) of a polycyclic ring system in bile acids (moiety A, below) can effectively reduce or remove off-target binding and/or side effects (e.g., undesired activation of MRGPRX4) of various bile acids and analogs and derivatives thereof.

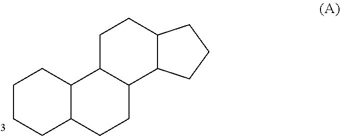 [SMILES: C1CCC2C(C1)CCC1C3CCCC3CCC21]

In some embodiments, compounds comprising moiety A above and can activate FXR is referred to as 3-OH bile acid compounds. Various such 3-OH bile acid compounds have been reported, and many technologies are available for assessing their activities including their activation of FXR, TGR5, and/or MRGPRX4. In some embodiments, a 3-OH bile acid compound is utilized as a reference compound for assessing activities and/or selectivity (e.g., for FXR and/or TGR5 over MRGPRX4) of a provided compound, e.g., a compound of formula I or a salt thereof. In some embodiments, a provided compound, e.g., a compound of formula I or a salt thereof, provides higher or comparable level of activation of a desired target, e.g., FXR or TGR5. In some embodiments, a provided compound, e.g., a compound of formula I or a salt thereof, provides higher selectivity for a desired target (e.g., FXR and/or TGR5) over another polypeptide (e.g., MRGPRX4). In some embodiments, a 3-OH bile acid compound is obeticholic acid or a salt thereof. In some embodiments, a 3-OH bile acid compound is cholic acid or a salt thereof.

In some embodiments, the present disclosure provides technologies that have improved potency, improved selectivity, improved therapeutic index and/or window, improved administration, improved regimen, improved biological, therapeutic and/or clinical outcome, reduced off-target effects, and/or reduced side effects compared to a comparable reference technology. In some embodiments, the present disclosure provides a method for increasing selectivity of a compound comprising a 3-OH group attached to moiety A, which selectivity is for modulation (e.g., activation) of a first polypeptide over a second polypeptide, comprising removing such a 3-OH group. In some embodiments, the present disclosure provides a method for increasing selectivity of a compound comprising a 3-OH group attached to moiety A, which selectivity is for modulation (e.g., activation) of a first polypeptide over a second polypeptide, comprising administering to a system a compound that does not contain such a 3-OH group but is otherwise identical or a pharmaceutically acceptable salt thereof. In some embodiments, a first polypeptide is or comprises FXR. In some embodiments, a first polypeptide is or comprises TGR5. In some embodiments, a second polypeptide is or comprises MRGPRX4. In some embodiments, a system is or comprises a cell, tissue, organ or organism. In some embodiments, a system is a cell. In some embodiments, a system is a subject. In some embodiments, a subject is a human. In some embodiments, a system is an in vitro system, e.g., a system suitable for in vitro assessment of activity and/or selectivity of a compound of present disclosure. In some embodiments, a system comprises or expresses a first polypeptide. In some embodiments, a system comprises or expresses a second polypeptide. In some embodiments, a system comprises or expresses a first and a second polypeptides. In some embodiments, a system comprises or expresses FXR. In some embodiments, a system comprises or expresses TGR5. In some embodiments, a system comprises or expresses MRGPRX4. In some embodiments, a system comprises or expresses FXR and MRGPRX4. In some embodiments, a system comprises or expresses TGR5 and MRGPRX4.

In some embodiments, the present disclosure provides methods for reducing an off-target effect and/or a side effect of a comparable technology which comprises a 3-OH group attached to moiety A comprising removing such a 3-OH group. In some embodiments, the present disclosure provides methods for reducing an off-target effect and/or a side effect associated with administration to a system of a compound comprising a 3-OH group attached to moiety A, comprising administering to the system a compound that does not contain a 3-OH group attached to moiety A. In some embodiments, the present disclosure provides methods for reducing an off-target effect and/or a side effect associated with administration to a system of a compound comprising a 3-OH group attached to moiety A, comprising administering to the system a compound that does not contain a 3-OH group attached to moiety A but is otherwise identical or a salt thereof. In some embodiments, the present disclosure provides methods for reducing an off-target effect and/or a side effect associated with administration to a system of a compound comprising a 3-OH group attached to moiety A, comprising administering to a system a compound that does not contain such a 3-OH group but is otherwise identical or a pharmaceutically acceptable salt thereof. In some embodiments, the present disclosure provides methods for reducing an off-target effect and/or a side effect associated with administration to a subject of a compound comprising a 3-OH group attached to moiety A, comprising administering to the subject a compound that does not contain a 3-OH group attached to moiety A but is otherwise identical or a salt thereof. In some embodiments, the present disclosure provides methods for reducing an off-target effect and/or a side effect associated with administration to a subject of a compound comprising a 3-OH group attached to moiety A, comprising administering to a subject a compound that does not contain such a 3-OH group but is otherwise identical or a pharmaceutically acceptable salt thereof. In some embodiments, the present disclosure provides methods for reducing an adverse reaction associated with administration to a subject of a compound comprising a 3-OH group attached to moiety A, comprising administering to the subject a compound that does not contain a 3-OH group attached to moiety A but is otherwise identical or a salt thereof. In some embodiments, the present disclosure provides methods for reducing an adverse reaction associated with administration to a subject of a compound comprising a 3-OH group attached to moiety A, comprising administering to a subject a compound that does not contain such a 3-OH group but is otherwise identical or a pharmaceutically acceptable salt thereof. In some embodiments, a compound comprising a 3-OH group attached to moiety A is a 3-OH bile acid compound as described herein. As described herein, removal of such a 3-OH group can be achieved through its replacement with various groups such as —H, halogen, etc. In some embodiments, removal of such a 3-OH group provides a compound of formula I or a salt thereof. In some embodiments, a compound comprising a 3-OH group attached to moiety A is a 3-OH bile acid compound. In some embodiments, a compound that does not contain such a 3-OH group but is otherwise identical or a pharmaceutically acceptable salt thereof is a compound of formula I or a salt thereof. In some embodiments, a compound that does not contain a 3-OH group attached to moiety A but is otherwise identical is administered in place of a compound comprising a 3-OH group attached to moiety A. In some embodiments, a compound comprising a 3-OH group attached to moiety A is administered at a reduced level. Those skilled in the art will appreciate that a compound that does not contain a 3-OH group attached to moiety A may be administered at the same or a different dose or regimen. In some embodiments, a side effect is an adverse reaction. In some embodiments, an off-target effect is or comprises activation of MRGPRX4. In some embodiments, a side effect or an adverse reaction is associated with MRGPRX4 activation.

In some embodiments, a side effect is or comprises an adverse reaction reported for cholic acid. In some embodiments, a side effect is or comprises an adverse reaction reported for obeticholic acid. In some embodiments, a side effect is or comprises hepatotoxicity. For example, in some embodiments, a side effect is or comprises exacerbation of liver impairment. In some embodiments, a side effect is or comprises diarrhea. In some embodiments, a side effect is or comprises hepatic decompensation and/or failure. In some embodiments, a side effect is fatal or results in liver transplant. In some embodiments, a side effect is in patients with cirrhosis. In some embodiments, a side effect is in patients with decompensated cirrhosis. In some embodiments, a side effect is or comprises pruritus. In some embodiments, a side effect is or comprises severe pruritus. In some embodiments, severe pruritus is or comprises intense or widespread itching, interfering with activities of daily living, or causing severe sleep disturbance, or intolerable discomfort, and typically requiring medical interventions. In some embodiments, a side effect is or comprises itching. In some embodiments, itching is intense. In some embodiments, itching is widespread. In some embodiments, itching is intense and widespread. In some embodiments, itching interferes with various activities, e.g., activities of daily living. In some embodiments, itching causes sleep disturbance. In some embodiments, itching causes intolerable discomfort. In some embodiments, a side effect, e.g., itching, severe pruritus, etc. requires medical interventions. In some embodiments, a side effect is one that typically leads to medical intervention. In some embodiments, a side effect is reduction of high-density lipoprotein-cholesterol (HDL-C) below a normal level. In some embodiments, a side effect is hepatic decompensation and/or failure. In some embodiments, a side effect is hepatic decompensation and failure in primary biliary cholangitis (PBC) patients with cirrhosis. In some embodiments, provided technologies reduce occurrence of one or more adverse reaction reported for cholic acid (e.g., exacerbation of liver impairment). In some embodiments, provided technologies reduce occurrence of one or more adverse reaction reported for obeticholic acid (e.g., hepatic decompensation and failure in PBC patients with cirrhosis, severe pruritus and/or reduction in HDL-C).

As appreciated by those skilled in the art, compounds of the present disclosure can be utilized for many purposes including for preventing or treating various conditions, disorders or diseases. In some embodiments, the present disclosure provides a method for treating a condition, disorder or disease, comprising administering or delivering to a subject suffering therefrom an effective amount of a provided compound, e.g., a FXR or TGR5 agonist comprising moiety A but no —OH at position 3 or a salt thereof, a compound of formula I or a pharmaceutically acceptable salt thereof, etc. In some embodiments, the present disclosure provides a method for preventing a condition, disorder or disease, comprising administering or delivering to a subject susceptible thereto an effective amount of a provided compound, e.g., a compound of formula I or a pharmaceutically acceptable salt thereof. In some embodiments, a condition, disorder or disease is reported to benefit from administration of a 3-OH bile acid compound. In some embodiments, a condition, disorder or disease is nonalcoholic steatohepatitis (NASH). In some embodiments, a condition, disorder or disease is a bile acid synthesis condition, disorder or disease. In some embodiments, a bile acid synthesis condition, disorder or disease is due to single enzyme defects (SEDs). In some embodiments, a compound may be utilized as an adjunctive treatment of a peroxisomal condition, disorder or disease, e.g., a Zellweger spectrum disorder. In some embodiments, a patient of a peroxisomal condition, disorder or disease, e.g., a Zellweger spectrum disorder, exhibit manifestations of a liver condition, disorder or disease, steatorrhea or complications from decreased fat-soluble vitamin absorption.

In some embodiments, the present disclosure provides pharmaceutical compositions which comprises or delivers compounds of present disclosure, e.g., compounds of formula I or pharmaceutically acceptable salts thereof, and pharmaceutically acceptable carriers. Various technologies are reported and can be utilized to manufacture pharmaceutical compositions of compounds of the present disclosure in accordance with the present disclosure. Typically, when utilized for preventing or treating conditions, disorders or diseases, compounds of the present disclosure are provided and administered as pharmaceutical compositions.

As appreciated by those skilled in the art, various technologies, e.g., reactions, reagents, conditions, etc. are useful for manufacturing provided compounds and compositions in accordance with the present disclosure. Certain such technologies are described below including in the Examples.

In some embodiments, the present disclosure provides technologies for assessing and characterizing provided compounds and compositions. Those skilled in the art reading the present disclosure will appreciate many technologies, including those described in the Examples, can be utilized to assess various properties and activities of compounds of the present disclosure, e.g., potency for modulating (e.g., activating) activities of polypeptides (e.g., FXR, TGR5, MRGPRX4, etc.), selectivity (e.g., for modulation (e.g., activation) of a first polypeptide (e.g., FXR, TGR5, etc.) over a second polypeptide (e.g., MRGPRX4)), etc.

As appreciated by those skilled in the art, compounds of the present disclosure may be provided in various forms, e.g., salts, esters, solvates, prodrugs, etc. In some embodiments, a provided compound is in a salt form. In some embodiments, a provided compound is a pharmaceutically acceptable salt form. In some embodiments, a provided compound is in a solvate form. In some embodiments, a provided compound is a prodrug. In some embodiments, a provided compound is an ester.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As appreciated by those skilled in the art, compounds may be provided, administered, or delivered in various forms, e.g., salts (e.g., pharmaceutically acceptable salts), solvates, hydrates, esters, prodrugs, tautomers, etc.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a bile acid analog differs structurally from a bile acid but performs at least one function of a bile acid (e.g., activation of FXR). In some embodiments, an analog comprises a characteristic structural feature of a reference chemical moiety. In some embodiments, a bile acid analog comprises moiety A. In some embodiments, a bile acid analog comprises moiety A and an acid group (e.g., —COOH) or a bioisostere thereof.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of amino acids, in some embodiments, a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 t electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, =NNHC(O)R*, =NNHC(O)OR*, =NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of Rf are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises two or more acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethvl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzvloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyrrolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethvl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, o-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxvmethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl, 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a protecting group is attached to a sulfur atom of a phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In some embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

As those skilled in the art will appreciate, methods and compositions described herein relating to provided compounds (e.g., oligonucleotides) generally also apply to pharmaceutically acceptable salts of such compounds.

### DESCRIPTION OF CERTAIN EMBODIMENTS

Among other things, the present disclosure provides compounds and compositions and methods thereof that are useful for preventing or treating various conditions, disorders or diseases. In some embodiments, compounds of the present disclosure are FXR agonists. In some embodiments, compounds of the present disclosure are TGR5 agonists. In some embodiments, compounds of the present disclosure do not activate MRGPRX4 when they activate FXR and/or TGR5 activities. Certain embodiments of provided technologies are described below as examples.

### Certain Embodiments of Compounds

In some embodiments, provided compounds are bile acids or analogs or derivatives thereof and do not have 3-OH, e.g., those described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804 and Yu et al. eLife 2019; 8:e48431, etc. 3-OH attached to moiety A replaced with R¹ or R^{1a} as described herein, in some embodiments, replaced with hydrogen.

In some embodiments, a provided compound is a compound of formula I:

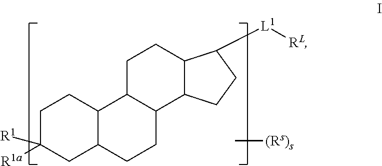

or a salt thereof, wherein:
   each of R¹ and R^{1a} is independently R^{s};
   each R^{s} is independently —H, -L″-R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, —N(R′)₂, a protected hydroxyl group, or R^{s} is

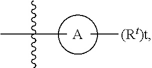

or two R^{s} attached to the same atom are taken together to form ═O or ═NR^{x};
   t is 0-6;
   each R^{t} is independently R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, or —N(R′)₂;
   each Ring A is independently an optionally substituted 3-20 membered monocyclic, bicyclic or polycyclic ring having 0-10 heteroatoms;
   each L″ is independently a covalent bond, or an optionally substituted, bivalent C₁₋₆ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   R^{L} is R^{s}, —C(O)R^{s}, —C(O)OR^{s}, —C(O)N(R^{s})₂, —C(O)N(R′)S(O)₂R^{s}, —C(O)N(R′)C(R′)₂C(O)N(R^{s})₂, —C(O)N(R′)C(R′)₂S(O)₂R^{s}, —C(O)N(R′)C(R′)₂S(O)₂N(R^{s})₂, —C(O)N(R′)C(R′)₂P(O)(R^{s})₂, —C(O)N(R′)C(R′)₂N(R′)C(O)N(R′)S(O)₂R^{s}, —C(O)N(R′)C(R^{s})₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂OR^{s}, —N(R^{s})₂, —N(R′)C(O)R^{s}, —N(R′)C(S)R^{s}, —N(R′)C(NR′)R^{s}, —N(R′)C(O)OR^{s}, —N(R′)C(O)N(R^{s})₂, —N(R′)C(NR′)N(R^{s})₂, —N(R′)C(O)N(R′)S(O)₂R^{s}, —N(R′)C(S)N(R′)S(O)₂R^{s}, —N(R′)C(NR′)N(R′)S(O)₂R^{s}, —N(R′)C(O)C(O)N(R′)S(O)₂R^{s}, —N(R′)C(O)N(R′)S(O)₂N(R^{s})₂, —N(R′)S(O)₂R^{s}, —OC(O)N(R^{s})₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s};
   s is 0-25;
   R^{x} is -L-R′, —Si(R′)₃, or a hydroxyl protecting group;
   L¹ is L;
   each L is independently a covalent bond, or an optionally substituted, bivalent C₁₋₁₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   each -Cy- is independently an optionally substituted bivalent, 3-20 membered, monocyclic, bicyclic or polycyclic ring having 0-10 heteroatoms;
   R′ is R, —OR, —C(O)R, —C(O)OR, —C(O)N(R)₂, or —S(O)₂R; and each R is independently —H, or an optionally substituted group selected from C₁₋₁₅ aliphatic, C₁₋₁₅ heteroaliphatic having 1-5 heteroatoms, C₆₋₁₄ aryl, C₆₋₁₄ aryl-C₁₋₁₅ aliphatic, C₆₋₁₄ aryl-C₁₋₁₅ heteroaliphatic having 1-5 heteroatoms, C₁₋₁₅ aliphatic-C₆₋₁₄ aryl, C₁₋₁₅ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₅ aliphatic, 5-14 membered heteroaryl having 1-10 heteroatoms-C₁₋₁₅ heteroaliphatic having 1-5 heteroatoms, C₁₋₁₅ aliphatic-5-14 membered heteroaryl having 1-10 heteroatoms, C₁₋₁₅ heteroaliphatic having 1-5 heteroatoms-5-14 membered heteroaryl having 1-10 heteroatoms, C₂₋₂₀ biaryl having 0-10 heteroatoms, and 3-20 membered heterocyclyl having 1-5 heteroatoms, or
   two R groups are optionally and independently taken together to form a covalent bond or ═O, or:
   two or more R groups on the same atom are optionally and independently taken together with the atom to form an optionally substituted, 3-20 membered, monocyclic, bicyclic or polycyclic ring having, in addition to the atom, 0-5 heteroatoms; or
   two or more R groups on two or more atoms are optionally and independently taken together with their intervening atoms to form an optionally substituted, 3-20 membered, monocyclic, bicyclic or polycyclic ring having, in addition to the intervening atoms, 0-5 heteroatoms.

In some embodiments, a provided compound, e.g., a compound of formula I, is a compound of formula II:

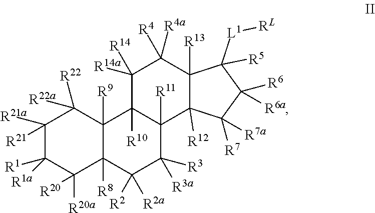

or a salt thereof, wherein:
   each of R¹, R^{1a}, R², R^{2a}, R³, R^{3a}, R⁴, R^{4a}, R⁵, R⁶, R^{6a}, R⁷, R^{7a}, R⁸, R⁹, R¹⁰, R¹¹, R¹², R¹³, R¹⁴, R^{14a}, R²⁰, R^{20a}, R²¹, R^{21a}, R²² and R^{22a} is independently R^{s}; and
   each other variable is independently as described herein.

In some embodiments, a compound has the structure of formula II-a:

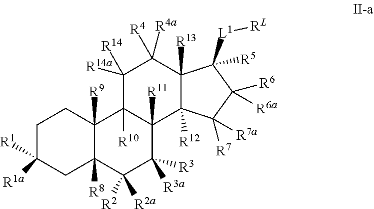

or a salt thereof, wherein each variable is independently as described herein.

In some embodiments, a compound has the structure of formula II-b:

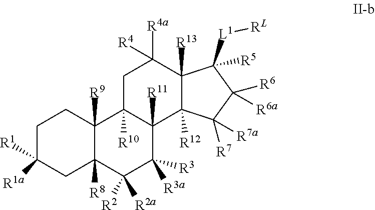

or a salt thereof, wherein each variable is independently as described herein.

In some embodiments, a compound has the structure of formula II-c, II-d, II-e, TI-f, II-g, II-h, II-i, II-j, II-k, II-l, II-m, II-n, II-o, II-p, II-q, II-r or II-s:

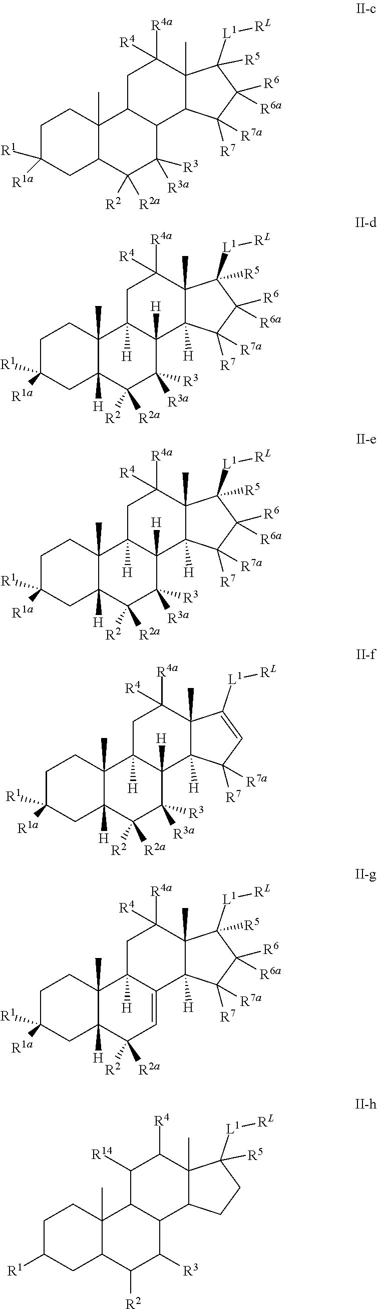

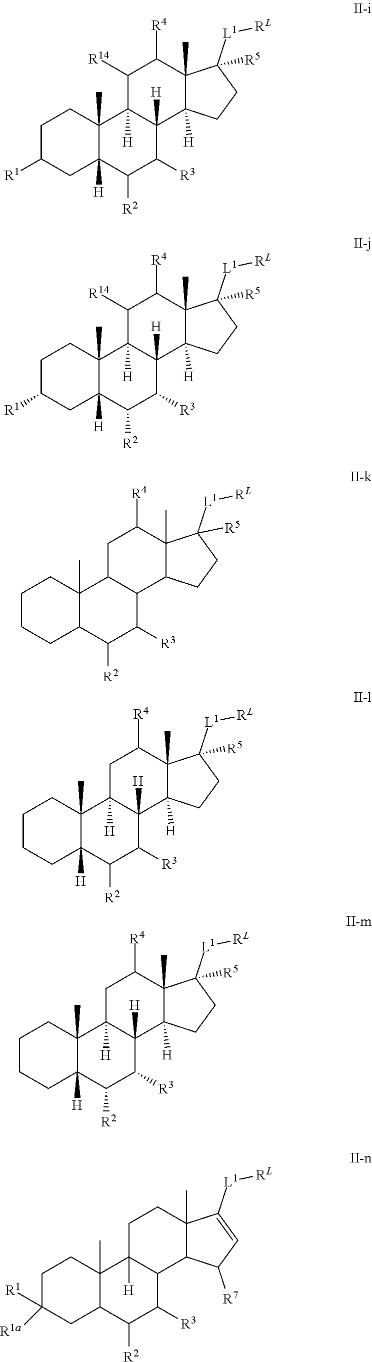

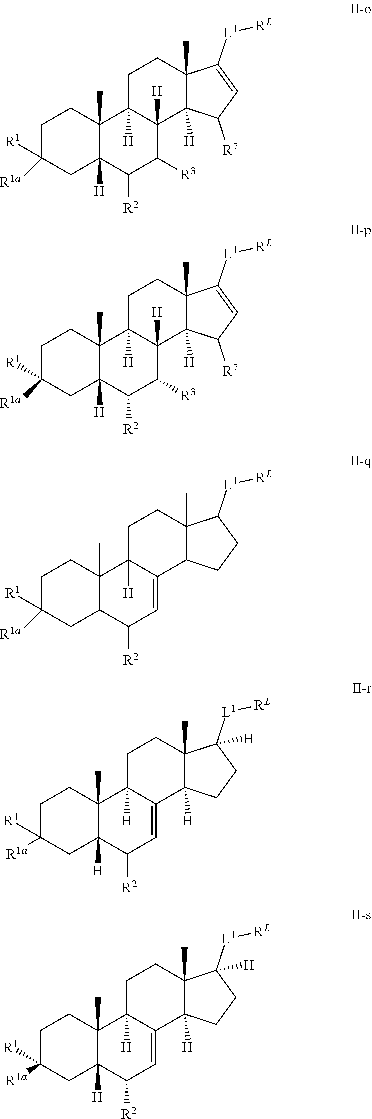

or a salt thereof, wherein each variable is independently as described herein.

Certain embodiments for various variables are described below as examples. Those skilled in the art reading the present disclosure will readily appreciate that embodiments for various variables can be combined in accordance with the present disclosure. Some combinations are described below as examples. In some embodiments, embodiments of a variable (e.g., R) are described when describing embodiments for other variables (e.g., various R embodiments are described when describing certain embodiments of R¹, R^{1a}, etc.). Those skilled in the art reading the present disclosure readily appreciate that embodiments of a variable described when describing any one variable (e.g., R embodiments when describing R¹) may be applied to other variables that can be this variable (e.g., R^{1a}, R², etc. which can be R).

### R^{1a}

In some embodiments, R^{1a} is R^{s}. In some embodiments, R^{1a} is —H. In some embodiments, R^{1a} is halogen. In some embodiments, R^{1a} is —F. In some embodiments, R^{1a} is —Cl. In some embodiments, R^{1a} is —Br. In some embodiments, R^{1a} is —CN. In some embodiments, R^{1a} is —N₃.

In some embodiments, R^{1a} is —C(O)R′, —S(O)₂R′, —OS(O)₂R′, —OP(O)(R′)₂, —SR′, or —N(R′)₂, wherein each R′ is independently as described herein.

In some embodiments, R^{1a} is —OR. In some embodiments, R^{1a} is —OR, wherein R is not hydrogen. In some embodiments, R^{1a} is —OH. In some embodiments, R^{1a} is —OC(O)R. In some embodiments, R is —H. In some embodiments, R is optionally substituted C₁₋₆ aliphatic.

In some embodiments, R^{1a} is —S(O)₂OR wherein R is as described herein. In some embodiments, R^{1a} is —S(O)₂OH. In some embodiments, R^{1a} is in a salt form, e.g., a sodium salt form.

In some embodiments, R^{1a} is a protected hydroxyl group. In some embodiments, R^{1a} is —OTBS.

In some embodiments, R^{1a} replaces 3-OH of a bile acid or a bile acid analog or derivative, e.g., those described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, etc.

### R¹

In some embodiments, R¹ is R^{s}. In some embodiments, R¹ is —H. In some embodiments, R¹ is halogen. In some embodiments, R¹ is —F. In some embodiments, R¹ is —Cl. In some embodiments, R¹ is —Br. In some embodiments, R¹ is —CN. In some embodiments, R¹ is —N₃.

In some embodiments, R¹ is —C(O)R′, —S(O)₂R′, —OS(O)₂R′, —OP(O)(R′)₂, —SR′, or —N(R′)₂, wherein each R′ is independently as described herein.

In some embodiments, R¹ is —OR. In some embodiments, R¹ is —OR, wherein R is not hydrogen. In some embodiments, R¹ is —OH. In some embodiments, R¹ is —OC(O)R. In some embodiments, R is —H. In some embodiments, R is optionally substituted C₁₋₆ aliphatic.

In some embodiments, R¹ is —S(O)₂OR wherein R is as described herein. In some embodiments, R¹ is —S(O)₂OH. In some embodiments, R¹ is in a salt form, e.g., a sodium salt form.

In some embodiments, R¹ is a protected hydroxyl group. In some embodiments, R¹ is —OTBS.

In some embodiments, R¹ replaces 3-OH of a bile acid or a bile acid analog or derivative, e.g., those described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, etc.

In some embodiments, each of R¹ and R^{1a} is independently —H, R′, halogen, —CN, —N₃, —OR′, —C(O)R′, —S(O)₂R′, —OS(O)₂R′, —OP(O)(R′)₂, —SR′, —N(R′)₂, a protected hydroxyl group, or R¹ and R^{1a} attached to the same atom are taken together to form ═O or ═NR^{x}, wherein R′ is as described herein. In some embodiments, R¹ is —H. In some embodiments, R^{1a} is —H.

In some embodiments, each of R¹ and R^{1a} is independently —H.

In some embodiments, one of R¹ and R^{1a} is —H and the other is —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, one of R¹ and R^{1a} is —H and the other is a protected hydroxy group. In some embodiments, R¹ is —H. In some embodiments, R^{1a} is —H.

In some embodiments, each of R¹ and R^{1a} is independently —H or —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, R¹ is —H. In some embodiments, R^{1a} is —H.

In some embodiments, wherein one of R¹ and R^{1a} is —H and the other is halogen. In some embodiments, wherein one of R¹ and R^{1a} is —H and the other is —F. In some embodiments, wherein one of R¹ and R^{1a} is —H and the other is —Cl. In some embodiments, wherein one of R¹ and R^{1a} is —H and the other is —Br. In some embodiments, wherein one of R¹ and R^{1a} is —H and the other is —I. In some embodiments, R¹ is —H. In some embodiments, R^{1a} is —H. In some embodiments, R¹ is —F. In some embodiments, R^{1a} is —F.

In some embodiments, each of R¹ and R^{1a} is independently —H or halogen. In some embodiments, each of R¹ and R^{1a} is independently —H or —F. In some embodiments, each of R¹ and R^{1a} is independently —H or —Cl. In some embodiments, each of R¹ and R^{1a} is independently —H or —Br. In some embodiments, each of R¹ and R^{1a} is independently —H or —I. In some embodiments, R¹ is —H. In some embodiments, R^{1a} is —H. In some embodiments, R¹ is —H and R^{1a} is —F. In some embodiments, R^{1a} is —H and R¹ is —F.

In some embodiments, each of R¹ and R^{1a} is halogen. In some embodiments, each of R¹ and R^{1a} is —F. In some embodiments, each of R¹ and R^{1a} is —Cl. In some embodiments, each of R¹ and R^{1a} is —Br. In some embodiments, each of R¹ and R^{1a} is —I.

In some embodiments, one of R¹ and R^{1a} is —H and the other of R¹ and R^{1a} is —OS(O)₂R′, wherein R′ is as described herein. In some embodiments, R′ is H. In some embodiments, R′ is methyl. In some embodiments, R¹ is —H. In some embodiments, R^{1a} is —H.

In some embodiments, each of R¹ and R^{1a} is independently —H or —OS(O)₂R′, wherein R′ is as described herein. In some embodiments, R′ is H. In some embodiments, R′ is methyl. In some embodiments, R¹ is —H. In some embodiments, R^{1a} is —H.

In some embodiments, neither of R¹ and R^{1a} is —H.

In some embodiments, each of R¹ and R^{1a} is independently selected from —H, halogen, —OR′, or —C(O)R′, wherein R′ is as described herein. In some embodiments, each of R¹ and R^{1a} is independently selected from —H or halogen. In some embodiments, each of R¹ and R^{1a} is independently selected from —H or —F. In some embodiments, each of R¹ and R^{1a} is independently selected from —H or —OR′, wherein R′ is as described herein. In some embodiments, each of R¹ and R^{1a} is independently selected from —H or —OH. In some embodiments, each of R¹ and R^{1a} is —H. In some embodiments, each of R¹ and R^{1a} is independently selected from —H or —C(O)R′, wherein R′ is as described herein. In some embodiments, each of R¹ and R^{1a} is independently selected from —H or —C(O)OCH₃. In some embodiments, each of R¹ and R^{1a} is independently selected from —H or —S(O)₂R′, wherein R′ is as described herein. In some embodiments, each of each of R¹ and R^{1a} is independently selected from —H or —S(O)₂OR, wherein R is as described herein. In some embodiments, each of each of R¹ and R^{1a} is independently selected from —H or —S(O)₂OR, wherein R is as described herein. In some embodiments, each of each of R¹ and R^{1a} is independently selected from —H or —S(O)₂OH. In some embodiments, R¹ is —H. In some embodiments, R^{1a} is —H.

In some embodiments, R¹ and R^{1a} are taken together to form ═O. In some embodiments, R¹ and R^{1a} are taken together to form ═NR, wherein R is as described herein. In some embodiments, R^{x} is R′. In some embodiments, R^{x} is R as described herein. In some embodiments, R^{x} is R, wherein R is not —H. In some embodiments, R^{x} is optionally substituted C₁₋₆ aliphatic.

### R^{2a}

In some embodiments, R^{2a} is —H.

In some embodiments, R^{2a} is halogen. In some embodiments, R^{2a} is optionally substituted C₁₋₁₀ aliphatic. In some embodiments, R^{2a} is optionally substituted C₁₋₈ alkyl. In some embodiments, R^{2a} is C₁₋₈ alkyl. In some embodiments, R^{2a} is optionally substituted C₁₋₄ alkyl. In some embodiments, R^{2a} is C₁₋₄ alkyl. In some embodiments, R^{2a} is ethyl. In some embodiments, R^{2a} is optionally substituted C₂₋₈ alkynyl. In some embodiments, R^{2a} is optionally substituted C₃₋₈ cycloalkyl.

### R²

In some embodiments, R² is —H. In some embodiments, R² is halogen. In some embodiments, R² is optionally substituted C₁₋₁₀ aliphatic. In some embodiments, R² is optionally substituted C₁₋₈ alkyl. In some embodiments, R² is C₁₋₈ alkyl. In some embodiments, R² is optionally substituted C₁₋₄ alkyl. In some embodiments, R² is C₁₋₄ alkyl. In some embodiments, R² is ethyl. In some embodiments, R² is optionally substituted C₂₋₈ alkynyl. In some embodiments, R² is optionally substituted C₃₋₈ cycloalkyl.

In some embodiments, each of R² and R^{2a} is independently —H.

In some embodiments, each of R² and R^{2a} is independently halogen or —H. In some embodiments, one of R² and R^{2a} is halogen and the other is —H.

In some embodiments, each of R² and R^{2a} is independently optionally substituted C₁₋₁₀ aliphatic or —H. In some embodiments, each of R² and R^{2a} is independently optionally substituted C₁₋₈ alkyl or —H. In some embodiments, each of R² and R^{2a} is independently C₁₋₈ alkyl or —H. In some embodiments, each of R² and R^{2a} is independently optionally substituted C₁₋₄ alkyl or —H. In some embodiments, each of R² and R^{2a} is independently C₁₋₄ alkyl or —H. In some embodiments, each R² and R^{2a} is independently ethyl or —H. In some embodiments, each of R² and R^{2a} is independently optionally substituted C₂₋₈ alkynyl or —H. In some embodiments, each of R² and R^{2a} is independently optionally substituted C₃₋₈ cycloalkyl or —H. In some embodiments, one of R² and R^{2a} is optionally substituted C₁₋₁₀ aliphatic and the other is —H. In some embodiments, one of R² and R^{2a} is optionally substituted C₁₋₈ alkyl and the other is —H. In some embodiments, one of R² and R^{2a} is C₁₋₈ alkyl and the other is —H. In some embodiments, one of R² and R^{2a} is optionally substituted C₁₋₄ alkyl and the other is —H. In some embodiments, one of R² and R^{2a} is C₁₋₄ alkyl and the other is —H. In some embodiments, one of R² and R^{2a} is ethyl and the other is —H. In some embodiments, one of R² and R^{2a} is optionally substituted C₂₋₈ alkynyl and the other is —H. In some embodiments, one of R² and R^{2a} is optionally substituted C₃₋₈ cycloalkyl and the other is —H. In some embodiments, R^{2a} is —H. In some embodiments, R² is —H. In some embodiments, R^{2a} is —H and R² is optionally substituted C₁₋₆ aliphatic. In some embodiments, R^{2a} is —H and R² is ethyl.

In some embodiments, each of R² and R^{2a} is independently —H.

In some embodiments, each of R² and R^{2a} is independently halogen or —H. In some embodiments, one of R² and R^{2a} is halogen and the other is —H.

In some embodiments, each of R² and R^{2a} is independently optionally substituted C₁₋₁₀ aliphatic or —H. In some embodiments, each of R² and R^{2a} is independently optionally substituted C₁₋₈ alkyl or —H. In some embodiments, each of R² and R^{2a} is independently C₁₋₈ alkyl or —H. In some embodiments, each of R² and R^{2a} is independently optionally substituted C₁₋₄ alkyl or —H. In some embodiments, each of R² and R^{2a} is independently C₁₋₄ alkyl or —H. In some embodiments, each R² and R^{2a} is independently ethyl or —H. In some embodiments, each of R² and R^{2a} is independently optionally substituted C₂₋₈ alkynyl or —H. In some embodiments, each of R² and R^{2a} is independently optionally substituted C₃₋₈ cycloalkyl or —H. In some embodiments, one of R² and R^{2a} is optionally substituted C₁₋₁₀ aliphatic and the other is —H. In some embodiments, one of R² and R^{2a} is optionally substituted C₁₋₈ alkyl and the other is —H. In some embodiments, one of R² and R^{2a} is C₁₋₈ alkyl and the other is —H. In some embodiments, one of R² and R^{2a} is optionally substituted C₁₋₄ alkyl and the other is —H. In some embodiments, one of R² and R^{2a} is C₁₋₄ alkyl and the other is —H. In some embodiments, one of R² and R^{2a} is ethyl and the other is —H. In some embodiments, one of R² and R^{2a} is optionally substituted C₂₋₈ alkynyl and the other is —H. In some embodiments, one of R² and R^{2a} is optionally substituted C₃₋₈ cycloalkyl and the other is —H.

In some embodiments, R² is —H. In some embodiments, R² is halogen. In some embodiments, R² is optionally substituted C₁₋₁₀ aliphatic. In some embodiments, R² is optionally substituted C₁₋₈ alkyl. In some embodiments, R² is C₁₋₈ alkyl. In some embodiments, R² is optionally substituted C₁₋₄ alkyl. In some embodiments, R² is C₁₋₄ alkyl. In some embodiments, R² is ethyl. In some embodiments, R² is optionally substituted C₂₋₈ alkynyl. In some embodiments, R² is optionally substituted C₃₋₈ cycloalkyl.

### R^{3a}

In some embodiments, R^{3a} is —H. In some embodiments, R^{3a} is —OH.

In some embodiments, R^{3a} is halogen. In some embodiments, R^{3a} is R′ as described herein. In some embodiments, R^{3a} is —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

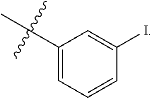 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl.

In some embodiments, R^{3a} is a protected hydroxyl group.

### R³

In some embodiments, R³ is —H. In some embodiments, R³ is —OH.

In some embodiments, R³ is halogen. In some embodiments, R³ is R′ as described herein. In some embodiments, R³ is —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

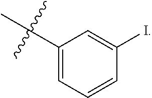 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl.

In some embodiments, R³ is a protected hydroxyl group.

In some embodiments, each of R³ and R^{3a} is independently —H.

In some embodiments, each of R³ and R^{3a} is independently halogen or —H. In some embodiments, one of R³ and R^{3a} is halogen and the other is —H. In some embodiments, R³ is —H. In some embodiments, R^{3a} is —H.

In some embodiments, one of R³ and R^{3a} is —H and the other is —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

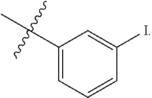 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, R³ is —H. In some embodiments, R^{3a} is —H.

In some embodiments, each of R³ and R^{3a} is independently —H, R′, or —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

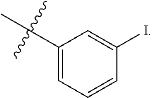 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, R³ is —H. In some embodiments, R^{3a} is —H.

In some embodiments, one of R³ and R^{3a} is —H and the other is —OH. In some embodiments, R³ is —H and R^{3a} is —OH. In some embodiments, R³ is —OH and R^{3a} is —H.

In some embodiments, one of R³ and R^{3a} is —H and the other is —R′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

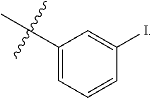 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, one of R³ and R^{3a} is —H and the other is —OR′, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —C(O)R, wherein R is

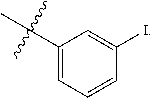 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl. In some embodiments, R³ is —H. In some embodiments, R^{3a} is —H.

In some embodiments, R³ and R^{3a} are taken together to form ═O.

In some embodiments, R³ and R^{3a} are taken together to form ═NR^{x}. In some embodiments, R^{x} is R′ as described herein. In some embodiments, R′ is —C(O)R wherein R is as described herein. In some embodiments, R^{x} is —OR wherein R is optionally substituted C₁₋₆ aliphatic. In some embodiments, R^{x} is —OR wherein R is optionally substituted C₁₋₆ alkyl. In some embodiments, R^{x} is —OR wherein R is methyl. In some embodiments, R^{x} is -L″-R′. In some embodiments, R^{x} is —O—C(R′)₂—C(O)OR′. In some embodiments, R^{x} is —O—C(CH₃)₂—C(O)OH.

### R^{4a}

In some embodiments, R^{4a} is R′ as described herein. In some embodiments, R^{4a} is R as described herein. In some embodiments, R^{4a} is —H.

In some embodiments, R^{4a} is —OR′ wherein R′ is as described herein. In some embodiments, R^{4a} is —OH. In some embodiments, R^{4a} is —OC(O)R wherein R is as described herein. In some embodiments, R^{4a} is —OC(O)H.

In some embodiments, R^{4a} is protected hydroxyl. In some embodiments, R^{4a} is —OTBS.

In some embodiments, R^{4a} is halogen. In some embodiments, R^{4a} is R′ as described herein. In some embodiments, R^{4a} is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R⁴

In some embodiments, R⁴ is R′ as described herein. In some embodiments, R⁴ is R as described herein. In some embodiments, R⁴ is —H.

In some embodiments, R⁴ is —OR′ wherein R′ is as described herein. In some embodiments, R⁴ is —OH. In some embodiments, R⁴ is —OC(O)R wherein R is as described herein. In some embodiments, R⁴ is —OC(O)H.

In some embodiments, R⁴ is protected hydroxyl. In some embodiments, R⁴ is —OTBS.

In some embodiments, R⁴ is halogen. In some embodiments, R⁴ is R′ as described herein. In some embodiments, R⁴ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, each of R⁴ and R^{4a} is independently —H.

In some embodiments, each of R⁴ and R^{4a} is independently halogen or —H. In some embodiments, one of R⁴ and R^{4a} is halogen and the other is —H. In some embodiments, R⁴ is —H. In some embodiments, R^{4a} is —H.

In some embodiments, each of R⁴ and R^{4a} is independently —H, R′, or —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, one of R⁴ and R^{4a} is —H and the other is —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R⁴ is —H and R^{4a} is —OR′ as described herein. In some embodiments, R⁴ is —H and R^{4a} is —OR′ as described herein. In some embodiments, R⁴ is —H and R^{4a} is —OC(O)R as described herein. In some embodiments, R^{4a} is —H and R⁴ is —OR′ as described herein. In some embodiments, R^{4a} is —H and R⁴ is —OR′ as described herein. In some embodiments, R^{4a} is —H and R⁴ is —OC(O)R as described herein.

In some embodiments, one of R⁴ and R^{4a} is —H and the other is —R′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R⁴ is —H. In some embodiments, R^{4a} is —H.

In some embodiments, R⁴ and R^{4a} are taken together to form ═O In some embodiments, R⁴ and R^{4a} are taken together to form ═NR wherein R is as described herein.

### R⁵

In some embodiments, R^{s} is —H.

In some embodiments, R^{s} is halogen. In some embodiments, R is —F. In some embodiments, R is —Cl. In some embodiments, R^{s} is —Br. In some embodiments, R is —I. In some embodiments, R^{s} is a leaving group. In some embodiments, R can be eliminated with R⁶ or R^{6a} which is hydrogen to form a double bond. Suitable leaving groups are available to those skilled in the art and can be utilized herein in accordance with the present disclosure.

In some embodiments, R is R′ as described herein. In some embodiments, R^{s} is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl.

### R^{6a}

In some embodiments, R^{6a} is —H. In some embodiments, R^{6a} is halogen.

In some embodiments, R^{6a} is R′ as described herein. In some embodiments, R^{6a} is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R⁶

In some embodiments, R⁶ is —H. In some embodiments, R⁶ is halogen.

In some embodiments, R⁶ is R′ as described herein. In some embodiments, R⁶ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, each of R⁶ and R^{6a} is independently —H.

In some embodiments, each of R⁶ and R^{6a} is independently halogen or —H. In some embodiments, one of R⁶ and R^{6a} is halogen and the other is —H.

In some embodiments, each of R⁶ and R^{6a} is independently —H, R′, or —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, one of R⁶ and R^{6a} is —H and the other is —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, one of R⁶ and R^{6a} is —H and the other is —R′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, R⁶ and R^{6a} are taken together to form ═O. In some embodiments, R⁶ and R^{6a} are taken together to form ═NR^{x} as described herein.

In some embodiments, R⁵ and R⁶ are taken together to form a covalent bond. In some embodiments, R^{s} and R^{6a} are taken together to form a covalent bond. Thus, in some embodiments, the bond between the carbons to which R⁵ and R⁶/R^{6a} are attached is a double bond.

### R^{7a}

In some embodiments, R^{7a} is —H. In some embodiments, R^{7a} is halogen.

In some embodiments, R^{7a} is R′ as described herein. In some embodiments, R^{7a} is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, R^{7a} is —OH. In some embodiments, R^{7a} is a protected hydroxyl group.

### R⁷

In some embodiments, R⁷ is —H. In some embodiments, R⁷ is halogen.

In some embodiments, R⁷ is R′ as described herein. In some embodiments, R⁷ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, R^{7a} is —OH. In some embodiments, R^{7a} is a protected hydroxyl group.

In some embodiments, each of R⁷ and R^{7a} is independently —H.

In some embodiments, each of R⁷ and R^{7a} is independently halogen or —H. In some embodiments, one of R⁷ and R^{7a} is halogen and the other is —H. In some embodiments, R⁷ is —H. In some embodiments, R^{7a} is —H.

In some embodiments, each of R⁷ and R^{7a} is independently —H, R′, or —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R⁷ is —H. In some embodiments, R^{7a} is —H.

In some embodiments, one of R⁷ and R^{7a} is —OH. In some embodiments, one of R⁷ and R^{7a} is —OH and the other is —H. In some embodiments, R⁷ is —H and R^{7a} is —OH. In some embodiments, R^{7a} is —H and R⁷ is —OH.

In some embodiments, one of R⁷ and R^{7a} is —H and the other is —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R⁷ is —H. In some embodiments, R^{7a} is —H.

In some embodiments, one of R⁷ and R^{7a} is —H and the other is —R′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl. In some embodiments, R⁷ is —H. In some embodiments, R^{7a} is —H.

In some embodiments, R⁷ and R^{7a} are taken together to form ═O. In some embodiments, R² and R^{7a} are taken together to form ═NR as described herein.

### R⁸

In some embodiments, R⁸ is —H. In some embodiments, R⁸ is halogen.

In some embodiments, R⁸ is R′ as described herein. In some embodiments, R⁸ is R as described herein. In some embodiments, R⁸ is optionally substituted C₁₋₆ aliphatic. In some embodiments, R⁸ is optionally substituted C₁₋₆ aliphatic alkyl.

In some embodiments, R⁸ is —OR′, wherein R′ is as described herein. In some embodiments, R′ is —H. In some embodiments, R′ is R as described herein. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R⁹

In some embodiments, R⁹ is —H. In some embodiments, R⁹ is halogen.

In some embodiments, R⁹ is R′ as described herein. In some embodiments, R⁹ is R as described herein. In some embodiments, R⁹ is —H. In some embodiments, R⁹ is optionally substituted C₁₋₆ aliphatic. In some embodiments, R⁹ is C₁₋₆ alkyl. In some embodiments, R⁹ is methyl.

In some embodiments, R⁹ is —OR′, wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R¹⁰

In some embodiments, R¹⁰ is —H. In some embodiments, R¹⁰ is halogen. In some embodiments, R¹⁰ is R′ as described herein. In some embodiments, R¹⁰ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R¹¹

In some embodiments, R¹¹ is —H. In some embodiments, R¹¹ is halogen. In some embodiments, R¹¹ is R′ as described herein. In some embodiments, R¹¹ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R¹²

In some embodiments, R¹² is —H. In some embodiments, R¹² is halogen. In some embodiments, R¹² is R′ as described herein. In some embodiments, R¹² is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R¹³

In some embodiments, R¹³ is —H. In some embodiments, R¹³ is halogen.

In some embodiments, R¹³ is R′ as described herein. In some embodiments, R¹³ is R as described herein. In some embodiments, R¹³ is —H. In some embodiments, R¹³ is C₁₋₆ aliphatic. In some embodiments, R¹³ is C₁₋₆ alkyl. In some embodiments, R¹³ is methyl.

In some embodiments, R¹³ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R^{14a}

In some embodiments, R^{14a} is —H. In some embodiments, R^{14a} is halogen.

In some embodiments, R^{14a} is R′ as described herein. In some embodiments, R^{14a} is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

### R¹⁴

In some embodiments, R¹⁴ is —H. In some embodiments, R¹⁴ is halogen.

In some embodiments, R¹⁴ is R′ as described herein. In some embodiments, R¹⁴ is —OR′ wherein R′ is as described herein. In some embodiments, R′ is —H. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆-C₁₂ aryl. In some embodiments, R′ is —C(O)R, wherein R is optionally substituted phenyl. In some embodiments, R′ is —CO₂R, wherein R is as described herein. In some embodiments, R′ is —CO₂R, wherein R is —H. In some embodiments, R′ is —CO₂R, wherein R is methyl.

In some embodiments, at least one R¹⁴ and R^{14a} is —H. In some embodiments, both R¹⁴ and R^{14a} are —H.

In some embodiments, one of R⁴ and R^{4a} and one of R¹⁴ and R^{14a} are taken together to form a covalent bond. Thus, in some embodiments, the bond between the carbons to which R⁴/R^{4a} and R¹⁴/R^{14a} are attached is a double bond.

### R¹⁵

In some embodiments, R¹⁵ is R^{s} as described herein. In some embodiments, R¹⁵ is R′ as described herein. In some embodiments, R¹⁵ is R as described herein. In some embodiments, R¹⁵ is —H as described herein.

### R¹⁶

In some embodiments, R¹⁶ is R^{s} as described herein. In some embodiments, R¹⁶ is R′ as described herein. In some embodiments, R¹⁶ is R as described herein. In some embodiments, R¹⁶ is —H as described herein.

### R¹⁷

In some embodiments, R¹⁷ is R^{s} as described herein. In some embodiments, R¹⁷ is R′ as described herein. In some embodiments, R¹⁷ is R as described herein. In some embodiments, R¹⁷ is —H as described herein.

### R^{18a}

In some embodiments, R^{18a} is R^{L} as described herein. In some embodiments, R^{18a} is —C(O)NHS(O)₂R^{L} wherein R^{L} is as described herein. In some embodiments, R^{18a} is —C(S)NHS(O)₂R^{L} wherein R^{L} is as described herein. In some embodiments, R^{18a} is —C(O)C(O)NHS(O)₂R^{L} wherein R^{L} is as described herein. In some embodiments, R^{18a} is —S(O)₂R^{L}, —C(O)NHR^{L} wherein R^{L} is as described herein.

In some embodiments, R^{18a} is R^{s} as described herein. In some embodiments, R^{18a} is R′ as described herein. In some embodiments, R^{18a} is R as described herein. In some embodiments, R^{18a} is —H as described herein.

### R¹⁸

In some embodiments, R¹⁸ is R^{s} as described herein. In some embodiments, R¹⁸ is R′ as described herein. In some embodiments, R¹⁸ is R as described herein. In some embodiments, R¹⁸ is —H as described herein.

### R^{19a}

In some embodiments, R^{19a} is R′ as described herein. In some embodiments, R^{19a} is R as described herein. In some embodiments, R^{19a} is —H as described herein.

### R¹⁹

In some embodiments, R¹⁹ is R′ as described herein. In some embodiments, R¹⁹ is R as described herein. In some embodiments, R¹⁹ is —H as described herein.

### R^{20a}

In some embodiments, R^{20a} is R as described herein. In some embodiments, R^{20a} is R′ as described herein. In some embodiments, R^{20a} is R as described herein. In some embodiments, R^{20a} is —H as described herein. In some embodiments, R^{20a} is taken together with a neighboring group which can be R (e.g., R¹, R^{1a}, etc.) to form a double bond. In some embodiments, R^{20a} is taken together with a neighboring group which can be R (e.g., R¹, R^{1a}, etc.) to form an optionally substituted ring as described herein.

### R²⁰

In some embodiments, R²⁰ is R^{s} as described herein. In some embodiments, R²⁰ is R′ as described herein. In some embodiments, R²⁰ is R as described herein. In some embodiments, R²⁰ is —H as described herein. In some embodiments, R²⁰ is taken together with a neighboring group which can be R (e.g., R¹, R^{1a}, etc.) to form a double bond. In some embodiments, R²⁰ is taken together with a neighboring group which can be R (e.g., R¹, R^{1a}, etc.) to form an optionally substituted ring as described herein.

### R^{21a}

In some embodiments, R^{21a} is R as described herein. In some embodiments, R^{21a} is R′ as described herein. In some embodiments, R^{21a} is R as described herein. In some embodiments, R^{21a} is —H as described herein. In some embodiments, R^{21a} is taken together with a neighboring group which can be R (e.g., R¹, R^{1a}, etc.) to form a double bond. In some embodiments, R^{21a} is taken together with a neighboring group which can be R (e.g., R¹, R^{1a}, etc.) to form an optionally substituted ring as described herein.

### R²¹

In some embodiments, R²¹ is R as described herein. In some embodiments, R²¹ is R′ as described herein. In some embodiments, R²¹ is R as described herein. In some embodiments, R²¹ is —H as described herein. In some embodiments, R²¹ is taken together with a neighboring group which can be R (e.g., R¹, R^{1a}, etc.) to form a double bond. In some embodiments, R²¹ is taken together with a neighboring group which can be R (e.g., R¹, R^{1a}, etc.) to form an optionally substituted ring as described herein. In some embodiments, R²¹ and R¹ are taken together with their intervening atoms to form an optionally substituted 5-10 membered ring as described herein. In some embodiments, a formed ring is an optionally substituted 5-6 membered aromatic ring having 0-4 (e.g., 0, 1, 2, 3, or 4) heteroatoms (as those skilled in the art will appreciate, in such embodiments, R^{1a} and R^{21a} will be absent). In some embodiments, a formed ring is an optionally substituted 5-6 membered aromatic ring having 1-4 (e.g., 1, 2, 3, or 4) heteroatoms independently selected from nitrogen, oxygen and sulfur.

### R^{22a}

In some embodiments, R^{22a} is R^{s} as described herein. In some embodiments, R^{22a} is R′ as described herein. In some embodiments, R^{22a} is R as described herein. In some embodiments, R^{22a} is —H as described herein. In some embodiments, R^{22a} is taken together with a neighboring group which can be R (e.g., R²¹, R^{22a}, etc.) to form a double bond. In some embodiments, R²⁰ is taken together with a neighboring group which can be R (e.g., R²¹, R^{21a}, etc.) to form an optionally substituted ring as described herein.

### R²²

In some embodiments, R²² is R^{s} as described herein. In some embodiments, R²² is R′ as described herein. In some embodiments, R²² is R as described herein. In some embodiments, R²² is —H as described herein. In some embodiments, R²² is taken together with a neighboring group which can be R (e.g., R²¹, R^{21a}, etc.) to form a double bond. In some embodiments, R²² is taken together with a neighboring group which can be R (e.g., R²¹, R^{21a}, etc.) to form an optionally substituted ring as described herein.

### R^{s}

In some embodiments, R^{s} is —H, -L″-R′, halogen, —CN, —N₃, —OR′, —C(O)R′, —S(O)₂R′, —S(O)₂N(R′)₂, —SO₃R′—OS(O)₂R′, —OP(O)(R′)₂, —OPO(OR′)₂, —P(O)(R′)₂, —PO(OR′)₂, —SR′, —C(O)N(R′)₂, —N(R′)₂, a protected hydroxyl group, or R^{s} is

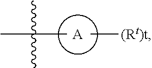

or two R^{s} attached to the same atom are taken together to form ═O or ═NR^{x}. In some embodiments, each R^{s} is independently —H, R′, halogen, —CN, —N₃, —OR′, —C(O)R′, —S(O)₂R′, —OS(O)₂R′, —OP(O)(R′)₂, —SR′, —N(R′)₂, a protected hydroxyl group, or two R^{s} attached to the same atom are taken together to form ═O or ═NR, wherein R′ is as described herein.

In some embodiments, R^{s} is —H. In some embodiments, each R^{s} is independently —H.

In some embodiments, R^{s} is -L″-R′ wherein each variable is as described herein. In some embodiments, R^{s} is -L″-R′ as described herein, e.g., in the section of certain embodiments for R^{L}. In some embodiments, L″ is a covalent bond. In some embodiments, L″ is an optionally substituted, bivalent C₁₋₆ aliphatic or heteroaliphatic group having 1-10 heteroatoms wherein one or more methylene units of the group are optionally and independently replaced as described herein. In some embodiments, L″ is an optionally substituted, bivalent C₁₋₄ aliphatic or heteroaliphatic group having 1-10 heteroatoms wherein one or more methylene units of the group are optionally and independently replaced as described herein. In some embodiments, L″ is optionally substituted, bivalent C₁₋₆ aliphatic wherein one or more methylene units of the group are optionally and independently replaced as described herein. In some embodiments, L″ is optionally substituted, bivalent C₁₋₄ aliphatic wherein one or more methylene units of the group are optionally and independently replaced as described herein. In some embodiments, at least one methylene unit is replaced as described herein. In some embodiments, at least one methylene unit is replaced with -Cy-, —O—, —S—, —S—S—, —N(R′)—, —C(O)—, —C(O)S—, —C(O)O—, —C(S)—, —C(NR′)—, —C(O)N(R′)—, —C(S)N(R′)—, —C(NR′)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, at least one methylene unit is replaced with -Cy-, —O—, —S—, —S—S—, —N(R′)—, —C(O)—, —C(O)S—, —C(O)O—, —C(S)—, —C(NR′)—, —C(O)N(R′)—, —C(S)N(R′)—, —C(NR′)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, or —S(O)₂N(R′)—. In some embodiments, R^{s} is R as described herein.

In some embodiments, R^{s} is halogen. In some embodiments, R^{s} is —F. In some embodiments, R^{s} is —Cl. In some embodiments, R^{s} is —Br. In some embodiments, R^{s} is —I. In some embodiments, R^{s} is —CN. In some embodiments, R^{s} is —N₃.

In some embodiments, R^{s} is —OR′ wherein R′ is as described herein. In some embodiments, R′ is —H. In some embodiments, R^{s} is —OR wherein R is as described herein. In some embodiments, R^{s} is —C(O)R′ wherein R′ is as described herein. In some embodiments, R^{s} is —S(O)₂R′ wherein R′ is as described herein. In some embodiments, R^{s} is —S(O)₂N(R′)₂ wherein each R′ is independently as described herein. In some embodiments, R^{s} is —SO₃R′ wherein R′ is as described herein. In some embodiments, R^{s} is —OS(O)₂R′ wherein R′ is as described herein. In some embodiments, R^{s} is —OP(O)(R′)₂ wherein each R′ is independently as described herein. In some embodiments, R^{s} is —OP(O)(OR′)₂ wherein each R′ is independently as described herein. In some embodiments, R^{s} is —P(O)(R′)₂ wherein each R′ is independently as described herein. In some embodiments, R^{s} is —PO(OR′)₂ wherein each R′ is independently as described herein. In some embodiments, R^{s} is —SR′ wherein each R′ is as described herein. In some embodiments, R^{s} is —C(O)N(R′)₂ wherein each R′ is independently as described herein. In some embodiments, R^{s} is —N(R′)₂ wherein each R′ is independently as described herein.

In some embodiments, R^{s} is optionally substituted C₁₋₁₀ aliphatic. In some embodiments, R^{s} is optionally substituted C₁₋₆ aliphatic. In some embodiments, R^{s} is optionally substituted C₁₋₆ alkyl. In some embodiments, R^{s} is methyl. In some embodiments, R^{s} is ethyl. In some embodiments, R^{s} is t-butyl.

In some embodiments, each R^{s} is independently —H or optionally substituted C₁₋₁₀ aliphatic. In some embodiments, each R^{s} is independently —H or optionally substituted C₁₋₆ aliphatic. In some embodiments, each R^{s} is independently —H or optionally substituted C₁₋₆ alkyl. In some embodiments, each R^{s} is independently —H or methyl. In some embodiments, each R^{s} is independently —H or ethyl.

In some embodiments, each R^{s} is independently —H, —OR′, or optionally substituted C₁₋₁₀ aliphatic, wherein R′ is as described herein. In some embodiments, each R^{s} is independently —H, —OR′, or optionally substituted C₁₋₆ aliphatic, wherein R′ is as described herein. In some embodiments, each R^{s} is independently —H, —OR′, or optionally substituted C₁₋₆ alkyl, wherein R′ is as described herein. In some embodiments, each R^{s} is independently —H, —OR′, or methyl, wherein R′ is as described herein. In some embodiments, each R^{s} is independently —H, —OR′, or ethyl, wherein R′ is as described herein. In some embodiments, R′ is —H. In some embodiments, R′ is —S(O)₂R, wherein R is as described herein. In some embodiments, R′ is —S(O)₂R, wherein R is —H. In some embodiments, R′ is —S(O)₂R, wherein R is methyl. In some embodiments, R′ is —C(O)R, wherein R is as described herein. In some embodiments, R′ is —C(O)R, wherein R is —H. In some embodiments, R′ is —C(O)R, wherein R is methyl. In some embodiments, R′ is —C(O)R, wherein R is optionally substituted C₆₋₁₄ aryl. In some embodiments, R′ is —C(O)R, wherein R is optionally substituted phenyl. In some embodiments, R′ is —C(O)R, wherein R is

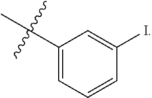 [SMILES: *c1cccc(I)c1]

In some embodiments, R′ is —CO₂R, wherein R is as described herein. In some embodiments, R′ is —CO₂R, wherein R is —H. In some embodiments, R′ is —CO₂R, wherein R is methyl. In some embodiments, R′ is C₁₋₁₀ aliphatic. In some embodiments, R′ is C₁₋₆ aliphatic. In some embodiments, R′ is C₁₋₆ alkyl. In some embodiments, R′ is methyl.

In some embodiments, R^{s} is a protected hydroxyl group. Various technologies for protecting a hydroxyl group are available and can be utilized in accordance with the present disclosure.

In some embodiments, R^{s} is

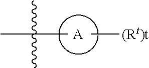

wherein each variable is independently as described herein.

In some embodiments, two R^{s} attached to the same atom are taken together to form ═O.

In some embodiments, two R^{s} attached to the same atom are taken together to form ═NR^{x} wherein R^{x} is as described herein. In some embodiments, R^{x} is —OR′, wherein R′ is optionally substituted C₁₋₁₀ aliphatic. In some embodiments, R^{x} is —OR′ wherein R′ is optionally substituted C₁₋₆ aliphatic. In some embodiments, R′ is optionally substituted C₁₋₆ alkyl. In some embodiments, R^{x} is

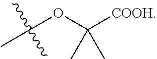 [SMILES: *OC(C)(C)C(=O)O]

In some embodiments, R^{s} is R₁ as described herein. In some embodiments, R^{s} is R₁ as described in, e.g., Table 1 to Table 7.

### L¹

In some embodiments, L¹ is L, and wherein L is a covalent bond.

In some embodiments, L¹ is L, and wherein L is an optionally substituted bivalent C₁-C₁₂ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L¹ is L, and wherein L is an optionally substituted bivalent C₁-C₁₂ aliphatic group. In some embodiments, L is a bivalent C₁-C₁₂ aliphatic group substituted with one or more C₁-C₁₀ aliphatic groups. In some embodiments, L is a bivalent C₁-C₁₂ aliphatic group substituted with methyl. In some embodiments, L is a bivalent C₁-C₁₂ aliphatic group substituted with ethyl. In some embodiments, L is a bivalent C₁-C₁₂ aliphatic group substituted with propyl. In some embodiments, L is a bivalent C₁-C₁₂ aliphatic group substituted with isopropyl. In some embodiments, L is a bivalent C₁-C₁₂ aliphatic group substituted with n-butyl. In some embodiments, L is a bivalent C₁-C₁₂ aliphatic group substituted with iso-butyl. In some embodiments, L is a bivalent C₁-C₁₂ aliphatic group substituted with —OR wherein R is as described herein. In some embodiments, L is a bivalent C₁-C₁₂ aliphatic group substituted with —OH. In some embodiments, L is a bivalent C₁-C₁₂ aliphatic group substituted with —CN. In some embodiments, L is a bivalent C₁-C₁₂ aliphatic group substituted with —NO₂. In some embodiments, L is a bivalent C₁-C₁₂ aliphatic group substituted with —NR₂ wherein each R is independently as described herein.

In some embodiments, L¹ is L, and wherein L is an optionally substituted bivalent C₁-C₁₂ alkylene group. In some embodiments, L¹ is L, and wherein L is an optionally substituted bivalent C₁-C₆ alkenylene group. In some embodiments, L¹ is L, and wherein L is an optionally substituted bivalent C₁-C₃ alkenylene group. In some embodiments, L¹ is L, and wherein L is an optionally substituted bivalent C₁-C₃ alkenylene group.

In some embodiments, L¹ is L, and wherein L is an optionally substituted

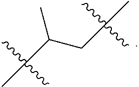 [SMILES: *CC(*)C]

In some embodiments, L¹ is L, and wherein L is an optionally substituted

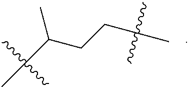 [SMILES: *CCC(*)C]

In some embodiments, L¹ is L, and wherein L is an optionally substituted

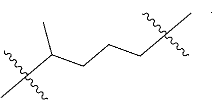 [SMILES: *CCCC(*)C]

In some embodiments, L¹ is L, and wherein L is an optionally substituted

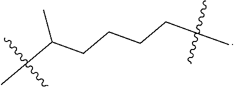 [SMILES: *CCCCC(*)C]

In some embodiments, L¹ is L, and wherein L is an optionally substituted

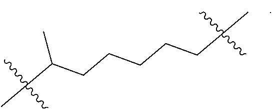 [SMILES: *CCCCCC(*)C]

In some embodiments, L¹ is L, and wherein L is an optionally substituted

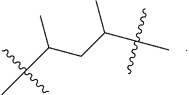 [SMILES: *C(C)CC(*)C]

In some embodiments, L¹ is L, and wherein L is an optionally substituted

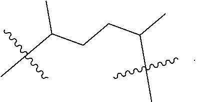 [SMILES: *C(C)CCC(*)C]

In some embodiments, L¹ is L, and wherein L is an optionally substituted

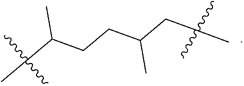 [SMILES: *CC(C)CCC(*)C]

As appreciated by those skilled in the art, a bivalent group as described therein can be connected to the rest of the molecule in either direction. For example, the present disclosure contemplates a bivalent group

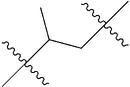 [SMILES: *CC(*)C]

to be connected to the rest of the molecule as either

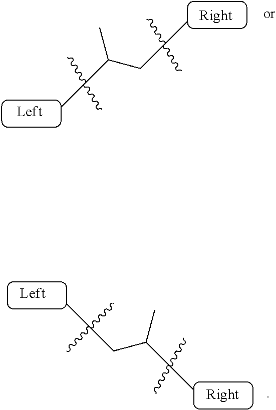

In some embodiments, L¹ is L, and wherein L is an optionally substituted

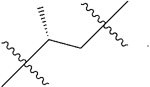 [SMILES: *C[C@H](*)C]

In some embodiments, L¹ is L, and wherein L is an optionally substituted

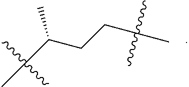 [SMILES: *CC[C@H](*)C]

In some embodiments, L¹ is L, and wherein L is an optionally substituted

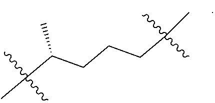 [SMILES: *CCC[C@H](*)C]

In some embodiments, L¹ is L, and wherein L is an optionally substituted

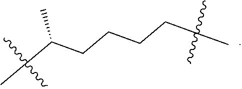 [SMILES: *CCCC[C@H](*)C]

In some embodiments, L¹ is L, and wherein L is an optionally substituted

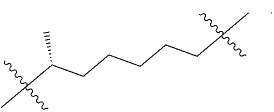 [SMILES: *CCCCC[C@H](*)C]

In some embodiments, L¹ is L, and wherein L is an optionally substituted

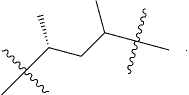 [SMILES: *C(C)C[C@H](*)C]

In some embodiments, L¹ is L, and wherein L is an optionally substituted

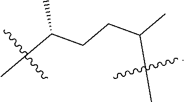 [SMILES: *C(C)CC[C@H](*)C]

In some embodiments, L¹ is L, and wherein L is an optionally substituted

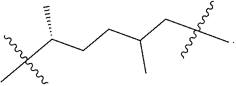 [SMILES: *CC(C)CC[C@H](*)C]

In some embodiments, L¹ is L, and wherein L is an optionally substituted bivalent C₂-C₁₂ alkenylene group, wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L¹ is L, and wherein L is an optionally substituted bivalent C₂-C₆ alkenylene group. In some embodiments, L¹ is L, and wherein L is an optionally substituted

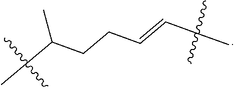 [SMILES: */C=C/CCC(*)C]

In some embodiments, L¹ is L, and wherein L is an optionally substituted

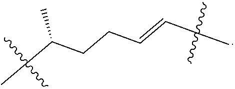 [SMILES: */C=C/CC[C@H](*)C]

In some embodiments, L¹ is L, and wherein L is an optionally substituted bivalent C₁-C₁₂ alkynylene group. In some embodiments, L¹ is L, and wherein L is an optionally substituted bivalent C₁-C₆ alkynylene group.

In some embodiments, L¹ is L, and wherein L is an optionally substituted bivalent C₁-C₁₂ aliphatic group wherein one or more methylene units of the group are optionally and independently replaced with —N(R′)—, wherein R′ is as described therein. In some embodiments, L¹ is L, and wherein L is an optionally substituted bivalent C₁-C₁₂ aliphatic group wherein one or more methylene units of the group are optionally and independently replaced with —N(H)—. In some embodiments, L¹ is L, and wherein L is an optionally substituted bivalent C₁-C₁₂ aliphatic group wherein one or more methylene units of the group are optionally and independently replaced with —N(Me)-.

In some embodiments, L¹ is L, and wherein L is an optionally substituted bivalent C₁-C₁₂ aliphatic group wherein one or more methylene units of the group are optionally and independently replaced with —C(O)—.

In some embodiments, L¹ is L, and wherein L is an optionally substituted bivalent C₁-C₁₂ aliphatic group wherein one or more methylene units of the group are optionally and independently replaced with —C(O)O—.

In some embodiments, L¹ is L, and wherein L is an optionally substituted bivalent C₁-C₁₂ aliphatic group wherein one or more methylene units of the group are optionally and independently replaced with —N(R′)C(O)N(R′)— wherein each R′ is independently as described herein. In some embodiments, L¹ is L, and wherein L is an optionally substituted bivalent C₁-C₁₂ aliphatic group wherein one or more methylene units of the group are optionally and independently replaced with —N(H)C(O)N(H)—.

In some embodiments, L¹ is L, and wherein L is an optionally substituted bivalent C₁-C₁₂ aliphatic group wherein one or more methylene units of the group are optionally and independently replaced with —S(O)₂—.

In some embodiments, L¹ is L, and wherein L is an optionally substituted bivalent C₁-C₁₂ aliphatic group wherein one or more methylene units of the group are optionally and independently replaced with —N(R′)— and —C(O)—.

In some embodiments, L¹ is L, and wherein L is an optionally substituted bivalent C₁-C₁₂ aliphatic group wherein one or more methylene units of the group are optionally and independently replaced with —N(R′)C(O)N(R′)— and —S(O)₂—.

In some embodiments, L¹ is —CH₂-L¹-, wherein —CH₂— is optionally substituted, L′ is bonded to R^{L}, and L′ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L′ is an optionally substituted C₁-C₁₁ aliphatic group. In some embodiments, L′ is an optionally substituted C₁-C₁₁ aliphatic group, wherein one or more methylene units of the group are optionally and independently replaced with —C(O)N(R′)—, —N(R′)C(O)N(R′)—, —S(O)₂—, or —S(O)₂N(R′)—.

In some embodiments, L¹ is —CHR′-L′-, wherein L′ is bonded to R^{L}, and L′ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH≡CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L′ is an optionally substituted C₁-C₁₁ aliphatic group. In some embodiments, L′ is an optionally substituted C₁-C₁₁ aliphatic group, wherein one or more methylene units of the group are optionally and independently replaced with —C(O)N(R′)—, —N(R′)C(O)N(R′)—, —S(O)₂—, or —S(O)₂N(R′)—. In some embodiments, R′ of —CHR′— is optionally substituted C₁₋₆ aliphatic. In some embodiments, R′ of —CHR′— is optionally substituted C₁₋₆ alkyl.

In some embodiments, L¹ is —CH(CH₃)-L′-, wherein —CH₂— is optionally substituted, L′ is bonded to R^{L}, and L′ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L′ is an optionally substituted C₁—C₁₁ aliphatic group. In some embodiments, L′ is an optionally substituted C₁-C₁₁ aliphatic group, wherein one or more methylene units of the group are optionally and independently replaced with —C(O)N(R′)—, —N(R′)C(O)N(R′)—, —S(O)₂—, or —S(O)₂N(R′)—.

In some embodiments, L¹ is -L⁰-L′-, wherein L^{c} is optionally substituted —CH₂—. In some embodiments, L¹ is —CHR′-L′-. In some embodiments, L¹ is —CH(CH₃)-L′-.

In some embodiments, L′ is -L^{s1}-L^{s2}-, wherein L^{s2} is bonded to R^{L}, L^{s1} is a covalent bond or an optionally substituted, bivalent C₁-C₆ aliphatic or heteroaliphatic group having 1-5 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, and L^{s2} is a covalent bond or an optionally substituted, bivalent C₁-C₅ aliphatic or heteroaliphatic group having 1-5 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, L^{s1} is optionally substituted —(CH₂)ₙ— wherein n is 1, 2, 3, 4, 5, or 6. In some embodiments, n is 1. In some embodiments, n is 2. In some embodiments, n is 3. In some embodiments, n is 4. In some embodiments, n is 5. In some embodiments, n is 6.

In some embodiments, L^{s1} is optionally substituted —(CH₂)n-C(R′)₂— wherein n is 1, 2, 3, 4, or 5. In some embodiments, L^{s1} is —(CH₂)n-C(R′)₂— wherein n is 1, 2, 3, 4, or 5. In some embodiments, L^{s1} is optionally substituted —(CH₂)n-CHR′— wherein n is 1, 2, 3, 4, or 5. In some embodiments, L^{s1} is —(CH₂)n-CHR′— wherein n is 1, 2, 3, 4, or 5. In some embodiments, each R′ is independently —H or optionally substituted C₁₋₆ aliphatic. In some embodiments, each R′ is independently optionally substituted C₁₋₆ aliphatic. In some embodiments, each R′ is independently optionally substituted C₁₋₆ alkyl. In some embodiments, each R′ is independently methyl. In some embodiments, L^{s1} is —(CH₂)n-CH(CH₃)—. In some embodiments, L^{s1} is —(CH₂)n-C(CH₃)₂—.

In some embodiments, L^{s1} is a covalent bond.

In some embodiments, L^{s2} is or comprises —O—. In some embodiments, L^{s2} is or comprises —S(O)₂—. In some embodiments, L^{s2} is or comprises —C(O)—. In some embodiments, —(O)—, —S(O)₂—, or —C(O)— is bonded to L^{s1}. In some embodiments, —(O)—, —S(O)₂—, or —C(O)— is bonded to R^{L}.

In some embodiments, L^{s2} is or comprises —C(O)—. In some embodiments, L^{s2} is or comprises —C(O)O—. In some embodiments, L^{s2} is or comprises —C(O)N(R′)—. In some embodiments, —C(O)N(R′)S(O)₂—. Various embodiments for R′ are as described herein. In some embodiments, —C(O)— is bonded to L^{s1}. In some embodiments, —C(O)— is bonded to R^{L}.

In some embodiments, L^{s2} is or comprises —N(R′)—. In some embodiments, L^{s2} is or comprises —N(R′)S(O)₂—. In some embodiments, L^{s2} is or comprises —N(R′)C(O)N(R′)S(O)₂—. In some embodiments, —N(R′)C(O)N(R′)—. Various embodiments for R′ are as described herein. In some embodiments, each R′ is independently —H or optionally substituted C₁₋₆ aliphatic. In some embodiments, each R′ is independently —H. In some embodiments, —N(R′)— is bonded to L^{s1}. In some embodiments, —N(R′)— is bonded to R^{L}.

In some embodiments, L^{s2} is or comprises -Cy-. In some embodiments, -Cy- is bonded to L^{s1}. In some embodiments, -Cy- is bonded to R^{L}. In some embodiments, L^{S2} is or comprises —N(R′)C(O)N(R′)S(O)₂-Cy-L″-, wherein L″ is a covalent bond, or an optionally substituted, bivalent C₁-C₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{s2} is or comprises —N(R′)C(O)N(R′)S(O)₂-Cy-L″- wherein each R′ is independently —H or optionally substituted C₁₋₆ aliphatic. In some embodiments, L^{s2} comprises —NHC(O)NHS(O)₂-Cy-L″-. Various useful embodiments for R′, -Cy-, L″, etc. are independently as described herein. In some embodiments, L″ is bonded to L^{s1}. In some embodiments, L″ is bonded to R^{L}.

In some embodiments, L^{s2} is or comprises optionally substituted —CH═CH—. In some embodiments, L^{s2} is or comprises —CH═CH—. In some embodiments, L^{s2} is or comprises optionally substituted —CH═CH—C(O)O—. In some embodiments, L^{s2} is or comprises —C(CH₃)=CH—C(O)O—. In some embodiments, L^{s2} is or comprises —CH═CH—C(O)O—. In some embodiments, the double bond is E. In some embodiments, the double bond is Z. In some embodiments, the double bond is bonded to L^{s}. In some embodiments, the double bond is bonded to R^{L}.

In some embodiments, L^{s2} is —C(O)O—, wherein —O— is bonded to R^{L}. In some embodiments, L^{s2} is —C(O)O—, wherein —O— is bonded to R^{s1}.

In some embodiments, L^{s2} is —C(O)N(R′)—, wherein R′ is —H or optionally substituted C₁₋₆ aliphatic. In some embodiments, L^{s2} is —C(O)NH—. In some embodiments, —C(O)— is bonded to L^{s1}. In some embodiments, —C(O)— is bonded to R^{L}.

In some embodiments, L^{s2} is —C(O)N(R′)S(O)₂—, wherein R′ is —H or optionally substituted C₁₋₆ aliphatic. In some embodiments, L^{s2} is —C(O)NHS(O)₂—. In some embodiments, —S(O)₂— is bonded to R^{L}.

In some embodiments, L^{s2} is —N(R′)C(O)N(R′)S(O)₂—, wherein R′ is —H or optionally substituted C₁₋₆ aliphatic. In some embodiments, L^{s2} is —N(H)C(O)N(H)S(O)₂—. In some embodiments, —S(O)₂— is bonded to R^{L}.

In some embodiments, L^{s2} is —N(R′)C(O)N(R′)S(O)₂-L″-. In some embodiments, L″ is a covalent bond. In some embodiments, L″ is optionally substituted C₁₋₁₀ aliphatic. In some embodiments, L″ is optionally substituted C₁₋₆ aliphatic. In some embodiments, L″ is optionally substituted C₁₋₆ alkyl. In some embodiments, L″ is optionally substituted —(CH₂)ₙ—, wherein n is 1, 2, 3, 4, 5, or 6. In some embodiments, n is 1. In some embodiments, n is 2. In some embodiments, n is 3. In some embodiments, n is 4. In some embodiments, n is 5. In some embodiments, n is 6.

In some embodiments, L^{s2} is —N(R′)C(O)N(R′)S(O)₂-Cy-L″-, wherein L″ is a covalent bond, or an optionally substituted, bivalent C₁-C₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, each R′ is independently —H or optionally substituted C₁₋₆ aliphatic. In some embodiments, L^{s2} is —NHC(O)NHS(O)₂-Cy-L″-.

In some embodiments, Cy is an optionally substituted bivalent C₃₋₁₀ cycloaliphatic ring. In some embodiments, an optionally substituted bivalent C₃₋₁₀ cycloalkyl ring. In some embodiments, Cy is an optionally substituted bivalent cyclohexyl ring. In some embodiments, Cy is a bivalent cyclohexyl ring.

In some embodiments, Cy is an optionally substituted bivalent C₆₋₁₄ aryl. In some embodiments, Cy is optionally substituted phenyl. In some embodiments, Cy is phenyl. In some embodiments, Cy is an optionally substituted monocyclic or bicyclic 3-10 membered heterocyclyl ring having 1-5 heteroatoms, In some embodiments, Cy is an optionally substituted monocyclic 5- or 6-membered heterocyclyl ring having 1-3 heteroatoms. In some embodiments, Cy is an optionally substituted bicyclic 3-10 membered heterocyclyl ring having 1-3 heteroatoms. In some embodiments, Cy is an optionally substituted monocyclic 5- or 6-membered heterocyclyl ring having 1-3 nitrogen atoms. In some embodiments, Cy is an optionally substituted monocyclic 5- or 6-membered heterocyclyl ring having 1-3 nitrogen atoms and a nitrogen atom is bonded to —S(O)₂—. In some embodiments, Cy is

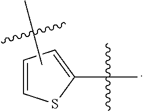

In some embodiments, Cy is

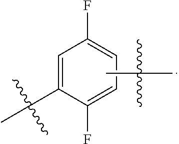

In some embodiments, Cy is

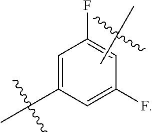

In some embodiments, L″ is bonded to R^{L}. In some embodiments, L″ is a covalent bond. In some embodiments, L″ is optionally substituted C₁₋₁₀ aliphatic. In some embodiments, L″ is optionally substituted C₁₋₆ aliphatic. In some embodiments, L″ is optionally substituted C₁₋₆ alkyl. In some embodiments, L″ is optionally substituted —(CH₂)ₙ—, wherein n is 1, 2, 3, 4, 5, or 6. In some embodiments, n is 1. In some embodiments, n is 2. In some embodiments, n is 3. In some embodiments, n is 4. In some embodiments, n is 5. In some embodiments, n is 6. In some embodiments, L″ is optionally substituted —(CH₂)—. In some embodiments, L″ is optionally substituted —(CH₂)₂—. In some embodiments, L″ is —C(CH₃)₂—. In some embodiments, L″ is —C(CH₃)(CH₂OH)—. In some embodiments, L″ is —C(R′)₂—(CH₂)n-, wherein each —CH₂— is optionally substituted and n is 0 or 1, and each R′ is as independently described herein. In some embodiments, two R′ of —C(R′)₂— are taken together with the carbon atom to which they are attached to form an optionally substituted 3-10 membered cycloaliphatic ring. In some embodiments, a formed ring is 3-membered. In some embodiments, a formed ring is 4-membered. In some embodiments, a formed ring is 3-membered. In some embodiments, a formed ring is 5-membered. In some embodiments, a formed ring is 6-membered.

In some embodiments, L″ is optionally substituted

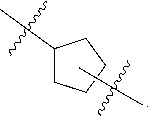

In some embodiments, L″ is optionally substituted

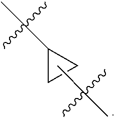

In some embodiments, L¹ is -L^{a}-L^{b}-L^{c}-, wherein:

L^{a} is a covalent bond, or an optionally substituted, bivalent C₁₋₂ aliphatic or heteroaliphatic group having 1-2 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;

L^{b} is a covalent bond, or an optionally substituted, bivalent C₁₋₁₀ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;

L^{c} is a covalent bond, or an optionally substituted, bivalent C₁₋₃ aliphatic or heteroaliphatic group having 1-3 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—; and

L^{c} is bonded to R^{L}.

### L^{a}

In some embodiments, L^{a} is a covalent bond. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—.

### L^{b}

In some embodiments, L^{b} is a covalent bond.

In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, L^{b} is optionally substituted —CH₂—. In some embodiments, L^{b} is —CH₂—. In some embodiments, L^{b} is optionally substituted —CH₂—CH₂—. In some embodiments, L^{b} is —CH₂—CH₂—.

In some embodiments, a methylene unit bonded to L^{c} is replaced with —C(R′)₂—. In some embodiments, a methylene unit bonded to L^{c} is replaced with —CHR′—. In some embodiments, R′ is R₃ as described herein. In some embodiments, L^{b} is —(CH₂)m-CH(R′)— as described herein. In some embodiments, L^{b} is —(CH₂)m-CH(R₃)— as described herein. In some embodiments, —(CH₂)m- is bonded to L^{a}. In some embodiments, L is —CH₂—O—. In some embodiments, L^{b} is —CH₂—OC(O)—. In some embodiments, L^{b} is —CH₂—OC(O)N(R′)—. In some embodiments, L^{b} is —CH₂—OC(O)NH—. In some embodiments, L^{b} is —CH₂—OC(O)N(R′)S(O)₂—. In some embodiments, L^{b} is —CH₂—OC(O)NHS(O)₂—.

### L^{c}

In some embodiments, L^{c} is a covalent bond.

In some embodiments, L^{c} is optionally substituted bivalent C₁₋₁₀ aliphatic. In some embodiments, L^{c} is C₁₋₁₀ is optionally substituted alkylene. In some embodiments, L^{c} is optionally substituted —(CH₂)₁₋₁₀—. In some embodiments, L^{c} is —(CH₂)m- as described herein. In some embodiments, L^{c} is —CH₂—. In some embodiments, L^{c} is —CH(COOH)—. In some embodiments, L^{c} is —CH(CN)—. In some embodiments, L^{c} is —C(R′)₂—. In some embodiments, L^{c} is —CHR′—. In some embodiments, L^{c} is —CH(CH₃)—.

In some embodiments, L^{c} is —O—. In some embodiments, L^{c} is —C(O)—. In some embodiments, L^{c} is —OC(O)—. In some embodiments, L^{c} is —OC(O)N(R′)—. In some embodiments, L^{c} is —OC(O)NH—. In some embodiments, L^{c} is —N(R′)C(O)N(R′)—. In some embodiments, L^{c} is —NHC(O)NH—. In some embodiments, L^{c} is —OC(O)N(R′)S(O)₂—. In some embodiments, L^{c} is —OC(O)NHS(O)₂—. In some embodiments, L^{c} is —S(O)₂—. In some embodiments, L^{c} is —N(R′)S(O)₂—. In some embodiments, L^{c} is —NHS(O)₂—. In some embodiments, L^{c} is —C(O)N(R′)S(O)₂—. In some embodiments, L^{c} is —C(O)NHS(O)₂—. In some embodiments, L^{c} is —OC(O)N(R′)S(O)₂—. In some embodiments, L^{c} is —OC(O)NHS(O)₂—. In some embodiments, L^{c} is —N(R′)—. In some embodiments, L^{c} is —NH—. In some embodiments, L^{c} is —N(R′)C(O)N(R′)—. In some embodiments, L^{c} is —NHC(O)NH—. In some embodiments, L^{c} is —N(R′)C(O)N(R′)S(O)₂—. In some embodiments, L^{c} is —NHC(O)NHS(O)₂—. In some embodiments, —S(O)₂— is bonded to R^{L}. In some embodiments, —S(O)₂— is bonded to R₁. In some embodiments, each R′ is independently —H or C₁₋₆ aliphatic. In some embodiments, each R′ is independently —H. In some embodiments, two R′ are taken together with their intervening atoms to form an optionally substituted ring as described herein.

In some embodiments, L^{c} is -Cy- as described herein. In some embodiments, -Cy- is an optionally substituted 3-10 membered ring having 0-4 heteroatoms independently selected from nitrogen, oxygen and sulfur. In some embodiments, -Cy- is an optionally substituted 5-membered heteroaryl ring having 1-4 heteroatoms independently selected from nitrogen, oxygen and sulfur. In some embodiments, -Cy-is

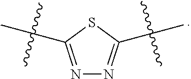 [SMILES: *c1nnc(*)s1]

In some embodiments, -Cy- is

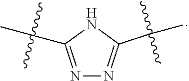 [SMILES: *c1nnc(*)[nH]1]

In some embodiments, L^{c} is —C(O)N(R′)S(O)₂— wherein R′ is as described herein. L^{c} is —C(O)N(R′)C(R′)₂C(O)N(R′)— wherein each R′ is independently as described herein. In some embodiments, L^{c} is —C(O)N(R′)C(R′)₂S(O)₂N(R′)— wherein each R′ is independently as described herein. In some embodiments, L^{c} is —C(O)N(R′)C(R′)₂P(O)(R′)— wherein each R′ is independently as described herein. In some embodiments, L^{c} is —C(O)N(R′)C(R′)₂N(R′)C(O)N(R′)S(O)₂— wherein each R′ is independently as described herein. In some embodiments, L^{c} is —C(O)N(R′)C(R′)₂— wherein each R′ is independently as described herein. In some embodiments, L^{c} is —C(O)N(R′)C(R′)₂C(O)N(R′)S(O)₂— wherein each R′ is independently as described herein. In some embodiments, L^{c} is —C(O)N(R′)S(O)₂N(R′)— wherein each R′ is independently as described herein. In some embodiments, L^{c} is —C(O)N(R′)C(NR′)N(R′)— wherein each R′ is independently as described herein. In some embodiments, L^{c} is —C(O)NHS(O)₂—. In some embodiments, L^{c} is —C(O)NHCH₂C(O)NH—. In some embodiments, L^{c} is —C(O)NHCH₂S(O)₂NH—. In some embodiments, L^{c} is —C(O)NHCH₂P(O)(R′)—. In some embodiments, L^{c} is —C(O)NHCH₂NHC(O)NHS(O)₂—. In some embodiments, L^{c} is —C(O)NHCH₂—. In some embodiments, L^{c} is —C(O)NHCH₂C(O)NHS(O)₂—. In some embodiments, L^{c} is —C(O)NHS(O)₂NH—. In some embodiments, L^{c} is —C(O)NHC(NR′)NH—.

In some embodiments, L^{c} is —P(O)(R′)— wherein R′ is as described herein. In some embodiments, L^{c} is —OS(O)₂O—. In some embodiments, L^{c} is —N(R′)C(O)— wherein R′ is as described herein. In some embodiments, L^{c} is —N(R′)C(S)— wherein R′ is as described herein. In some embodiments, L^{c} is —N(R′)C(NR′)— wherein each R′ is independently as described herein. In some embodiments, L^{c} is —N(R′)C(O)O— wherein R′ is as described herein. In some embodiments, L^{c} is —N(R′)C(NR′)N(R′)— wherein each R′ is independently as described herein. In some embodiments, L^{c} is —N(R′)C(S)N(R′)S(O)₂— wherein each R′ is independently as described herein. In some embodiments, L^{c} is —N(R′)C(NR′)N(R′)S(O)₂— wherein each R′ is independently as described herein. In some embodiments, L^{c} is —N(R′)C(O)C(O)N(R′)S(O)₂— wherein each R′ is independently as described herein. In some embodiments, L^{c} is —N(R′)C(O)N(R′)S(O)₂N(R′)— wherein each R′ is independently as described herein. In some embodiments, L^{c} is —N(R′)S(O)₂— wherein R′ is as described herein. In some embodiments, L^{c} is —OC(O)N(R′)— wherein R′ is as described herein. In some embodiments, L^{c} is —OC(O)N(R′)C(O)N(R′)— wherein each R′ is independently as described herein. In some embodiments, L^{c} is —OC(O)N(R′)C(O)— wherein R′ is as described herein. In some embodiments, L^{c} is —OC(O)N(R′)C(O)N(R′)S(O)₂— wherein R′ is as described herein. In some embodiments, L^{c} is or —OC(O)N(R′)S(O)₂— wherein R′ is as described herein. In some embodiments, L^{c} is —NHC(O)—. In some embodiments. In some embodiments, L^{c} is —NHC(S)—. In some embodiments, L^{c} is —NHC(NH)—. In some embodiments, L^{c} is —NHC(O)O—. In some embodiments, L^{c} is —NHC(NH)NH—. In some embodiments, L^{c} is —NHC(S)NHS(O)₂—. In some embodiments, L^{c} is —NHC(NH)NHS(O)₂—. In some embodiments, L^{c} is —NHC(O)C(O)NHS(O)₂—. In some embodiments, L^{c} is —NHC(O)NHS(O)₂NH—. In some embodiments, L^{c} is —NHS(O)₂—. In some embodiments, L^{c} is —OC(O)NH—. In some embodiments, L^{c} is —OC(O)NHC(O)NH—. In some embodiments, L^{c} is —OC(O)NHC(O)—. In some embodiments, L^{c} is —OC(O)NHC(O)NHS(O)₂—. In some embodiments, L^{c} is or —OC(O)NHS(O)₂—.

In some embodiments, L¹ is a covalent bond.

In some embodiments, L¹ is optionally substituted bivalent C₁₋₁₀ aliphatic. In some embodiments, L¹ is C₁₋₁₀ is optionally substituted alkylene. In some embodiments, L¹ is optionally substituted —(CH₂)₁₋₁₀— wherein each —CH₂— is independently optionally substituted. In some embodiments, L¹ is —(CH₂)m- wherein m is 0-6 (e.g., 0, 1, 2, 3, 4, 5 or 6) and each —CH₂— is independently optionally substituted. In some embodiments, L¹ is —CH₂—. In some embodiments, L¹ is —CH(COOH)—. In some embodiments, L¹ is —CH(CN)—.

In some embodiments, L¹ is —C(R′)₂—(CH₂)m-, wherein m is 0-6 (e.g., 0, 1, 2, 3, 4, 5 or 6), and each —CH₂— is independently optionally substituted. In some embodiments, L¹ is —CHR′—(CH₂)m-, wherein m is 0-6 (e.g., 0, 1, 2, 3, 4, 5 or 6), and each —CH₂— is independently optionally substituted. In some embodiments, L¹ is —CH(CH₃)—(CH₂)m-, wherein m is 0-6 (e.g., 0, 1, 2, 3, 4, 5 or 6), and each —CH₂— is independently optionally substituted. In some embodiments, L¹ is —CH(CH₃)—(CH₂)m-, wherein m is 0-6 (e.g., 0, 1, 2, 3, 4, 5 or 6).

In some embodiments, L¹ is —CH(CH₃)—(CH₂)m-CH(CN)—, wherein m is 0-6 (e.g., 0, 1, 2, 3, 4, 5 or 6), and each —CH₂— is independently optionally substituted. In some embodiments, L¹ is —CH(CH₃)—(CH₂)m-CH(CN)—, wherein m is 0-6 (e.g., 0, 1, 2, 3, 4, 5 or 6).

In some embodiments, L¹ is —C(R′)₂—(CH₂)m-C(R′)₂—, wherein m is 0-6 (e.g., 0, 1, 2, 3, 4, 5 or 6), and each —CH₂— is independently optionally substituted. In some embodiments, L¹ is —CH(R′)—(CH₂)m-C(R′)₂—, wherein m is 0-6 (e.g., 0, 1, 2, 3, 4, 5 or 6), and each —CH₂— is independently optionally substituted. In some embodiments, L¹ is —CH(CH₃)—(CH₂)m-C(R′)₂—, wherein m is 0-6 (e.g., 0, 1, 2, 3, 4, 5 or 6), and each —CH₂— is independently optionally substituted. In some embodiments, L¹ is —CH(CH₃)—(CH₂)m- C(R′)₂—, wherein m is 0-6 (e.g., 0, 1, 2, 3, 4, 5 or 6).

In some embodiments, a methylene unit is replaced with —C(R′)₂—. In some embodiments, a methylene unit is replaced with —O—. In some embodiments, a methylene unit is replaced with —C(O)—. In some embodiments, a methylene unit is replaced with —C(O)O—. In some embodiments, a methylene unit is replaced with —CH═CH—.

In some embodiments, L¹ is —C(R′)₂—(CH₂)m-C(O)—, wherein m is 0-6 (e.g., 0, 1, 2, 3, 4, 5 or 6), and each —CH₂— is independently optionally substituted. In some embodiments, L¹ is —CHR′—(CH₂)m-C(O)—, wherein m is 0-6 (e.g., 0, 1, 2, 3, 4, 5 or 6), and each —CH₂— is independently optionally substituted. In some embodiments, L¹ is —CH(CH₃)—(CH₂)m-C(O)—, wherein m is 0-6 (e.g., 0, 1, 2, 3, 4, 5 or 6), and each —CH₂— is independently optionally substituted. In some embodiments, L¹ is —CH(CH₃)—(CH₂)m-C(O)—, wherein m is 0-6 (e.g., 0, 1, 2, 3, 4, 5 or 6).

In some embodiments, L¹ is —C(R′)₂—(CH₂)m-C(O)O—, wherein m is 0-6 (e.g., 0, 1, 2, 3, 4, 5 or 6), and each —CH₂— is independently optionally substituted. In some embodiments, L¹ is —CHR′—(CH₂)m-C(O)O—, wherein m is 0-6 (e.g., 0, 1, 2, 3, 4, 5 or 6), and each —CH₂— is independently optionally substituted. In some embodiments, L¹ is —CH(CH₃)—(CH₂)m-C(O)O—, wherein m is 0-6 (e.g., 0, 1, 2, 3, 4, 5 or 6), and each —CH₂— is independently optionally substituted. In some embodiments, L¹ is —CH(CH₃)—(CH₂)m-C(O)O—, wherein m is 0-6 (e.g., 0, 1, 2, 3, 4, 5 or 6).

In some embodiments, L¹ is —CH(CH₃)—(CH₂)m-CH(C(O)OR)—, wherein m is 0-6 (e.g., 0, 1, 2, 3, 4, 5 or 6), and each —CH₂— is independently optionally substituted. In some embodiments, L¹ is —CH(CH₃)—(CH₂)m-CH(C(O)OR)—, wherein m is 0-6 (e.g., 0, 1, 2, 3, 4, 5 or 6). In some embodiments, R is —H. In some embodiments, R is not —H. In some embodiments, R is optionally substituted C₁₋₆ aliphatic. In some embodiments, R is methyl.

In some embodiments, L¹ is —C(R′)₂—(CH₂)m-CH═CH—, wherein m is 0-6 (e.g., 0, 1, 2, 3, 4, 5 or 6), and each —CH₂— and —CH═CH— is independently optionally substituted. In some embodiments, L′ is —CHR′—(CH₂)m-CH═CH—, wherein m is 0-6 (e.g., 0, 1, 2, 3, 4, 5 or 6), and each —CH₂— and —CH═CH— is independently optionally substituted. In some embodiments, L¹ is —CH(CH₃)—(CH₂)m-CH═CH—, wherein m is 0-6 (e.g., 0, 1, 2, 3, 4, 5 or 6), and each —CH₂— and —CH═CH— is independently optionally substituted. In some embodiments, L¹ is —CH(CH₃)—(CH₂)m-CH═CH—, wherein m is 0-6 (e.g., 0, 1, 2, 3, 4, 5 or 6).

In some embodiments, m is 0. In some embodiments, m is 1. In some embodiments, m is 2. In some embodiments, m is 3. In some embodiments, m is 4. In some embodiments, m is 5. In some embodiments, m is 5.

In some embodiments, L¹ is —CH(CH₃)—(CH₂)—. In some embodiments, L¹ is —CH(CH₃)—(CH₂)₂—. In some embodiments, L¹ is —CH(CH₃)—(CH₂)₃—. In some embodiments, L¹ is —CH(CH₃)—(CH₂)₄—.

In some embodiments, L¹ is —CH(CH₃)—CH(CH₃)—. In some embodiments, L¹ is —CH(CH₃)—(CH₂)—CH(CH₃)—. In some embodiments, L¹ is —CH(CH₃)—(CH₂)₂—CH(CH₃)—. In some embodiments, L¹ is —CH(CH₃)—(CH₂)₃—CH(CH₃)—.

In some embodiments, L¹ is —CH(CH₃)—C(CH₃)₂—. In some embodiments, L¹ is —CH(CH₃)—(CH₂)—C(CH₃)₂—. In some embodiments, L¹ is —CH(CH₃)—(CH₂)₂—C(CH₃)₂—. In some embodiments, L¹ is —CH(CH₃)—(CH₂)₃—C(CH₃)₂—.

In some embodiments, L¹ is —CH(CH₃)—(CH₂)₂—C(O)—. In some embodiments, L¹ is —CH(CH₃)—(CH₂)₂—C(O)O—. In some embodiments, L¹ is —CH(CH₃)—(CH₂)₃—C(O)—. In some embodiments, L¹ is —CH(CH₃)—(CH₂)₄—C(O)—.

In some embodiments, L¹ is —CH(CH₃)—(CH₂)₃—C(O)O—. In some embodiments, L¹ is —CH(CH₃)—(CH₂)₄—C(O)—. In some embodiments, L¹ is —CH(CH₃)—(CH₂)₄—C(O)O—.

As described herein, in some embodiments, —CH(CH₃)— is not bonded to R^{L} (i.e., the other end of L¹ is bonded to R^{L}). In some embodiments, —CH(CH₃)— is S. In some embodiments, —CH(CH₃)— is R.

In some embodiments, L¹ is L as described herein. In some embodiments, L¹ is L″ as described herein.

In some embodiments, L¹ is a covalent bond.

In some embodiments, L¹ is an optionally substituted bivalent C₁₋₆ aliphatic chain. In some embodiments, L¹ is a bivalent C₁₋₆ aliphatic chain. In some embodiments, L¹ is optionally substituted bivalent C₁₋₄ aliphatic. In some embodiments, L¹ is linear. In some embodiments, L¹ is branched. In some embodiments, L¹ is optionally substituted bivalent C₁₋₄ alkylene. In some embodiments, L¹ is optionally substituted —CH₂—. In some embodiments, L¹ is optionally substituted —(CH₂)₂—. In some embodiments, L¹ is optionally substituted —(CH₂)₃—. In some embodiments, L¹ is optionally substituted —(CH₂)₄—. In some embodiments, L¹ is —CH(CH₃)—(CH₂)m-, wherein m is 0, 1, 2, or 3. In some embodiments, m is 0. In some embodiments, m is 1. In some embodiments, m is 2. In some embodiments, m is 3.

In some embodiments, L¹ is an optionally substituted, bivalent C₁₋₁₅ (e.g., C₁₋₁₀, C₁₋₆, C₃₋₁₀, C₁, C₂, C₃, C₄, C₅, C₆, C₇, C₈, C₉, C₁₀, or C₁₁₋₁₅) aliphatic or heteroaliphatic group having 1-10 (e.g., 1, 2, 3, 4, 5, 6, 7, 8, 9, or 10) heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L¹ is an optionally substituted, bivalent C₁₋₁₅ aliphatic wherein one or more methylene units of the group are optionally and independently replaced as described herein. In some embodiments, L¹ is an optionally substituted, bivalent C₁₋₁₅ heteroaliphatic group having 1-10 heteroatoms wherein one or more methylene units of the group are optionally and independently replaced as described herein. In some embodiments, L¹ is an optionally substituted, bivalent C₁₋₁₅ alkylene wherein one or more methylene units of the group are optionally and independently replaced as described herein. In some embodiments, at least one methylene unit is replaced as described herein. In some embodiments, two or more methylene units are independently replaced as described herein. In some embodiments, at least one methylene unit is replaced with -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, a methylene unit is replaced with -Cy-. In some embodiments, it is replaced with —O—. In some embodiments, it is replaced with —S—. In some embodiments, it is replaced with —S—S—. In some embodiments, it is replaced with —N(R′)—. In some embodiments, it is replaced with —C(O)—. In some embodiments, it is replaced with —C(O)S—. In some embodiments, it is replaced with —C(O)O—. In some embodiments, it is replaced with —C(S)—. In some embodiments, it is replaced with —C(NR′)—. In some embodiments, it is replaced with —C(O)N(R′)—. In some embodiments, it is replaced with —C(NR′)N(R′)—. In some embodiments, it is replaced with —C(S)N(R′)—. In some embodiments, it is replaced with —N(R′)C(O)N(R′)—. In some embodiments, it is replaced with —N(R′)C(NR′)N(R′)—. In some embodiments, it is replaced with —N(R′)C(S)N(R′)—. In some embodiments, it is replaced with —N(R′)C(O)O—. In some embodiments, it is replaced with —N(R′)C(O)N(R′)S(O)₂—. In some embodiments, it is replaced with —OC(O)N(R′)—. In some embodiments, it is replaced with —OC(O)N(R′)S(O)₂—. In some embodiments, it is replaced with —S(O)—. In some embodiments, it is replaced with —S(O)₂—. In some embodiments, it is replaced with —S(O)₂N(R′)—. In some embodiments, it is replaced with —P(O)(OR′)—. In some embodiments, it is replaced with —P(O)(OR′)O—.

In some embodiments, L¹ is -L^{a}″-L^{b}″-L^{c}″- wherein each of L^{a}″, L^{b}″ and L^{c}″ id independently as described herein (e.g., see embodiments described in the section for certain R^{L} embodiments).

In some embodiments, L is L″ as described herein.

In some embodiments, L is a covalent bond.

In some embodiments, L is an optionally substituted bivalent C₁₋₆ aliphatic chain. In some embodiments, L is a bivalent C₁₋₆ aliphatic chain. In some embodiments, L is optionally substituted bivalent C₁₋₄ aliphatic. In some embodiments, L is linear. In some embodiments, L is branched. In some embodiments, L is optionally substituted bivalent C₁₋₄ alkylene. In some embodiments, L is optionally substituted —CH₂—. In some embodiments, L is optionally substituted —(CH₂)₂—. In some embodiments, L is optionally substituted —(CH₂)₃—. In some embodiments, L is optionally substituted —(CH₂)₄—. In some embodiments, L is —CH(CH₃)—(CH₂)m-, wherein m is 0, 1, 2, or 3. In some embodiments, m is 0. In some embodiments, m is 1. In some embodiments, m is 2. In some embodiments, m is 3.

In some embodiments, L is an optionally substituted, bivalent C₁₋₁₅ (e.g., C₁₋₁₀, C₁₋₆, C₃₋₁₀, C₁, C₂, C₃, C₄, C₅, C₆, C₇, C₈, C₉, C₁₀, or C₁₁₋₁₅) aliphatic or heteroaliphatic group having 1-10 (e.g., 1, 2, 3, 4, 5, 6, 7, 8, 9, or 10) heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L is an optionally substituted, bivalent C₁₋₁₅ aliphatic wherein one or more methylene units of the group are optionally and independently replaced as described herein. In some embodiments, L is an optionally substituted, bivalent C₁₋₁₅ heteroaliphatic group having 1-10 heteroatoms wherein one or more methylene units of the group are optionally and independently replaced as described herein. In some embodiments, L is an optionally substituted, bivalent C₁₋₁₅ alkylene wherein one or more methylene units of the group are optionally and independently replaced as described herein. In some embodiments, at least one methylene unit is replaced as described herein. In some embodiments, two or more methylene units are independently replaced as described herein. In some embodiments, at least one methylene unit is replaced with -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, a methylene unit is replaced with -Cy-. In some embodiments, it is replaced with —O—. In some embodiments, it is replaced with —S—. In some embodiments, it is replaced with —S—S—. In some embodiments, it is replaced with —N(R′)—. In some embodiments, it is replaced with —C(O)—. In some embodiments, it is replaced with —C(O)S—. In some embodiments, it is replaced with —C(O)O—. In some embodiments, it is replaced with —C(S)—. In some embodiments, it is replaced with —C(NR′)—. In some embodiments, it is replaced with —C(O)N(R′)—. In some embodiments, it is replaced with —C(NR′)N(R′)—. In some embodiments, it is replaced with —C(S)N(R′)—. In some embodiments, it is replaced with —N(R′)C(O)N(R′)—. In some embodiments, it is replaced with —N(R′)C(NR′)N(R′)—. In some embodiments, it is replaced with —N(R′)C(S)N(R′)—. In some embodiments, it is replaced with —N(R′)C(O)O—. In some embodiments, it is replaced with —N(R′)C(O)N(R′)S(O)₂—. In some embodiments, it is replaced with —OC(O)N(R′)—. In some embodiments, it is replaced with —OC(O)N(R′)S(O)₂—. In some embodiments, it is replaced with —S(O)—. In some embodiments, it is replaced with —S(O)₂—. In some embodiments, it is replaced with —S(O)₂N(R′)—. In some embodiments, it is replaced with —P(O)(OR′)—. In some embodiments, it is replaced with —P(O)(OR′)O—.

### R^{L}

In some embodiments, R^{L} is R^{s} as described herein. In some embodiments, R^{L} is R′. In some embodiments, R^{L} is R.

In some embodiments, R^{L} is —H. In some embodiments, R^{L} is halogen. In some embodiments, R^{L} is —CN. In some embodiments, R^{L} is —C(O)OR. In some embodiments, R is —H. In some embodiments, R is optionally substituted C₁₋₆ aliphatic. In some embodiments, R^{L} is —C(O)OH. In some embodiments, R^{L} is —C(O)OMe. In some embodiments, R^{L} is —C(O)OEt.

In some embodiments, R^{L} is optionally substituted C-io aliphatic. In some embodiments, R^{L} is methyl. In some embodiments, R^{L} is ethyl. In some embodiments, R^{L} is i-Pr. In some embodiments, R^{L} is n-Bu. In some embodiments, R^{L} is tBu. In some embodiments, R^{L} is 2-hydroxyl-t-butyl. In some embodiments, R^{L} is

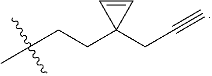 [SMILES: *CCC1(CC#C)C=C1]

In some embodiments, R^{L} is optionally substituted C₂₋₈ alkenyl. In some embodiments, R^{L} is optionally substituted C₂₋₈ alkynyl. In some embodiments, R^{L} is R^{s}, wherein R^{s} is C₁₋₁₀ cycloaliphatic. In some embodiments, R^{L} is optionally substituted C₃₋₈ cycloalkyl. In some embodiments, R^{L} is

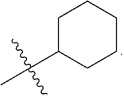 [SMILES: *C1CCCCC1]

In some embodiments, R^{L} is optionally substituted C₆₋₁₀ aryl. In some embodiments, R^{L} is optionally substituted phenyl. In some embodiments, R^{L} is optionally substituted C₆₋₁₅ arylaliphatic. In some embodiments, R^{L} is optionally substituted C₆₋₁₅ arylalkyl. In some embodiments, R^{L} is optionally substituted 3-12 membered heterocyclyl having 1-5 heteroatoms.

In some embodiments, R^{L} is R′ as described herein. In some embodiments, R′ is optionally substituted 5-14 (e.g., 5, 6, 9, 10, 14, etc.) membered heteroaryl having 1-5 (e.g., 1, 2, 3, 4, or 5) heteroatoms. In some embodiments, R′ is 5-membered heteroaryl having 1-5 heteroatoms. In some embodiments, R′ is 6-membered heteroaryl having 1-5 heteroatoms. In some embodiments, R′ is 9-membered heteroaryl having 1-5 heteroatoms. In some embodiments, R′ is 10-membered heteroaryl having 1-5 heteroatoms. In some embodiments, a heteroaryl ring has 1 heteroatom. In some embodiments, a heteroaryl ring has 2 heteroatoms. In some embodiments, a heteroaryl ring has 3 heteroatoms. In some embodiments, a heteroaryl ring has 4 heteroatoms. In some embodiments, a heteroatom is nitrogen. In some embodiments, a heteroatom is oxygen. In some embodiments, a heteroatom is sulfur. In some embodiments, R′ is optionally substituted thienyl. In some embodiments, R′ is thienyl. In some embodiments, R′ is optionally substituted 5-membered heteroaryl having 3 heteroatoms, wherein two of them are nitrogen and are bonded to each other, and the third is sulfur or nitrogen and is bonded to two carbon atoms. In some embodiments, R′ is optionally substituted

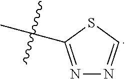 [SMILES: *c1nncs1]

In some embodiments, R′ is optionally substituted

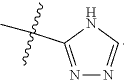 [SMILES: *c1nnc[nH]1]

In some embodiments, R′ is

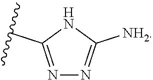 [SMILES: *c1nnc(N)[nH]1]

In some embodiments, R^{L} is selected from a group set forth below:

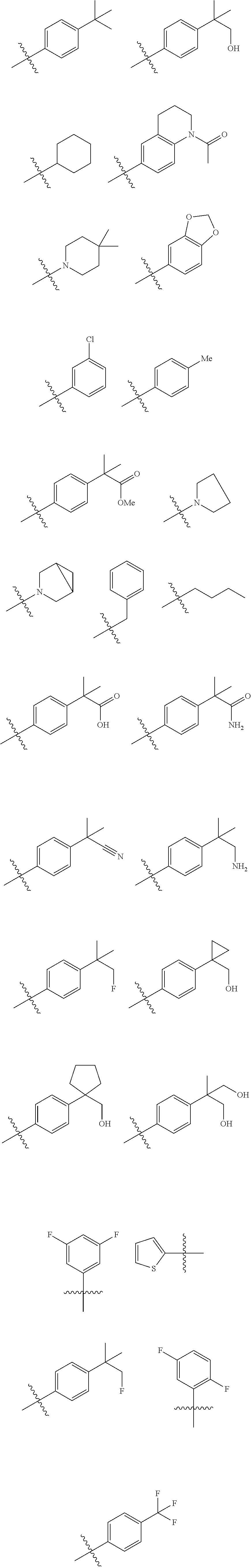

In some embodiments, R^{L} is —CH₂R′. In some embodiments, R^{L} is —CH₂C(O)R′. In some embodiments, R^{L} is —CH₂C(O)OR.

In some embodiments, R^{L} is —CH₂OR′. In some embodiments, R^{L} is —CH₂OR. In some embodiments, R is —H. In some embodiments, R is optionally substituted C₁₋₆ aliphatic.

In some embodiments, R^{L} is —CH═CHR′. In some embodiments, R^{L} is —CH═CHC(O)R′. In some embodiments, R^{L} is —CH═CHC(O)OR.

In some embodiments, R^{L} is —OR′, wherein R′ is as described herein. In some embodiments, R^{L} is —OR. In some embodiments, R^{L} is —OH. In some embodiments, R^{L} is —OMe.

In some embodiments, R^{L} is —N(R′)₂. In some embodiments, each R′ is independently optionally substituted C₁₋₁₀ aliphatic. In some embodiments, each R′ is independently methyl. In some embodiments, each R′ is independently ethyl. In some embodiments, each R′ is independently i-Pr. In some embodiments, each R′ is independently n-Bu. In some embodiments, each R′ is independently is tBu. In some embodiments, one of R′ is —H, and the other is optionally substituted C₁₋₁₀ aliphatic. In some embodiments, one of R′ is —H, and the other is methyl. In some embodiments, one of R′ is —H, and the other is ethyl. In some embodiments, one of R′ is —H, and the other is i-Pr. In some embodiments, one of R′ is —H, and the other is n-Bu. In some embodiments, one of R′ is —H, and the other is tBu. In some embodiments, two R′ are taken together with the nitrogen atom to form an optionally substituted, 3-15 membered, monocyclic, bicyclic or polycyclic ring having, in addition to the atom, 0-5 heteroatoms. In some embodiments, a formed ring is saturated. In some embodiments, R^{L} is

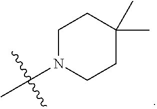 [SMILES: *N1CCC(C)(C)CC1]

In some embodiments, R^{L} is

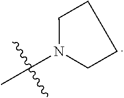 [SMILES: *N1CCCC1]

n some embodiments, R^{L} is

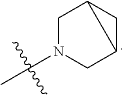 [SMILES: *N1CC2CC2C1]

In some embodiments, R^{L} is R^{s} as described herein. For example, in some embodiments, R^{L} is R^{s} wherein R^{s} is —S(O)₂R′ wherein R′ is as described herein. In some embodiments, R′ is optionally substituted C₁₋₁₀ aliphatic, optionally substituted C₆₋₁₄ aryl, optionally substituted C₆₋₁₅ arylaliphatic, optionally substituted 5-14 membered heteroaryl having 1-5 heteroatoms, or optionally substituted 3-15 membered heterocyclyl having 1-5 heteroatoms. In some embodiments, R′ is optionally substituted C₁₋₆ aliphatic. In some embodiments, R′ is optionally substituted cyclohexyl. In some embodiments, R′ is optionally substituted phenyl. In some embodiments, R′ is optionally substituted benzyl. In some embodiments, R′ is selected from a group set forth below:

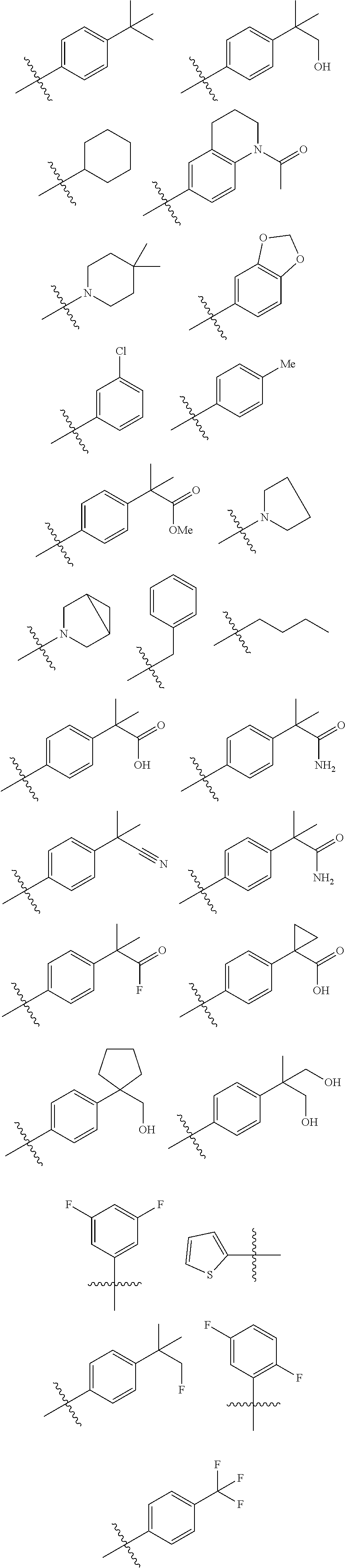

In some embodiments, R^{L} is —OS(O)₂OR′, wherein R′ is as described herein. In some embodiments, R^{L} is —OS(O)₂OH. In some embodiments, R^{L} is —OR. In some embodiments, R^{L} is —OS(O)₂R. In some embodiments, R is —H. In some embodiments, R is optionally substituted C₁₋₆ aliphatic. In some embodiments, R is optionally substituted phenyl. In some embodiments, R^{L} is protected hydroxyl.

In some embodiments, R^{L} is —C(O)R^{s} wherein R^{s} is as described herein. In some embodiments, R^{L} is —C(O)H. In some embodiments, R^{L} is —C(O)R, wherein R is as described herein. In some embodiments, R is —H or optionally substituted C₁₋₆ aliphatic. In some embodiments, R^{L} is —C(O)CH₃.

In some embodiments, R^{L} is —C(O)OR^{s} wherein R^{s} is as described herein. In some embodiments, R^{L} is —C(O)O-L″-R′ wherein each variable is independently as described herein. In some embodiments, L″ is a covalent bond. In some embodiments, R^{L} is —C(O)OR′, wherein R′ is as described herein. In some embodiments, R^{L} is —C(O)OH. In some embodiments, R^{L} is —C(O)OMe. In some embodiments, R^{L} is —C(O)OEt. In some embodiments, R^{L} is —C(O)Oi-Pr. In some embodiments, R^{L} is —C(O)On-Bu. In some embodiments, L″ is —C(O)O—. In some embodiments, R^{L} is —C(O)OC(O)OR′ wherein R is as described herein. In some embodiments, R′ is C₁₋₆ aliphatic. In some embodiments, R^{L} is —C(O)OC(O)O(i-Pr).

In some embodiments, R^{L} is —C(O)N(R^{s})₂, wherein each R^{s} is independently as described herein. In some embodiments, R^{L} is —C(O)N(R′)₂ wherein each R′ is independently as described herein. In some embodiments, R^{L} is —C(O)NH₂.

In some embodiments, R^{L} is —C(O)N(R′)(OR′). In some embodiments, R^{L} is —C(O)N(R′)(OR). In some embodiments, R′ is R as described herein. In some embodiments, R′ is —H. In some embodiments, R′ is optionally substituted C₁₋₆ aliphatic. In some embodiments, R′ is optionally substituted C₁₋₆ alkyl. In some embodiments, R′ is methyl. In some embodiments, R is —H. In some embodiments, R is C₁₋₆ aliphatic. In some embodiments, R is C₁₋₆ alkyl. In some embodiments, R is methyl. In some embodiments, —C(O)N(CH₃)OCH₃.

In some embodiments, R^{L} is —C(O)N(R′)S(O)₂R^{s}, wherein each of R′ and R^{s} is independently as described herein. In some embodiments, R′ is —H or optionally substituted C₁₋₆ aliphatic. In some embodiments, R^{L} is —C(O)NHS(O)₂R^{s}, wherein R^{s} is as described herein. In some embodiments, R^{s} is -L″-R′ wherein each of L″ and R is independently as described herein. In some embodiments, R^{s} is R′ as described herein. In some embodiments, R^{L} is —C(O)N(R′)S(O)₂R′, wherein each R′ is independently as described herein. In some embodiments, R^{L} is —C(O)N(R′)S(O)₂R′, wherein each R′ is independently selected from H or optionally substituted group C₁₋₁₀ aliphatic. In some embodiments, R^{L} is —C(O)N(R′)S(O)₂R′, wherein each R′ is independently selected from H or methyl. In some embodiments, R^{L} is —C(O)N(R′)S(O)₂R′, wherein each R′ is independently selected from H or ethyl. In some embodiments, R^{L} is —C(O)NHS(O)₂R′. In some embodiments, R^{L} is —C(O)N(R′)S(O)₂R′, wherein each R′ is independently selected from H or t-butyl. In some embodiments, R^{L} is —C(O)N(H)S(O)₂Me. In some embodiments, R^{L} is —C(O)N(H)S(O)₂Et. In some embodiments, R^{L} is —C(O)N(H)S(O)₂t-Bu.

In some embodiments, R^{L} is —C(O)N(R′)C(R′)₂C(O)N(R^{s})₂, wherein each variable is independently as described herein. In some embodiments, R^{L} is —C(O)N(R′)C(R′)₂S(O)₂R^{s}, wherein each variable is independently as described herein. In some embodiments, R^{L} is —C(O)N(R′)C(R′)₂S(O)₂N(R^{s})₂, wherein each variable is independently as described herein. In some embodiments, R^{L} is —C(O)N(R′)C(R′)₂P(O)(R^{s})₂, wherein each variable is independently as described herein. In some embodiments, R^{L} is —C(O)N(R′)C(R′)₂N(R′)C(O)N(R′)S(O)₂, wherein each variable is independently as described herein. In some embodiments, R^{L} is —C(O)N(R′)C(R^{s})₃, wherein each variable is independently as described herein. In some embodiments, R^{L} is —C(O)N(R′)C(R′)₂C(O)N(R′)S(O)₂R^{s}, wherein each variable is independently as described herein. In some embodiments, R^{L} is —C(O)N(R′)S(O)₂N(R^{s})₂, wherein each variable is independently as described herein. In some embodiments, R^{L} is —C(O)N(R′)C(NR′)N(R^{s})₂, wherein each variable is independently as described herein.

In some embodiments, R^{L} is —S(O)₂R^{s} wherein R^{s} is as described herein. In some embodiments, R^{L} is —S(O)₂N(R^{s})₂ wherein each R^{s} is independently as described herein. In some embodiments, R^{L} is —P(O)(R^{s})₂ wherein each R^{s} is independently as described herein. In some embodiments, R^{L} is —OS(O)₂R^{s} wherein R^{s} is as described herein. In some embodiments, R^{L} is —OS(O)₂OR^{s} wherein R^{s} is as described herein.

In some embodiments, R^{L} is —N(R^{s})₂ wherein each R^{s} is independently as described herein. In some embodiments, each R^{s} is independently R′ as described herein. In some embodiments, each R^{s} is independently R as described herein.

In some embodiments, R^{L} is —N(R′)C(O)R^{s}, wherein each of R′ and R^{s} is independently as described herein. In some embodiments, R^{L} is —NHC(O)R^{s}, wherein each of R′ and R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(O)R′ wherein R′ is optionally substituted C₁₋₆ aliphatic. In some embodiments, R′ is optionally substituted C₁₋₆ alkyl. In some embodiments, R^{L} is —N(R)C(O)R. In some embodiments, R^{L} is —NCO. In some embodiments, R^{s} is —N(R′)₂, wherein each R′ is independently as described herein. In some embodiments, R^{s} is —NHR′ wherein R′ is as described herein. In some embodiments, R^{s} is —NH₂. In some embodiments, R^{s} is —N(R′)S(O)₂R as described herein.

In some embodiments, R^{L} is —N(R′)C(S)R^{s}, wherein each of R′ and R^{s} is independently as described herein. In some embodiments, R^{L} is —NHC(S)R^{s}, wherein each of R′ and R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(S)R′ wherein R′ is optionally substituted C₁₋₆ aliphatic. In some embodiments, R′ is optionally substituted C₁₋₆ alkyl. In some embodiments, R^{L} is —N(R)C(S)R. In some embodiments, R^{L} is —NCS. In some embodiments, R^{s} is —N(R′)₂, wherein each R′ is independently as described herein. In some embodiments, R^{s} is —NHR′ wherein R′ is as described herein. In some embodiments, R^{s} is —NH₂. In some embodiments, R^{s} is —N(R′)S(O)₂R as described herein.

In some embodiments, R^{L} is —N(R′)C(NR′)R^{s}, wherein each of R′ and R^{s} is independently as described herein. In some embodiments, R^{L} is —N(R′)C(NR′)R^{s}, wherein each of R′ and R^{s} is independently as described herein. In some embodiments, R^{L} is —NHC(NR′)R^{s}, wherein each of R′ and R^{s} is independently as described herein. In some embodiments, R^{L} is —N(R′)C(NH)R^{s}, wherein each of R′ and R^{s} is independently as described herein. In some embodiments, R^{L} is —NHC(NH)R^{s}, wherein each of R′ and R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(S)R′ wherein R′ is optionally substituted C₁₋₆ aliphatic. In some embodiments, R′ is optionally substituted C₁₋₆ alkyl. In some embodiments, R^{L} is —N(R)C(S)R. In some embodiments, R^{L} is —NCS. In some embodiments, R^{s} is —N(R′)₂, wherein each R′ is independently as described herein. In some embodiments, R^{s} is —NHR′ wherein R′ is as described herein. In some embodiments, R^{s} is —NH₂. In some embodiments, R^{s} is —N(R′)S(O)₂R as described herein.

In some embodiments, R^{L} is —N(R′)C(O)OR^{s}, wherein each of R′ and R^{s} is independently as described herein. In some embodiments, R^{L} is —NHC(O)OR^{s}, wherein each of R′ and R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(O)OR′ wherein R′ is optionally substituted C₁₋₆ aliphatic. In some embodiments, R′ is optionally substituted C₁₋₆ alkyl. In some embodiments, R^{L} In some embodiments, —NHBoc.

In some embodiments, R^{L} is —N(R′)C(O)N(R^{s})₂, wherein each of R′ and R^{s} is independently as described herein. In some embodiments, R^{L} is —N(R′)C(O)N(R′)₂, wherein each R′ is independently as described herein. In some embodiments, R^{L} is —NHC(O)N(R^{s})₂, wherein each R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(O)N(R′)₂ wherein R′ is optionally substituted C₁₋₆ aliphatic. In some embodiments, R′ is optionally substituted C₁₋₆ alkyl.

In some embodiments, R^{L} is —N(R′)C(NR′)N(R^{s})₂, wherein each of R′ and R^{s} is independently as described herein. In some embodiments, R^{L} is —N(R′)C(NR′)N(R′)₂, wherein each R′ is independently as described herein. In some embodiments, R^{L} is —N(R′)C(NH)N(R′)₂, wherein each R′ is independently as described herein. In some embodiments, R^{L} is —NHC(NH)N(R^{s})₂, wherein each R^{s} is independently as described herein. In some embodiments, R^{s} is -L″-R′, wherein each of L″ and R′ is independently as described herein. In some embodiments, R^{s} is —R′ as described herein. In some embodiments, R^{s} is R as described herein. In some embodiments, each R′ is independently —H or C₁₋₆ aliphatic. In some embodiments, R^{L} is —NHC(O)N(R′)₂ wherein R′ is optionally substituted C₁₋₆ aliphatic. In some embodiments, R′ is optionally substituted C₁₋₆ alkyl.

In some embodiments, R^{L} is —N(R¹⁵)C(O)N(R¹⁶)S(O)₂R^{s}, wherein each of R¹⁵ and R¹⁶ is independently R′ as described herein, and R^{s} is independently as described herein. In some embodiments, R^{L} is —N(R′)C(O)N(R′)S(O)₂R^{s}, wherein each of R′ and R^{s} is independently as described herein. In some embodiments, each R′ is independently —H or C₁₋₆ aliphatic. In some embodiments, R^{L} is —NHC(O)N(R′)S(O)₂R^{s}, wherein each of R′ and R^{s} is independently as described herein. In some embodiments, R^{L} is —NHC(O)NHS(O)₂R^{s}, wherein R^{s} is as described herein.

In some embodiments, R^{s} is -L″-R′ wherein each of L″ and R′ is independently as described herein. In some embodiments, R^{s} is -Cy-C(R′)₂—C(R′)₂—R′, wherein each of -Cy- and R′ is independently as described herein. In some embodiments, R^{s} is -Cy-C(R′)₂—C(R′)₂—R, wherein each of -Cy-, R′ and R is independently as described herein. In some embodiments, R^{s} is -Cy-C(R′)₂—C(R′)₂—OH, wherein each of -Cy- and R′ is independently as described herein. In some embodiments, R^{s} is -Cy-C(R′)₂—C(R′)₂—OR′, wherein each of -Cy- and R′ is independently as described herein. In some embodiments, R^{s} is -Cy-C(R′)₂—C(R′)₂—OR, wherein each of -Cy-, R′ and R is independently as described herein. In some embodiments, R^{s} is -Cy-C(R′)₂—C(R′)₂—N(R′)—R′, wherein each of -Cy- and R′ is independently as described herein. In some embodiments, R^{s} is -Cy-O—R′, wherein each of -Cy- and R′ is independently as described herein. In some embodiments, R^{s} is -Cy-C(O)—R′, wherein each of -Cy- and R′ is independently as described herein. In some embodiments, R^{s} is -Cy-C(O)—C(R′)₂—R′, wherein each of -Cy- and R′ is independently as described herein. In some embodiments, R^{s} is -Cy-C(R′)₂—R′, wherein each of -Cy- and R′ is independently as described herein. In some embodiments, R^{s} is —C(R′)₂-Cy-R′, wherein each of -Cy- and R′ is independently as described herein. In some embodiments, R^{s} is —CH₂-Cy-R′, wherein —CH₂— is optionally substituted and each of R′ and -Cy- is as described herein. In some embodiments, R^{s} is —CH₂-Cy-R′, wherein each of R′ and -Cy- is as described herein. In some embodiments, R^{s} is -Cy-R′, wherein each of -Cy- and R′ is independently as described herein. In some embodiments, R^{s} is -Cy-C(R′)₂—C(O)O—R′, wherein each of -Cy- and R′ is independently as described herein. In some embodiments, R^{s} is -Cy-C(R′)₂—C(O)(N(R′)—R′, wherein each of-Cy- and R′ is independently as described herein. In some embodiments, each R′ is independently R. In some embodiments, —C(R′)₂— bonded to -Cy- is —C(CH₃)₂—. In some embodiments, —C(R′)₂-bonded to —C(R′)₂— that is bonded to -Cy- is optionally substituted —CH₂—. In some embodiments, it is —CH₂—. In some embodiments, —C(R′)₂— bonded to R′, —C(O)—, —C(O)O—, —C(O)N(R′)—, etc. is optionally substituted —CH₂—. In some embodiments, it is —CH₂—. In some embodiments, R^{s} is —N(R′)₂. In some embodiments, R^{s} is —N(R)₂, wherein each R is independently —H or C₁₋₆ aliphatic. In some embodiments, R^{s} is —NH₂. In some embodiments, R^{s} is R′ as described herein. In some embodiments, R^{s} is optionally substituted phenyl. In some embodiments, R^{s} is phenyl substituted with one or more halogen. In some embodiments, R^{s} is phenyl substituted with one or more —F. In some embodiments, R^{s} is 2, 5-difluorophenyl. In some embodiments, R^{s} is 3, 5-difluorophenyl. In some embodiments, R^{s} is optionally substituted 5-membered heteroaryl. In some embodiments, R^{s} is 2-thienyl. In some embodiments, R^{s} is optionally substituted 6-membered heteroaryl. In some embodiments, R^{L} is —N(R′)C(O)N(R′)S(O)₂R′, wherein each R′ is independently as described herein. In some embodiments, R^{L} is —N(R′)C(O)N(R′)S(O)₂R′, wherein each R′ is independently selected from H, optionally substituted C₁₋₁₀ aliphatic, optionally substituted C₆₋₁₄ aryl, optionally substituted C₆₋₁₅ arylaliphatic, optionally substituted 5-14 membered heteroaryl having 1-5 heteroatoms, or optionally substituted 3-15 membered heterocyclyl having 1-5 heteroatoms. In some embodiments, R^{L} is —N(R′)C(O)N(R′)S(O)₂R′, wherein each R′ is independently selected from H or optionally substituted C₁₋₆ aliphatic. In some embodiments, R^{L} is —N(R′)C(O)N(R′)S(O)₂R′, wherein each R′ is independently selected from H or optionally substituted cyclohexyl. In some embodiments, R^{L} is —N(R′)C(O)N(R′)S(O)₂R′, wherein each R′ is independently selected from H or optionally substituted phenyl. In some embodiments, R^{L} is —N(R′)C(O)N(R′)S(O)₂R′, wherein each R′ is independently selected from H or optionally substituted 5-14 membered heteroaryl having 1-5 heteroatoms. In some embodiments, R^{L} is —N(R′)C(O)N(R′)S(O)₂R′, wherein each R′ is independently selected from H or optionally substituted benzyl.

In some embodiments, L″ is -L^{a}″-L^{b}″-L^{c}″-, wherein L^{a}″ is a covalent bond, or an optionally substituted, bivalent C₁₋₃ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—; L^{b}″ is a covalent bond, or an optionally substituted, bivalent C₁₋₂ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—; and L^{c}″ is a covalent bond, or an optionally substituted, bivalent C₁ aliphatic group wherein a methylene unit of the group is optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—.

In some embodiments, L^{a}″ is -Cy-, L^{b}″ is optionally substituted —CH₂—CH₂—, and L^{c}″ is —O—, —S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L^{a}″ is -Cy-, L^{b}″ is optionally substituted —C(R′)₂—C(R′)₂—, and L^{c}″ is —O—, —S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L^{a}″ is -Cy-, L^{b}″ is optionally substituted —CH₂—, and L^{c}″ is —O—, —S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L^{a}″ is -Cy-, L^{b}″ is optionally substituted —C(R′)₂—, and L^{c}″ is —O—, —S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L^{c}″ is —O—, —S—, —N(R′)—, —C(O)—, —C(O)O—, —C(O)N(R′)—, —N(R′)C(O)N(R′)—, —N(R′)C(O)O—, —N(R′)C(O)N(R′)S(O)₂—, —OC(O)N(R′)—, —OC(O)N(R′)S(O)₂—, —S(O)₂—, or —S(O)₂N(R′)—.

### L^{a}″

In some embodiments, L^{a}″ is a covalent bond. In some embodiments, L^{a}″ is -Cy- as described herein. In some embodiments, -Cy- is 3-20, 2-15, 3-10, 4-20, 5-20, or 3, 4, 5, 6, 7, 8, 9, 10, 11, 12, 13, 14, 15, 16, 17, 18, 19, 20-membered. In some embodiments, -Cy- comprises one or more, e.g., 1, 2, 3, 4, or 5 ring heteroatoms, e.g., each independently selected from nitrogen, oxygen, sulfur, phosphorus, silicon, etc. In some embodiments, each ring heteroatom is independently selected from nitrogen, oxygen and sulfur. In some embodiments, a heteroatom is nitrogen. In some embodiments, a heteroatom is oxygen. In some embodiments, a heteroatom is sulfur. In some embodiments, -Cy- is monocyclic. In some embodiments, -Cy- is bicyclic. In some embodiments, -Cy- is polycyclic. In some embodiments, -Cy- is saturated. In some embodiments, -Cy- is partially unsaturated. In some embodiments, -Cy- is aromatic. In some embodiments, one or more monocyclic units of -Cy- are independently partially unsaturated. In some embodiments, one or more monocyclic units of -Cy- are independently aromatic. In some embodiments, each monocyclic unit of -Cy- is independently a 3-10 (e.g., 3-9, 3-8, 3-7, 4-7, 5-7, or 3, 4, 5, 6, 7, 8, 9, or 10) membered, saturated, partially unsaturated or aromatic ring having 0-5 heteroatoms. In some embodiments, each monocyclic aromatic ring unit is 5- or 6-membered. In some embodiments, -Cy- is optionally substituted phenylene. In some embodiments, -Cy- is optionally substituted 1,4-phenylene. In some embodiments, -Cy- is 1,4-phenylene. In some embodiments, -Cy- is 3-chloro-1,4-phenylene. In some embodiments, -Cy- is 3-chloro-1,2-phenylene. In some embodiments, -Cy- is optionally substituted 3-10 membered cycloalkylene. In some embodiments, -Cy- is optionally substituted 5-6 membered cycloalkylene. In some embodiments, -Cy- is an optionally substituted bicyclo[2.2.1]heptane ring. In some embodiments, -Cy- is an optionally substituted bivalent 5-membered heterocyclyl ring having 1, 2, 3 or 4 heteroatom. In some embodiments, -Cy- is an optionally substituted bivalent 6-membered heterocyclyl ring having 1, 2, 3 or 4 heteroatom. In some embodiments, a heterocyclyl ring is 5-membered. In some embodiments, it is 6-membered. In some embodiments, it has one heteroatom. In some embodiments, the heteroatom is nitrogen. In some embodiments, -Cy- is an optionally substituted bivalent pyrrolidine ring. In some embodiments, -Cy- is an optionally substituted bivalent piperidine ring. In some embodiments, -Cy- is an optionally substituted bivalent 3-azabicyclo[3.1.0]hexane ring.

In some embodiments, -Cy- is optionally substituted 6-membered cycloalkylene. In some embodiments, -Cy- is 6-membered cycloalkylene. In some embodiments, -Cy- is

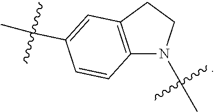 [SMILES: *c1ccc2c(c1)CCN2*]

In some embodiments, -Cy- is

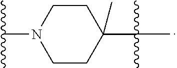 [SMILES: *N1CCC(*)(C)CC1]

In some embodiments, -Cy- is

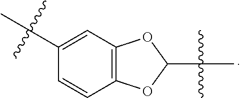 [SMILES: *c1ccc2c(c1)OC(*)O2]

In some embodiments, -Cy- is an optionally substituted 3-10 membered ring having 0-4 heteroatoms independently selected from nitrogen, oxygen and sulfur. In some embodiments, -Cy- is an optionally substituted 5-membered heteroaryl ring having 1-4 heteroatoms independently selected from nitrogen, oxygen and sulfur. In some embodiments, -Cy- is an optionally substituted 5-membered heteroaryl ring having 3 heteroatoms, wherein two of them are nitrogen and are bonded to each other, and the third is sulfur or nitrogen and is bonded to two carbon atoms. In some embodiments, -Cy- is

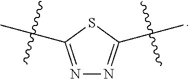 [SMILES: *c1nnc(*)s1]

In some embodiments, -Cy- is

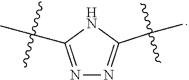 [SMILES: *c1nnc(*)[nH]1]

In some embodiments, L^{a}″ is —O—. In some embodiments, L^{a}″ is —C(O)—. In some embodiments, L^{a}″ is —OC(O)—. In some embodiments, L^{a}″ is —OC(O)N(R′)—. In some embodiments, L^{a}″ is —OC(O)NH—. In some embodiments, L^{a}″ is —N(R′)C(O)N(R′)—. In some embodiments, L^{a}″ is —NHC(O)NH—. In some embodiments, L^{a}″ is —OC(O)N(R′)S(O)₂—. In some embodiments, L^{a}″ is —OC(O)NHS(O)₂—. In some embodiments, L^{a}″ is —S(O)₂—. In some embodiments, L^{a}″ is —N(R′)S(O)₂—. In some embodiments, L^{a}″ is —NHS(O)₂—. In some embodiments, L^{a}″ is —C(O)N(R′)S(O)₂—. In some embodiments, L^{a}″ is —C(O)NHS(O)₂—. In some embodiments, L^{a}″ is —OC(O)N(R′)S(O)₂—. In some embodiments, L^{a}″ is —OC(O)NHS(O)₂—. In some embodiments, L^{a}″ is —N(R′)—. In some embodiments, L^{a}″ is —NH—. In some embodiments, L^{a}″ is —N(R′)C(O)N(R′)—. In some embodiments, L^{a}″ is —NHC(O)NH—. In some embodiments, L^{a}″ is —N(R′)C(O)N(R′)S(O)₂—. In some embodiments, L^{a}″ is —NHC(O)NHS(O)₂—. In some embodiments, —S(O)₂— is bonded to R^{L}. In some embodiments, —S(O)₂— is bonded to R₁. In some embodiments, each R′ is independently —H or C₁₋₆ aliphatic. In some embodiments, each R′ is independently —H. In some embodiments, two R′ are taken together with their intervening atoms to form an optionally substituted ring as described herein.

In some embodiments, L^{a}″ is —C(O)N(R′)S(O)₂— wherein R′ is as described herein. L^{a}″ is —C(O)N(R′)C(R′)₂C(O)N(R′)— wherein each R′ is independently as described herein. In some embodiments, L^{a}″ is —C(O)N(R′)C(R′)₂S(O)₂N(R′)— wherein each R′ is independently as described herein. In some embodiments, L^{a}″ is —C(O)N(R′)C(R′)₂P(O)(R′)— wherein each R′ is independently as described herein. In some embodiments, L^{a}″ is —C(O)N(R′)C(R′)₂N(R′)C(O)N(R′)S(O)₂— wherein each R′ is independently as described herein. In some embodiments, L^{a}″ is —C(O)N(R′)C(R′)₂— wherein each R′ is independently as described herein. In some embodiments, L^{a}″ is —C(O)N(R′)C(R′)₂C(O)N(R′)S(O)₂— wherein each R′ is independently as described herein. In some embodiments, L^{a}″ is —C(O)N(R′)S(O)₂N(R′)— wherein each R′ is independently as described herein. In some embodiments, L^{a}″ is —C(O)N(R′)C(NR′)N(R′)— wherein each R′ is independently as described herein. In some embodiments, L^{a}″ is —C(O)NHS(O)₂—. In some embodiments, L^{a}″ is —C(O)NHCH₂C(O)NH—. In some embodiments, L^{a}″ is —C(O)NHCH₂S(O)₂NH—. In some embodiments, L^{a}″ is —C(O)NHCH₂P(O)(R′)—. In some embodiments, L^{a}″ is —C(O)NHCH₂NHC(O)NHS(O)₂—. In some embodiments, L^{a}″ is —C(O)NHCH₂—. In some embodiments, L^{a}″ is —C(O)NHCH₂C(O)NHS(O)₂—. In some embodiments, L^{a}″ is —C(O)NHS(O)₂NH—. In some embodiments, L^{a}″ is —C(O)NHC(NR′)NH—.

In some embodiments, L^{a}″ is —P(O)(R′)— wherein R′ is as described herein. In some embodiments, L^{a}″ is —OS(O)₂O—. In some embodiments, L^{a}″ is —N(R′)C(O)— wherein R′ is as described herein. In some embodiments, L^{a}″ is —N(R′)C(S)— wherein R′ is as described herein. In some embodiments, L^{a}″ is —N(R′)C(NR′)— wherein each R′ is independently as described herein. In some embodiments, L^{a}″ is —N(R′)C(O)O— wherein R′ is as described herein. In some embodiments, L^{a}″ is —N(R′)C(NR′)N(R′)— wherein each R′ is independently as described herein. In some embodiments, L^{a}″ is —N(R′)C(S)N(R′)S(O)₂— wherein each R′ is independently as described herein. In some embodiments, L^{a}″ is —N(R′)C(NR′)N(R′)S(O)₂— wherein each R′ is independently as described herein. In some embodiments, L^{a}″ is —N(R′)C(O)C(O)N(R′)S(O)₂— wherein each R′ is independently as described herein. In some embodiments, L^{a}″ is —N(R′)C(O)N(R′)S(O)₂N(R′)— wherein each R′ is independently as described herein. In some embodiments, L^{a}″ is —N(R′)S(O)₂— wherein R′ is as described herein. In some embodiments, L^{a}″ is —OC(O)N(R′)— wherein R′ is as described herein. In some embodiments, L^{a}″ is —OC(O)N(R′)C(O)N(R′)— wherein each R′ is independently as described herein. In some embodiments, L^{a}″ is —OC(O)N(R′)C(O)— wherein R′ is as described herein. In some embodiments, L^{a}″ is —OC(O)N(R′)C(O)N(R′)S(O)₂— wherein R′ is as described herein. In some embodiments, L^{a}″ is or —OC(O)N(R′)S(O)₂— wherein R′ is as described herein. In some embodiments, L^{a}″ is —NHC(O)—. In some embodiments. In some embodiments, L^{a}″ is —NHC(S)—. In some embodiments, L^{a}″ is —NHC(NH)—. In some embodiments, L^{a}″ is —NHC(O)O—. In some embodiments, L^{a}″ is —NHC(NH)NH—. In some embodiments, L^{a}″ is —NHC(S)NHS(O)₂—. In some embodiments, L^{a}″ is —NHC(NH)NHS(O)₂—. In some embodiments, L^{a}″ is —NHC(O)C(O)NHS(O)₂—. In some embodiments, L^{a}″ is —NHC(O)NHS(O)₂NH—. In some embodiments, L^{a}″ is —NHS(O)₂—. In some embodiments, L^{a}″ is —OC(O)NH—. In some embodiments, L^{a}″ is —OC(O)NHC(O)NH—. In some embodiments, L^{a}″ is —OC(O)NHC(O)—. In some embodiments, L^{a}″ is —OC(O)NHC(O)NHS(O)₂—. In some embodiments, L^{a}″ is or —OC(O)NHS(O)₂—.

### L^{b}″

In some embodiments, L^{b}″ is a covalent bond. In some embodiments, L^{b}″ is optionally substituted —CH₂—. In some embodiments, L^{b}″ is optionally substituted —CH₂—CH₂—. In some embodiments, L^{b}″ is —C(R′)₂—. In some embodiments, L^{b}″ is —C(R′)₂—C(R′)₂—. In some embodiments, L^{b}″ is —C(R′)₂—CH₂—. In some embodiments, L^{b}″ is —C(CH₃)₂—. In some embodiments, L^{b}″ is —C(CH₃)(CN)—. In some embodiments, L^{b}″ is —C(CH₃)(CH₂OH)—. In some embodiments, L^{b}″ is —C(CH₂OH)₂—. In some embodiments, L^{b}″ is —C(CH₃)₂—CH₂—. In some embodiments, L^{b}″ is —C(CH₃)(CN)—CH₂—. In some embodiments, L^{b}″ is —C(O)—. In some embodiments, L^{b}″ is —CF₂—. In some embodiments, two R′ of —C(R′)₂— are taken together with the carbon atom to which they are attached to form an optionally substituted ring as described herein. In some embodiments, a formed ring is an optionally substituted 3-10 (e.g., 3, 4, 5, 6, 7, 8, 9, or 10) ring having 0-5 heteroatoms. In some embodiments, a formed ring is an optionally substituted 3-10 (e.g., 3, 4, 5, 6, 7, 8, 9, or 10) cycloaliphatic ring. In some embodiments, a formed ring is an optionally substituted cyclopropyl ring. In some embodiments, a formed ring is an optionally substituted cyclobutyl ring. In some embodiments, a formed ring is an optionally substituted cyclopentyl ring. In some embodiments, a formed ring is an optionally substituted cyclohexyl ring. In some embodiments, a formed ring is a monocyclic. In some embodiments, a formed ring is bicyclic. In some embodiments, a formed ring is polycyclic. In some embodiments, a formed ring is saturated. In some embodiments, a formed ring is partially unsaturated. In some embodiments, a formed ring is aromatic. In some embodiments, one or more monocyclic unit is independently partially unsaturated or aromatic. In some embodiments, a formed ring is an optionally substituted bicyclo[2.2.1]heptane ring. In some embodiments, L^{b}″ is —N(R′)— wherein R′ is as described herein. In some embodiments, L^{b}″ is —NH—. In some embodiments, L^{b}″ is —N(R′)— wherein R′ is optionally substituted C₁₋₆ aliphatic. In some embodiments, L^{b}″ is —S(O)₂—. In some embodiments, L^{b}″ is —S(O)₂N(R′)— wherein R′ is as described herein. In some embodiments, L^{b}″ is —S(O)₂NH—.

In some embodiments, L^{b}″ is -Cy- as described herein. In some embodiments, L^{b}″ is -Cy-CH₂— wherein -Cy- is as described herein and the —CH₂— is optionally substituted. In some embodiments, L^{b}″ is -Cy-C(R′)₂— wherein each of -Cy- and R′ is independently as described herein. In some embodiments, -Cy- is or comprises an aromatic ring having 0-4 heteroatoms. In some embodiments, -Cy-is monocyclic. In some embodiments, -Cy- is bicyclic. In some embodiments, -Cy- is polycyclic. In some embodiments, one monocyclic unit is a phenyl ring. In some embodiments, one monocyclic unit is a 5-6 membered heteroaryl having 1-4 heteroatoms independently nitrogen, oxygen and sulfur. In some embodiments, one monocyclic unit is a 5-membered heteroaryl having 1-4 heteroatoms independently nitrogen, oxygen and sulfur. In some embodiments, one monocyclic unit is a 6-membered heteroaryl having 1-4 heteroatoms independently nitrogen, oxygen and sulfur. In some embodiments, a monocyclic unit is a 3-10 membered saturated ring having 0-4 heteroatoms independently selected from nitrogen, oxygen and sulfur. In some embodiments, a monocyclic unit is a 3-10 membered partially unsaturated ring having 0-4 heteroatoms independently selected from nitrogen, oxygen and sulfur. In some embodiments, each monocyclic unit is independently 3-10 membered, is independently aromatic, saturated or partially unsaturated, and has 0-4 heteroatoms (e.g., 0, 1, 2, 3, or 4, independently selected nitrogen, oxygen and sulfur., etc.). In some embodiments, -Cy- is optionally substituted phenylene. In some embodiments, -Cy-is optionally substituted 1,2-phenylene. In some embodiments, -Cy- is optionally substituted 1,3-phenylene. In some embodiments, -Cy- is optionally substituted 1,4-phenylene. In some embodiments, -Cy-is optionally substituted bivalent pyridinyl ring. In some embodiments, -Cy- is optionally substituted

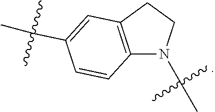 [SMILES: *c1ccc2c(c1)CCN2*]

in some embodiments, -Cy- is optionally substituted

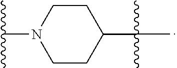 [SMILES: *C1CCN(*)CC1]

In some embodiments, -Cy- is

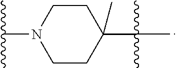 [SMILES: *N1CCC(*)(C)CC1]

In some embodiments, -Cy- is optionally substituted

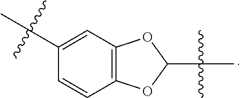 [SMILES: *c1ccc2c(c1)OC(*)O2]

In some embodiments, -Cy- is optionally substituted

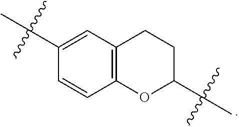 [SMILES: *c1ccc2c(c1)CCC(*)O2]

In some embodiments, -Cy- is bonded to L^{a}″.

### L^{c}″

In some embodiments, L^{c}″ is a covalent bond. In some embodiments, L^{c}″ is optionally substituted —CH₂—. In some embodiments, L^{c}″ is optionally substituted —CH₂—CH₂—. In some embodiments, L^{c}″ is —C(R′)₂—. In some embodiments, L^{c}″ is —C(R′)₂—C(R′)₂—. In some embodiments, L^{c}″ is —C(R′)₂—CH₂—. In some embodiments, L^{c}″ is —C(CH₃)₂—. In some embodiments, L^{c}″ is —C(CH₃)₂—CH₂—. In some embodiments, L^{c}″ is —O—. In some embodiments, L^{c}″ is —C(O)—. In some embodiments, L^{c}″ is —C(O)O—. In some embodiments, L^{c}″ is —N(R′)—. In some embodiments, L^{c}″ is —N(R′)— wherein R′ is C₁₋₆ aliphatic. In some embodiments, L^{c}″ is —N(R′)— wherein R′ is C₁₋₆ alkyl. In some embodiments, L^{c}″ is —NH—. In some embodiments, L^{c}″ is —N(CH₃)—. In some embodiments, L^{c}″ is —C(O)N(R′)—. In some embodiments, L^{c}″ is —C(O)N(R′)— wherein R′ is C₁₋₆ aliphatic. In some embodiments, L^{c}″ is —C(O)N(R′)— wherein R′ is C₁₋₆ alkyl. In some embodiments, L^{c}″ is —C(O)NH—. In some embodiments, L^{c}″ is —C(O)N(CH₃)—. In some embodiments, L^{c}″ is —S(O)₂—. In some embodiments, L^{c}″ is -Cy- is as described herein.

In some embodiments, L^{c}″ is optionally substituted —CH₂—. In some embodiments, L^{c}″ is —C(R′)₂—. In some embodiments, L^{c}″ is —CHR′—.

In some embodiments, L^{c}″ is -Cy- as described herein, e.g., embodiments described in the sections of L^{a}″, L^{b}″ etc. For example, in some embodiments, -Cy- is optionally substituted

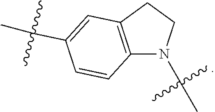 [SMILES: *c1ccc2c(c1)CCN2*]

In some embodiments, -Cy- is optionally substituted

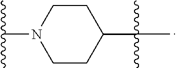 [SMILES: *C1CCN(*)CC1]

In some embodiments, -Cy- is

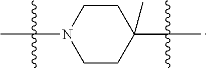 [SMILES: *N1CCC(*)(C)CC1]

In some embodiments, -Cy- is optionally substituted

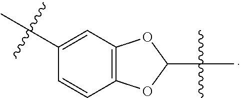 [SMILES: *c1ccc2c(c1)OC(*)O2]

In some embodiments, -Cy- is optionally substituted

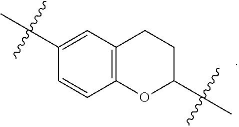 [SMILES: *c1ccc2c(c1)CCC(*)O2]

In some embodiments, -Cy- is bonded to L^{a}″.

In some embodiments, L″ is a covalent bond.

In some embodiments, L″ is -Cy- as described herein. In some embodiments, L″ is -Cy-N(R′)— wherein each variable is independently as described herein and —N(R′)— is bonded to R^{L}. In some embodiments, L″ is -Cy-N(R′)S(O)₂— wherein each variable is independently as described herein and —S(O)₂— is bonded to R^{L}. In some embodiments, L″ is -Cy-NHS(O)₂— wherein each variable is independently as described herein and —S(O)₂— is bonded to R^{L}. In some embodiments, L″ is -Cy-N(R′)S(O)₂-Cy- wherein each variable is independently as described herein. In some embodiments, L″ is -Cy-NHS(O)₂-Cy- wherein each variable is independently as described herein.

In some embodiments, R′ is —H. In some embodiments, R′ is C₁₋₆ aliphatic. In some embodiments, -Cy- is an optionally substituted 3-10 membered ring having 0-4 heteroatoms independently selected from nitrogen, oxygen and sulfur. In some embodiments, -Cy- is an optionally substituted 5-membered heteroaryl ring having 1-4 heteroatoms independently selected from nitrogen, oxygen and sulfur. In some embodiments, -Cy- is an optionally substituted 5-membered heteroaryl ring having 3 heteroatoms, wherein two of them are nitrogen and are bonded to each other, and the third is sulfur or nitrogen and is bonded to two carbon atoms. In some embodiments, -Cy- is

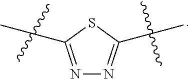 [SMILES: *c1nnc(*)s1]

In some embodiments, -Cy- is

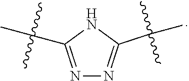 [SMILES: *c1nnc(*)[nH]1]

In some embodiments, L″ is

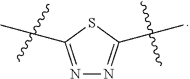 [SMILES: *c1nnc(*)s1]

In some embodiments, L″ is

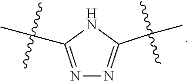 [SMILES: *c1nnc(*)[nH]1]

In some embodiments, L″ is

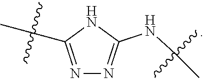 [SMILES: *Nc1nnc(*)[nH]1]

In some embodiments, L″ is

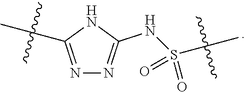 [SMILES: *c1nnc(NS(*)(=O)=O)[nH]1]

In some embodiments, R^{s} is -Cy-R′ wherein each variable is independently as described herein. For example, in some embodiments, -Cy- is

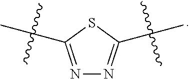 [SMILES: *c1nnc(*)s1]

In some embodiments, R′ is C₁₋₆ aliphatic (e.g., isopropyl). In some embodiments, R′ is —S(O)₂R wherein R is as described herein. In some embodiments, R is not —H. In some embodiments, R is optionally substituted aryl. In some embodiments, R^{s} is

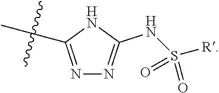

In some embodiments, R′ is not —H.

In some embodiments, R′ is —H. In some embodiments, R′ is R as described herein. In some embodiments, R′ is optionally substituted C₁₋₁₀ aliphatic. In some embodiments, R′ is optionally substituted C₁₋₆ aliphatic. In some embodiments, R′ is optionally substituted alkyl. In some embodiments, R′ is optionally substituted methyl. In some embodiments, R′ is methyl. In some embodiments, R′ is —CH₂OH. In some embodiments, R′ is —CF₃. In some embodiments, R′ is optionally substituted ethyl. In some embodiments, R′ is optionally substituted propyl. In some embodiments, R′ is optionally substituted butyl. In some embodiments, R′ is n-butyl. In some embodiments, R′ is t-butyl. In some embodiments, R′ is —C(CH₃)₂CH₂OH. In some embodiments, R′ is —C(CH₃)₂CH₂NH₂. In some embodiments, R′ is benzyl. In some embodiments, R′ is optionally substituted C₁₋₁₀ cycloaliphatic. In some embodiments, R′ is optionally substituted C₁₋₁₀ cycloalkyl. In some embodiments, R′ is optionally substituted cyclopropyl. In some embodiments, R′ is 1-hydroxylmethylcyclopropyl. In some embodiments, R′ is optionally substituted butyl. In some embodiments, R′ is optionally substituted pentyl. In some embodiments, R′ is 1-hydroxylmethylcyclopentyl. In some embodiments, R′ is optionally substituted cyclohexyl. In some embodiments, R′ is cyclohexyl. In some embodiments, R′ is

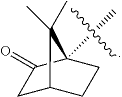

In some embodiments, R′ is

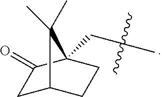 [SMILES: *C[C@]12CCC(CC1=O)C2(C)C]

In some embodiments, R′ is optionally substituted C₆ or C₁₋₁₀ aryl. In some embodiments, R′ is optionally substituted phenyl. In some embodiments, R′ is phenyl. In some embodiments, R′ is 4-t-butylphenyl. In some embodiments, R′ is 3-chlorophenyl. In some embodiments, R′ is 4-methylphenyl. In some embodiments, R′ is 4-trifluoromethylphenyl. In some embodiments, R′ is 2,5-difluorophenyl. In some embodiments, R′ is 3,5-difluorophenyl. In some embodiments, R′ is 2-trifluoromethoxy-4-bromophenyl.

In some embodiments, R′ is optionally substituted 5-14 (e.g., 5, 6, 9, 10, 14, etc.) membered heteroaryl having 1-5 heteroatoms. In some embodiments, R′ is 5-membered heteroaryl having 1-5 heteroatoms. In some embodiments, R′ is 6-membered heteroaryl having 1-5 heteroatoms. In some embodiments, R′ is 9-membered heteroaryl having 1-5 heteroatoms. In some embodiments, R′ is 10-membered heteroaryl having 1-5 heteroatoms. In some embodiments, a heteroaryl ring has 1 heteroatom. In some embodiments, a heteroaryl ring has 2 heteroatoms. In some embodiments, a heteroaryl ring has 3 heteroatoms. In some embodiments, a heteroaryl ring has 4 heteroatoms. In some embodiments, a heteroatom is nitrogen. In some embodiments, a heteroatom is oxygen. In some embodiments, a heteroatom is sulfur. In some embodiments, R′ is optionally substituted thienyl. In some embodiments, R′ is thienyl. In some embodiments, R′ is optionally substituted 5-membered heteroaryl having 3 heteroatoms, wherein two of them are nitrogen and are bonded to each other, and the third is sulfur or nitrogen and is bonded to two carbon atoms. In some embodiments, R′ is optionally substituted

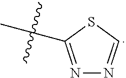 [SMILES: *c1nncs1]

In some embodiments, R′ is optionally substituted

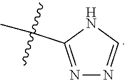 [SMILES: *c1nnc[nH]1]

In some embodiments, R′ is

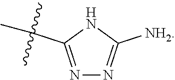 [SMILES: *c1nnc(N)[nH]1]

In some embodiments, R′ is optionally substituted C₁₋₁₀ heteroaliphatic having 1-5 (e.g., 1, 2, 3, 4, or 5) heteroatoms. In some embodiments, R′ is optionally substituted 3-15 membered heterocyclyl having 1-5 heteroatoms. In some embodiments, R′ is optionally substituted 3-membered heterocyclyl having one heteroatom. In some embodiments, R′ is optionally substituted 4-membered heterocyclyl having 1-2 heteroatoms. In some embodiments, R′ is optionally substituted 5-membered heterocyclyl having 1-4 heteroatoms. In some embodiments, R′ is optionally substituted 6-membered heterocyclyl having 1-5 heteroatoms. In some embodiments, R′ is optionally substituted 7-membered heterocyclyl having 1-5 heteroatoms. In some embodiments, R′ is optionally substituted 3-In some embodiments, R′ is optionally substituted pyrrolidinyl. In some embodiments, R′ is pyrrolidinyl. In some embodiments, R′ is optionally substituted piperidinyl. In some embodiments, R′ is 4,4-dimethyl-1-piperidinyl. In some embodiments, R′ is optionally substituted azabicyclo[3.1.0]hexyl. In some embodiments, R′ is 3-azabicyclo[3.1.0]hexyl.

In some embodiments, R′ is optionally substituted

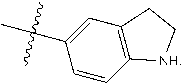 [SMILES: *c1ccc2c(c1)CCN2]

In some embodiments, R′ is

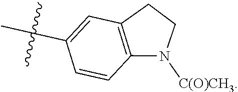

In some embodiments, R′ is optionally substituted

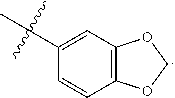 [SMILES: *c1ccc2c(c1)OCO2]

In some embodiments, R′ is

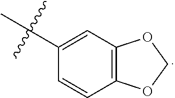 [SMILES: *c1ccc2c(c1)OCO2]

In some embodiments, R′ is optionally substituted C₆₋₁₄ aryl-C₁₋₁₀ aliphatic. In some embodiments, R′ is optionally substituted C₁₋₁₀ aliphatic-C₆₋₁₄ aryl. In some embodiments, R′ is optionally substituted C₁₋₁₀ alkyl-C₆₋₁₄ aryl. In some embodiments, R′ is optionally substituted C₃₋₁₀ cycloaliphatic-C₆₋₁₄ aryl. In some embodiments, R′ is optionally substituted C₃₋₁₀ cycloalkyl-C₆₋₁₄ aryl. For example, in some embodiments, R′ is 4-t-butylphenyl. In some embodiments, R′ is (2-hydroxyl-1,1-dimethylethyl)phenyl. In some embodiments, R′ is benzyl. In some embodiments, R′ is 4-methylphenyl. In some embodiments, R′ is optionally substituted cyclopropylphenyl. In some embodiments, R′ is optionally substituted cyclopentylphenyl. In some embodiments, R′ is optionally substituted t-butylphenyl. In some embodiments, R′ is 4-(1,1-dimethyl-2-fluoroethyl)phenyl.

Additional embodiments of R′ are described herein as examples.

In some embodiments, R^{s} is —N(R′)₂ wherein each R′ is independently as described herein. In some embodiments, each R′ is independently R as described herein. In some embodiments, each R′ is independently —H or C₁₋₆ aliphatic. In some embodiments, two R′ are taken together with the nitrogen to which they are attached to for an optionally substituted ring as described herein, e.g., an optionally substituted 3-10 (e.g., 3, 4, 5, 6, 7, 8, 9 or 10) membered ring having 0-3 heteroatoms to the nitrogen atom. In some embodiments, a formed ring is an optionally substituted 3-10 (e.g., 3, 4, 5, 6, 7, 8, 9 or 10) membered ring having 0 heteroatoms to the nitrogen atom. In some embodiments, a formed ring is saturated. In some embodiments, a formed ring is monocyclic. In some embodiments, R^{s} is —NH₂. In some embodiments, R^{s} is protected —NH₂. In some embodiments, R^{s} is —NHR wherein R is as described herein. In some embodiments, R^{s} is —NHR′ as described herein. In some embodiments, R^{s} is —N(R′)C(O)R or —NHC(O)R wherein each of R′ and R is independently as described herein. In some embodiments, R^{s} is —N(R′)C(O)OR wherein each of R′ and R is independently as described herein. In some embodiments, R^{s} is —NHC(O)OR wherein R is as described herein. In some embodiments, R is —H. In some embodiments, R is C₁₋₆ aliphatic. In some embodiments, R^{s} is —NHBoc.

In some embodiments, R^{L} is —N(H)C(O)N(R′)S(O)₂R^{s}, wherein R^{s} is selected from a group set forth below. In some embodiments, R^{L} is —N(H)C(O)N(R′)S(O)₂R^{s}, wherein R′ is —H or optionally substituted C₁₋₆ aliphatic and R^{s} is selected from a group set forth below. In some embodiments, R^{L} is —N(H)C(O)N(H)S(O)₂R^{s}, wherein R^{s} is selected from a group set forth below. In some embodiments, R^{L} is —N(H)C(O)N(H)S(O)₂R′, wherein R′ is selected from a group set forth below.

Certain embodiments for R^{s}, R₁ and R′ are described below:

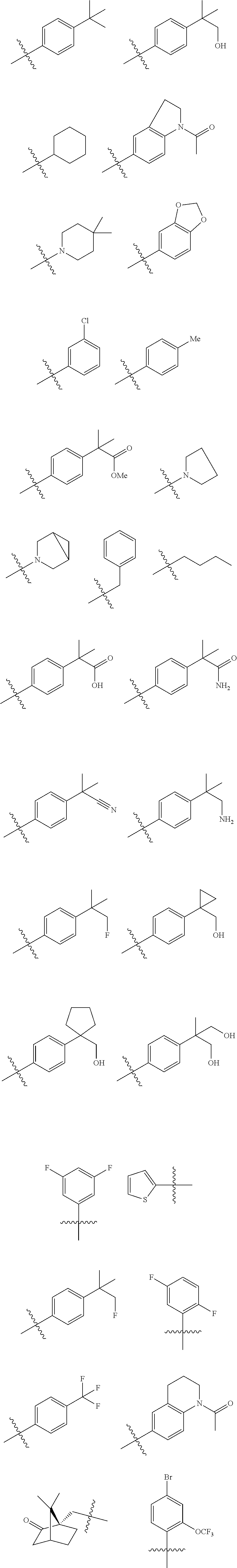

In some embodiments, R^{L} is —N(R′)C(O)N(R′)S(O)₂R^{s}, wherein R^{s} is —N(R′)₂, wherein each R′ is independently as described herein. In some embodiments, R^{L} is —N(R′)C(O)N(R′)S(O)₂N(R′)₂, wherein each R′ is independently as described herein. In some embodiments, R^{L} is —N(R′)C(O)N(R′)S(O)₂N(R′)₂, wherein each R′ is independently selected from H or optionally substituted C₁₋₁₀ aliphatic. In some embodiments, R^{L} is —N(R′)C(O)N(R′)S(O)₂N(R′)₂, wherein each R′ is independently selected from H or methyl. In some embodiments, R^{L} is —N(R′)C(O)N(R′)S(O)₂N(R′)₂, wherein each R′ is independently selected from H or ethyl. In some embodiments, R^{L} is —N(R′)C(O)N(R′)S(O)₂N(R′)₂, wherein each R′ is independently selected from H or isopropyl. In some embodiments, R^{L} is —N(R′)C(O)N(R′)S(O)₂N(R′)₂, wherein each R′ is independently selected from H or n-butyl. In some embodiments, R^{L} is —N(R′)C(O)N(R′)S(O)₂N(R′)₂, wherein each R′ is independently selected from H or t-butyl. In some embodiments, R^{L} is —N(H)C(O)N(H)S(O)₂N(R′)₂, wherein each R′ is independently as described herein. In some embodiments, R^{L} is —N(H)C(O)N(H)S(O)₂N(R′)₂, wherein each R′ is methyl. In some embodiments, R^{L} is —N(H)C(O)N(H)S(O)₂N(R′)₂, wherein each R′ is ethyl. In some embodiments, R^{L} is —N(H)C(O)N(H)S(O)₂N(R′)₂, wherein each R′ is isopropyl. In some embodiments, R^{L} is —N(H)C(O)N(H)S(O)₂N(R′)₂, wherein each R′ is n-butyl. In some embodiments, R^{L} is —N(H)C(O)N(H)S(O)₂N(R′)₂, wherein each R′ is t-butyl. In some embodiments, R^{L} is —N(H)C(O)N(H)S(O)₂N(R′)₂, wherein two R′ groups are taken together with the nitrogen atom to form an optionally substituted, 3-15 membered, monocyclic, bicyclic or polycyclic ring having, in addition to the atom, 0-5 heteroatoms. In some embodiments, R^{L} is —N(H)C(O)N(H)S(O)₂N(R′)₂, wherein two R′ groups are taken together with the nitrogen atom to form an optionally substituted, 3-10 membered, monocyclic, ring having, in addition to the atom, 1-3 heteroatoms. In some embodiments, R^{L} is —N(H)C(O)N(H)S(O)₂N(R′)₂, wherein two R′ groups are taken together with the nitrogen atom to form an optionally substituted group selected from

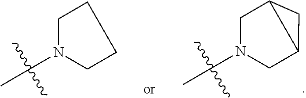 [SMILES: *N1CCCC1 ; *N1CC2CC2C1]

In some embodiments, R^{L} is —N(R′)C(S)N(R′)S(O)₂R^{s} wherein each variable is independently as described herein. In some embodiments, R^{L} is —NHC(S)N(R′)S(O)₂R^{s} wherein each variable is independently as described herein. In some embodiments, R^{L} is —N(R′)C(S)NHS(O)₂R^{s} wherein each variable is independently as described herein. In some embodiments, R^{L} is —NHC(S)NHS(O)₂R^{s} wherein R^{s} is as described herein. In some embodiments, R^{s} is R′ as described herein. In some embodiments, R^{s} is -L″-R′ wherein each of L″ and R′ is independently as described herein. In some embodiments, R^{s} is R′ as described herein. For example, in some embodiments, R^{s} is optionally substituted 5-6 membered heteroaryl having one heteroatom independently selected from nitrogen, oxygen and sulfur. In some embodiments, R^{s} is optionally substituted thienyl. In some embodiments, R^{s} is thienyl. In some embodiments, R^{s} is optionally substituted phenyl. In some embodiments, R^{s} is phenyl. In some embodiments, R^{s} is 4-(1, 1-dimethyl-2-fluoroethyl)phenyl. In some embodiments, R^{s} is 2, 5-difluorophenyl. In some embodiments, R^{s} is 3,5-difluorophenyl.

In some embodiments, R^{L} is —N(R′)C(NR′)N(R′)S(O)₂R^{s} wherein each variable is independently as described herein. In some embodiments, R^{L} is —NHC(NR′)N(R′)S(O)₂R^{s} wherein each variable is independently as described herein. In some embodiments, R^{L} is —N(R′)C(NH)N(R′)S(O)₂R^{s} wherein each variable is independently as described herein. In some embodiments, R^{L} is —N(R′)C(NR′)NHS(O)₂R^{s} wherein each variable is independently as described herein. In some embodiments, R^{L} is —N(R′)C(NH)NHS(O)₂R^{s} wherein each variable is independently as described herein. In some embodiments, R^{L} is —NHC(NR′)NHS(O)₂R^{s} wherein each variable is independently as described herein. In some embodiments, R^{L} is —NHC(NH)N(R′)S(O)₂R^{s} wherein each variable is independently as described herein. In some embodiments, R^{L} is —NHC(NH)NHS(O)₂R^{s} wherein R^{s} is as described herein. In some embodiments, R^{s} is R′ as described herein. In some embodiments, R^{s} is -L″-R′ wherein each of L″ and R′ is independently as described herein. In some embodiments, R^{s} is R′ as described herein. For example, in some embodiments, R^{s} is optionally substituted 5-6 membered heteroaryl having one heteroatom independently selected from nitrogen, oxygen and sulfur. In some embodiments, R^{s} is optionally substituted thienyl. In some embodiments, R^{s} is thienyl. In some embodiments, R^{s} is optionally substituted phenyl. In some embodiments, R^{s} is phenyl. In some embodiments, R^{s} is 4-(1, 1-dimethyl-2-fluoroethyl)phenyl. In some embodiments, R^{s} is 2, 5-difluorophenyl. In some embodiments, R^{s} is 3,5-difluorophenyl.

In some embodiments, R^{L} is —N(R′)C(O)C(O)N(R′)S(O)₂R^{s}, wherein each of R′ and R^{s} is independently as described herein. In some embodiments, R^{L} is —NHC(O)C(O)N(R′)S(O)₂R^{s}, wherein each of R′ and R^{s} is independently as described herein. In some embodiments, R^{L} is —N(R′)C(O)C(O)NHS(O)₂R^{s}, wherein each of R′ and R^{s} is independently as described herein. In some embodiments, R^{L} is —NHC(O)C(O)NHS(O)₂R^{s}, wherein R^{s} is as described herein. In some embodiments, R^{s} is -L″-R′ wherein each of L″ and R′ is independently as described herein. In some embodiments, R^{s} is R′ as described herein. For example, in some embodiments, R^{s} is optionally substituted 5-6 membered heteroaryl having one heteroatom independently selected from nitrogen, oxygen and sulfur. In some embodiments, R^{s} is optionally substituted thienyl. In some embodiments, R^{s} is thienyl. In some embodiments, R^{s} is optionally substituted phenyl. In some embodiments, R^{s} is phenyl. In some embodiments, R^{s} is 4-(1, 1-dimethyl-2-fluoroethyl)phenyl. In some embodiments, R^{s} is 2, 5-difluorophenyl. In some embodiments, R^{s} is 3,5-difluorophenyl.

In some embodiments, R^{L} is —N(R¹⁵)C(O)C(O)N(R¹⁶)S(O)₂R^{s}, wherein each of R¹⁵ and R¹⁶ is independently R′ as described herein, and R^{s} is independently as described herein. In some embodiments, R^{L} is —N(R′)C(O)N(R′)S(O)₂N(R^{s})₂ wherein each of R′ and R^{s} is independently as described herein. In some embodiments, R^{L} is —NHC(O)N(R′)S(O)₂N(R^{s})₂ wherein each of R′ and R^{s} is independently as described herein. In some embodiments, R^{L} is —N(R′)C(O)NHS(O)₂N(R^{s})₂ wherein each of R′ and R^{s} is independently as described herein. In some embodiments, R^{L} is —NHC(O)NHS(O)₂N(R^{s})₂ wherein each of R′ and R^{s} is independently as described herein. In some embodiments, R^{L} is —N(R′)C(O)N(R′)S(O)₂N(R′)₂ wherein each R′ is independently as described herein. In some embodiments, R^{L} is —NHC(O)NHS(O)₂N(R′)₂ wherein each R′ is independently as described herein. In some embodiments, R^{L} is —NHC(O)NHS(O)₂NHR′ wherein R′ is as described herein.

In some embodiments, R^{L} is —N(R′)S(O)₂R^{s} wherein each of R′ and R^{s} is independently as described herein. In some embodiments, R^{L} is —NHS(O)₂R^{s} wherein R^{s} is as described herein. In some embodiments, R^{L} is —NHS(O)₂R′ wherein R′ is as described herein. In some embodiments, R^{L} is —N(R′)S(O)₂R′ wherein each R′ is independently as described herein.

In some embodiments, R^{L} is —OC(O)N(R^{s})₂ wherein each R^{s} is independently as described herein. In some embodiments, R^{L} is —OC(O)N(R′)₂ wherein each R′ is independently as described herein. In some embodiments, R^{L} is —OC(O)NHR′ wherein R′ is as described herein.

In some embodiments, R^{L} is —OC(O)N(R′)C(O)N(R^{s})₂ wherein each R′ and R^{s} is independently as described herein. In some embodiments, R^{L} is —OC(O)N(R′)C(O)N(R′)₂ wherein each R′ is independently as described herein. In some embodiments, R^{L} is —OC(O)NHC(O)N(R′)₂ wherein each R′ is independently as described herein. In some embodiments, R^{L} is —OC(O)N(R′)C(O)NHR′ wherein R′ is as described herein. In some embodiments, R^{L} is —OC(O)NHC(O)N(R^{s})₂ wherein each R^{s} is independently as described herein.

In some embodiments, R^{L} is —OC(O)N(R′)C(O)R^{s} wherein each R′ and R^{s} is independently as described herein. In some embodiments, R^{L} is —OC(O)NHC(O)R^{s} wherein R^{s} is as described herein. In some embodiments, R^{L} is —OC(O)N(R′)C(O)R′ wherein each R′ is independently as described herein. In some embodiments, R^{L} is —OC(O)NHC(O)R′ wherein R′ is as described herein.

In some embodiments, R^{L} is —OC(O)N(R′)C(O)N(R′)S(O)₂R^{s} wherein each of R′ and R^{s} is independently as described herein. In some embodiments, R^{L} is —OC(O)NHC(O)N(R′)S(O)₂R^{s} wherein each of R′ and R^{s} is independently as described herein. In some embodiments, R^{L} is —OC(O)N(R′)C(O)NHS(O)₂R^{s} wherein each of R′ and R^{s} is independently as described herein. In some embodiments, R^{L} is —OC(O)NHC(O)NHS(O)₂R^{s} wherein R^{s} is as described herein. In some embodiments, R^{L} is —OC(O)N(R′)C(O)N(R′)S(O)₂R′ wherein each R′ is independently as described herein. In some embodiments, R^{L} is —OC(O)NHC(O)NHS(O)₂R′ wherein R′ is as described herein.

In some embodiments, R^{L} is —OC(O)N(R′)S(O)₂R^{s} wherein each of R′ and R^{s} is independently as described herein. In some embodiments, R^{L} is —OC(O)NHS(O)₂R^{s} wherein R^{s} is as described herein. In some embodiments, R^{L} is —OC(O)N(R′)S(O)₂R′ wherein each R′ is independently as described herein. In some embodiments, R^{L} is —OC(O)NHS(O)₂R′ wherein R′ is as described herein. In some embodiments, R′ is optionally substituted phenyl. In some embodiments, R′ is optionally substituted arylaliphatic as described herein. In some embodiments, R′ is optionally substituted arylheteroaliphatic as described herein. In some embodiments, R′ is optionally substituted heteroarylaliphatic as described herein. In some embodiments, R′ is optionally substituted heteroaryl-heteroaliphatic as described herein. In some embodiments, R′ is optionally substituted aliphatic-aryl as described herein. In some embodiments, R′ is optionally substituted heteroaliphatic-aryl as described herein. In some embodiments, R′ is optionally substituted aliphatic-heteroaryl as described herein. In some embodiments, R′ is optionally substituted heteroaliphatic-heteroaryl as described herein. In some embodiments, R^{s} is -L″-R′ as described herein. In some embodiments, L″ comprises -Cy- as described herein.

In some embodiments, the present disclosure provides a compound comprising two or more (e.g., 1-10, 1-5, 2, 3, 4, or 5) units, each unit is independently a compound of the present disclosure, e.g., a compound of formula I or a salt thereof. In some embodiments, a compound is has the structure of formula [A]-LD_[B] or a salt thereof, wherein each of A and B independently has such a structure that each of A-H and B—H is independently a compound as described herein, e.g., a compound of formula I or a salt thereof, and L^{D} is L as described herein. In some embodiments, in at least or each of A-H and B—H, R¹ and R^{1a} are —H. In some embodiments, L is optionally substituted C₁₋₁₀ alkylene wherein one or more —CH₂— are independently replaced with —O— or —N(R′)—. In some embodiments, R′ is —H or optionally substituted C₁₋₆ aliphatic. In some embodiments, R′ is —H. In some embodiments, R′ is —CH₃. In some embodiments, L is optionally substituted C₁₋₁₀ alkylene wherein one or more —CH₂— are independently replaced with —O— or —NH—. In some embodiments, one or more —CH₂— are replaced with —O—. In some embodiments, one or more —CH₂— are replaced with —N(R′)—. In some embodiments, one or more —CH₂— are replaced with —NH—. In some embodiments, two R^{L} are taken together to form L^{D} as described herein. In some embodiments two R^{s} of two R^{L} are taken together to form L^{D}. In some embodiments L^{D} is selected from:

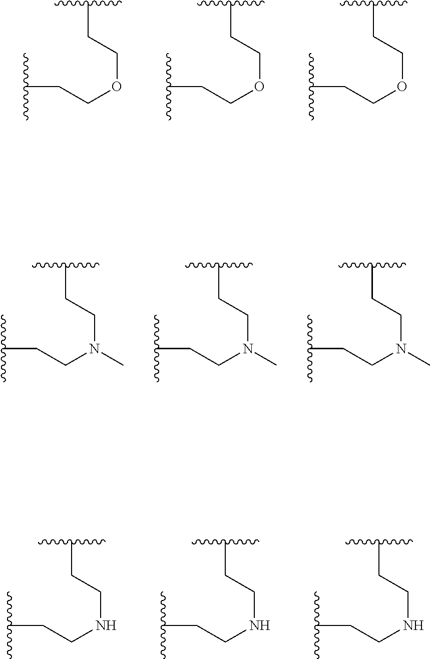

In some embodiments, R^{L} is R₁ as described herein. In some embodiments, R^{L} is as described in Table 1 to Table 7; those skilled in the art will appreciate that R^{L} or R¹ embodiments in these Tables may be utilized independently of m.

In some embodiments, -L¹-R^{L} is an optionally substituted C₁-C₁₂ aliphatic group. In some embodiments, -L¹-R^{L} is an optionally substituted C₁-C₁₂ alkyl group. In some embodiments, -L¹-R^{L} is an optionally substituted

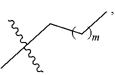

wherein m is as described herein. In some embodiments, -L¹-R^{L} is an optionally substituted

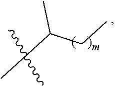

wherein m is as described herein. In some embodiments, -L-R^{L} is an optionally substituted

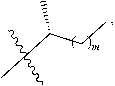

wherein m is as described herein. In some embodiments, -L¹-R^{L} is optionally substituted

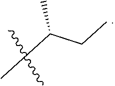 [SMILES: *[C@H](C)CC]

In some embodiments, -L¹-R^{L} is an optionally substituted

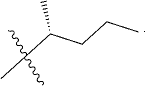 [SMILES: *[C@H](C)CCC]

In some embodiments, -L¹-R^{L} is an optionally substituted

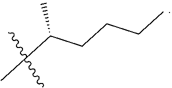 [SMILES: *[C@H](C)CCCC]

In some embodiments, -L¹-R^{L} is an optionally substituted

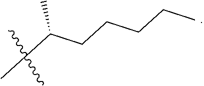 [SMILES: *[C@H](C)CCCCC]

In some embodiments, -L¹-R^{L} is an optionally substituted

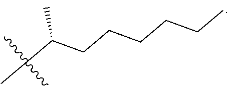 [SMILES: *[C@H](C)CCCCCC]

In some embodiments, -L¹-R^{L} is an optionally substituted

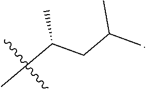 [SMILES: *[C@H](C)CC(C)C]

In some embodiments, -L¹-R^{L} is an optionally substituted

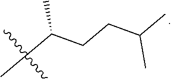 [SMILES: *[C@H](C)CCC(C)C]

In some embodiments, -L¹-R^{L} is an optionally substituted

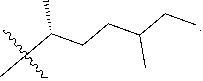 [SMILES: *[C@H](C)CCC(C)CC]

In some embodiments, -L¹-R^{L} is

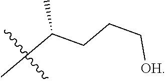 [SMILES: *[C@H](C)CCCO]

In some embodiments, -L¹-R^{L} is

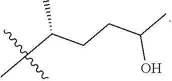 [SMILES: *[C@H](C)CCC(C)O]

In some embodiments, -L¹-R^{L} is

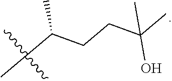 [SMILES: *[C@H](C)CCC(C)(C)O]

In some embodiments, -L¹-R^{L} is

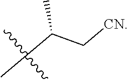 [SMILES: *[C@H](C)CC#N]

In some embodiments, -L¹-R^{L} is

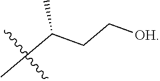 [SMILES: *[C@H](C)CCO]

In some embodiments, -L¹-R^{L} is

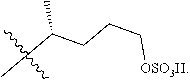

In some embodiments, -L¹-R^{L} is

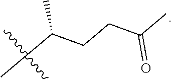 [SMILES: *[C@H](C)CCC(C)=O]

In some embodiments, -L¹-R^{L} is an optionally substituted C₂-C₁₂ alkenyl group. In some embodiments, -L¹-R^{L} is optionally substituted

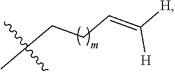

wherein m is as described herein. In some embodiments, -L¹-R^{L} is optionally substituted

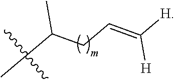

In some embodiments, -L¹-R^{L} is optionally substituted

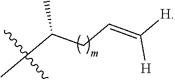

In some embodiments, -L¹-R^{L} is optionally substituted

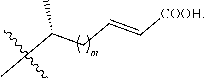

In some embodiments, -L¹-R^{L} is

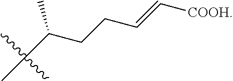 [SMILES: *[C@H](C)CC/C=C/C(=O)O]

In some embodiments, -L¹-R^{L} is optionally substituted

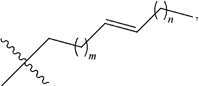

is as described herein and n is 0, 1, 2, or 3.

In some embodiments, -L¹-R^{L} is an optionally substituted C₂-C₁₂ alkynyl group.

In some embodiments, -L¹-R^{L} is optionally substituted

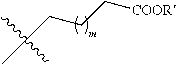

wherein m and R′ are as described herein. In some embodiments, -L¹-R^{L} is optionally substituted

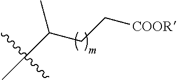

wherein m and R′ are as described herein. In some embodiments, -L¹-R^{L} is optionally substituted

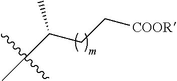

wherein m and R′ are as described herein. In some embodiments, m is 0. In some embodiments, m is 1. In some embodiments, m is 2. In some embodiments, m is 3. In some embodiments, R′ is H. In some embodiments, R′ is methyl. In some embodiments, R′ is ethyl. In some embodiments, R′ is isopropyl. In some embodiments, R′ is n-butyl. In some embodiments, R′ is t-butyl.

In some embodiments, -L¹-R^{L} is

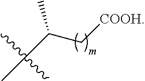

In some embodiments, -L¹-R^{L} is

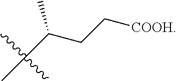 [SMILES: *[C@H](C)CCC(=O)O]

In some embodiments, -L¹-R^{L} is

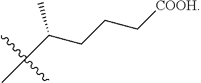 [SMILES: *[C@H](C)CCCC(=O)O]

In some embodiments, -L¹-R^{L} is

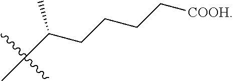 [SMILES: *[C@H](C)CCCCC(=O)O]

In some embodiments, -L¹-R^{L} is

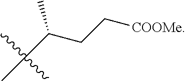 [SMILES: *[C@H](C)CCC(=O)OC]

In some embodiments, -L¹-R^{L} is optionally substituted

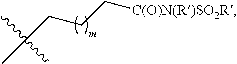

wherein m and each R′ is independently as described herein. In some embodiments, -L¹-R^{L} is optionally substituted

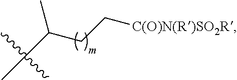

wherein m and each R′ is independently as described herein. In some embodiments, -L¹-R^{L} is optionally substituted

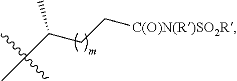

wherein m and each R′ is independently as described herein. In some embodiments, -L¹-R^{L} is optionally substituted

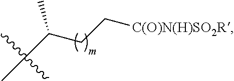

wherein m and R′ is independently as described herein. In some embodiments, m is 0. In some embodiments, m is 1. In some embodiments, m is 2. In some embodiments, m is 3. In some embodiments, R′ is H. In some embodiments, R′ is methyl. In some embodiments, R′ is ethyl. In some embodiments, R′ is isopropyl. In some embodiments, R′ is n-butyl. In some embodiments, R′ is t-butyl.

In some embodiments, -L¹-R^{L} is

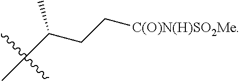

In some embodiments, -L¹-R^{L} is

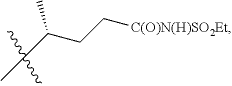

In some embodiments, -L¹-R^{L} is

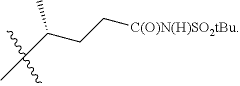

In some embodiments, -L¹-R^{L} is optionally substituted

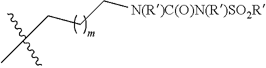

wherein m and each R′ are independently as described herein. In some embodiments, -L¹-R^{L} is optionally substituted

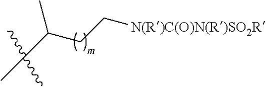

wherein m and each R′ are independently as described herein. In some embodiments, -L¹-R^{L} is optionally substituted

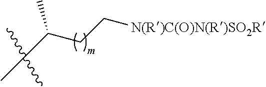

wherein m and each R′ are independently as described herein. In some embodiments, -L¹-R^{L} is optionally substituted

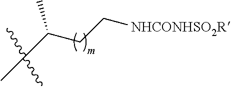

wherein m and R′ are independently as described herein. In some embodiments, m is 0. In some embodiments, m is 1. In some embodiments, m is 2. In some embodiments, m is 3.

In some embodiments, -L¹-R^{L} is

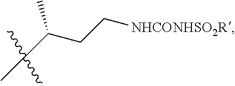

wherein R′ is selected from a group set forth below:

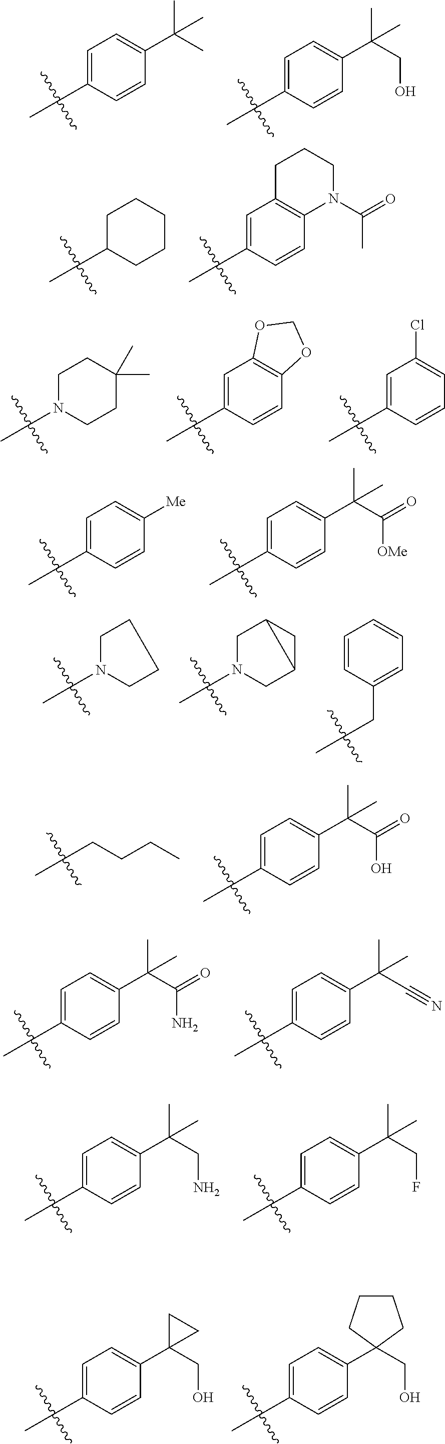

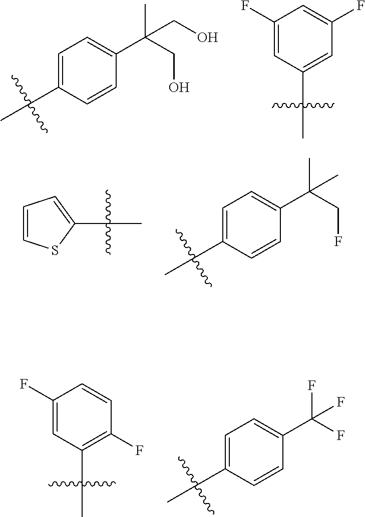 [SMILES: *c1ccc(C(C)(CO)CO)cc1 ; *c1cc(F)cc(F)c1 ; *c1cccs1 ; *c1ccc(C(C)(C)CF)cc1 ; *c1cc(F)ccc1F ; *c1ccc(C(F)(F)F)cc1]

In some embodiments, -L¹-R^{L} is optionally substituted

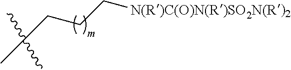

wherein m and each R′ are independently as described herein. In some embodiments, -L¹-R^{L} is optionally substituted

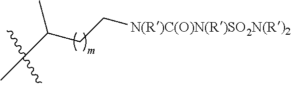

wherein m and each R′ are independently as described herein. In some embodiments, -L¹-R^{L} is optionally substituted

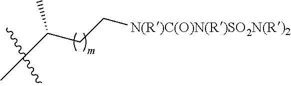

wherein m and each R′ are independently as described herein. In some embodiments, -L¹-R^{L} is optionally substituted

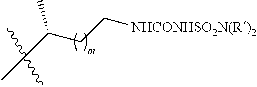

wherein m and R′ are independently as described herein. In some embodiments, m is 0. In some embodiments, m is 1. In some embodiments, m is 2. In some embodiments, m is 3.

In some embodiments, -L¹-R^{L} is

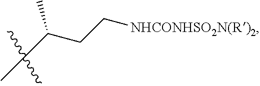

wherein each R′ is independently selected from H or optionally substituted C₁₋₁₀ aliphatic. In some embodiments, each R′ is independently selected from H or methyl. In some embodiments, wherein each R′ is independently selected from H or ethyl. In some embodiments, wherein each R′ is independently from H or isopropyl. In some embodiments, wherein each R′ is independently from H or n-butyl. In some embodiments, wherein each R′ is independently from H or t-butyl.

In some embodiments, -L¹-R^{L} is

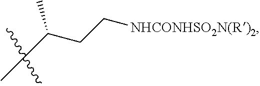

wherein two R′ groups are taken together with the nitrogen atom to form an optionally substituted, 3-15 membered, monocyclic, bicyclic or polycyclic ring having, in addition to the atom, 0-5 heteroatoms. In some embodiments, -L¹-R^{L} is

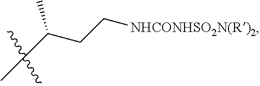

wherein two R′ groups are taken together with the nitrogen atom to form an optionally substituted, 3-10 membered, monocyclic, ring having, in addition to the atom, 1-3 heteroatoms. In some embodiments, -L¹-R^{L} is

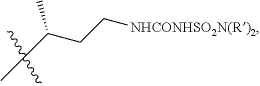

wherein two R′ groups are taken together with the nitrogen atom to form an optionally substituted group selected from

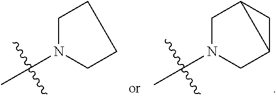 [SMILES: *N1CCCC1 ; *N1CC2CC2C1]

In some embodiments, R¹ and R^{1a} are —F, and -L¹-R^{L} is not —CH(CH₃)(CH₂)₂C(O)OR or a salt form thereof, wherein R is —H or optionally substituted C₁₋₆ aliphatic. In some embodiments, R¹ and R^{1a} are —F, and -L¹-R^{L} is not —(R)—CH(CH₃)(CH₂)₂C(O)OR or a salt form thereof, wherein R is —H or optionally substituted C₁₋₆ aliphatic. In some embodiments, R is —H. In some embodiments, R is —H or C₁₋₆ aliphatic. In some embodiments, R is —H or C₁₋₆ alkyl. In some embodiments, R¹ and R^{1a} are —F, and -L¹-R^{L} is not —CH(CH₃)(CH₂)₂C(O)OH or a salt form thereof. In some embodiments, R¹ and R^{1a} are —F, and -L¹-R^{L} is not —(R)—CH(CH₃)(CH₂)₂C(O)OH a salt form thereof.

In some embodiments, one of R¹ and one of R^{1a} is —F and the other is —H, and L¹ is or comprises —CH(CH₃)(CH₂)n-, wherein n is 1, 2 or 3, and —(CH₂)n- is bonded to —C(O)—, —O—, or a nitrogen atom. In some embodiments, R¹ and R^{1a} are —H, and L¹ is or comprises —CH(CH₃)(CH₂)n-, wherein n is 1, 2 or 3, and —(CH₂)n- is bonded to —C(O)—, —O—, or a nitrogen atom. In some embodiments, one of R¹ and one of R^{1a} is —F and the other is —H, and L¹ is or comprises —(R)—CH(CH₃)—(CH₂)n-, wherein n is 1, 2 or 3, and —(CH₂)n- is bonded to —C(O)—, —O—, or a nitrogen atom. In some embodiments, R¹ and R^{1a} are —H, and L¹ is or comprises —(R)—CH(CH₃)—(CH₂)n-, wherein n is 1, 2 or 3, and —(CH₂)n- is bonded to —C(O)—, —O—, or a nitrogen atom. As described herein, in some embodiments, n is 1. In some embodiments, n is 2. In some embodiments, n is 3. In some embodiments, —(CH₂)n- is bonded to —C(O)—. In some embodiments, —(CH₂)n- is bonded to —O—. In some embodiments, —(CH₂)n- is bonded to —OC(O)N(R′)—, wherein R′ is as described herein. In some embodiments, —(CH₂)n- is bonded to —OC(O)N(R′)S(O)₂—, wherein R′ is as described herein. In some embodiments, —(CH₂)n- is bonded to —OC(O)N(R′)S(O)₂R^{s}, wherein each variable is independently as described herein. In some embodiments, —(CH₂)n- is bonded to —OC(O)NHS(O)₂R^{s}, wherein R^{s} is as described herein. In some embodiments, —(CH₂)n- is bonded to —OC(O)NHS(O)₂R′, wherein R′ is as described herein. In some embodiments, —(CH₂)n- is bonded to —OC(O)NHS(O)₂R, wherein R is as described herein and is not —H. In some embodiments, R is or comprises an optionally substituted aryl or heteroaryl ring. In some embodiments, —(CH₂)n- is bonded to —N(R′)— wherein R′ is as described herein. In some embodiments, —(CH₂)n- is bonded to —NH—. In some embodiments, —(CH₂)n- is bonded to —N(R′)₂ wherein each R′ is independently as described herein. In some embodiments, —(CH₂)n- is bonded to —N(R′)C(O)—, wherein R′ is as described herein. In some embodiments, —(CH₂)n- is bonded to —N(R′)C(O)N(R′)—, wherein each R′ is independently as described herein. In some embodiments, —(CH₂)n- is bonded to —NHC(O)NH—. In some embodiments, —(CH₂)n- is bonded to —N(R′)C(O)N(R′)S(O)₂—, wherein each R′ is independently as described herein. In some embodiments, —(CH₂)n- is bonded to —NHC(O)NHS(O)₂—. In some embodiments, —(CH₂)n- is bonded to —N(R′)C(O)N(R′)S(O)₂R^{s}, wherein each variable is independently as described herein. In some embodiments, —(CH₂)n- is bonded to —NHC(O)NHS(O)₂R^{s}, wherein R^{s} is as described herein. In some embodiments, —(CH₂)n- is bonded to —NHC(O)NHS(O)₂R′, wherein R′ is as described herein. In some embodiments, —(CH₂)n- is bonded to —NHC(O)NHS(O)₂R, wherein R is as described herein and is not —H. In some embodiments, R is or comprises an optionally substituted aryl or heteroaryl ring. In some embodiments, —(CH₂)n- is bonded to —N(R′)C(O)C(O)N(R′)—, wherein each R′ is independently as described herein. In some embodiments, —(CH₂)n- is bonded to —NHC(O)C(O)NH—. In some embodiments, —(CH₂)n- is bonded to —N(R′)C(O)C(O)N(R′)S(O)₂—, wherein each R′ is independently as described herein. In some embodiments, —(CH₂)n- is bonded to —NHC(O)C(O)NHS(O)₂—. In some embodiments, —(CH₂)n- is bonded to —N(R′)C(O)C(O)N(R′)S(O)₂R^{s}, wherein each variable is independently as described herein. In some embodiments, —(CH₂)n- is bonded to —NHC(O)C(O)NHS(O)₂R^{s}, wherein R is as described herein. In some embodiments, —(CH₂)n- is bonded to —NHC(O)C(O)NHS(O)₂R′, wherein R′ is as described herein. In some embodiments, —(CH₂)n- is bonded to —NHC(O)C(O)NHS(O)₂R, wherein R is as described herein and is not-H. In some embodiments, R is or comprises an optionally substituted aryl or heteroaryl ring.

t

In some embodiments, t is 0. In some embodiments, t is 1-6. In some embodiments, t is 1. In some embodiments, t is 2. In some embodiments, t is 3. In some embodiments, t is 4. In some embodiments, t is 5. In some embodiments, t is 6.

### R^{t}

In some embodiments, R^{t} is R′ as described herein. In some embodiments, R^{t} is halogen. In some embodiments, R^{t} is —F. In some embodiments, R^{t} is —Cl. In some embodiments, R^{t} is —Br. In some embodiments, R^{t} is —I. In some embodiments, R^{t} is —CN. In some embodiments, R^{t} is —N₃. In some embodiments, R^{t} is —OR′ wherein R′ is as described herein. In some embodiments, R^{t} is —OR wherein R is as described herein. In some embodiments, R^{t} is —C(O)R′ wherein R′ is as described herein. In some embodiments, R^{t} is —S(O)₂R′ wherein R′ is as described herein. In some embodiments, R^{t} is —S(O)₂N(R′)₂ wherein each R′ is independently as described herein. In some embodiments, R^{t} is —SO₃R′ wherein R′ is as described herein. In some embodiments, R^{t} is —OS(O)₂R′ wherein R′ is as described herein. In some embodiments, R^{t} is —OP(O)(R′)₂ wherein each R′ is independently as described herein. In some embodiments, R^{t} is —OP(O)(OR′)₂ wherein each R′ is independently as described herein. In some embodiments, R^{t} is —P(O)(R′)₂ wherein each R′ is independently as described herein. In some embodiments, R^{t} is —PO(OR′)₂ wherein each R′ is independently as described herein. In some embodiments, R^{t} is —SR′ wherein R′ is as described herein. In some embodiments, R^{t} is —C(O)N(R′)₂ wherein each R′ is independently as described herein. In some embodiments, R^{t} is —N(R′)₂ wherein each R′ is independently as described herein. In some embodiments, R^{t} is —N(R)₂ wherein each R is independently as described herein.

### Ring A

As described herein, Ring A is optionally substituted (in addition to the group it is bonded to and the R^{t} groups). In some embodiments, Ring A is substituted. In some embodiments, Ring A is unsubstituted.

In some embodiments, Ring A is 3-20, 3-15, 3-10, 3-8, 3-6, 5-6, or 3, 4, 5, 6, 7, 8, 9, 10, 11, 12, 13, 14, 15, 16, 17, 18, 19, or 20, etc.) membered. In some embodiments, Ring A is 3-membered. In some embodiments, Ring A is 4-membered. In some embodiments, Ring A is 5-membered. In some embodiments, Ring A is 6-membered. In some embodiments, Ring A is 7-membered. In some embodiments, Ring A is 8-membered. In some embodiments, Ring A is 9-membered. In some embodiments, Ring A is 10-membered. In some embodiments, Ring A is 11-membered. In some embodiments, Ring A is 12-membered. In some embodiments, Ring A is saturated. In some embodiments, Ring A is partially unsaturated. In some embodiments, Ring A is aromatic. In some embodiments, Ring A is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, Ring A has 1-10 (e.g., 1, 2, 3, 4, 5, 6, 7, 8, 9, or 10, etc.) heteroatoms independently selected from oxygen, nitrogen, sulfur, phosphorus and silicon. In some embodiments, Ring A has 1-5 (e.g., 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, Ring A is an optionally substituted 5-10 membered aromatic ring having 0-5 heteroatoms independently selected from oxygen, nitrogen, and sulfur. In some embodiments, Ring A is an optionally substituted 5-6 membered aromatic ring having 0-5 heteroatoms independently selected from oxygen, nitrogen, and sulfur. In some embodiments, Ring A is an optionally substituted phenyl ring. In some embodiments, Ring A is a phenyl ring. In some embodiments, Ring A is an optionally substituted 10-membered bicyclic aryl ring. In some embodiments, Ring A is an optionally substituted 5-9 membered heteroaryl ring having 1-4 heteroatoms independently selected from nitrogen, oxygen and sulfur. In some embodiments, Ring A is an optionally substituted 5-membered heteroaryl ring having 1-4 heteroatoms independently selected from nitrogen, oxygen and sulfur. In some embodiments, Ring A is an optionally substituted 6-membered heteroaryl ring having 1-4 heteroatoms independently selected from nitrogen, oxygen and sulfur. In some embodiments, Ring A is an optionally substituted 9-membered bicyclic heteroaryl ring having 1-4 heteroatoms independently selected from nitrogen, oxygen and sulfur. In some embodiments, a heteroatom is nitrogen.

### L″

In some embodiments, L″ is a covalent bond. In some embodiments, L″ is an optionally substituted, bivalent C₁₋₆ (e.g., C₁₋₄, C₁, C₂, C₃ or C₄) aliphatic or heteroaliphatic group having 1-10 (e.g., 1, 2, 3, 4, 5, 6, 7, 8, 9, or 10) heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, L″ is an optionally substituted, bivalent C₁₋₆ aliphatic wherein one or more methylene units of the group are optionally and independently replaced as described herein. In some embodiments, L″ is an optionally substituted, bivalent C₁₋₄ aliphatic wherein one or more methylene units of the group are optionally and independently replaced as described herein. In some embodiments, L″ is an optionally substituted, bivalent C₁₋₆ heteroaliphatic group having 1-10 heteroatoms wherein one or more methylene units of the group are optionally and independently replaced as described herein. In some embodiments, L″ is an optionally substituted, bivalent C₁₋₄ heteroaliphatic group having 1-10 heteroatoms wherein one or more methylene units of the group are optionally and independently replaced as described herein. In some embodiments, L″ is an optionally substituted, bivalent C₁₋₄ alkylene wherein one or more methylene units of the group are optionally and independently replaced as described herein. In some embodiments, at least one methylene unit is replaced as described herein. In some embodiments, at least one methylene unit is replaced with -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—. In some embodiments, a methylene unit is replaced with -Cy-. In some embodiments, it is replaced with —O—. In some embodiments, it is replaced with —S—. In some embodiments, it is replaced with —S—S—. In some embodiments, it is replaced with —N(R′)—. In some embodiments, it is replaced with —C(O)—. In some embodiments, it is replaced with —C(O)S—. In some embodiments, it is replaced with —C(O)O—. In some embodiments, it is replaced with —C(S)—. In some embodiments, it is replaced with —C(NR′)—. In some embodiments, it is replaced with —C(O)N(R′)—. In some embodiments, it is replaced with —C(NR′)N(R′)—. In some embodiments, it is replaced with —C(S)N(R′)—. In some embodiments, it is replaced with —N(R′)C(O)N(R′)—. In some embodiments, it is replaced with —N(R′)C(NR′)N(R′)—. In some embodiments, it is replaced with —N(R′)C(S)N(R′)—. In some embodiments, it is replaced with —N(R′)C(O)O—. In some embodiments, it is replaced with —N(R′)C(O)N(R′)S(O)₂—. In some embodiments, it is replaced with —OC(O)N(R′)—. In some embodiments, it is replaced with —OC(O)N(R′)S(O)₂—. In some embodiments, it is replaced with —S(O)—. In some embodiments, it is replaced with —S(O)₂—. In some embodiments, it is replaced with —S(O)₂N(R′)—. In some embodiments, it is replaced with —P(O)(OR′)—. In some embodiments, it is replaced with —P(O)(OR′)O—.

In some embodiments, L″ is optionally substituted bivalent C₁₋₆ aliphatic. In some embodiments, L″ is optionally substituted bivalent C₁₋₄ aliphatic. In some embodiments, L″ is optionally substituted bivalent C₁₋₆ alkylene. In some embodiments, L″ is optionally substituted bivalent C₁₋₄ alkylene. In some embodiments, L″ is optionally substituted —CH₂—. In some embodiments, L″ is optionally substituted —(CH₂)₂—. In some embodiments, L″ is optionally substituted —(CH₂)₃—. In some embodiments, L″ is optionally substituted —(CH₂)₄—.

s

As described herein, in some embodiments, in formula I there are one or more R^{s} groups each of which is independently as described herein. In some embodiments, s is 0. In some embodiments, s is 1-25. In some embodiments, s is 2-25. In some embodiments, s is 1-20. In some embodiments, s is 1-15. In some embodiments, s is 1-10. In some embodiments, s is 1-5. In some embodiments, s is 1, 2, 3, 4, 5, 6, 7, 8, 9, 10, 11, 12, 13, 14, 15, 16, 17, 18, 19, 20, 21, 22, 23, 24, or 25. In some embodiments, s is 1. In some embodiments, s is 2. In some embodiments, s is 3. In some embodiments, s is 4. In some embodiments, s is 5. In some embodiments, s is 6. In some embodiments, s is 7. In some embodiments, s is 8. In some embodiments, s is 9. In some embodiments, s is 10. In some embodiments, s is 10-15. In some embodiments, s is 16-20. In some embodiments, s is 21-25.

### R^{x}

In some embodiments, R^{x} is -L-R′ wherein each of L and R′ is independently as described herein. In some embodiments, R^{x} is -L″-R′ wherein each of L″ and R′ is independently as described herein. In some embodiments, R^{x} is R′ as described herein. In some embodiments, R^{x} is R as described herein. In some embodiments, R is —H.

In some embodiments, R^{x} is —Si(R′)₃ wherein each R′ is independently as described herein. In some embodiments, none of R′ is —H. In some embodiments, each R′ is independently R as described herein and R is not —H. In some embodiments, each R is independently an optionally substituted group selected from C₁₋₆ aliphatic and phenyl. In some embodiments, each R is independently an optionally substituted group selected from C₁₋₆ alkyl and phenyl. In some embodiments, each R is independently an optionally substituted C₁₋₆ alkyl.

In some embodiments, R is a hydroxyl protecting group, which is widely known and can be utilized in accordance with the present disclosure.

### -Cy-

As described herein, -Cy- is optionally substituted (in addition to the two group it is bonded to). In some embodiments, -Cy- is substituted. In some embodiments, -Cy- is unsubstituted.

In some embodiments, -Cy- is 3-20, 3-15, 3-10, 3-8, 3-6, 5-6, or 3, 4, 5, 6, 7, 8, 9, 10, 11, 12, 13, 14, 15, 16, 17, 18, 19, or 20, etc.) membered. In some embodiments, -Cy- is 3-membered. In some embodiments, -Cy- is 4-membered. In some embodiments, -Cy- is 5-membered. In some embodiments, -Cy-is 6-membered. In some embodiments, -Cy- is 7-membered. In some embodiments, -Cy- is 8-membered. In some embodiments, -Cy- is 9-membered. In some embodiments, -Cy- is 10-membered. In some embodiments, -Cy- is 11-membered. In some embodiments, -Cy- is 12-membered. In some embodiments, -Cy- is saturated. In some embodiments, -Cy- is partially unsaturated. In some embodiments, -Cy- is aromatic. In some embodiments, -Cy- is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, -Cy- has 1-10 (e.g., 1, 2, 3, 4, 5, 6, 7, 8, 9, or 10, etc.) heteroatoms independently selected from oxygen, nitrogen, sulfur, phosphorus and silicon. In some embodiments, -Cy- has 1-5 (e.g., 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, -Cy- is an optionally substituted 5-10 membered aromatic ring having 0-5 heteroatoms independently selected from oxygen, nitrogen, and sulfur. In some embodiments, -Cy- is an optionally substituted 5-6 membered aromatic ring having 0-5 heteroatoms independently selected from oxygen, nitrogen, and sulfur. In some embodiments, -Cy- is an optionally substituted phenyl ring. In some embodiments, -Cy- is a phenyl ring. In some embodiments, -Cy- is an optionally substituted 10-membered bicyclic aryl ring. In some embodiments, -Cy- is an optionally substituted 5-9 membered heteroaryl ring having 1-4 heteroatoms independently selected from nitrogen, oxygen and sulfur. In some embodiments, -Cy- is an optionally substituted 5-membered heteroaryl ring having 1-4 heteroatoms independently selected from nitrogen, oxygen and sulfur. In some embodiments, -Cy- is an optionally substituted 6-membered heteroaryl ring having 1-4 heteroatoms independently selected from nitrogen, oxygen and sulfur. In some embodiments, -Cy- is an optionally substituted 9-membered bicyclic heteroaryl ring having 1-4 heteroatoms independently selected from nitrogen, oxygen and sulfur. In some embodiments, a heteroatom is nitrogen.

### R′

In some embodiments, R′ is R as described herein. In some embodiments, R′ is —H. In some embodiments, R′ is —OR wherein R is as described herein. In some embodiments, R′ is —C(O)R wherein R is as described herein. In some embodiments, R′ is —C(O)R wherein R is as described herein. In some embodiments, R′ is —C(O)OR wherein R is as described herein. In some embodiments, R′ is —C(O)N(R)₂, wherein each R is independently as described herein. In some embodiments, R′ is —S(O)₂R wherein R is as described herein. In some embodiments, R′ is —S(O)₂CH₃. In some embodiments, R′ is —C(O)CH₃. In some embodiments, R′ is —C(O)R wherein R is optionally substituted C₆₋₁₄ aryl. In some embodiments, R′ is —C(O)R wherein R is optionally substituted phenyl. In some embodiments, R′ is —C(O)R wherein R is 3-iodophenyl. In some embodiments, R′ is —C(O)OR wherein R is as described herein. In some embodiments, R′ is —C(O)OH. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl.

In some embodiments, R′ is a suitable group described in Table 1 to Table 7.

### R

Various embodiments for R are extensively described herein, including in various sections for other variables that can be R (e.g., R^{s}, R^{L}, R′, etc.).

In some embodiments, R is —H. In some embodiments, R is not —H.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₁₄ aryl-C₁₋₁₀ aliphatic, C₆₋₁₄ aryl-C₁₋₁₀ heteroaliphatic having 1-5 heteroatoms, C₁₋₁₀ aliphatic-C₆₋₁₄ aryl, C₁₋₁₀ heteroaliphatic having 1-5 heteroatoms-C₆₋₁₄ aryl, 5-14 membered heteroaryl having 1-5 heteroatoms, 5-14 membered heteroaryl having 1-5 heteroatoms-C₁₋₁₀ aliphatic, 5-14 membered heteroaryl having 1-5 heteroatoms-C₁₋₁₀ heteroaliphatic having 1-5 heteroatoms, C₁₋₁₀ aliphatic-5-14 membered heteroaryl having 1-5 heteroatoms, C₁₋₁₀ heteroaliphatic having 1-5 heteroatoms-5-14 membered heteroaryl having 1-5 heteroatoms, C₂₋₂₀ biaryl having 0-10 heteroatoms, and 3-15 membered heterocyclyl having 1-5 heteroatoms.

In some embodiments, R is optionally substituted C₁₋₁₅ (e.g., C₁₋₁₅, C₁₋₁₂, C₁₋₁₀, etc.) aliphatic. In some embodiments, R is optionally substituted C₁₋₁₀ aliphatic. In some embodiments, an aliphatic group is an alkyl group. In some embodiments, R is C₁₋₆ aliphatic. In some embodiments, R is C₁₋₆ alkyl. In some embodiments, R is optionally substituted methyl. In some embodiments, R is optionally substituted ethyl. In some embodiments, R is optionally substituted n-propyl. In some embodiments, R is optionally substituted isopropyl. In some embodiments, R is n-butyl. In some embodiments, R is t-butyl. In some embodiments, R is pentyl. In some embodiments, R is hexyl.

In some embodiments, an aliphatic group is or comprises a cycloaliphatic ring. In some embodiments, R is optionally substituted C₃₋₁₅ (e.g., C₃₋₁₅, C₃₋₁₂, C₃₋₁₀, C₄₋₁₀, C₃₋₉, C₃₋₇, or 3, 4, 5, 6, 7, 8, 9, 10, 11, 12, 13, 14, or 15 etc.) cycloaliphatic. In some embodiments, R is optionally substituted C₃₋₁₀ cycloaliphatic. In some embodiments, an aliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl.

In some embodiments, R is optionally substituted C₁₋₁₅ (e.g., C₁₋₁₅, C₁₋₁₂, C₁₋₁₀, etc.) heteroaliphatic having 1-5 (e.g., 1, 2, 3, 4, or 5) heteroatoms independently selected from oxygen, nitrogen, sulfur, phosphorus and silicon. In some embodiments, R is optionally substituted C₁₋₁₅ (e.g., C₁₋₁₅, C₁₋₁₂, C₁₋₁₀, etc.) heteroaliphatic having 1-5 (e.g., 1, 2, 3, 4, or 5) heteroatoms independently selected from nitrogen, oxygen and sulfur. In some embodiments, R is C₁₋₁₀ heteroaliphatic having 1-5 (e.g., 1, 2, 3, 4, or 5, etc.) heteroatoms independently selected from nitrogen, oxygen and sulfur. In some embodiments, R is C₁₋₁₀ heteroaliphatic having 1-2 (e.g., 1 or 2) heteroatoms independently selected from nitrogen, oxygen and sulfur. In some embodiments, R is C₁₋₁₀ heteroaliphatic having one heteroatom selected from nitrogen, oxygen and sulfur. In some embodiments, a heteroatom is nitrogen. In some embodiments, a heteroatom is oxygen. In some embodiments, a heteroatom is sulfur.

In some embodiments, R is optionally substituted C₆₋₁₄ (e.g., C₆₋₁₄, C₆₋₁₀, C₆₋₉, etc.) aryl. In some embodiments, R is optionally substituted C₆₋₁₀ aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted C₆₋₁₄ aryl-C₁₋₁₅ aliphatic, wherein the aryl and aliphatic are independently as described herein. In some embodiments, R is optionally substituted C₆₋₁₀ aryl-C₁₋₁₅ aliphatic. In some embodiments, R is optionally substituted C₆₋₁₀ aryl-C₁₋₁₀ aliphatic. In some embodiments, R is optionally substituted C₆ aryl-C₁₋₁₀ aliphatic. In some embodiments, R is optionally substituted C₆₋₁₀ aryl-C₁₋₁₀ alkyl. In some embodiments, R is optionally substituted phenyl-C₁₋₁₅ aliphatic. Various suitable aryl and aliphatic groups are as described herein.

In some embodiments, R is optionally substituted C₆₋₁₄ aryl-C₁₋₁₅ heteroaliphatic having 1-5 heteroatoms wherein the aryl and heteroaliphatic are independently as described herein. In some embodiments, R is optionally substituted C₆₋₁₀ aryl-C₁₋₁₅ heteroaliphatic having 1-5 heteroatoms. In some embodiments, R is optionally substituted C₆₋₁₀ aryl-C₁₋₁₀ heteroaliphatic having 1-5 heteroatoms. Various suitable aryl and heteroaliphatic groups are as described herein.

In some embodiments, R is optionally substituted C₁₋₁₅ aliphatic-C₆₋₁₄ aryl wherein the aliphatic and aryl groups are independently as described herein. In some embodiments, R is optionally substituted C₁₋₁₀ aliphatic-C₆₋₁₄ aryl. In some embodiments, R is optionally substituted C₁₋₁₀ aliphatic-C₆₋₁₀ aryl. In some embodiments, R is optionally substituted C₁₋₁₅ aliphatic-C₆₋₁₀ aryl. Various suitable aryl and aliphatic groups are as described herein.

In some embodiments, R is optionally substituted C₁₋₁₅ heteroaliphatic having 1-5 heteroatoms-C₆₋₁₄ aryl wherein the heteroaliphatic and aryl are independently as described herein. In some embodiments, R is optionally substituted C₁₋₁₀ heteroaliphatic having 1-5 heteroatoms-C₆₋₁₄ aryl. In some embodiments, R is optionally substituted C₁₋₁₅ heteroaliphatic having 1-5 heteroatoms-C₆₋₁₀ aryl. In some embodiments, R is optionally substituted C₁₋₁₀ heteroaliphatic having 1-5 heteroatoms-C₆₋₁₀ aryl. Various suitable aryl and heteroaliphatic groups are as described herein.

In some embodiments, R is optionally substituted 5-14 (e.g., 5-10, 5-9, or 5, 6, 7, 8, 9, 10, 11, 12, 13, 14, or 15 etc.) membered heteroaryl having 1-10 (e.g., 1-9, 1-8, 1-6, 1-5, 1-4, or 1, 2, 3, 4, 5, 6, 7, 8, 9, or 10 etc.) heteroatoms independently selected from oxygen, nitrogen, sulfur, phosphorus and silicon. In some embodiments, R is 5-14 (e.g., 5-10, 5-9, or 5, 6, 7, 8, 9, 10, 11, 12, 13, 14, or 15 etc.) membered heteroaryl having 1-10 (e.g., 1-9, 1-8, 1-6, 1-5, 1-4, or 1, 2, 3, 4, 5, 6, 7, 8, 9, or 10 etc.) heteroatoms independently selected from oxygen, nitrogen, sulfur, phosphorus and silicon. In some embodiments, R is optionally substituted 5-14 (e.g., 5-10, 5-9, or 5, 6, 7, 8, 9, 10, 11, 12, 13, 14, or 15 etc.) membered heteroaryl having 1-5 (e.g., 1-5, 1-4, or 1, 2, 3, 4, or 5 etc.) heteroatoms independently selected from oxygen, nitrogen, sulfur, phosphorus and silicon. In some embodiments, R is 5-14 (e.g., 5-10, 5-9, or 5, 6, 7, 8, 9, 10, 11, 12, 13, 14, or 15 etc.) membered heteroaryl having 1-5 (e.g., 1-5, 1-4, or 1, 2, 3, 4, or 5 etc.) heteroatoms independently selected from oxygen, nitrogen, sulfur, phosphorus and silicon. In some embodiments, R is optionally substituted 5-10 (e.g., 5-9, or 5, 6, 9, 10 etc.) membered heteroaryl having 1-4 (e.g., 1, 2, 3, or 4, etc.) heteroatoms independently selected from nitrogen, oxygen and sulfur. In some embodiments, R is 5-10 (e.g., 5-9, or 5, 6, 9, 10 etc.) membered heteroaryl having 1-4 (e.g., 1, 2, 3, or 4, etc.) heteroatoms independently selected from nitrogen, oxygen and sulfur. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from nitrogen, oxygen and sulfur, wherein at least one monocyclic unit contains 1-4 heteroatoms. In some embodiments, R is optionally substituted 5-membered monocyclic heteroaryl having 1-4 heteroatoms independently selected from nitrogen, oxygen and sulfur. In some embodiments, R is optionally substituted 6-membered monocyclic heteroaryl having 1-4 heteroatoms independently selected from nitrogen, oxygen and sulfur. In some embodiments, R is optionally substituted 9-membered bicyclic heteroaryl having 1-4 heteroatoms independently selected from nitrogen, oxygen and sulfur. In some embodiments, R is optionally substituted 10-membered bicyclic heteroaryl having 1-4 heteroatoms independently selected from nitrogen, oxygen and sulfur. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur. In some embodiments, each heteroatom is independently selected from nitrogen, oxygen and sulfur.

In some embodiments, R is optionally substituted 5-14 membered heteroaryl having 1-5 heteroatoms-C₁₋₁₅ aliphatic wherein the heteroaryl and aliphatic are independently as described herein. In some embodiments, R is optionally substituted 5-14 membered heteroaryl having 1-5 heteroatoms-C₁₋₁₅ aliphatic. In some embodiments, R is optionally substituted 5-10 membered heteroaryl having 1-5 heteroatoms-C₁₋₁₅ aliphatic. In some embodiments, R is optionally substituted 5-10 membered heteroaryl having 1-5 heteroatoms-C₁₋₁₀ aliphatic. Various suitable heteroaryl and aliphatic groups are as described herein.

In some embodiments, R is optionally substituted 5-14 membered heteroaryl having 1-10 heteroatoms-C₁₋₁₅ heteroaliphatic having 1-5 heteroatoms wherein the heteroaryl and heteroaliphatic are independently as described herein. In some embodiments, R is optionally substituted 5-14 membered heteroaryl having 1-5 heteroatoms-C₁₋₁₅ heteroaliphatic having 1-5 heteroatoms. In some embodiments, R is optionally substituted 5-10 membered heteroaryl having 1-5 heteroatoms-C₁₋₁₅ heteroaliphatic having 1-5 heteroatoms. In some embodiments, R is optionally substituted 5-14 membered heteroaryl having 1-10 heteroatoms-C₁₋₁₀ heteroaliphatic having 1-5 heteroatoms. In some embodiments, R is optionally substituted 5-14 membered heteroaryl having 1-5 heteroatoms-C₁₋₁₀ heteroaliphatic having 1-5 heteroatoms. In some embodiments, R is optionally substituted 5-10 membered heteroaryl having 1-5 heteroatoms-C₁₋₁₀ heteroaliphatic having 1-5 heteroatoms. Various suitable heteroaryl and heteroaliphatic groups are as described herein.

In some embodiments, R is optionally substituted C₁₋₁₅ aliphatic-5-14 membered heteroaryl having 1-10 heteroatoms wherein the aliphatic and heteroaryl are independently as described herein. In some embodiments, R is optionally substituted C₁₋₁₅ aliphatic-5-14 membered heteroaryl having 1-5 heteroatoms. In some embodiments, R is optionally substituted C₁₋₁₀ aliphatic-5-14 membered heteroaryl having 1-10 heteroatoms. In some embodiments, R is optionally substituted C₁₋₁₀ aliphatic-5-14 membered heteroaryl having 1-5 heteroatoms. In some embodiments, R is optionally substituted C₁₋₁₅ aliphatic-5-10 membered heteroaryl having 1-5 heteroatoms. In some embodiments, R is optionally substituted C₁₋₁₀ aliphatic-5-10 membered heteroaryl having 1-5 heteroatoms. Various suitable heteroaryl and aliphatic groups are as described herein.

In some embodiments, R is optionally substituted C₁₋₁₅ heteroaliphatic having 1-5 heteroatoms-5-14 membered heteroaryl having 1-10 heteroatoms wherein the heteroaliphatic and heteroaryl are independently as described herein. In some embodiments, R is optionally substituted C₁₋₁₅ heteroaliphatic having 1-5 heteroatoms-5-14 membered heteroaryl having 1-5 heteroatoms. In some embodiments, R is optionally substituted C₁₋₁₅ heteroaliphatic having 1-5 heteroatoms-5-10 membered heteroaryl having 1-5 heteroatoms. In some embodiments, R is optionally substituted C₁₋₁₀ heteroaliphatic having 1-5 heteroatoms-5-14 membered heteroaryl having 1-10 heteroatoms. In some embodiments, R is optionally substituted C₁₋₁₀ heteroaliphatic having 1-5 heteroatoms-5-14 membered heteroaryl having 1-5 heteroatoms. In some embodiments, R is optionally substituted C₁₋₁₀ heteroaliphatic having 1-5 heteroatoms-5-10 membered heteroaryl having 1-5 heteroatoms. Various suitable heteroaryl and heteroaliphatic groups are as described herein.

In some embodiments, R is optionally substituted C₂-C₂₀ (e.g., C₂₋₁₅, C₂₋₁₈, C₂₋₁₅, C₂₋₁₂, C₂₋₁₁₀, C₆₋₂₀, C₈₋₂₀, C₈₋₁₂, etc.) biaryl having 0-10 (e.g., 1-10, 2-10, 2-8, 2-6, 0, 1, 2, 3, 4, 5, 6, 7, 8, 9, or 10 etc.) heteroatoms. In some embodiments, each aromatic unit is independently 5-10 (e.g., 5, 6, 9, or 10, etc.) membered and independently has 0-10 (e.g., 1-10, 2-10, 2-8, 2-6, 0, 1, 2, 3, 4, 5, 6, 7, 8, 9, or 10 etc.) heteroatoms. In some embodiments, an aromatic unit is an aryl group as described herein. In some embodiments, an aromatic unit is phenyl. In some embodiments, an aromatic unit is a heteroaryl group as described herein. In some embodiments, R is optionally substituted biphenyl. In some embodiments, an aromatic unit is an aryl group as described herein, and an aromatic unit is a heteroaryl group as described herein. In some embodiments, both aromatic unit are independently a heteroaryl as described herein. In some embodiments, an aromatic unit is 5-membered heteroaryl having 1-5 heteroatoms as described herein. In some embodiments, an aromatic unit is 6-membered heteroaryl having 1-5 heteroatoms as described herein. In some embodiments, an aromatic unit is 9-membered bicyclic heteroaryl having 1-5 heteroatoms as described herein. In some embodiments, an aromatic unit is 10-membered bicyclic heteroaryl having 1-5 heteroatoms as described herein. In some embodiments, an aromatic unit is phenyl. In some embodiments, an aromatic unit is naphthyl. In some embodiments, each aromatic unit is independently selected from 5-membered heteroaryl having 1-5 heteroatoms, 6-membered heteroaryl having 1-5 heteroatoms, 9-membered bicyclic heteroaryl having 1-5 heteroatoms, 10-membered bicyclic heteroaryl having 1-5 heteroatoms, phenyl and naphthyl. In some embodiments, each aromatic unit is independently and optionally substituted. In some embodiments, R is optionally substituted biaryl having 1-10 heteroatoms. In some embodiments, a biaryl group has 1 heteroatom. In some embodiments, a biaryl group has 2 heteroatoms. In some embodiments, a biaryl group has 3 heteroatoms. In some embodiments, a biaryl group has 4 heteroatoms. In some embodiments, a biaryl group has 5 heteroatoms. In some embodiments, a biaryl group has 6 heteroatoms. In some embodiments, a heteroatom is nitrogen. In some embodiments, a heteroatom is oxygen. In some embodiments, a heteroatom is sulfur.

In some embodiments, R is optionally substituted 3-20 (e.g., 3-15, 3-10, or 3, 4, 5, 6, 7, 8, 9, 10, 11, 12, 13, 14, 15, 16, 17, 18, 19, or 20, etc.) membered heterocyclyl having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is optionally substituted 3-20 (e.g., 3-15, 3-10, or 3, 4, 5, 6, 7, 8, 9, 10, 11, 12, 13, 14, 15, 16, 17, 18, 19, or 20, etc.) membered heterocyclyl having 1-10 (e.g., 1, 2, 3, 4, 5, 6, 7, 8, 9, or 10, etc.) heteroatoms independently selected from nitrogen, oxygen and sulfur. In some embodiments, R is optionally substituted 3-20 (e.g., 3-20, 3-15, 3-10, or 3, 4, 5, 6, 7, 8, 9, 10, 11, 12, 13, 14, 15, 16, 17, 18, 19, or 20, etc.) membered heterocyclyl having 1-5 (e.g., 1, 2, 3, 4, or 5, etc.) heteroatoms independently selected from nitrogen, oxygen and sulfur. In some embodiments, R is optionally substituted 3-10 (e.g., 3, 4, 5, 6, 7, 8, 9, or 10, etc.) membered heterocyclyl having 1-5 (e.g., 1, 2, 3, 4, or 5, etc.) heteroatoms independently selected from nitrogen, oxygen and sulfur. In some embodiments, R is 3-20 (e.g., 3-15, 3-10, or 3, 4, 5, 6, 7, 8, 9, 10, 11, 12, 13, 14, 15, 16, 17, 18, 19, or 20, etc.) membered heterocyclyl having 1-10 (e.g., 1, 2, 3, 4, 5, 6, 7, 8, 9, or 10, etc.) heteroatoms independently selected from oxygen, nitrogen, sulfur, phosphorus and silicon. In some embodiments, R is 3-20 (e.g., 3-15, 3-10, or 3, 4, 5, 6, 7, 8, 9, 10, 11, 12, 13, 14, 15, 16, 17, 18, 19, or 20, etc.) membered heterocyclyl having 1-10 (e.g., 1, 2, 3, 4, 5, 6, 7, 8, 9, or 10, etc.) heteroatoms independently selected from nitrogen, oxygen and sulfur. In some embodiments, R is 3-20 (e.g., 3-20, 3-15, 3-10, or 3, 4, 5, 6, 7, 8, 9, 10, 11, 12, 13, 14, 15, 16, 17, 18, 19, or 20, etc.) membered heterocyclyl having 1-5 (e.g., 1, 2, 3, 4, or 5, etc.) heteroatoms independently selected from nitrogen, oxygen and sulfur. In some embodiments, R is 3-10 (e.g., 3, 4, 5, 6, 7, 8, 9, or 10, etc.) membered heterocyclyl having 1-5 (e.g., 1, 2, 3, 4, or 5, etc.) heteroatoms independently selected from nitrogen, oxygen and sulfur. In some embodiments, a heterocyclyl group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered heterocyclyl ring having 1-5 (e.g., 1, 2, 3, 4, or 5, etc.) heteroatoms independently selected from nitrogen, oxygen and sulfur. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur. In some embodiments, each heteroatom is independently selected from nitrogen, oxygen and sulfur.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₁₄ aryl-C₁₋₁₀ aliphatic, C₆₋₁₄ aryl-C₁₋₁₀ heteroaliphatic having 1-5 heteroatoms, C₁₋₁₀ aliphatic-C₆₋₁₄ aryl, C₁₋₁₀ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₀ aliphatic, 5-14 membered heteroaryl having 1-10 heteroatoms-C₁₋₁₀ heteroaliphatic having 1-5 heteroatoms, C₁₋₁₀ aliphatic-5-14 membered heteroaryl having 1-10 heteroatoms, C₁₋₁₀ heteroaliphatic having 1-5 heteroatoms-5-14 membered heteroaryl having 1-10 heteroatoms, C₂₋₂₀ biaryl having 0-10 heteroatoms wherein each aromatic unit is independently 5-10 membered and has 0-10 heteroatoms, and 3-15 membered heterocyclyl having 1-5 heteroatoms, or
two R groups are optionally and independently taken together to form a covalent bond or ═O, or:
two or more R groups on the same atom are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the atom, 0-5 (e.g., 0, 1, 2, 3, 4, or 5) heteroatoms; or
two or more R groups on two or more atoms are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the intervening atoms, 0-5 (e.g., 0, 1, 2, 3, 4, or 5) heteroatoms.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₁₄ aryl-C₁₋₁₀ aliphatic, C₆₋₁₄ aryl-C₁₋₁₀ heteroaliphatic having 1-5 heteroatoms, C₁₋₁₀ aliphatic-C₆₋₁₄ aryl, C₁₋₁₀ heteroaliphatic having 1-5 heteroatoms-C₆₋₁₄ aryl, 5-14 membered heteroaryl having 1-5 heteroatoms, 5-14 membered heteroaryl having 1-5 heteroatoms-C₁₋₁₀ aliphatic, 5-14 membered heteroaryl having 1-5 heteroatoms-C₁₋₁₀ heteroaliphatic having 1-5 heteroatoms, C₁₋₁₀ aliphatic-5-14 membered heteroaryl having 1-5 heteroatoms, C₁₋₁₀ heteroaliphatic having 1-5 heteroatoms-5-14 membered heteroaryl having 1-5 heteroatoms, C₂₋₂₀ biaryl having 0-10 heteroatoms, and 3-15 membered heterocyclyl having 1-5 heteroatoms, or two R groups are optionally and independently taken together to form a covalent bond or ═O, or: two or more R groups on the same atom are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the atom, 0-5 (e.g., 0, 1, 2, 3, 4, or 5) heteroatoms; or two or more R groups on two or more atoms are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14 or 15) membered, monocyclic, bicyclic or polycyclic ring having, in addition to the intervening atoms, 0-5 (e.g., 0, 1, 2, 3, 4, or 5) heteroatoms.

In some embodiments, each heteroatom is independently selected from oxygen, nitrogen, sulfur, phosphorus and silicon. In some embodiments, each heteroatom is independently selected from oxygen, nitrogen and sulfur.

In some embodiments, two R groups are optionally and independently taken together to form a covalent bond. In some embodiments, two R groups attached to neighboring atoms are optionally and independently taken together to form a covalent bond.

In some embodiments, two R groups are optionally and independently taken together with the atom to form an optionally substituted, 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered, monocyclic, bicyclic or polycyclic ring having, in addition to the atom, 0-10 (e.g., 0, 1, 2, 3, 4, 5, 6, 7, 8, 9, or 10, etc.) heteroatoms independently selected from oxygen, nitrogen, sulfur, phosphorus and silicon. In some embodiments, two or more R groups are optionally and independently taken together with their intervening atoms to form an optionally substituted, 3-30 (e.g., 3-25, 3-20, 3-15, 3-10, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered, monocyclic, bicyclic or polycyclic ring having, in addition to the intervening atoms, 0-10 (e.g., 0, 1, 2, 3, 4, 5, 6, 7, 8, 9, or 10, etc.) heteroatoms independently selected from oxygen, nitrogen, sulfur, phosphorus and silicon.

As described herein, in various instances, two or more R groups, or two or more groups that are or can be R (e.g., R^{s}, R′, etc.,), can be together with their intervening atom(s) to form an optionally substituted ring as described herein. In some embodiments, a formed ring is substituted (in addition to groups attached to the intervening atom(s). In some embodiments, a formed ring is unsubstituted. In some embodiments, a formed ring is 3-30, 3-25, 3-20, 3-15, 3-10, 3-8, 3-6, 5-6, or 3, 4, 5, 6, 7, 8, 9, 10, 11, 12, 13, 14, 15, 16, 17, 18, 19, 20, 21, 22, 23, 24, 25, 26, 27, 28, 29 or 30, etc.) membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is 11-membered. In some embodiments, a formed ring is 12-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-10 (e.g., 0, 1, 2, 3, 4, 5, 6, 7, 8, 9, or 10, etc.) heteroatoms independently selected from oxygen, nitrogen, sulfur, phosphorus and silicon. In some embodiments, a formed ring has, in addition to the intervening atom(s), 0-5 (e.g., 0, 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, R is a suitable group described in Table 1 to Table 7.

As described herein, many groups, moieties, etc. are independently optionally substituted. Those skilled in the art appreciate that various substituents are available and may be utilized in accordance with the present disclosure.

For example, in some embodiments, for optional substitution, each monovalent substituent, if any, e.g., on a substituted carbon atom, is independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₆ straight or branched alkylene)O—N(R^{∘})₂; or —(C₁₋₆ straight or branched alkylene)C(O)O—N(R^{∘})₂, wherein each R^{∘} may be substituted as defined herein and is independently hydrogen, C₁₋₂₀ (e.g., C₁₋₁₀, C₁₋₆, C₁₋₅, C₁₋₄, etc.) aliphatic, C₁₋₂₀ (e.g., C₁₋₁₀, C₁₋₆, C₁₋₅, C₁₋₄, etc.) heteroaliphatic having 1-5 (e.g., 1, 2, 3, 4 or 5) heteroatoms independently selected from nitrogen, oxygen, sulfur, silicon and phosphorus, —CH₂—(C₆₋₁₄ (e.g., C₆₋₁₀, C₆, etc.) aryl), —O(CH₂)₀₋₁(C₆₋₁₄ (e.g., C₆₋₁₀, C₆, etc.) aryl), —CH₂-(5-14 (e.g., 5-10, 5-6, 5, 6, 9, 10, 14, etc.) membered heteroaryl ring having 1-5 (e.g., 1, 2, 3, 4, or 5) heteroatoms independently selected from nitrogen, oxygen and sulfur), a 3-20 (e.g., 3-15, 3-10, 3-7, 3-6, 5-10, 5-6, etc.) membered, monocyclic, bicyclic, or polycyclic, saturated, partially unsaturated or aryl ring having 0-5 (e.g., 0, 1, 2, 3, 4, or 5) heteroatoms independently selected from nitrogen, oxygen, sulfur, silicon and phosphorus, or, notwithstanding the definition above, two independent occurrences of R^{∘}, taken together with their intervening atom(s), form a 3-20 (e.g., 3-15, 3-10, 3-7, 3-6, 5-10, 5-6, etc.) membered, monocyclic, bicyclic, or polycyclic, saturated, partially unsaturated or aryl ring having 0-5 (e.g., 0, 1, 2, 3, 4, or 5) heteroatoms independently selected from nitrogen, oxygen, sulfur, silicon and phosphorus, which may be substituted as defined below;
each monovalent substituent, if any, on R^{∘} (or the ring formed by taking two independent occurrences of R^{∘} together with their intervening atoms), is independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 3-6 (e.g., 4-6, 5-6, 3, 4, 5, 6, etc.) membered saturated, partially unsaturated, or aryl ring having 0-4 (e.g., 0, 1, 2, 3, or 4) heteroatoms independently selected from nitrogen, oxygen, and sulfur;
each divalent substituent, if any, e.g., on a substituted carbon atom, of R^{∘} are ═O and ═S;
each divalent substituent, if any, e.g., on a substituted carbon atom, is independently the following: ═O, ═S, ═NNR*₂, =NNHC(O)R*, =NNHC(O)OR*, =NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 3-6 (e.g., 4-6, 5-6, 3, 4, 5, 6, etc.) membered saturated, partially unsaturated, or aryl ring having 0-4 (e.g., 0, 1, 2, 3, or 4) heteroatoms independently selected from nitrogen, oxygen, and sulfur;
each divalent substituent, if any, that is bound to vicinal substituted atoms, e.g., carbon atoms, of an substituted group is —O(CR*₂)₂₋₃O—, wherein each independent occurrence of R* is selected from hydrogen, C₁₋₆ aliphatic which may be substituted as defined below, and an unsubstituted 3-6 (e.g., 4-6, 5-6, 3, 4, 5, 6, etc.) membered saturated, partially unsaturated, and aryl ring having 0-4 (e.g., 0, 1, 2, 3 or 4) heteroatoms independently selected from nitrogen, oxygen, and sulfur;
each substituent, if any, on the aliphatic group of R* is independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 (e.g., 0, 1, 2, 3, or 4) heteroatoms independently selected from nitrogen, oxygen, and sulfur;
each substituent, if any, on a nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 4-6, 5-6, etc.) membered saturated, partially unsaturated, or aryl ring having 0-4 (e.g., 0, 1, 2, 3, or 4) heteroatoms independently selected from nitrogen, oxygen, and sulfur, or, notwithstanding the definition above, two independent occurrences of Rf, taken together with their intervening atom(s) form an unsubstituted 3-12 (e.g., 3-10, 3-7, 3-6, 5-10, 5-7, 5-6, etc.) membered saturated, partially unsaturated, or aryl mono- or bicyclic ring having 0-4 (e.g., 0, 1, 2, 3, or 4) heteroatoms independently selected from nitrogen, oxygen, and sulfur; and
each substituent, if any, on the aliphatic group of R^{†} is independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁_aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

In some embodiments, for optional substitution, each monovalent substituent, if any, e.g., on a substituted carbon atom, is independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₆ straight or branched alkylene)O—N(R^{∘})₂; or —(C₁₋₆ straight or branched alkylene)C(O)O—N(R^{∘})₂, wherein each R^{∘} may be substituted as defined herein and is independently hydrogen, C₁₋₆ (e.g., C₁₋₅, C₁₋₄, etc.) aliphatic, C₁₋₆ (e.g., C₁₋₅, C₁₋₄, etc.) heteroaliphatic having 1-5 (e.g., 1, 2, 3, 4 or 5) heteroatoms independently selected from nitrogen, oxygen, sulfur, silicon and phosphorus, —CH₂—(C₆₋₁₀ (e.g., C₆, C₁₀, etc.) aryl), —O(CH₂)₀₋₁(C₆₋₁₀ (e.g., C₆, C₁₀, etc.) aryl), —CH₂-(5-10 (e.g., 5-6, 5, 6, 9, or 10) membered heteroaryl ring having 1-5 (e.g., 1, 2, 3, 4, or 5) heteroatoms independently selected from nitrogen, oxygen and sulfur), a 3-10 (e.g., 3-9, 3-8, 3-7, 3-6, 5-10, 5-6, etc.) membered, monocyclic, bicyclic, or polycyclic, saturated, partially unsaturated or aryl ring having 0-5 (e.g., 0, 1, 2, 3, 4, or 5) heteroatoms independently selected from nitrogen, oxygen, sulfur, silicon and phosphorus, or, notwithstanding the definition above, two independent occurrences of R^{∘}, taken together with their intervening atom(s), form a 3-10 (e.g., 3-9, 3-8, 3-7, 3-6, 5-10, 5-6, etc.) membered, monocyclic, bicyclic, or polycyclic, saturated, partially unsaturated or aryl ring having 0-5 (e.g., 0, 1, 2, 3, 4, or 5) heteroatoms independently selected from nitrogen, oxygen, sulfur, silicon and phosphorus, which may be substituted as defined below;
each monovalent substituent, if any, on R^{∘} (or the ring formed by taking two independent occurrences of R^{∘} together with their intervening atoms), is independently halogen, —(CH₂)₀₋₂R^{•}, -(haloR^{•}), —(CH₂)₀₋₂OH, —(CH₂)₀₋₂OR, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —NO₂, —SiR^{∘}₃, —OSiR^{∘}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C-4 aliphatic, —CH₂Ph, —O(CH₂)₀₋₁Ph, and a 3-6 (e.g., 4-6, 5-6, 3, 4, 5, 6, etc.) membered saturated, partially unsaturated, or aryl ring having 0-4 (e.g., 0, 1, 2, 3, or 4) heteroatoms independently selected from nitrogen, oxygen, and sulfur;
each divalent substituent, if any, e.g., on a substituted carbon atom, of R^{∘} are ═O and =S;
each divalent substituent, if any, e.g., on a substituted carbon atom, is independently the following: ═O, ═S, ═NNR*₂, =NNHC(O)R*, =NNHC(O)OR*, =NNHS(O)₂R*, ═NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 3-6 (e.g., 4-6, 5-6, 3, 4, 5, 6, etc.) membered saturated, partially unsaturated, or aryl ring having 0-4 (e.g., 0, 1, 2, 3, or 4) heteroatoms independently selected from nitrogen, oxygen, and sulfur;
each divalent substituent, if any, that is bound to vicinal substituted atoms, e.g., carbon atoms, of an substituted group is —O(CR*₂)₂₋₃O—, wherein each independent occurrence of R* is selected from hydrogen, C₁₋₆ aliphatic which may be substituted as defined below, and an unsubstituted 3-6 (e.g., 4-6, 5-6, 3, 4, 5, 6, etc.) membered saturated, partially unsaturated, and aryl ring having 0-4 (e.g., 0, 1, 2, 3 or 4) heteroatoms independently selected from nitrogen, oxygen, and sulfur;
each substituent, if any, on the aliphatic group of R* is independently halogen, —R^{•}, -(haloR^{•}), —OH, —OR*, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C-4 aliphatic, —CH₂Ph, —O(CH₂)₀₋₁Ph, or a 3-6 (e.g., 4-6, 5-6, etc.) membered saturated, partially unsaturated, or aryl ring having 0-4 (e.g., 0, 1, 2, 3, or 4) heteroatoms independently selected from nitrogen, oxygen, and sulfur;
each substituent, if any, on a nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 4-6, 5-6, etc.) membered saturated, partially unsaturated, or aryl ring having 0-4 (e.g., 0, 1, 2, 3, or 4) heteroatoms independently selected from nitrogen, oxygen, and sulfur, or, notwithstanding the definition above, two independent occurrences of Rf, taken together with their intervening atom(s) form an unsubstituted 3-12 (e.g., 3-10, 3-7, 3-6, 5-10, 5-7, 5-6, etc.) membered saturated, partially unsaturated, or aryl mono- or bicyclic ring having 0-4 (e.g., 0, 1, 2, 3, or 4) heteroatoms independently selected from nitrogen, oxygen, and sulfur; and
each substituent, if any, on the aliphatic group of Rf is independently halogen, —R^{•}, -(haloR^{•}), —OH, —OR*, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁_aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur.

As described herein, provided technologies can improve properties and/or activities, including reducing off-target effects, side effects, adverse reactions, etc., of bile acids and analogs and/or derivatives thereof that comprises 3-OH bonded to moiety A. In some embodiments, 3-OH is replaced by R¹ or R^{1a} as described herein, wherein R¹ or R^{1a} is not —OH. In some embodiments, 3-OH is replaced by R¹ where R¹ is —H or halogen. In some embodiments, R¹ is —H. In some embodiments, R¹ is halogen. In some embodiments, R¹ is —F. In some embodiments, 3-OH is replaced by R^{1a} where R^{1a} is —H or halogen. In some embodiments, R^{1a} is —H. In some embodiments, R^{1a} is halogen. In some embodiments, R^{1a} is —F. In some embodiments, the present disclosure provides improved compounds that provide high levels of activity and/or selectivity (e.g., for FXR and/or TGR5 over MRGPRX4) and/or reduced levels of off-target effects, side effects, adverse reactions, etc. of reference compounds (e.g., those comprising 3-OH bonded to moiety A). Certain examples are described below.

In some embodiments, a provided compound, e.g., a compound of formula I or a salt thereof, has the structure of a compound described in WO 2016/073767, e.g., a compound of formula (I) or a salt thereof (including stereoisomer, solvate, hydrate or combination thereof) as described in WO 2016/073767, wherein —OR₆ in WO 2016/073767 (in various embodiments, —OR₆ is —OH in WO 2016/073767) is replaced with R¹ or R^{1a} as described herein. In some embodiments, —OR₆ in WO 2016/073767 and the —H that is attached to the same carbon as —OR₆ are replaced with R¹ and R^{1a} as described herein, e.g., in some embodiments, both by —H.

In some embodiments, one of R² and R^{2a} is —H and the other is R₈ as described herein. In some embodiments, one of R³ and R^{3a} is —H and the other is —OR₇ as described herein. In some embodiments, one of R⁴ and R^{4a} is —H and the other is R₄ as described herein. In some embodiments, one of R¹⁴ and R^{14a} is —H and the other is R₅ as described herein. In some embodiments, R⁵ is —H. In some embodiments, R⁶ is —H. In some embodiments, R^{6a} is —H. In some embodiments, R⁷ is —H. In some embodiments, R^{1a} is —H. In some embodiments, R⁸ is —H. In some embodiments, R⁹ is R as described herein. In some embodiments, R¹³ is R as described herein. In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{s0}-L^{s1a}-L^{s1b}-C(O)-L^{s3}-N(R′)S(O)₂—, wherein each of L^{s0}, L^{s1a}, L^{s1b}, and R′ is independently as described herein, and L^{s3} is a covalent bond or —N(R′)C(R′)₂C(O)—. In some embodiments, L¹ is -L^{s0}-L^{s1a}-L^{s1b}-C(O)-L^{s3}-NHS(O)₂—. In some embodiments, L¹ is -L^{s0}-L^{s1a}-L^{s1b}-. In some embodiments, L¹ is -L^{s0}-L^{s1a}-L^{s1b}-C(O)—. In some embodiments, L¹ is -L^{s0}-L^{s1a}-L^{s1b}-C(O)-L^{s3}-. In some embodiments, L¹ is -L^{s0}-L^{s1a}-L^{s1b}-C(O)-L^{s3}-N(R′)—. In some embodiments, L¹ is -L^{s0}-L^{s1a}-L^{s1b}-C(O)-L^{s3}-NH—. In some embodiments, L^{s0} is optionally substituted —CH₂—. In some embodiments, L^{s0} is —C(R′)₂—, In some embodiments, L^{s0} is —CHR′—. In some embodiments, L^{s0} is —CH(CH₃)—. In some embodiments, L^{s0} is —(S)—CH(CH₃)—. In some embodiments, L^{s0} is —(R)—CH(CH₃)—. In some embodiments, L^{s1a} is optionally substituted C₁-C₅ alkylene. In some embodiments, L^{s1a} is —(CH₂)m-. In some embodiments, L^{s1b} is optionally substituted —CH₂—. In some embodiments, L^{s1b} is —C(R′)₂—. In some embodiments, L^{s1b} is —CHR′—. In some embodiments, L^{s1b} is —CH(CH₃)—. In some embodiments, L^{s1b} is —(R)—CH(CH₃)—. In some embodiments, L^{s1b} is —(S)—CH(CH₃)—. In some embodiments, L^{s3} is a covalent bond. In some embodiments, L^{s3} is —N(R′)C(R′)₂C(O)—. In some embodiments, L^{s3} is —N(R′)CHR′C(O)—. In some embodiments, L^{s3} is —NHCHR′C(O)—. In some embodiments, L^{s3} is —NHCH₂C(O)—. In some embodiments, L¹ is —CH(CH₃)—(CH₂)m-CH₂—. In some embodiments, L¹ is —CH(CH₃)—(CH₂)m-CH₂—C(O)—. In some embodiments, L¹ is —CH(CH₃)—(CH₂)m-CH₂—C(O)NH—. In some embodiments, L¹ is —CH(CH₃)—(CH₂)m-CH₂—C(O)NH—S(O)₂—. In some embodiments, L′ is —CH(CH₃)—(CH₂)m-CH₂—C(O)NH—CH₂—. In some embodiments, L′ is —CH(CH₃)—(CH₂)m-CH₂—C(O)NH—CH₂—C(O)—. In some embodiments, L¹ is —CH(CH₃)—(CH₂)m-CH₂—C(O)NH—CH₂—C(O)NH—. In some embodiments, L¹ is —CH(CH₃)—(CH₂)m-CH₂—C(O)NH—CH₂—C(O)NH—S(O)₂—. In some embodiments, L¹ is —CH(CH₃)—(CH₂)m-CH(CH₃)—C(O)—. In some embodiments, L¹ is —CH(CH₃)—(CH₂)m-CH(CH₃)—C(O)NH—. In some embodiments, L¹ is —CH(CH₃)—(CH₂)m-CH(CH₃)—C(O)NH—S(O)₂—. In some embodiments, L¹ is —CH(CH₃)—(CH₂)m-CH(CH₃)—C(O)NH—CH₂—. In some embodiments, L¹ is —CH(CH₃)—(CH₂)m-CH(CH₃)—C(O)NH—CH₂—C(O)—. In some embodiments, L¹ is —CH(CH₃)—(CH₂)m-CH(CH₃)—C(O)NH—CH₂—C(O)NH—. In some embodiments, L¹ is —CH(CH₃)—(CH₂)m-CH(CH₃)—C(O)NH—CH₂—C(O)NH—S(O)₂—. In some embodiments, —CH(CH₃)— at the end is S; in some embodiments, it is R. In some embodiments, —CH(CH₃)— in the middle is S; in some embodiments, it is R.

In some embodiments, R^{L} is —C(O)N(R′)C(R′)₂C(O)N(R′)S(O)₂R^{s}. In some embodiments, R^{L} is —C(O)N(R′)S(O)₂R^{s}. In some embodiments, R^{L} is —N(R′)S(O)₂R^{s}. In some embodiments, R^{L} is —NHS(O)₂R^{s}. In some embodiments, R^{L} is —S(O)₂R^{s}. In some embodiments, R^{L} is —W as described herein. In some embodiments, R^{L} is C(O)N(R′)C(R′)₂C(O)N(R′)S(O)₂R′. In some embodiments, R^{L} is —C(O)N(R′)S(O)₂R′. In some embodiments, R^{L} is —N(R′)S(O)₂R′. In some embodiments, R^{L} is —NHS(O)₂R′. In some embodiments, R^{L} is —S(O)₂R′. In some embodiments, R^{L} is —R′ as described herein.

In some embodiments, R^{L} is R^{s}. In some embodiments, R^{L} is R′. In some embodiments, R^{L} is R.

In some embodiments, a provided compound is a compound of formula VIII or a salt thereof:

In some embodiments, a provided compound is a compound of formula A-I or a salt thereof:

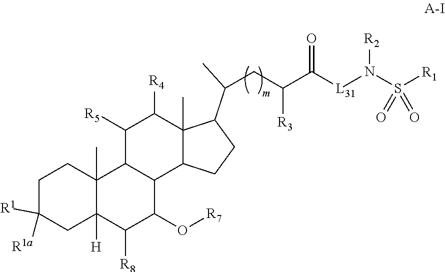

wherein:
   each of R¹ and R^{1a} is independently as described herein;
   R₁ is R₃₀ or R^{s} as described herein;
   R₃₀ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), alkylheteroaryl (e.g., C₁₋₁₂alkyl(5-14 membered heteroaryl having 1-10 heteroatoms)), and heteroarylalkyl (e.g., (5-14 membered heteroaryl having 1-10 heteroatoms)C₁₋₁₂alkyl);
L₃₁ is a covalent bond or —NR₁₁CHR₁₂C(O)—, wherein R₁₁ and R₁₂ are each independently R₃;
m is 0, 1, 2, or 3; and
each of R₂, R₃, R₄, R₅, R₇, R₈, R₉, and R₁₀ is independently R^{s} as described herein.

In some embodiments, a provided compound is a compound of formula A-I′ or a salt thereof:

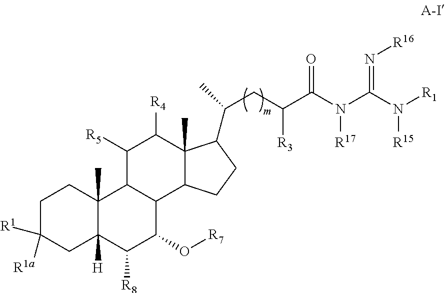

wherein each variable is independently as described herein.

In some embodiments:
each of R¹ and R^{1a} is independently as described herein;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), 5-14 membered heteroaryl having 1-10 heteroatoms, and alkylheteroaryl (e.g., C₁₋₁₂alkyl(5-14 membered heteroaryl having 1-10 heteroatoms));
R₉ and R₁₀ are each independently —H, or an optionally substituted group selected from C₁₋₈ alkyl, C₂₋₈ alkenyl, C₂₋₈ alkynyl, C₃₋₈ cycloalkyl, or R₉ and R₁₀ are taken together with the nitrogen to which they are attached to form an optionally substituted 3-15 membered, monocyclic, bicyclic or polycyclic heterocyclyl ring having 1-5 heteroatoms;
R₂ is —H or optionally substituted C₁₋₈ alkyl;
R₃ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl);
L₃₁ is a covalent bond or —NR₁₁CHR₁₂C(O)—, wherein R₁₁ and R₁₂ are each independently R₃;
m is 0, 1, 2, or 3;
R₄ is —H, —OH, —OSO₃H, —OAc or —OPO₃H₂;
R₅ is —H, halogen, —CN, —N₃, —OH, —OSO₃H, —OAc, —OPO₃H₂, —SR₂, or —NHR₂; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms;
R₇ is —H or a hydroxyl protecting group;
R⁸ is —H, halogen, or an optionally substituted group selected from C₁₋₈ alkyl, C₂₋₈ alkenyl, C₂₋₈ alkynyl, and C₃₋₈ cycloalkyl; and
each of R¹⁵, R¹⁶ and R¹⁷ is independently R^{s} as described herein.

In some embodiments, a provided compound is a compound of formula A-II-A or a salt thereof:

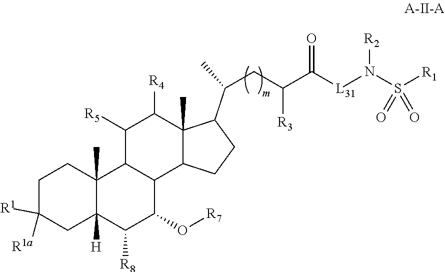

wherein each variable is independently as described herein.

In some embodiments, a provided compound is a compound of formula A-II-B or a salt thereof:

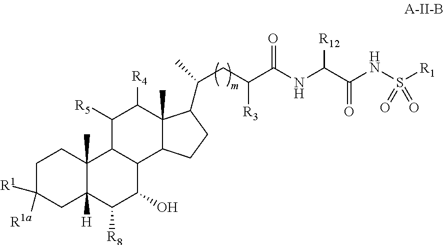

wherein each variable is independently as described herein.

In some embodiments, a provided compound is a compound of formula A-III-A or a salt thereof:

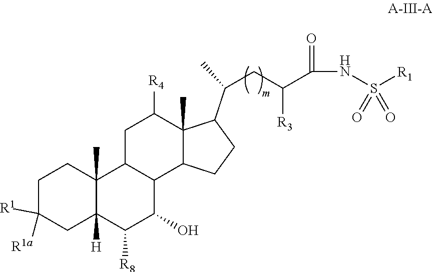

wherein each variable is independently as described herein.

In some embodiments, a provided compound is a compound of formula A-III-B or a salt thereof:

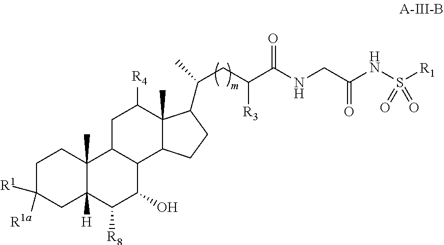

wherein each variable is independently as described herein.

In some embodiments, a provided compound is a compound of a formula below or a salt thereof:

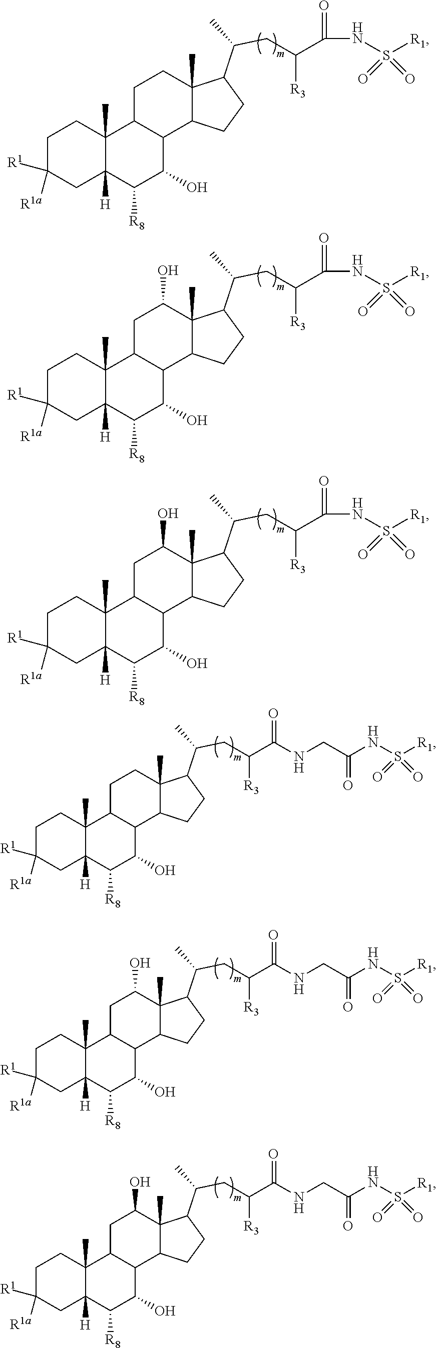

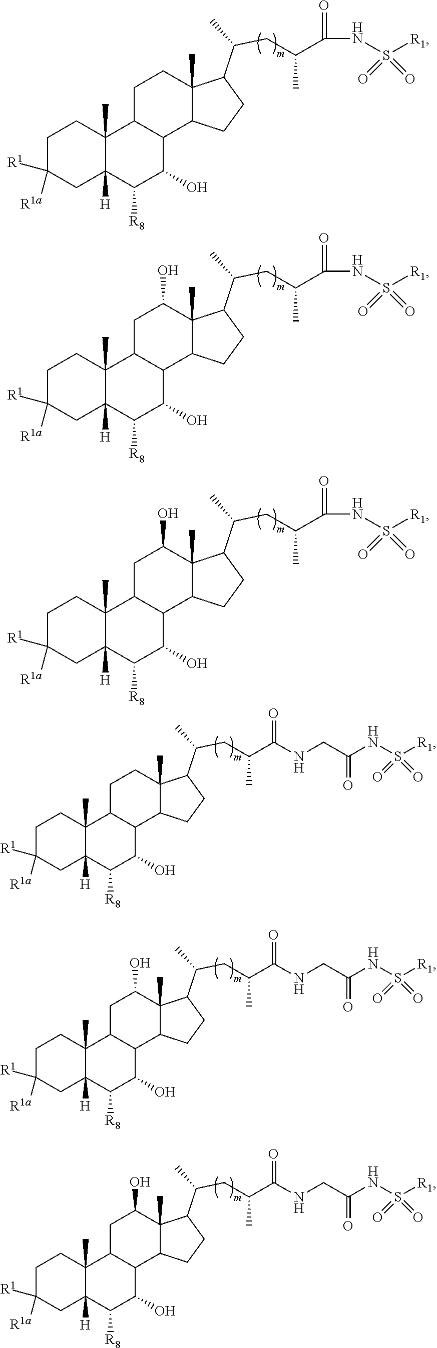

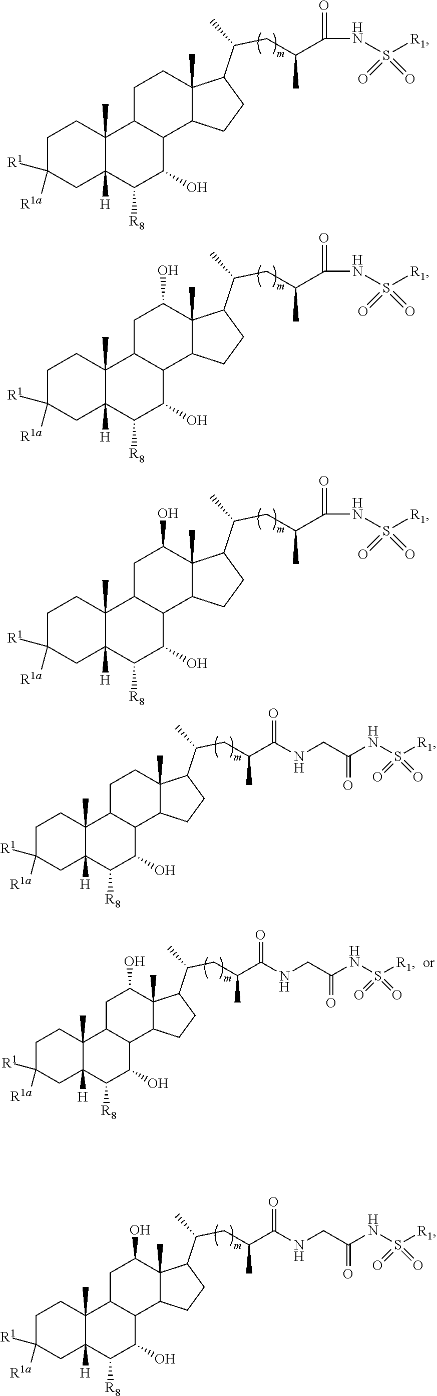

or a pharmaceutically acceptable salt thereof.

In some embodiments, a provided compound is a compound of formula A-IV-A or a salt

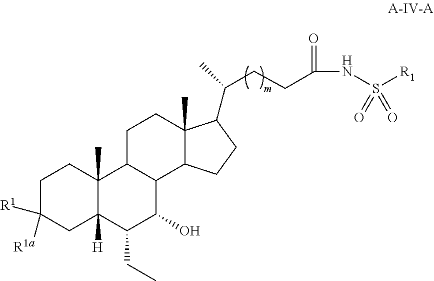

wherein each variable is independently as described herein.

In some embodiments, a provided compound is a compound of formula A-IV-B or a salt thereof:

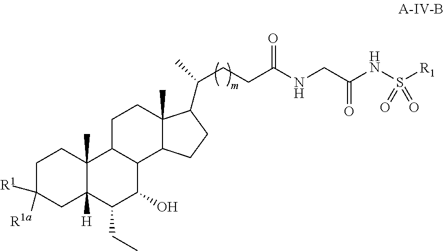

wherein each variable is independently as described herein.

In some embodiments, a provided compound is a compound of formula A-IV-A or a salt thereof:

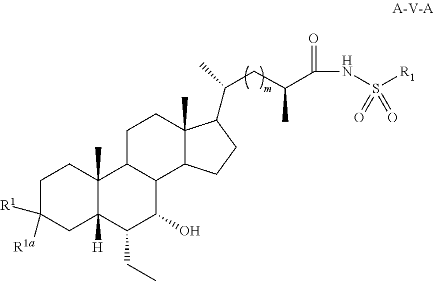

wherein each variable is independently as described herein.

In some embodiments, a provided compound is a compound of formula A-V-B or a salt thereof:

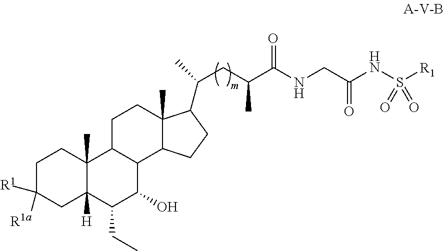

wherein each variable is independently as described herein.

In some embodiments, a provided compound is a compound of formula A-VI-A or a salt thereof:

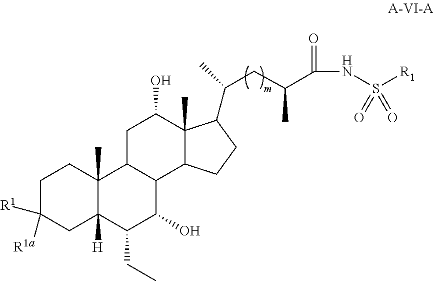

wherein each variable is independently as described herein.

In some embodiments, a provided compound is a compound of formula A-VI-B or a salt thereof:

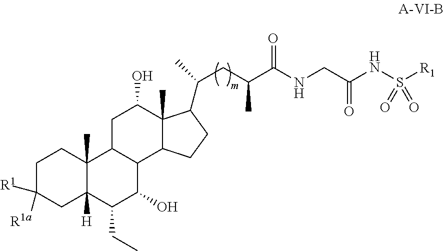

wherein each variable is independently as described herein.

In some embodiments, a provided compound is a compound selected from compounds 451-492 of WO 2016/073767, wherein its 3-OH and 3-H attached to moiety A are replaced with R¹ and R^{1a}, e.g., in some embodiments, both replaced with —H.

In some embodiments, R₁ is selected from the groups set forth below:

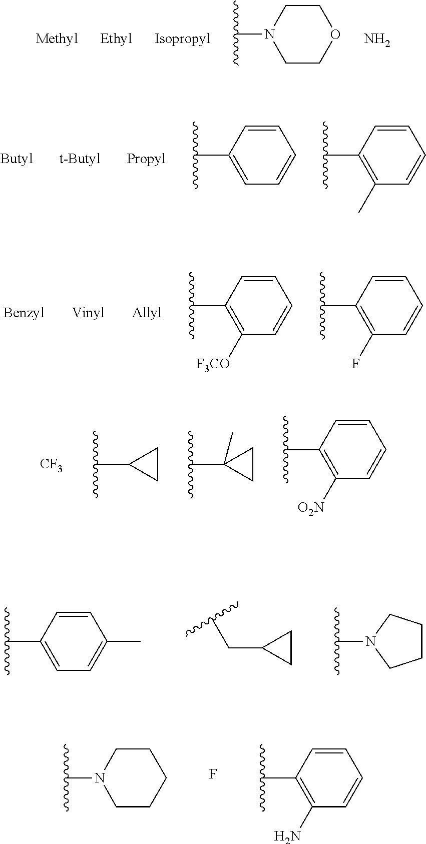

In some embodiments, m is 0. In some embodiments, m is 1. In some embodiments, m is 2.

In some embodiments, R₁ is selected from the groups set forth below:

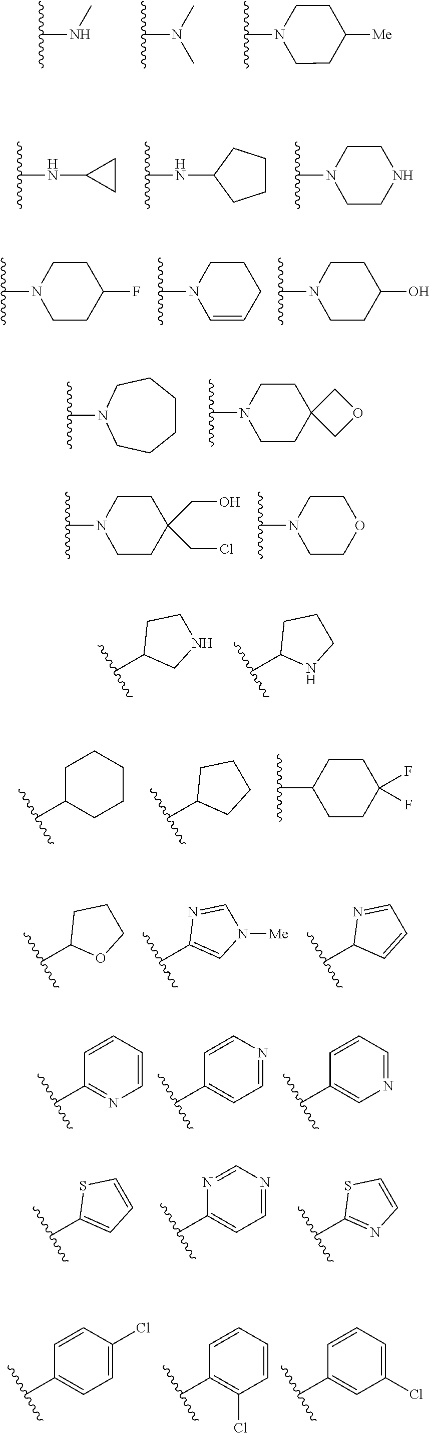

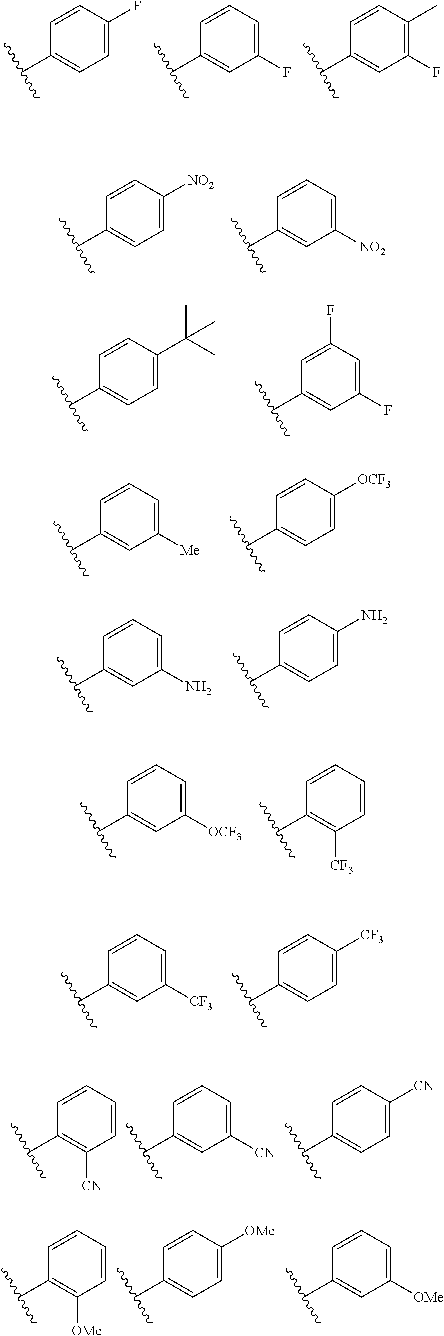

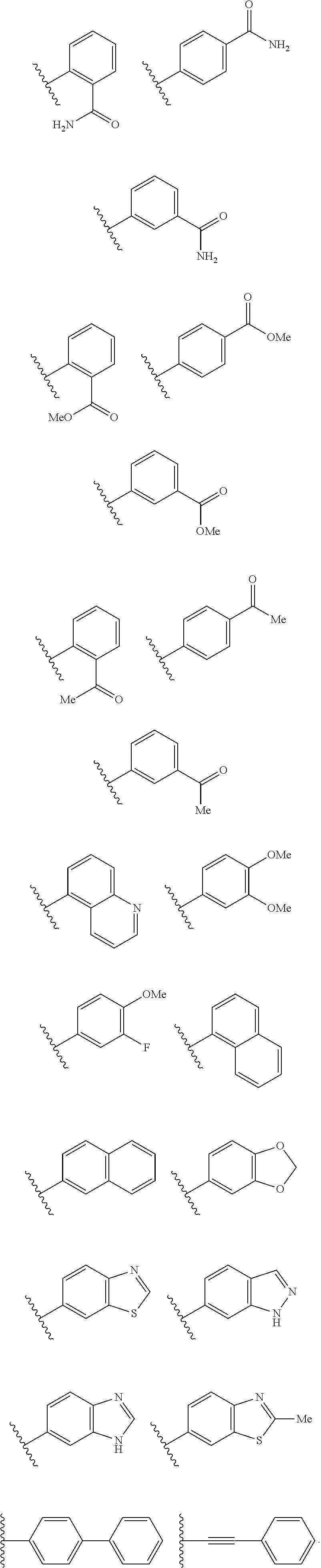

In some embodiments, m is 0. In some embodiments, m is 1. In some embodiments, m is 2.

In some embodiments, R₂ is —H. In some embodiments, R₃ is —H. In some embodiments, R₃ is methyl. In some embodiments, R₄ is —H. In some embodiments, R₄ is —OH. In some embodiments, R₅ is —H. In some embodiments, R₆ is —H. In some embodiments, R₇ is —H. In some embodiments, Rₛ is ethyl. In some embodiments, m is 0. In some embodiments, m is 1. In some embodiments, m is 2.

In some embodiments, R₂ is —H, R₃ is —H or methyl, R₄ is —H or —OH, R₅ is —H, R₆ is —H, R, is —H, Rₛ is ethyl, and m is 0, 1, or 2. In some embodiments, R₂ is —H, R₃ is —H or methyl, R₄ is —H, R₅ is —H, R₆ is —H, R₇ is —H, Rₛ is ethyl, and m is 0, 1, or 2. In some embodiments, R₂ is —H, R₃ is —H or methyl, R₄ is —OH, R₅ is —H, R₆ is —H, R₇ is —H, Rₛ is ethyl, and m is 0, 1, or 2.

Various additional embodiments of R₁, R₂, L₃₁ (e.g., those described for L), R₃, R₄, R₅, R₇, R₈, R₉ and R₁₀ are described in WO 2016/073767, either individually or in combination (e.g., as in compounds).

In some embodiments, a provided compound is a compound described in WO 2016/073767, wherein its 3-substitutents attached to moiety A are replaced with R¹ and R^{1a}, e.g., in some embodiments, both replaced with —H.

In some embodiments, a provided compound is a compound selected from compounds in Schemes 1-2, Examples 1, 93, 485, 486, 487, and 488, and Tables 1-10 of WO 2016/073767, wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2016/086115, e.g., a compound of formula (I) or a salt (including stereoisomer, solvate, hydrate or combination thereof) as described in WO 2016/086115, wherein —OR₄ in WO 2016/086115 (in various embodiments, —OR₄ is —OH in WO 2016/086115) is replaced with R^{1a} as described herein. In some embodiments, —OR₄ in WO 2016/086115 and the —H that is attached to the same carbon as —OR₆ are replaced with R¹ and R^{1a} as described herein, e.g., in some embodiments, both replaced with —H. In some embodiments, one of R² and R^{2a} is —H and the other is R₈ as described herein. In some embodiments, one of R³ and R^{3a} is —H and the other is —OR₇ as described herein. In some embodiments, one of R⁴ and R^{4a} is —H and the other is R₄ as described herein. In some embodiments, one of R¹⁴ and R^{14a} is —H and the other is R₅ as described herein. In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein. In some embodiments, R¹³ is R as described herein. In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{s0}-L^{s1a}-L^{s1b}-L^{s2}-, wherein L^{s2} is a covalent bond, —C(O)N(R′)— or —N(R′)—. In some embodiments, L^{s2} is a covalent bond. In some embodiments, L^{s2} is —C(O)N(R′)—. In some embodiments, L^{s2} is —C(O)NH—. In some embodiments, L^{s2} is —NH—. In some embodiments, L^{s0} is optionally substituted —CH₂—. In some embodiments, L^{s0} is —C(R′)₂—, In some embodiments, L^{s0} is —CHR′—. In some embodiments, L^{s0} is —CH(CH₃)—. In some embodiments, L^{s0} is —(S)—CH(CH₃)—. In some embodiments, L^{s0} is —(R)—CH(CH₃)—. In some embodiments, L^{s1a} is optionally substituted C₁-C₅ alkylene. In some embodiments, L^{s1a} is —(CH₂)m-. In some embodiments, L^{s1b} is optionally substituted —CH₂—. In some embodiments, L^{s1b} is —C(R′)₂—. In some embodiments, L^{s1b} is —CHR′—. In some embodiments, L^{s1b} is —CH(CH₃)—. In some embodiments, L^{s1b} is —(R)—CH(CH₃)—. In some embodiments, L^{s1b} is —(S)—CH(CH₃)—. In some embodiments, L¹ is —CH(CH₃)—(CH₂)m-CH₂—. In some embodiments, L¹ is —CH(CH₃)—(CH₂)m-CH₂—N(R′)—. In some embodiments, L¹ is —CH(CH₃)—(CH₂)m-CH₂—NH—. In some embodiments, L¹ is —CH(CH₃)—(CH₂)m-CH₂—C(O)N(R′)—. In some embodiments, L¹ is —CH(CH₃)—(CH₂)m-CH₂—C(O)NH—. In some embodiments, L¹ is —CH(CH₃)—(CH₂)m-CH(CH₃)—. In some embodiments, L¹ is —CH(CH₃)—(CH₂)m-CH(CH₃)—N(R′)—. In some embodiments, L¹ is —CH(CH₃)—(CH₂)m-CH(CH₃)—NH—. In some embodiments, L¹ is —CH(CH₃)—(CH₂)m-CH(CH₃)—C(O)N(R′)—. In some embodiments, L¹ is —CH(CH₃)—(CH₂)m-CH(CH₃)—C(O)NH—. In some embodiments, —CH(CH₃)— at the left end is S; in some embodiments, it is R. In some embodiments, —CH(CH₃)— in the middle or at the right end is S; in some embodiments, it is R.

In some embodiments, R^{L} is optionally substituted 5-14 (e.g., 5-10, 5-9, 1, 2, 3, 4, 5, 6, 7, 8, 9, 10, 11, 12, 13, or 14) heteroaryl having 1-5 (e.g., 1, 2, 3, 4, or 5) heteroatoms. In some embodiments, R^{L} is optionally substituted 5-membered heteroaryl. In some embodiments, R^{L} is optionally substituted 6-membered heteroaryl. In some embodiments, R^{L} is optionally substituted 3-15 (e.g., 3-10, 3-9, 1, 2, 3, 4, 5, 6, 7, 8, 9, 10, 11, 12, 13, 14 or 15) membered heterocyclyl having 1-5 (e.g., 1, 2, 3, 4, or 5) heteroatoms. In some embodiments, there is one heteroatom. In some embodiments, there are two or more heteroatoms. In some embodiments, there are two heteroatoms. In some embodiments, there are three heteroatoms. In some embodiments, there are four heteroatoms. In some embodiments, there are five heteroatoms. In some embodiments, at least one heteroatom is nitrogen. In some embodiments, each heteroatom is nitrogen. In some embodiments, R^{L} is

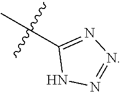 [SMILES: *c1nnn[nH]1]

In some embodiments, a provided compound is a compound of formula B—I or a salt thereof:

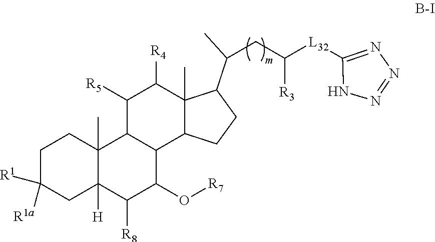

wherein:
   L₃₂ is a covalent bond, —C(O)NH— or —NH—; and
   each other variable is independently as described herein.

In some embodiments, a provided compound is a compound of formula B—I′ or a salt thereof:

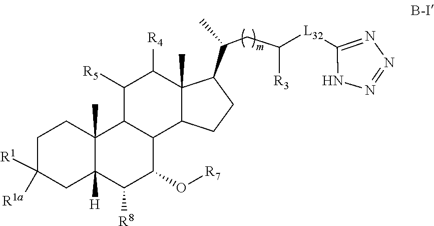

wherein each variable is independently as described herein.

In some embodiments:
each of L₃₂, R¹ and R^{1a} is independently as described herein;
R₃ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl);
m is 0, 1, 2, or 3;
R₄ is —H, —OH, —OSO₃H, —OAc or —OPO₃H₂;
R₅ is —H, halogen, —CN, —N₃, —OH, —OSO₃H, —OAc, —OPO₃H₂, —SR₃, or —NHR₃; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms;
R₇ is —H or a hydroxyl protecting group; and
R⁸ is —H, halogen, or an optionally substituted group selected from C₁₋₈ alkyl, C₂₋₈ alkenyl, C₂₋₈ alkynyl, and C₃₋₈ cycloalkyl.

In some embodiments, a provided compound is a compound of formula B-IA or a salt thereof:

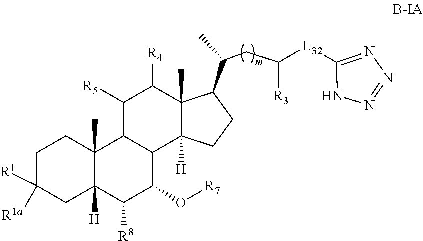

wherein each variable is independently as described herein.

In some embodiments, a provided compound is a compound of formula B-II or a salt thereof:

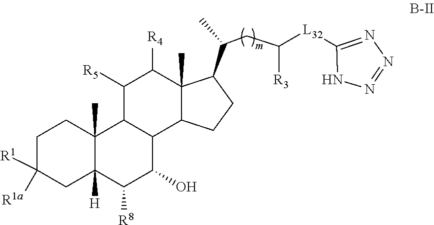

wherein each variable is independently as described herein.

In some embodiments, a provided compound is a compound of formula B—III or a salt thereof:

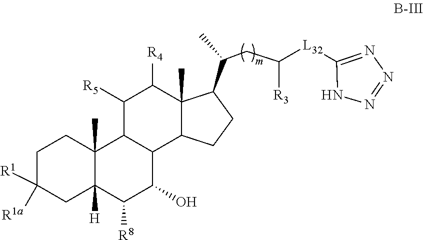

wherein each variable is independently as described herein. In some embodiments, a provided compound has a structure selected from below or a salt thereof:

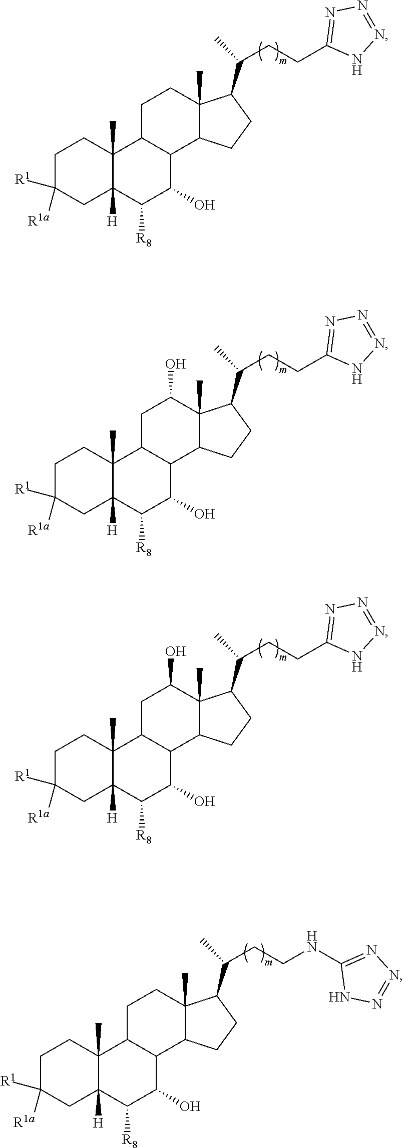

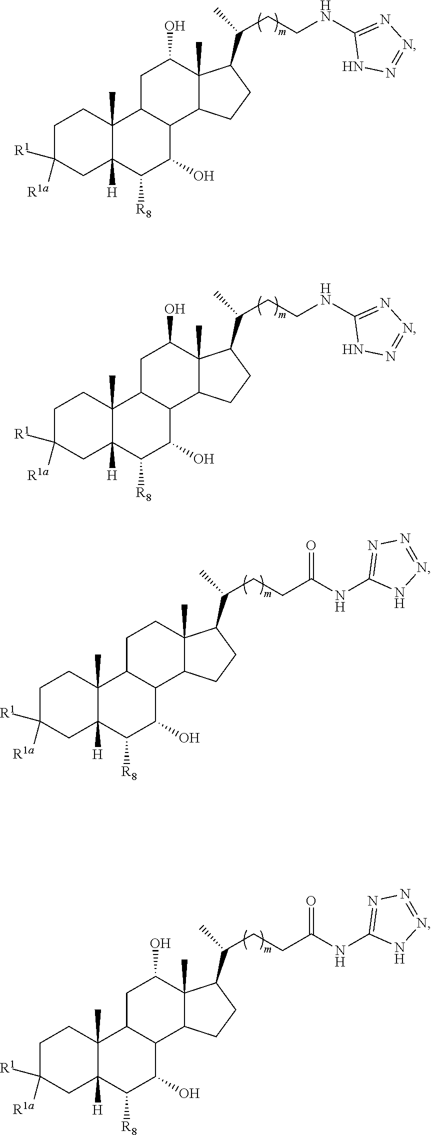

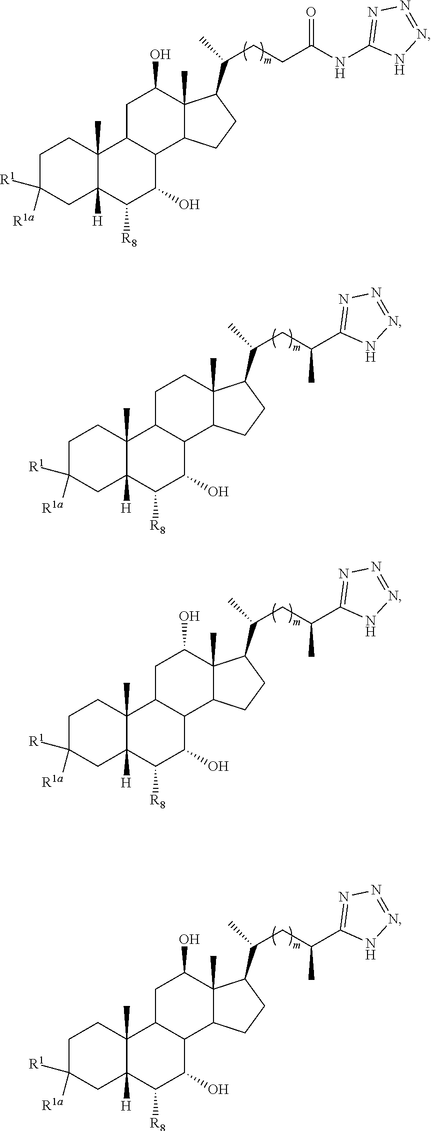

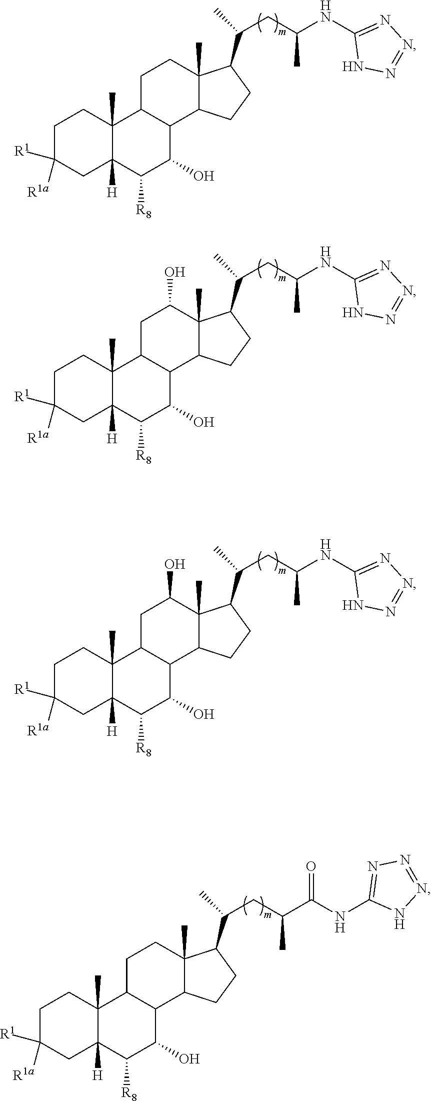

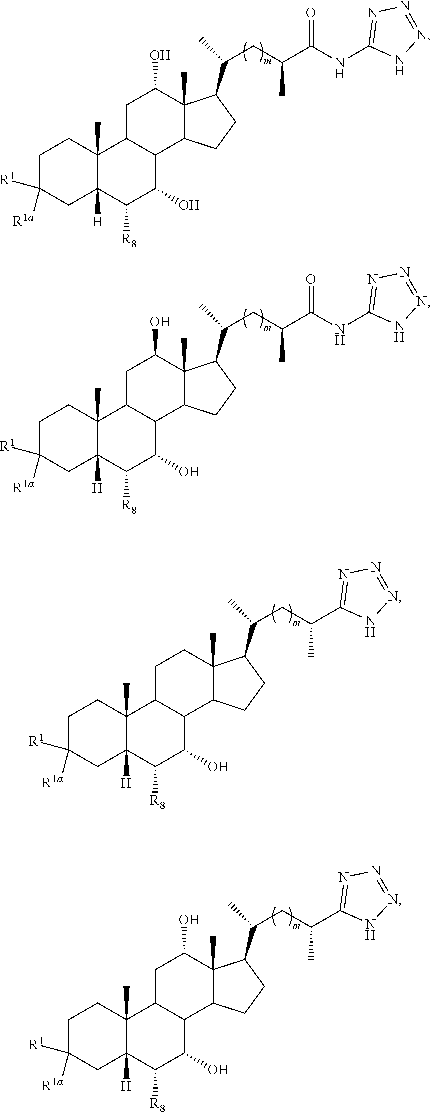

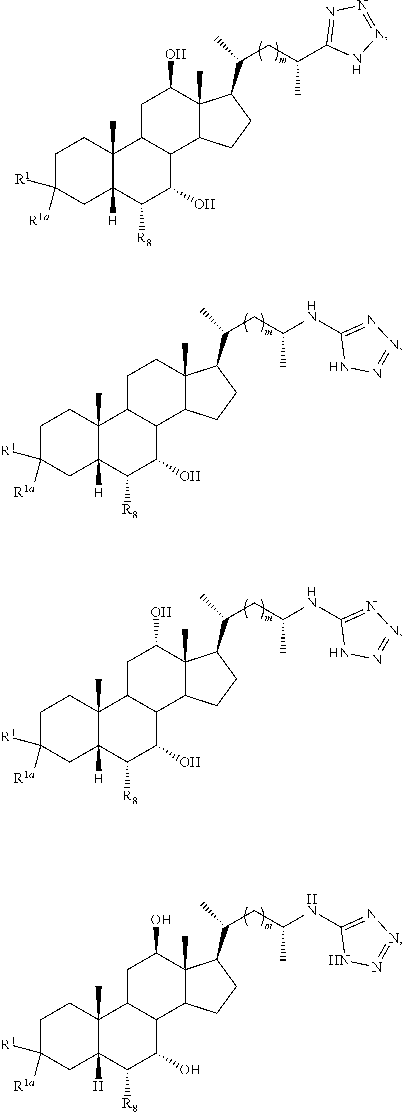

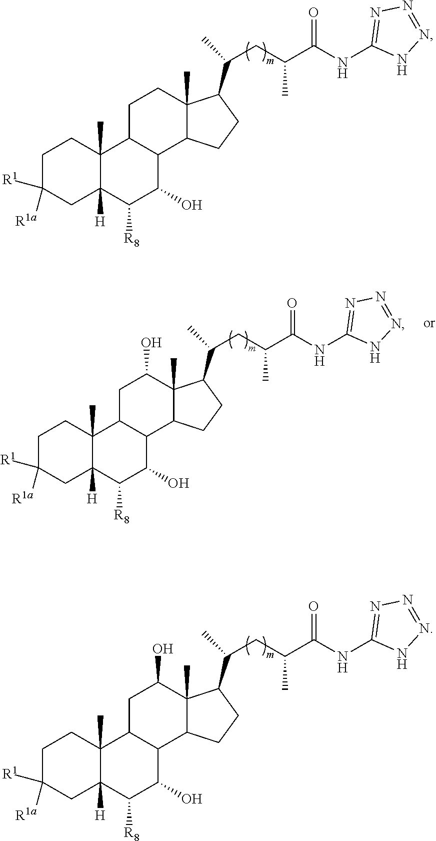

In some embodiments, a provided compound is a compound of formula B—IV-A or a salt thereof.

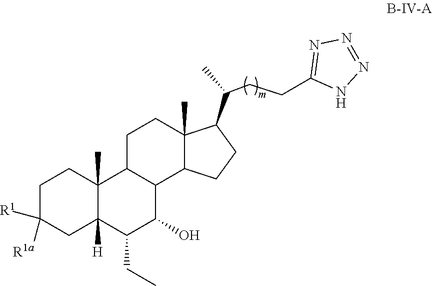

wherein each variable is independently as described herein.

In some embodiments, a provided compound is a compound of formula B-IV-B or a salt thereof:

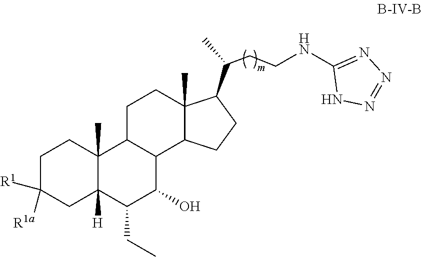

wherein each variable is independently as described herein.

In some embodiments, a provided compound is a compound of formula B—V-A or a salt thereof:

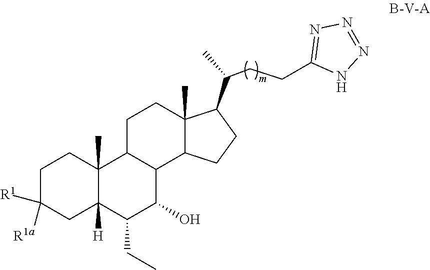

wherein each variable is independently as described herein.

In some embodiments, a provided compound is a compound of formula B-V-B or a salt thereof:

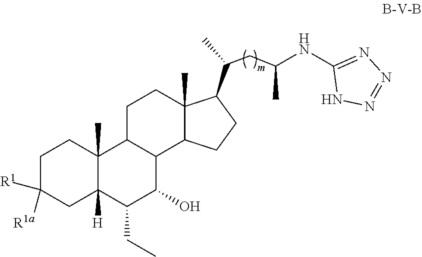

wherein each variable is independently as described herein.

In some embodiments, a provided compound is a compound of formula B—VI-A or a salt thereof:

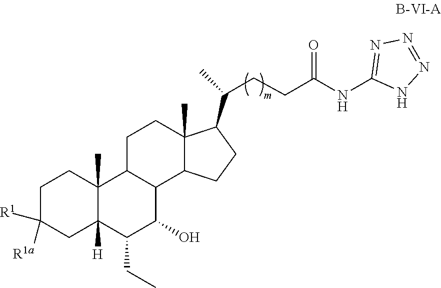

wherein each variable is independently as described herein.

In some embodiments, a provided compound is a compound of formula B-VI-B or a salt thereof:

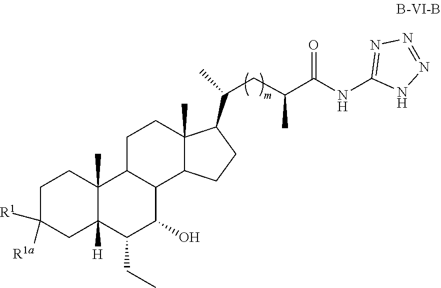

wherein each variable is independently as described herein.

In some embodiments, a provided compound is a compound of formula B—VII-A or a salt thereof:

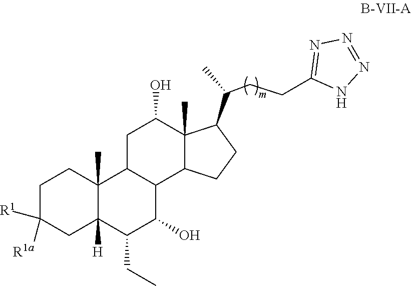

wherein each variable is independently as described herein.

In some embodiments, a provided compound is a compound of formula B-VII-B or a salt thereof:

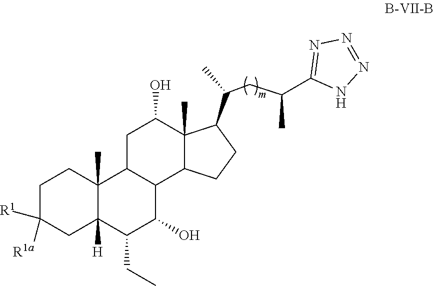

wherein each variable is independently as described herein.

In some embodiments, a provided compound is a compound of formula B—VIII-A or a salt

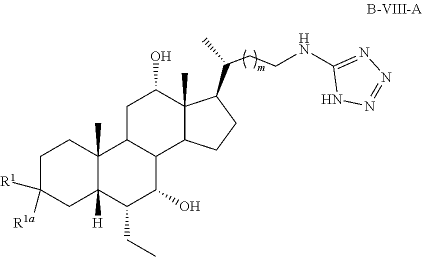

wherein each variable is independently as described herein.

In some embodiments, a provided compound is a compound of formula B-VIII-B or a salt thereof:

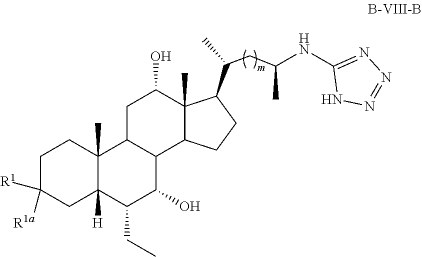

wherein each variable is independently as described herein.

In some embodiments, a provided compound is a compound of formula B—IX-A or a salt thereof:

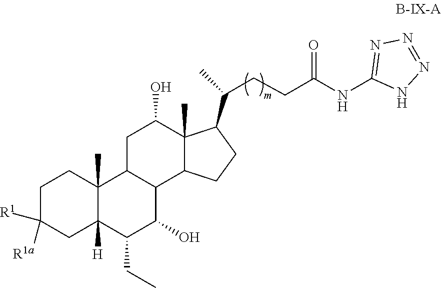

wherein each variable is independently as described herein.

In some embodiments, a provided compound is a compound of formula B—IX—B or a salt thereof:

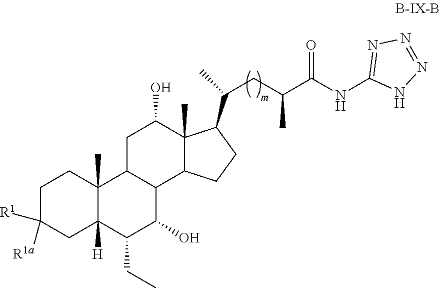

wherein each variable is independently as described herein.

In some embodiments, R₃ is —H or methyl. In some embodiments, R₃ is —H. In some embodiments, R₃ is methyl. In some embodiments, R₄ is —H. In some embodiments, R₄ is —OH. In some embodiments, R₅ is —H. In some embodiments, R₈ is C₁₋₄ alkyl. In some embodiments, R₈ is ethyl. In some embodiments, m is 0, 1 or 2. In some embodiments, m is 0. In some embodiments, m is 1. In some embodiments, m is 2.

Various additional embodiments of L₃₂, R₃, R₄, R₅, R₇, and R₈ are described in WO 2016/086115 (e.g., described as X, R₁, R₂, R₃, R₅, and R₆, respectively, in WO 2016/086115), either individually or in combination (e.g., as in compounds).

In some embodiments, a provided compound is a compound described in WO 2016/086115, wherein its 3-substitutents attached to moiety A are replaced with R¹ and R^{1a}, e.g., in some embodiments, both replaced with —H.

In some embodiments, a provided compound is a compound selected from compounds in Schemes 1-5 and Examples 1-6 of WO 2016/086115, wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2016/086134 or WO 2016/086218, e.g., a compound of formula (I) or (I′) or a salt (including stereoisomer, solvate, hydrate or combination thereof) as described in WO 2016/086134 or WO 2016/086218, wherein —OR₅ in WO 2016/086134 or WO 2016/086218 (in various embodiments, —OR₅ is —OH in WO 2016/086134 or WO 2016/086218) is replaced with R^{1a} as described herein. In some embodiments, —OR₅ in WO 2016/086134 or WO 2016/086218 and the —H that is attached to the same carbon as such —OR₅ are replaced with R¹ and R^{1a} as described herein, e.g., in some embodiments, both replaced with —H. In some embodiments, one of R² and R^{2a} is —H and the other is R₈ as described herein. In some embodiments, one of R³ and R^{3a} is —H and the other is —OR₇ as described herein. In some embodiments, one of R⁴ and R^{4a} is —H and the other is R₄ as described herein. In some embodiments, one of R¹⁴ and R^{14a} is —H and the other is R₅ as described herein. In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein. In some embodiments, R¹³ is R as described herein. In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, in a provided compound L¹ is -L^{s0}-L′-, wherein L^{s0} is a covalent bond, or an optionally substituted methylene which is optionally replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—; L¹ is -L^{s1}-L^{s2}-; L^{s1} is a covalent bond, or an optionally substituted, bivalent C₁-C₆ aliphatic or heteroaliphatic group having 1-5 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—; and L^{s2} is a covalent bond, or an optionally substituted, bivalent C₁-C₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—.

In some embodiments, L^{s0} is —C(R′)₂—. In some embodiments, L^{s0} is —CH(CH₃)—. In some embodiments, L^{s1} is optionally substituted C₁-C₆ alkylene. In some embodiments, L^{s1} is C₁-C₆ alkylene. In some embodiments, L^{s1} is —(CH₂)ₘ—CH₂— as described herein. In some embodiments, L^{s1} is —(CH₂)ₘ— as described herein. In some embodiments, -L^{s0}-L^{s1}-is —C(R′)₂—(CH₂)m-. In some embodiments, -L^{s0}-L^{s1}-is —CHCH₃—(CH₂)ₘ. In some embodiments, L^{s2} is bonded to R^{L}. In some embodiments, L^{s2} is a covalent bond. In some embodiments, at least one unit of L^{s2} is independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, at least one unit of L^{s2} is independently replaced with —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{s2} is —C(R′)₂—. In some embodiments, L^{s2} is —CHR′—. In some embodiments, L^{s2} is optionally substituted —CH₂—. In some embodiments, L^{s2} is —CH₂—. In some embodiments, L^{s2} is —N(R′)C(O)N(R′)S(O)₂N(R′)—. In some embodiments, L^{s2} is —N(R′)C(O)N(R′)—. In some embodiments, L^{s2} is —N(R′)C(NR′)N(R′)—. In some embodiments, L^{s2} is —N(R′)C(O)N(R′)S(O)₂—. In some embodiments, L^{s2} is —N(R′)C(NR′)NR'S(O)₂—. In some embodiments, L^{s2} is —N(R′)C(NR′)NR'S(O)₂—. In some embodiments, L^{s2} is —N(R′)C(O)NR'S(O)₂—. In some embodiments, L^{s2} is —N(R′)C(O)N(R′)—. In some embodiments, L^{s2} is —NHC(O)NHS(O)₂NH—. In some embodiments, L^{s2} is —NHC(O)NH—. In some embodiments, L^{s2} is —N(CH₃)C(O)NH—. In some embodiments, L^{s2} is —NHC(NH)NH—. In some embodiments, L^{s2} is —N(CH₃)C(NH)NH—. In some embodiments, L^{s2} is —NHC(O)NHS(O)₂—. In some embodiments, L^{s2} is —N(CH₃)C(O)NHS(O)₂—. In some embodiments, L^{s2} is —NHC(NH)NHS(O)₂—. In some embodiments, L^{s2} is —NHC(NH)NHS(O)₂—. In some embodiments, L^{s2} is —N(CH₃)C(NH)NHS(O)₂—. In some embodiments, L^{s2} is or comprises —NHC(O)NH—. In some embodiments, L¹ is —CHR′—(CH₂)ₘ—CHR′—. In some embodiments, L¹ is —CH(CH₃)—(CH₂)ₘ—C(R′)—. In some embodiments, L¹ is -L^{s0}-(CH₂)ₘ-L^{s2}-, wherein each of L^{s0} and L^{s2} is independently optionally substituted —CH₂—. In some embodiments, L¹ is -L^{s0}-(CH₂)ₘ—CH₂—, wherein L^{s0} is optionally substituted —CH₂—. In some embodiments, L^{s0} is —CH(CH₃)—.

In some embodiments, L¹ is -L^{s0}-L^{s1}-L^{s2}-. In some embodiments, L^{s0} is optionally substituted —CH₂—. In some embodiments, L^{s0} is —C(R)₂—. In some embodiments, L^{s0} is —CHR′—. In some embodiments, L⁰ is —CH₂—. In some embodiments, L^{s0} is —CH(CH₃)—. In some embodiments, L^{s0} is optionally substituted —CH₂—, and L^{s1} is optionally substituted C₁-C₆ alkylene. In some embodiments, L¹ is -L^{s0}-L^{s1}-L^{s2}-, wherein L^{s0} is optionally substituted —CH₂—, L^{s1} is C₁-C₆ alkylene. In some embodiments, L¹ is -L^{s0}-L^{s1a}-L^{s1b}-L^{s2}-. In some embodiments, L^{s1} is -L^{s1a}-L^{s1b}-, wherein L^{s1a} is a covalent bond, or an optionally substituted, bivalent C₁-C₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—; and L^{s1b} is a covalent bond, or an optionally substituted methylene which is optionally replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—. In some embodiments, L^{s1a} is optionally substituted C₁-C₅ alkylene. In some embodiments, L^{s1a} is C₁-C₅ alkylene. In some embodiments, L^{s1b} is optionally substituted —CH₂—. In some embodiments, L^{s1b} is —C(R)₂—. In some embodiments, L^{s1b} is —CHR′—. In some embodiments, L^{s1b} is —CH₂—. In some embodiments, L^{s1b} is —CH(CH₃)—.

In some embodiments, L¹ is -L^{s0}-L^{s1}-L^{s2}-, wherein L^{s0} is optionally substituted —CH₂—, L^{s1} is an optionally substituted bivalent C₁₋₇ aliphatic group, and L^{s2} is optionally substituted —CH₂—. In some embodiments, L¹ is -L^{s0}-L^{s1}-L^{s2}-, wherein L^{s0} is —C(R′)₂—, L^{s1} is an optionally substituted bivalent C₁₋₇ aliphatic group, and L^{s2} is —C(R′)₂—. In some embodiments, L^{s0} is —CHR′—. In some embodiments, L^{s0} is (S)—CH(R′)—, wherein R′ is not —H. In some embodiments, L^{s0} is (R)—CH(R′)—, wherein R′ is not —H. In some embodiments, R′ is optionally substituted C₁₋₆ aliphatic. In some embodiments, R′ is optionally substituted C₁₋₆ alkyl. In some embodiments, R′ is —CH₃. In some embodiments, L^{s1} is optionally substituted C₁₋₆ alkylene. In some embodiments, L^{s1} is optionally substituted —(CH₂)₁₋₆—. In some embodiments, L^{s1} is —(CH₂)₁₋₆—. In some embodiments, L^{s1} is —(CH₂)m-. In some embodiments, m is 0. In some embodiments, m is 1. In some embodiments, m is 2. In some embodiments, m is 3. In some embodiments, m is 4. In some embodiments, m is 5. In some embodiments, L^{s2} is —CHR′—. In some embodiments, R′ is —H and L^{s2} is —CH₂—. In some embodiments, R′ is not —H. In some embodiments, L^{s2} is (S)—CH(R′)—, wherein R′ is not —H. In some embodiments, L^{s2} is (R)—CH(R′)—, wherein R′ is not —H. In some embodiments, R′ is optionally substituted C₁₋₆ aliphatic. In some embodiments, R′ is optionally substituted C₁₋₆ alkyl. In some embodiments, R′ is —CH₃. In some embodiments, L¹ is —CHR′—(CH₂)ₘ—CH₂—. In some embodiments, L¹ is —CH(CH₃)—(CH₂)ₘ—CH₂—. In some embodiments, L¹ is (R)—CH(CH₃)—(CH₂)ₘ—CH₂—. In some embodiments, L¹ is (S)—CH(CH₃)—(CH₂)ₘ—CH₂—. In some embodiments, L¹ is —CHR′—(CH₂)ₘ—CHR′—. In some embodiments, L¹ is —CH(CH₃)—(CH₂)ₘ—CH(CH₃)—. In some embodiments, L¹ is (R, R)—CH(CH₃)—(CH₂)ₘ—CH(CH₃)—. In some embodiments, L¹ is (R, S)—CH(CH₃)—(CH₂)ₘ—CH(CH₃)—. In some embodiments, L¹ is (S, S)—CH(CH₃)—(CH₂)ₘ—CH(CH₃)—. In some embodiments, L¹ is (S, R)—CH(CH₃)—(CH₂)ₘ—CH(CH₃)—.

In some embodiments, L¹ is -L^{s0}-L^{s1}-L^{s2}-, wherein L^{s0} is optionally substituted —CH₂— and L^{s1} is an optionally substituted bivalent C₁₋₇ aliphatic group. In some embodiments, L¹ is -L^{s0}-L^{s1}-L^{s2}-, wherein L^{s0} is —C(R′)₂— and L^{s1} is an optionally substituted bivalent C₁₋₇ aliphatic group. In some embodiments, L¹ is —CHR′—(CH₂)ₘ—CHR′-L^{s2}-. In some embodiments, L¹ is —CHR′—(CH₂)ₘ—CH₂-L^{s2}-. In some embodiments, L¹ is -L^{s0}-(CH₂)ₘ—CHR′-L^{s2}-, wherein L^{s0} is optionally substituted —CH₂—. In some embodiments, L¹ is -L^{s0}-(CH₂)ₘ—CH₂-L^{s2}-, wherein L^{s0} is optionally substituted —CH₂—. In some embodiments, L^{s0} is —CH(CH₃)—. In some embodiments, m is 0. In some embodiments, m is 1. In some embodiments, m is 2.

In some embodiments, L^{s2} is —N(R′)C(O)N(R′)S(O)₂—. In some embodiments, L^{s2} is —NHC(O)NHS(O)₂—. In some embodiments, L^{s2} is —N(R′)C(S)N(R′)S(O)₂—. In some embodiments, L^{s2} is —NHC(S)NHS(O)₂—. In some embodiments, L^{s2} is —N(R′)C(O)C(O)N(R′)S(O)₂—. In some embodiments, L^{s2} is —NHC(O)C(O)NHS(O)₂—. In some embodiments, L^{s2} is —N(R′)S(O)₂—. In some embodiments, L^{s2} is —NHS(O)₂—. In some embodiments, L^{s2} is —N(R′)C(O)N(R′)—. In some embodiments, L^{s2} is —NHC(O)NH—. In some embodiments, L^{s2} is —N(R′)C(NR′)N(R′)—. In some embodiments, L^{s2} is —NHC(NR′)NH—. In some embodiments, L^{s2} is —N(R′)C(NR′)N(R′)S(O)₂—. In some embodiments, L^{s2} is —NHC(NR′)NHS(O)₂—.

In some embodiments, L^{s2} is —N(R′)C(O)N(R′)S(O)₂-L″-. In some embodiments, L^{s2} is —NHC(O)NHS(O)₂-L″-. In some embodiments, L^{s2} is —N(R′)C(S)N(R′)S(O)₂-L″-. In some embodiments, L^{s2} is —NHC(S)NHS(O)₂-L″-. In some embodiments, L^{s2} is —N(R′)C(O)C(O)N(R′)S(O)₂-L″-. In some embodiments, L^{s2} is —NHC(O)C(O)NHS(O)₂-L″-. In some embodiments, L^{s2} is —N(R′)S(O)₂-L″-. In some embodiments, L^{s2} is —NHS(O)₂-L″-. In some embodiments, L^{s2} is —N(R′)C(O)N(R′)-L″-. In some embodiments, L^{s2} is —NHC(O)NH-L″-. In some embodiments, L^{s2} is —N(R′)C(NR′)N(R′)-L″-. In some embodiments, L^{s2} is —NHC(NR′)NH-L″-. In some embodiments, L^{s2} is —N(R′)C(NR′)N(R′)S(O)₂-L″-. In some embodiments, L^{s2} is —NHC(NR′)NHS(O)₂-L″-. Various embodiments of L″ are described herein. For example, in some embodiments, L″ is optionally substituted —CH₂—CH₂—. In some embodiments, L″ is —CH₂-Cy- wherein the —CH₂— is optionally substituted. In some embodiments, L″ is —C(R′)₂-Cy-. In some embodiments, L″ is —CHR′-Cy-. In some embodiments, L″ is optionally substituted —CH═CH—. In some embodiments, L: is -Cy-. In some embodiments, L″ is -Cy-Cy-. In some embodiments, L″ is —N(R′)—. In some embodiments, L″ is —NH—. In some embodiments, L″ is —CH₂—N(R′)— wherein the —CH₂— is optionally substituted. In some embodiments, L″ is —C(R′)₂-Cy-. In some embodiments, L″ is —CHR′-Cy-. In some embodiments, -Cy- is optionally substituted 3-10 membered cycloalkyl, e.g., in some embodiments, -Cy-is

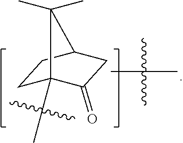

In some embodiments, -Cy- is an optionally substituted monocyclic 3-, 4-, 5-, 6-, or 7-membered ring having 0-2 heteroatoms. In some embodiments, -Cy- is optionally substituted phenylene. In some embodiments, -Cy- is phenylene. In some embodiments, -Cy- is an optionally substituted bivalent heteroaryl ring having 1-4 heteroatoms. In some embodiments, -Cy- is an optionally substituted monocyclic 5- or 6-membered bivalent heteroaryl ring having 1-4 heteroatoms. In some embodiments, -Cy- is an optionally substituted bicyclic 9- or 10-membered bivalent heteroaryl ring having 1-4 heteroatoms.

In some embodiments, L¹ is —CH(CH₃)(CH₂)mCH₂NHC(O)NHS(O)₂—. In some embodiments, L¹ is —CH(CH₃)(CH₂)mCH₂NHC(NH)NHS(O)₂—. In some embodiments, L¹ is —CH(CH₃)(CH₂)mCH₂N(CH₃)C(O)NHS(O)₂—. In some embodiments, L¹ is —CH(CH₃)(CH₂)mCH₂N(CH₃)C(NH)NHS(O)₂—. In some embodiments, L¹ is —CH(CH₃)(CH₂)mCH₂NHS(O)₂—. In some embodiments, L¹ is —CH(CH₃)(CH₂)mCH₂N(CH₃)S(O)₂—. In some embodiments, L¹ is —CH(CH₃)(CH₂)mCH₂NHC(O)NH—. In some embodiments, L¹ is —CH(CH₃)(CH₂)mCH₂NHC(NH)NH—. In some embodiments, L¹ is —CH(CH₃)(CH₂)mCH₂N(CH₃)C(O)NH—. In some embodiments, L¹ is —CH(CH₃)(CH₂)mCH₂N(CH₃)C(NH)NH—. In some embodiments, L¹ is —CH(CH₃)(CH₂)mCH(CH₃)NHC(O)NH—. In some embodiments, L¹ is —CH(CH₃)(CH₂)mCH(CH₃)NHC(NH)NH—. In some embodiments, L¹ is —CH(CH₃)(CH₂)mCH(CH₃)N(CH₃)C(O)NH—. In some embodiments, L¹ is —CH(CH₃)(CH₂)mCH(CH₃)N(CH₃)C(NH)NH—. In some embodiments, L¹ is —CH(CH₃)(CH₂)mCH(CH₃)NHC(O)NHS(O)₂—. In some embodiments, L¹ is —CH(CH₃)(CH₂)mCH(CH₃)NHC(NH)NHS(O)₂—. In some embodiments, L¹ is —CH(CH₃)(CH₂)mCH(CH₃)N(CH₃)C(O)NHS(O)₂—. In some embodiments, L¹ is —CH(CH₃)(CH₂)mCH(CH₃)N(CH₃)C(NH)NHS(O)₂—. In some embodiments, L¹ is —CH(CH₃)(CH₂)mCH(CH₃)NHS(O)₂—. In some embodiments, L¹ is —CH(CH₃)(CH₂)mCH(CH₃)N(CH₃)S(O)₂—. In some embodiments, L¹ is —CH(CH₃)(CH₂)mCH₂NHC(S)NHS(O)₂—. In some embodiments, L¹ is —CH(CH₃)(CH₂)mCH₂N(CH₃)C(S)NHS(O)₂—. In some embodiments, L¹ is —CH(CH₃)(CH₂)mCH₂NHC(S)NH—. In some embodiments, L¹ is —CH(CH₃)(CH₂)mCH₂N(CH₃)C(S)NH—. In some embodiments, L¹ is —CH(CH₃)(CH₂)mCH(CH₃)NHC(S)NHS(O)₂—. In some embodiments, L¹ is —CH(CH₃)(CH₂)mCH(CH₃)N(CH₃)C(S)NHS(O)₂—. In some embodiments, L¹ is —CH(CH₃)(CH₂)mCH(CH₃)NHC(S)NH—. In some embodiments, L¹ is —CH(CH₃)(CH₂)mCH(CH₃)N(CH₃)C(S)NH—. In some embodiments, L¹ is —CH(CH₃)(CH₂)mCH₂NHC(O)C(O)NH—. In some embodiments, L¹ is —CH(CH₃)(CH₂)mCH₂N(CH₃)C(O)C(O)NH—. In some embodiments, —CH(CH₃)— is R. In some embodiments, —CH(CH₃)— is S. In some embodiments, when there are two —CH(CH₃)—, the one on the left is R, and the one on the right is S; in some embodiments, the one on the left is R, and the one on the right is R; in some embodiments, the one on the left is S, and the one on the right is R; in some embodiments, the one on the left is S, and the one on the right is S.

In some embodiments, L¹ is —CH(CH₃)—(CH₂)m-. In some embodiments, L¹ is —CH(CH₃)—(CH₂)m-CH₂—. In some embodiments, L¹ is —CH(CH₃)—(CH₂)m-CH₂NH—. In some embodiments, L¹ is —CH(CH₃)—(CH₂)m-CH₂NHC(O)—. In some embodiments, L¹ is —CH(CH₃)—(CH₂)m-CH₂NHC(O)NH—. In some embodiments, L¹ is —CH(CH₃)—(CH₂)m-CH₂NHC(O)NH—S(O)₂—. In some embodiments, L¹ is —CH(CH₃)—(CH₂)m-CH₂NH—S(O)₂—. In some embodiments, L¹ is —CH(CH₃)—(CH₂)m-CH₂N(CH₃)—. In some embodiments, L¹ is —CH(CH₃)—(CH₂)m-CH₂N(CH₃)C(O)—. In some embodiments, L¹ is —CH(CH₃)—(CH₂)m-CH₂N(CH₃)C(O)NH—. In some embodiments, L¹ is —CH(CH₃)—(CH₂)m-CH₂N(CH₃)—S(O)₂—. In some embodiments, L¹ is —CH(CH₃)—(CH₂)m-CH₂N(CH₃)C(O)NH—S(O)₂—. In some embodiments, L¹ is —CH(CH₃)—(CH₂)m-CH(CH₃)—. In some embodiments, L¹ is —CH(CH₃)—(CH₂)m-CH(CH₃)NH—. In some embodiments, L¹ is —CH(CH₃)—(CH₂)m-CH(CH₃)NHC(O)—. In some embodiments, L¹ is —CH(CH₃)—(CH₂)m-CH(CH₃)NHC(O)NH—. In some embodiments, L¹ is —CH(CH₃)—(CH₂)m-CH(CH₃)NHC(O)NH—S(O)₂—. In some embodiments, L¹ is —CH(CH₃)—(CH₂)m-CH(CH₃)NH—S(O)₂—. In some embodiments, L¹ is —CH(CH₃)—(CH₂)m-CH(CH₃)N(CH₃)—. In some embodiments, L¹ is —CH(CH₃)—(CH₂)m-CH(CH₃)N(CH₃)C(O)—. In some embodiments, L¹ is —CH(CH₃)—(CH₂)m-CH(CH₃)N(CH₃)C(O)NH—. In some embodiments, L¹ is —CH(CH₃)—(CH₂)m-CH(CH₃)N(CH₃)—S(O)₂—. In some embodiments, L¹ is —CH(CH₃)—(CH₂)m-CH(CH₃)N(CH₃)C(O)NH—S(O)₂—.

In some embodiments, L^{s0} is —CH(R′)— or substituted —CH₂—, wherein the carbon atom is chiral and is S. In some embodiments, L^{s0} is —CH(R′)— or substituted —CH₂—, wherein the carbon atom is chiral and is R. In some embodiments, L^{s1b} is —CH(R′)— or substituted —CH₂—, wherein the carbon atom is chiral and is S. In some embodiments, L^{s1b} is —CH(R′)— or substituted —CH₂—, wherein the carbon atom is chiral and is R. In some embodiments, each of L^{s0} and L^{s1b} is independently —CH(R′)— or substituted —CH₂— wherein the carbon atom is chiral, and the configurations of the two chiral carbon atoms (the first for L^{s0} and the second for L^{s1b}) is SS. In some embodiments, they are SR. In some embodiments, they are R^{s}. In some embodiments, they are RR.

In some embodiments, R^{L} is R^{s} as described herein. In some embodiments, R^{L} is R′. In some embodiments, R^{L} is —S(O)₂N(R^{s})₂. In some embodiments, R^{L} is —S(O)₂NHR^{s}. In some embodiments, R^{L} is —N(R^{s})₂. In some embodiments, R^{L} is —NHR^{s}. In some embodiments, R^{L} is —S(O)₂R^{s}. In some embodiments, R^{L} is —C(O)N(R′)S(O)₂R^{s}. In some embodiments, R^{L} is —C(O)NHS(O)₂R^{s}. In some embodiments, R^{L} is —C(S)N(R′)S(O)₂R^{s}. In some embodiments, R^{L} is —C(S)NHS(O)₂R^{s}. In some embodiments, R^{L} is —C(O)C(O)N(R′)S(O)₂R^{s}. In some embodiments, R^{L} is —C(O)C(O)NHS(O)₂R^{s}. In some embodiments, R^{L} is —C(O)N(R′)S(O)₂N(R^{s})₂. In some embodiments, R^{L} is —C(O)NHS(O)₂N(R^{s})₂. In some embodiments, R^{L} is —C(O)NHS(O)₂NHR^{s}. In some embodiments, R^{L} is C(O)N(R^{s})₂. In some embodiments, R^{L} is —C(O)N(R^{s})₂. In some embodiments, R^{L} is —C(O)NHR^{s}. In some embodiments, R^{L} is —N(R′)C(O)N(R′)S(O)₂R^{s}. In some embodiments, R^{L} is —NHC(O)NHS(O)₂R^{s}. In some embodiments, R^{L} is —N(R′)C(S)N(R′)S(O)₂R^{s}. In some embodiments, R^{L} is —NHC(S)NHS(O)₂R^{s}. In some embodiments, R^{L} is —N(R′)C(O)C(O)N(R′)S(O)₂R^{s}. In some embodiments, R^{L} is —NHC(O)C(O)NHS(O)₂R^{s}. In some embodiments, R^{L} is —N(R′)C(O)N(R′)S(O)₂N(R^{s})₂. In some embodiments, R^{L} is —NHC(O)NHS(O)₂N(R^{s})₂. In some embodiments, R^{L} is —NHC(O)NHS(O)₂NHR^{s}. In some embodiments, R^{L} is N(R′)C(O)N(R^{s})₂. In some embodiments, R^{L} is —NHC(O)N(R^{s})₂. In some embodiments, R^{L} is —NHC(O)NHR^{s}. In some embodiments, R^{L} is —S(O)₂N(R′)₂. In some embodiments, R^{L} is —S(O)₂NHR′. In some embodiments, R^{L} is —N(R′)₂. In some embodiments, R^{L} is —NHR. In some embodiments, R^{L} is —S(O)₂R′. In some embodiments, R^{L} is —C(O)N(R′)S(O)₂R′. In some embodiments, R^{L} is —C(O)NHS(O)₂R′. In some embodiments, R^{L} is —C(S)N(R′)S(O)₂R′. In some embodiments, R^{L} is —C(S)NHS(O)₂R′. In some embodiments, R^{L} is —C(O)C(O)N(R′)S(O)₂R′. In some embodiments, R^{L} is —C(O)C(O)NHS(O)₂R′. In some embodiments, R^{L} is —C(O)N(R′)S(O)₂N(R′)₂. In some embodiments, R^{L} is —C(O)NHS(O)₂N(R′)₂. In some embodiments, R^{L} is —C(O)NHS(O)₂NHR′. In some embodiments, R^{L} is —C(O)N(R′)₂. In some embodiments, R^{L} is —C(O)N(R′)₂. In some embodiments, R^{L} is —C(O)NHR′. In some embodiments, R^{L} is —N(R′)C(O)N(R′)S(O)₂R′. In some embodiments, R^{L} is —NHC(O)NHS(O)₂R′. In some embodiments, R^{L} is —N(R′)C(S)N(R′)S(O)₂R′. In some embodiments, R^{L} is —NHC(S)NHS(O)₂R′. In some embodiments, R^{L} is —N(R′)C(O)C(O)N(R′)S(O)₂R′. In some embodiments, R^{L} is —NHC(O)C(O)NHS(O)₂R′. In some embodiments, R^{L} is —N(R′)C(O)N(R′)S(O)₂N(R′)₂. In some embodiments, R^{L} is —NHC(O)NHS(O)₂N(R′)₂. In some embodiments, R^{L} is —NHC(O)NHS(O)₂NHR′. In some embodiments, R^{L} is —N(R′)C(O)N(R′)₂. In some embodiments, R^{L} is —NHC(O)N(R′)₂. In some embodiments, R^{L} is —NHC(O)NHR′.

In some embodiments, R^{L} is -L″-R′, wherein each variable is independently as described herein. In some embodiments, R^{s} is -L″-R′, wherein each variable is independently as described herein. In some embodiments, L″ is a covalent bond. In some embodiments, L″ is optionally substituted —CH₂—CH₂—. In some embodiments, L″ is —CH₂—CH₂—. In some embodiments, L″ is —CH₂-Cy-, wherein the —CH₂— is optionally substituted. In some embodiments, one or both methylene units of L″ is optionally substituted replaced with a moiety as described herein. In some embodiments, a methylene unit is replaced with -Cy-. In some embodiments, L″ is —CH₂-Cy-, wherein the —CH₂— is optionally substituted. In some embodiments, L″ is —C(R′)₂-Cy-. In some embodiments, —C(R′)₂— is —C(CH₃)₂—. In some embodiments, -Cy-is bonded to R′. In some embodiments, L″ is -Cy-. In some embodiments, L″ is -Cy-Cy-. In some embodiments, -Cy- is optionally substituted 3-10 membered cycloalkyl, e.g., in some embodiments, -Cy-is

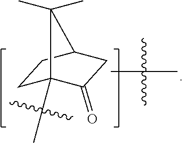

In some embodiments, -Cy- is an optionally substituted monocyclic 3-, 4-, 5-, 6-, or 7-memebered ring having 0-2 heteroatoms. In some embodiments, -Cy- is optionally substituted phenylene. In some embodiments, -Cy- is phenylene. In some embodiments, -Cy- is an optionally substituted bivalent heteroaryl ring having 1-4 heteroatoms. In some embodiments, -Cy- is an optionally substituted monocyclic 5- or 6-membered bivalent heteroaryl ring having 1-4 heteroatoms. In some embodiments, -Cy- is an optionally substituted bicyclic 9- or 10-membered bivalent heteroaryl ring having 1-4 heteroatoms. In some embodiments, —CH₂— is bonded to R′. In some embodiments, a methylene unit is replaced with optionally substituted —CH═CH—. In some embodiments, L″ is —CH═CH—. In some embodiments, L″ is —CH₂—N(R′)—, wherein the —CH₂— is optionally substituted. In some embodiments, L″ is —CH₂—NH—. In some embodiments, —N(R′)— is bonded to R′. In some embodiments, L″ is —N(R′)—. In some embodiments, L″ is —NH—. In some embodiments, L″ is —N(CH₃)—. In some embodiments, R′ is —COOH. In some embodiments, R′ is —C(O)NH₂. In some embodiments, R′ is —CH₂—CN, wherein —CH₂— is optionally substituted. In some embodiments, R′ is —C(R′)₂—CN. In some embodiments, R′ is —CH₂—OH, wherein the —CH₂— is optionally substituted. In some embodiments, R′ is —COOH. Certain embodiments of R^{L}, L″ and R′ are described in Table 1 as examples.

In some embodiments, a provided compound, e.g., a compound of formula I or a salt thereof, a compound of formula II or a salt thereof, etc., is a compound of formula II′ or a salt thereof:

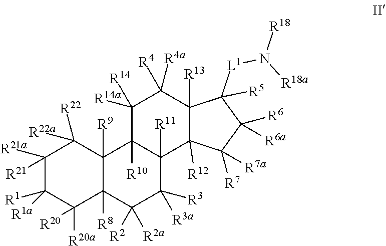

wherein:
   R¹⁸ is R′;
   R^{18a} is —C(O)N(R′)S(O)₂R^{L}, —C(S)N(R′)S(O)₂R^{L}, —C(O)C(O)N(R′)S(O)₂R^{L}, —S(O)₂R^{L}, —C(O)N(R′)R^{L}, or R^{L}; and
   each other variable is independently as described herein.

In some embodiments, a provided compound, e.g., a compound of formula I or a salt thereof, a compound of formula II or a salt thereof, etc., is a compound of formula II″ or a salt thereof:

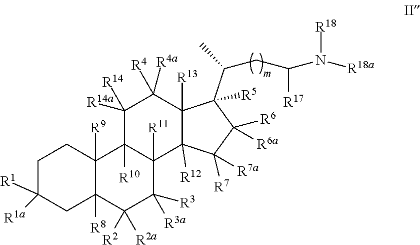

wherein:
   R¹⁷ is R′;
   R¹⁸ is R′; R^{18a} is —C(O)NHS(O)₂R^{L}, —C(S)NHS(O)₂R^{L}, —C(O)C(O)NHS(O)₂R^{L}, —S(O)₂R^{L}, —C(O)NHR^{L}, or R^{L}; and
   each other variable is independently as described herein.

In some embodiments, R^{18a} is R^{L}. In some embodiments, —N(R¹⁸)(R^{18a}) is R^{L}, wherein R^{L} is —N(R^{s})₂, —N(R′)C(O)N(R′)S(O)₂R′, —N(R′)C(S)N(R′)S(O)₂R′, —N(R′)C(O)C(O)N(R′)S(O)₂R′, —N(R′)C(O)N(R′)S(O)₂N(R^{s})₂, —N(R′)C(O)R^{s}, —N(R′)C(O)OR^{s}, —N(R′)C(O)N(R^{s})₂, or —N(R′)S(O)₂R^{s}. In some embodiments, R^{L} is —N(R^{s})₂, —N(R′)C(O)N(R′)S(O)₂R^{s}, —N(R′)C(S)N(R′)S(O)₂R^{s}, —N(R′)C(O)C(O)N(R′)S(O)₂R^{s}, —N(R′)C(O)N(R^{s})₂, or —N(R′)S(O)₂R^{s}. In some embodiments, R^{18a} is —C(O)NHS(O)₂R^{s}, —C(S)NHS(O)₂R^{s}, —C(O)C(O)NHS(O)₂R^{s}, —S(O)₂R^{s}, —C(O)NHR^{s}, or R^{s}. In some embodiments, —N(R¹⁸)(R^{18a}) is R^{L}, wherein R^{L} is —N(R′)₂, —N(R′)C(O)N(R′)S(O)₂R′, —N(R′)C(S)N(R′)S(O)₂R′, —N(R′)C(O)C(O)N(R′)S(O)₂R′, —N(R′)C(O)N(R′)S(O)₂N(R′)₂, —N(R′)C(O)R′, —N(R′)C(O)OR′, —N(R′)C(O)N(R′)₂, or —N(R′)S(O)₂R′. In some embodiments, R^{L} is —N(R′)₂, —N(R′)C(O)N(R′)S(O)₂R′, —N(R′)C(S)N(R′)S(O)₂R′, —N(R′)C(O)C(O)N(R′)S(O)₂R′, —N(R′)C(O)N(R′)₂, or —N(R′)S(O)₂R′. In some embodiments, R^{18a} is —C(O)NHS(O)₂R′, —C(S)NHS(O)₂R′, —C(O)C(O)NHS(O)₂R′, —S(O)₂R′, —C(O)NHR′, or R′.

In some embodiments, a provided compound is a compound of formula II′-A, II′—B, or II′—C, or a salt thereof:

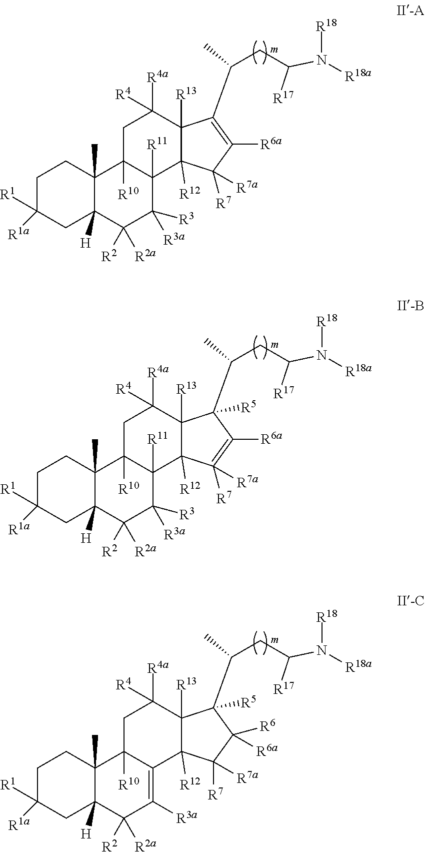

In some embodiments, a provided compound is a compound of a formula below or a salt thereof:

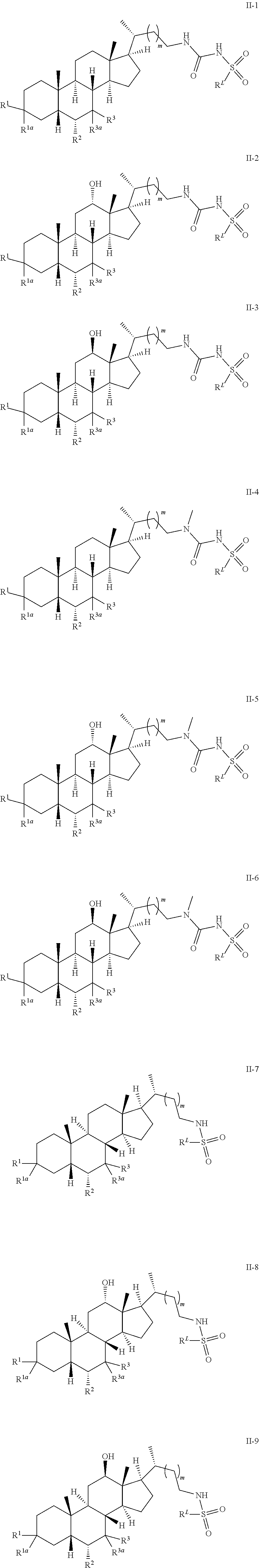

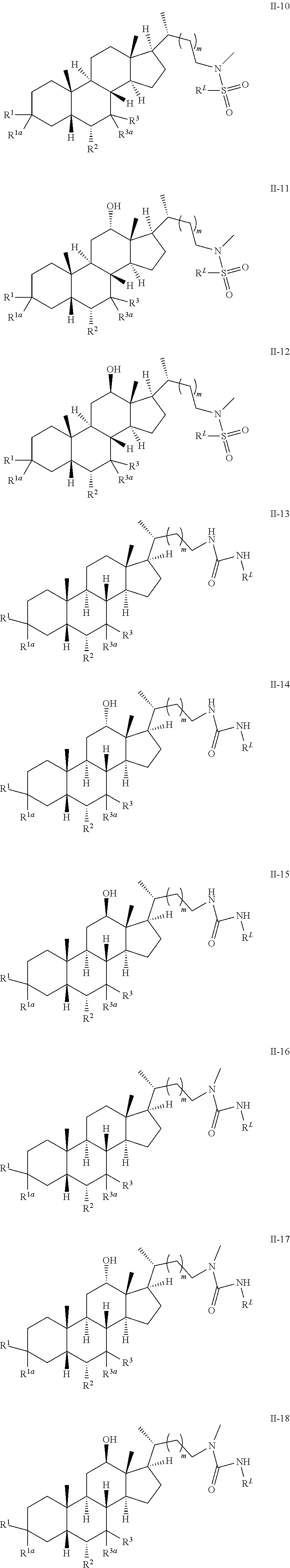

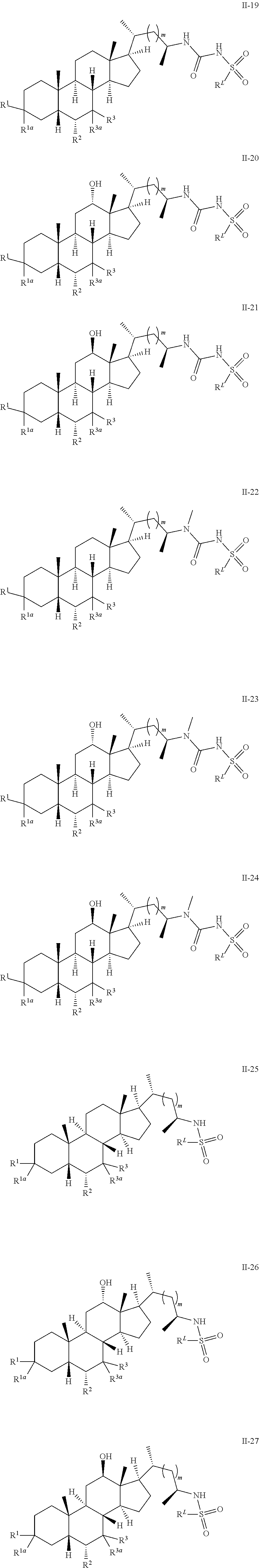

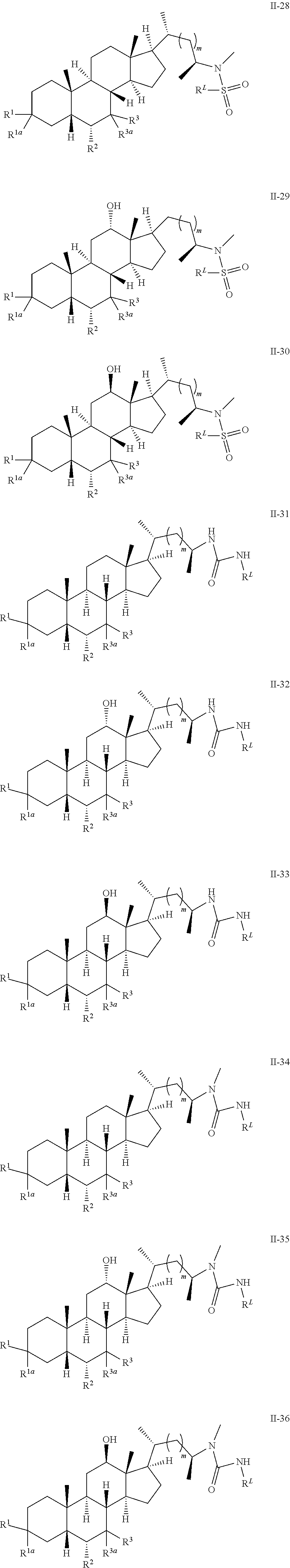

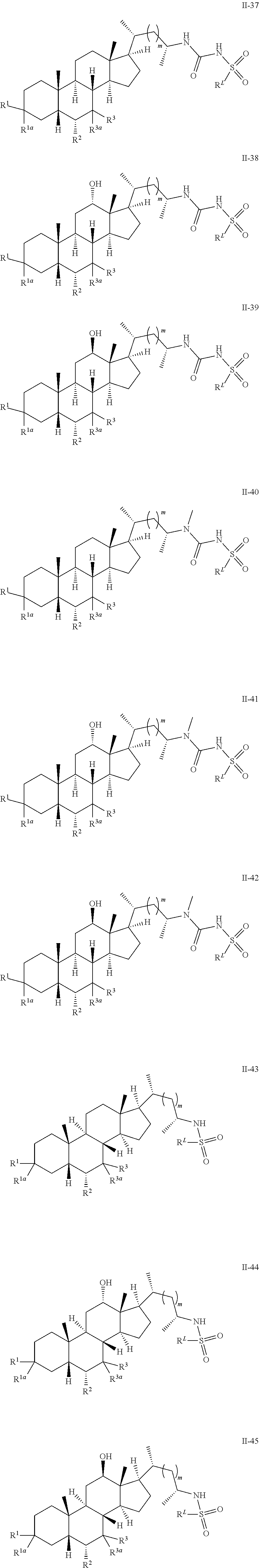

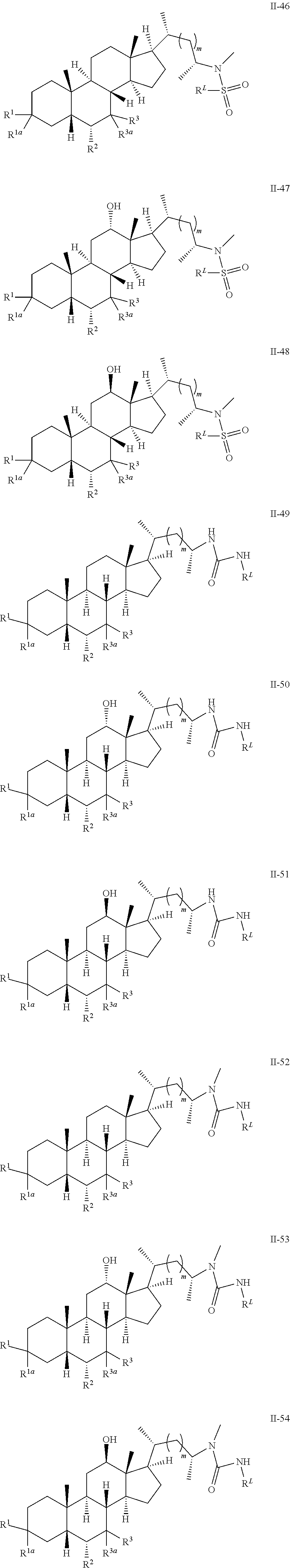

In some embodiments, a provided compound is a compound of a formula below or a salt thereof:

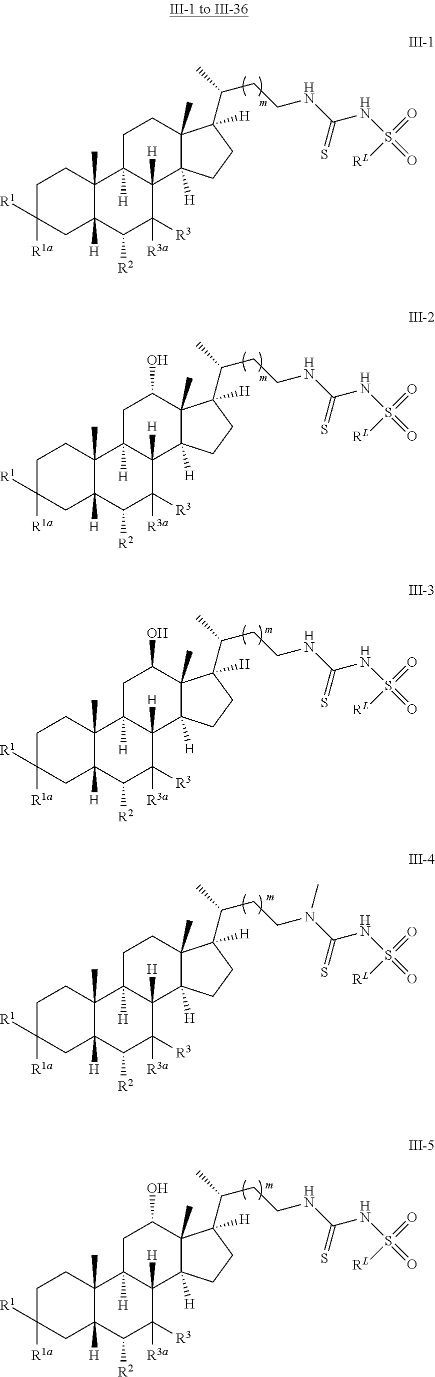

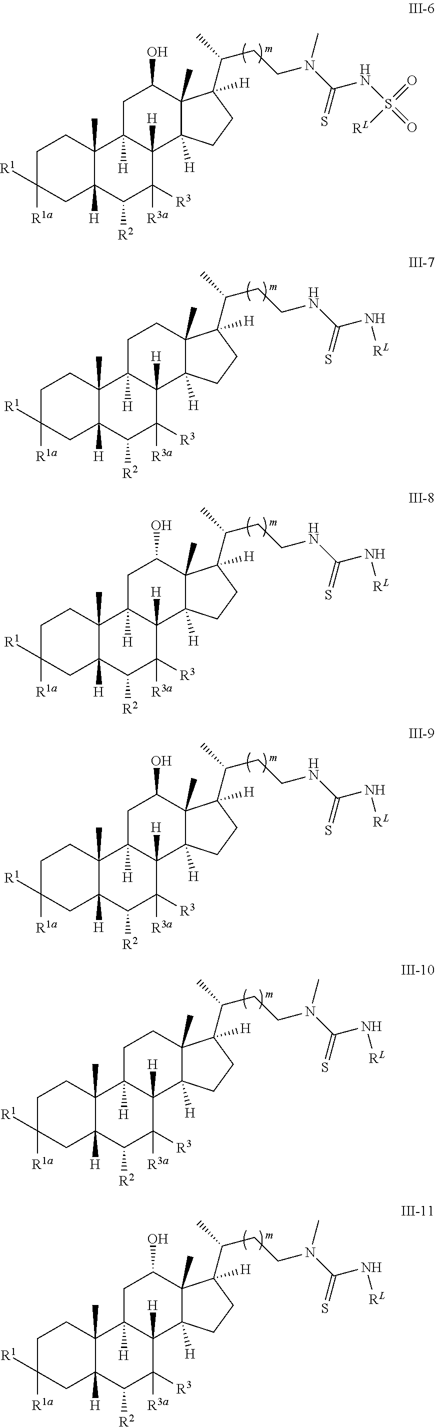

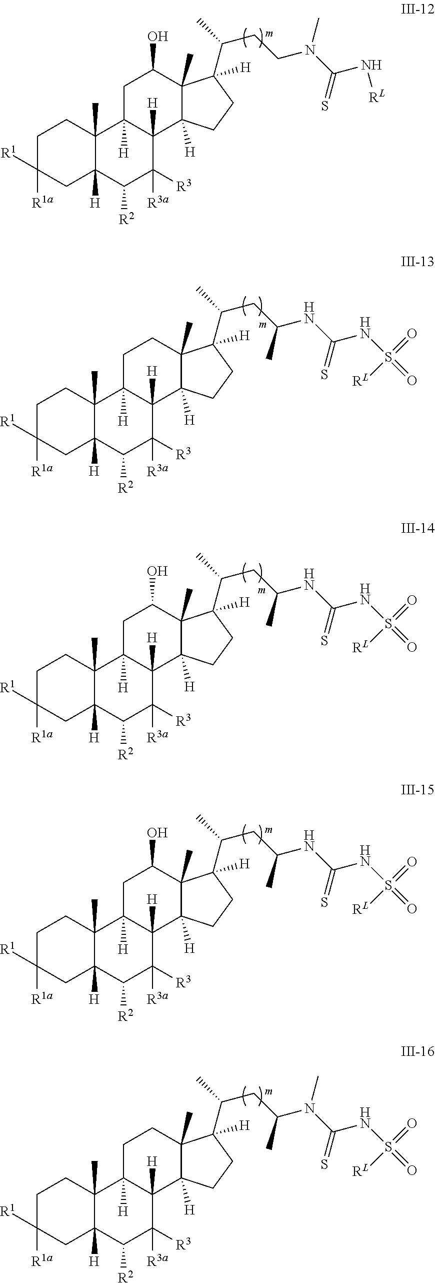

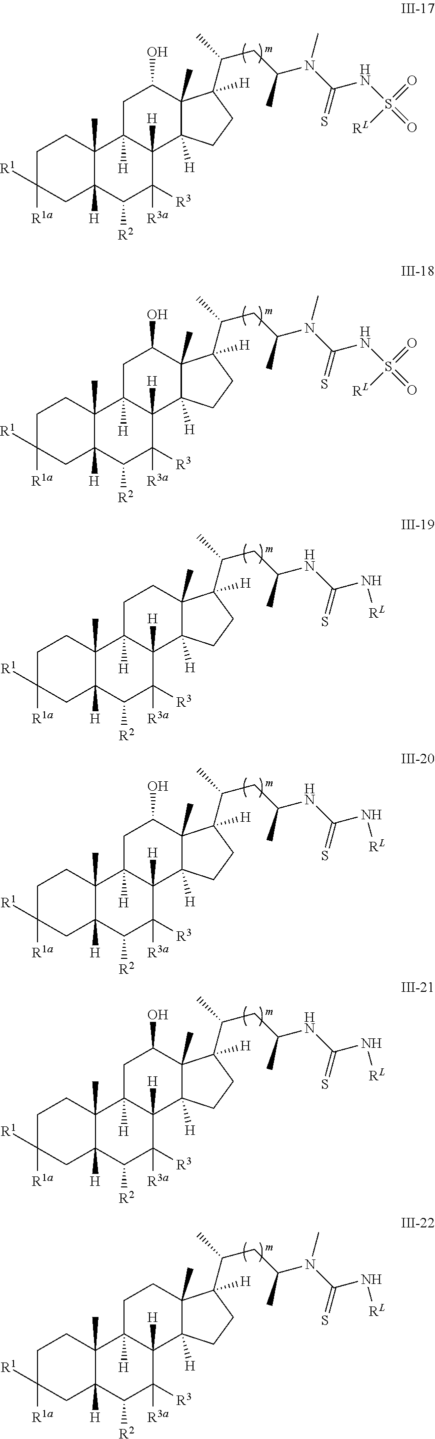

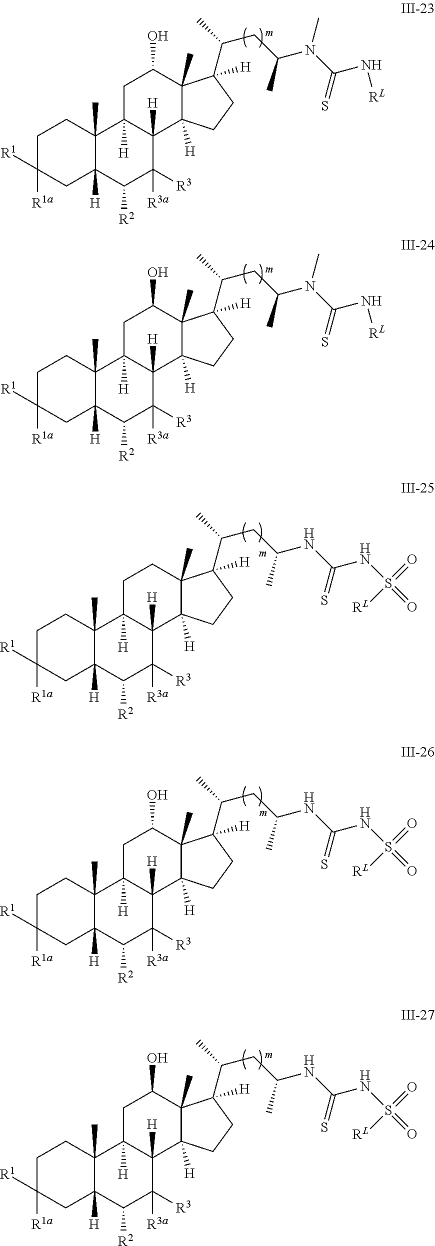

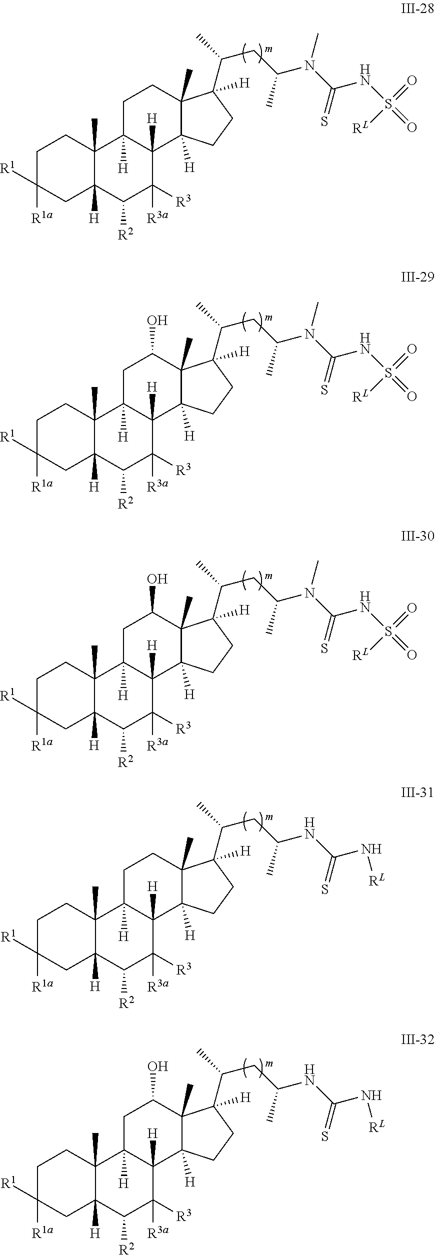

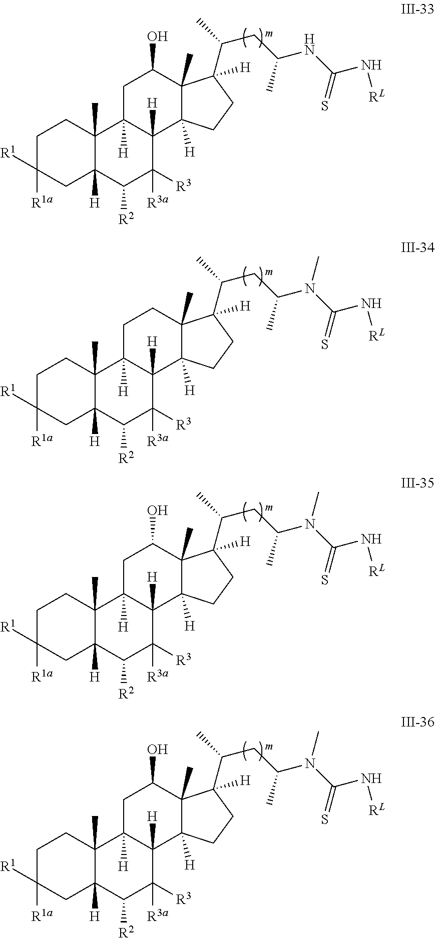

In some embodiments, a provided compound is a compound of a formula below or a salt thereof:

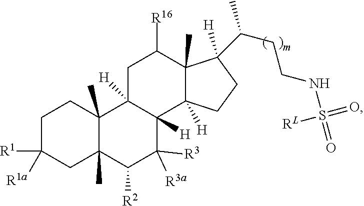

wherein each variable is independently as described herein. In some embodiments, a provided compound has the structure of

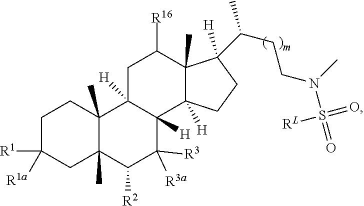

wherein each variable is independently as described herein. In some embodiments, a provided compound has the structure of

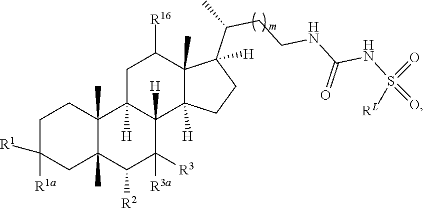

wherein each variable is independently as described herein. In some embodiments, a provided compound has the structure of

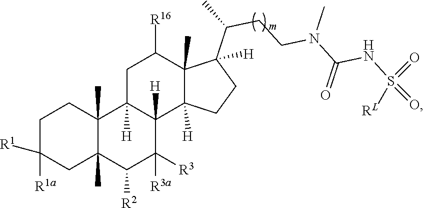

wherein each variable is independently as described herein. In some embodiments, a provided compound has the structure of

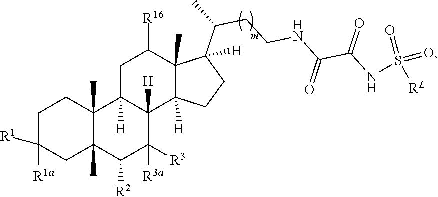

wherein each variable is independently as described herein.

In some embodiments, a provided compound has the structure of

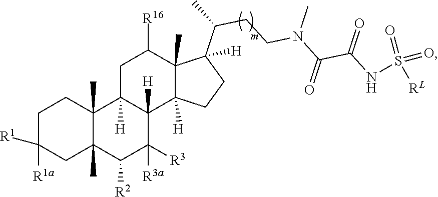

wherein each variable is independently as described herein.

In some embodiments, a provided compound has the structure of

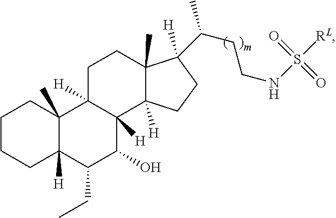

wherein each variable is independently as described herein. In some embodiments, a provided compound has the structure of

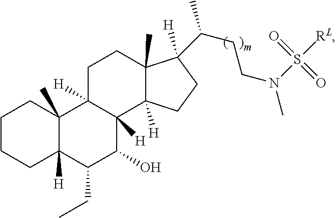

wherein each variable is independently as described herein. In some embodiments, a provided compound has the structure of

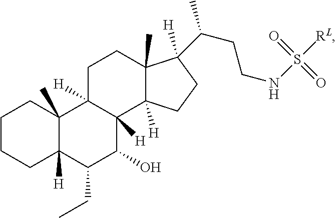

wherein each variable is independently as described herein. In some embodiments, a provided compound has the structure of

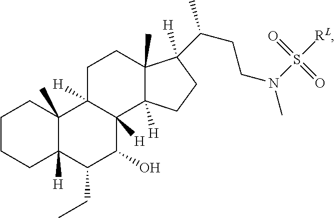

wherein each variable is independently as described herein. In some embodiments, a provided compound has the structure of

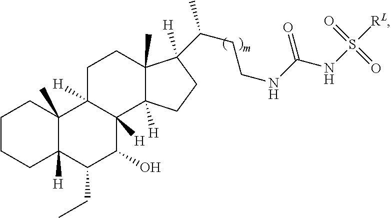

wherein each variable is independently as described herein. In some embodiments, a provided compound has the structure of

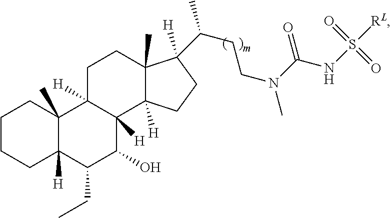

wherein each variable is independently as described herein. In some embodiments, a provided compound has the structure of

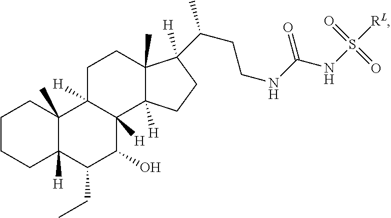

wherein each variable is independently as described herein. In some embodiments, a provided compound has the structure of

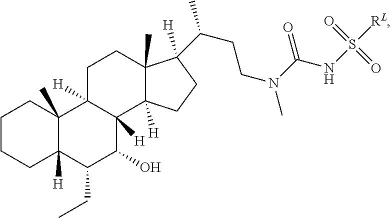

wherein each variable is independently as described herein. In some embodiments, a provided compound has the structure of

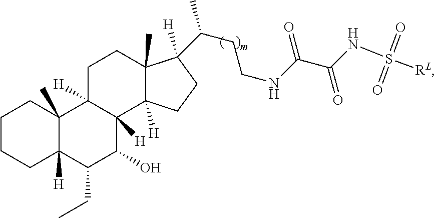

wherein each variable is independently as described herein. In some embodiments, a provided compound has the structure of

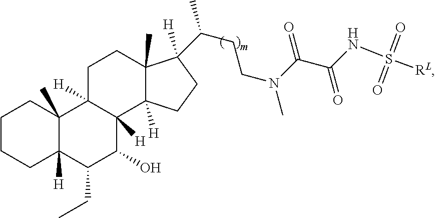

wherein each variable is independently as described herein. In some embodiments, a provided compound has the structure of

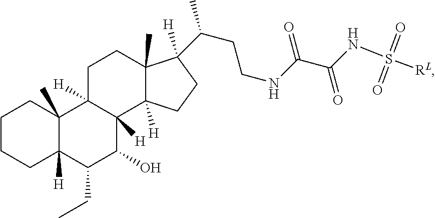

wherein each variable is independently as described herein. In some embodiments, a provided compound has the structure of

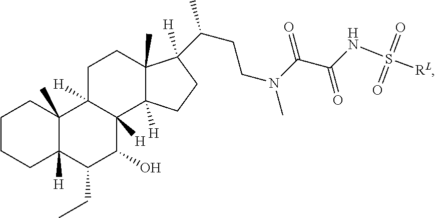

wherein each variable is independently as described herein. In some embodiments, each of R¹ and R^{1a} is —H, R² is optionally substituted C₁₋₆ aliphatic, one of R³ and R^{3a} is —H and the other is —OH, R¹⁶ is —H or —OH, and R^{L} is R as described herein. In some embodiments, each of R¹ and R^{1a} is —F, R² is optionally substituted C₁₋₆ aliphatic, one of R³ and R^{3a} is —H and the other is —OH, R¹⁶ is —H or —OH, and R^{L} is R as described herein. In some embodiments, one of R¹ and R^{1a} is —F and the other is —H, R² is optionally substituted C₁₋₆ aliphatic, one of R³ and R^{3a} is —H and the other is —OH, R¹⁶ is —H or —OH, and R^{L} is R as described herein. In some embodiments, R² is optionally substituted ethyl. In some embodiments, R² is ethyl. In some embodiments, R¹⁶ is —H. In some embodiments, m is 0. In some embodiments, m is 1. In some embodiments, m is 2. In some embodiments, R^{L} is R as described herein. In some embodiments, m and R^{L} are as described in any one of Table 1 to Table 7. In some embodiments, m and R^{L} are as described in Table 1. In some embodiments, m and R^{L} are as described in Table 2. In some embodiments, m and R^{L} are as described in Table 3. In some embodiments, m and R^{L}are as described in Table 4. In some embodiments, m and R^{L} are as described in Table 5. In some embodiments, m and R^{L} are as described in Table 6. In some embodiments, m and R^{L} are as described in Table 7.

In some embodiments, a provided compound is a compound of a formula below or a salt thereof:

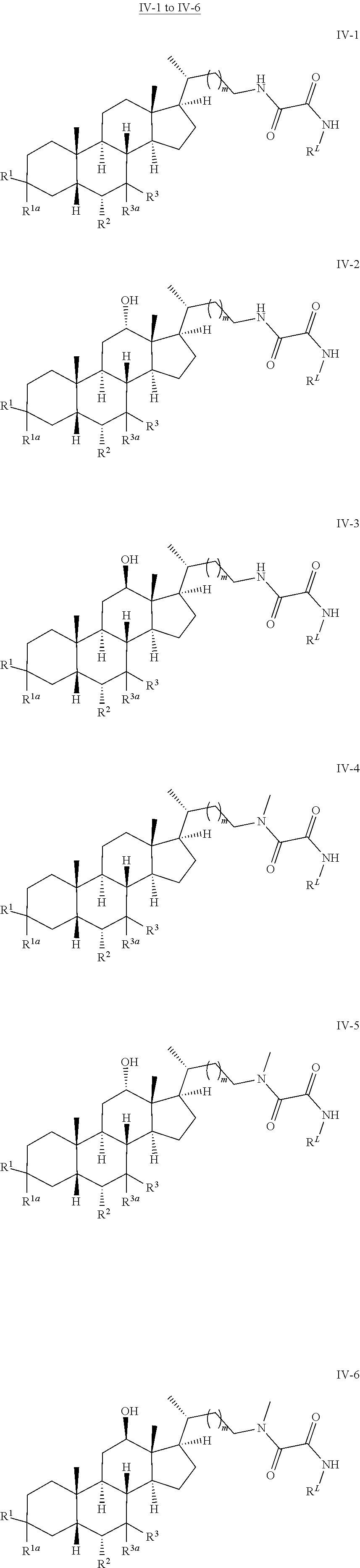

In some embodiments, m is 0, and R^{L} is R₁ as described herein. In some embodiments, m is 1, and R^{L} is R₁ as described herein. In some embodiments, m is 2, and R^{L} is R₁ as described herein. In some embodiments, m is 3, and R^{L} is R₁ as described herein. In some embodiments, R₁ is C₁₋₄ alkyl. In some embodiments, R₁ is C₁₋₄ haloalkyl. In some embodiments, R₁ is C₂₋₄ alkenyl. In some embodiments, R₁ is C₂₋₄ alkynyl. In some embodiments, R₁ is phenyl-C₁₋₄ alkyl. In some embodiments, R₁ is optionally substituted C₃₋₆ cycloalkyl. In some embodiments, R₁ is optionally substituted C₃₋₆ cycloalkyl-C₁₋₄ alkyl. In some embodiments, R₁ is 5- or 6-membered heterocyclyl. In some embodiments, R₁ is amino. In some embodiments, R₁ is optionally substituted phenyl. In some embodiments, R₁ is halogen. In some embodiments, R₁ is R as described herein. In some embodiments, R₁ is optionally substituted C₁₋₆ alkyl. In some embodiments, R₁ is methyl. In some embodiments, R₁ is ethyl. In some embodiments, R₁ is butyl. In some embodiments, R₁ is t-butyl. In some embodiments, R₁ is propyl. In some embodiments, R₁ is isopropyl. In some embodiments, R₁ is benzyl. In some embodiments, R₁ is vinyl. In some embodiments, R₁ is allyl. In some embodiments, R₁ is —CF₃. In some embodiments, R₁ is cyclopropyl. In some embodiments, R₁ is 1-methylcyclopropyl. In some embodiments, R₁ is cyclopropylmethyl. In some embodiments, R₁ is 1-pyrrolidinyl. In some embodiments, R₁ is 1-piperidinyl. In some embodiments, R₁ is 4-morpholinyl. In some embodiments, R₁ is —NH₂. In some embodiments, R₁ is dimethylamino. In some embodiments, R₁ is optionally substituted phenyl. In some embodiments, R₁ is phenyl. In some embodiments, R₁ is 4-trifluoromethoxyphenyl. In some embodiments, R₁ is 2-methylphenyl. In some embodiments, R₁ is 2-trifluoromethoxyphenyl. In some embodiments, R₁ is 2-fluorophenyl. In some embodiments, R₁ is 4-tert-butylphenyl. In some embodiments, R₁ is 2-naphthyl. In some embodiments, R₁ is 4-methylphenyl. In some embodiments, R₁ is 2-methoxyphenyl. In some embodiments, R₁ is —F. In some embodiments, R₁ is hexyl. In some embodiments, R₁ is 2-ethoxyethyl. In some embodiments, R₁ is

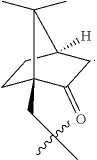 [SMILES: *C[C@]12CC[C@H](CC1=O)C2(C)C]

In some embodiments, R₁ is 2-methylpropyl. In some embodiments, R₁ is allyl. In some embodiments, R₁ is propargyl. In some embodiments, R₁ is 3-phenylallyl. In some embodiments, R₁ is cyclopentyl. In some embodiments, R₁ is

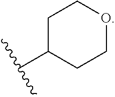 [SMILES: *C1CCOCC1]

In some embodiments, R₁ is

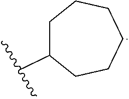 [SMILES: *C1CCCCCC1]

In some embodiments, R₁ is

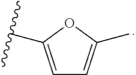 [SMILES: *c1ccc(C)o1]

In some embodiments, R₁ is

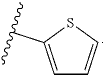 [SMILES: *c1cccs1]

In some embodiments, R₁ is

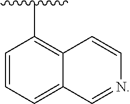 [SMILES: *c1cccc2cnccc12]

In some embodiments, R₁ is 3, 5-difluorophenyl. In some embodiments, R₁ is 2, 5-difluorophenyl. In some embodiments, R₁ is 2-trifluoromethyl-4-bromophenyl. In some embodiments, R₁ is

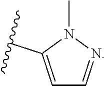 [SMILES: *c1ccnn1C]

In some embodiments, R₁ is

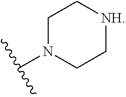 [SMILES: *N1CCNCC1]

In some embodiments, R₁ is

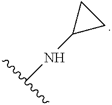 [SMILES: *NC1CC1]

In some embodiments, R₁ is

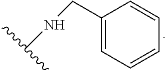 [SMILES: *NCc1ccccc1]

In some embodiments, R₁ is

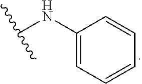 [SMILES: *Nc1ccccc1]

In some embodiments, R₁ is

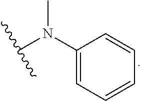 [SMILES: *N(C)c1ccccc1]

In some embodiments, R₁ is

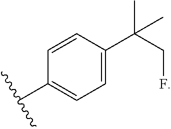 [SMILES: *c1ccc(C(C)(C)CF)cc1]

In some embodiments, R₁ is

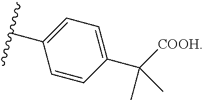 [SMILES: *c1ccc(C(C)(C)C(=O)O)cc1]

In some embodiments, R₁ is

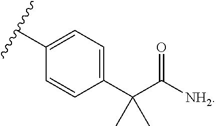 [SMILES: *c1ccc(C(C)(C)C(N)=O)cc1]

In some embodiments, R₁ is

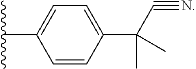 [SMILES: *c1ccc(C(C)(C)C#N)cc1]

In some embodiments, R₁ is

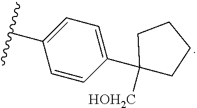 [SMILES: *c1ccc(C2(CO)CCCC2)cc1]

In some embodiments, R₁ is

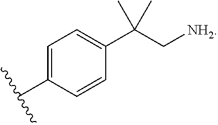 [SMILES: *c1ccc(C(C)(C)CN)cc1]

In some embodiments, R₁ is

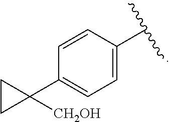 [SMILES: *c1ccc(C2(CO)CC2)cc1]

In some embodiments, R₁ is

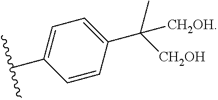 [SMILES: *c1ccc(C(C)(CO)CO)cc1]

In some embodiments, R₁ is

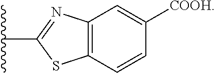 [SMILES: *c1nc2cc(C(=O)O)ccc2s1]

In some embodiments, R₁ is

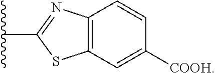 [SMILES: *c1nc2ccc(C(=O)O)cc2s1]

In some embodiments, R₁ is

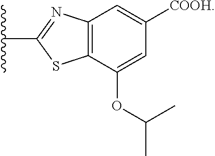 [SMILES: *c1nc2cc(C(=O)O)cc(OC(C)C)c2s1]

In some embodiments, certain combinations of m and R^{L} (or R₁) is described below. In some embodiments, m and R^{L} are as described in any one of Table 1 to Table 7. In some embodiments, m and R^{L} are as described in Table 1. In some embodiments, m and R^{L} are as described in Table 2. In some embodiments, m and R^{L} are as described in Table 3. In some embodiments, m and R^{L} are as described in Table 4. In some embodiments, m and R^{L} are as described in Table 5. In some embodiments, m and R^{L} are as described in Table 6. In some embodiments, m and R^{L} are as described in Table 7.

TABLE 1
Certain examples of m and R^{L}/R₁.

| Entry | m | R^{L}/R₁ |
|---|---|---|
| 1 | 0 | 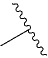 |
| 2 | 0 | 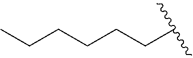 [SMILES: *CCCCCC] |
| 3 | 0 | 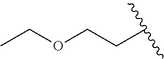 [SMILES: *CCOCC] |
| 4 | 0 | 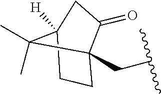 [SMILES: *C[C@]12CC[C@H](CC1=O)C2(C)C] |
| 5 | 0 | 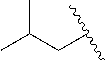 [SMILES: *CC(C)C] |
| 6 | 0 | 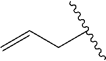 [SMILES: *CC=C] |
| 7 | 0 | 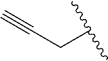 [SMILES: *CC#C] |
| 8 | 0 | 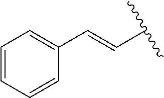 [SMILES: */C=C/c1ccccc1] |
| 9 | 0 | 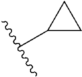 [SMILES: *C1CC1] |
| 10 | 0 | 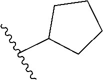 [SMILES: *C1CCCC1] |
| 11 | 0 | 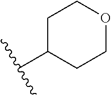 [SMILES: *C1CCOCC1] |
| 12 | 0 | 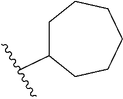 [SMILES: *C1CCCCCC1] |
| 13 | 0 | 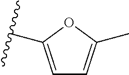 [SMILES: *c1ccc(C)o1] |
| 14 | 0 | 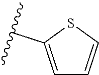 [SMILES: *c1cccs1] |
| 15 | 0 | 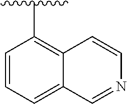 [SMILES: *c1cccc2cnccc12] |
| 16 | 0 | 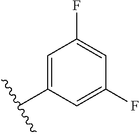 [SMILES: *c1cc(F)cc(F)c1] |
| 17 | | 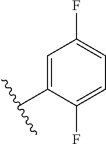 [SMILES: *c1cc(F)ccc1F] |
| 18 | | 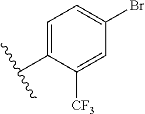 [SMILES: *c1ccc(Br)cc1C(F)(F)F] |
| 19 | 0 | 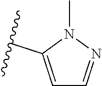 [SMILES: *c1ccnn1C] |
| 20 | 0 | 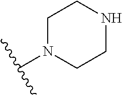 [SMILES: *N1CCNCC1] |
| 21 | 0 | 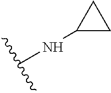 [SMILES: *NC1CC1] |
| 22 | 0 | 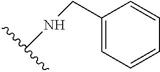 [SMILES: *NCc1ccccc1] |
| 23 | 0 | 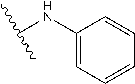 [SMILES: *Nc1ccccc1] |
| 24 | 0 | 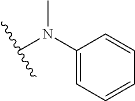 [SMILES: *N(C)c1ccccc1] |
| 25 | 0 | 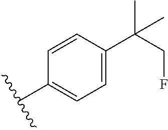 [SMILES: *c1ccc(C(C)(C)CF)cc1] |
| 26 | 0 | 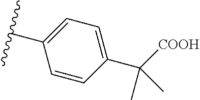 [SMILES: *c1ccc(C(C)(C)C(=O)O)cc1] |
| 27 | 0 | 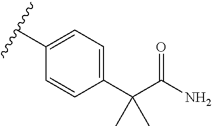 [SMILES: *c1ccc(C(C)(C)C(N)=O)cc1] |
| 28 | 0 | 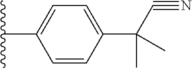 [SMILES: *c1ccc(C(C)(C)C#N)cc1] |
| 29 | 0 | 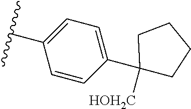 [SMILES: *c1ccc(C2(CO)CCCC2)cc1] |
| 30 | 0 | 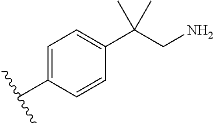 [SMILES: *c1ccc(C(C)(C)CN)cc1] |
| 31 | 0 | 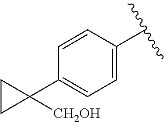 [SMILES: *c1ccc(C2(CO)CC2)cc1] |
| 32 | 0 | 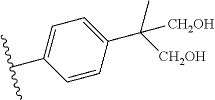 [SMILES: *c1ccc(C(C)(CO)CO)cc1] |
| 33 | 0 | 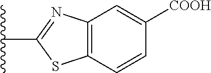 [SMILES: *c1nc2cc(C(=O)O)ccc2s1] |
| 34 | 0 | 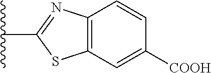 [SMILES: *c1nc2ccc(C(=O)O)cc2s1] |
| 35 | 0 | 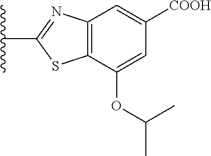 [SMILES: *c1nc2cc(C(=O)O)cc(OC(C)C)c2s1] |
| 36 | 1 | 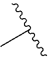 |
| 37 | 1 | 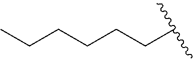 [SMILES: *CCCCCC] |
| 38 | 1 | 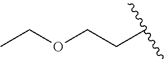 [SMILES: *CCOCC] |
| 39 | 1 | 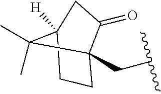 [SMILES: *C[C@]12CC[C@H](CC1=O)C2(C)C] |
| 40 | 1 | 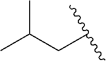 [SMILES: *CC(C)C] |
| 41 | 1 | 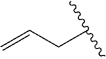 [SMILES: *CC=C] |
| 42 | 1 | 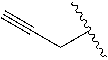 [SMILES: *CC#C] |
| 43 | 1 | 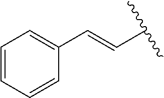 [SMILES: */C=C/c1ccccc1] |
| 44 | 1 | 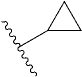 [SMILES: *C1CC1] |
| 45 | 1 | 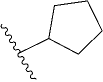 [SMILES: *C1CCCC1] |
| 46 | 1 | 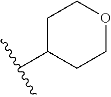 [SMILES: *C1CCOCC1] |
| 47 | 1 | 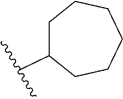 [SMILES: *C1CCCCCC1] |
| 48 | 1 | 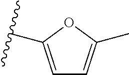 [SMILES: *c1ccc(C)o1] |
| 49 | 1 | 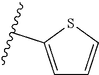 [SMILES: *c1cccs1] |
| 50 | 1 | 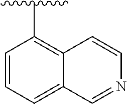 [SMILES: *c1cccc2cnccc12] |
| 51 | 1 | 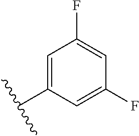 [SMILES: *c1cc(F)cc(F)c1] |
| 52 | | 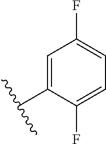 [SMILES: *c1cc(F)ccc1F] |
| 53 | | 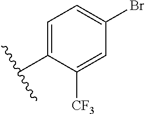 [SMILES: *c1ccc(Br)cc1C(F)(F)F] |
| 54 | 1 | 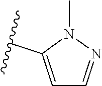 [SMILES: *c1ccnn1C] |
| 55 | 1 | 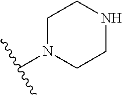 [SMILES: *N1CCNCC1] |
| 56 | 1 | 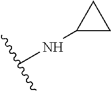 [SMILES: *NC1CC1] |
| 57 | 1 | 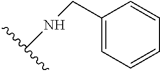 [SMILES: *NCc1ccccc1] |
| 58 | 1 | 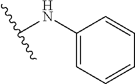 [SMILES: *Nc1ccccc1] |
| 59 | 1 | 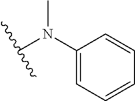 [SMILES: *N(C)c1ccccc1] |
| 60 | 1 | 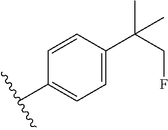 [SMILES: *c1ccc(C(C)(C)CF)cc1] |
| 61 | 1 | 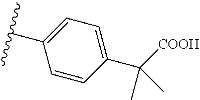 [SMILES: *c1ccc(C(C)(C)C(=O)O)cc1] |
| 62 | 1 | 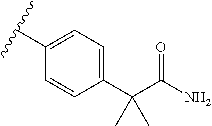 [SMILES: *c1ccc(C(C)(C)C(N)=O)cc1] |
| 63 | 1 | 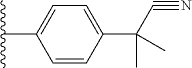 [SMILES: *c1ccc(C(C)(C)C#N)cc1] |
| 64 | 1 | 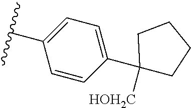 [SMILES: *c1ccc(C2(CO)CCCC2)cc1] |
| 65 | 1 | 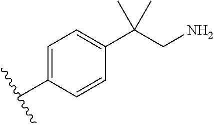 [SMILES: *c1ccc(C(C)(C)CN)cc1] |
| 66 | 1 | 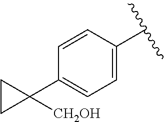 [SMILES: *c1ccc(C2(CO)CC2)cc1] |
| 67 | 1 | 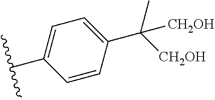 [SMILES: *c1ccc(C(C)(CO)CO)cc1] |
| 68 | 1 | 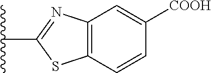 [SMILES: *c1nc2cc(C(=O)O)ccc2s1] |
| 69 | 1 | 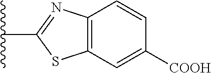 [SMILES: *c1nc2ccc(C(=O)O)cc2s1] |
| 70 | 1 | 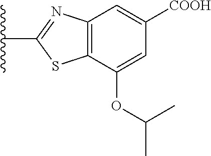 [SMILES: *c1nc2cc(C(=O)O)cc(OC(C)C)c2s1] |
| 71 | 2 | 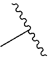 |
| 72 | 2 | 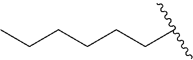 [SMILES: *CCCCCC] |
| 73 | 2 | 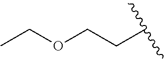 [SMILES: *CCOCC] |
| 74 | 2 | 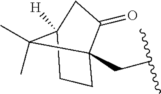 [SMILES: *C[C@]12CC[C@H](CC1=O)C2(C)C] |
| 75 | 2 | 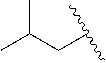 [SMILES: *CC(C)C] |
| 76 | 2 | 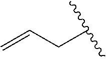 [SMILES: *CC=C] |
| 77 | 2 | 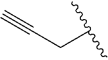 [SMILES: *CC#C] |
| 78 | 2 | 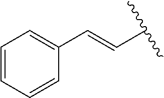 [SMILES: */C=C/c1ccccc1] |
| 79 | 2 | 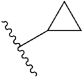 [SMILES: *C1CC1] |
| 80 | 2 | 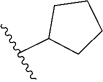 [SMILES: *C1CCCC1] |
| 81 | 2 | 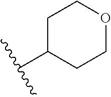 [SMILES: *C1CCOCC1] |
| 82 | 2 | 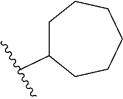 [SMILES: *C1CCCCCC1] |
| 83 | 2 | 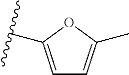 [SMILES: *c1ccc(C)o1] |
| 84 | 2 | 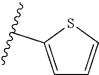 [SMILES: *c1cccs1] |
| 85 | 2 | 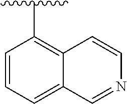 [SMILES: *c1cccc2cnccc12] |
| 86 | 2 | 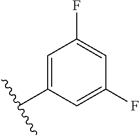 [SMILES: *c1cc(F)cc(F)c1] |
| 87 | | 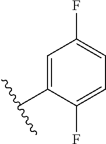 [SMILES: *c1cc(F)ccc1F] |
| 88 | | 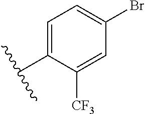 [SMILES: *c1ccc(Br)cc1C(F)(F)F] |
| 89 | 2 | 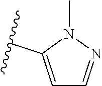 [SMILES: *c1ccnn1C] |
| 90 | 2 | 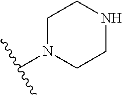 [SMILES: *N1CCNCC1] |
| 91 | 2 | 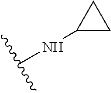 [SMILES: *NC1CC1] |
| 92 | 2 | 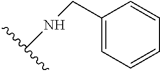 [SMILES: *NCc1ccccc1] |
| 93 | 2 | 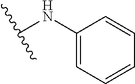 [SMILES: *Nc1ccccc1] |
| 94 | 2 | 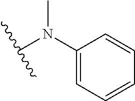 [SMILES: *N(C)c1ccccc1] |
| 95 | 2 | 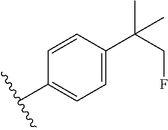 [SMILES: *c1ccc(C(C)(C)CF)cc1] |
| 96 | 2 | 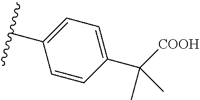 [SMILES: *c1ccc(C(C)(C)C(=O)O)cc1] |
| 97 | 2 | 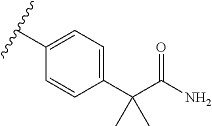 [SMILES: *c1ccc(C(C)(C)C(N)=O)cc1] |
| 98 | 2 | 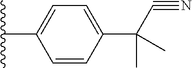 [SMILES: *c1ccc(C(C)(C)C#N)cc1] |
| 99 | 2 | 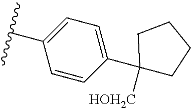 [SMILES: *c1ccc(C2(CO)CCCC2)cc1] |
| 100 | 2 | 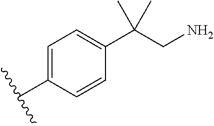 [SMILES: *c1ccc(C(C)(C)CN)cc1] |
| 101 | 2 | 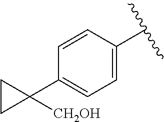 [SMILES: *c1ccc(C2(CO)CC2)cc1] |
| 102 | 2 | 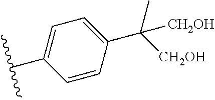 [SMILES: *c1ccc(C(C)(CO)CO)cc1] |
| 103 | 2 | 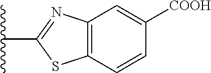 [SMILES: *c1nc2cc(C(=O)O)ccc2s1] |
| 104 | 2 | 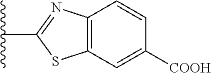 [SMILES: *c1nc2ccc(C(=O)O)cc2s1] |
| 105 | 2 | 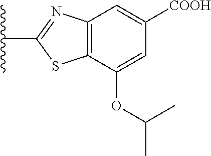 [SMILES: *c1nc2cc(C(=O)O)cc(OC(C)C)c2s1] |

In some embodiments, a compound is selected from II-1-1 to II-1-105, II-2-1 to II-2-105, II-3-1 to II-3-105, II-4-1 to II-4-105, II-5-1 to II-5-105, II-6-1 to II-6-105, II-7-1 to II-7-105, II-8-1 to II-8-105, II-9-1 to II-9-105, II-10-1 to II-10-105, II-11-1 to II-11-105, II-12-1 to II-12-105, II-13-1 to II-13-105, II-14-1 to II-14-105, II-15-1 to II-15-105, II-16-1 to II-16-105, II-17-1 to II-17-105, II-18-1 to II-18-105, II-19-1 to II-19-105, II-20-1 to II-20-105, II-21-1 to II-21-105, II-22-1 to II-22-105, II-23-1 to II-23-105, II-24-1 to II-24-105, II-25-1 to II-25-105, II-26-1 to II-26-105, II-27-1 to II-27-105, II-28-1 to II-28-105, II-29-1 to II-29-105, II-30-1 to II-30-105, II-31-1 to II-31-105, II-32-1 to II-32-105, II-33-1 to II-33-105, II-34-1 to II-34-105, II-35-1 to II-35-105, II-36-1 to II-36-105, II-37-1 to II-37-105, II-38-1 to II-38-105, II-39-1 to II-39-105, II-40-1 to II-40-105, II-41-1 to II-41-105, II-42-1 to II-42-105, II-43-1 to II-43-105, II-44-1 to II-44-105, II-45-1 to II-45-105, II-46-1 to II-46-105, II-47-1 to II-47-105, II-48-1 to II-48-105, II-49-1 to II-49-105, II-50-1 to II-50-105, II-51-1 to II-51-105, II-52-1 to II-52-105, II-53-1 to II-53-105, and II-54-1 to II-54-105, or a salt thereof, wherein a compound II-z1-z2 (wherein z1 is 1-54 and z2 is 1 to 105) has the structure of formula II-z1 (e.g., compound II-1-1 has the structure of formula II-1) wherein each of R¹, R^{1a}, R², R³ and R^{3a} is independently —H, and m and R^{L} is entry z2 as described in Table 1 (e.g., for compound II-1-1, its m and R^{L} is entry 1 in Table 1 (m is 0 and R^{L} is methyl).

In some embodiments, a compound is selected from III-1-1 to III-1-105, III-2-1 to III-2-105, III-3-1 to III-3-105, III-4-1 to III-4-105, III-5-1 to III-5-105, III-6-1 to III-6-105, III-7-1 to III-7-105, III-8-1 to III-8-105, III-9-1 to III-9-105, III-10-1 to III-10-105, III-11-1 to III-11-105, III-12-1 to III-12-105, III-13-1 to III-13-105, III-14-1 to III-14-105, III-15-1 to III-15-105, III-16-1 to III-16-105, III-17-1 to III-17-105, III-18-1 to III-18-105, III-19-1 to III-19-105, III-20-1 to III-20-105, III-21-1 to III-21-105, III-22-1 to III-22-105, III-23-1 to III-23-105, III-24-1 to III-24-105, III-25-1 to III-25-105, III-26-1 to III-26-105, III-27-1 to III-27-105, III-28-1 to III-28-105, III-29-1 to III-29-105, III-30-1 to III-30-105, III-31-1 to III-31-105, III-32-1 to III-32-105, III-33-1 to III-33-105, III-34-1 to III-34-105, III-35-1 to III-35-105, and III-36-1 to III-36-105, or a salt thereof, wherein a compound III-z1-z2 (wherein z1 is 1-36 and z2 is i to 105) has the structure of formula III-z1 (e.g., compound III-1-1 has the structure of formula III-1) wherein each of R¹, R^{1a}, R², R³ and R^{3a} is independently —H, and m and R^{L} is entry z2 as described in Table 1 (e.g., for compound III-1-1, its m and R^{L} is entry 1 in Table 1 (m is 0 and R^{L} is methyl)).

In some embodiments, a compound is selected from IV-1-1 to IV-1-105, IV-2-1 to IV-2-105, IV-3-1 to IV-3-105, IV-4-1 to IV-4-105, IV-5-1 to IV-5-105, and IV-6-1 to IV-6-105, or a salt thereof, wherein a compound IV-z1-z2 (wherein z1 is 1-6 and z2 is 1 to 105) has the structure of formula IV-z1 (e.g., compound IV-1-1 has the structure of formula IV-1) wherein each of R, R^{1a}, R², R³ and R^{3a} is independently —H, and m and R^{L} is entry z2 as described in Table 1 (e.g., for compound IV-1-1, its m and R^{L} is entry 1 in Table 1 (m is 0 and R^{L} is methyl)).

In some embodiments, each of R¹ and R^{1a} is independently selected from —H, halogen, —OH, —N(R′)₂, —C(O)R′, —SO₃R′, —OR′, and protected hydroxyl, or R¹ and R^{1a} are taken together to form ═O or =NR^{x} (e.g., in some embodiments, R¹ and R^{1a} are independently selected from —H and halogen; in some embodiments, R¹ and R^{1a} are —H; etc.);
each of R² and R^{2a} is independently selected from —H, halogen, or an optionally substituted group selected from C₁₋₈ alkyl, C₂₋₈ alkenyl, C₂₋₈ alkynyl, and C₃₋₈ cycloalkyl, or R² and R^{2a} are taken together to form ═O or ═NR^{x} (e.g., in some embodiments, one of R² and R^{2a} is C₁₋₄ alkyl and the other is —H; in some embodiments, one of R² and R^{2a} is ethyl and the other is —H; etc.);
each of R³ and R^{3a} is independently —H, halogen, —OH, —N(R′)₂, —C(O)R′, —SO₃R′, —OR′, protected hydroxyl, or an optionally substituted group selected from C₁₋₈ alkyl, C₂₋₈ alkenyl, C₂₋₈ alkynyl, and C₃₋₈ cycloalkyl, or R³ and R^{3a} are taken together to form ═O or ═NR^{x} (e.g., in some embodiments, R³ and R^{3a} are —H; in some embodiments, one of R³ and R^{3a} is —OH and the other is —H; etc.);
each of R⁴ and R^{4a} is independently —H, halogen, —CN, —N₃, —OH, —OS(O)₂R′, —SO₃R′, —C(O)R′, —OR′, —OP(O)(OR′)₂, —SR′, —N(R′)₂, or an optionally substituted group selected from C₁₋₈ alkyl, C₂₋₈ alkenyl and C₂₋₈ alkynyl, or R⁴ and R^{4a} are taken together to form ═O or ═NR^{x} (e.g., in some embodiments, R⁴ and R^{4a} are —H; in some embodiments, one of R⁴ and R^{4a} is —OH and the other is —H; in some embodiments, one of R² and R^{2a} is methyl and the other is —H; etc.);
R⁵ is —H, halogen or —OH;
each of R⁶ and R^{6a} is independently selected from —H, halogen, —CN, —N₃, —OH, —OS(O)₂R′, —SO₃R′, —C(O)R′, —OR′, —OP(O)(OR′)₂, —SR′, —N(R′)₂, or an optionally substituted group selected from C₁₋₈ alkyl, C₂₋₈ alkenyl and C₂₋₈ alkynyl, or R⁶ and R^{6a} are taken together to form ═O or ═NR^{x} (e.g., in some embodiments, R⁶ and R^{6a} are H); or R⁵ and one of R⁶ and R^{6a} are taken together with the carbon atoms to which they are attached to form optionally substituted —CH═CH— or an optionally substituted cycloalkyl (e.g., 3-10 membered) ring or heterocyclyl ring (e.g., 3-10 membered having 1-5 heteroatoms) (e.g., cyclopropyl, epoxide, etc.);
each of R⁷ and R^{7a} is independently selected from —H, halogen, —CN, —N₃, —OH, —OS(O)₂R′, —SO₃R′, —C(O)R′, —OR′, —OP(O)(OR′)₂, —SR′, —N(R′)₂, or an optionally substituted group selected from C₁₋₈ alkyl, C₂₋₈ alkenyl and C₂₋₈ alkynyl, or R⁷ and R^{7a} are taken together to form ═O or ═NR^{x} (e.g., in some embodiments, R⁷ and R^{7a} are H); or one of R⁶ and R^{6a} and one of R⁷ and R^{1a} are taken together with the carbon atoms to which they are attached to form optionally substituted —CH═CH— or an optionally substituted cycloalkyl (e.g., 3-10 membered) ring or heterocyclyl ring (e.g., 3-10 membered having 1-5 heteroatoms) (e.g., cyclopropyl, epoxide, etc.);
R¹⁰ is —H;
R¹¹ is —H, or R¹¹ and one of R³ and R^{3a} are taken together with the carbon atoms to which they are attached to form optionally substituted —CH═CH— or an optionally substituted cycloalkyl (e.g., 3-10 membered) ring or heterocyclyl ring (e.g., 3-10 membered having 1-5 heteroatoms) (e.g., cyclopropyl, epoxide, etc.);
R¹² is —H;
R¹³ is methyl;
R¹⁷ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl) (e.g., in some embodiments, R¹⁷ is C₁₋₄ alkyl; in some embodiments, R¹⁷ is methyl; etc.);
m is 0, 1, 2, or 3 (e.g., in some embodiments, m is 0; in some embodiments, m is 1; in some embodiments, m is 2; etc.);
R¹⁸ is —H or optionally substituted C₁₋₈ alkyl (e.g., in some embodiments, R¹⁸ is —H; in some embodiments, R¹⁸ is methyl; etc.);
R^{18a} is —C(O)NHS(O)₂R^{L}, —C(S)NHS(O)₂R^{L}, —C(O)C(O)NHS(O)₂R^{L}, —S(O)₂R^{L}, —C(O)NHR^{L} or R^{L} (e.g., in some embodiments, R^{L} is —H, halogen, —NR¹⁹R^{19a}, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl); in some embodiments, R^{L} is R₁ as described herein);
each of R¹⁹ and R^{19a} is independently R′ (e.g., in some embodiments, —H or an optionally substituted group selected from C₁₋₈ alkyl, C₂₋₈ alkenyl, C₂₋₈ alkynyl, C₃₋₈ cycloalkyl and heterocyclyl (e.g., 3-15 membered heterocyclyl having 1-5 heteroatoms; in some embodiments, R¹⁹ is Rₐ and R^{19a} is R_{b}; in some embodiments, R¹⁹ is —H; in some embodiments, R^{19a} is —H; etc.), or R¹⁹ and R^{19a} are taken together with the nitrogen atom to which they are attached to form an optionally substituted heterocyclic ring (e.g., an optionally substituted 3-15 membered heterocyclyl ring having 1-5 heteroatoms).

In some embodiments, a provided compound is a compound of a formula selected from below or a salt thereof, wherein each variable is independently as described herein:

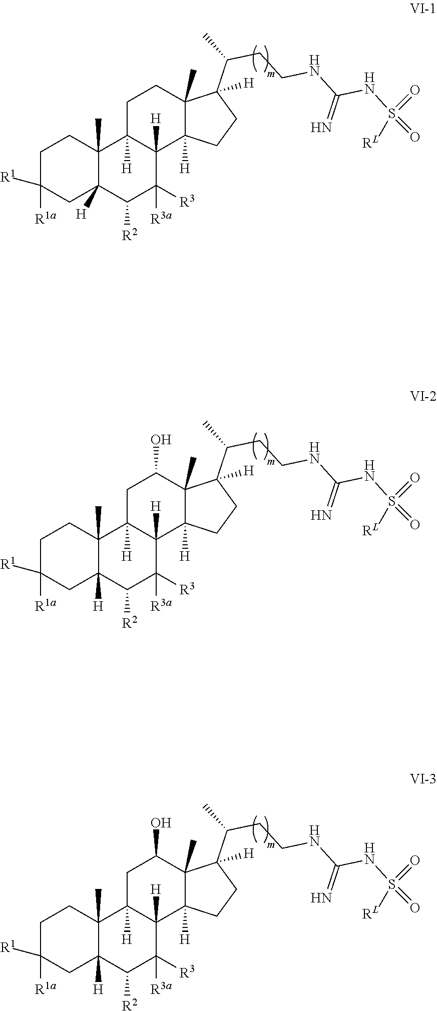

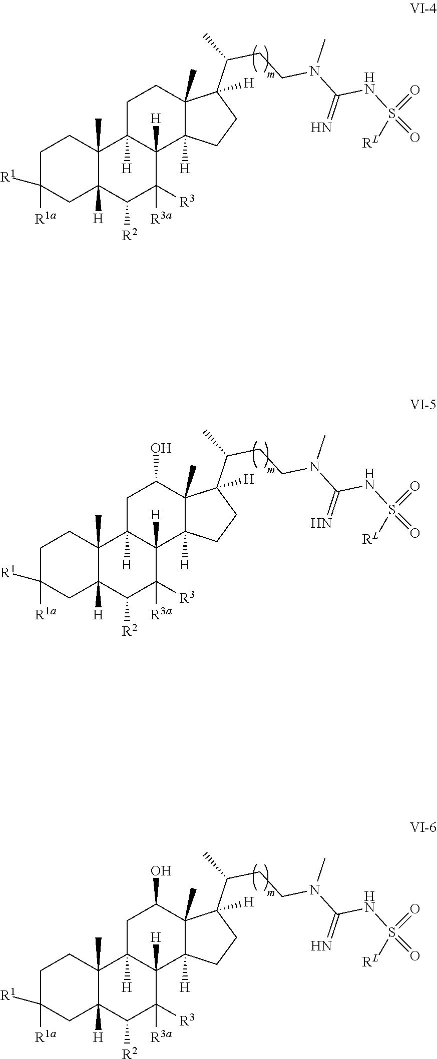

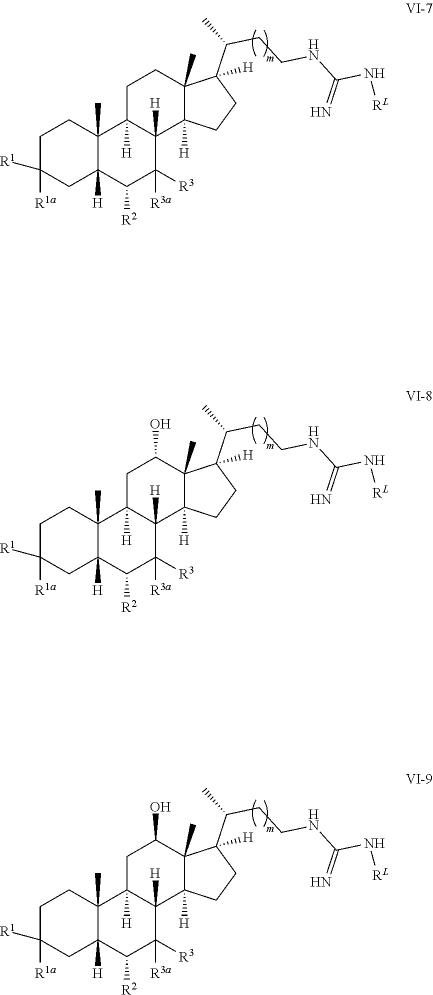

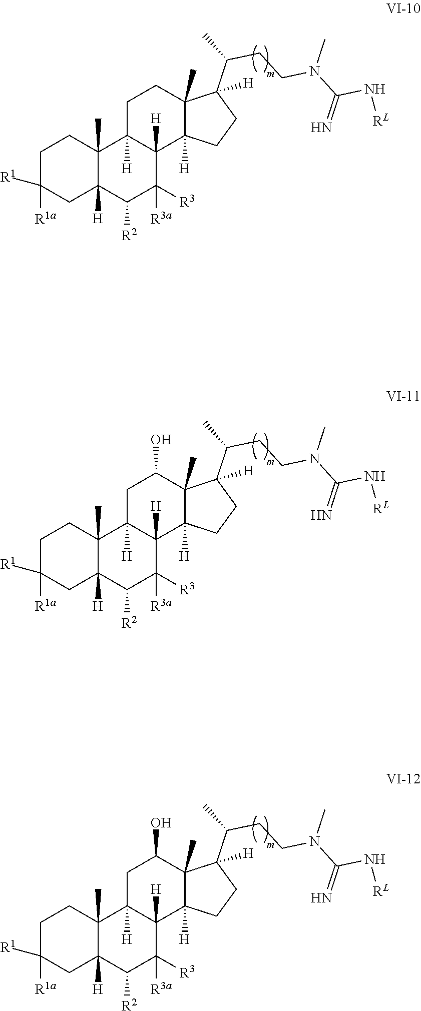

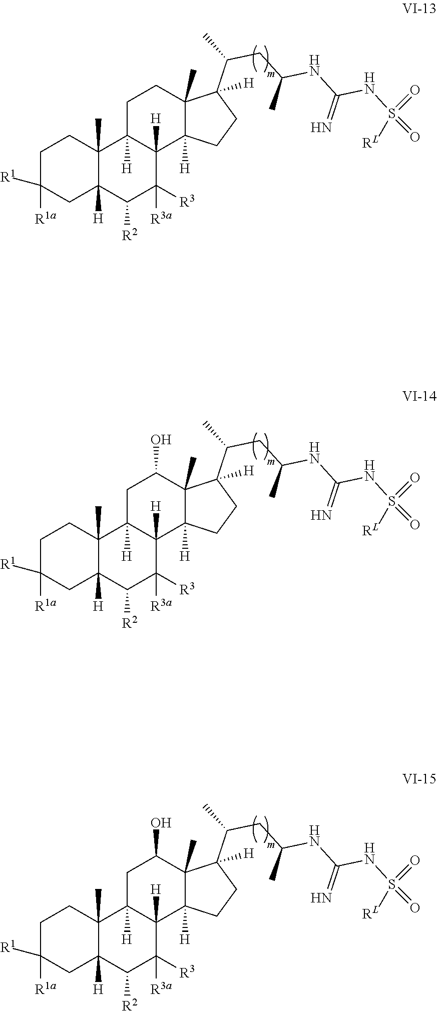

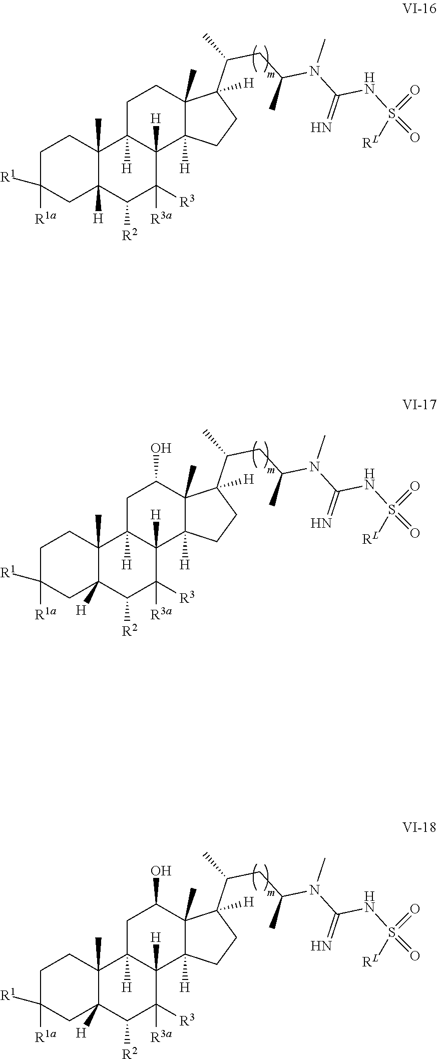

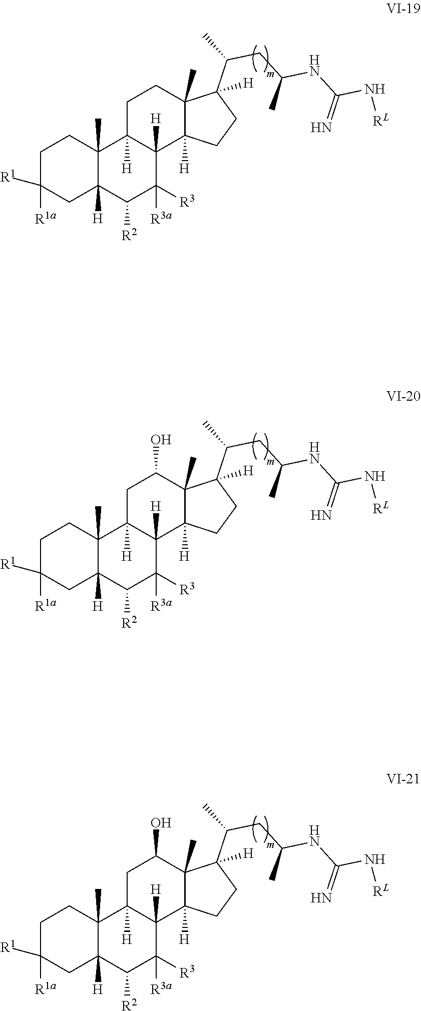

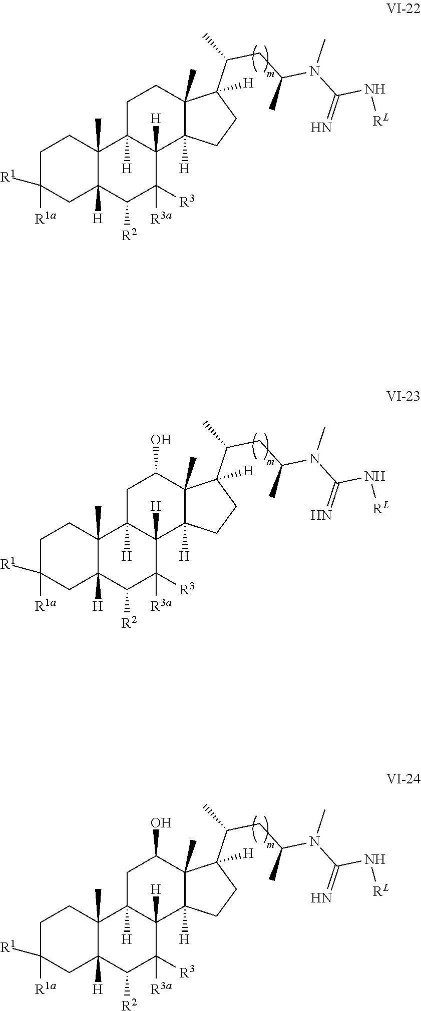

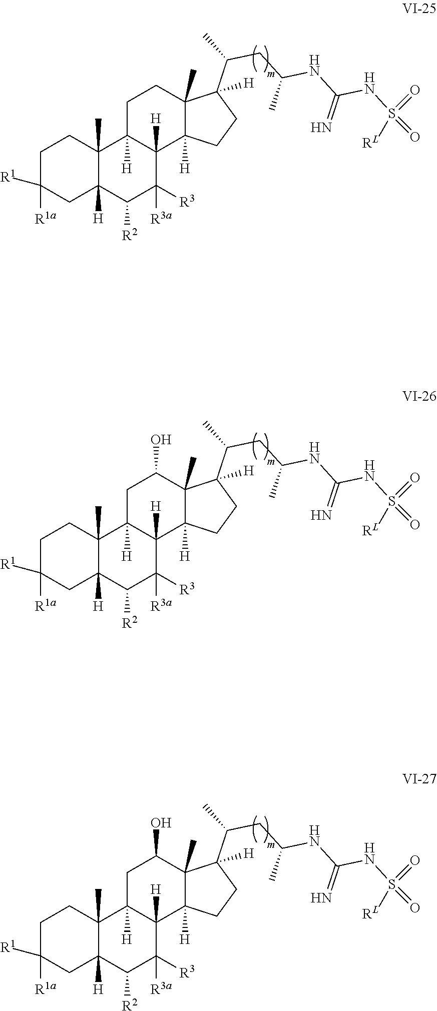

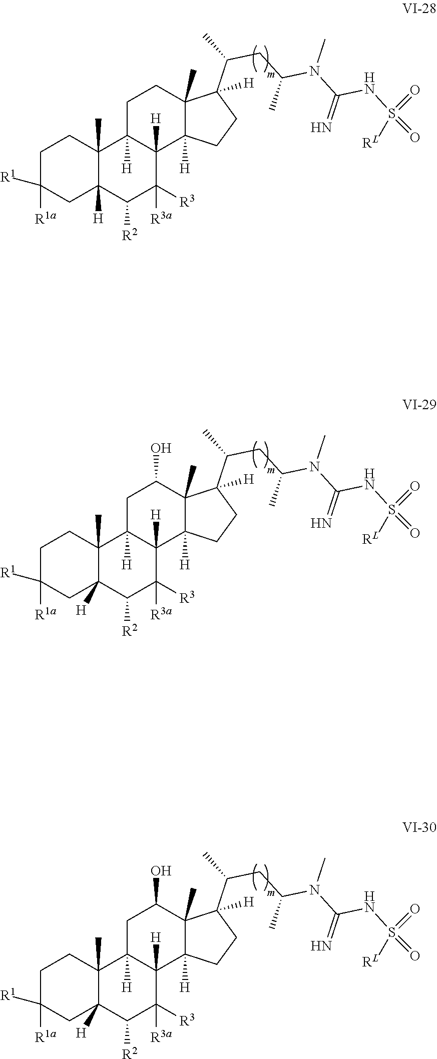

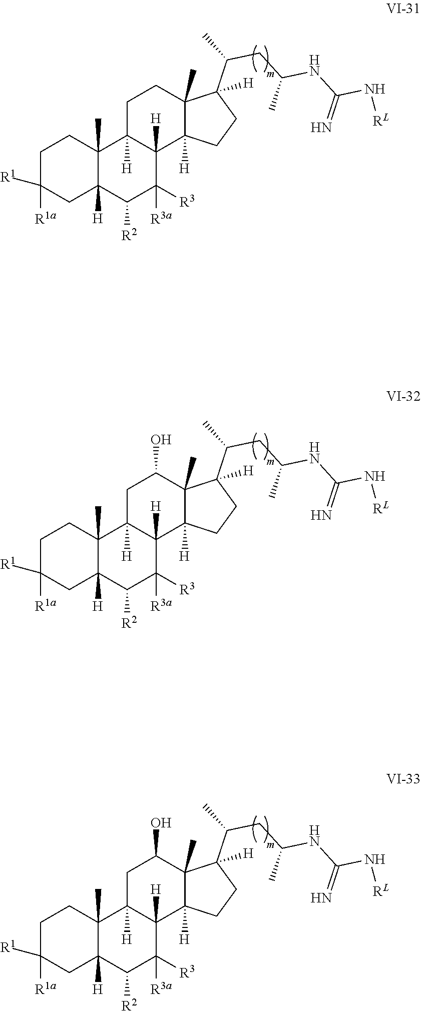

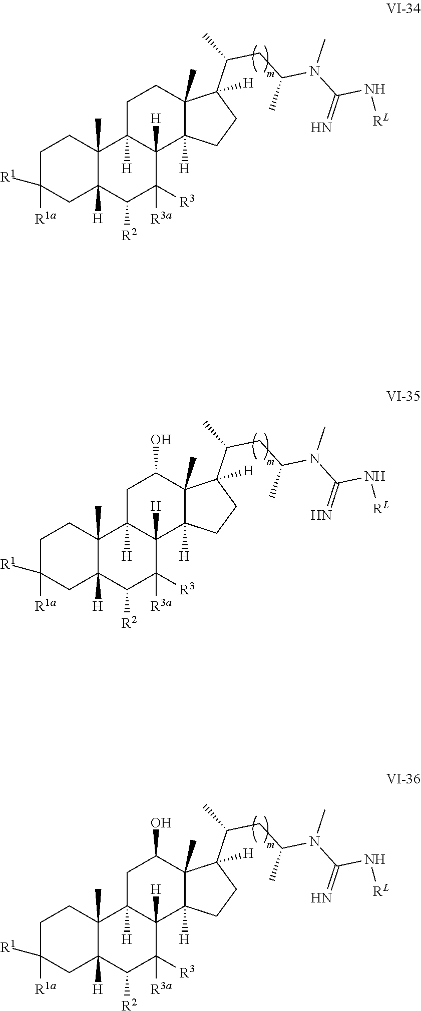

In some embodiments, R^{L} is —N(R′)C(O)N(R′)S(O)₂R^{s}. In some embodiments, R^{L} is —NHC(O)NHS(O)₂R^{s}. In some embodiments, R^{L} is —N(R′)C(O)N(R′)S(O)₂N(R^{s})₂. In some embodiments, R^{L} is —NHC(O)NHS(O)₂NHR^{s}. In some embodiments, R^{s} is R as described herein. In some embodiments, R^{L} is —N(R′)C(O)N(R′)S(O)₂R′. In some embodiments, R^{L} is —NHC(O)NHS(O)₂R′. In some embodiments, R^{L} is —N(R′)C(O)N(R′)S(O)₂N(R′)₂. In some embodiments, R^{L} is —NHC(O)NHS(O)₂NHR′. In some embodiments, R′ is R as described herein.

In some embodiments, a provided compound is a compound of formula C—I or a salt thereof:

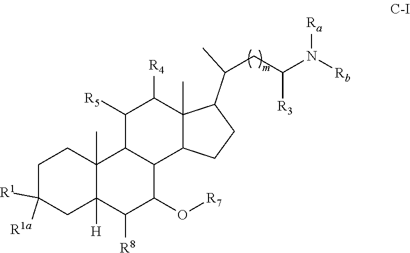

wherein:
   each of R¹ and R^{h} is independently as described herein;
   Rₐ is R^{s} as described herein;
   R₃ is R₃₀ or R^{s} as described herein;
   R_{b} is —H, —C(O)NHS(O)₂R₁, —C(O)NR₉R₁₀, or —S(O)₂R₁;
   each other variable is independently as described herein.

In some embodiments, R_{b} is —H. In some embodiments, R_{b} is —C(O)NHS(O)₂R¹. In some embodiments, R_{b} is —C(O)NR₉R₁₀. In some embodiments, R_{b} is —S(O)₂R₁.

In some embodiments, a provided compound is a compound of formula C—I′ or a salt thereof:

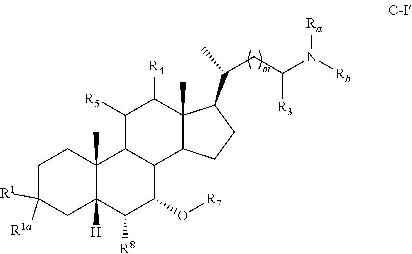

wherein each variable is independently as described herein.

In some embodiments:
each of R¹ and R^{1a} is independently as described herein;
Rₐ is —H or optionally substituted C₁₋₈ alkyl; preferably Rₐ is —H or methyl; more preferably Rₐ is —H;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
R₉ and R₁₀ are each independently —H, or an optionally substituted group selected from C₁₋₈ alkyl, C₂₋₈ alkenyl, C₂₋₈ alkynyl, C₃₋₈ cycloalkyl, and heterocyclyl (e.g., 3-15 membered heterocyclyl having 1-5 heteroatoms), or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H;
R₃ is —H or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H or —OH;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
R₈ is —H, halogen, or an optionally substituted group selected from C₁₋₈ alkyl, C₂₋₈ alkenyl, C₂₋₈ alkynyl, and C₃₋₈ cycloalkyl; preferably Rₛ is C₁₋₄ alkyl; more preferably Rₛ is ethyl.

In some embodiments:

each of R¹ and R^{1a} is independently as described herein;
Rₐ is —H;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms));
R₉ and R₁₀ are each independently —H, or an optionally substituted group selected from C₁₋₈ alkyl, C₂₋₈ alkenyl, C₂₋₈ alkynyl, and C₃₋₈ cycloalkyl, or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H;
R₃ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H or —OH;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
R₈ is —H, halogen, or an optionally substituted group selected from C₁₋₈ alkyl, C₂₋₈ alkenyl, C₂₋₈ alkynyl, and C₃₋₈ cycloalkyl; preferably Rₛ is C₁₋₄ alkyl; more preferably Rₛ is ethyl.

In some embodiments, R_{b} is —C(O)NHS(O)₂R₁. In some embodiments, R_{b} is —C(O)NR₉R₁₀. In some embodiments, In some embodiments, R_{b} is —S(O)₂R₁. In some embodiments, R_{b} is —H.

In some embodiments:
each of R¹ and R^{1a} is independently as described herein;
Rₐ is —H or optionally substituted C₁₋₈ alkyl; preferably Rₐ is —H or methyl; more preferably Rₐ is —H;
R_{b} is —C(O)NHS(O)₂R₁;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
R₉ and R₁₀ are each independently —H, or an optionally substituted group selected from C₁₋₈ alkyl, C₂₋₈ alkenyl, C₂₋₈ alkynyl, C₃₋₈ cycloalkyl, and 3-15 membered heterocyclyl having 1-5 heteroatoms, or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H;
R₃ is —H or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H or —OH;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
R₈ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably R₈ is ethyl.

In some embodiments:
each of R¹ and R^{1a} is independently as described herein;
Rₐ is —H;
R_{b} is —C(O)NHS(O)₂R¹;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms));
R₉ and R₁₀ are each independently —H, or an optionally substituted group selected from C₁₋₈ alkyl, C₂₋₈ alkenyl, C₂₋₈ alkynyl, and C₃₋₈ cycloalkyl, or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H;
R₃ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H or —OH;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
R₈ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably R₈ is ethyl.

In some embodiments, R₃, R₄, R₅, and R₇ are each —H and R₈ is ethyl.

In some embodiments, a provided compound is a compound of formula C-IA or a salt thereof:

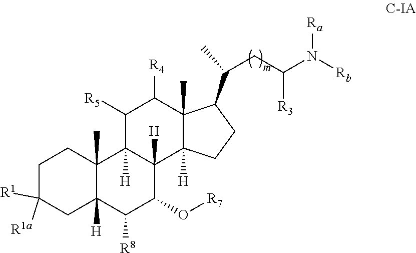

wherein each variable is independently as described herein.

In some embodiments, a provided compound is a compound of formula C-II or a salt thereof:

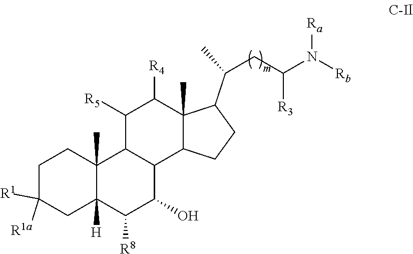

wherein each variable is independently as described herein.

In some embodiments, a provided compound is a compound of formula C—III or a salt thereof:

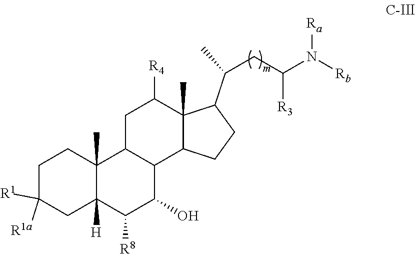

wherein each variable is independently as described herein.

In some embodiments, a provided compound is a compound of formula C—III-a or a salt thereof:

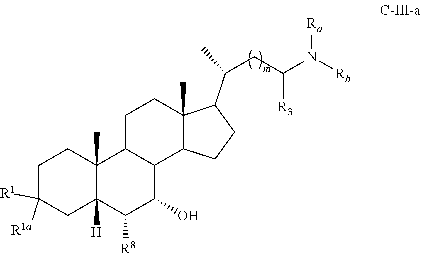

wherein each variable is independently as described herein.

In some embodiments, a provided compound is a compound of formula C—III-b or a salt thereof:

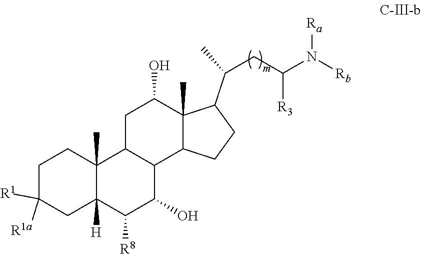

wherein each variable is independently as described herein.

In some embodiments, a provided compound is a compound of formula C-ITT-c or asalt thereof:

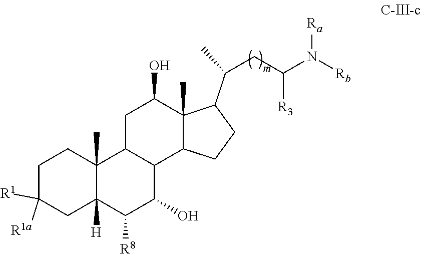

wherein each variable is independently as described herein.

In some embodiments, a compound has a structure selected from formula (III-1) to (III-54) of WO 2016/086134 or WO 2016/086218 or a salt thereof. In some embodiments, its 3-groups (e.g., 3-groups such as 3-OH and 3-H) are replaced with R¹ and R^{1M} as described herein (e.g., as in C—III-c), e.g., in some embodiments, both replaced with —H. In some embodiments, R₃ is —H. In some embodiments, R₃ is —CH₃. In some embodiments, the carbon to which R₃ attached is of S configuration. In some embodiments, the carbon to which R₃ attached is of R configuration. In some embodiments, —NRₐR_{b} is —NHC(O)NHS(O)₂R₁. In some embodiments, —NRₐR_{b} is —N(CH₃)C(O)NHS(O)₂R₁. In some embodiments, —NRₐR_{b} is —NHS(O)₂R₁. In some embodiments, —NRₐR_{b} is —N(CH₃)S(O)₂R₁. In some embodiments, —NRₐR_{b} is —NHC(O)NHR₁₀. In some embodiments, —NRₐR_{b} is —N(CH₃)C(O)NHR₁₀.

In some embodiments, a provided compound is a compound of formula C—IV-A or a salt thereof:

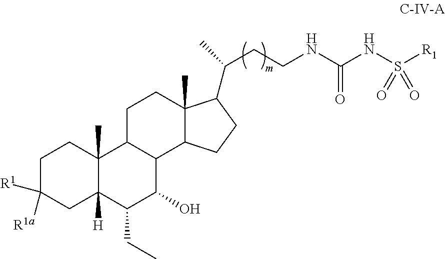

wherein each variable is independently as described herein.

In some embodiments, a provided compound is a compound of formula C-IV-B or a salt thereof:

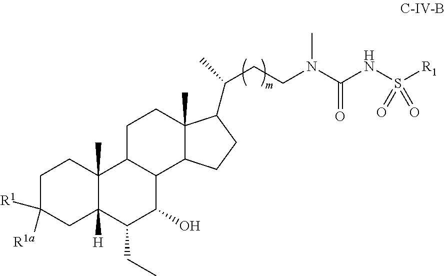

wherein each variable is independently as described herein.

In some embodiments, a provided compound is a compound of formula C-IV-C or a salt thereof:

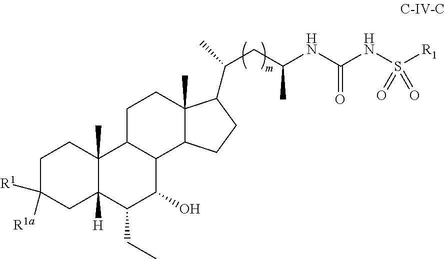

wherein each variable is independently as described herein.

In some embodiments, a provided compound is a compound of formula C—IV-D or a salt thereof:

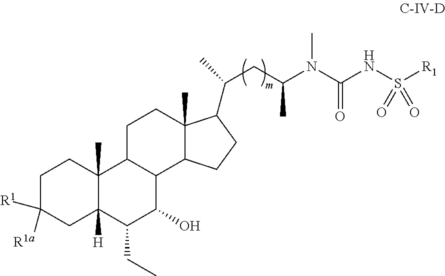

wherein each variable is independently as described herein.

In some embodiments, a provided compound is a compound of formula C—V-A or a salt thereof:

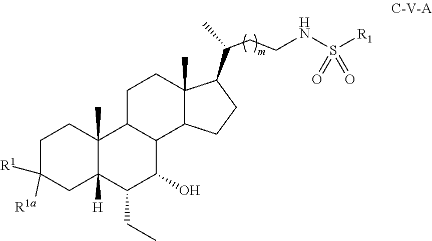

wherein each variable is independently as described herein.

In some embodiments, a provided compound is a compound of formula C-V-B or a salt thereof:

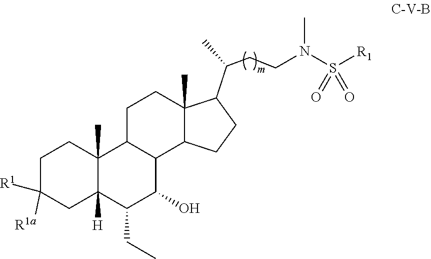

wherein each variable is independently as described herein.

In some embodiments, a provided compound is a compound of formula C—VI-A or a salt thereof:

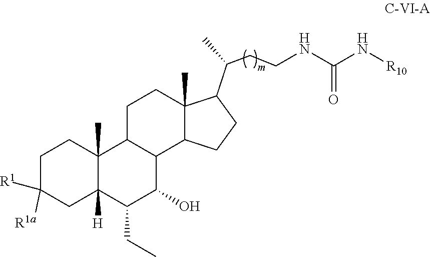

wherein each variable is independently as described herein.

In some embodiments, a provided compound is a compound of formula C-VI-B or a salt thereof:

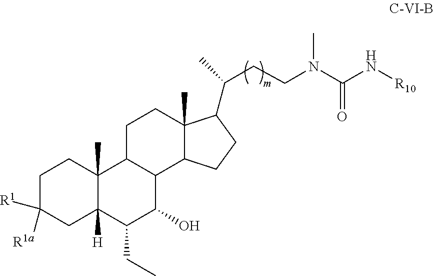

wherein each variable is independently as described herein.

In some embodiments, a provided compound is a compound of formula C—VII-A or a salt thereof:

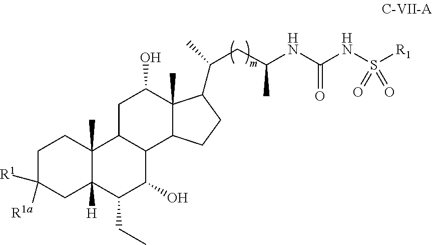

wherein each variable is independently as described herein.

In some embodiments, a provided compound is a compound of formula C-VII-B or a salt thereof:

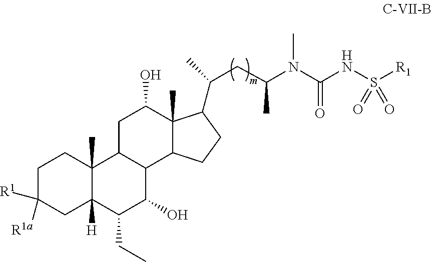

wherein each variable is independently as described herein.

In some embodiments, R^{L} is R₁. In some embodiments, R₁ is C₁₋₄ alkyl. In some embodiments, R₁ is C₁₋₄ haloalkyl. In some embodiments, R₁ is C₂₋₄ alkenyl. In some embodiments, R₁ is C₂₋₄ alkynyl. In some embodiments, R₁ is phenyl-C₁₋₄ alkyl. In some embodiments, R₁ is optionally substituted C₃₋₆ cycloalkyl. In some embodiments, R₁ is optionally substituted C₃₋₆ cycloalkyl-C₁₋₄ alkyl. In some embodiments, R₁ is 5- or 6-membered heterocyclyl. In some embodiments, R₁ is amino. In some embodiments, R₁ is optionally substituted phenyl. In some embodiments, R₁ is halogen. In some embodiments, R₁ is R as described herein. In some embodiments, R₁ is optionally substituted C₁₋₆ alkyl. In some embodiments, R₁ is methyl. In some embodiments, R₁ is ethyl. In some embodiments, R₁ is butyl. In some embodiments, R₁ is t-butyl. In some embodiments, R₁ is propyl. In some embodiments, R₁ is isopropyl. In some embodiments, R₁ is benzyl. In some embodiments, R₁ is vinyl. In some embodiments, R₁ is allyl. In some embodiments, R₁ is —CF₃. In some embodiments, R₁ is cyclopropyl. In some embodiments, R₁ is 1-methylcyclopropyl. In some embodiments, R₁ is cyclopropylmethyl. In some embodiments, R₁ is 1-pyrrolidinyl. In some embodiments, R₁ is 1-piperidinyl. In some embodiments, R₁ is 4-morpholinyl. In some embodiments, R₁ is —NH₂. In some embodiments, R₁ is dimethylamino. In some embodiments, R₁ is optionally substituted phenyl. In some embodiments, R₁ is phenyl. In some embodiments, R₁ is 4-trifluoromethoxyphenyl. In some embodiments, R₁ is 2-methylphenyl. In some embodiments, R₁ is 2-trifluoromethoxyphenyl. In some embodiments, R₁ is 2-fluorophenyl. In some embodiments, R₁ is 4-tert-butylphenyl. In some embodiments, R₁ is 2-naphthyl. In some embodiments, R₁ is 4-methylphenyl. In some embodiments, R₁ is 2-methoxyphenyl. In some embodiments, R₁ is —F.

In some embodiments, R₁ is selected from below:

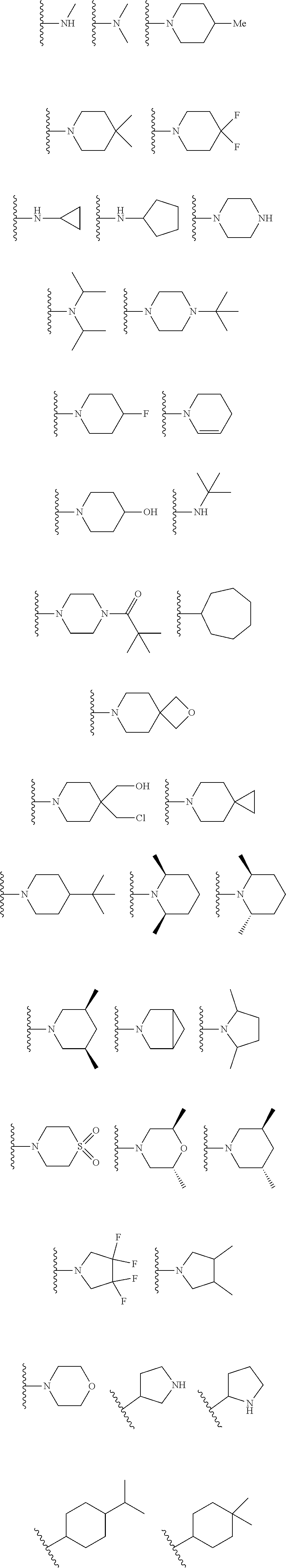

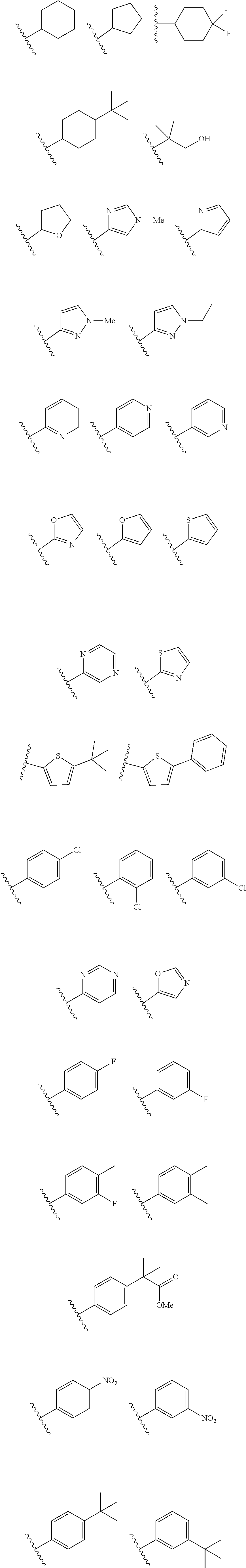

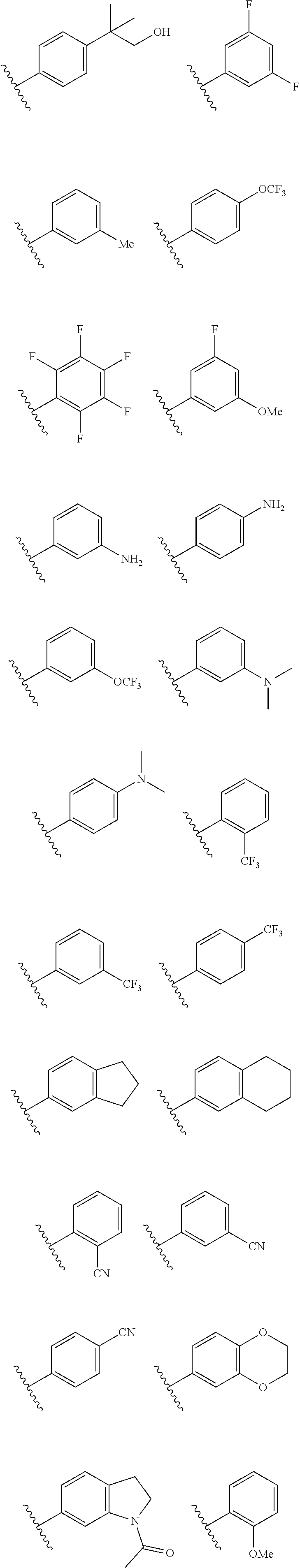

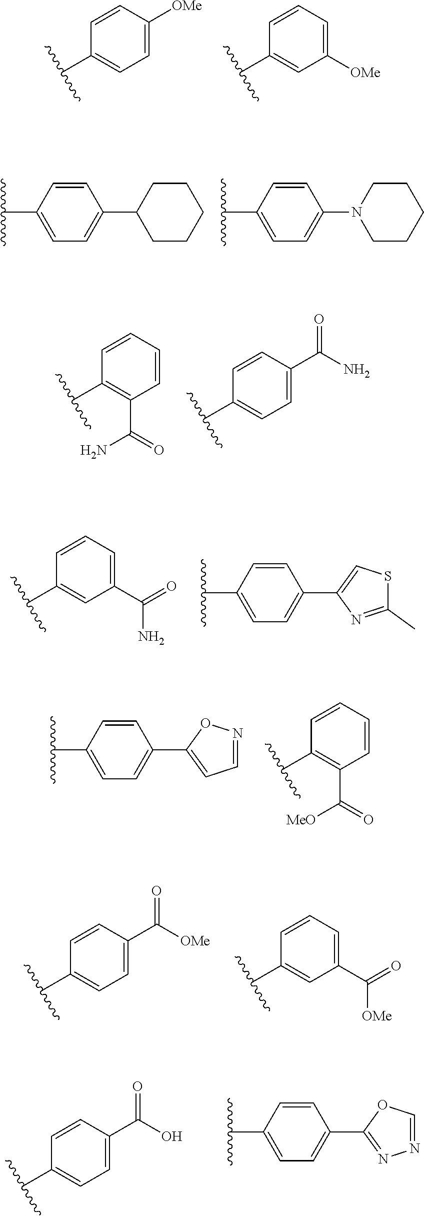

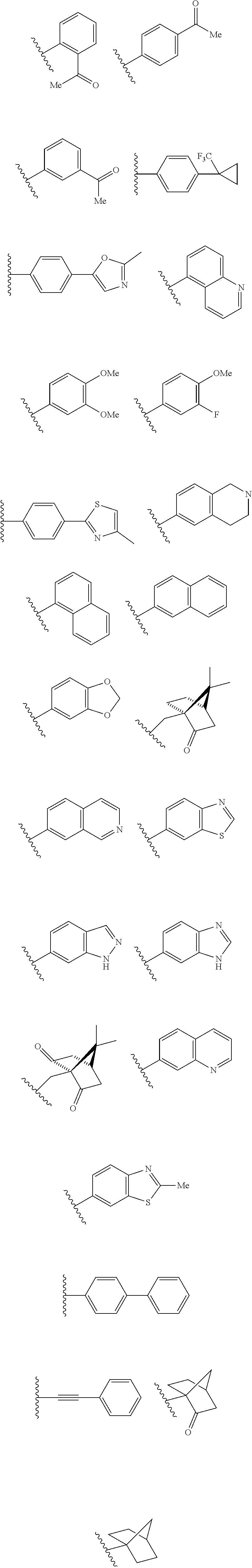

In some embodiments, R₉ is —H. In some embodiments, R₉ is —H and Rio is not —H. In some embodiments, R₁₀ is C₁₋₄ alkyl. In some embodiments, R₁₀ is C₁₋₄ haloalkyl. In some embodiments, R₁₀ is C₂₋₄ alkenyl. In some embodiments, R₁₀ is C₂₋₄ alkynyl. In some embodiments, R₁₀ is phenyl-C₁₋₄ alkyl. In some embodiments, R₁₀ is optionally substituted C₃₋₆ cycloalkyl. In some embodiments, R₁₀ is optionally substituted C₃₋₆ cycloalkyl-C₁₋₄ alkyl. In some embodiments, R₁₀ is 5- or 6-membered heterocyclyl. In some embodiments, R₁₀ is amino. In some embodiments, R₁₀ is optionally substituted phenyl. In some embodiments, R₁₀ is R as described herein. In some embodiments, R₁₀ is optionally substituted C₁₋₆ alkyl. In some embodiments, R₁₀ is methyl. In some embodiments, R₁₀ is ethyl. In some embodiments, R₁₀ is butyl. In some embodiments, R₁₀ is t-butyl. In some embodiments, R₁₀ is propyl. In some embodiments, R₁₀ is isopropyl. In some embodiments, R₁₀ is benzyl. In some embodiments, R₁₀ is vinyl. In some embodiments, R₁₀ is allyl. In some embodiments, R₁₀ is —CF₃. In some embodiments, R₁₀ is cyclopropyl. In some embodiments, R₁₀ is 1-methylcyclopropyl. In some embodiments, R₁₀ is cyclopropylmethyl. In some embodiments, R₁₀ is 1-pyrrolidinyl. In some embodiments, R₁₀ is 1-piperidinyl. In some embodiments, R₁₀ is 4-morpholinyl. In some embodiments, R₁₀ is —NH₂. In some embodiments, R₁₀ is dimethylamino. In some embodiments, R₁₀ is optionally substituted phenyl. In some embodiments, R₁₀ is phenyl. In some embodiments, R₁₀ is 4-trifluoromethoxyphenyl. In some embodiments, R₁₀ is 2-methylphenyl. In some embodiments, R₁₀ is 2-trifluoromethoxyphenyl. In some embodiments, R₁₀ is 2-fluorophenyl. In some embodiments, R₁₀ is 4-tert-butylphenyl. In some embodiments, R₁₀ is 2-naphthyl. In some embodiments, R₁₀ is 4-methylphenyl. In some embodiments, R₁₀ is 2-methoxyphenyl. In some embodiments, R₁₀ is —H.

In some embodiments, m is 0, and R₁ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, 2-methoxyphenyl, or —F. In some embodiments, m is 1, and R₁ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, 2-methoxyphenyl or —F. In some embodiments, m is 2, and R₁ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, 2-methoxyphenyl or —F.

In some embodiments, m is 0, and R₁₀ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, —H, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, or 2-methoxyphenyl. In some embodiments, m is 1, and R₁₀ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, —H, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, or 2-methoxyphenyl. In some embodiments, m is 2, and R₁₀ is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, —H, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, or 2-methoxyphenyl.

In some embodiments, m and R₁, or m and R₁₀, are described as in Tables 1-10 of WO 2016/086134 or WO 2016/086218.

Various additional embodiments of Rₐ, R_{b}, R₃, R₄, R₅, R₇, and R₈ are described in WO 2016/086134 or WO 2016/086218 (e.g., described as Rₐ, R_{b}, R₂, R₃, R₄, R₆, and R₇, respectively, in WO 2016/086134 or WO 2016/086218), either individually or in combination (e.g., as in compounds).

In some embodiments, a provided compound is a compound described in WO 2016/086134, wherein its 3-substitutents attached to moiety A are replaced with R¹ and R^{1a}, e.g., in some embodiments, both replaced with —H. In some embodiments, a provided compound is a compound described in WO 2016/086218, wherein its 3-substitutents attached to moiety A are replaced with R¹ and R^{1a}, e.g., in some embodiments, both replaced with —H.

In some embodiments, a provided compound is a compound selected from compounds in compounds 1-750, Tables 1-11, Schemes 1-4, and Examples 1-121 of WO 2016/086134 or WO 2016/086218, wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2016/086169, e.g., a compound of formula (IA) or (IB) or a salt (including stereoisomer, solvate, hydrate or combination thereof) as described in WO 2016/086169, wherein —OR₅ in WO 2016/086169 (in various embodiments, —OR₅ is —OH in WO 2016/086169) is replaced with R^{1a} as described herein. In some embodiments, —OR₅ in WO 2016/086169 and the —H that is attached to the same carbon as such —OR₅ are replaced with R¹ and R^{1a} as described herein, e.g., in some embodiments, both replaced with —H. In some embodiments, one of R² and R^{2a} is —H and the other is R₈ as described herein. In some embodiments, one of R³ and R^{3a} is —H and the other is —OR₇ as described herein. In some embodiments, one of R⁴ and R^{4a} is —H and the other is R₄ as described herein. In some embodiments, one of R¹⁴ and R^{14a} is —H and the other is R₅ as described herein. In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein. In some embodiments, R¹³ is R as described herein. In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, in a provided compound L¹ is -L^{s0}-L¹-, wherein each variable is independently as described herein. In some embodiments, L¹ is -L^{s0}-L^{s1}-L^{s2}-, wherein each variable is independently as described herein. In some embodiments, L^{s0} is optionally substituted —CH₂—. In some embodiments, L^{s0} is —C(R)₂—. In some embodiments, L^{s0} is —CHR′—. In some embodiments, L^{s0} is —CH₂—. In some embodiments, L^{s0} is —CH(CH₃)—. In some embodiments, L^{s0} is optionally substituted —CH₂—, and L^{s1} is optionally substituted C₁-C₆ alkylene. In some embodiments, L¹ is -L^{s0}-L^{s1}-L^{s2}-, wherein L^{s0} is optionally substituted —CH₂—, L^{s1} is C₁-C₆ alkylene. In some embodiments, L¹ is -L^{s0}-L^{s1a}-L^{s1b}-L^{s2}. In some embodiments, L^{s1} is -L^{s1a}-L^{s1b}-, wherein L^{s1a} is a covalent bond, or an optionally substituted, bivalent C₁-C₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—; and L^{s1b} is a covalent bond, or an optionally substituted methylene which is optionally replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —S(O)—, —S(O)₂—, —S(O)₂N(R′)—, —C(O)S—, or —C(O)O—. In some embodiments, Lsia is optionally substituted C₁-C₅ alkylene. In some embodiments, L^{s1a} is C₁-C₅ alkylene. In some embodiments, L^{s1b} is optionally substituted —CH₂—. In some embodiments, L^{s1b} is —C(R)₂—. In some embodiments, L^{s1b} is —CHR′—. In some embodiments, L^{s1b} is —CH₂—. In some embodiments, L^{s1b} is —CH(CH₃)—.

In some embodiments, L¹ is —CHR′—(CH₂)ₘ—CH₂—CHR′-L^{s2}-. In some embodiments, L¹ is —CHR′—(CH₂)ₘ—CH₂-L^{s2}-. In some embodiments, L¹ is -L^{s0}-(CH₂)ₘ—CHR′-L^{s2}-, wherein L^{s0} is optionally substituted —CH₂—. In some embodiments, L¹ is -L^{s0}-(CH₂)ₘ—CH₂-L^{s2}-, wherein L^{s0} is optionally substituted —CH₂—. In some embodiments, L^{s0} is —CH(CH₃)—. In some embodiments, m is 0. In some embodiments, m is 1. In some embodiments, m is 2.

In some embodiments, L^{s2} is —C(O)N(R′)—. In some embodiments, L^{s2} is —C(O)N(OR)—. In some embodiments, L^{s2} is —C(O)N(R′)—O—. In some embodiments, L^{s2} is —S(O)₂—. In some embodiments, L^{s2} is —S(O)₂—O—. In some embodiments, L^{s2} is —C(O)N(R′)—. In some embodiments, L^{s2} is —C(O)NH—. In some embodiments, L^{s2} is —S(O)₂—. In some embodiments, L^{s2} is —S(O)₂—N(R′)—. In some embodiments, L^{s2} is —P(O)(OR′)—. In some embodiments, L^{s2} is —P(O)(OR′)O—. In some embodiments, L^{s2} is or comprises -Cy-. In some embodiments, L^{s2} is -Cy-. In some embodiments, L^{s2} is -Cy-O—. In some embodiments, L^{s2} is —C(O)-Cy-. In some embodiments, L^{s2} is —C(O)N(R′)C(R′)₂C(O)N(R′)—. In some embodiments, L^{s2} is —C(O)N(R′)C(R′)₂C(O)N(OR)—. In some embodiments, L^{s2} is —C(O)N(R′)C(R′)₂C(O)N(R′)—O—. In some embodiments, L^{s2} is —C(O)N(R′)C(R′)₂S(O)₂—. In some embodiments, L^{s2} is —C(O)N(R′)C(R′)₂S(O)₂—O—. In some embodiments, L^{s2} is —C(O)N(R′)C(R′)₂C(O)N(R′)—. In some embodiments, L^{s2} is —C(O)N(R′)C(R′)₂C(O)NH—. In some embodiments, L^{s2} is —C(O)N(R′)C(R′)₂S(O)₂—. In some embodiments, L^{s2} is —C(O)N(R′)C(R′)₂S(O)₂—N(R′)—. In some embodiments, L^{s2} is —C(O)N(R′)C(R′)₂P(O)(OR′)—. In some embodiments, L^{s2} is —C(O)N(R′)C(R′)₂P(O)(OR′)O—. In some embodiments, L^{s2} is —C(O)N(R′)C(R′)₂Cy-. In some embodiments, L^{s2} is —C(O)N(R′)C(R′)₂Cy-O—. In some embodiments, L^{s2} is —C(O)N(R′)C(R′)₂C(O)-Cy-. In some embodiments, -Cy- is an optionally substituted heteroaryl ring as described herein. In some embodiments, -Cy- is an optionally substituted 5-membered heteroaryl ring having 1-4 heteroatoms. In some embodiments, a heteroatom is nitrogen. In some embodiments, a heteroatom is oxygen. In some embodiments, there are two heteroatoms and one is oxygen and the other is oxygen. In some embodiments, there are three heteroatoms, two of which are nitrogen and the other is oxygen. In some embodiments, there are three heteroatoms, two of which are nitrogen and the other is sulfur. In some embodiments, there are four heteroatoms each of which is nitrogen. In some embodiments, there are two heteroatoms and each is nitrogen. In some embodiments, there are two heteroatoms, one of which is nitrogen and the other is sulfur. In some embodiments, there is one heteroatom. In some embodiments, a heteroatom is nitrogen. In some embodiments, a heteroatom is oxygen. In some embodiments, a heteroatom is sulfur. In some embodiments, -Cy- is an optionally substituted C₃₋₆ cycloaliphatic ring. In some embodiments, -Cy- is an optionally substituted cyclopentyl ring. In some embodiments, -Cy- is an optionally substituted cyclobutenyl ring. In some embodiments, -Cy- is optionally substituted phenyl ring. In some embodiments, -Cy- is a phenyl ring.

In some embodiments, L¹ is —CH(CH₃)(CH₂)mCH₂—. In some embodiments, L¹ is —CH(CH₃)(CH₂)mCH₂—C(O)NHCH₂—. In some embodiments, L¹ is —CH(CH₃)(CH₂)mCH(CH₃)—. In some embodiments, L¹ is —CH(CH₃)(CH₂)mCH(CH₃)—C(O)NHCH₂—.

In some embodiments, L^{s0} is —CH(R′)— or substituted —CH₂—, wherein the carbon atom is chiral and is S. In some embodiments, L^{s0} is —CH(R′)— or substituted —CH₂—, wherein the carbon atom is chiral and is R. In some embodiments, L^{s1b} is —CH(R′)— or substituted —CH₂—, wherein the carbon atom is chiral and is S. In some embodiments, L^{s1b} is —CH(R′)— or substituted —CH₂—, wherein the carbon atom is chiral and is R. In some embodiments, each of L^{s0} and L^{s1b} is independently —CH(R′)— or substituted —CH₂— wherein the carbon atom is chiral, and the configurations of the two chiral carbon atoms (the first for L^{s0} and the second for L^{s1b}) is SS. In some embodiments, they are SR. In some embodiments, they are R^{s}. In some embodiments, they are RR.

In some embodiments, R^{L} is R. In some embodiments, R is —H. In some embodiments, R^{L} is —OR′. In some embodiments, R^{L} is —OH. In some embodiments, R^{L} is —N(R^{s})₂. In some embodiments, R^{L} is —N(R′)₂. In some embodiments, R^{L} is —N(OR)R^{s}. In some embodiments, R^{L} is —N(OR)R′. In some embodiments, R^{L} is —N(OR)R. In some embodiments, R^{L} is —C(O)N(R^{s})₂. In some embodiments, R^{L} is —C(O)N(R′)₂. In some embodiments, R^{L} is —C(O)N(OR′)R^{s}. In some embodiments, R^{L} is —C(O)N(OR)R^{s}. In some embodiments, R^{L} is —C(O)N(OR)R′. In some embodiments, R^{L} is —C(O)N(R^{s})OH. In some embodiments, R^{L} is —C(O)N(R′)OH. In some embodiments, R^{L} is —C(O)N(OR)R. In some embodiments, R^{L} is —S(O)₂R′. In some embodiments, R^{L} is —SO₃R. In some embodiments, R^{L} is —SO₃H. In some embodiments, R^{L} is —C(O)N(R^{s})CN. In some embodiments, R^{L} is —C(O)N(R′)CN. In some embodiments, R^{L} is —C(O)NH—CN. In some embodiments, R^{L} is —S(O)₂N(R′)₂. In some embodiments, R^{L} is R. In some embodiments, R^{L} is —C(O)R. In some embodiments, R is optionally substituted heteroaryl as described herein. In some embodiments, R is optionally substituted 5-membered heteroaryl having 1-4 heteroatoms. In some embodiments, a heteroatom is nitrogen. In some embodiments, a heteroatom is oxygen. In some embodiments, there are two heteroatoms and one is oxygen and the other is oxygen. In some embodiments, there are three heteroatoms, two of which are nitrogen and the other is oxygen. In some embodiments, there are three heteroatoms, two of which are nitrogen and the other is sulfur. In some embodiments, there are four heteroatoms each of which is nitrogen. In some embodiments, there are two heteroatoms and each is nitrogen. In some embodiments, there are two heteroatoms, one of which is nitrogen and the other is sulfur. In some embodiments, there is one heteroatom. In some embodiments, a heteroatom is nitrogen. In some embodiments, a heteroatom is oxygen. In some embodiments, a heteroatom is sulfur. In some embodiments, R is optionally substituted C₃₋₆ cycloaliphatic. In some embodiments, R is optionally substituted C₃₋₆ cycloalkyl. In some embodiments, R is optionally substituted cyclopentyl. In some embodiments, R is optionally substituted cyclobutenyl. In some embodiments, R is optionally substituted phenyl. In some embodiments, R is phenyl.

In some embodiments, R^{L} is —C(O)N(R′)C(R′)₂C(O)N(R^{s})₂. In some embodiments, R^{L} is —C(O)N(R′)C(R′)₂C(O)N(R′)₂. In some embodiments, R^{L} is —C(O)N(R′)C(R′)₂S(O)₂R^{s}. In some embodiments, R^{L} is —C(O)N(R′)C(R′)₂S(O)₂R′. In some embodiments, R^{L} is —C(O)N(R′)C(R′)₂S(O)₂N(R^{s})₂. In some embodiments, R^{L} is —C(O)N(R′)C(R′)₂S(O)₂N(R′)₂. In some embodiments, R^{L} is —C(O)N(R′)C(R′)₂P(O)(R^{s})₂. In some embodiments, R^{L} is —C(O)N(R′)C(R′)₂P(O)(R′)₂. In some embodiments, R^{L} is —C(O)N(R′)C(R′)₂N(R′)C(O)N(R′)S(O)₂R^{s}. In some embodiments, R^{L} is —C(O)N(R′)C(R′)₂N(R′)C(O)N(R′)S(O)₂R′. In some embodiments, R^{L} is —C(O)N(R′)C(R^{s})₃. In some embodiments, R^{L} is —C(O)N(R′)C(R′)₃. In some embodiments, R^{L} is —C(O)N(R′)C(R′)₂C(O)N(R′)S(O)₂R^{s}. In some embodiments, R^{L} is —C(O)N(R′)C(R′)₂C(O)N(R′)S(O)₂R′. In some embodiments, R^{s} is R′. In some embodiments, R^{s} is R.

In some embodiments, R^{L} is selected from:

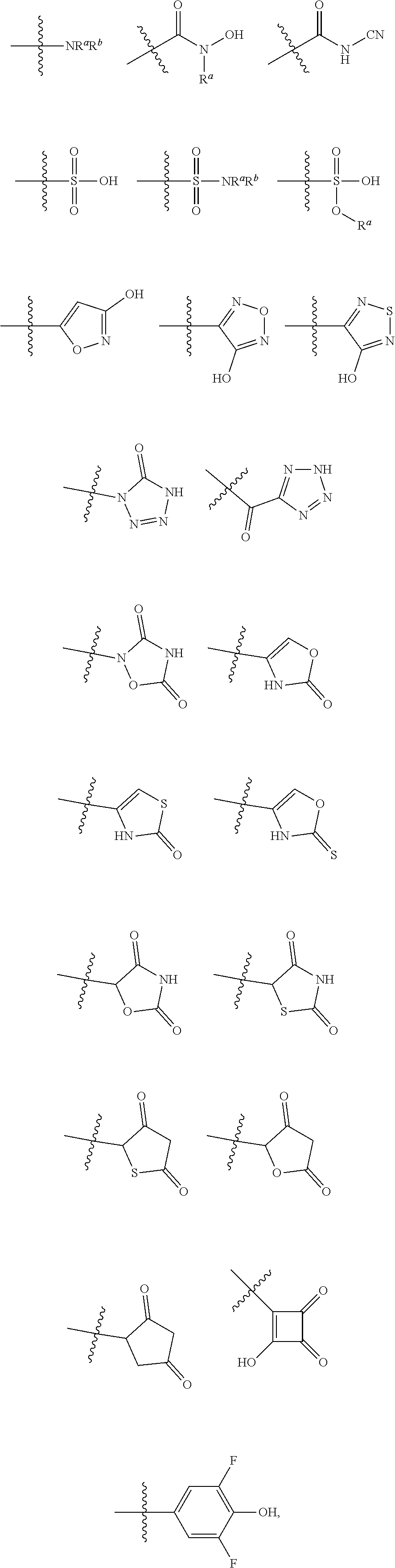

wherein each of R^{a} and R^{b} is independently R^{s} as described herein. In some embodiments, R^{L} is selected form:

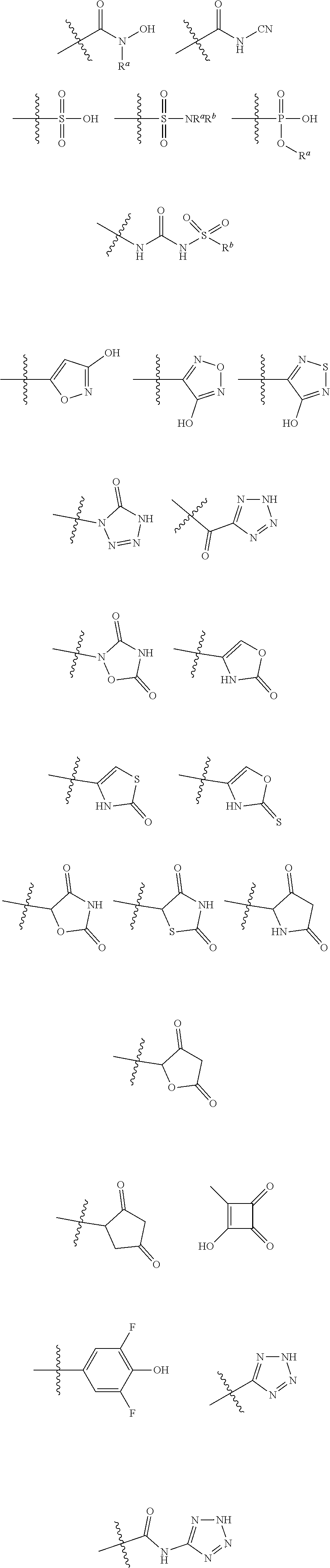

wherein each of R^{a} and R^{b} is independently R^{s} as described herein. In some embodiments, R^{a} is R as described herein. In some embodiments, R is —H or optionally substituted C₁₋₁₀ aliphatic. In some embodiments, R^{a} is —H or optionally substituted C₃₋₈ alkyl. In some embodiments, R^{a} is optionally substituted C₃₋₈ alkyl. In some embodiments, R^{a} is optionally substituted C₃₋₈ cycloalkyl. In some embodiments, R is R₁ as described herein. In some embodiments, R is halogen, —OH, —N(R′)₂, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms)). In some embodiments, R is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms)); wherein R₉ and R₁₀ are each independently —H, or an optionally substituted group selected from C₁₋₈ alkyl, C₂₋₈ alkenyl, C₂₋₈ alkynyl, and C₃₋₈ cycloalkyl, or R₉ and R₁₀ are taken together with the nitrogen to which they are attached to form an optionally substituted 3-15 membered, monocyclic, bicyclic or polycyclic heterocyclyl ring having 1-5 heteroatoms. In some embodiments, R^{L} is R¹⁵ as described herein.

In some embodiments, a provided compound is a compound of formula D-I or a salt thereof:

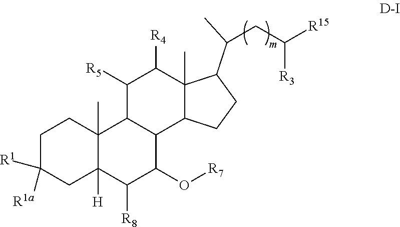

wherein:
   R¹⁵ is R^{L};

each other variable is independently as described herein.

In some embodiments, a provided compound is a compound of formula C-IA or a salt thereof:

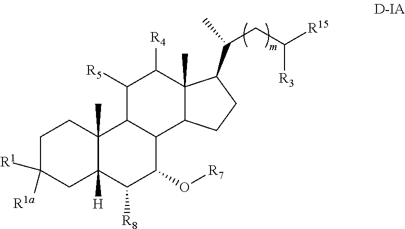

wherein each variable is independently as described herein.

In some embodiments, R¹⁵ is selected from:

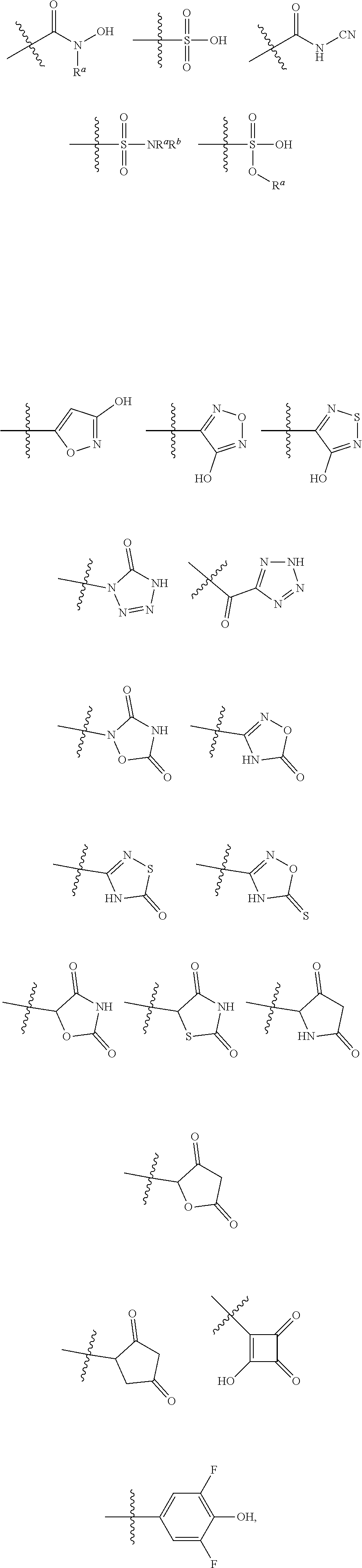

wherein:
   Rₐ is —H or an optionally substituted group selected from C₁₋₈ alkyl and C₃₋₈ cycloalkyl;
   R^{b} is —H, halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms));
   R₉ and R₁₀ are each independently —H, or an optionally substituted group selected from C₁₋₈ alkyl, C₂₋₈ alkenyl, C₂₋₈ alkynyl, C₃₋₈ cycloalkyl, and 3-15 membered heterocyclyl having 1-5 heteroatoms, or R₉ and R₁₀ are taken together with the nitrogen to which they are attached to form an optionally substituted 3-15 membered, monocyclic, bicyclic or polycyclic heterocyclyl ring having 1-5 heteroatoms;
   R₃ is —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl); preferably R₃ is —H or methyl;
   m is 0, 1, 2, or 3; preferably m is 0, 1, or 2;
   R₄ is —H, —OH, —OSO₃H, —OAc or —OPO₃H₂; preferably R₄ is —H or —OH;
   R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
   R₇ is —H or a hydroxyl protecting group (e.g., acetyl, trimethylsilyl, or benzyl); preferably R₇ is —H; and
   R₈ is —H, halogen, or an optionally substituted group selected from C₁₋₈ alkyl, C₂₋₈ alkenyl, C₂₋₈ alkynyl, and C₃₋₈ cycloalkyl; preferably Rₛ is C₁₋₄ alkyl; more preferably Rₛ is ethyl.

In some embodiments, R^{L} is —C(O)N(R₂)CH(R¹⁶)(R^{16a}), wherein each of R₂, R¹⁶ and R^{16a} is independently as described herein. In some embodiments, R₂ is R^{s}. In some embodiments, R₂ is R′. In some embodiments, R₂ is R. In some embodiments, R₂ is H. In some embodiments, R^{L} is R¹⁶ as described herein.

In some embodiments, a provided compound is a compound of formula D-I′ or a salt thereof:

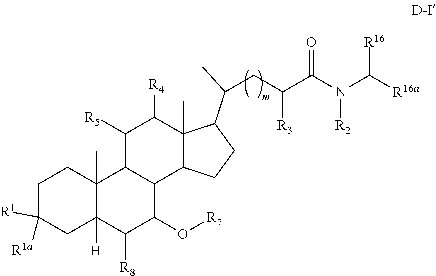

wherein:
   R¹⁶ is R^{L};
   R^{16a} is R^{s}; and
   each other variable is independently as described herein.

In some embodiments, a provided compound is a compound of formula C-IA or a salt thereof:

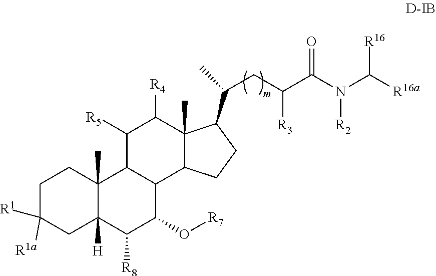

wherein each variable is independently as described herein.

In some embodiments, R¹⁶ is selected from:

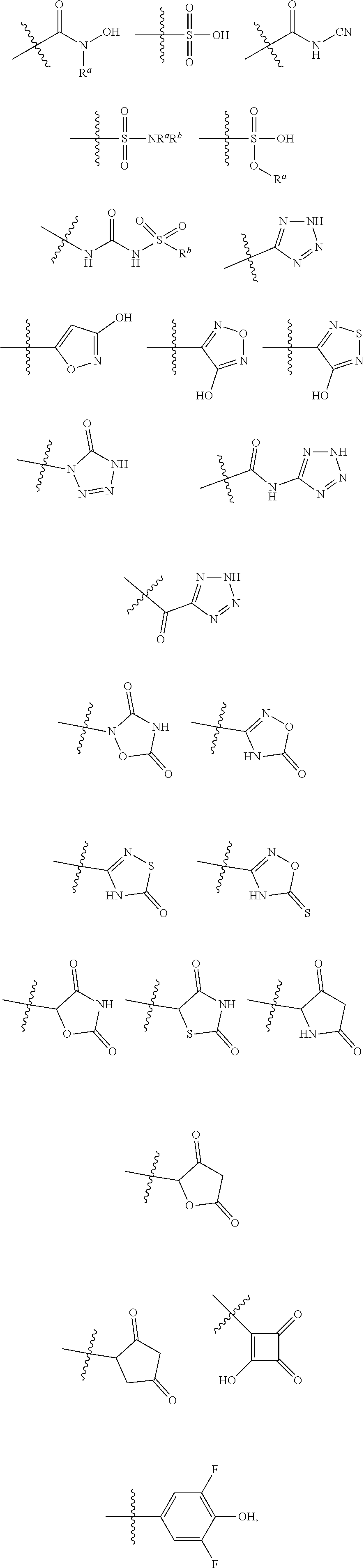

wherein:
   Rₐ is —H or an optionally substituted group selected from C₁₋₈alkyl and C₃₋₈ cycloalkyl;
   R^{b} is —H, halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), heterocyclyl (e.g., 3-15 membered heterocyclyl having 1-5 heteroatoms), heteroaryl (e.g., 5-14 membered heteroaryl having 1-10 heteroatoms), and alkylheteroaryl (C₁₋₁₂alkyl(5-14 membered heteroaryl having 1-10 heteroatoms));
   R₉ and R₁₀ are each independently —H, or an optionally substituted group selected from C₁₋₈ alkyl, C₂₋₈ alkenyl, C₂₋₈ alkynyl, C₃₋₈ cycloalkyl, and 3-15 membered heterocyclyl having 1-5 heteroatoms, or R₉ and R₁₀ are taken together with the nitrogen to which they are attached to form an optionally substituted 3-15 membered, monocyclic, bicyclic or polycyclic heterocyclyl ring having 1-5 heteroatoms;
   each of R₂, R₃ and R^{16a} is independently —H or an optionally substituted group selected from C₁₋₈ alkyl, C₂₋₈ alkenyl, C₂₋₈ alkynyl, aryl (e.g., C₆₋₁₄ aryl), and alkylaryl (e.g., C₁₋₁₂alkylC₆₋₁₄aryl); preferably R₃ is —H or methyl;
   m is 0, 1, 2, or 3; preferably m is 0, 1, or 2;
   R₄ is —H, —OH, —OSO₃H, —OAc or —OPO₃H₂; preferably R₄ is —H or —OH;
   R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
   R₇ is —H or a hydroxyl protecting group (e.g., acetyl, trimethylsilyl, or benzyl); preferably R₇ is —H; and
   R₈ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably R₈ is ethyl.

In some embodiments, R₂ is —H. In some embodiments, R^{16a} is —H. In some embodiments, both R₂ and R^{16a} are —H. In some embodiments, R₃ is —H or —CH₃, m is 0, 1, or 2, R₄ is —H or —OH, R₈ is —H, R₇ is —H, Rₛ is ethyl, R₂ is —H and R^{16a} is —H.

In some embodiments, R^{a} is —H, C₄₋₆ cycloalkyl, phenyl-C₁₋₄ alkyl or C₁₋₄ alkyl. In some embodiments, R^{a} is benzyl, C₁₋₃ alkyl or C₄₋₆ cycloalkyl.

In some embodiments, a provided compound is a compound of formula D-IA′ or a salt thereof:

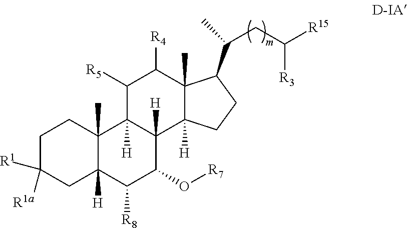

wherein each variable is independently as described herein.

In some embodiments, a provided compound is a compound of formula D-IB′ or a salt thereof:

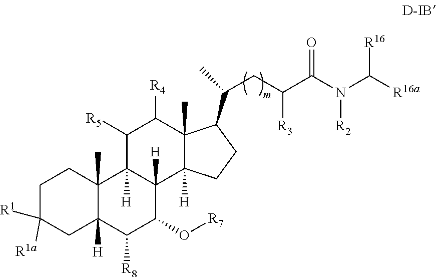

wherein each variable is independently as described herein.

In some embodiments, a provided compound is a compound of formula D-III-A or a salt thereof:

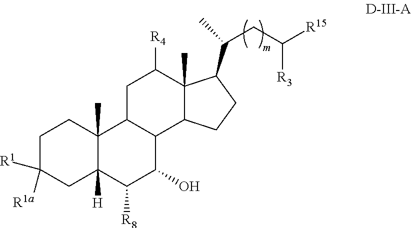

wherein each variable is independently as described herein.

In some embodiments, a provided compound is a compound of formula D-III-B or a salt thereof:

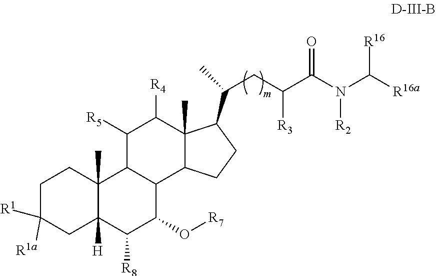

wherein each variable is independently as described herein.

In some embodiments, a provided compound is a compound of formula D-IV-A or a salt thereof:

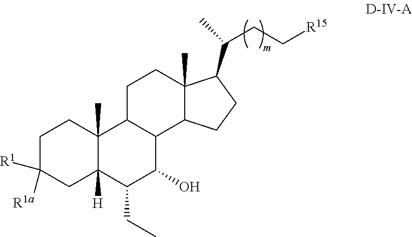

wherein each variable is independently as described herein.

In some embodiments, a provided compound is a compound of formula D-IV-B or a salt thereof:

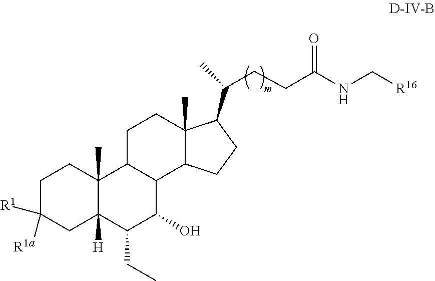

wherein each variable is independently as described herein.

In some embodiments, a provided compound is a compound of formula D-V-A or a salt thereof:

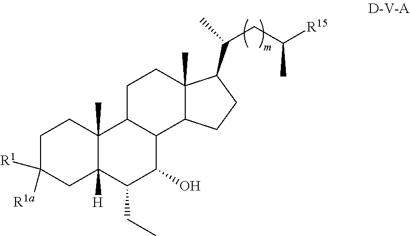

wherein each variable is independently as described herein.

In some embodiments, a provided compound is a compound of formula D-V-B or a salt thereof:

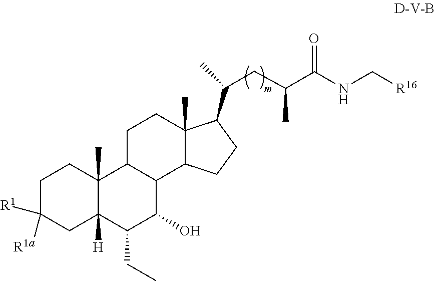

wherein each variable is independently as described herein.

In some embodiments, a provided compound is a compound of formula D-VI-A or a salt thereof:

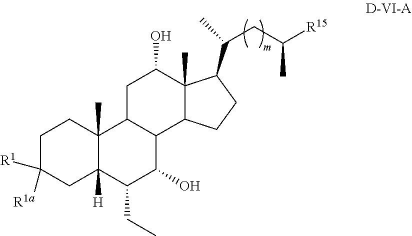

wherein each variable is independently as described herein.

In some embodiments, a provided compound is a compound of formula D-VI-B or a salt thereof:

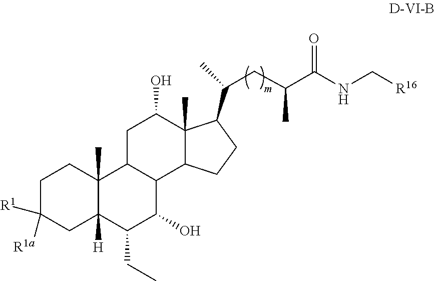

wherein each variable is independently as described herein.

In some embodiments, a provided compound is a compound of formula XI or a salt thereof:

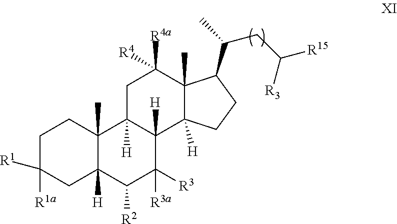

wherein:
   R¹⁵ is selected from

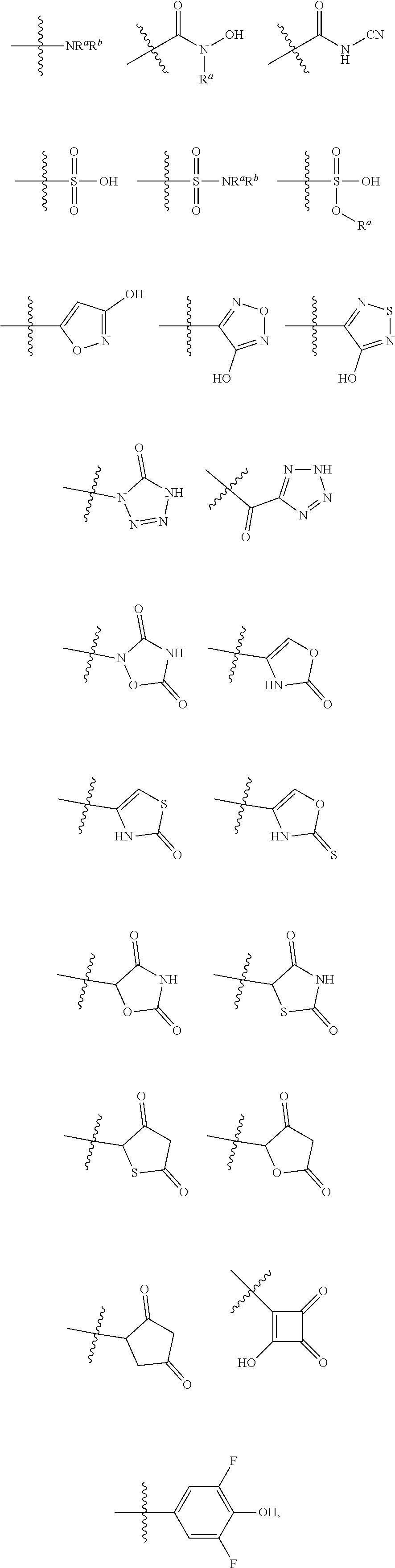

or is selected from

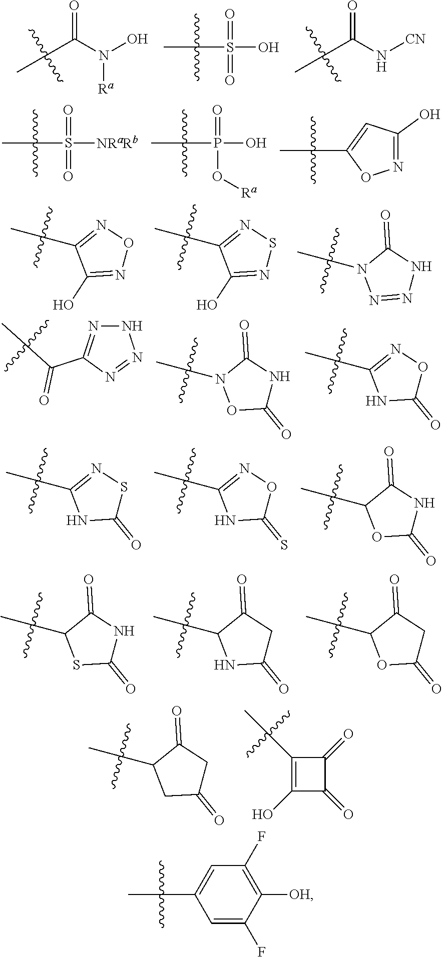

and
each other variable is independently as described herein.

In some embodiments, a provided compound is a compound of formula XII or a salt thereof:

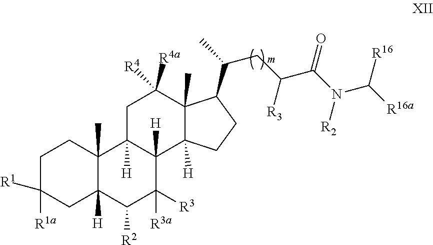

wherein:
   R¹⁶ is selected from:

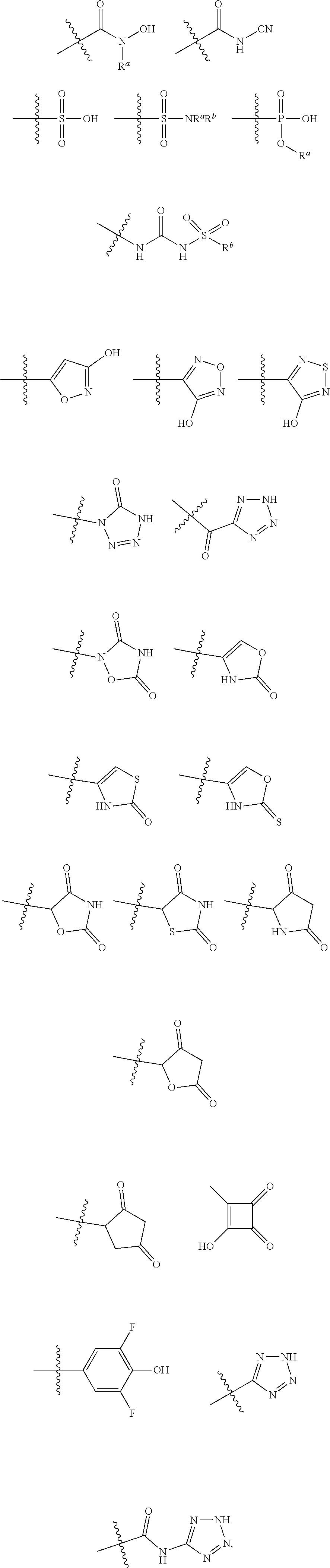

or R¹⁶ is selected from:

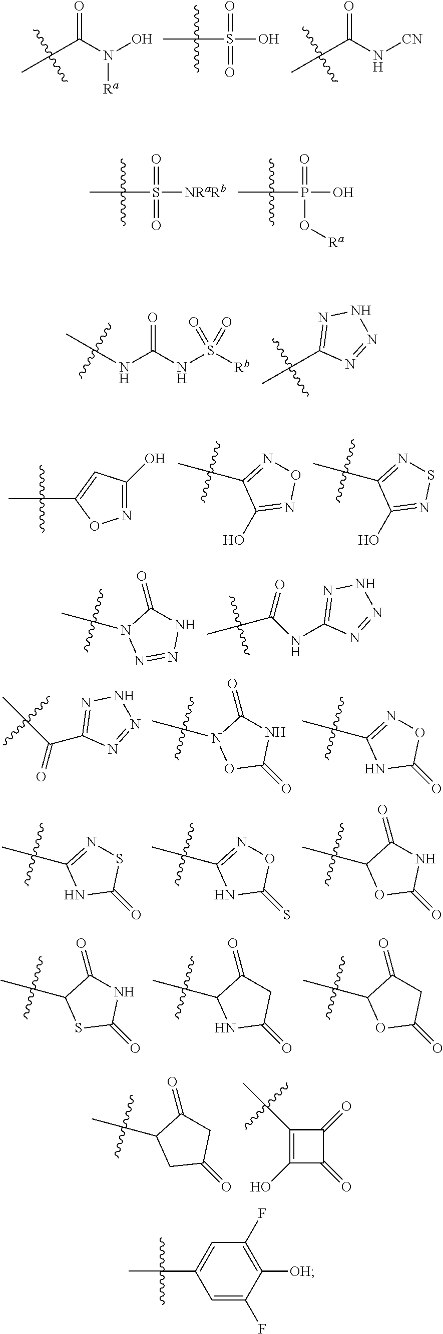

and
each other variable is independently as described herein.

In some embodiments, R¹⁶ is selected from:

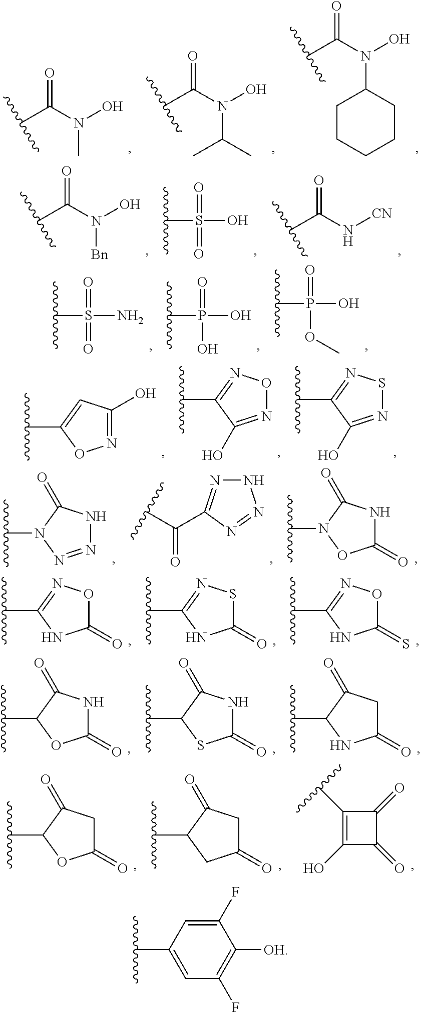

In some embodiments, R¹⁶ is selected from:

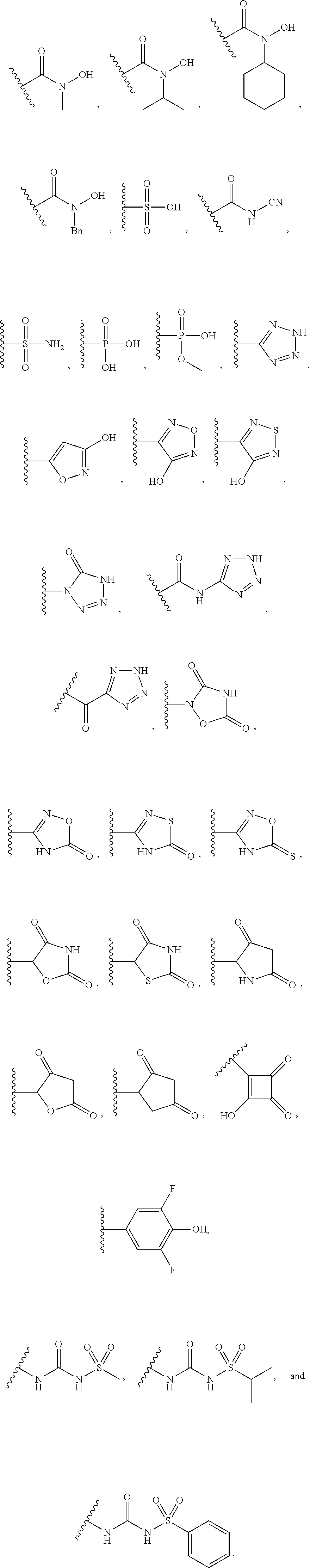

In some embodiments, R¹⁵ or R¹⁶ is —S(O)₂N^{a}R^{b}, wherein R^{a} and R^{b} are —H. In some embodiments, R^{b} is an optionally substituted group selected from C₁₋₄ alkyl, C₁₋₄ haloalkyl, C₂₋₄ alkenyl, phenyl-C₁₋₄ alkyl, C₃₋₆ cycloalkyl, C₃₋₆ cycloalkyl-C₁₋₄ alkyl, and phenyl. In some embodiments, R^{b} is C₁₋₄ alkyl, C₁₋₄ haloalkyl, C₂₋₄ alkenyl, phenyl-C₁₋₄ alkyl, optionally substituted C₃₋₆ cycloalkyl, C₃₋₆ cycloalkyl-C₁₋₄ alkyl, optionally substituted phenyl and halogen. In some embodiments, R^{b} is R₁ as described herein. In some embodiments, R^{b} is methyl, ethyl, butyl, t-butyl, propyl, isopropyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl, 2-methoxyphenyl, or —F. In some embodiments, R is methyl, ethyl, butyl, t-butyl, propyl, isopropyl, benzyl, vinyl, ally, —CF₃, cyclopropyl, 1-methylcyclopropyl, cyclopropylmethyl, phenyl, 4-trifluoromethoxyphenyl, 2-methylphenyl, 2-trifluoromethoxyphenyl, 2-fluorophenyl, 2-naphthyl, 4-methylphenyl, 2-methoxyphenyl, or —F.

In some embodiments, R^{b} is selected from:

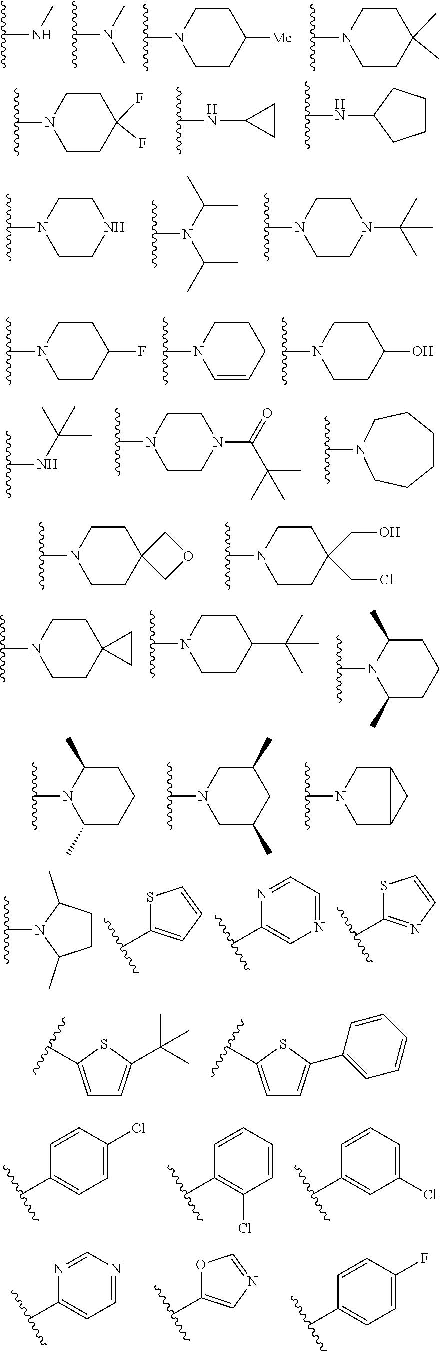

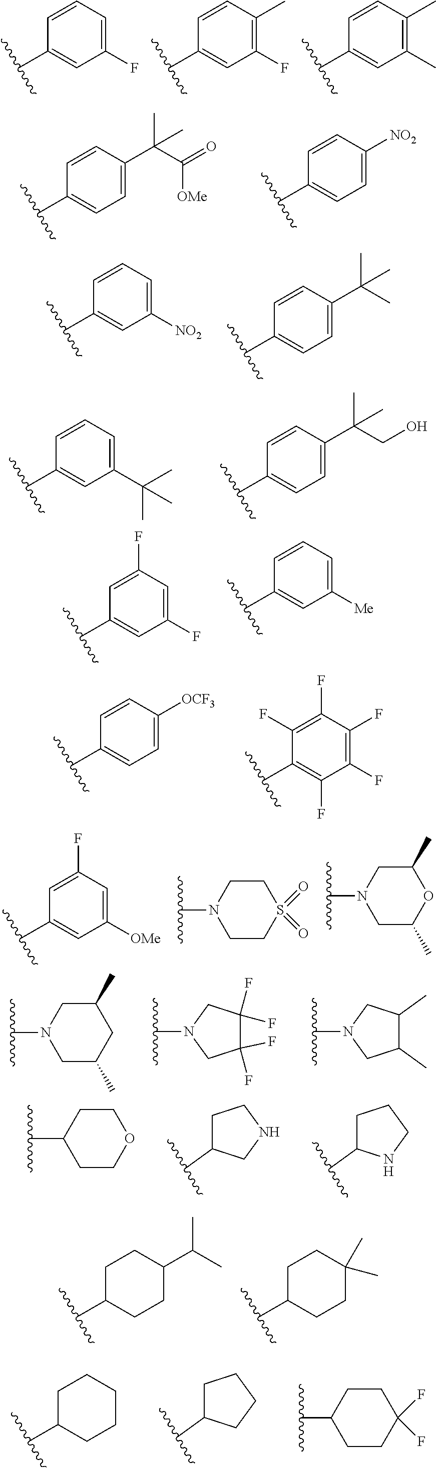

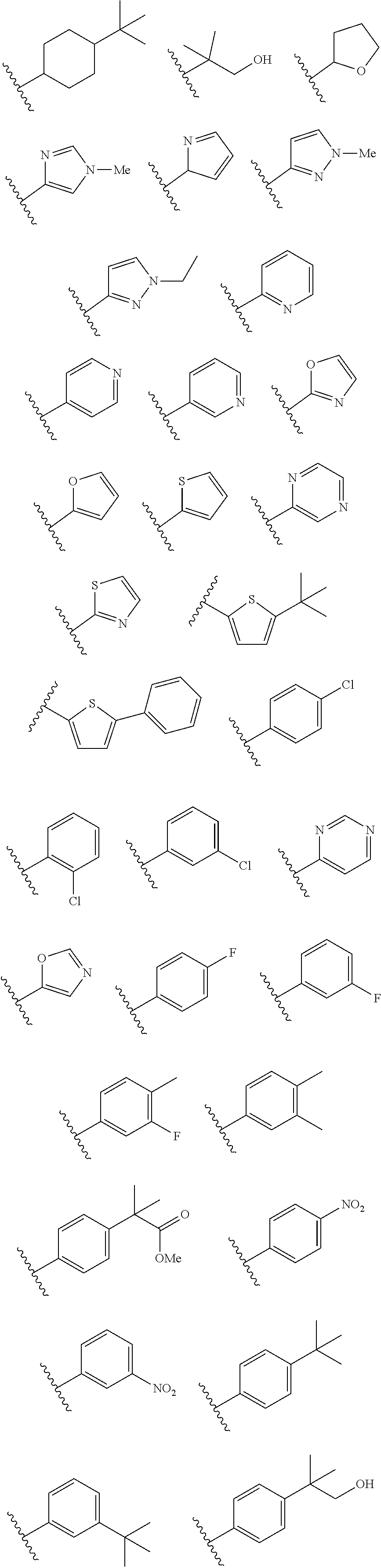

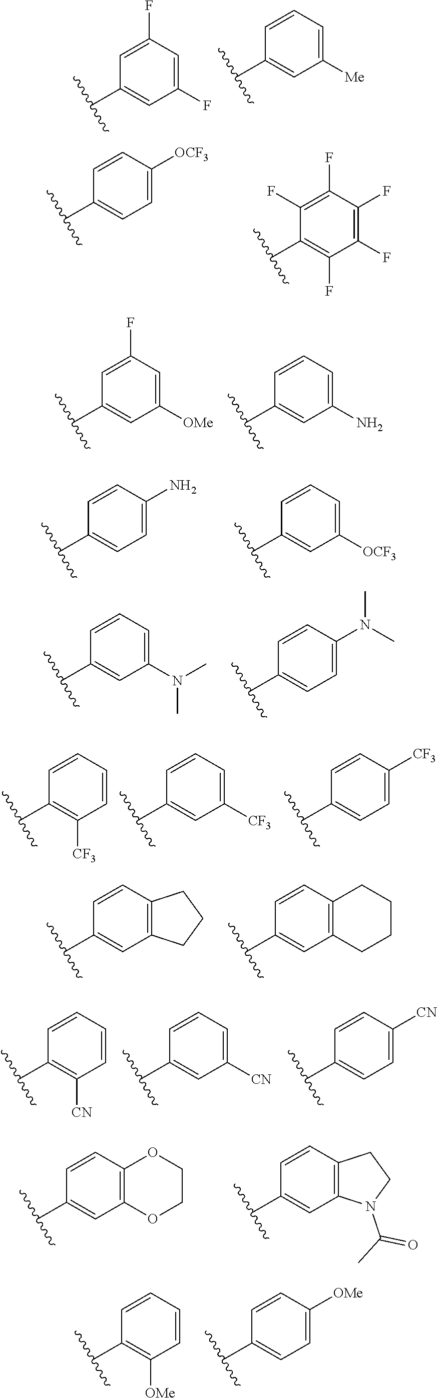

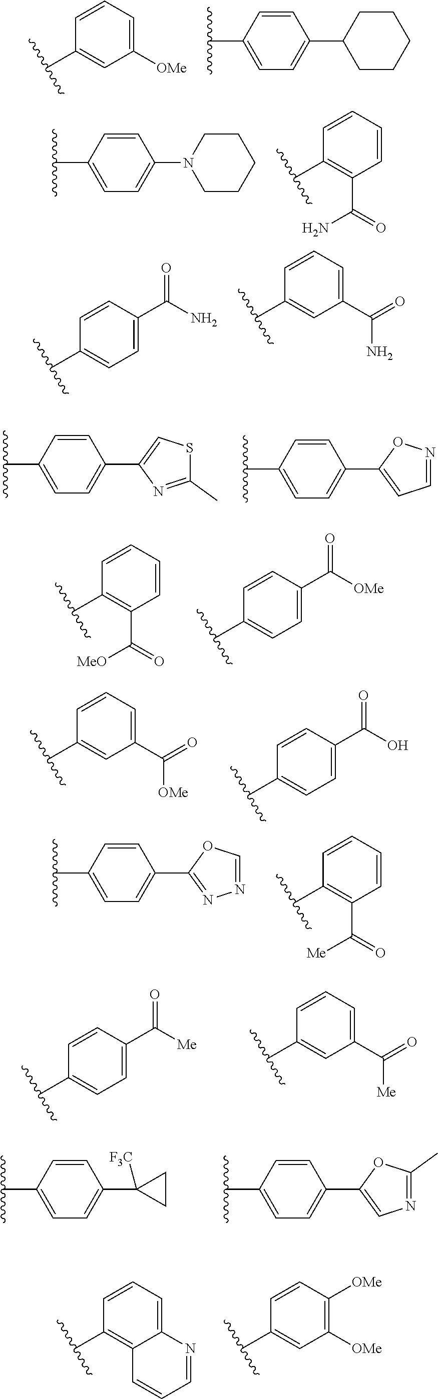

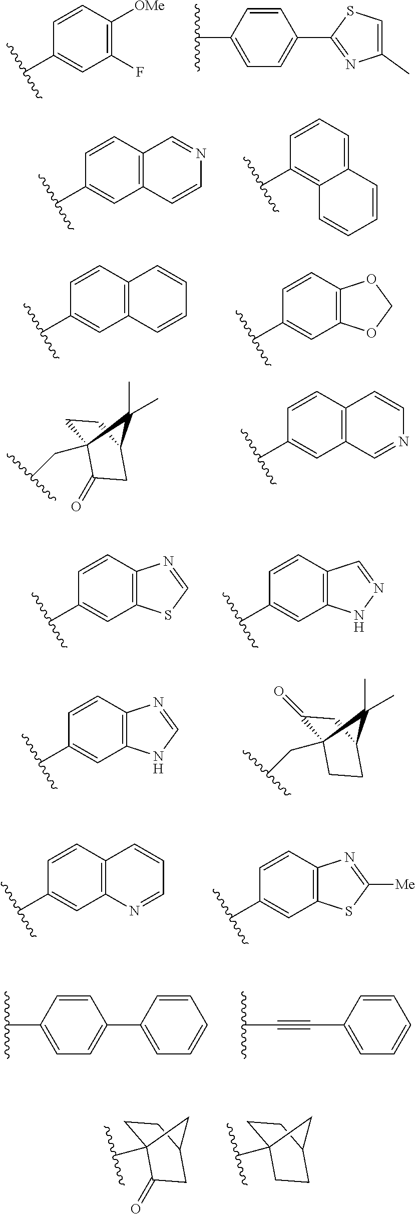

In some embodiments, R₉ is —H. In some embodiments, R₉ is —H and R₁₀ is not —H. In some embodiments, R₁₀ is C₁₋₄ alkyl. In some embodiments, R₁₀ is C₁₋₄ haloalkyl. In some embodiments, R₁₀ is C₂₋₄ alkenyl. In some embodiments, R₁₀ is C₂₋₄ alkynyl. In some embodiments, R₁₀ is phenyl-C₁₋₄ alkyl. In some embodiments, R₁₀ is optionally substituted C₃₋₆ cycloalkyl. In some embodiments, R₁₀ is optionally substituted C₃₋₆ cycloalkyl-C₁₋₄ alkyl. In some embodiments, R₁₀ is 5- or 6-membered heterocyclyl. In some embodiments, R₁₀ is amino. In some embodiments, R₁₀ is optionally substituted phenyl. In some embodiments, R₁₀ is R as described herein. In some embodiments, R₁₀ is optionally substituted C₁₋₆ alkyl. In some embodiments, R₁₀ is methyl. In some embodiments, R₁₀ is ethyl. In some embodiments, R₁₀ is butyl. In some embodiments, R₁₀ is t-butyl. In some embodiments, R₁₀ is propyl. In some embodiments, R₁₀ is isopropyl. In some embodiments, R₁₀ is benzyl. In some embodiments, R₁₀ is vinyl. In some embodiments, R₁₀ is allyl. In some embodiments, R₁₀ is —CF₃. In some embodiments, R₁₀ is cyclopropyl. In some embodiments, R₁₀ is 1-methylcyclopropyl. In some embodiments, R₁₀ is cyclopropylmethyl. In some embodiments, R₁₀ is 1-pyrrolidinyl. In some embodiments, R₁₀ is 1-piperidinyl. In some embodiments, R₁₀ is 4-morpholinyl. In some embodiments, R₁₀ is —NH₂. In some embodiments, R₁₀ is dimethylamino. In some embodiments, R₁₀ is optionally substituted phenyl. In some embodiments, R₁₀ is phenyl. In some embodiments, R₁₀ is 4-trifluoromethoxyphenyl. In some embodiments, R₁₀ is 2-methylphenyl. In some embodiments, R₁₀ is 2-trifluoromethoxyphenyl. In some embodiments, R₁₀ is 2-fluorophenyl. In some embodiments, R₁₀ is 4-tert-butylphenyl. In some embodiments, R₁₀ is 2-naphthyl. In some embodiments, R₁₀ is 4-methylphenyl. In some embodiments, R₁₀ is 2-methoxyphenyl. In some embodiments, R₁₀ is —H.

In some embodiments, m and R¹⁵, or m and R¹⁶, are described as in Tables 1-6 of WO 2016/086169.

Various additional embodiments of R^{a}, R^{b}, R₂, R₃, R₄, R₅, R₇, R⁸, R₉, R₁₀, R¹⁵, R¹⁶, and R^{16a} are described in WO 2016/086169 (e.g., described as Rₐ, R_{b}, R₈, R₂, R₃, R₄, R₆, R₇, R₁₀, R₁₁, R₁, R₁, and R⁹ respectively, in WO 2016/086169), either individually or in combination (e.g., as in compounds).

In some embodiments, a provided compound is a compound selected from compounds in compounds 1-540, Tables 1-6, Schemes 1-8, and Examples 1-13 of WO 2016/086169, wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2016/130809, e.g., a compound of formula (I) or a salt thereof (including stereoisomer, solvate, hydrate or combination thereof) as described in WO 2016/130809, wherein —OR₆ in WO 2016/130809 (in various embodiments, —OR₆ is —OH in WO 2016/130809) is replaced with R¹ or R^{1a} as described herein. In some embodiments, —OR₆ in WO 2016/130809 and the —H that is attached to the same carbon as —OR₆ are replaced with R¹ and R^{1a} as described herein, e.g., in some embodiments, both replaced with —H.

In some embodiments, one of R² and R^{2a} is —H and the other is R₈ as described herein. In some embodiments, one of R³ and R^{3a} is —H and the other is —OR₇ as described herein. In some embodiments, one of R⁴ and R^{4a} is —H and the other is R₄ as described herein. In some embodiments, one of R¹⁴ and R^{14a} is —H and the other is R₅ as described herein. In some embodiments, R⁵ is —H. In some embodiments, R⁶ is —H. In some embodiments, R^{6a} is —H. In some embodiments, R⁷ is —H. In some embodiments, R^{1a} is —H. In some embodiments, R⁸ is —H. In some embodiments, R⁹ is R as described herein. In some embodiments, R¹³ is R as described herein. In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{s0}-L^{s1a}-L^{s1b}-L^{s2}-, wherein each variable is independently as described herein. In some embodiments, L^{s2} is —C(O)—. In some embodiments, L^{s2} is —C(O)N(R′)—. In some embodiments, L^{s2} is —C(O)NH—. In some embodiments, L^{s2} is —C(O)N(R′)C(NR′)—. In some embodiments, L^{s2} is —C(O)NHC(NH)—. In some embodiments, L^{s2} is —C(O)N(R′)C(NR′)N(R′)—. In some embodiments, L^{s2} is —C(O)NHC(NH)NH—. In some embodiments, L¹ is -L^{s0}-L^{s1a}-L^{s1b}-C(O)N(R′)C(NR′)N(R′)—. In some embodiments, L¹ is -L^{s0}-L^{s1a}-L^{s1b}-C(O)NHC(NH)NH—. In some embodiments, L^{s0} is optionally substituted —CH₂—. In some embodiments, L^{s0} is —C(R′)₂—, In some embodiments, L^{s0} is —CHR′—. In some embodiments, L^{s0} is —CH(CH₃)—. In some embodiments, L^{s0} is —(S)—CH(CH₃)—. In some embodiments, L^{s0} is —(R)—CH(CH₃)—. In some embodiments, L^{s1a} is optionally substituted C₁-C₅ alkylene. In some embodiments, L^{s1a} is —(CH₂)m-. In some embodiments, L^{s1b} is optionally substituted —CH₂—. In some embodiments, L^{s1b} is —C(R′)₂—. In some embodiments, L^{s1b} is —CHR′—. In some embodiments, L^{s1b} is —CH(CH₃)—. In some embodiments, L^{s1b} is —(R)—CH(CH₃)—. In some embodiments, L^{s1b} is —(S)—CH(CH₃)—.

In some embodiments, L¹ is —CH(CH₃)—(CH₂)m-CH₂—C(O)—. In some embodiments, L¹ is —CH(CH₃)—(CH₂)m-CH₂—C(O)N(R′)—. In some embodiments, L¹ is —CH(CH₃)—(CH₂)m-CH₂—C(O)NH—. In some embodiments, L¹ is —CH(CH₃)—(CH₂)m-CH₂—C(O)N(R′)—C(NR′)—. In some embodiments, L¹ is —CH(CH₃)—(CH₂)m-CH₂—C(O)NH—C(NH)—. In some embodiments, L¹ is —CH(CH₃)—(CH₂)m-CH₂—C(O)N(R′)—C(NR′)—N(R′)—. In some embodiments, L¹ is —CH(CH₃)—(CH₂)m-CH₂—C(O)NH—C(NH)—NH—. In some embodiments, L¹ is —CH(CH₃)—(CH₂)m-CH(CH₃)—C(O)—. In some embodiments, L¹ is —CH(CH₃)—(CH₂)m-CH(CH₃)—C(O)N(R′)—. In some embodiments, L¹ is —CH(CH₃)—(CH₂)m-CH(CH₃)—C(O)NH—. In some embodiments, L¹ is —CH(CH₃)—(CH₂)m-CH(CH₃)—C(O)N(R′)—C(NR′)—. In some embodiments, L¹ is —CH(CH₃)—(CH₂)m-CH(CH₃)—C(O)NH—C(NH)—. In some embodiments, L¹ is —CH(CH₃)—(CH₂)m-CH(CH₃)—C(O)N(R′)—C(NR′)—N(R′)—. In some embodiments, L¹ is —CH(CH₃)—(CH₂)m-CH(CH₃)—C(O)NH—C(NH)—NH—. In some embodiments, —CH(CH₃)— at the end is S; in some embodiments, it is R. In some embodiments, —CH(CH₃)— in the middle is S; in some embodiments, it is R.

In some embodiments, L¹ is -L^{a}-L^{b}-L^{c}-, wherein L^{a} is L^{s0} as described herein, L^{c} is L^{s1b} as described herein, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{a} is optionally substituted C₁-C₅ alkylene. In some embodiments, L^{s1a} is —(CH₂)m-. In some embodiments, L^{c} is optionally substituted —CH₂—. In some embodiments, L^{c} is —C(R′)₂—. In some embodiments, L^{c} is —CHR′—. In some embodiments, L^{c} is —CH(CH₃)—. In some embodiments, L^{c} is —(R)—CH(CH₃)—. In some embodiments, L^{c} is —(S)—CH(CH₃)—. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, L¹ is —CH(CH₃)(CH₂)mCH₂-, wherein —CH₂— is bonded to R^{L}. In some embodiments, L¹ is (R)—CH(CH₃)(CH₂)mCH₂-, wherein —CH₂— is bonded to R^{L}. In some embodiments, L¹ is (S)—CH(CH₃)(CH₂)mCH₂-, wherein —CH₂— is bonded to R^{L}. In some embodiments, L¹ is (R, R)—CH(CH₃)(CH₂)mCH(CH₃)— which is bonded to R^{L} on its right end. In some embodiments, L¹ is (R, S)—CH(CH₃)(CH₂)mCH(CH₃)— which is bonded to R^{L} on its right end. In some embodiments, L¹ is (S, R)—CH(CH₃)(CH₂)mCH(CH₃)— which is bonded to R^{L} on its right end. In some embodiments, L¹ is (S, S)—CH(CH₃)(CH₂)mCH(CH₃)— which is bonded to R^{L} on its right end.

In some embodiments, R^{L} is —C(O)N(R′)C(NR′)N(R^{s})₂. In some embodiments, the two R′ are taken together with their intervening atoms to form an optionally substituted ring as described herein (e.g., 4-10 (e.g., 4, 5, 6, 7, 8, 9 or 10) membered partially unsaturated or aromatic ring having 0-3 heteroatoms in addition to the intervening atoms). In some embodiments, one R^{s} and the R′ of —C(NR′) are taken together with their intervening atoms to form an optionally substituted ring as described herein (e.g., 4-10 (e.g., 4, 5, 6, 7, 8, 9 or 10) membered partially unsaturated or aromatic ring having 0-3 heteroatoms in addition to the intervening atoms). In some embodiments, one R^{s} and the R′ of —N(R′)— are taken together with their intervening atoms to form an optionally substituted ring as described herein (e.g., 4-10 (e.g., 4, 5, 6, 7, 8, 9 or 10) membered partially unsaturated or aromatic ring having 0-3 heteroatoms in addition to the intervening atoms). In some embodiments, R^{L} is —C(O)NHC(NH)NHR^{s}. In some embodiments, each R^{s} is independently R′.

In some embodiments, a provided compound is a compound of the formula below or a salt thereof:

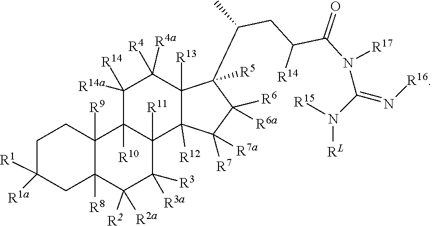

In some embodiments, a provided compound is a compound of formula VIII or a salt thereof, where each variable is independently as described herein.

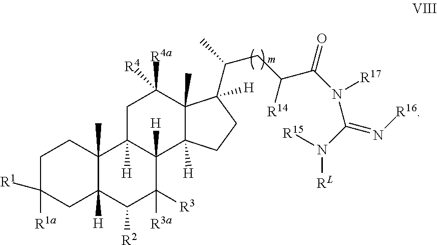

In some embodiments, a provided compound is a compound of a formula selected from formulae VIII-1 to VIII-12, or a salt thereof, where each variable is independently as described herein:

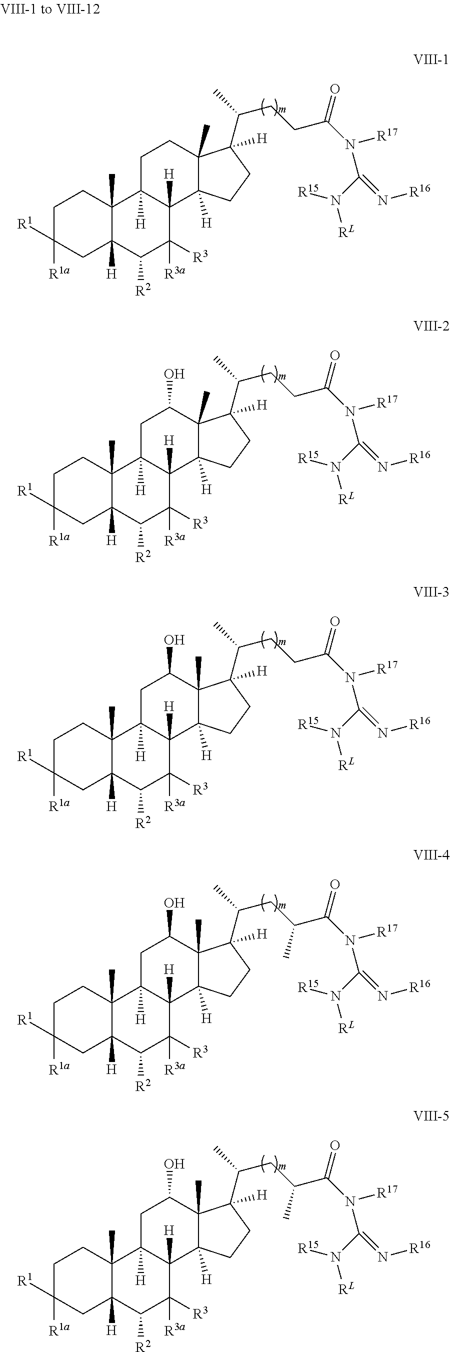

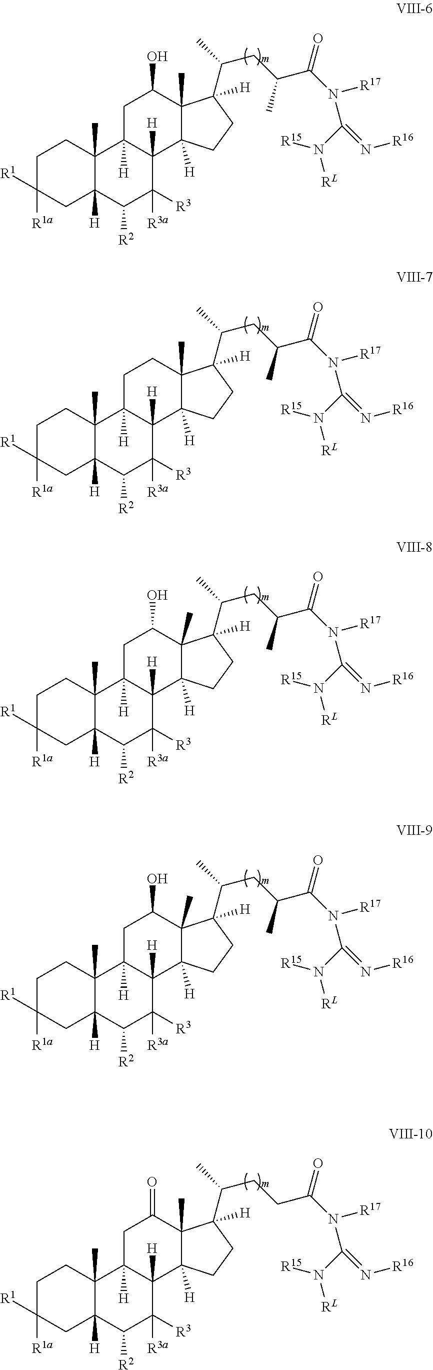

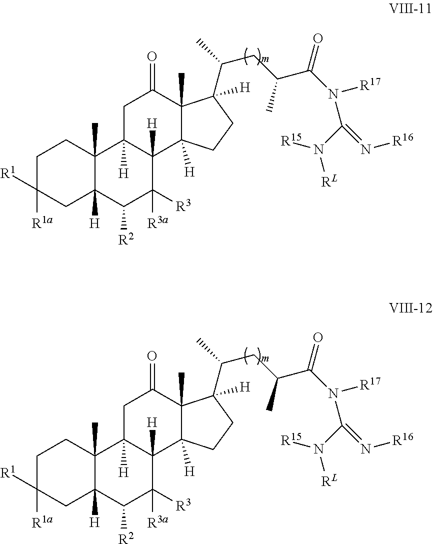

In some embodiments, R^{L} is —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
each of R¹⁵, R¹⁶ and R¹⁷ is independently —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl) (e.g., each is —H); or R¹⁵ and R¹⁷, or R¹⁵ and R¹⁶, or R¹⁶ and R¹⁷, are taken together with their intervening atoms to form an optionally substituted ring (e.g., 4-10 (e.g., 4, 5, 6, 7, 8, 9 or 10) membered partially unsaturated or aromatic ring having 0-3 heteroatoms in addition to the intervening atoms);
R¹⁴ is —H, halogen, or an optionally substituted group selected from C₁₋₈ alkyl, C₂₋₈ alkenyl, C₂₋₈ alkynyl, aryl (e.g., C₆₋₁₄ aryl), arylalkyl (e.g., C₆₋₁₄arylC₁₋₁₂alkyl) (e.g., in some embodiments, R¹⁴ is —H; in some embodiments, R¹⁴ is methyl; etc.); and
m is 0, 1, 2, or 3 (e.g., in some embodiments, m is 0; in some embodiments, m is 1; in some embodiments, m is 2; in some embodiments, m is 3).

In some embodiments, each of R^{1a} and R¹ is —H. In some embodiments, R² is ethyl. In some embodiments, R³ and R^{3a} are —H. In some embodiments, one of R³ and R^{3a} is —H and the other is —OH.

In some embodiments, a provided compound is a compound of formula E-I or a salt thereof:

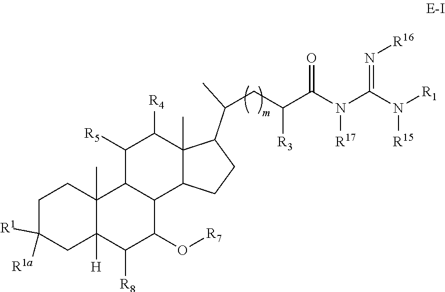

wherein:
   each of R¹ and R^{1a} is independently as described herein;
   R₁ is R₃₀ or R^{s} as described herein;
   R₃₀ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), alkylheteroaryl (e.g., C₁₋₁₂alkyl(5-14 membered heteroaryl having 1-10 heteroatoms)), and heteroarylalkyl (e.g., (5-14 membered heteroaryl having 1-10 heteroatoms)C₁₋₁₂alkyl);
   m is 0, 1, 2, or 3; and
   each of R₃, R⁴, R₅, R₇, R₈, R¹⁵, R¹⁶ and R¹⁷ is independently R^{s} as described herein.

In some embodiments, a provided compound is a compound of formula E-I′ or a salt thereof:

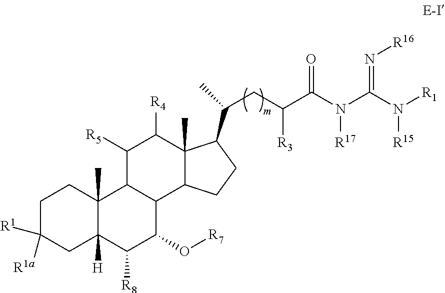

wherein each variable is independently as described herein.

In some embodiments:
each of R¹ and R^{1a} is independently as described herein;
R₁ is —H, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
each of R¹⁵, R¹⁶ and R¹⁷ is independently R₁; preferably R¹⁵, R¹⁶ and R¹⁷ are —H; or R¹⁵ and R¹⁷, or R¹⁵ and R¹⁶, or R¹⁶ and R¹⁷, are taken together with their intervening atoms to form an optionally substituted ring (e.g., 4-10 (e.g., 4, 5, 6, 7, 8, 9 or 10) membered partially unsaturated or aromatic ring having 0-3 heteroatoms in addition to the intervening atoms);
R₃ is —H, halogen, or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H or —OH; more preferably —H;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
Rₛ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably R₈ is ethyl.

In some embodiments, a provided compound is a compound of formula E-IA or a salt thereof:

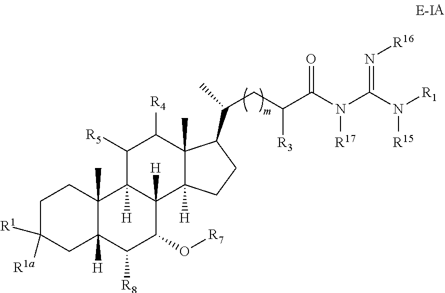

wherein each variable is independently as described herein.

In some embodiments, m is 0, and R^{L} is R₁ as described herein. In some embodiments, m is 1, and R^{L} is R₁ as described herein. In some embodiments, m is 2, and R^{L} is R₁ as described herein. In some embodiments, m is 3, and R^{L} is R₁ as described herein. In some embodiments, R₁ is C₁₋₄ alkyl. In some embodiments, R₁ is C₁₋₄ haloalkyl. In some embodiments, R₁ is C₂₋₄ alkenyl. In some embodiments, R₁ is C₂₋₄ alkynyl. In some embodiments, R₁ is phenyl-C₁₋₄ alkyl. In some embodiments, R₁ is optionally substituted C₃₋₆ cycloalkyl. In some embodiments, R₁ is optionally substituted C₃₋₆ cycloalkyl-C₁₋₄ alkyl. In some embodiments, R₁ is optionally substituted heterocyclyl (e.g., 5- or 6-membered heterocyclyl). In some embodiments, R₁ is optionally substituted 5- or 6-membered heteroaryl. In some embodiments, R₁ is optionally substituted aryl. In some embodiments, R₁ is optionally substituted phenyl. In some embodiments, R₁ is optionally substituted naphthyl. In some embodiments, R₁ is —H. In some embodiments, R₁ is R as described herein. In some embodiments, R₁ is optionally substituted C₁₋₆ alkyl. In some embodiments, R₁ is methyl. In some embodiments, R₁ is ethyl. In some embodiments, R₁ is butyl. In some embodiments, R₁ is t-butyl. In some embodiments, R₁ is propyl. In some embodiments, R₁ is isopropyl. In some embodiments, R₁ is benzyl. In some embodiments, R₁ is vinyl. In some embodiments, R₁ is allyl. In some embodiments, R₁ is —CF₃. In some embodiments, R₁ is cyclopropyl. In some embodiments, R₁ is cyclopentyl. In some embodiments, R₁ is cyclohexyl. In some embodiments, R₁ is 1-methylcyclopropyl. In some embodiments, R₁ is cyclopropylmethyl. In some embodiments, R₁ is optionally substituted phenyl. In some embodiments, R₁ is phenyl. In some embodiments, R₁ is 4-trifluoromethoxyphenyl. In some embodiments, R₁ is 2-methylphenyl. In some embodiments, R₁ is 2-naphthyl. In some embodiments, R₁ is 4-fluorophenyl. In some embodiments, R₁ is 3-fluoro-4-methylphenyl. In some embodiments, R₁ is 4-methylphenyl. In some embodiments, R₁ is 3-pyridinyl. In some embodiments, R₁ is

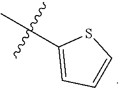 [SMILES: *c1cccs1]

In some embodiments, R₁ is

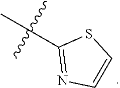 [SMILES: *c1nccs1]

In some embodiments, a provided compound is a compound of formula E-II or a salt thereof:

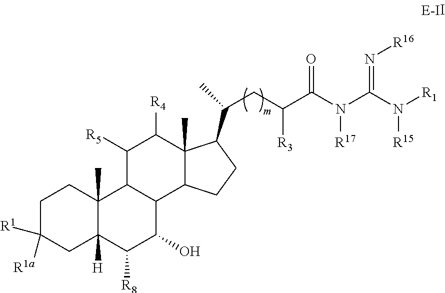

wherein each variable is independently as described herein.

In some embodiments, a provided compound is a compound of formula E-III or a salt thereof:

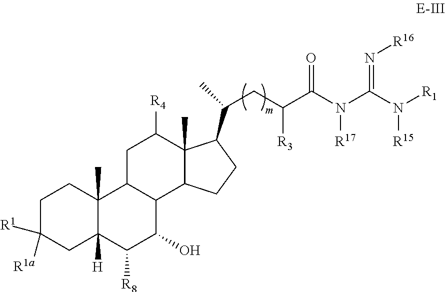

wherein each variable is independently as described herein.

In some embodiments, a provided compound is a compound of formula selected from E-III-1 to E-III-9 or a salt thereof:

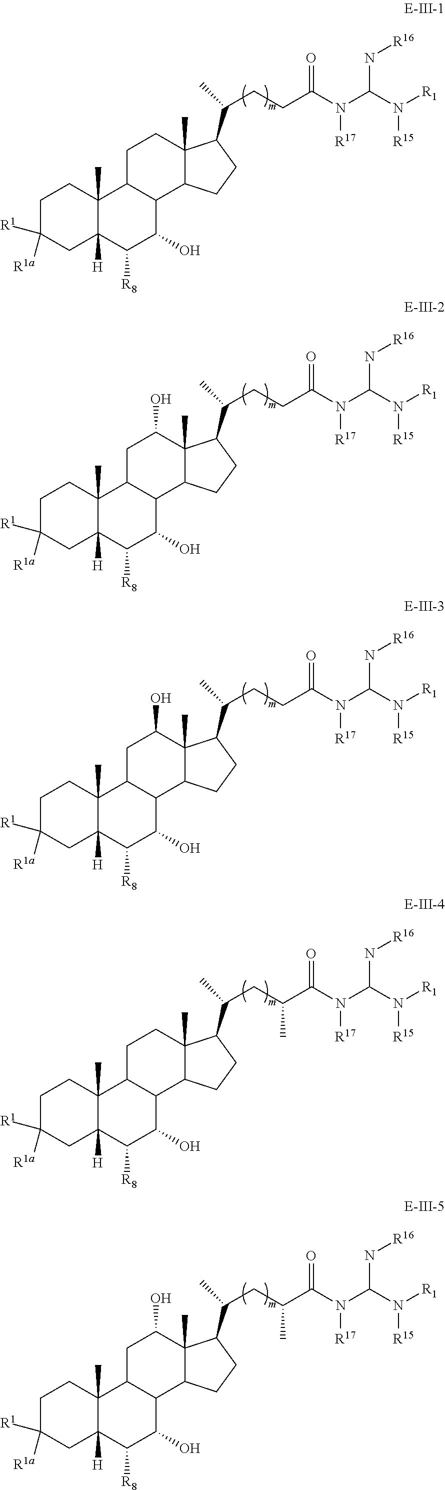

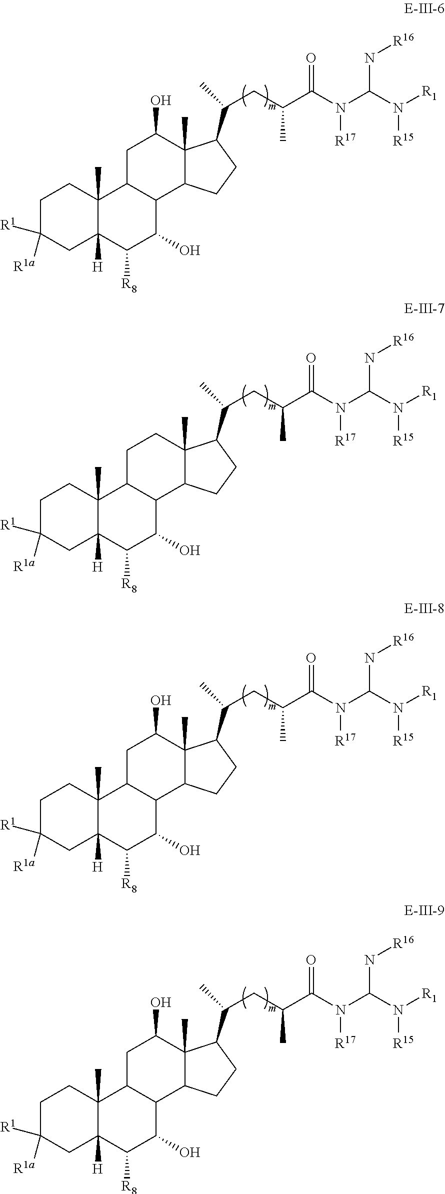

wherein each variable is independently as described herein.

In some embodiments, a provided compound is a compound of formula E-IV or a salt thereof:

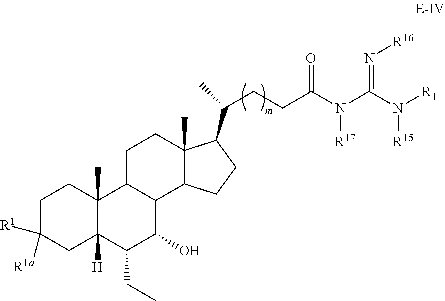

wherein each variable is independently as described herein.

In some embodiments, a provided compound is a compound of formula E-V or a salt thereof:

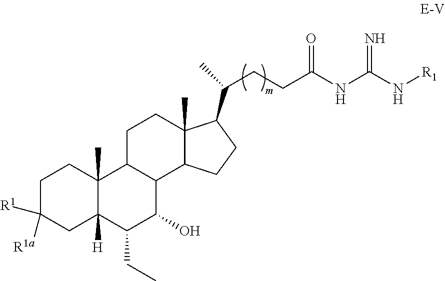

wherein each variable is independently as described herein.

In some embodiments, a provided compound is a compound of formula E-V-I or a salt thereof:

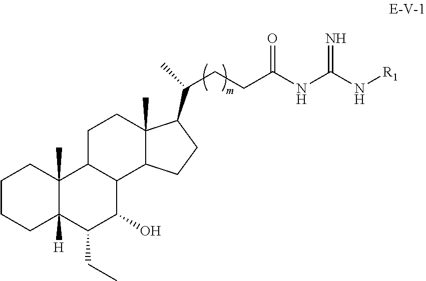

wherein each variable is independently as described herein.

In some embodiments, a provided compound is a compound of formula E-VI or a salt thereof:

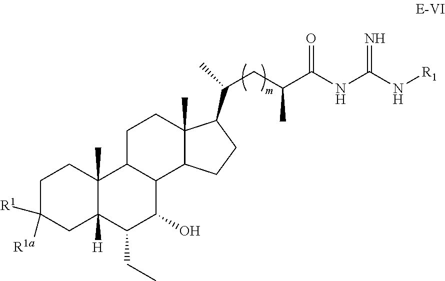

wherein each variable is independently as described herein.

In some embodiments, a provided compound is a compound of formula E-VI-I or a salt thereof:

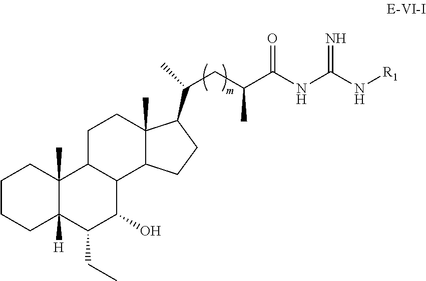

wherein each variable is independently as described herein.

In some embodiments, a provided compound is a compound of formula E-VII or a salt thereof:

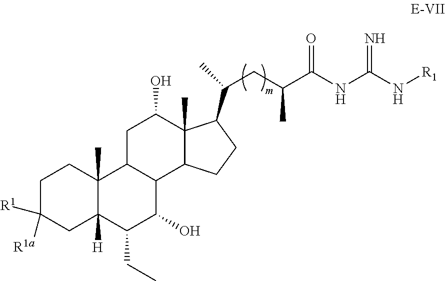

wherein each variable is independently as described herein.

In some embodiments, a provided compound is a compound of formula E-VII-I or a salt

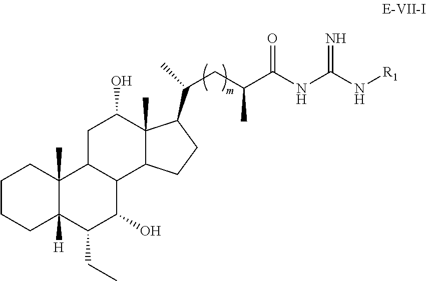

wherein each variable is independently as described herein.

In some embodiments, R¹⁵ and R¹⁶ are taken together with their intervening atoms to form an optionally substituted ring as described herein. In some embodiments, a formed ring is an optionally substituted 5- or 6-membered heterocyclyl ring having 0-1 heteroatoms in addition to the intervening atoms. In some embodiments, a formed ring is an optionally substituted 5- or 6-membered heterocyclyl ring having no heteroatoms in addition to the intervening atoms. For example, as shown in formula E-VIII, in some embodiments, a formed ring is 5-membered.

In some embodiments, a provided compound is a compound of formula E-VIII or a salt thereof:

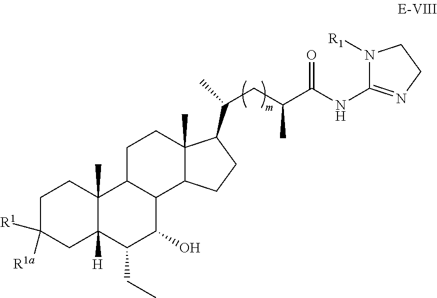

wherein each variable is independently as described herein.

In some embodiments, a provided compound is a compound of formula E-VIII-I or a salt thereof:

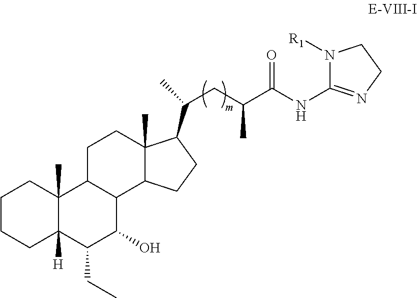

wherein each variable is independently as described herein.

In some embodiments, a provided compound is a compound of formula E-IX or a salt thereof:

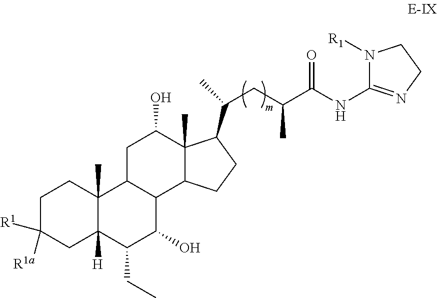

wherein each variable is independently as described herein.

In some embodiments, a provided compound is a compound of formula E-IX-I or a salt thereof:

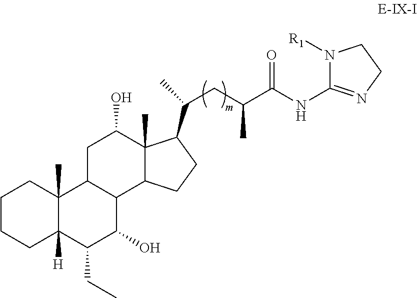

wherein each variable is independently as described herein.

In some embodiments, a provided compound is a compound of formula E-X or a salt thereof:

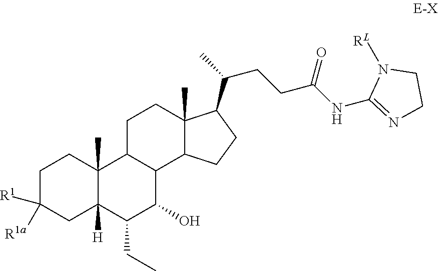

wherein each variable is independently as described herein.

In some embodiments, a provided compound is a compound of formula E-XI or a salt thereof:

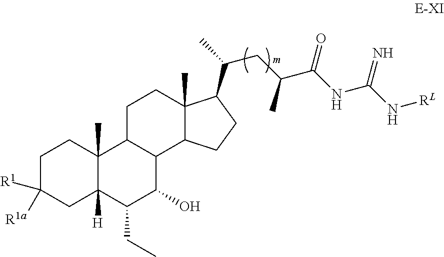

wherein each variable is independently as described herein.

In some embodiments, a provided compound is a compound of formula E-XII or a salt thereof:

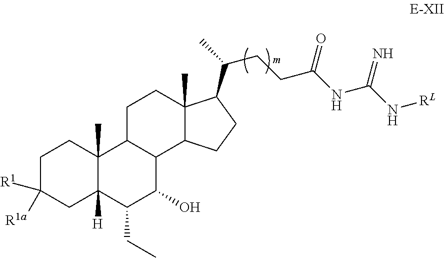

wherein each variable is independently as described herein.

In some embodiments, certain examples of m and R^{L}, and m and R₁, are described in Table 2 below. In some embodiments, m and R₁, or m and R^{L}, are as described in any one of Table 1 to Table 7. In some embodiments, they are as described in Table 1. In some embodiments, they are as described in Table 2. In some embodiments, they are as described in Table 3. In some embodiments, they are as described in Table 4. In some embodiments, they are as described in Table 5. In some embodiments, they are as described in Table 6. In some embodiments, they are as described in Table 7.

In some embodiments, a compound has the structure of formula E-V-I wherein its m and R₁ is described in Table 2 (compounds E-V-I-1 to E-V-I-288, wherein E-V-I-zz has its m and R₁ of entry zz (e.g., compound E-V-I-1 has its m and R₁ of entry 1)) or a salt thereof. In some embodiments, a compound has the structure of formula E-VI-I wherein its m and R₁ is described in Table 2 (compounds E-VI-I-1 to E-VI-I-288, wherein E-VI-I-zz has its m and R₁ of entry zz (e.g., compound E-VI-I-1 has its m and R₁ of entry 1)) or a salt thereof. In some embodiments, a compound has the structure of formula E-VII-I wherein its m and R₁ is described in Table 2 (compounds E-VII-I-1 to E-VII-I-288, wherein E-VII-I-zz has its m and R₁ of entry zz (e.g., compound E-VII-I-1 has its m and R₁ of entry 1)) or a salt thereof. In some embodiments, a compound has the structure of formula E-VIII-I wherein its m and R₁ is described in Table 2 (compounds E-VIII-I-1 to E-VIII-I-288, wherein E-VIII-I-zz has its m and R₁ of entry zz (e.g., compound E-VIII-I-1 has its m and R₁ of entry 1)) or a salt thereof. In some embodiments, a compound has the structure of formula E-IX-I wherein its m and R₁ is described in Table 2 (compounds E-IX-I-1 to E-IX-I-288, wherein E-IX-I-zz has its m and R₁ of entry zz (e.g., compound E-IX-I-1 has its m and R₁ of entry 1)) or a salt thereof.

TABLE 2
Certain examples of m and RL/R1.

| Entry | m | R^{L} |
|---|---|---|
| 1 | 0 | H |
| 2 | 0 | Methyl |
| 3 | 0 | Ethyl |
| 4 | 0 | Isopropyl |
| 5 | 0 | Butyl |
| 6 | 0 | t-Butyl |
| 7 | 0 | Propyl |
| 8 | 0 | Benzyl |
| 9 | 0 | Allyl |
| 10 | 0 | CF₃ |
| 11 | 0 | 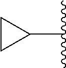 [SMILES: *C1CC1] |
| 12 | 0 | 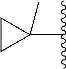 [SMILES: *C1(C)CC1] |
| 13 | 0 | 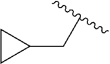 [SMILES: *CC1CC1] |
| 14 | 0 | 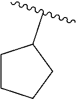 [SMILES: *C1CCCC1] |
| 15 | 0 | 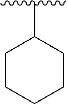 [SMILES: *C1CCCCC1] |
| 16 | 0 | 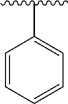 [SMILES: *c1ccccc1] |
| 17 | 0 | 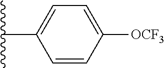 [SMILES: *c1ccc(OC(F)(F)F)cc1] |
| 18 | 0 | 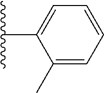 [SMILES: *c1ccccc1C] |
| 19 | 0 | 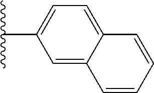 [SMILES: *c1ccc2ccccc2c1] |
| 20 | 0 | 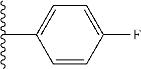 [SMILES: *c1ccc(F)cc1] |
| 21 | 0 | 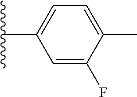 [SMILES: *c1ccc(C)c(F)c1] |
| 22 | 0 | 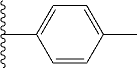 [SMILES: *c1ccc(C)cc1] |
| 23 | 0 | 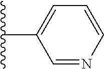 [SMILES: *c1cccnc1] |
| 24 | 0 | 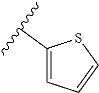 [SMILES: *c1cccs1] |
| 25 | 0 | 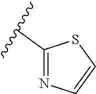 [SMILES: *c1nccs1] |
| 26 | 1 | H |
| 27 | 1 | Methyl |
| 28 | 1 | Ethyl |
| 29 | 1 | Isopropyl |
| 30 | 1 | Butyl |
| 31 | 1 | t-Butyl |
| 32 | 1 | Propyl |
| 33 | 1 | Benzyl |
| 34 | 1 | Allyl |
| 35 | 1 | CF₃ |
| 36 | 1 | 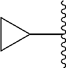 [SMILES: *C1CC1] |
| 37 | 1 | 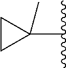 [SMILES: *C1(C)CC1] |
| 38 | 1 | 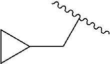 [SMILES: *CC1CC1] |
| 39 | 1 | 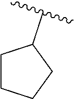 [SMILES: *C1CCCC1] |
| 40 | 1 | 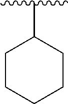 [SMILES: *C1CCCCC1] |
| 41 | 1 | 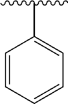 [SMILES: *c1ccccc1] |
| 42 | 1 | 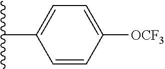 [SMILES: *c1ccc(OC(F)(F)F)cc1] |
| 43 | 1 | 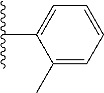 [SMILES: *c1ccccc1C] |
| 44 | 1 | 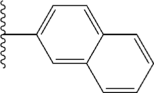 [SMILES: *c1ccc2ccccc2c1] |
| 45 | 1 | 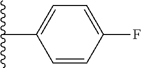 [SMILES: *c1ccc(F)cc1] |
| 46 | 1 | 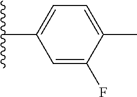 [SMILES: *c1ccc(C)c(F)c1] |
| 47 | 1 | 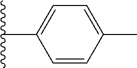 [SMILES: *c1ccc(C)cc1] |
| 48 | 1 | 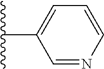 [SMILES: *c1cccnc1] |
| 49 | 1 | 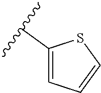 [SMILES: *c1cccs1] |
| 50 | 1 | 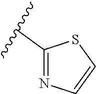 [SMILES: *c1nccs1] |
| 51 | 2 | H |
| 52 | 2 | Methyl |
| 53 | 2 | Ethyl |
| 54 | 2 | Isopropyl |
| 55 | 2 | Butyl |
| 56 | 2 | t-Butyl |
| 57 | 2 | Propyl |
| 58 | 2 | Benzyl |
| 59 | 2 | Allyl |
| 60 | 2 | CF₃ |
| 61 | 2 | 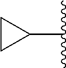 [SMILES: *C1CC1] |
| 62 | 2 | 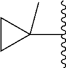 [SMILES: *C1(C)CC1] |
| 63 | 2 | 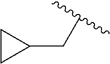 [SMILES: *CC1CC1] |
| 64 | 2 | 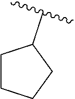 [SMILES: *C1CCCC1] |
| 65 | 2 | 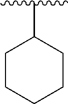 [SMILES: *C1CCCCC1] |
| 66 | 2 | 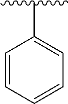 [SMILES: *c1ccccc1] |
| 67 | 2 | 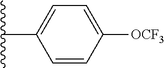 [SMILES: *c1ccc(OC(F)(F)F)cc1] |
| 68 | 2 | 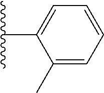 [SMILES: *c1ccccc1C] |
| 69 | 2 | 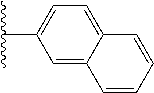 [SMILES: *c1ccc2ccccc2c1] |
| 70 | 2 | 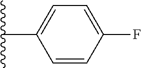 [SMILES: *c1ccc(F)cc1] |
| 71 | 2 | 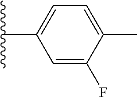 [SMILES: *c1ccc(C)c(F)c1] |
| 72 | 2 | 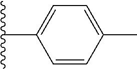 [SMILES: *c1ccc(C)cc1] |
| 73 | 2 | 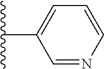 [SMILES: *c1cccnc1] |
| 74 | 2 | 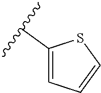 [SMILES: *c1cccs1] |
| 75 | 2 | 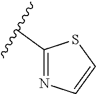 [SMILES: *c1nccs1] |
| 76 | 0 | 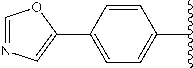 [SMILES: *c1ccc(-c2cnco2)cc1] |
| 77 | 0 | 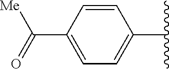 [SMILES: *c1ccc(C(C)=O)cc1] |
| 78 | 0 | 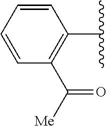 [SMILES: *c1ccccc1C(C)=O] |
| 79 | 0 | 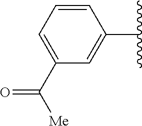 [SMILES: *c1cccc(C(C)=O)c1] |
| 80 | 0 | 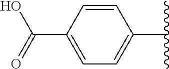 [SMILES: *c1ccc(C(=O)O)cc1] |
| 81 | 0 | 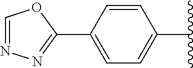 [SMILES: *c1ccc(-c2nnco2)cc1] |
| 82 | 0 | 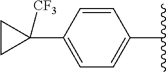 [SMILES: *c1ccc(C2(C(F)(F)F)CC2)cc1] |
| 83 | 0 | 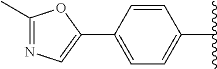 [SMILES: *c1ccc(-c2cnc(C)o2)cc1] |
| 84 | 0 | 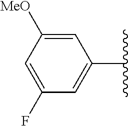 [SMILES: *c1cc(F)cc(OC)c1] |
| 85 | 0 | 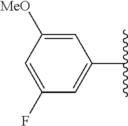 [SMILES: *c1cc(F)cc(OC)c1] |
| 86 | 0 | 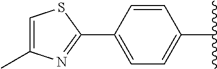 [SMILES: *c1ccc(-c2nc(C)cs2)cc1] |
| 87 | 0 | 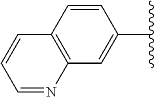 [SMILES: *c1ccc2cccnc2c1] |
| 88 | 0 | 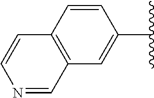 [SMILES: *c1ccc2ccncc2c1] |
| 89 | 0 | 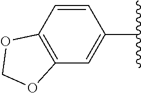 [SMILES: *c1ccc2c(c1)OCO2] |
| 90 | 0 | 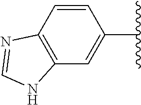 [SMILES: *c1ccc2nc[nH]c2c1] |
| 91 | 0 | 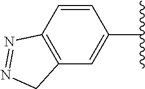 [SMILES: *c1ccc2c(c1)CN=N2] |
| 92 | 0 | 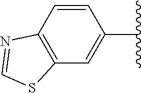 [SMILES: *c1ccc2ncsc2c1] |
| 93 | 0 | 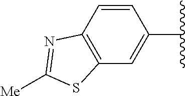 [SMILES: *c1ccc2nc(C)sc2c1] |
| 94 | 0 | 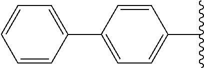 [SMILES: *c1ccc(-c2ccccc2)cc1] |
| 95 | 0 | 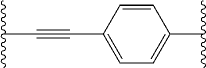 [SMILES: *C#Cc1ccc(*)cc1] |
| 96 | 0 | 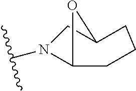 [SMILES: *N1CC2CCCC1O2] |
| 97 | 0 | 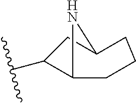 [SMILES: *C1CC2CCCC1N2] |
| 98 | 0 | 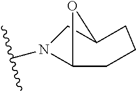 [SMILES: *N1CC2CCCC1O2] |
| 99 | 0 | 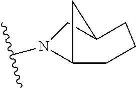 [SMILES: *N1CC2CCCC1C2] |
| 100 | 0 | 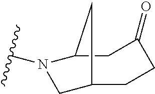 [SMILES: *N1CC2CCC(=O)CC1C2] |
| 101 | 0 | 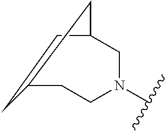 [SMILES: *N1CCC2CCC(C2)C1] |
| 102 | 0 | 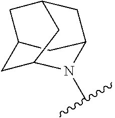 [SMILES: *N1C2CC3CC(C2)CC1C3] |
| 103 | 0 | 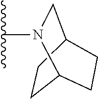 [SMILES: *N1CC2CCC1CC2] |
| 104 | 0 | 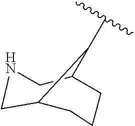 [SMILES: *C1C2CCCC1CNC2] |
| 105 | 0 | 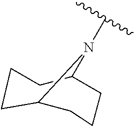 [SMILES: *N1C2CCCC1CCC2] |
| 106 | 0 | 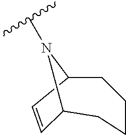 [SMILES: *N1C2C=CC1CCCC2] |
| 107 | 0 | 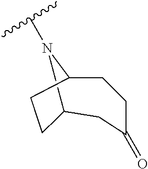 [SMILES: *N1C2CCC(=O)CC1CC2] |
| 108 | 0 | 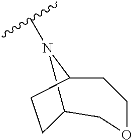 [SMILES: *N1C2CCOCC1CC2] |
| 109 | 0 | 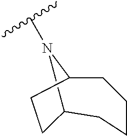 [SMILES: *N1C2CCCCC1CC2] |
| 110 | 0 | 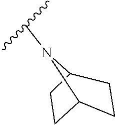 [SMILES: *N1C2CCC1CC2] |
| 111 | 0 | 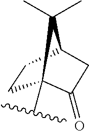 |
| 112 | 1 | 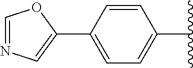 [SMILES: *c1ccc(-c2cnco2)cc1] |
| 113 | 1 | 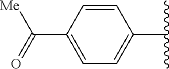 [SMILES: *c1ccc(C(C)=O)cc1] |
| 114 | 1 | 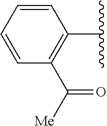 [SMILES: *c1ccccc1C(C)=O] |
| 115 | 1 | 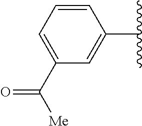 [SMILES: *c1cccc(C(C)=O)c1] |
| 116 | 1 | 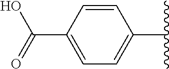 [SMILES: *c1ccc(C(=O)O)cc1] |
| 117 | 1 | 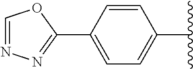 [SMILES: *c1ccc(-c2nnco2)cc1] |
| 118 | 1 | 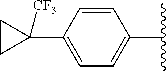 [SMILES: *c1ccc(C2(C(F)(F)F)CC2)cc1] |
| 119 | 1 | 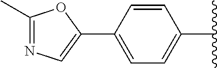 [SMILES: *c1ccc(-c2cnc(C)o2)cc1] |
| 120 | 1 | 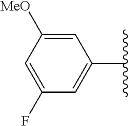 [SMILES: *c1cc(F)cc(OC)c1] |
| 121 | 1 | 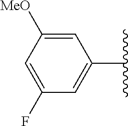 [SMILES: *c1cc(F)cc(OC)c1] |
| 122 | 1 | 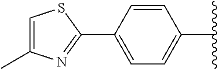 [SMILES: *c1ccc(-c2nc(C)cs2)cc1] |
| 123 | 1 | 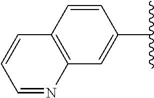 [SMILES: *c1ccc2cccnc2c1] |
| 124 | 1 | 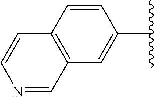 [SMILES: *c1ccc2ccncc2c1] |
| 125 | 1 | 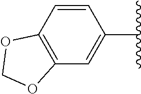 [SMILES: *c1ccc2c(c1)OCO2] |
| 126 | 1 | 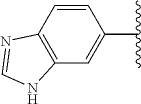 [SMILES: *c1ccc2nc[nH]c2c1] |
| 127 | 1 | 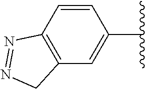 [SMILES: *c1ccc2c(c1)CN=N2] |
| 128 | 1 | 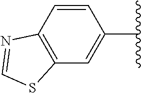 [SMILES: *c1ccc2ncsc2c1] |
| 129 | 1 | 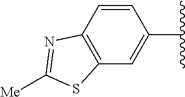 [SMILES: *c1ccc2nc(C)sc2c1] |
| 130 | 1 | 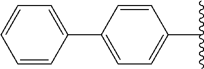 [SMILES: *c1ccc(-c2ccccc2)cc1] |
| 131 | 1 | 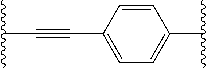 [SMILES: *C#Cc1ccc(*)cc1] |
| 132 | 1 | 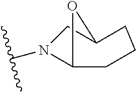 [SMILES: *N1CC2CCCC1O2] |
| 133 | 1 | 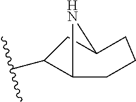 [SMILES: *C1CC2CCCC1N2] |
| 134 | 1 | 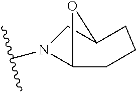 [SMILES: *N1CC2CCCC1O2] |
| 135 | 1 | 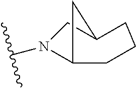 [SMILES: *N1CC2CCCC1C2] |
| 136 | 1 | 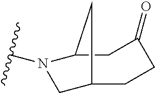 [SMILES: *N1CC2CCC(=O)CC1C2] |
| 137 | 1 | 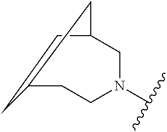 [SMILES: *N1CCC2CCC(C2)C1] |
| 138 | 1 | 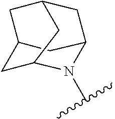 [SMILES: *N1C2CC3CC(C2)CC1C3] |
| 139 | 1 | 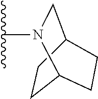 [SMILES: *N1CC2CCC1CC2] |
| 140 | 1 | 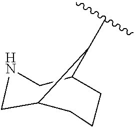 [SMILES: *C1C2CCCC1CNC2] |
| 141 | 1 | 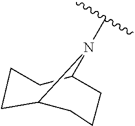 [SMILES: *N1C2CCCC1CCC2] |
| 142 | 1 | 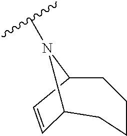 [SMILES: *N1C2C=CC1CCCC2] |
| 143 | 1 | 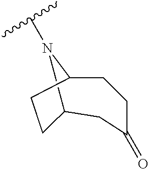 [SMILES: *N1C2CCC(=O)CC1CC2] |
| 144 | 1 | 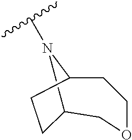 [SMILES: *N1C2CCOCC1CC2] |
| 145 | 1 | 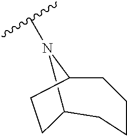 [SMILES: *N1C2CCCCC1CC2] |
| 146 | 1 | 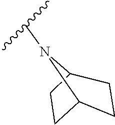 [SMILES: *N1C2CCC1CC2] |
| 147 | 1 | 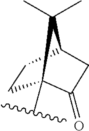 |
| 148 | 2 | 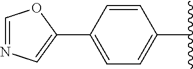 [SMILES: *c1ccc(-c2cnco2)cc1] |
| 149 | 2 | 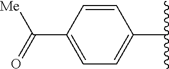 [SMILES: *c1ccc(C(C)=O)cc1] |
| 150 | 2 | 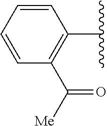 [SMILES: *c1ccccc1C(C)=O] |
| 151 | 2 | 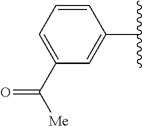 [SMILES: *c1cccc(C(C)=O)c1] |
| 152 | 2 | 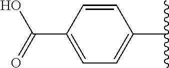 [SMILES: *c1ccc(C(=O)O)cc1] |
| 153 | 2 | 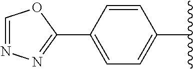 [SMILES: *c1ccc(-c2nnco2)cc1] |
| 154 | 2 | 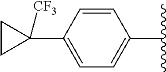 [SMILES: *c1ccc(C2(C(F)(F)F)CC2)cc1] |
| 155 | 2 | 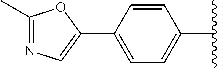 [SMILES: *c1ccc(-c2cnc(C)o2)cc1] |
| 156 | 2 | 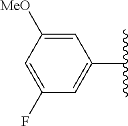 [SMILES: *c1cc(F)cc(OC)c1] |
| 157 | 2 | 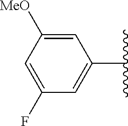 [SMILES: *c1cc(F)cc(OC)c1] |
| 158 | 2 | 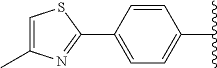 [SMILES: *c1ccc(-c2nc(C)cs2)cc1] |
| 159 | 2 | 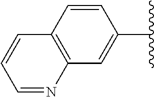 [SMILES: *c1ccc2cccnc2c1] |
| 160 | 2 | 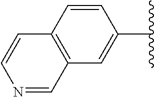 [SMILES: *c1ccc2ccncc2c1] |
| 161 | 2 | 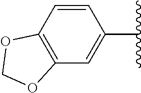 [SMILES: *c1ccc2c(c1)OCO2] |
| 162 | 2 | 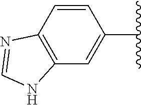 [SMILES: *c1ccc2nc[nH]c2c1] |
| 163 | 2 | 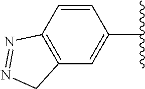 [SMILES: *c1ccc2c(c1)CN=N2] |
| 164 | 2 | 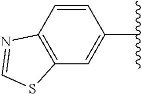 [SMILES: *c1ccc2ncsc2c1] |
| 165 | 2 | 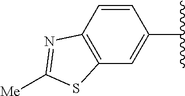 [SMILES: *c1ccc2nc(C)sc2c1] |
| 166 | 2 | 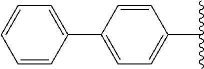 [SMILES: *c1ccc(-c2ccccc2)cc1] |
| 167 | 2 | 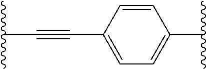 [SMILES: *C#Cc1ccc(*)cc1] |
| 168 | 2 | 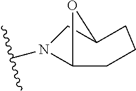 [SMILES: *N1CC2CCCC1O2] |
| 169 | 2 | 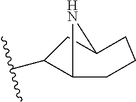 [SMILES: *C1CC2CCCC1N2] |
| 170 | 2 | 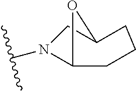 [SMILES: *N1CC2CCCC1O2] |
| 171 | 2 | 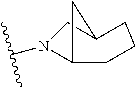 [SMILES: *N1CC2CCCC1C2] |
| 172 | 2 | 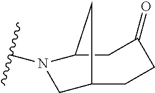 [SMILES: *N1CC2CCC(=O)CC1C2] |
| 173 | 2 | 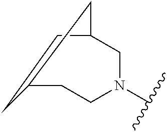 [SMILES: *N1CCC2CCC(C2)C1] |
| 174 | 2 | 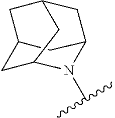 [SMILES: *N1C2CC3CC(C2)CC1C3] |
| 175 | 2 | 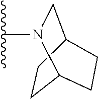 [SMILES: *N1CC2CCC1CC2] |
| 176 | 2 | 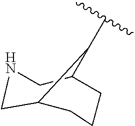 [SMILES: *C1C2CCCC1CNC2] |
| 177 | 2 | 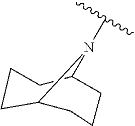 [SMILES: *N1C2CCCC1CCC2] |
| 178 | 2 | 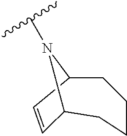 [SMILES: *N1C2C=CC1CCCC2] |
| 179 | 2 | 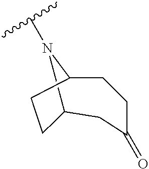 [SMILES: *N1C2CCC(=O)CC1CC2] |
| 180 | 2 | 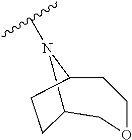 [SMILES: *N1C2CCOCC1CC2] |
| 181 | 2 | 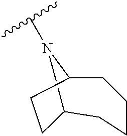 [SMILES: *N1C2CCCCC1CC2] |
| 182 | 2 | 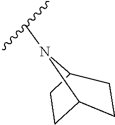 [SMILES: *N1C2CCC1CC2] |
| 183 | 2 | 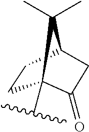 |
| 184 | 0 | 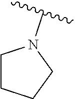 [SMILES: *N1CCCC1] |
| 185 | 0 | 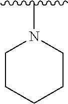 [SMILES: *N1CCCCC1] |
| 186 | 0 | 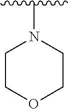 [SMILES: *N1CCOCC1] |
| 187 | 0 | 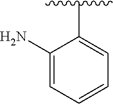 [SMILES: *c1ccccc1N] |
| 188 | 0 | 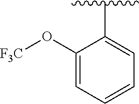 [SMILES: *c1ccccc1OC(F)(F)F] |
| 189 | 0 | 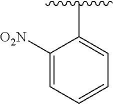 [SMILES: *c1ccccc1[N+](=O)[O-]] |
| 190 | 0 | F |
| 191 | 0 | 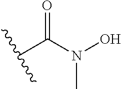 [SMILES: *C(=O)N(C)O] |
| 192 | 0 | 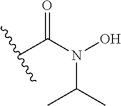 [SMILES: *C(=O)N(O)C(C)C] |
| 193 | 0 | 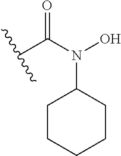 [SMILES: *C(=O)N(O)C1CCCCC1] |
| 194 | 0 | 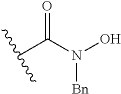 [SMILES: *C(=O)N(O)Cc1ccccc1] |
| 195 | 0 | 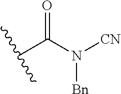 [SMILES: *C(=O)N(C#N)Cc1ccccc1] |
| 196 | 0 | 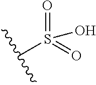 [SMILES: *S(=O)(=O)O] |
| 197 | 0 | 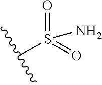 [SMILES: *S(N)(=O)=O] |
| 198 | 0 | 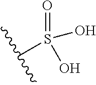 |
| 199 | 0 | 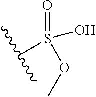 |
| 200 | 0 | 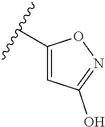 [SMILES: *c1cc(O)no1] |
| 201 | 0 | 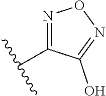 [SMILES: *c1nonc1O] |
| 202 | 0 | 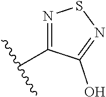 [SMILES: *c1nsnc1O] |
| 203 | 0 | 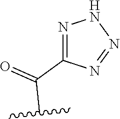 [SMILES: *C(=O)c1nn[nH]n1] |
| 204 | 0 | 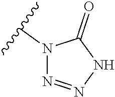 [SMILES: *n1nn[nH]c1=O] |
| 205 | 0 | 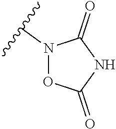 [SMILES: *n1oc(=O)[nH]c1=O] |
| 206 | 0 | 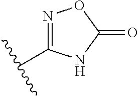 [SMILES: *c1noc(=O)[nH]1] |
| 207 | 0 | 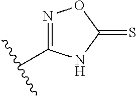 [SMILES: *c1noc(=S)[nH]1] |
| 208 | 0 | 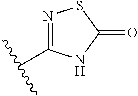 [SMILES: *c1nsc(=O)[nH]1] |
| 209 | 0 | 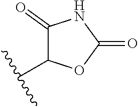 [SMILES: *C1OC(=O)NC1=O] |
| 210 | 0 | 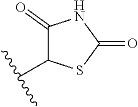 [SMILES: *C1SC(=O)NC1=O] |
| 211 | 0 | 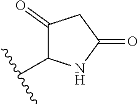 [SMILES: *C1NC(=O)CC1=O] |
| 212 | 0 | 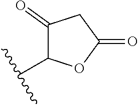 [SMILES: *C1OC(=O)CC1=O] |
| 213 | 0 | 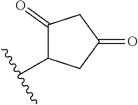 [SMILES: *C1CC(=O)CC1=O] |
| 214 | 0 | 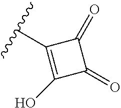 [SMILES: *c1c(O)c(=O)c1=O] |
| 215 | 0 | 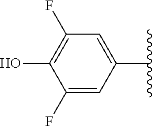 [SMILES: *c1cc(F)c(O)c(F)c1] |
| 216 | 0 | 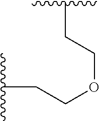 [SMILES: *CCOCC*] |
| 217 | 0 | 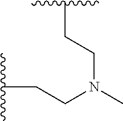 [SMILES: *CCN(C)CC*] |
| 218 | 0 | 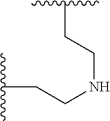 [SMILES: *CCNCC*] |
| 219 | 1 | 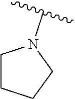 [SMILES: *N1CCCC1] |
| 220 | 1 | 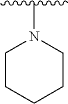 [SMILES: *N1CCCCC1] |
| 221 | 1 | 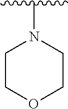 [SMILES: *N1CCOCC1] |
| 222 | 1 | 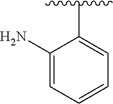 [SMILES: *c1ccccc1N] |
| 223 | 1 | 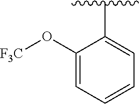 [SMILES: *c1ccccc1OC(F)(F)F] |
| 224 | 1 | 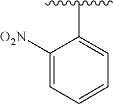 [SMILES: *c1ccccc1[N+](=O)[O-]] |
| 225 | 1 | F |
| 226 | 1 | 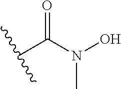 [SMILES: *C(=O)N(C)O] |
| 227 | 1 | 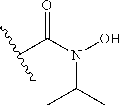 [SMILES: *C(=O)N(O)C(C)C] |
| 228 | 1 | 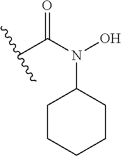 [SMILES: *C(=O)N(O)C1CCCCC1] |
| 229 | 1 | 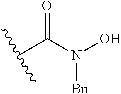 [SMILES: *C(=O)N(O)Cc1ccccc1] |
| 230 | 1 | 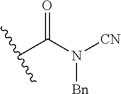 [SMILES: *C(=O)N(C#N)Cc1ccccc1] |
| 231 | 1 | 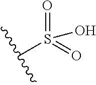 [SMILES: *S(=O)(=O)O] |
| 232 | 1 | 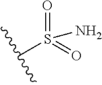 [SMILES: *S(N)(=O)=O] |
| 233 | 1 | 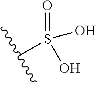 |
| 234 | 1 | 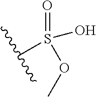 |
| 235 | 1 | 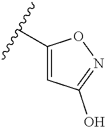 [SMILES: *c1cc(O)no1] |
| 236 | 1 | 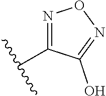 [SMILES: *c1nonc1O] |
| 237 | 1 | 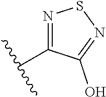 [SMILES: *c1nsnc1O] |
| 238 | 1 | 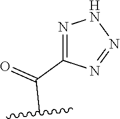 [SMILES: *C(=O)c1nn[nH]n1] |
| 239 | 1 | 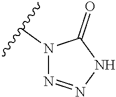 [SMILES: *n1nn[nH]c1=O] |
| 240 | 1 | 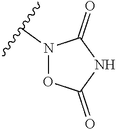 [SMILES: *n1oc(=O)[nH]c1=O] |
| 241 | 1 | 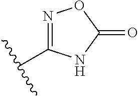 [SMILES: *c1noc(=O)[nH]1] |
| 242 | 1 | 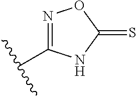 [SMILES: *c1noc(=S)[nH]1] |
| 243 | 1 | 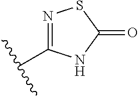 [SMILES: *c1nsc(=O)[nH]1] |
| 244 | 1 | 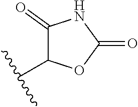 [SMILES: *C1OC(=O)NC1=O] |
| 245 | 1 | 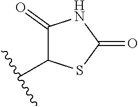 [SMILES: *C1SC(=O)NC1=O] |
| 246 | 1 | 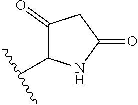 [SMILES: *C1NC(=O)CC1=O] |
| 247 | 1 | 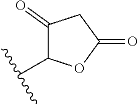 [SMILES: *C1OC(=O)CC1=O] |
| 248 | 1 | 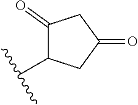 [SMILES: *C1CC(=O)CC1=O] |
| 249 | 1 | 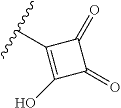 [SMILES: *c1c(O)c(=O)c1=O] |
| 250 | 1 | 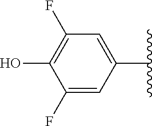 [SMILES: *c1cc(F)c(O)c(F)c1] |
| 251 | 1 | 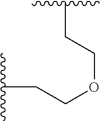 [SMILES: *CCOCC*] |
| 252 | 1 | 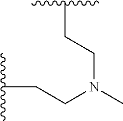 [SMILES: *CCN(C)CC*] |
| 253 | 1 | 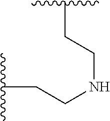 [SMILES: *CCNCC*] |
| 254 | 2 | 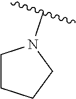 [SMILES: *N1CCCC1] |
| 255 | 2 | 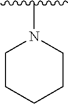 [SMILES: *N1CCCCC1] |
| 256 | 2 | 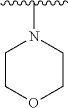 [SMILES: *N1CCOCC1] |
| 257 | 2 | 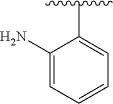 [SMILES: *c1ccccc1N] |
| 258 | 2 | 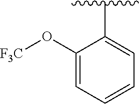 [SMILES: *c1ccccc1OC(F)(F)F] |
| 259 | 2 | 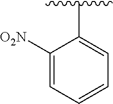 [SMILES: *c1ccccc1[N+](=O)[O-]] |
| 260 | 2 | F |
| 261 | 2 | 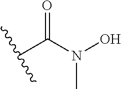 [SMILES: *C(=O)N(C)O] |
| 262 | 2 | 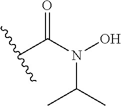 [SMILES: *C(=O)N(O)C(C)C] |
| 263 | 2 | 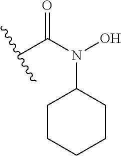 [SMILES: *C(=O)N(O)C1CCCCC1] |
| 264 | 2 | 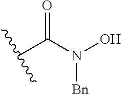 [SMILES: *C(=O)N(O)Cc1ccccc1] |
| 265 | 2 | 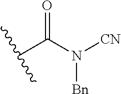 [SMILES: *C(=O)N(C#N)Cc1ccccc1] |
| 266 | 2 | 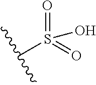 [SMILES: *S(=O)(=O)O] |
| 267 | 2 | 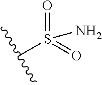 [SMILES: *S(N)(=O)=O] |
| 268 | 2 | 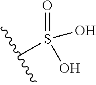 |
| 269 | 2 | 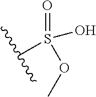 |
| 270 | 2 | 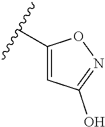 [SMILES: *c1cc(O)no1] |
| 271 | 2 | 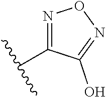 [SMILES: *c1nonc1O] |
| 272 | 2 | 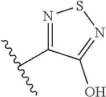 [SMILES: *c1nsnc1O] |
| 273 | 2 | 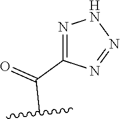 [SMILES: *C(=O)c1nn[nH]n1] |
| 274 | 2 | 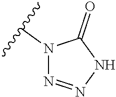 [SMILES: *n1nn[nH]c1=O] |
| 275 | 2 | 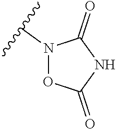 [SMILES: *n1oc(=O)[nH]c1=O] |
| 276 | 2 | 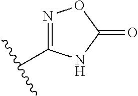 [SMILES: *c1noc(=O)[nH]1] |
| 277 | 2 | 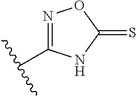 [SMILES: *c1noc(=S)[nH]1] |
| 278 | 2 | 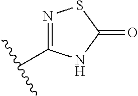 [SMILES: *c1nsc(=O)[nH]1] |
| 279 | 2 | 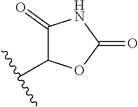 [SMILES: *C1OC(=O)NC1=O] |
| 280 | 2 | 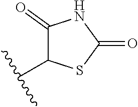 [SMILES: *C1SC(=O)NC1=O] |
| 281 | 2 | 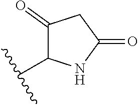 [SMILES: *C1NC(=O)CC1=O] |
| 282 | 2 | 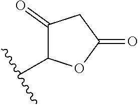 [SMILES: *C1OC(=O)CC1=O] |
| 283 | 2 | 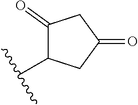 [SMILES: *C1CC(=O)CC1=O] |
| 284 | 2 | 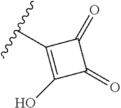 [SMILES: *c1c(O)c(=O)c1=O] |
| 285 | 2 | 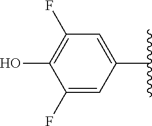 [SMILES: *c1cc(F)c(O)c(F)c1] |
| 286 | 2 | 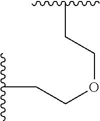 [SMILES: *CCOCC*] |
| 287 | 2 | 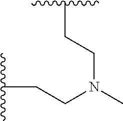 [SMILES: *CCN(C)CC*] |
| 288 | 2 | 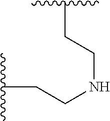 [SMILES: *CCNCC*] |

In some embodiments, a provided compound is a compound selected from compounds 1-300 of WO 2016/130809, wherein 3-OH— and 3-H attached to moiety A is replaced with R¹ and R^{1a}, e.g., in some embodiments, both replaced with —H.

In some embodiments, R₂ is —H. In some embodiments, R₃ is —H. In some embodiments, R₃ is methyl. In some embodiments, R₄ is —H. In some embodiments, R₄ is —OH. In some embodiments, R₅ is —H. In some embodiments, R₆ is —H. In some embodiments, R₇ is —H. In some embodiments, Rₛ is ethyl. In some embodiments, m is 0. In some embodiments, m is 1. In some embodiments, m is 2.

In some embodiments, R₂ is —H, R₃ is —H or methyl, R₄ is —H or —OH, R₅ is —H, R₆ is —H, R, is —H, Rₛ is ethyl, and m is 0, 1, or 2. In some embodiments, R₂ is —H, R₃ is —H or methyl, R₄ is —H, R₅ is —H, R₆ is —H, R₇ is —H, Rₛ is ethyl, and m is 0, 1, or 2. In some embodiments, R₂ is —H, R₃ is —H or methyl, R₄ is —OH, R₅ is —H, R₆ is —H, R₇ is —H, Rₛ is ethyl, and m is 0, 1, or 2.

Various additional embodiments of R₁, R₃, R₄, R₅, R₇, R₈, R¹⁵, R¹⁶, and R¹⁷ are described in WO 2016/130809 (e.g., as R₁, R₂, R₃, R₄, R₅, R₇, Rₐ, R_{c}, and R_{b}, respectively) either individually or in combination (e.g., as in compounds).

In some embodiments, a provided compound is a compound described in WO 2016/130809, wherein its 3-substitutents attached to moiety A are replaced with R¹ and R^{1a}, e.g., in some embodiments, both replaced with —H.

In some embodiments, a provided compound is a compound selected from compounds in Schemes 1-2, Examples 1-2, and Tables 1-4 of WO 2016/130809, wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2016/161003, e.g., a compound of formula (I) or a salt thereof (including stereoisomer, solvate, hydrate or combination thereof) as described in WO 2016/161003, wherein —OR₅ in WO 2016/161003 (in various embodiments, —OR₅ is —OH in WO 2016/161003) is replaced with R¹ or R^{1a} as described herein. In some embodiments, —OR₅ in WO 2016/161003 and the —H that is attached to the same carbon as —OR₅ are replaced with R¹ and R^{1a} as described herein.

In some embodiments, one of R² and R^{2a} is —H and the other is R₈ as described herein (e.g., in some embodiments, R₈ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R is optionally substituted C₁-C₆ aliphatic. In some embodiments, R is optionally substituted C₁-C₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{s0}-L^{s1a}-L^{s1b}-L^{s2}-, wherein each variable is independently as described herein.

In some embodiments, L^{s2} is —O—. In some embodiments, L^{s2} is —OC(O)—. In some embodiments, L^{s2} is —OC(O)N(R′)—. In some embodiments, L^{s2} is —OC(O)N(R_{b})—. In some embodiments, L^{s2} is —OC(O)N(R′)—C(O)N(R′)—. In some embodiments, L^{s2} is —OC(O)N(R′)—C(O)N(R′)S(O)₂—. In some embodiments, L^{s2} is —OC(O)N(R′)—C(O)NHS(O)₂—. In some embodiments, L^{s2} is —OC(O)N(R′)—S(O)₂—. In some embodiments, L^{s2} is —OC(O)N(R′)C(O)—. In some embodiments, L^{s0} is optionally substituted —CH₂—. In some embodiments, L^{s0} is —C(R′)₂—, In some embodiments, L^{s0} is —CHR′—. In some embodiments, L^{s0} is —CH(CH₃)—. In some embodiments, L^{s0} is —(S)—CH(CH₃)—. In some embodiments, L^{s0} is —(R)—CH(CH₃)—. In some embodiments, L^{s1a} is optionally substituted C₁-C₅ alkylene. In some embodiments, L^{s1a} is —(CH₂)m-. In some embodiments, L^{s1b} is optionally substituted —CH₂—. In some embodiments, L^{s1b} is —C(R′)₂—. In some embodiments, L^{s1b} is —CHR′—. In some embodiments, L^{s1b} is —CH(CH₃)—. In some embodiments, L^{s1b} is —(R)—CH(CH₃)—. In some embodiments, Lslb is —(S)—CH(CH₃)—.

In some embodiments, L¹ is —CH(CH₃)—(CH₂)m-CH₂—O—. In some embodiments, L¹ is —CH(CH₃)—(CH₂)m-CH₂—OC(O)—. In some embodiments, L¹ is —CH(CH₃)—(CH₂)m-CH₂—OC(O)N(R′)—. In some embodiments, L¹ is —CH(CH₃)—(CH₂)m-CH₂—OC(O)NH—. In some embodiments, L¹ is —CH(CH₃)—(CH₂)m-CH₂—OC(O)N(R′)—C(O)N(R′)—. In some embodiments, L¹ is —CH(CH₃)—(CH₂)m-CH₂—OC(O)N(R′)—C(O)N(R′)S(O)₂—. In some embodiments, L¹ is —CH(CH₃)—(CH₂)m-CH₂—OC(O)N(R′)—C(O)NHS(O)₂—. In some embodiments, L¹ is —CH(CH₃)—(CH₂)m-CH₂—OC(O)N(R′)—S(O)₂—. In some embodiments, L¹ is —CH(CH₃)—(CH₂)m-CH₂—OC(O)N(R′)—C(O)—. In some embodiments, L¹ is —CH(CH₃)—(CH₂)m-CH(CH₃)—O—. In some embodiments, L¹ is —CH(CH₃)—(CH₂)m-CH(CH₃)—OC(O)—. In some embodiments, L¹ is —CH(CH₃)—(CH₂)m-CH(CH₃)—OC(O)N(R′)—. In some embodiments, L¹ is —CH(CH₃)—(CH₂)m-CH(CH₃)—OC(O)NH—. In some embodiments, L¹ is —CH(CH₃)—(CH₂)m-CH(CH₃)—OC(O)N(R′)—C(O)N(R′)—. In some embodiments, L¹ is —CH(CH₃)—(CH₂)m-CH(CH₃)—OC(O)N(R′)—C(O)N(R′)S(O)₂—. In some embodiments, L¹ is —CH(CH₃)—(CH₂)m-CH(CH₃)—OC(O)N(R′)—C(O)NHS(O)₂—. In some embodiments, L¹ is —CH(CH₃)—(CH₂)m-CH(CH₃)—OC(O)N(R′)—S(O)₂—. In some embodiments, L¹ is —CH(CH₃)—(CH₂)m-CH(CH₃)—OC(O)N(R′)—C(O)—. In some embodiments, —CH(CH₃)— at the end is S; in some embodiments, it is R. In some embodiments, —CH(CH₃)— in the middle is S; in some embodiments, it is R.

In some embodiments, L¹ is -L^{a}-L^{b}-L^{c}-, wherein L^{a} is L^{s0} as described herein, L^{c} is L^{slb} as described herein, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{s1a} is optionally substituted C₁-C₅ alkylene. In some embodiments, L^{s1a} is —(CH₂)m-. In some embodiments, L^{c} is optionally substituted —CH₂—. In some embodiments, L^{c} is —C(R′)₂—. In some embodiments, L^{c} is —CHR′—. In some embodiments, L^{c} is —CH(CH₃)—. In some embodiments, L^{c} is —(R)—CH(CH₃)—. In some embodiments, L^{c} is —(S)—CH(CH₃)—. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, L¹ is —CH(CH₃)(CH₂)mCH₂-, wherein —CH₂— is bonded to R^{L}. In some embodiments, L¹ is (R)—CH(CH₃)(CH₂)mCH₂-, wherein —CH₂— is bonded to R^{L}. In some embodiments, L¹ is (S)—CH(CH₃)(CH₂)mCH₂-, wherein —CH₂— is bonded to R^{L}. In some embodiments, L¹ is (R, R)—CH(CH₃)(CH₂)mCH(CH₃)— which is bonded to R^{L} on its right end. In some embodiments, L¹ is (R, S)—CH(CH₃)(CH₂)mCH(CH₃)— which is bonded to R^{L} on its right end. In some embodiments, L¹ is (S, R)—CH(CH₃)(CH₂)mCH(CH₃)— which is bonded to R^{L} on its right end. In some embodiments, L¹ is (S, S)—CH(CH₃)(CH₂)mCH(CH₃)— which is bonded to R^{L} on its right end.

In some embodiments, R^{L} is —OC(O)N(R^{s})₂. In some embodiments, R^{L} is —OC(O)N(R′)C(O)N(R^{s})₂. In some embodiments, R^{L} is —OC(O)N(R′)C(O)N(R′)S(O)₂R^{s}. In some embodiments, R^{L} is —OC(O)N(R′)C(O)NHS(O)₂R^{s}. In some embodiments, R^{L} is —OC(O)N(R′)S(O)₂R^{s}. In some embodiments, R^{L} is —OC(O)N(R′)C(O)R^{s}. In some embodiments, R^{L} is R^{s}. In some embodiments, R^{L} is R′. In some embodiments, R^{L} is R. In some embodiments, R^{L} is R₁ as described herein.

In some embodiments, a provided compound is a compound of formula VII or a salt thereof:

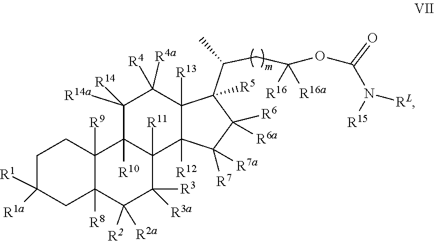

wherein each variable is independently as described herein.

In some embodiments, a provided compound is a compound of a formula selected from formulae VII-1 to VII-12, or a salt thereof, where each variable is independently as described herein.

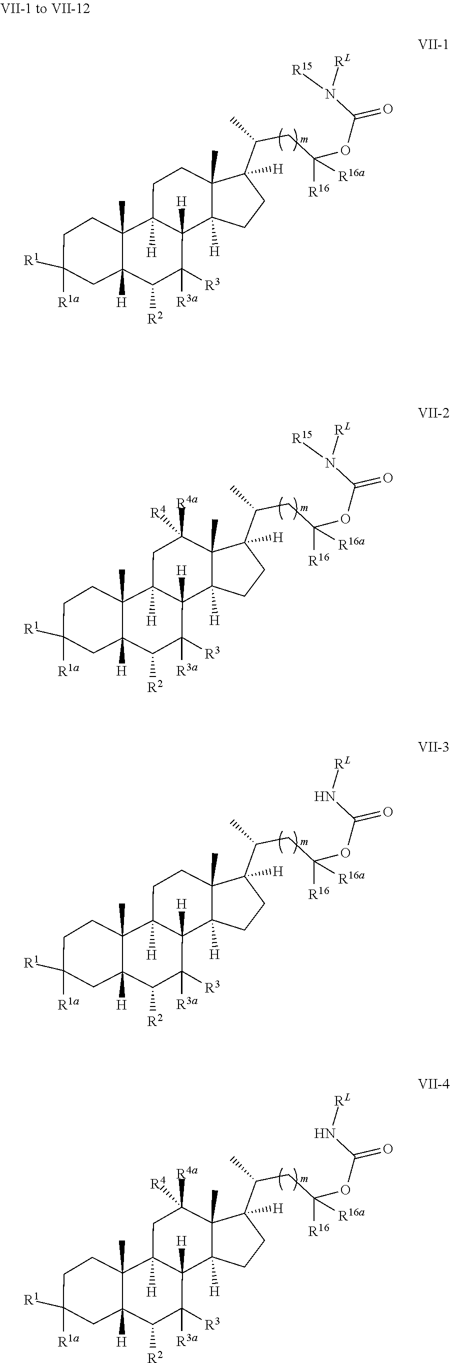

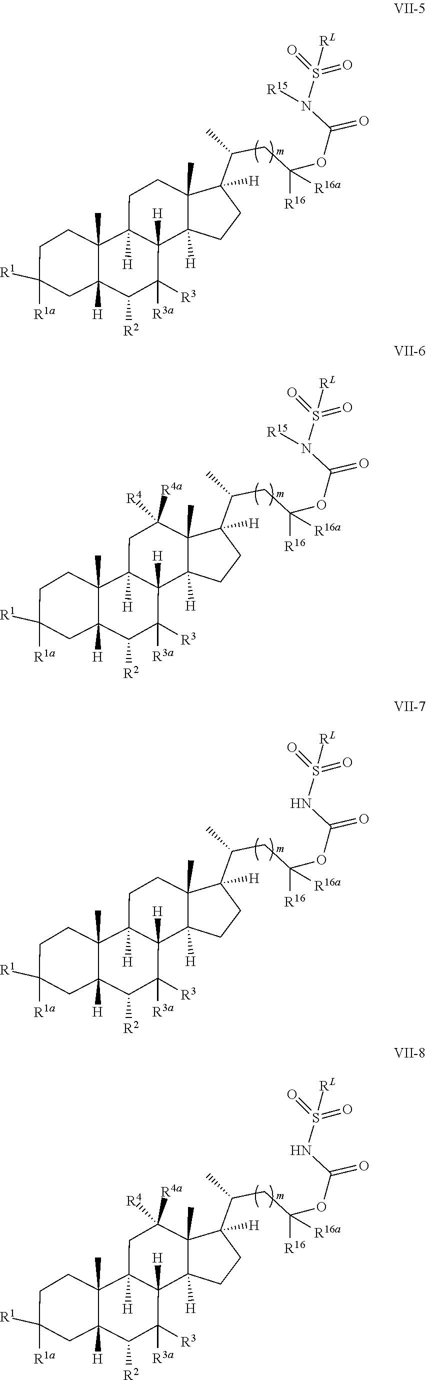

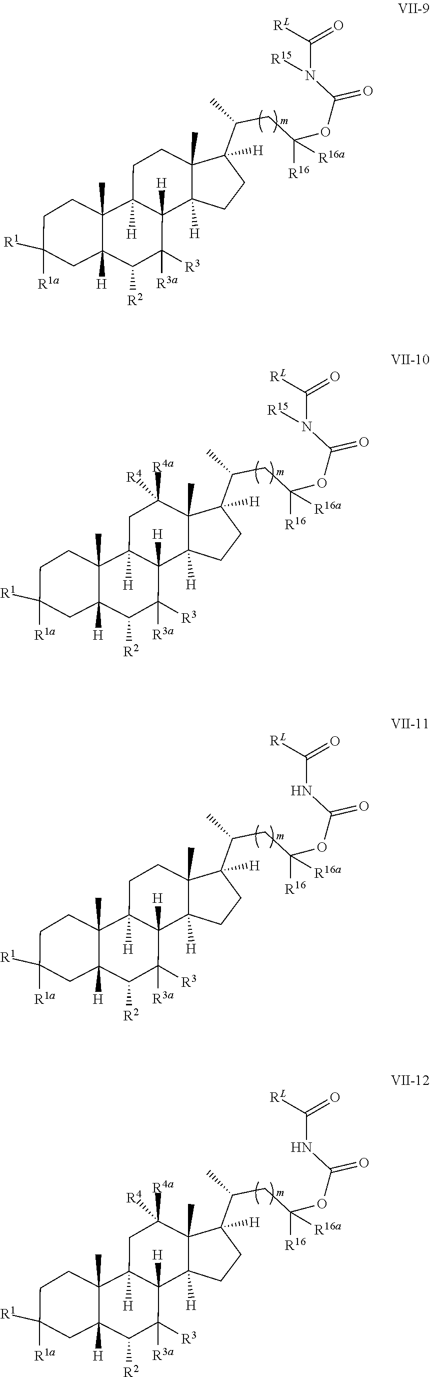

In some embodiments, R^{L} is R_{b} as described herein. In some embodiments, R^{L} is R₁ as described herein. In some embodiments, R¹⁵ is Rₐ as described herein.

In some embodiments, a provided compound is a compound of formula F—I or a salt thereof:

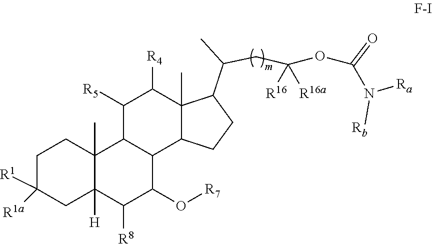

wherein:
   each of R¹ and R^{1a} is independently as described herein;
   R₁ is R₃₀ or R^{s} as described herein;
   R₃₀ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), alkylaryl (e.g., C₁₋₁₂alkylC₆₋₁₄aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), alkylheteroaryl (e.g., C₁₋₁₂alkyl(5-14 membered heteroaryl having 1-10 heteroatoms)), and heteroarylalkyl (e.g., (5-14 membered heteroaryl having 1-10 heteroatoms)C₁₋₁₂alkyl);
   m is 0, 1, 2, or 3;
   R_{b} is R^{L}; and
   each of R₄, R₅, R₇, R₈, R¹⁶, R^{16a}, and Rₐ is independently R^{s} as described herein.

In some embodiments, a provided compound is a compound of formula F—I′ or a salt thereof:

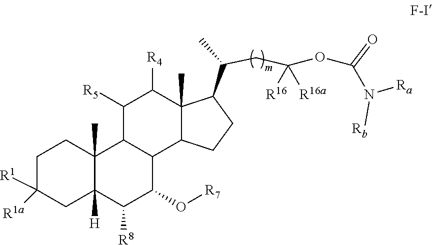

wherein each variable is independently as described herein.

In some embodiments:
each of R¹ and R^{1a} is independently as described herein;
Rₐ is —H or an optionally substituted group selected from C₁₋₆ alkoxy, C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl);
R_{b} is —H, —C(O)NR₉R₁₀, —C(O)NHS(O)₂R₁, —S(O)₂R₁, —C(O)R₁, or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄ arylC₁₋₁₂alkyl); or Rₐ and R_{b} are taken together with the nitrogen atom to which they are attached to form an optionally substituted ring (e.g., an optionally substituted 3-10 membered heterocyclic ring having 1-5 heteroatoms);
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
R¹⁶ is —H, or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R¹⁶ is —H or methyl;
R^{16a} is —H, or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R^{16a} is —H; or R¹⁶ and R^{16a} are taken together with the carbon atom to which they are attached to form an optionally substituted ring (e.g., an optionally substituted C₃₋₁₀ carbocyclic ring);
m is 0, 1, 2, or 3; preferably m is 0, 1, or 2;
R₄ is —H, —OH, —OSO₃H, —OAc or —OPO₃H₂; preferably R₄ is —H or —OH; more preferably —H;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₃ is —H, or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl);
R₇ is —H or a hydroxyl protecting group (e.g., acetyl, trimethylsilyl, or benzyl); preferably R₇ is —H;
R₈ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably R₈ is ethyl; and
R₉ and R₁₀ are each independently —H, or an optionally substituted group selected from C₁₋₈ alkyl, C₂₋₈ alkenyl, C₂₋₈ alkynyl, and C₃₋₈ cycloalkyl, or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H.

In some embodiments, a provided compound is a compound of formula F-IA or a salt thereof:

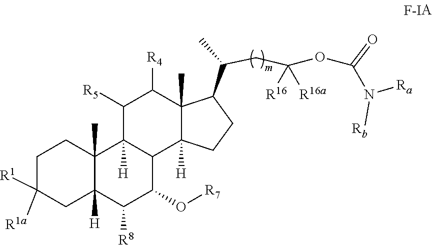

wherein each variable is independently as described herein.

In some embodiments, Rₐ is R₁ as described herein. In some embodiments, R₁ is C₁₋₄ alkyl. In some embodiments, R₁ is C₁₋₄ haloalkyl. In some embodiments, R₁ is C₂₋₄ alkenyl. In some embodiments, R₁ is C₂₋₄ alkynyl. In some embodiments, R₁ is phenyl-C₁₋₄ alkyl. In some embodiments, R₁ is optionally substituted C₃₋₆ cycloalkyl. In some embodiments, R₁ is optionally substituted C₃₋₆ cycloalkyl-C₁₋₄ alkyl. In some embodiments, R₁ is optionally substituted heterocyclyl (e.g., 5- or 6-membered heterocyclyl). In some embodiments, R₁ is optionally substituted 5- or 6-membered heteroaryl. In some embodiments, R₁ is optionally substituted aryl. In some embodiments, R₁ is optionally substituted phenyl. In some embodiments, R₁ is optionally substituted naphthyl. In some embodiments, R₁ is —H. In some embodiments, R₁ is R as described herein. In some embodiments, R₁ is optionally substituted C₁₋₆ alkyl. In some embodiments, R₁ is methyl. In some embodiments, R₁ is ethyl. In some embodiments, R₁ is butyl. In some embodiments, R₁ is t-butyl. In some embodiments, R₁ is propyl. In some embodiments, R₁ is isopropyl. In some embodiments, R₁ is benzyl. In some embodiments, R₁ is vinyl. In some embodiments, R₁ is allyl. In some embodiments, R₁ is —CF₃. In some embodiments, R₁ is cyclopropyl. In some embodiments, R₁ is cyclopentyl. In some embodiments, R₁ is cyclohexyl. In some embodiments, R₁ is 1-methylcyclopropyl. In some embodiments, R₁ is cyclopropylmethyl. In some embodiments, R₁ is optionally substituted phenyl. In some embodiments, R₁ is phenyl. In some embodiments, R₁ is 4-trifluoromethoxyphenyl. In some embodiments, R₁ is 4-t-butylphenyl. In some embodiments, R₁ is 2-methylphenyl. In some embodiments, R₁ is 2-naphthyl. In some embodiments, R₁ is 4-fluorophenyl. In some embodiments, R₁ is 3-fluoro-4-methylphenyl. In some embodiments, R₁ is 4-methylphenyl. In some embodiments, R₁ is 3-pyridinyl. In some embodiments, R₁ is

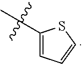 [SMILES: *c1cccs1]

In some embodiments, R₁ is

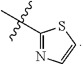 [SMILES: *c1nccs1]

In some embodiments, R_{b} is R₁ as described herein. In some embodiments, R_{b} is R as described herein. In some embodiments, R_{b} is —H. In some embodiments, R_{b} is C₁₋₄ alkyl. In some embodiments, R_{b} is —H. In some embodiments, R_{b} is methyl.

In some embodiments, Rₐ and R_{b} are taken together with the nitrogen atom to which they are attached to form an optionally substituted ring. In some embodiments, a formed ring is an optionally substituted 3-10 (e.g., 3, 4, 5, 6, 7, 8, 9, or 10) membered saturated or partially unsaturated heterocyclyl ring having 1-5 (e.g., 1, 2, 3, 4, or 5) heteroatoms. In some embodiments, a formed ring is an optionally substituted 3-8 membered heterocycloalkyl ring. In some embodiments, a formed ring is an optionally substituted 3-6 membered heterocycloalkyl ring. In some embodiments, a formed ring is an optionally substituted 3-8 membered partially unsaturated heterocyclyl ring. In some embodiments, a formed ring is an optionally substituted 3-6 membered partially unsaturated heterocyclyl ring. In some embodiments, Rₐ and R_{b} are taken together with the nitrogen atom to which they are attached to form optionally substituted

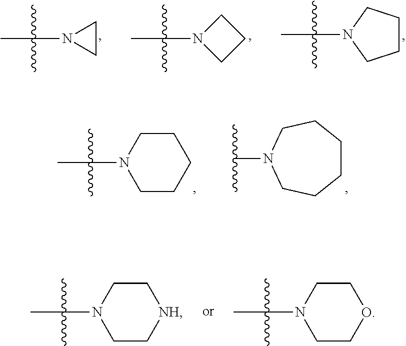

In some embodiments, Rₐ and R_{b} are taken together with the nitrogen atom to which they are attached to form

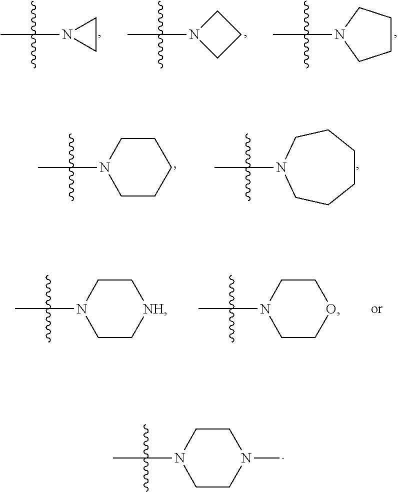

In some embodiments, R_{b} is —C(O)NHS(O)₂R₁. In some embodiments, R_{b} is —S(O)₂R₁. In some embodiments, R_{b} is —C(O)R₁. In some embodiments, R₁ is amino. In some embodiments, R₁ is —NH₂. In some embodiments, R₁ is alkylamino. In some embodiments, R₁ is dialkylamino. In some embodiments, R₁ is halogen. In some embodiments, R₁ is C₁₋₄ alkyl. In some embodiments, R₁ is C₁₋₄ haloalkyl. In some embodiments, R₁ is C₂₋₄ alkenyl. In some embodiments, R₁ is C₂₋₄ alkynyl. In some embodiments, R₁ is phenyl-C₁₋₄ alkyl. In some embodiments, R₁ is optionally substituted C₃₋₆ cycloalkyl. In some embodiments, R₁ is optionally substituted C₃₋₆ cycloalkyl-C₁₋₄ alkyl. In some embodiments, R₁ is optionally substituted heterocyclyl (e.g., 5- or 6-membered heterocyclyl). In some embodiments, R₁ is C₃-C₆-heterocycloalkyl-C₁-C₄-alkyl. In some embodiments, R₁ is optionally substituted 5- or 6-membered heteroaryl. In some embodiments, R₁ is optionally substituted aryl. In some embodiments, R₁ is optionally substituted phenyl. In some embodiments, R₁ is optionally substituted naphthyl. In some embodiments, R₁ is 4-t-butylphenyl. In some embodiments, Rₐ is —H or optionally substituted C₁₋₄ alkyl. In some embodiments, Rₐ is —H. In some embodiments, Rₐ is methyl.

In some embodiments, R₁ is selected from —F, —NH₂, methyl, ethyl, isopropyl, butyl, t-butyl, propyl, benzyl, ally, vinyl, —CF₃, cyclohexyl, cyclopentyl, and the group below:

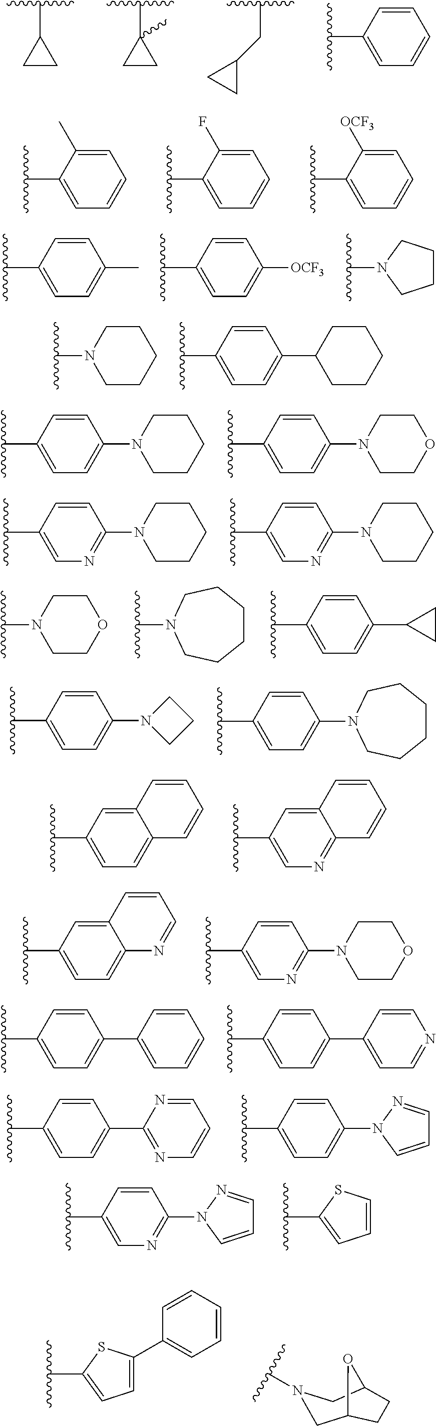

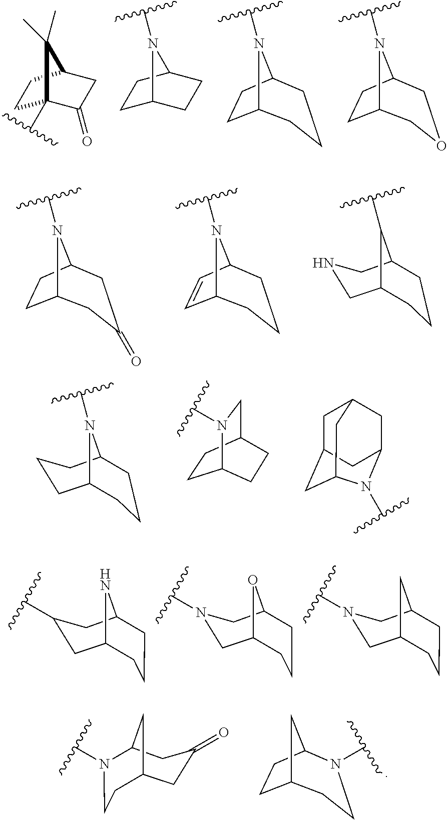

In some embodiments, a provided compound is a compound of formula F-II or a salt thereof:

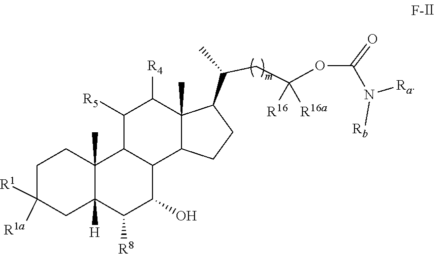

wherein each variable is independently as described herein.

In some embodiments, a provided compound is a compound of formula F—III or a salt thereof:

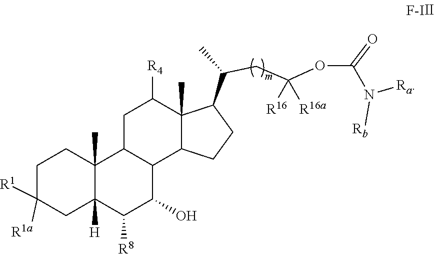

wherein each variable is independently as described herein.

In some embodiments, a provided compound has a structure of a formula selected from F—III-1 to F—III-18, or a salt thereof:

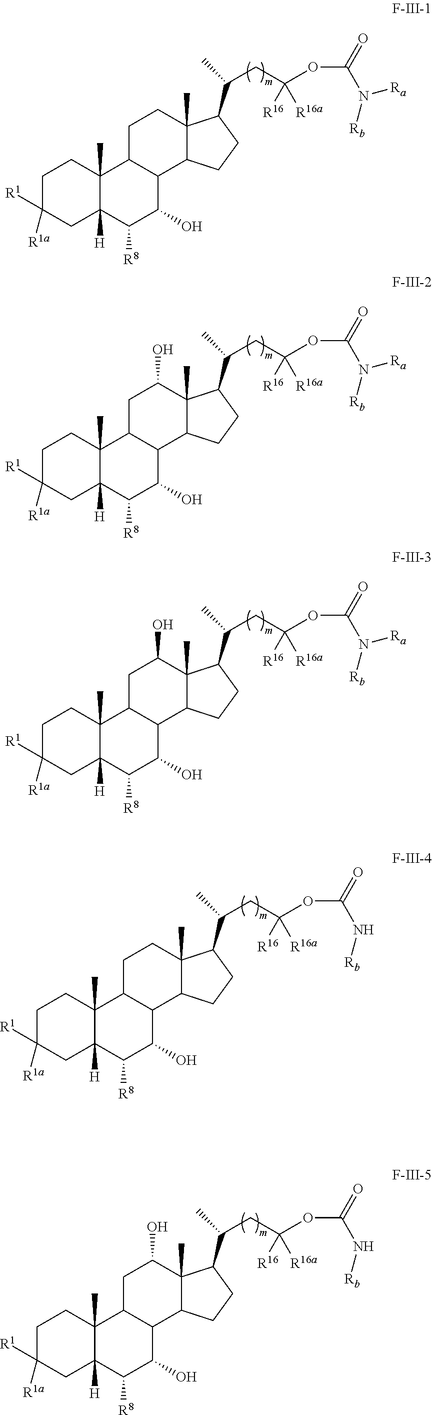

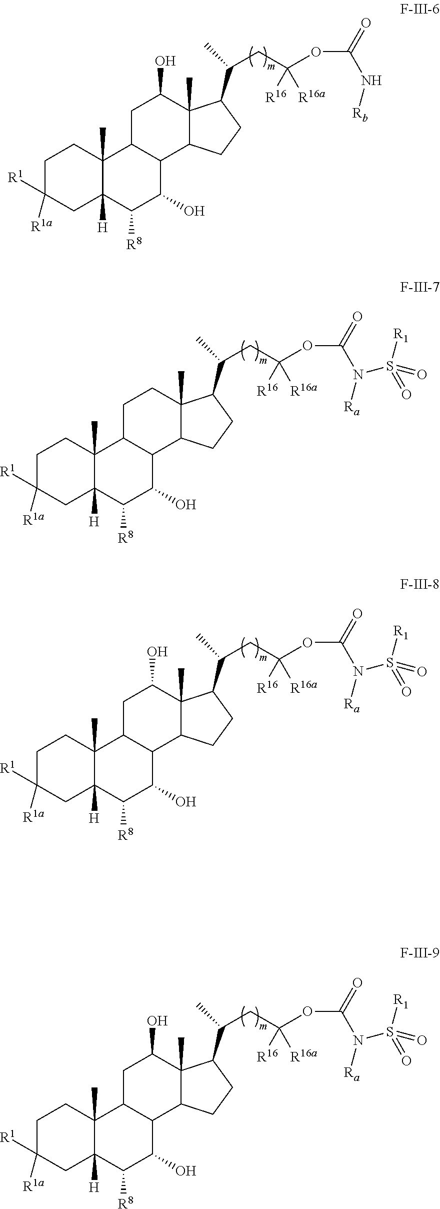

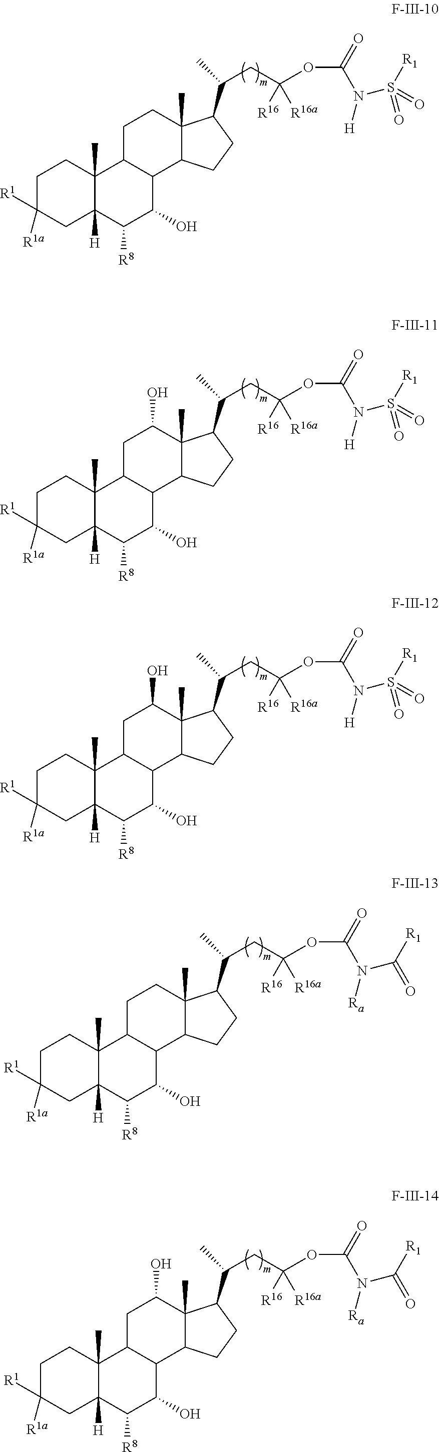

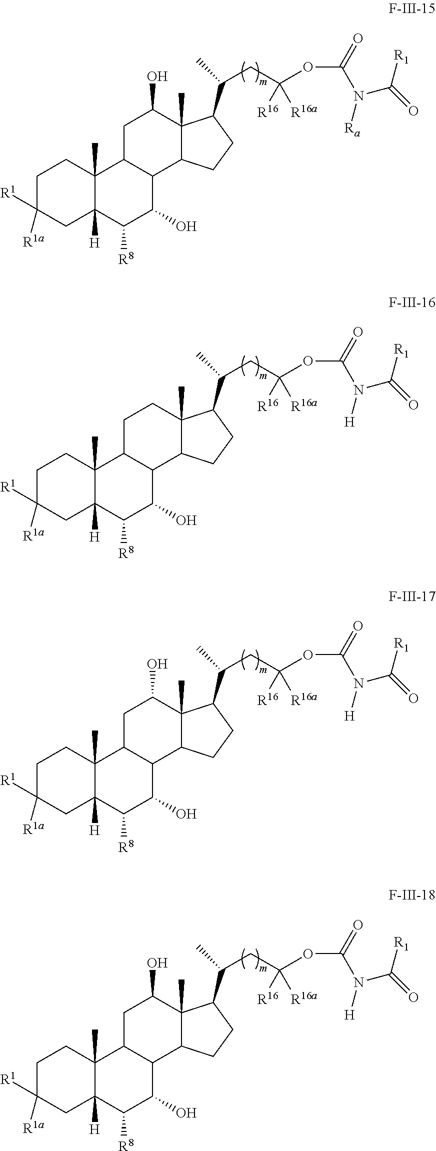

wherein each variable is independently as described herein.

In some embodiments, a provided compound is a compound of formula F-IV or a salt thereof:

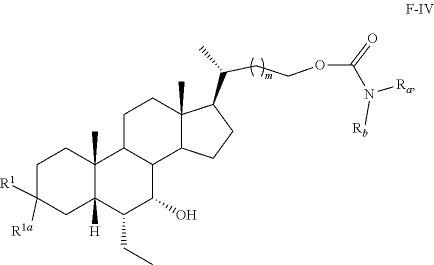

wherein each variable is independently as described herein.

In some embodiments, a provided compound is a compound of formula F-IV-I or a salt thereof:

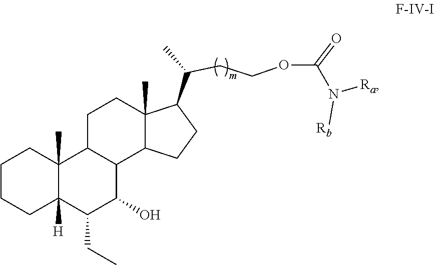

wherein each variable is independently as described herein.

In some embodiments, a provided compound is a compound of formula F-V or a salt thereof:

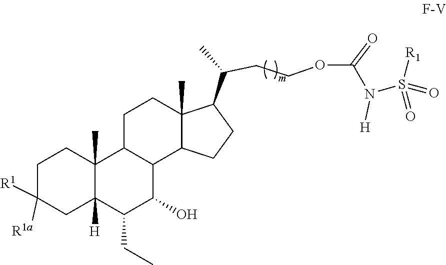

wherein each variable is independently as described herein.

In some embodiments, a provided compound is a compound of formula F-V-I or a salt thereof:

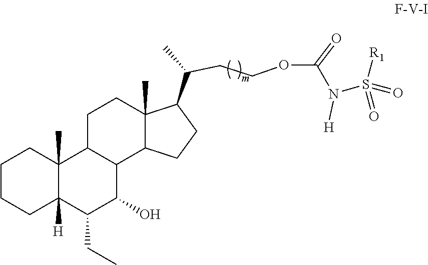

wherein each variable is independently as described herein.

In some embodiments, a provided compound is a compound of formula F-VI or a salt thereof:

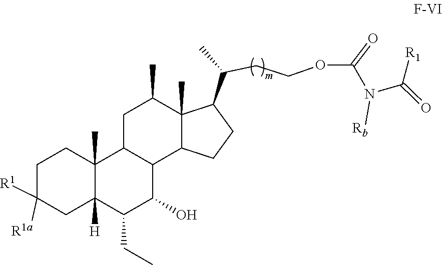

wherein each variable is independently as described herein.

In some embodiments, a provided compound is a compound of formula F-VI-I or a salt thereof:

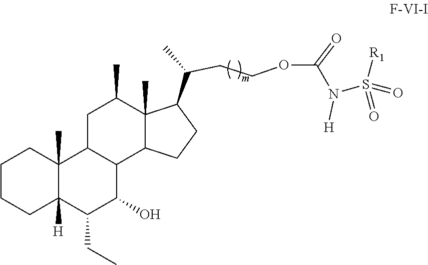

wherein each variable is independently as described herein.

In some embodiments, certain examples of m and R^{L}, and m and R₁, are described in Table 3 below. In some embodiments, m and R₁, or m and R^{L}, are as described in any one of Table 1 to Table 7. In some embodiments, they are as described in Table 1. In some embodiments, they are as described in Table 2. In some embodiments, they are as described in Table 3. In some embodiments, they are as described in Table 4. In some embodiments, they are as described in Table 5. In some embodiments, they are as described in Table 6. In some embodiments, they are as described in Table 7.

In some embodiments, a compound has the structure of formula F-IV or F-IV-I wherein its m, Rₐ and R_{b} is described in Table 3. In some embodiments, a compound has the structure of formula F-IV-I wherein its m and R₁ is described in Table 3 (compounds F-IV-I-1 to F-IV-I-104, wherein F-IV-I-zz has its m and R₇ of entry zz (e.g., compound F-IV-1 has its m and R₇ of entry 1)) or a salt thereof. In some embodiments, a compound has the structure of formula F-V or F-V-I wherein its m and R₇ is described in Table 4. In some embodiments, a compound has the structure of formula F-V-I wherein its m and R₇ is described in Table 4 (compounds F-V-I-1 to F-V-I-78, wherein F-V-I-zz has its m and R₇ of entry zz (e.g., compound F-V-I-1 has its m and R₇ of entry 1)) or a salt thereof. In some embodiments, a compound has the structure of formula F-VI or F-VI-I wherein its m and R₇ is described in Table 5. In some embodiments, a compound has the structure of formula F-VI-I wherein its m and R₇ is described in Table 5 (compounds F-VI-1-1 to F-VI-1-81, wherein F-VI-I-zz has its m and R₇ of entry zz (e.g., compound F-VI-I-1 has its m and R₁ of entry 1)) or a salt thereof. In some embodiments, R^{L} is —OC(O)N(Rₐ)(R_{b}), wherein Rₐ and R_{b} are as described in Table 3 below. In some embodiments, R₇ is —OC(O)N(Rₐ)(R_{b}), wherein Rₐ, and R_{b} are as described in Table 3 below.

TABLE 3
Certain examples of m, Rₐ and R_{b} and R^{L}/R₁(—OC(O)N(Rₐ)(R_{b})).

| Entry | m | Rₐ | R_{b} |
|---|---|---|---|
| 1 | 0 | Methyl | H |
| 2 | 0 | Ethyl | H |
| 3 | 0 | Isopropyl | H |
| 4 | 0 | Butyl | H |
| 5 | 0 | t-Butyl | H |
| 6 | 0 | Propyl | H |
| 7 | 0 | Benzyl | H |
| 8 | 0 | Vinyl | H |
| 9 | 0 | Allyl | H |
| 10 | 0 | CF₃ | H |
| 11 | 0 | 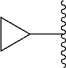 [SMILES: *C1CC1] | H |
| 12 | 0\ | 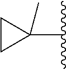 [SMILES: *C1(C)CC1] | H |
| 13 | 0 | 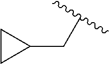 [SMILES: *CC1CC1] | H |
| 14 | 0 | 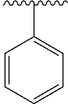 [SMILES: *c1ccccc1] | H |
| 15 | 0 | Methyl | Me |
| 16 | 0 | Ethyl | Me |
| 17 | 0 | Isopropyl | Me |
| 18 | 0 | Butyl | Me |
| 19 | 0 | t-Butyl | Me |
| 20 | 0 | Propyl | Me |
| 21 | 0 | Benzyl | Me |
| 22 | 0 | Vinyl | Me |
| 23 | 0 | Allyl | Me |
| 24 | 0 | CF₃ | Me |
| 25 | 0 | 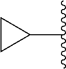 [SMILES: *C1CC1] | Me |
| 26 | 0 | 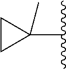 [SMILES: *C1(C)CC1] | Me |
| 27 | 0 | 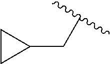 [SMILES: *CC1CC1] | Me |
| 28 | 0 | 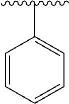 [SMILES: *c1ccccc1] | Me |
| 29 | 0 | 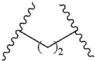 | |
| 30 | 0 | 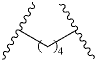 | |
| 31 | 0 | 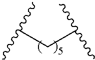 | |
| 32 | 0 | 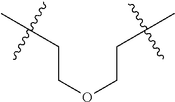 [SMILES: *CCOCC*] | |
| 33 | 0 | 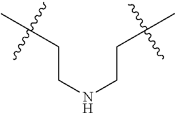 [SMILES: *CCNCC*] | |
| 34 | 0 | 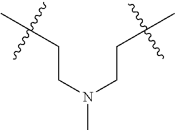 [SMILES: *CCN(C)CC*] | |
| 37 | 1 | Methyl | H |
| 38 | 1 | Ethyl | H |
| 39 | 1 | Isopropyl | H |
| 40 | 1 | Butyl | H |
| 41 | 1 | t-Butyl | H |
| 42 | 1 | Propyl | H |
| 43 | 1 | Benzyl | H |
| 44 | 1 | Vinyl | H |
| 45 | 1 | Allyl | H |
| 46 | 1 | CF₃ | H |
| 47 | 1 | 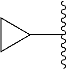 [SMILES: *C1CC1] | H |
| 48 | 1 | 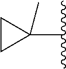 [SMILES: *C1(C)CC1] | H |
| 49 | 1 | 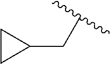 [SMILES: *CC1CC1] | H |
| 50 | 1 | 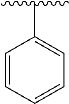 [SMILES: *c1ccccc1] | H |
| 51 | 1 | Methyl | Me |
| 52 | 1 | Ethyl | Me |
| 53 | 1 | Isopropyl | Me |
| 54 | 1 | Butyl | Me |
| 55 | 1 | t-Butyl | Me |
| 56 | 1 | Propyl | Me |
| 57 | 1 | Benzyl | Me |
| 58 | 1 | Vinyl | Me |
| 59 | 1 | Allyl | Me |
| 60 | 1 | CF₃ | Me |
| 61 | 1 | 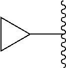 [SMILES: *C1CC1] | Me |
| 62 | 1 | 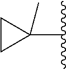 [SMILES: *C1(C)CC1] | Me |
| 63 | 1 | 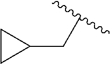 [SMILES: *CC1CC1] | Me |
| 64 | 1 | 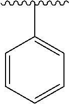 [SMILES: *c1ccccc1] | Me |
| 65 | 1 | 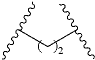 | |
| 66 | 1 | 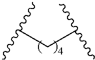 | |
| 67 | 1 | 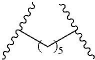 | |
| 68 | 1 | 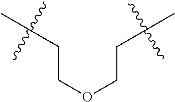 [SMILES: *CCOCC*] | |
| 69 | 1 | 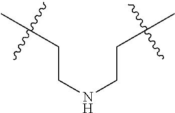 [SMILES: *CCNCC*] | |
| 70 | 1 | 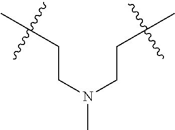 [SMILES: *CCN(C)CC*] | |
| 71 | 2 | Methyl | H |
| 72 | 2 | Ethyl | H |
| 73 | 2 | Isopropyl | H |
| 74 | 2 | Butyl | H |
| 75 | 2 | t-Butyl | H |
| 76 | 2 | Propyl | H |
| 77 | 2 | Benzyl | H |
| 78 | 2 | Vinyl | H |
| 79 | 2 | Allyl | H |
| 80 | 2 | CF₃ | H |
| 81 | 2 | 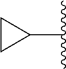 [SMILES: *C1CC1] | H |
| 82 | 2 | 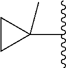 [SMILES: *C1(C)CC1] | H |
| 83 | 2 | 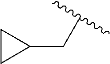 [SMILES: *CC1CC1] | H |
| 84 | 2 | 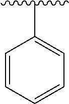 [SMILES: *c1ccccc1] | H |
| 85 | 2 | Methyl | Me |
| 86 | 2 | Ethyl | Me |
| 87 | 2 | Isopropyl | Me |
| 88 | 2 | Butyl | Me |
| 89 | 2 | t-Butyl | Me |
| 90 | 2 | Propyl | Me |
| 91 | 2 | Benzyl | Me |
| 92 | 2 | Vinyl | Me |
| 93 | 2 | Allyl | Me |
| 94 | 2 | CF₃ | Me |
| 95 | 2 | 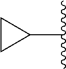 [SMILES: *C1CC1] | Me |
| 96 | 2 | 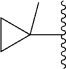 [SMILES: *C1(C)CC1] | Me |
| 97 | 2 | 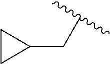 [SMILES: *CC1CC1] | Me |
| 98 | 2 | 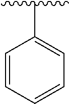 [SMILES: *c1ccccc1] | Me |

| | | |
|---|---|---|
| 99 | 2 | 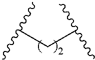 |
| 100 | 2 | 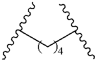 |
| 101 | 2 | 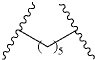 |
| 102 | 2 | 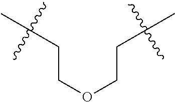 [SMILES: *CCOCC*] |
| 103 | 2 | 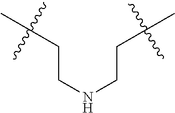 [SMILES: *CCNCC*] |
| 104 | 2 | 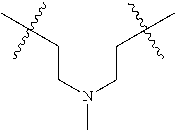 [SMILES: *CCN(C)CC*] |

TABLE 4
Certain examples of m and R^{L}/R₁.

| Entry | m | R^{L}/R₁ |
|---|---|---|
| 1 | 0 | Methyl |
| 2 | 0 | Ethyl |
| 3 | 0 | Isopropyl |
| 4 | 0 | Butyl |
| 5 | 0 | t-Butyl |
| 6 | 0 | Propyl |
| 7 | 0 | Benzyl |
| 8 | 0 | Vinyl |
| 9 | 0 | Allyl |
| 10 | 0 | 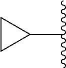 [SMILES: *C1CC1] |
| 11 | 0 | 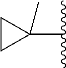 [SMILES: *C1(C)CC1] |
| 12 | 0 | 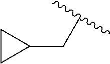 [SMILES: *CC1CC1] |
| 13 | 0 | 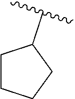 [SMILES: *C1CCCC1] |
| 14 | 0 | 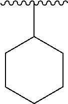 [SMILES: *C1CCCCC1] |
| 15 | 0 | 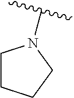 [SMILES: *N1CCCC1] |
| 16 | 0 | 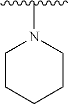 [SMILES: *N1CCCCC1] |
| 17 | 0 | 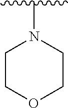 [SMILES: *N1CCOCC1] |
| 18 | 0 | NH₂ |
| 19 | 0 | 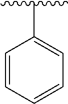 [SMILES: *c1ccccc1] |
| 20 | 0 | 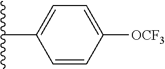 [SMILES: *c1ccc(OC(F)(F)F)cc1] |
| 21 | 0 | 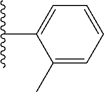 [SMILES: *c1ccccc1C] |
| 22 | 0 | 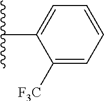 [SMILES: *c1ccccc1C(F)(F)F] |
| 23 | 0 | 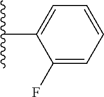 [SMILES: *c1ccccc1F] |
| 24 | 0 | 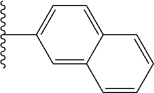 [SMILES: *c1ccc2ccccc2c1] |
| 25 | 0 | 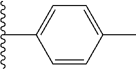 [SMILES: *c1ccc(C)cc1] |
| 26 | 0 | F |
| 27 | 1 | Methyl |
| 28 | 1 | Ethyl |
| 29 | 1 | Isopropyl |
| 30 | 1 | Butyl |
| 31 | 1 | t-Butyl |
| 32 | 1 | Propyl |
| 33 | 1 | Benzyl |
| 34 | 1 | Vinyl |
| 35 | 1 | Allyl |
| 36 | 1 | 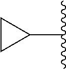 [SMILES: *C1CC1] |
| 37 | 1 | 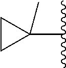 [SMILES: *C1(C)CC1] |
| 38 | 1 | 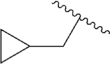 [SMILES: *CC1CC1] |
| 39 | 1 | 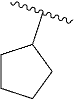 [SMILES: *C1CCCC1] |
| 40 | 1 | 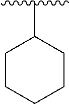 [SMILES: *C1CCCCC1] |
| 41 | 1 | 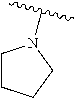 [SMILES: *N1CCCC1] |
| 42 | 1 | 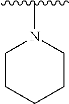 [SMILES: *N1CCCCC1] |
| 43 | 1 | 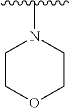 [SMILES: *N1CCOCC1] |
| 44 | 1 | NH₂ |
| 45 | 1 | 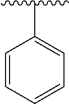 [SMILES: *c1ccccc1] |
| 46 | 1 | 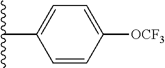 [SMILES: *c1ccc(OC(F)(F)F)cc1] |
| 47 | 1 | 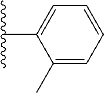 [SMILES: *c1ccccc1C] |
| 48 | 1 | 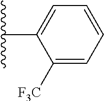 [SMILES: *c1ccccc1C(F)(F)F] |
| 49 | 1 | 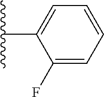 [SMILES: *c1ccccc1F] |
| 50 | 1 | 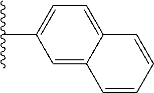 [SMILES: *c1ccc2ccccc2c1] |
| 51 | 1 | 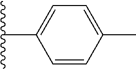 [SMILES: *c1ccc(C)cc1] |
| 52 | 1 | F |
| 53 | 2 | Methyl |
| 54 | 2 | Ethyl |
| 55 | 2 | Isopropyl |
| 56 | 2 | Butyl |
| 57 | 2 | t-Butyl |
| 58 | 2 | Propyl |
| 59 | 2 | Benzyl |
| 60 | 2 | Vinyl |
| 61 | 2 | Allyl |
| 62 | 2 | 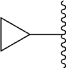 [SMILES: *C1CC1] |
| 63 | 2 | 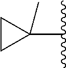 [SMILES: *C1(C)CC1] |
| 64 | 2 | 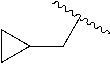 [SMILES: *CC1CC1] |
| 65 | 2 | 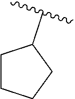 [SMILES: *C1CCCC1] |
| 66 | 2 | 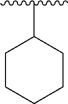 [SMILES: *C1CCCCC1] |
| 67 | 2 | 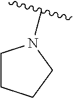 [SMILES: *N1CCCC1] |
| 68 | 2 | 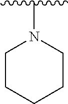 [SMILES: *N1CCCCC1] |
| 69 | 2 | 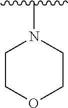 [SMILES: *N1CCOCC1] |
| 70 | 2 | NH₂ |
| 71 | 2 | 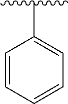 [SMILES: *c1ccccc1] |
| 72 | 2 | 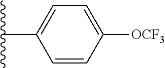 [SMILES: *c1ccc(OC(F)(F)F)cc1] |
| 73 | 2 | 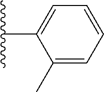 [SMILES: *c1ccccc1C] |
| 74 | 2 | 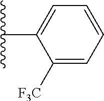 [SMILES: *c1ccccc1C(F)(F)F] |
| 75 | 2 | 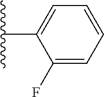 [SMILES: *c1ccccc1F] |
| 76 | 2 | 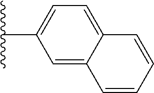 [SMILES: *c1ccc2ccccc2c1] |
| 77 | 2 | 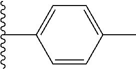 [SMILES: *c1ccc(C)cc1] |
| 78 | 2 | F |

TABLE 5
Certain examples of m and R^{L}/R₁.

| Entry | m | R^{L}/R₁ |
|---|---|---|
| 1 | 0 | Methyl |
| 2 | 0 | Ethyl |
| 3 | 0 | Isopropyl |
| 4 | 0 | Butyl |
| 5 | 0 | t-Butyl |
| 6 | 0 | Propyl |
| 7 | 0 | Benzyl |
| 8 | 0 | Vinyl |
| 9 | 0 | Allyl |
| 10 | 0 | CF₃ |
| 11 | 0 | 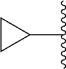 [SMILES: *C1CC1] |
| 12 | 0 | 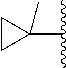 [SMILES: *C1(C)CC1] |
| 13 | 0 | 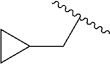 [SMILES: *CC1CC1] |
| 14 | 0 | 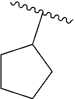 [SMILES: *C1CCCC1] |
| 15 | 0 | 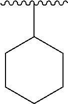 [SMILES: *C1CCCCC1] |
| 16 | 0 | 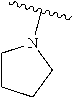 [SMILES: *N1CCCC1] |
| 17 | 0 | 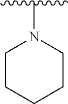 [SMILES: *N1CCCCC1] |
| 18 | 0 | 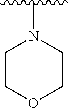 [SMILES: *N1CCOCC1] |
| 19 | 0 | NH₂ |
| 20 | 0 | 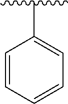 [SMILES: *c1ccccc1] |
| 21 | 0 | 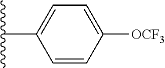 [SMILES: *c1ccc(OC(F)(F)F)cc1] |
| 22 | 0 | 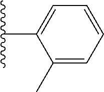 [SMILES: *c1ccccc1C] |
| 23 | 0 | 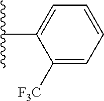 [SMILES: *c1ccccc1C(F)(F)F] |
| 24 | 0 | 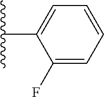 [SMILES: *c1ccccc1F] |
| 25 | 0 | 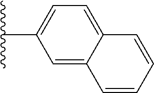 [SMILES: *c1ccc2ccccc2c1] |
| 26 | 0 | 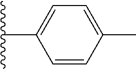 [SMILES: *c1ccc(C)cc1] |
| 27 | 0 | F |
| 28 | 2 | Methyl |
| 29 | 2 | Ethyl |
| 30 | 2 | Isopropyl |
| 31 | 2 | Butyl |
| 32 | 2 | t-Butyl |
| 33 | 2 | Propyl |
| 34 | 2 | Benzyl |
| 35 | 2 | Vinyl |
| 36 | 2 | Allyl |
| 37 | | CF₃ |
| 38 | 2 | 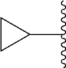 [SMILES: *C1CC1] |
| 39 | 2 | 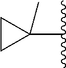 [SMILES: *C1(C)CC1] |
| 40 | 2 | 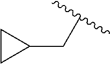 [SMILES: *CC1CC1] |
| 41 | 2 | 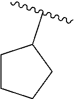 [SMILES: *C1CCCC1] |
| 42 | 2 | 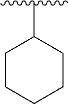 [SMILES: *C1CCCCC1] |
| 43 | 2 | 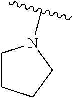 [SMILES: *N1CCCC1] |
| 44 | 2 | 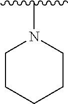 [SMILES: *N1CCCCC1] |
| 45 | 2 | 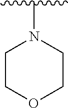 [SMILES: *N1CCOCC1] |
| 46 | 2 | NH₂ |
| 47 | 2 | 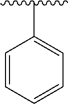 [SMILES: *c1ccccc1] |
| 48 | 2 | 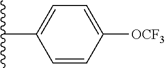 [SMILES: *c1ccc(OC(F)(F)F)cc1] |
| 49 | 2 | 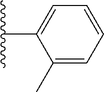 [SMILES: *c1ccccc1C] |
| 50 | 2 | 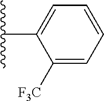 [SMILES: *c1ccccc1C(F)(F)F] |
| 51 | 2 | 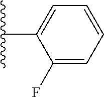 [SMILES: *c1ccccc1F] |
| 52 | 2 | 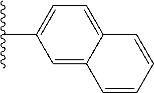 [SMILES: *c1ccc2ccccc2c1] |
| 53 | 2 | 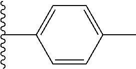 [SMILES: *c1ccc(C)cc1] |
| 54 | 2 | F |
| 55 | 3 | Methyl |
| 56 | 3 | Ethyl |
| 57 | 3 | Isopropyl |
| 58 | 3 | Butyl |
| 59 | 3 | t-Butyl |
| 60 | 3 | Propyl |
| 61 | 3 | Benzyl |
| 62 | 3 | Vinyl |
| 63 | 3 | Allyl |
| 64 | 3 | CF₃ |
| 65 | 3 | 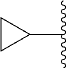 [SMILES: *C1CC1] |
| 66 | 3 | 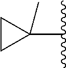 [SMILES: *C1(C)CC1] |
| 67 | 3 | 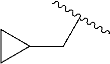 [SMILES: *CC1CC1] |
| 68 | 3 | 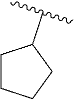 [SMILES: *C1CCCC1] |
| 69 | 3 | 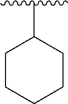 [SMILES: *C1CCCCC1] |
| 70 | 3 | 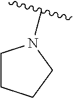 [SMILES: *N1CCCC1] |
| 71 | 3 | 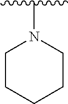 [SMILES: *N1CCCCC1] |
| 72 | 3 | 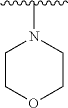 [SMILES: *N1CCOCC1] |
| 73 | 3 | NH₂ |
| 74 | 3 | 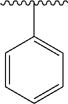 [SMILES: *c1ccccc1] |
| 75 | 3 | 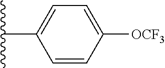 [SMILES: *c1ccc(OC(F)(F)F)cc1] |
| 76 | 3 | 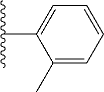 [SMILES: *c1ccccc1C] |
| 77 | 3 | 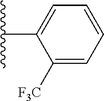 [SMILES: *c1ccccc1C(F)(F)F] |
| 78 | 3 | 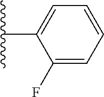 [SMILES: *c1ccccc1F] |
| 79 | 3 | 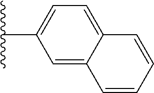 [SMILES: *c1ccc2ccccc2c1] |
| 80 | 3 | 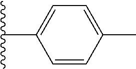 [SMILES: *c1ccc(C)cc1] |
| 81 | 3 | F |

In some embodiments, R¹⁶ is —H. In some embodiments, R^{16a} is —H. In some embodiments, R₃ is —H. In some embodiments, R₃ is methyl. In some embodiments, R₄ is —H. In some embodiments, R₄ is —OH. In some embodiments, Rₛ is —H. In some embodiments, R₆ is —H. In some embodiments, R₇ is —H. In some embodiments, R₈ is ethyl. In some embodiments, m is 0. In some embodiments, m is 1. In some embodiments, m is 2.

In some embodiments, R¹⁶ is —H, R^{16a} is —H or methyl, R₄ is —H or —OH, R₅ is —H, R₆ is —H, R₇ is —H, R₈ is ethyl, and m is 0, 1, or 2. In some embodiments, R¹⁶ is —H, R^{16a} is —H or methyl, R₄ is —H, Rₛ is —H, R₆ is —H, R₇ is —H, R₈ is ethyl, and m is 0, 1, or 2. In some embodiments, R¹⁶ is —H, R^{16a} is —H or methyl, R₄ is —OH, R₅ is —H, R₆ is —H, R₇ is —H, R₈ is ethyl, and m is 0, 1, or 2.

Various additional embodiments of R₁, R₄, R₅, R₇, R₈, R¹⁶, R^{16a}, Rₐ and R_{b} are described in WO 2016/161003 (e.g., as R₁, R₃, R₄, R₆, R₇, R₂, R_{c}, Rₐ and R_{b}, respectively) either individually or in combination (e.g., as in compounds).

In some embodiments, a provided compound is a compound described in WO 2016/161003, wherein its 3-substitutents attached to moiety A are replaced with R¹ and R^{1a}, e.g., in some embodiments, both replaced with —H.

In some embodiments, a provided compound is a compound selected from compounds 1-261 of WO 2016/161003, wherein its 3-OH and 3-H attached to moiety A are replaced with R¹ and R^{1a}, e.g., in some embodiments, both replaced with —H.

In some embodiments, a provided compound is a compound selected from compounds in Schemes 1-5, Examples 1-218, and Tables 1-7 of WO 2016/161003, wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 9 of WO 2016/161003, wherein the compound has an EC50 labeled as “A” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 9 of WO 2016/161003, wherein the compound has an EC50 labeled as “B” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 9 of WO 2016/161003, wherein the compound has an EC50 labeled as “C” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 9 of WO 2016/161003, wherein the compound has an EC50 labeled as “D” and wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2017/147137, e.g., a compound of formula (I) or a salt thereof (including stereoisomer, solvate, hydrate or combination thereof) as described in WO 2017/147137, wherein R₄ in WO 2017/147137 (in various embodiments, R₄ is —OH in WO 2017/147137) is replaced with R¹ or R^{1a} as described herein. In some embodiments, R₄ in WO 2017/147137 and the —H that is attached to the same carbon as R⁴ are replaced with R¹ and R^{1a} as described herein.

In some embodiments, one of R² and R^{2a} is —H and the other is R₈ as described herein (e.g., in some embodiments, R₈ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R is optionally substituted C₁₋₆ aliphatic. In some embodiments, R is optionally substituted C₁₋₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{a}-L^{b}-L^{c}-, wherein L^{a} is L^{s0} as described herein, L^{c} is -Cy- as described herein, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L¹ is -L^{a}-L^{b}-L^{c}-, wherein L^{a} is L^{s0} as described herein, L^{c} is a covalent bond, —C(O)N(R′)—, or —N(R′)—, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(S)N(R′)—, —C(O)N(R′)—, —C(NR′)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{s1a} is optionally substituted C₁₋₅ alkylene. In some embodiments, L^{s1a} is —(CH₂)m-. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, L^{c} is -Cy- wherein -Cy- is an optionally substituted bivalent aromatic ring. In some embodiments, -Cy- is optionally substituted phenylene. In some embodiments, -Cy-is optionally substituted 5-14 membered heteroarylene having 1-10 (e.g., 1, 2, 3, 4, 5, 6, 7, 8, 9, or 10) heteroatoms. In some embodiments, -Cy- is optionally substituted 5-membered heteroarylene having 1-4 (e.g., 1, 2, 3 or 4) heteroatoms. In some embodiments, -Cy- is optionally substituted 6-membered heteroarylene having 1-4 (e.g., 1, 2, 3 or 4) heteroatoms. In some embodiments, -Cy- is optionally substituted 9-membered bicyclic heteroarylene having 1-6 (e.g., 1, 2, 3, 4, 5 or 6) heteroatoms. In some embodiments, -Cy- is optionally substituted 10-membered bicyclic heteroarylene having 1-6 (e.g., 1, 2, 3, 4, 5 or 6) heteroatoms. In some embodiments, -Cy- is an optionally substituted bivalent phenyl, pyridyl, pyrimidyl, pyrazinyl, pyridazinyl, triazinyl, tetrazinyl, tetrazolyl, indolyl, indazolyl, thienyl, benzothienyl, furanyl, benzofuranyl, isoxazolyl, benzoxazolyl, thiazolyl, benzothiazolyl, benzotriazolyl, imidazolyl, or benzoimidazolyl ring. In some embodiments, L^{c} is a covalent bond. In some embodiments, L^{c} is —N(R′)—. In some embodiments, L^{c} is —NH—. In some embodiments, L^{c} is —C(O)N(R′)—. In some embodiments, L^{c} is —C(O)NH—.

In some embodiments, R^{L} is R^{s}. In some embodiments, R^{L} is R′. In some embodiments, R^{L} is R. In some embodiments, R^{L} is —H. In some embodiments, R^{L} is —C(O)R^{s}. In some embodiments, R^{L} is —C(O)N(R^{s})₂. In some embodiments, R^{L} is —C(O)N(R′)S(O)₂R^{s}. In some embodiments, R^{L} is —P(O)(R^{s})₂. In some embodiments, R^{L} is —CN.

In some embodiments, R^{L} is —H. In some embodiments, R^{L} is optionally substituted heteroaryl. In some embodiments, R^{L} is optionally substituted 5-membered heteroaryl having 1-4 heteroatoms. In some embodiments, R^{L} is optionally substituted tetrazolyl. In some embodiments, R^{L} is tetrazolyl.

In some embodiments, a provided compound is a compound of the formula below or a salt thereof:

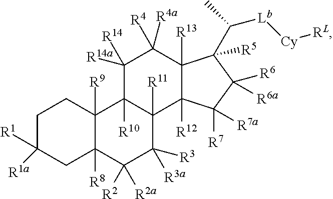

wherein:
   L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—; and
   each variable is independently as described herein.

In some embodiments, -Cy- is —Ar—, wherein —Ar— is an optionally substituted bivalent aryl (e.g., 6-14 membered) or heteroaryl (e.g., 5-14 membered having 1-10 heteroatoms) ring.

In some embodiments, a provided compound is a compound of the formula below or a salt thereof:

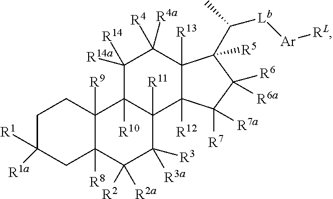

wherein each variable is independently as described herein.

In some embodiments, a provided compound is a compound of formula XIV or a salt thereof:

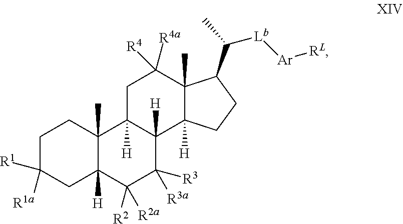

wherein each variable is independently as described herein.

In some embodiments, a provided compound is a compound of formula XIV-1 or a salt thereof:

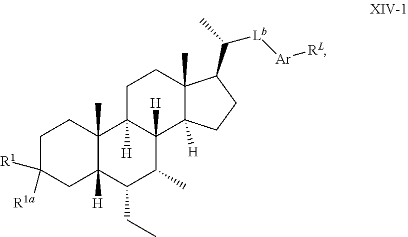

wherein each variable is independently as described herein.

In some embodiments, R^{L} is —H, —C(O)R₁₁, —C(O)NR₁₂R₁₃, —C(O)NHS(O)₂R₁₁, —P(O)(R₁₁)₂, —CN, or tetrazolyl;
Ar is a bivalent optionally substituted aryl (e.g., C₆₋₁₄ aryl) or heteroaryl (e.g., 5-14 membered heteroaryl having 1-10 heteroatoms) ring; preferably, Ar is a bivalent phenyl, pyridyl, pyrimidyl, pyrazinyl, pyridazinyl, triazinyl, tetrazinyl, tetrazolyl, indolyl, indazolyl, thienyl, benzothienyl, furanyl, benzofuranyl, isoxazolyl, benzoxazolyl, thiazolyl, benzothiazolyl, benzotriazolyl, imidazolyl, or benzoimidazolyl ring, each of which is optionally substituted;
L^{b} is an optionally substituted group selected from C₂₋₈ alkylene, C₂₋₈ alkenylene, C₂₋₈ alkynylene, C₃₋₈ cycloalkylene, C₁₋₇ alkoxylene, C₁₋₇ aminoalkylene, C₃_₇ heterocycloalkylene, C₁₋₈ alkylene-C₃₋₈ cycloalkylene, C₁₋₈ alkylene-C₃₋₇ heterocycloalkylene (e.g., having 1-5 hetereoatoms), bivalent alkylaryl (e.g., C₁₋₈ alkylene-6-14 membered arylene), alkylheteroaryl (e.g., C₁₋₈ alkylene-5-14 membered heteroarylene having 1-5 heteroatoms);
R¹ is —H (in some embodiments, is deuterium), halogen or optionally substituted methyl (e.g., methyl or —CF₃);
R^{1a} is —H (in some embodiments, is deuterium), or halogen;
R₁₁ is —H, halogen, —OH, or an optionally substituted group selected from C₁₋₈ alkoxy, C₁₋₈ alkyl, C₂₋₈ alkenyl, C₂₋₈ alkynyl, C₃₋₈ cycloalkyl, C₃₋₈ cycloalkenyl, C₃₋₈ heterocycloalkyl, aryl (e.g., C₆₋₁₄ aryl), arylalkyl (e.g., C₆₋₁₄arylC₁₋₁₂alkyl), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl);
each R₁₂ and R₁₃ is independently —H, or an optionally substituted group selected from C₁₋₈ alkyl, C₂₋₈ alkenyl, C₂₋₈ alkynyl, C₃₋₈ cycloalkyl, 3- to 8-membered heterocyclyl (e.g., having 1-5 heteroatoms), aryl (e.g., C₆₋₁₄ aryl), arylalkyl (e.g., C₆₋₁₄arylC₁₋₁₂alkyl), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl); preferably R₁₂ is an optionally substituted group selected from C₁₋₈ alkyl and aryl; and/or preferably R₁₃ is —H; alternatively R₁₂ an R¹³ are taken together with the nitrogen atom to which they are attached to form an optionally substituted heterocyclic ring (e.g., an optionally substituted 3-15 membered, monocyclic, bicyclic or polycyclic heterocyclyl ring having 1-5 heteroatoms); and
each other variable is independently as described herein.

In some embodiments, R^{L} is —H. In some embodiments, R^{L} is optionally substituted tetrazolyl. In some embodiments, R^{L} is tetrazolyl.

In some embodiments, a provided compound is a compound of formula XIV-II or a salt thereof:

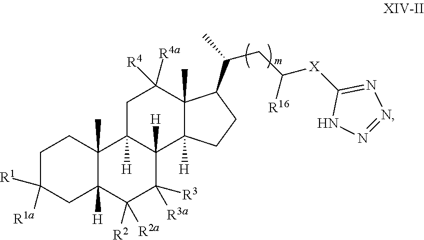

wherein:
   X is a covalent bond, —C(O)NH— or —NH—,
   R¹⁶ is —H or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R¹⁶ is —H or methyl;
   m is 0, 1, 2, or 3; preferably m is 0, 1, or 2;
   each other variable is independently as described herein.

In some embodiments, a provided compound is a compound of a formula selected below, or a salt thereof:

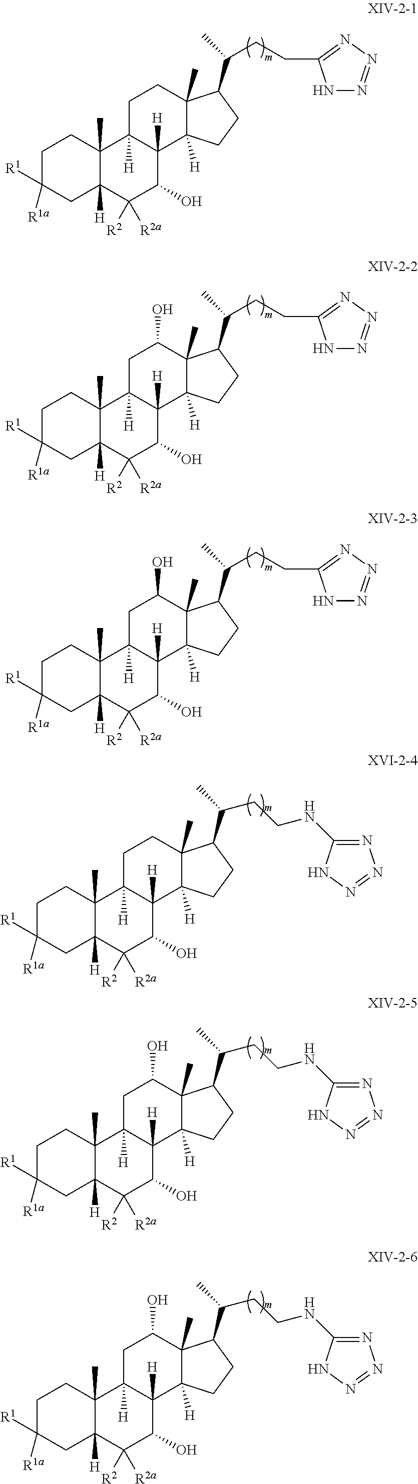

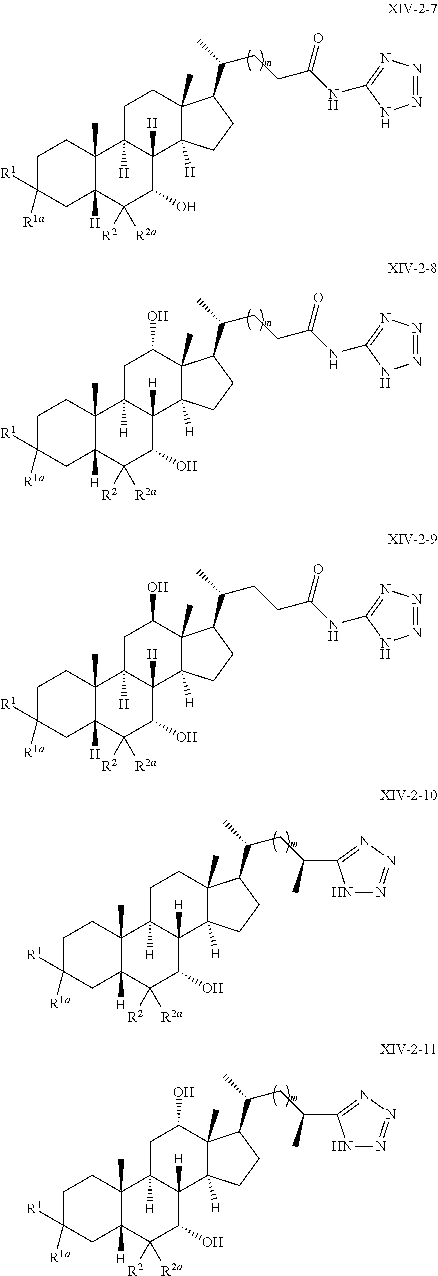

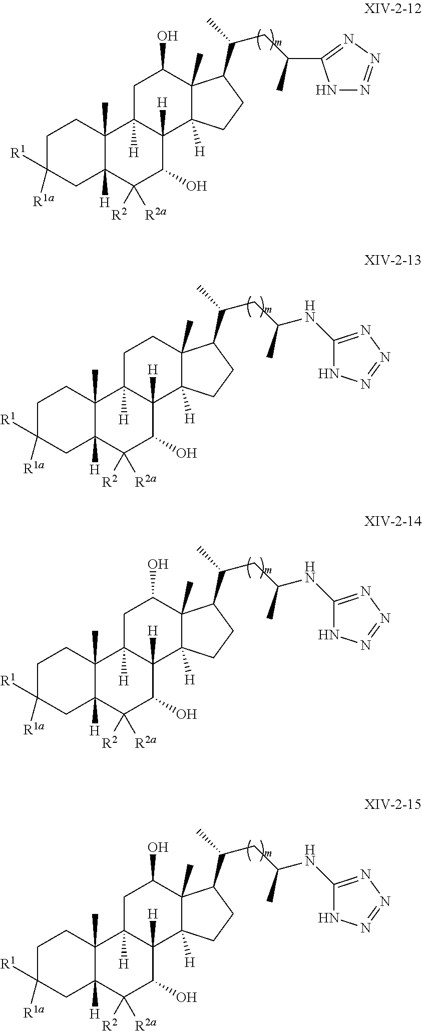

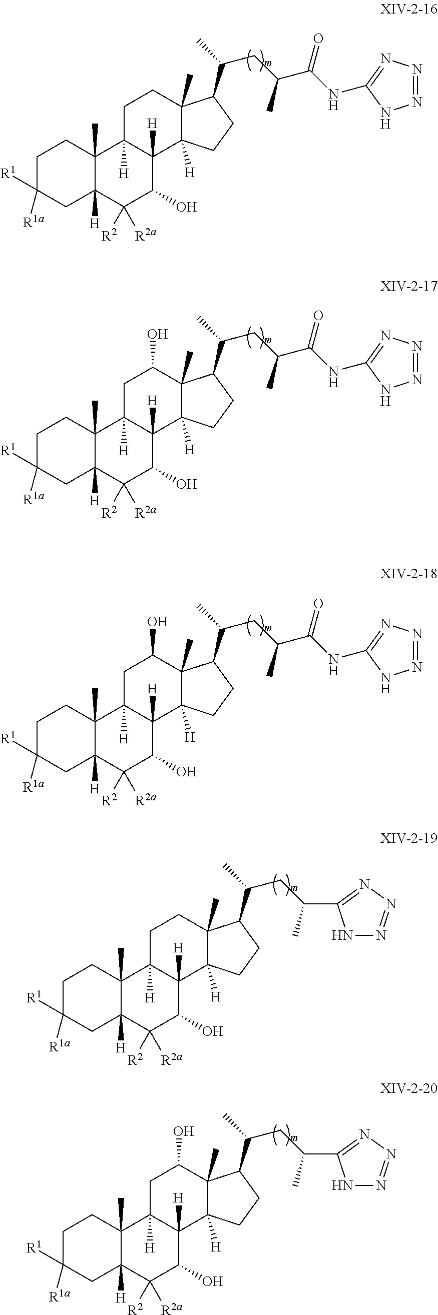

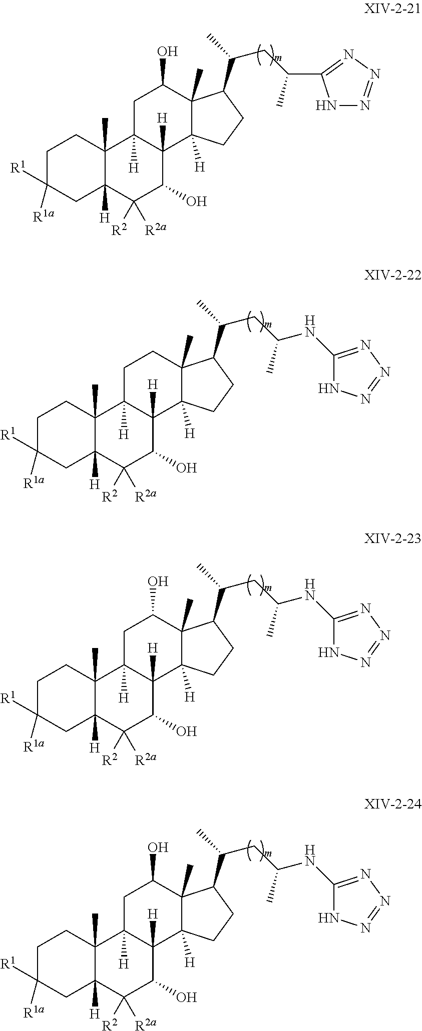

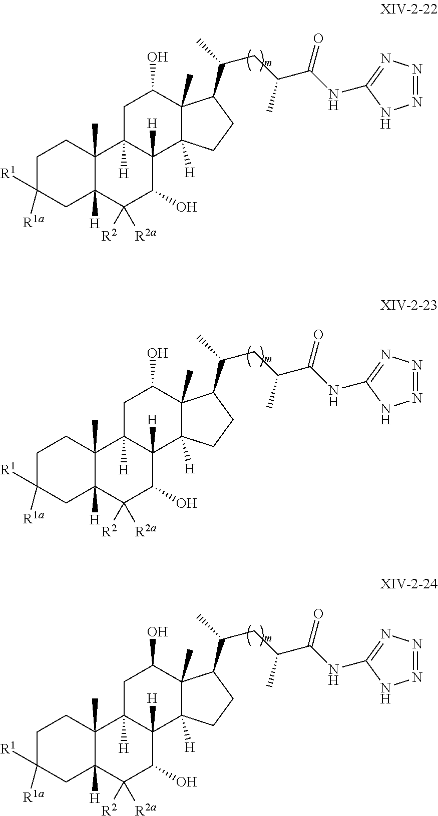

In some embodiments, R^{L} is R₁ as described herein. In some embodiments, L¹ is —CH(CH₃)-L^{b}-Cy- as described herein, wherein -Cy- is bonded to R^{L}. In some embodiments, L¹ is —(R)—CH(CH₃)-L^{b}-Cy- as described herein, wherein -Cy- is bonded to R^{L}. In some embodiments, L¹ is —(S)—CH(CH₃)-L-Cy- as described herein, wherein -Cy- is bonded to R^{L}. In some embodiments, L¹ is —CH(CH₃)-L^{b}-Ar— as described herein, wherein —Ar— is bonded to R^{L}. In some embodiments, L¹ is —(R)—CH(CH₃)-L^{b}-Ar— as described herein, wherein —Ar— is bonded to R^{L}. In some embodiments, L¹ is —(S)—CH(CH₃)-L^{b}-Ar— as described herein, wherein —Ar— is bonded to RLi

In some embodiments, -Cy- is —Ar—. In some embodiments, R⁵ is —H. In some embodiments, R¹³ is methyl. In some embodiments, R^{4a} is —H. In some embodiments, R¹⁴ is R₅ as described herein. In some embodiments, R^{14a} is —H. In some embodiments, R¹⁰ is —H. In some embodiments, R⁹ is methyl. In some embodiments, R⁸ is —H. In some embodiments, R² is R₈ as described herein. In some embodiments, R^{2a} is —H. In some embodiments, R³ is —OR₇ wherein R₇ is as described herein. In some embodiments, R^{3a} is —H. In some embodiments, R¹¹ is —H. In some embodiments, R¹² is —H. In some embodiments, R⁷ is —H. In some embodiments, R^{7a} is —H. In some embodiments, R⁶ is —H. In some embodiments, R^{6a} is —H.

In some embodiments, a provided compound is a compound of formula G-I or a salt thereof:

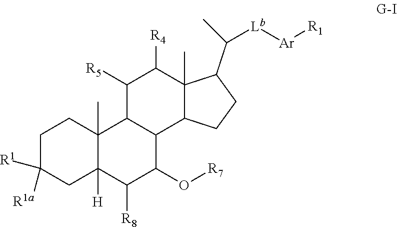

wherein:
   each of R¹ and R^{1a} is independently as described herein;
   R₁ is R₃₀ or R^{s} as described herein;
   —Ar— is an optionally substituted bivalent aryl (e.g., C₆₋₁₄ aryl) or heteroaryl (e.g., 5-14 membered heteroaryl having 1-10 heteroatoms) ring;
   each of R₄, R₅, R₇, and R₈ is independently R^{s} as described herein.

In some embodiments, a provided compound is a compound of formula G-I′ or a salt thereof:

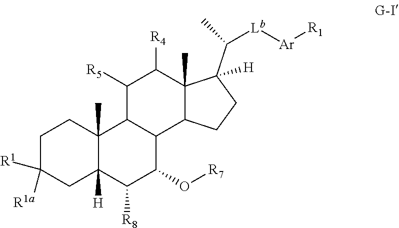

wherein each variable is independently as described herein.

In some embodiments:
R₁ is —H, —C(O)R₁₁, —C(O)NR₁₂R₁₃, —C(O)NHS(O)₂R₁₁, —P(O)(R₁₁)₂, —CN, ortetrazolyl;
Ar is a bivalent optionally substituted aryl (e.g., C₆₋₁₄ aryl) or heteroaryl (e.g., 5-14 membered heteroaryl having 1-10 heteroatoms) ring; preferably, Ar is a bivalent phenyl, pyridyl, pyrimidyl, pyrazinyl, pyridazinyl, triazinyl, tetrazinyl, tetrazolyl, indolyl, indazolyl, thienyl, benzothienyl, furanyl, benzofuranyl, isoxazolyl, benzoxazolyl, thiazolyl, benzothiazolyl, benzotriazolyl, imidazolyl, or benzoimidazolyl ring, each of which is optionally substituted;
L^{b} is an optionally substituted group selected from C₂₋₈ alkylene, C₂₋₈ alkenylene, C₂₋₈ alkynylene, C₃₋₈ cycloalkylene, C₁₋₇ alkoxylene, C₁₋₇ aminoalkylene, C₃₋₇ heterocycloalkylene, C₁₋₈ alkylene-C₃₋₈ cycloalkylene, C₁₋₈ alkylene-C₃₋₇ heterocycloalkylene (e.g., having 1-5 hetereoatoms), bivalent alkylaryl (e.g., C₁₋₈ alkylene-6-14 membered arylene), alkylheteroaryl (e.g., C₁₋₈ alkylene-5-14 membered heteroarylene having 1-5 heteroatoms);
R₄ is —H, —OH, halogen, —OSO₃H, —OAc or —OPO₃H₂; preferably R₄ is —H or —OH; more preferably —H;
R₅ is —H, halogen, —CN, —N₃, —OH, —OSO₃H, —OAc, —OPO₃H₂, —SR₁₂, or —NHR₁₂; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, t-butyldimethylsilyl or benzyl); preferably R₇ is —H;
R₈ is —H, halogen, or an optionally substituted group selected from C₁₋₈ alkyl, C₂₋₈ alkenyl, C₂₋₈ alkynyl, C₃₋₈ cycloalkyl, C₁₋₇ alkoxy, C₁₋₇ aminoalkyl, C₃₋₇ heterocyclyl (e.g., having 1-5 heteroatoms); preferably R₈ is C₁₋₄ alkyl; more preferably R₈ is ethyl;
R₁₁ is —H, halogen, —OH, or an optionally substituted group selected from C₁₋₈ alkoxy, C₁₋₈ alkyl, C₂₋₈ alkenyl, C₂₋₈ alkynyl, C₃₋₈ cycloalkyl, C₃₋₈ cycloalkenyl, C₃₋₈ heterocycloalkyl, aryl (e.g., C₆₋₁₄ aryl), arylalkyl (e.g., C₆₋₁₄arylC₁₋₁₂alkyl), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl); and
each R₁₂ and R₁₃ is independently —H, or an optionally substituted group selected from C₁₋₈ alkyl, C₂₋₈ alkenyl, C₂₋₈ alkynyl, C₃₋₈ cycloalkyl, 3- to 8-membered heterocyclyl (e.g., having 1-5 heteroatoms), aryl (e.g., C₆₋₁₄ aryl), arylalkyl (e.g., C₆₋₁₄arylC₁₋₁₂alkyl), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl); preferably R₁₂ is an optionally substituted group selected from C₁₋₈ alkyl and aryl; and/or preferably R₁₃ is —H; alternatively R₁₂ an R₁₃ are taken together with the nitrogen atom to which they are attached to form an optionally substituted heterocyclic ring (e.g., an optionally substituted 3-15 membered, monocyclic, bicyclic or polycyclic heterocyclyl ring having 1-5 heteroatoms).

In some embodiments, a provided compound is a compound of formula G-IA or a salt thereof:

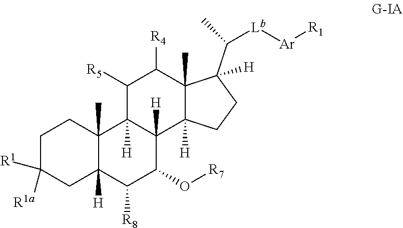

wherein each variable is independently as described herein.

In some embodiments, a provided compound is a compound of formula G-II or a salt thereof:

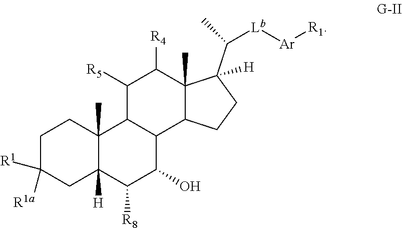

wherein each variable is independently as described herein.

In some embodiments, a provided compound is a compound of formula G-III or a salt thereof:

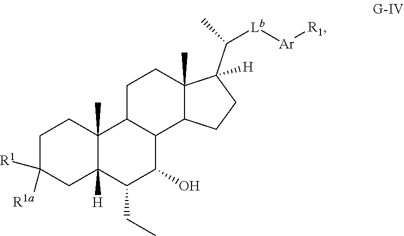

wherein each variable is independently as described herein.

In some embodiments, a provided compound is a compound of formula G-IV or a salt thereof:

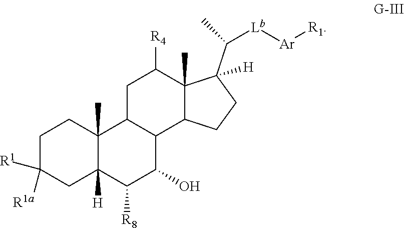

wherein each variable is independently as described herein.

In some embodiments, —Ar—R^{L} is selected from below, wherein —Ar— is optionally substituted (e.g., in the first structure, the phenyl ring is optionally substituted). In some embodiments, —Ar—R₁ is selected from below, wherein —Ar— is optionally substituted (e.g., in the first structure, the phenyl ring is optionally substituted).

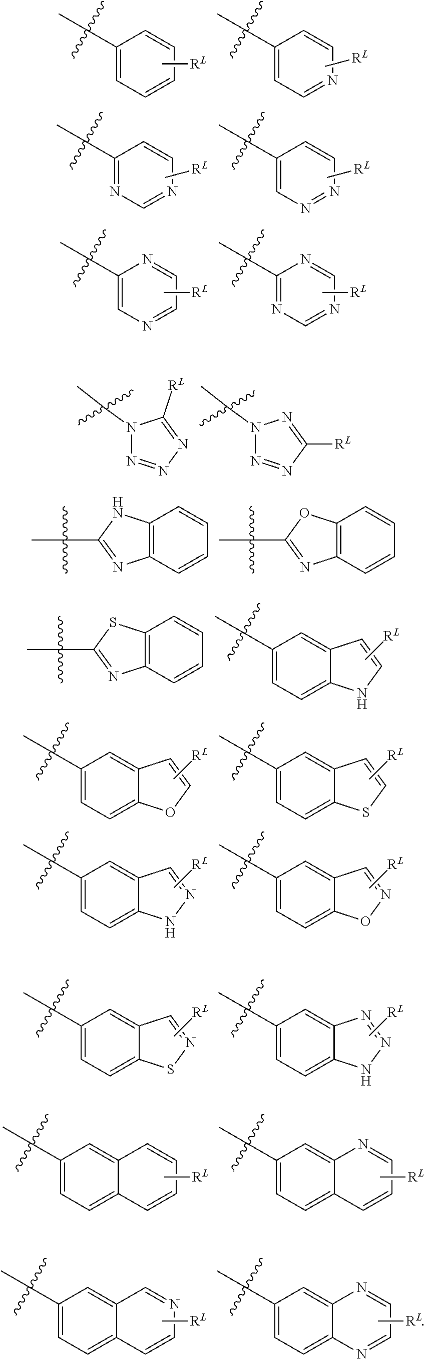

In some embodiments, —Ar— is substituted. In some embodiments, —Ar— is unsubstituted.

In some embodiments, R^{L} is —CO₂R. In some embodiments, R^{L} is —CO₂H. In some embodiments, R^{L} is —CO₂CH₃. In some embodiments, R^{L} is —CO₂Et. In some embodiments, R^{L} is tetrazolyl. In some embodiments, R₁ is —CO₂R. In some embodiments, R₁ is —CO₂H. In some embodiments, R₁ is —CO₂CH₃. In some embodiments, R₁ is —CO₂Et. In some embodiments, R₁ is tetrazolyl. In some embodiments, —Ar—R^{L} is selected from a group below, wherein —Ar— is optionally substituted (e.g., in the first structure, the phenyl ring is optionally substituted). In some embodiments, —Ar—R₁ is selected from a group below, wherein —Ar— is optionally substituted (e.g., in the first structure, the phenyl ring is optionally substituted):

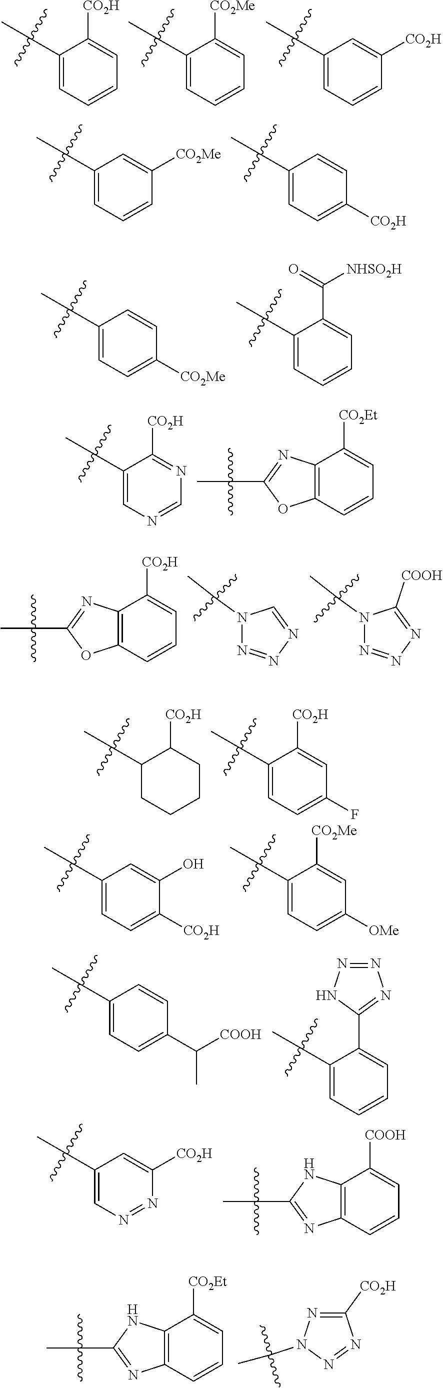

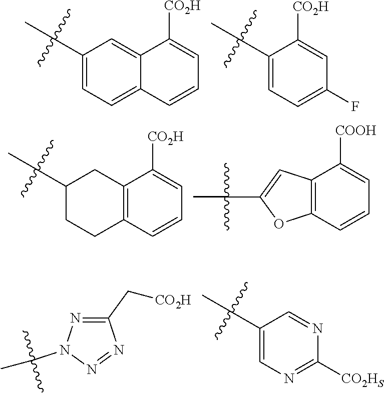

In some embodiments, —Ar— is substituted. In some embodiments, —Ar— is unsubstituted.

In some embodiments, L^{b} is an optionally substituted group selected from below.

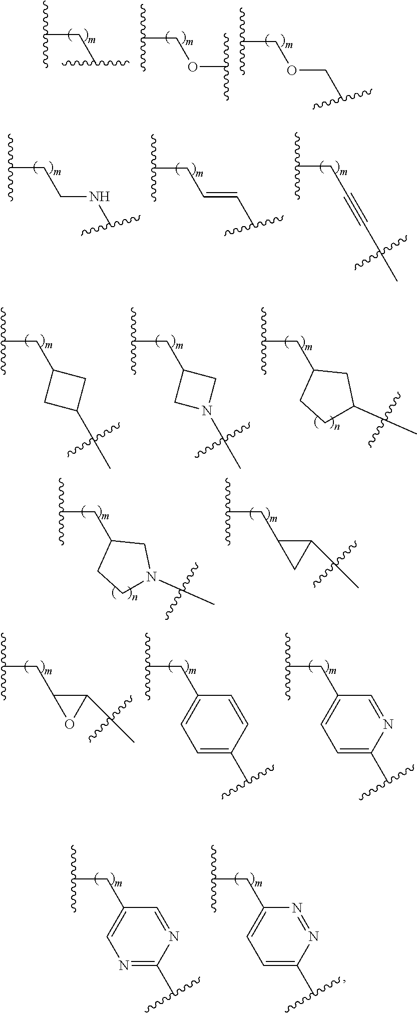

wherein m is 1, 2, 3, 4, 5, 6, or 7; n is 1, 2, or 3; preferably, when present, the optional substituent is —OH, halogen, alkyl, alkoxy, aryl or haloalkyl such as —CF₃. In some embodiments, L is not substituted.

In some embodiments, when L^{b} is C₁₋₄ alkylene, —Ar— is not a bivalent 5-membered heteroaryl group having three heteroatoms selected from oxygen, sulfur and nitrogen, such as bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl. In some embodiments, L^{b} is optionally substituted C₁₋₄ alkylene. In some embodiments, —Ar— is an optionally substituted bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl). In some embodiments, L^{b} is optionally substituted C₁₋₄ alkylene, and —Ar— is an optionally substituted bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl). In some embodiments, L^{b} is C₁₋₄ alkylene, and —Ar— is a bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl).

In some embodiments, when L^{b} is bivalent C₁₋₄ alkylheteroaryl (e.g., having 1-5 heteroatoms), —Ar— is not a bivalent 5-membered heteroaryl group having three heteroatoms selected from oxygen, sulfur and nitrogen, such as bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl. In some embodiments, L^{b} is optionally substituted C₁₋₄ alkylheteroaryl (e.g., having 1-5 heteroatoms). In some embodiments, —Ar— is an optionally substituted bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl). In some embodiments, L^{b} is optionally substituted bivalent C₁₋₄ alkylheteroaryl (e.g., having 1-5 heteroatoms), and —Ar— is an optionally substituted bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl). In some embodiments, L^{b} is bivalent C₁₋₄ alkylheteroaryl (e.g., having 1-5 heteroatoms), and —Ar— is a bivalent 5-membered heteroaryl group having three heteroatoms selected from oxygen, sulfur and nitrogen (e.g., bivalent 1,2,3-triazolyl, 1,3,4-triazolyl, 1,2,3-oxadiazolyl, 1,3,4-oxadiazolyl, 1, 2, 4-oxadiazolyl, 1,2,3-thiadiazolyl, 1,3,4-thiadiazolyl, or 1,2,4-thiadiazolyl).

Various additional embodiments of R₁, R₄, R₅, R₇, and R₈, are described in WO 2017/147137 (e.g., as R₁, R₂, R₃, R₅, and R₆, respectively) either individually or in combination (e.g., as in compounds).

In some embodiments, a provided compound is a compound described in WO 2017/147137, wherein its 3-groups attached to moiety A are replaced with R¹ and R^{1a}, e.g., in some embodiments, both replaced with —H.

In some embodiments, a provided compound is a compound selected from compounds 1-42 of WO 2017/147137, wherein its 3-OH and 3-H attached to moiety A are replaced with R¹ and R^{1a}, e.g., in some embodiments, both replaced with —H.

In some embodiments, a provided compound is a compound selected from compounds in Schemes 1-12, Examples 1-11, and Tables 1-2 of WO 2017/147137, wherein its 3-groups (e.g., 3-OH/protected OH and 3-H attached to moiety A) are replaced with R¹ and R^{1a}, for example, in some embodiments, both replaced with —H.

In some embodiments, a compound is a compound in Table 2 of WO 2017/147137, wherein the compound has an EC50 labeled as “A” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 2 of WO 2017/147137, wherein the compound has an EC50 labeled as “B” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 2 of WO 2017/147137, wherein the compound has an EC50 labeled as “C” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 2 of WO 2017/147137, wherein the compound has an EC50 labeled as “D” and wherein its 3-groups (e.g., 3-groups such as 3-OH/protected OH and 3-H attached to moiety A) are replaced with R¹ and R^{1a}, for example, in some embodiments, both replaced with —H.

As described herein, in some embodiments, one or more isotopes may be utilized or enriched in compounds of the present disclosure at one or more locations. For example, in some embodiments, deuterium is utilized or enriched at one or more positions.

In some embodiments, the present disclosure provides a compound having the structure of VI-I or VI-II, or a salt thereof:

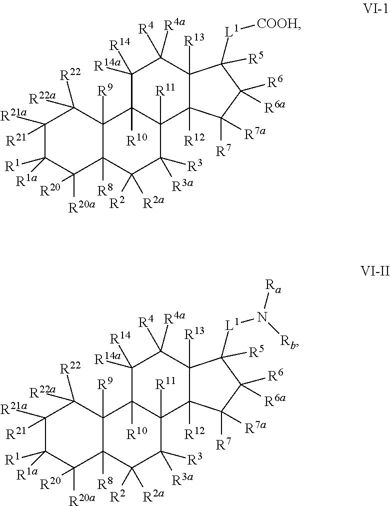

wherein each variable is independently as described herein, and wherein the compound comprises deuterium at one or more locations.

In some embodiments, the present disclosure provides a compound having the structure of H—I or H-II, or a salt thereof:

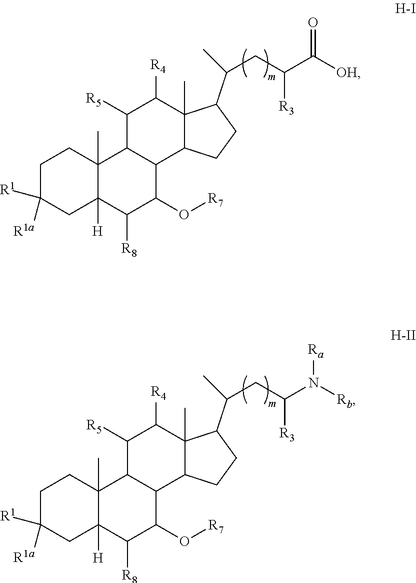

wherein each variable is independently as described herein.

In some embodiments, the present disclosure provides a compound having the structure of H—I′ or H-II′, or a salt thereof:

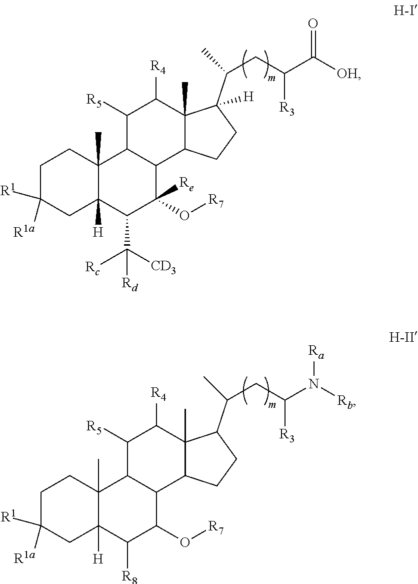

wherein each variable is independently as described herein.

In some embodiments:
Rₐ is —H or optionally substituted C₁₋₈ alkyl; preferably Rₐ is —H or methyl; more preferably Rₐ is —H;
R_{b} is —H, —C(O)NR₉R₁₀, —C(O)NHS(O)₂R₁ or —S(O)₂R₁;
R_{c} is ¹H or D (deuterium);
R_{d} is ¹H or D;
Rₑ is ¹H or D;
R₁ is halogen, —OH, —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), heterocyclyl (e.g., 3-15 membered heterocyclyl having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl);
R₃ is —H or an optionally substituted group selected from C₁₋₈ alkyl, C₂₋₈ alkenyl, C₂₋₈ alkynyl, aryl (e.g., C₆₋₁₄ aryl), and arylalkyl (e.g., C₆₋₁₄arylC₁₋₁₂alkyl); preferably R₃ is —H or methyl;
m is 0, 1, 2, or 3; preferably m is 0, 1, or 2;
R₄ is —H, —OH, —OSO₃H, —OAc or —OPO₃H₂; preferably R₄ is —H;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H; and
R₉ and R₁₀ are each independently —H, or an optionally substituted group selected from C₁₋₈ alkyl, C₂₋₈ alkenyl, C₂₋₈ alkynyl, C₃₋₈ cycloalkyl, and heterocyclyl (e.g., 3-15 membered heterocyclyl having 1-5 heteroatoms), or R₉ and R₁₀ are taken together with the nitrogen to which they are attached to form an optionally substituted 3-15 membered, monocyclic, bicyclic or polycyclic heterocyclyl ring having 1-5 heteroatoms; preferably R₁₀ is —H.

In some embodiments, the present disclosure provides a compound having the structure of H-IA or H-IIA, or a salt thereof:

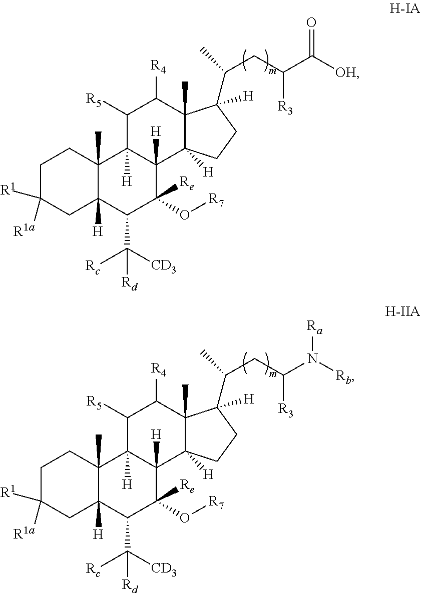

wherein each variable is independently as described herein.

In some embodiments, the present disclosure provides a compound having the structure of H—III or H—IV, or a salt thereof:

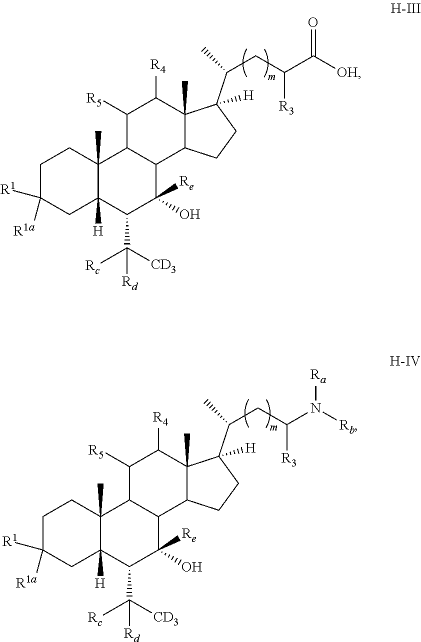

wherein each variable is independently as described herein.

In some embodiments, the present disclosure provides a compound having the structure of H—V or H—VI, or a salt thereof:

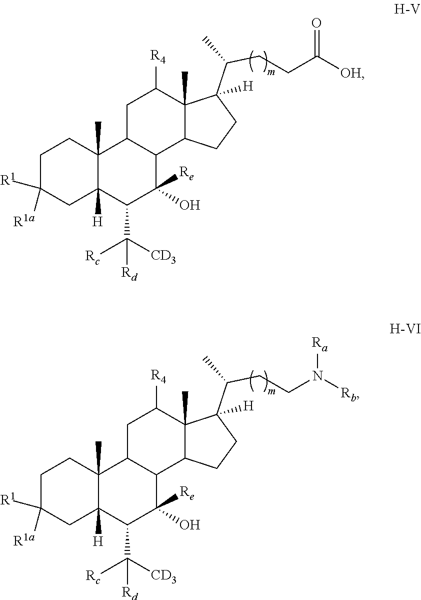

wherein each variable is independently as described herein.

In some embodiments, the present disclosure provides a compound having the structure of H-VII or H-VIII, or a salt thereof:

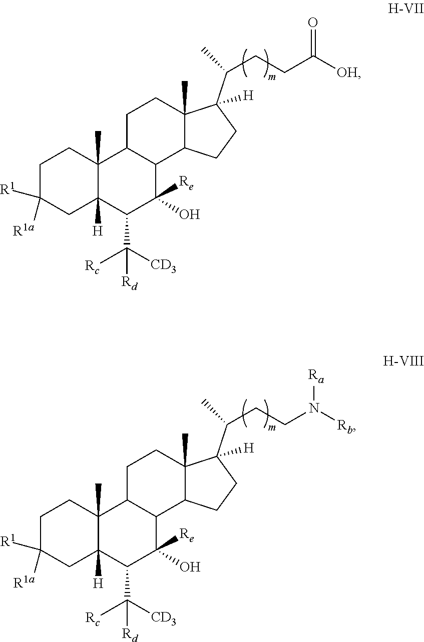

wherein each variable is independently as described herein.

In some embodiments, the present disclosure provides a compound having the structure of H-IX or H-X, or a salt thereof:

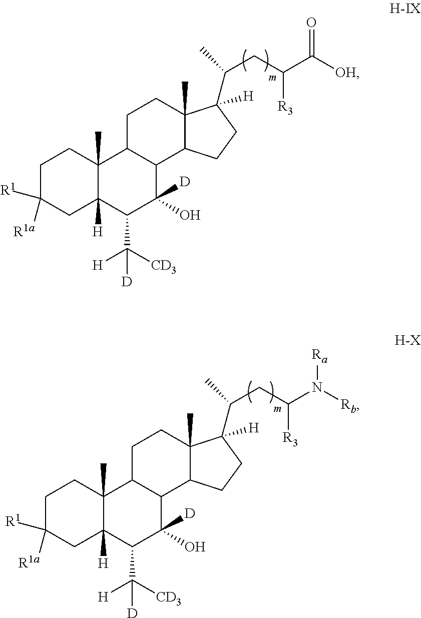

wherein each variable is independently as described herein.

In some embodiments, the present disclosure provides a compound having the structure of H-H-X-A or H-X-B, or a salt thereof:

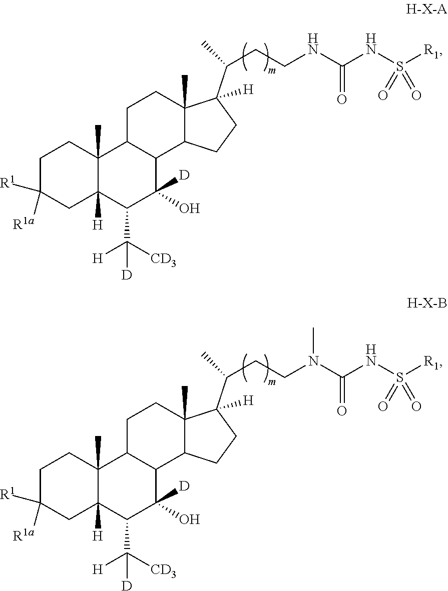

wherein each variable is independently as described herein.

In some embodiments, the present disclosure provides a compound having the structure of H-H-XI-A or H-XI-B, or a salt thereof:

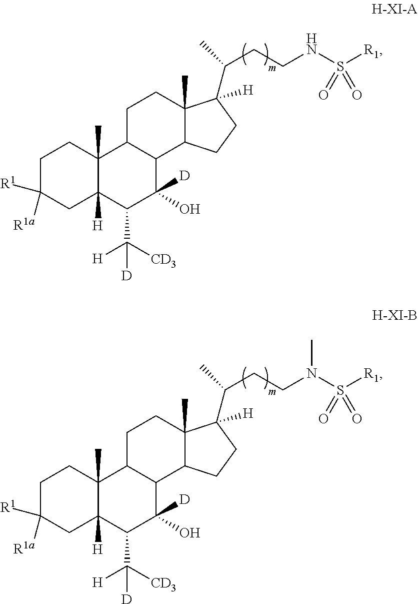

wherein each variable is independently as described herein.

In some embodiments, the present disclosure provides a compound having the structure of H-H-XII-A or H-XII-B, or a salt thereof:

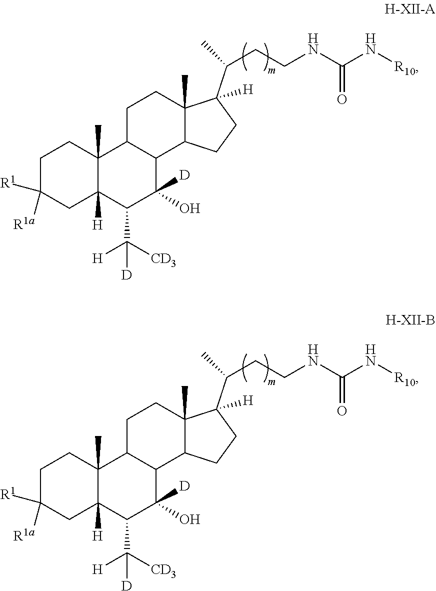

wherein each variable is independently as described herein.

In some embodiments, a compound has a structure selected below or a salt thereof, wherein each variable is independently as described herein.

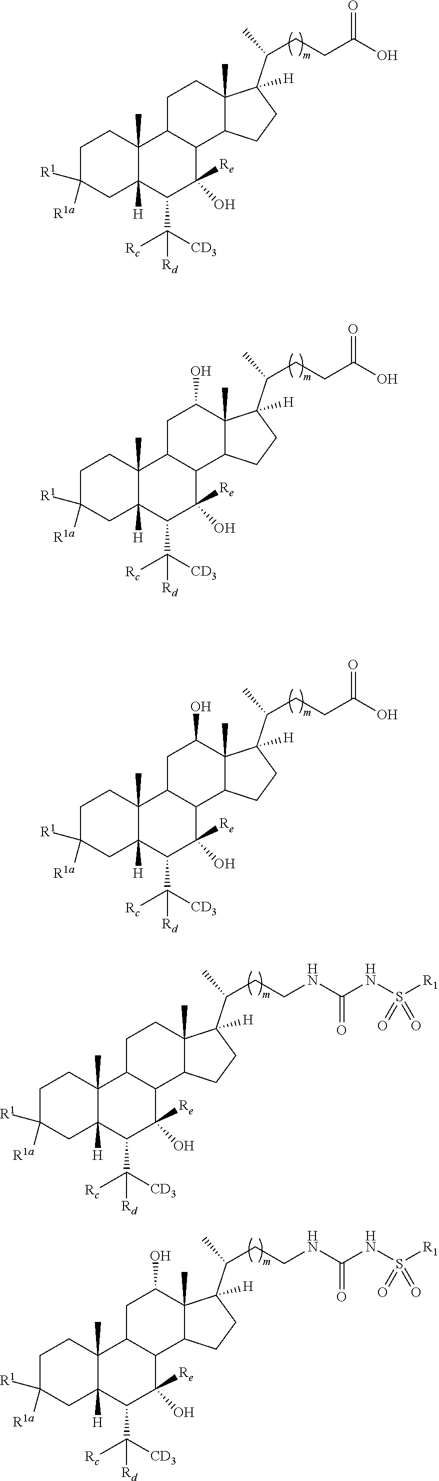

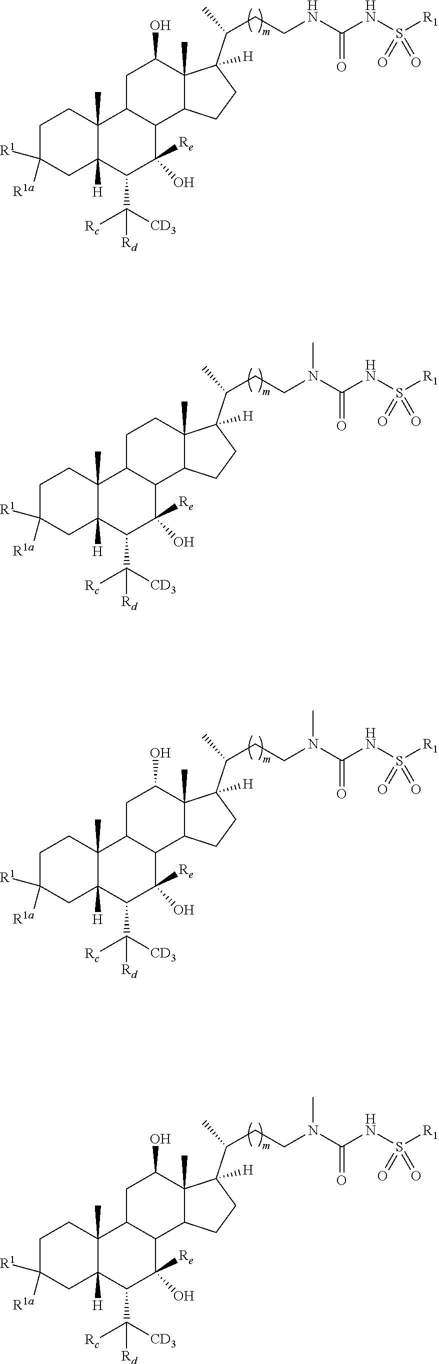

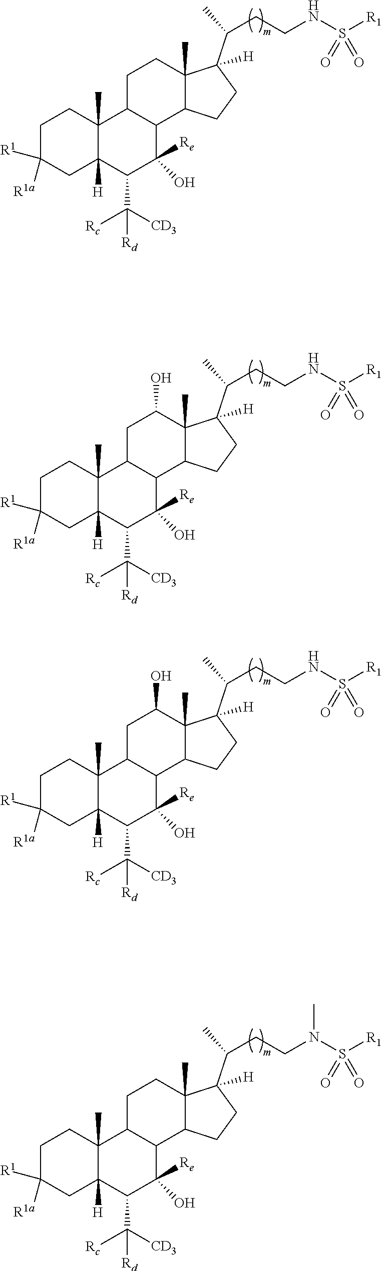

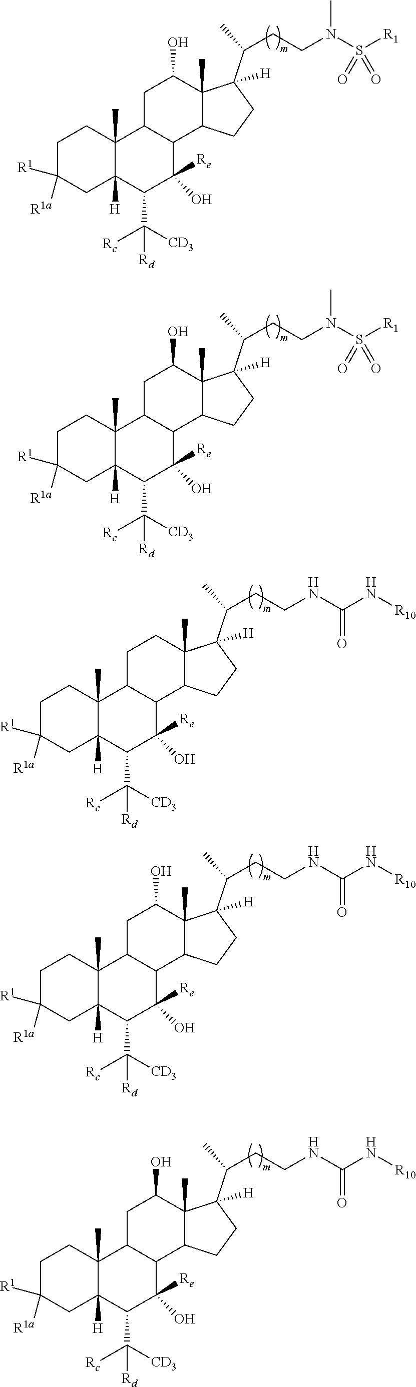

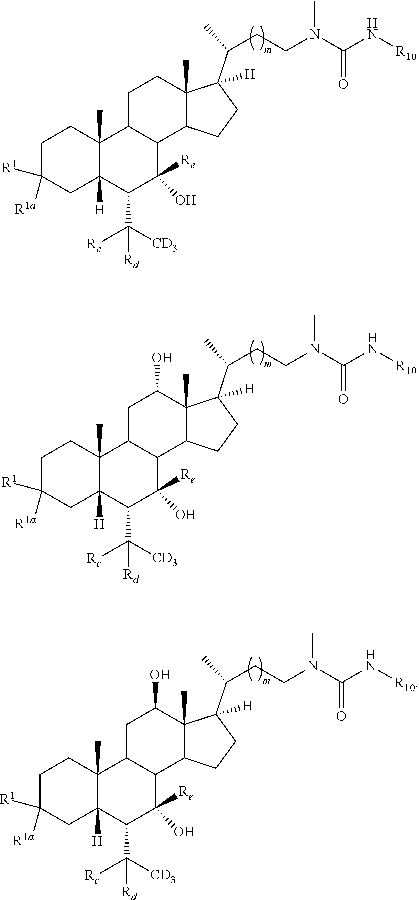

In some embodiments, R₁ is optionally substituted C₁₋₄ alkyl. In some embodiments, R₁ is C₁₋₄ alkyl. In some embodiments, R₁ is halogenated C₁₋₄ alkyl. In some embodiments, R₁ is optionally substituted C₁₋₄ alkenyl. In some embodiments, R₁ is C₁₋₄ alkenyl. In some embodiments, R₁ is phenyl-C₁ 4 alkyl. In some embodiments, R₁ is optionally substituted C₃₋₆ cycloalkyl. In some embodiments, R₁ is C₃₋₆ cycloalkyl. In some embodiments, R₁ is C₃₋₆ cycloalkyl-C₁₋₄ alkyl. In some embodiments, R₁ is optionally substituted 5- or 6-membered heterocycloalkyl (e.g., having 1-4 heteroatoms). In some embodiments, R₁ is 5- or 6-membered heterocycloalkyl (e.g., having 1-4 heteroatoms). In some embodiments, R₁ is amino. In some embodiments, R₁ is optionally substituted phenyl. In some embodiments, R₁ is phenyl. In some embodiments, R₁ is halogen.

In some embodiments, R¹ is —H. In some embodiments, R¹ is —F. In some embodiments, R^{1a} is —H. In some embodiments, R^{1a} is —F. In some embodiments, R¹ and R^{1a} are —H. In some embodiments, one of R¹ and R^{1a} is —F and the other is —H. In some embodiments, R¹ and R^{1a} are —F.

In some embodiments, R₁ is optionally substituted C₁₋₆ alkyl. In some embodiments, R₁ is methyl. In some embodiments, R₁ is ethyl. In some embodiments, R₁ is butyl. In some embodiments, R₁ is t-butyl. In some embodiments, R₁ is propyl. In some embodiments, R₁ is isopropyl. In some embodiments, R₁ is benzyl. In some embodiments, R₁ is vinyl. In some embodiments, R₁ is ally. In some embodiments, R₁ is —CF₃. In some embodiments, R₁ is cyclopropyl. In some embodiments, R₁ is 1-methylcyclopropyl. In some embodiments, R₁ is cyclopropylmethyl. In some embodiments, R₁ is 1-pyrrolidinyl. In some embodiments, R₁ is 1-piperidinyl. In some embodiments, R₁ is 4-morpholinyl. In some embodiments, R₁ is —NH₂. In some embodiments, R₁ is dimethylamino. In some embodiments, R₁ is optionally substituted phenyl. In some embodiments, R₁ is phenyl. In some embodiments, R₁ is 4-trifluoromethoxyphenyl. In some embodiments, R₁ is 2-methylphenyl. In some embodiments, R₁ is 2-trifluoromethoxyphenyl. In some embodiments, R₁ is 2-fluorophenyl. In some embodiments, R₁ is 4-tert-butylphenyl. In some embodiments, R₁ is 2-naphthyl. In some embodiments, R₁ is 4-methylphenyl. In some embodiments, R₁ is 2-methoxyphenyl. In some embodiments, R₁ is —F.

Certain embodiments of R^{L} are described in Table 6 below. In some embodiments, R₁ is as described in Table 6 below.

TABLE 6
Certain examples of m and R^{L}/R₁.

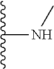 [SMILES: *NC]

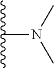 [SMILES: *N(C)C]

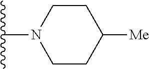 [SMILES: *N1CCC(C)CC1]

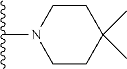 [SMILES: *N1CCC(C)(C)CC1]

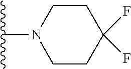 [SMILES: *N1CCC(F)(F)CC1]

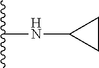 [SMILES: *NC1CC1]

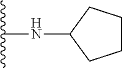 [SMILES: *NC1CCCC1]

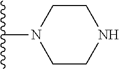 [SMILES: *N1CCNCC1]

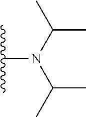 [SMILES: *N(C(C)C)C(C)C]

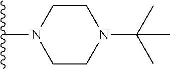 [SMILES: *N1CCN(C(C)(C)C)CC1]

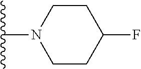 [SMILES: *N1CCC(F)CC1]

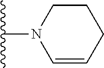 [SMILES: *N1C=CCCC1]

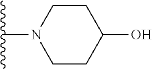 [SMILES: *N1CCC(O)CC1]

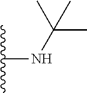 [SMILES: *NC(C)(C)C]

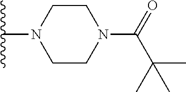 [SMILES: *N1CCN(C(=O)C(C)(C)C)CC1]

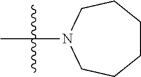 [SMILES: *N1CCCCCC1]

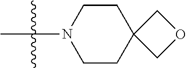 [SMILES: *N1CCC2(CC1)COC2]

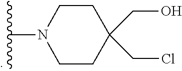 [SMILES: *N1CCC(CO)(CCl)CC1]

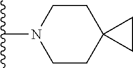 [SMILES: *N1CCC2(CC1)CC2]

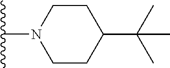 [SMILES: *N1CCC(C(C)(C)C)CC1]

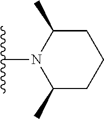 [SMILES: *N1[C@H](C)CCC[C@@H]1C]

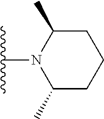 [SMILES: *N1[C@@H](C)CCC[C@@H]1C]

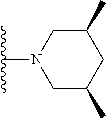 [SMILES: *N1C[C@H](C)C[C@H](C)C1]

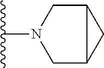 [SMILES: *N1CC2CC2C1]

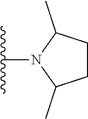 [SMILES: *N1C(C)CCC1C]

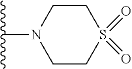 [SMILES: *N1CCS(=O)(=O)CC1]

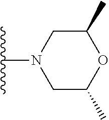 [SMILES: *N1C[C@@H](C)O[C@H](C)C1]

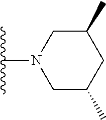 [SMILES: *N1C[C@@H](C)C[C@H](C)C1]

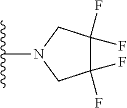 [SMILES: *N1CC(F)(F)C(F)(F)C1]

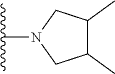 [SMILES: *N1CC(C)C(C)C1]

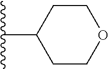 [SMILES: *C1CCOCC1]

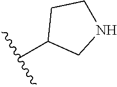 [SMILES: *C1CCNC1]

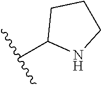 [SMILES: *C1CCCN1]

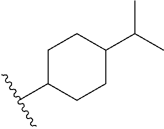 [SMILES: *C1CCC(C(C)C)CC1]

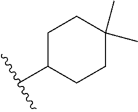 [SMILES: *C1CCC(C)(C)CC1]

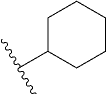 [SMILES: *C1CCCCC1]

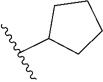 [SMILES: *C1CCCC1]

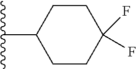 [SMILES: *C1CCC(F)(F)CC1]

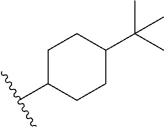 [SMILES: *C1CCC(C(C)(C)C)CC1]

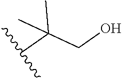 [SMILES: *C(C)(C)CO]

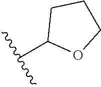 [SMILES: *C1CCCO1]

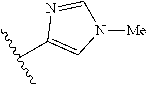 [SMILES: *c1cn(C)cn1]

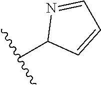 [SMILES: *C1C=CC=N1]

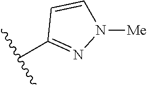 [SMILES: *c1ccn(C)n1]

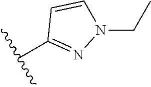 [SMILES: *c1ccn(CC)n1]

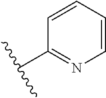 [SMILES: *c1ccccn1]

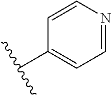 [SMILES: *c1ccncc1]

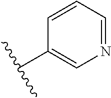 [SMILES: *c1cccnc1]

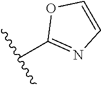 [SMILES: *c1ncco1]

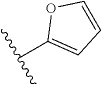 [SMILES: *c1ccco1]

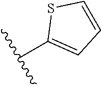 [SMILES: *c1cccs1]

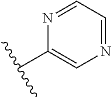 [SMILES: *c1cnccn1]

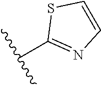 [SMILES: *c1nccs1]

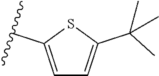 [SMILES: *c1ccc(C(C)(C)C)s1]

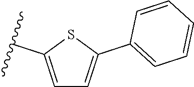 [SMILES: *c1ccc(-c2ccccc2)s1]

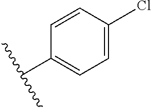 [SMILES: *c1ccc(Cl)cc1]

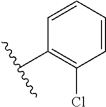 [SMILES: *c1ccccc1Cl]

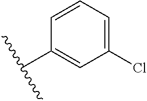 [SMILES: *c1cccc(Cl)c1]

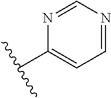 [SMILES: *c1ccncn1]

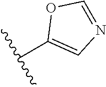 [SMILES: *c1cnco1]

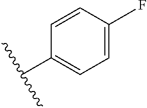 [SMILES: *c1ccc(F)cc1]

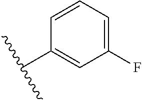 [SMILES: *c1cccc(F)c1]

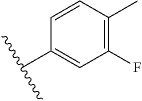 [SMILES: *c1ccc(C)c(F)c1]

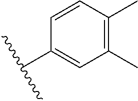 [SMILES: *c1ccc(C)c(C)c1]

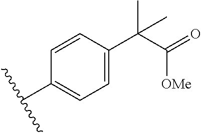 [SMILES: *c1ccc(C(C)(C)C(=O)OC)cc1]

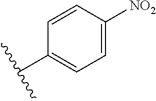 [SMILES: *c1ccc([N+](=O)[O-])cc1]

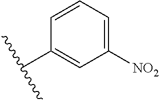 [SMILES: *c1cccc([N+](=O)[O-])c1]

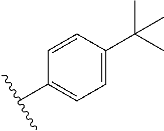 [SMILES: *c1ccc(C(C)(C)C)cc1]

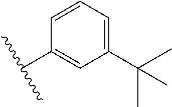 [SMILES: *c1cccc(C(C)(C)C)c1]

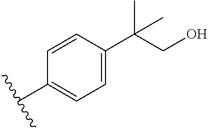 [SMILES: *c1ccc(C(C)(C)CO)cc1]

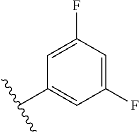 [SMILES: *c1cc(F)cc(F)c1]

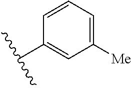 [SMILES: *c1cccc(C)c1]

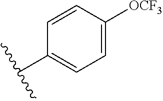 [SMILES: *c1ccc(OC(F)(F)F)cc1]

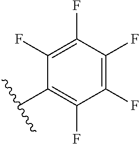 [SMILES: *c1c(F)c(F)c(F)c(F)c1F]

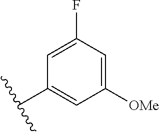 [SMILES: *c1cc(F)cc(OC)c1]

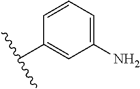 [SMILES: *c1cccc(N)c1]

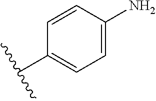 [SMILES: *c1ccc(N)cc1]

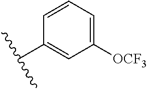 [SMILES: *c1cccc(OC(F)(F)F)c1]

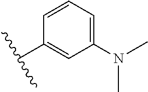 [SMILES: *c1cccc(N(C)C)c1]

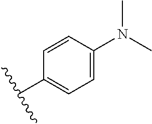 [SMILES: *c1ccc(N(C)C)cc1]

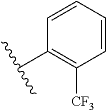 [SMILES: *c1ccccc1C(F)(F)F]

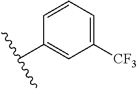 [SMILES: *c1cccc(C(F)(F)F)c1]

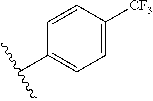 [SMILES: *c1ccc(C(F)(F)F)cc1]

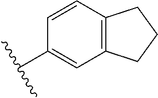 [SMILES: *c1ccc2c(c1)CCC2]

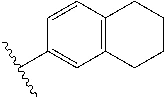 [SMILES: *c1ccc2c(c1)CCCC2]

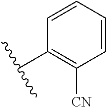 [SMILES: *c1ccccc1C#N]

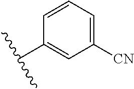 [SMILES: *c1cccc(C#N)c1]

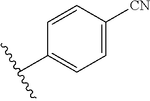 [SMILES: *c1ccc(C#N)cc1]

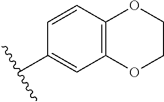 [SMILES: *c1ccc2c(c1)OCCO2]

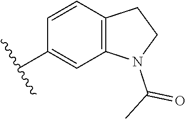 [SMILES: *c1ccc2c(c1)N(C(C)=O)CC2]

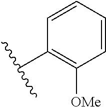 [SMILES: *c1ccccc1OC]

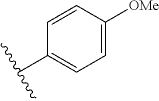 [SMILES: *c1ccc(OC)cc1]

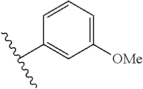 [SMILES: *c1cccc(OC)c1]

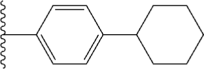 [SMILES: *c1ccc(C2CCCCC2)cc1]

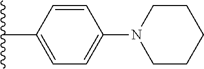 [SMILES: *c1ccc(N2CCCCC2)cc1]

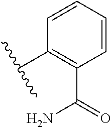 [SMILES: *c1ccccc1C(N)=O]

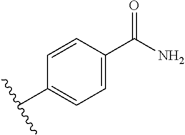 [SMILES: *c1ccc(C(N)=O)cc1]

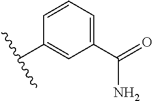 [SMILES: *c1cccc(C(N)=O)c1]

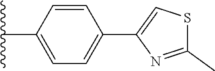 [SMILES: *c1ccc(-c2csc(C)n2)cc1]

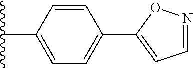 [SMILES: *c1ccc(-c2ccno2)cc1]

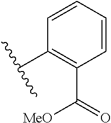 [SMILES: *c1ccccc1C(=O)OC]

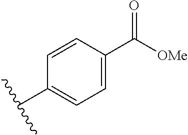 [SMILES: *c1ccc(C(=O)OC)cc1]

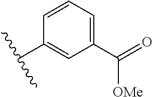 [SMILES: *c1cccc(C(=O)OC)c1]

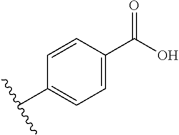 [SMILES: *c1ccc(C(=O)O)cc1]

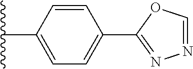 [SMILES: *c1ccc(-c2nnco2)cc1]

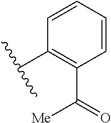 [SMILES: *c1ccccc1C(C)=O]

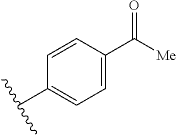 [SMILES: *c1ccc(C(C)=O)cc1]

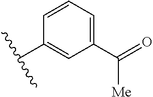 [SMILES: *c1cccc(C(C)=O)c1]

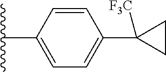 [SMILES: *c1ccc(C2(C(F)(F)F)CC2)cc1]

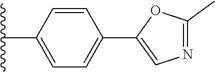 [SMILES: *c1ccc(-c2cnc(C)o2)cc1]

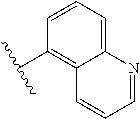 [SMILES: *c1cccc2ncccc12]

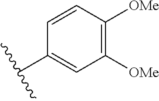 [SMILES: *c1ccc(OC)c(OC)c1]

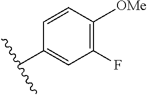 [SMILES: *c1ccc(OC)c(F)c1]

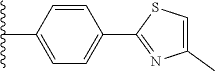 [SMILES: *c1ccc(-c2nc(C)cs2)cc1]

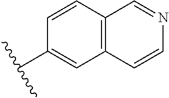 [SMILES: *c1ccc2cnccc2c1]

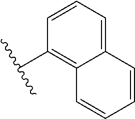 [SMILES: *c1cccc2ccccc12]

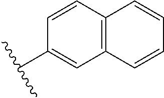 [SMILES: *c1ccc2ccccc2c1]

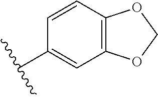 [SMILES: *c1ccc2c(c1)OCO2]

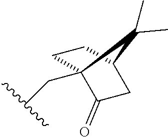

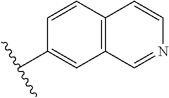 [SMILES: *c1ccc2ccncc2c1]

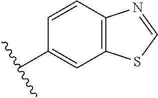 [SMILES: *c1ccc2ncsc2c1]

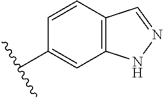 [SMILES: *c1ccc2cn[nH]c2c1]

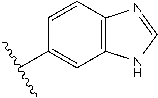 [SMILES: *c1ccc2nc[nH]c2c1]

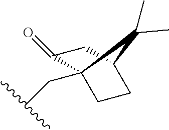

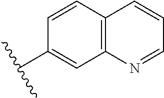 [SMILES: *c1ccc2cccnc2c1]

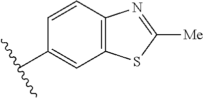 [SMILES: *c1ccc2nc(C)sc2c1]

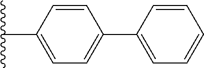 [SMILES: *c1ccc(-c2ccccc2)cc1]

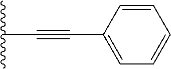 [SMILES: *C#Cc1ccccc1]

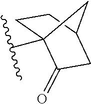 [SMILES: *C12CCC(CC1=O)C2]

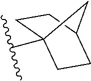 [SMILES: *C12CCC(CC1)C2]

In some embodiments, R₁₀ is —H and R₉ is —H, C₁₋₄ alkyl, C₁₋₄ haloalkyl, C₂₋₄ alkenyl, phenyl-C₁₋₄ alkyl, optionally substituted C₃₋₅ cycloalkyl, C₃₋₆ cycloalkyl-C₁₋₄ alkyl, 5- or 6-membered heterocycloalkyl (e.g., having 1-5 heteroatoms) or optionally substituted phenyl.

In some embodiments, R¹⁰ is —H and R₉ is —H, methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, phenyl, 4-trifluoromethoxyphenyl, 2-methylphenyl, 2-trifluoromethoxyphenyl, 2-fluorophenyl, 2-naphthyl, 4-methylphenyl, or 2-methoxyphenyl.

In some embodiments, m is 0, R_{c} is ¹H, R_{d} is ¹H, and Rₑ is ¹H. In some embodiments, m is 0, R_{c} is D, R_{d} is ¹H, and Rₑ is ¹H. In some embodiments, m is 0, R_{c} is D, R_{d} is D, and Rₑ is ¹H. In some embodiments, m is 0, R_{c} is ¹H, R_{d} is ¹H, and Rₑ is D. In some embodiments, m is 0, R_{c} is D, R_{d} is ¹H, and Rₑ is D. In some embodiments, m is 0, R_{c} is D, R_{d} is D, and Rₑ is D.

In some embodiments, m is 1, R_{c} is ¹H, R_{d} is ¹H, and Rₑ is ¹H. In some embodiments, m is 1, R_{c} is D, R_{d} is ¹H, and Rₑ is ¹H. In some embodiments, m is 1, R_{c} is D, R_{d} is D, and Rₑ is ¹H. In some embodiments, m is 1, R_{c} is ¹H, R_{d} is ¹H, and Rₑ is D. In some embodiments, m is 1, R_{c} is D, R_{d} is ¹H, and Rₑ is D. In some embodiments, m is 1, R_{c} is D, R_{d} is D, and Rₑ is D.

In some embodiments, m is 2, R_{c} is ¹H, R_{d} is ¹H, and Rₑ is ¹H. In some embodiments, m is 2, R_{c} is D, R_{d} is ¹H, and Rₑ is ¹H. In some embodiments, m is 2, R_{c} is D, R_{d} is D, and Rₑ is ¹H. In some embodiments, m is 2, R_{c} is ¹H, R_{d} is ¹H, and Rₑ is D. In some embodiments, m is 2, R_{c} is D, R_{d} is ¹H, and Rₑ is D. In some embodiments, m is 2, R_{c} is D, R_{d} is D, and Rₑ is D.

In some embodiments, m is 0, and R₁ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F. In some embodiments, m is 1, and R₁ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F. In some embodiments, m is 2, and R₁ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F.

In some embodiments, m is 0, and R₁₀ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F. In some embodiments, m is 1, and R₁₀ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F. In some embodiments, m is 2, and R₁₀ is methyl, ethyl, isopropyl, butyl, t-butyl, propyl, benzyl, vinyl, ally, —CF₃, cyclopropyl, 1-methylcyclopropyl, cyclopropylmethyl, 1-pyrrolidinyl, 1-piperidinyl, 4-morpholinyl, —NH₂, dimethylamino, phenyl, 4-trifluoromethoxyphenyl, 2-methylphenyl, 2-trifluoromethoxyphenyl, 2-fluorophenyl, 4-tert-butylphenyl, 2-naphthyl, 4-methylphenyl or —F.

As described herein, an isotope at a position may be enriched. In some embodiments, an enrichment is about or at least about 5%, 10%, 15%, 20%, 25%, 30%, 35%, 40%, 45%, 50%, 60%, 70%, 80%, 85%, 90%, 95%, or 99% more than a natural abundance as applicable. In some embodiments, a level of an isotope at a position is about or at least about 5%, 10%, 15%, 20%, 25%, 30%, 35%, 40%, 45%, 50%, 60%, 70%, 80%, 85%, 90%, 95%, or 99% of all compound molecules. For example, in some embodiments, designation of an atom as deuterium indicates that for that atom, at least 5% of all compound molecules are deuterated. In some embodiments, about or at least about 10%, 20%, 30%, 40% or 50% all compound molecules are deuterated at designated positions. In some embodiments, a percentage is about or at least about 60%, 70%, 80%, 85%, 90%, 95%, or 99% of all compound molecules. In some embodiments, a compound may have two or more positions deuterated, each of which independently has a percentage of about or at least about 5%, 10%, 15%, 20%, 25%, 30%, 35%, 40%, 45%, 50%, 60%, 70%, 80%, 85%, 90%, 95%, or 99% of all compound molecules.

Various additional embodiments of R₁, R₃, R₄, R₅, R₇, Rₐ and R_{b} are described in WO 2017/147159 (e.g., as R₁, R₂, R₃, R₄, R₆, Rₐ and R_{b}, respectively) either individually or in combination (e.g., as in compounds).

In some embodiments, a provided compound is a compound described in WO 2017/147159, wherein its 3-groups attached to moiety A are replaced with R¹ and R^{1a}, e.g., in some embodiments, both replaced with —H.

In some embodiments, a provided compound is a compound selected from compounds 1-459 of WO 2017/147159, wherein its 3-OH and 3-H attached to moiety A are replaced with R¹ and R^{1a}, e.g., in some embodiments, both replaced with —H.

In some embodiments, a provided compound is a compound selected from compounds in Schemes 1-4, Examples 1-4, and Table 8 of WO 2017/147159, wherein its 3-groups (e.g., 3-OH/protected OH and 3-H attached to moiety A) are replaced with R¹ and R^{1a}, for example, in some embodiments, both replaced with —H.

In some embodiments, a compound is a compound in Table 8 of WO 2017/147159, wherein the compound has an EC50 labeled as “A” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 2 of WO 2017/147159, wherein the compound has an EC50 labeled as “B” and wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2017/147174, e.g., a compound of formula (I) or a salt thereof (including stereoisomer, solvate, hydrate or combination thereof) as described in WO 2017/147174, wherein —OR₄ in WO 2017/147174 (in various embodiments, R₄ is —OH in WO 2017/147174) is replaced with R¹ or R^{1a} as described herein. In some embodiments, —OR₄ in WO 2017/147174 and the —H that is attached to the same carbon as —OR₄ are replaced with R¹ and R^{1a} as described herein.

In some embodiments, one of R² and R^{2a} is —H and the other is Rₛ as described herein (e.g., in some embodiments, Rₛ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R is optionally substituted C₁₋₆ aliphatic. In some embodiments, R is optionally substituted C₁₋₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{a}-L^{b}-L^{c}-, wherein L^{a} is L^{s0} as described herein, L^{c} is -Cy- as described herein, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L¹ is -L^{a}-L^{b}-L^{c}-, wherein L^{a} is L^{s0} as described herein, L^{c} is a covalent bond, —C(O)N(R′)—, or —N(R′)—, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, a methylene unit bonded to L^{c} is replaced with —C(R′)₂—. In some embodiments, a methylene unit bonded to L^{c} is replaced with —CHR′—. In some embodiments, R′ is R₃ as described herein. In some embodiments, L^{b} is —(CH₂)m-CH(R′)— as described herein. In some embodiments, L^{b} is —(CH₂)m-CH(R₃)— as described herein. In some embodiments, —(CH₂)m- is bonded to L^{a}. In some embodiments, L^{c} is -Cy- wherein -Cy- is an optionally substituted bivalent aromatic ring. In some embodiments, -Cy- is optionally substituted phenylene. In some embodiments, -Cy- is optionally substituted 5-14 membered heteroarylene having 1-10 (e.g., 1, 2, 3, 4, 5, 6, 7, 8, 9, or 10) heteroatoms. In some embodiments, -Cy- is optionally substituted 5-membered heteroarylene having 1-4 (e.g., 1, 2, 3 or 4) heteroatoms. In some embodiments, -Cy- is optionally substituted 5-membered heteroarylene having 1 heteroatom. In some embodiments, -Cy- is optionally substituted 5-membered heteroarylene having 2 heteroatoms. In some embodiments, -Cy- is optionally substituted 5-membered heteroarylene having 3 heteroatoms. In some embodiments, -Cy- is optionally substituted 5-membered heteroarylene having 3 heteroatoms each independently selected from nitrogen, oxygen ad sulfur. In some embodiments, -Cy- has an oxygen or sulfur ring atom and two nitrogen ring atoms. In some embodiments, -Cy- has three nitrogen ring atoms. In some embodiments, -Cy- is optionally substituted 6-membered heteroarylene having 1-4 (e.g., 1, 2, 3 or 4) heteroatoms. In some embodiments, -Cy- is optionally substituted 9-membered bicyclic heteroarylene having 1-6 (e.g., 1, 2, 3, 4, 5 or 6) heteroatoms. In some embodiments, -Cy-is optionally substituted 10-membered bicyclic heteroarylene having 1-6 (e.g., 1, 2, 3, 4, 5 or 6) heteroatoms. In some embodiments, -Cy- is an optionally substituted bivalent phenyl, pyridyl, pyrimidyl, pyrazinyl, pyridazinyl, triazinyl, tetrazinyl, tetrazolyl, indolyl, indazolyl, thienyl, benzothienyl, furanyl, benzofuranyl, isoxazolyl, benzoxazolyl, thiazolyl, benzothiazolyl, benzotriazolyl, imidazolyl, or benzoimidazolyl ring. In some embodiments, L^{c} is a covalent bond. In some embodiments, L^{c} is —N(R′)—. In some embodiments, L^{c} is —NH—. In some embodiments, L^{c} is —C(O)N(R′)—. In some embodiments, L^{c} is —C(O)NH—.

In some embodiments, R^{L} is R^{s}. In some embodiments, R^{L} is R′. In some embodiments, R^{L} is R. In some embodiments, R^{L} is —H. In some embodiments, R^{L} is —C(O)R^{s}. In some embodiments, R^{L} is —C(O)N(R^{s})₂. In some embodiments, R^{L} is —C(O)N(R′)S(O)₂R^{s}. In some embodiments, R^{L} is —P(O)(R^{s})₂. In some embodiments, R^{L} is —CN.

In some embodiments, R^{L} is —H. In some embodiments, R^{L} is optionally substituted heteroaryl. In some embodiments, R^{L} is optionally substituted 5-membered heteroaryl having 1-4 heteroatoms. In some embodiments, R^{L} is optionally substituted tetrazolyl. In some embodiments, R^{L} is tetrazolyl.

In some embodiments, a provided compound is a compound of the formula V below or a salt thereof:

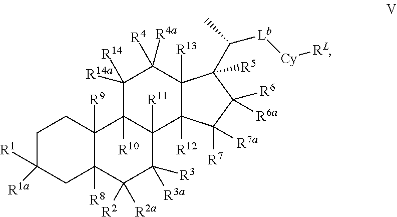

wherein:
   L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—; and
   each variable is independently as described herein.

In some embodiments, L^{b} is -L^{b}′-C(R′)₂—, wherein L^{b}′ is a covalent bond or an optionally substituted, bivalent C₁₋₉ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(S)N(R′)—, —C(NR′)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{b} is -L^{b}′-CHR′—. In some embodiments, L^{b} is -L^{b}′-C(R₃)₂—. In some embodiments, L^{b} is -L^{b}′-CHR₃—. In some embodiments, L^{b} is —(CH₂)m-.

In some embodiments, -Cy- is —Ar—, wherein —Ar— is an optionally substituted bivalent aryl (e.g., 6-14 membered) or heteroaryl (e.g., 5-14 membered having 1-10 heteroatoms) ring. In some embodiments, —Ar— is an optionally substituted bivalent heteroaryl (e.g., 5-14 membered having 1-10 heteroatoms) ring. In some embodiments, —Ar— is an optionally substituted bivalent 5-membered heteroaryl ring having 1-4 heteroatoms. In some embodiments, —Ar— is an optionally substituted bivalent 5-membered heteroaryl ring having 3 heteroatoms. In some embodiments, —Ar— is an optionally substituted bivalent 5-membered heteroaryl ring having 3 heteroatoms each independently selected from oxygen, nitrogen and sulfur. In some embodiments, —Ar— is an optionally substituted bivalent 5-membered heteroaryl ring having two nitrogen atoms and one oxygen or sulfur atom. In some embodiments, —Ar— is an optionally substituted bivalent 5-membered heteroaryl ring having three nitrogen atoms.

In some embodiments, a provided compound is a compound of the formula V-a below or a salt thereof:

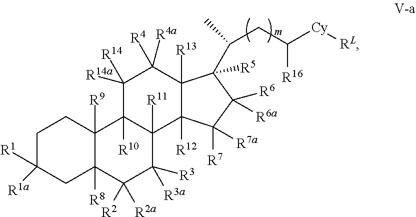

wherein each variable is independently as described herein.

In some embodiments, a provided compound is a compound of the formula V-b below or a salt thereof:

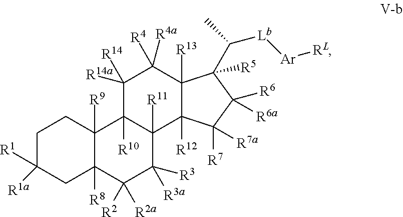

wherein each variable is independently as described herein.

In some embodiments, a provided compound is a compound of formula V-c or a salt thereof:

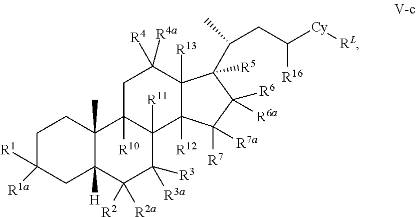

wherein each variable is independently as described herein.

In some embodiments, R¹⁶ is R₃ as described herein.

In some embodiments, R^{L} is —NR₉R₁₀, —N(R₁₁)C(O)R₁₂, —N(R₁₁)C(O)OR₁₂, —N(R₁₁)C(O)NR₉R₁₀, —N(R₁₁)S(O)₂NR₉R₁₀, —N(R₁₁)S(O)₂R₁₂, —N(R₁₁)C(O)N(R)S(O)₂R₁₂, or —N(R₁)C(O)N(R)S(O)₂NR₉R₁₀ as described herein.

In some embodiments, -Cy- is an optionally substituted bivalent 5-membered heteroaryl ring having three heteroatoms independently selected from nitrogen, oxygen and sulfur; preferably Ar has one oxygen or sulfur and two nitrogen ring heteroatoms or Ar has three nitrogen ring heteroatoms; or -Cy-R^{L} is a group selected from

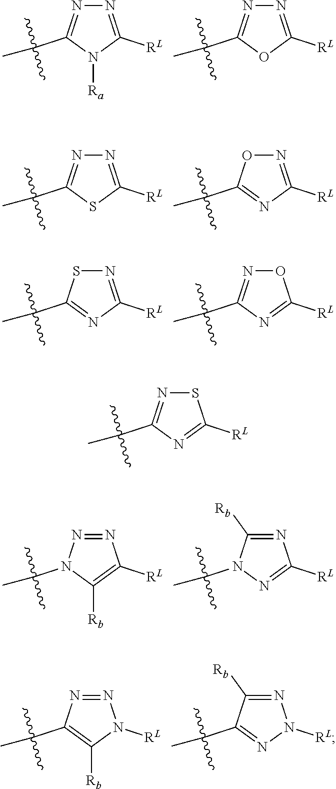

R^{a} is —H or an optionally substituted group selected from C₁₋₈ alkyl, C₂₋₈ alkenyl, C₂₋₈ alkynyl, C₃₋₈ cycloalkyl, —C(O)R₁₂, —C(O)OR₁₂, —C(O)NR₉R₁₀, —S(O)₂R₁₂, —C(O)N(R₉)S(O)₂R₁₂;
R_{b} is —H, halogen, —CF₃, —CN, —C(O)R₁₂, —C(O)OR₁₂, —C(O)NR₉R₁₀, —S(O)₂R₁₂, —C(O)N(R₉)S(O)₂R₁₂, or an optionally substituted group selected from C₁₋₈ alkyl, C₂₋₈ alkenyl, C₂₋₈ alkynyl, C₃₋₈ cycloalkyl, C₃₋₈ heterocycloalkyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl);
R¹⁶ is —H or an optionally substituted group selected from C₁₋₈ alkyl, C₂₋₈ alkenyl, C₂₋₈ alkynyl, aryl (e.g., C₆₋₁₄ aryl) and arylalkyl (e.g., C₆₋₁₄arylC₁₋₁₂alkyl); preferably R¹⁶ is —H or —CH₃;
R^{L} is —H, —NR₉R₁₀, —N(R₁₁)C(O)R₁₂, —N(R₁)C(O)OR₁₂, —N(R₁₁)C(O)NR₉R₁₀, —N(R₁₁)S(O)₂NR₉R₁₀, —N(R₁₁)S(O)₂R₁₂, —N(R₁₁)C(O)N(R₉)S(O)₂R₁₂, or —N(R₁)C(O)N(R₉)S(O)₂NR₉R₁₀, or an optionally substituted group selected from C₁₋₈ alkyl, C₂₋₈ alkenyl, C₂₋₈ alkynyl, C₃₋₈ cycloalkyl, C₃₋₈ heterocyclyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl); and
each of R₉, R₁₀, R₁₁, and R₁₂ is independently —H, or an optionally substituted group selected from C₁₋₈ alkyl, C₂₋₈ alkenyl, C₂₋₈ alkynyl, C₃₋₈ cycloalkyl, C₃₋₈ heterocycloalkyl (e.g., having 1-5 heteroatoms), aryl (e.g., C₆₋₁₄ aryl), heteroaryl (e.g., 5-14 membered heteroaryl having 1-10 heteroatoms), and benzyl; or R₉ and R₁₀, and/or R₉ and R₁₁, are taken together with their intervening atoms to form an optionally substituted 3-15 membered, monocyclic, bicyclic or polycyclic heterocyclyl ring having 1-5 heteroatoms (e.g., an optionally substituted heteroaryl (e.g., 5-14 membered heteroaryl having 1-10 heteroatoms)).

In some embodiments, R^{L} is —H. In some embodiments, R^{L} is —N(R′)₂. In some embodiments, R^{L} is —NR₉R₁₀. In some embodiments, R^{L} is —N(R′)C(O)R′. In some embodiments, R^{L} is —N(R₁₁)C(O)R₁₂. In some embodiments, R^{L} is —N(R′)C(O)OR′. In some embodiments, R^{L} is —N(R₁₁)C(O)OR₁₂. In some embodiments, R^{L} is —N(R′)C(O)N(R′)₂. In some embodiments, R^{L} is —N(R₁₁)C(O)NR₉R₁₀. In some embodiments, R^{L} is —N(R′)S(O)₂N(R′)₂. In some embodiments, R^{L} is —N(R₁₁)S(O)₂NR₉R₁₀. In some embodiments, R^{L} is —N(R′)S(O)₂R′. In some embodiments, R^{L} is —N(R₁₁)S(O)₂R₁₂. In some embodiments, R^{L} is —N(R′)C(O)N(R′)S(O)₂R′. In some embodiments, R^{L} is —N(R₁₁)C(O)N(R₉)S(O)₂R₁₂. In some embodiments, R^{L} is —N(R′)C(O)N(R′)S(O)₂N(R′). In some embodiments, R^{L} is —N(R₁₁)C(O)N(R₉)S(O)₂NR₉R₁₀. In some embodiments, R^{L} is an optionally substituted group selected from C₁₋₈ alkyl, C₂₋₈ alkenyl, C₂₋₈ alkynyl, C₃₋₈ cycloalkyl, C₃₋₈ heterocyclyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl).

In some embodiments, -Cy-R^{L}— is

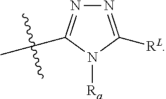

In some embodiments, -Cy-R^{L}— is

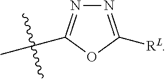

In some embodiments, -Cy-R^{L}— is

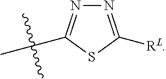

In some embodiments, -Cy-R^{L}— is

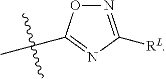

In some embodiments, -Cy-R^{L}— is

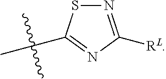

In some embodiments, -Cy-R^{L}— is

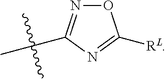

In some embodiments, -Cy-R^{L}— is

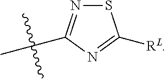

In some embodiments, -Cy-R^{L}— is

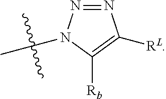

In some embodiments -Cy-R^{L}— is

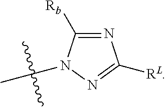

In some embodiments, -Cy-R^{L}— is

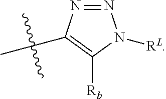

In some embodiments, -Cy-R^{L}— is

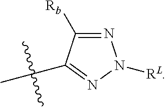

In some embodiments, a compound has a structure selected below or a salt thereof:

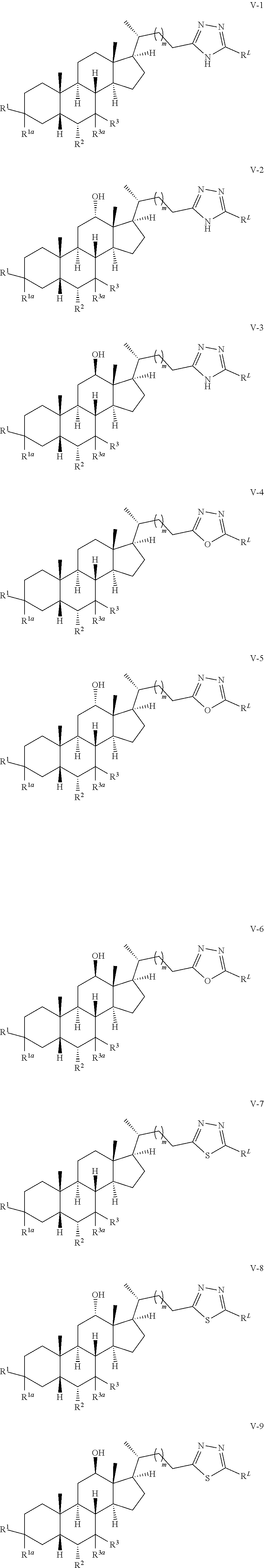

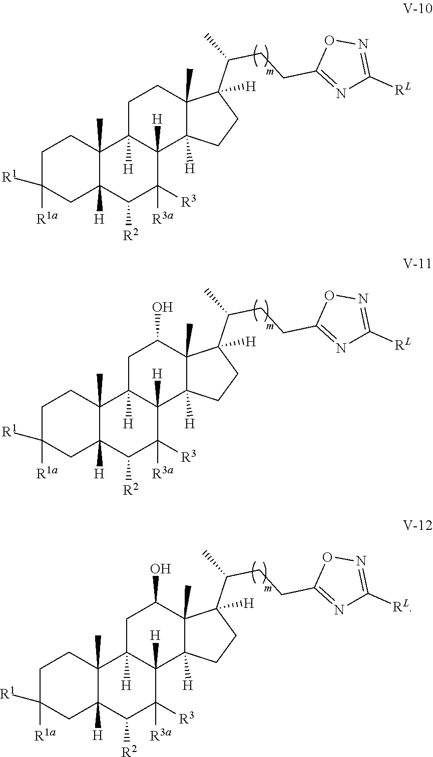

In some embodiments, a provided compound is a compound of formula J-I or a salt thereof:

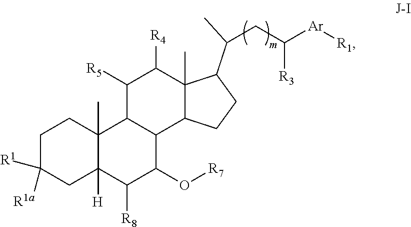

wherein:
   R₁ is —H, or an optionally substituted group selected from C₁₋₈ alkyl, C₂₋₈ alkenyl, C₂₋₈ alkynyl, C₃₋₈ cycloalkyl, C₃₋₈ heterocyclyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl), or R₁ is —NR₉R₁₀, —N(R₁₁)C(O)R₁₂, —N(R₁₁)C(O)OR₁₂, —N(R₁₁)C(O)NR₉R₁₀, —N(R₁₁)S(O)₂NR₉R₁₀, —N(R₁₁)S(O)₂R₁₂, —N(R₁₁)C(O)N(R₉)S(O)₂R₁₂, or —N(R₁₁)C(O)N(R₉)S(O)₂NR₉R₁₀;
Ar is an optionally substituted bivalent 5-membered heteroaryl ring having three heteroatoms independently selected from nitrogen, oxygen and sulfur; preferably Ar has one oxygen or sulfur and two nitrogen ring heteroatoms or Ar has three nitrogen ring heteroatoms; or —Ar—R₁ is a group selected from

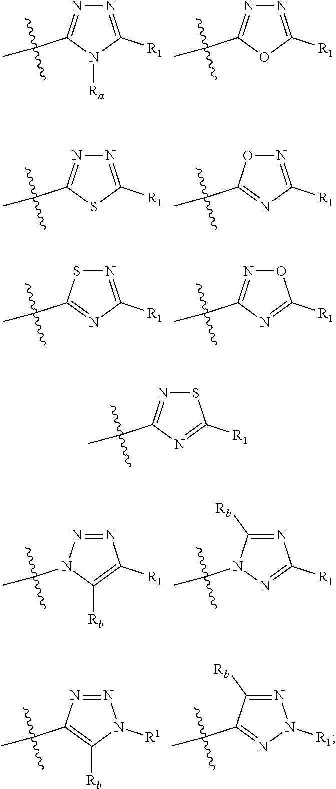

R^{a} is —H or an optionally substituted group selected from C₁₋₈ alkyl, C₂₋₈ alkenyl, C₂₋₈ alkynyl, C₃₋₈ cycloalkyl, —C(O)R₁₂, —C(O)OR₁₂, —C(O)NR₉R₁₀, —S(O)₂R₁₂, —C(O)N(R₉)S(O)₂R₁₂;
R_{b} is —H, halogen, —CF₃, —CN, —C(O)R₁₂, —C(O)OR₁₂, —C(O)NR₉R₁₀, —S(O)₂R₁₂, —C(O)N(R₉)S(O)₂R₁₂, or an optionally substituted group selected from C₁₋₈ alkyl, C₂₋₈ alkenyl, C₂₋₈ alkynyl, C₃₋₈ cycloalkyl, C₃₋₈ heterocycloalkyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl);
R₃ is —H or an optionally substituted group selected from C₁₋₈ alkyl, C₂₋₈ alkenyl, C₂₋₈ alkynyl, aryl (e.g., C₆₋₁₄ aryl) and arylalkyl (e.g., C₆₋₁₄arylC₁₋₁₂alkyl); preferably R¹⁶ is —H or —CH₃;
m is 0, 1, 2, or 3; preferably m is 0, 1, or 2;
R₄ is —H, —OH, —OSO₃H, —OAc or —OPO₃H₂; preferably R₄ is —H or —OH;
R₅ is —H, halogen, —CN, —N₃, —OH, —OSO₃H, —OAc, —OPO₃H₂, —SR₃, or —NHR₃; preferably R₅ is —H; or R₄ and R₅ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R₇ is —H or a hydroxyl protecting group (e.g., acetyl, trimethylsilyl, or benzyl); preferably R₇ is —H;
R₈ is —H, halogen, or an optionally substituted group selected from C₁₋₈ alkyl, C₂₋₈ alkenyl, C₂₋₈ alkynyl, and C₃₋₈ cycloalkyl; preferably R₈ is C₁₋₄ alkyl; more preferably Rₛ is ethyl.
each of R₉, R₁₀, R₁₁, and R₁₂ is independently —H, or an optionally substituted group selected from C₁₋₈ alkyl, C₂₋₈ alkenyl, C₂₋₈ alkynyl, C₃₋₈ cycloalkyl, C₃₋₈ heterocycloalkyl (e.g., having 1-5 heteroatoms), aryl (e.g., C₆₋₁₄ aryl), heteroaryl (e.g., 5-14 membered heteroaryl having 1-10 heteroatoms), and benzyl; or R₉ and R₁₀, and/or R₉ and R₁₁, are taken together with their intervening atoms to form an optionally substituted 3-15 membered, monocyclic, bicyclic or polycyclic heterocyclyl ring having 1-5 heteroatoms (e.g., an optionally substituted heteroaryl (e.g., 5-14 membered heteroaryl having 1-10 heteroatoms)); and
each other variable is independently as described herein.

In some embodiments, R₁ is —H, or an optionally substituted group selected from C₁₋₈ alkyl, C₂₋₈ alkenyl, C₂₋₈ alkynyl, C₃₋₈ cycloalkyl, C₃₋₈ heterocyclyl (e.g., having 1-5 heteroatoms), aryl (e.g., C₆₋₁₄ aryl), heteroaryl (e.g., 5-14 membered heteroaryl having 1-10 heteroatoms), and arylalkyl (e.g., C₆₋₁₄arylC₁₋₁₂alkyl), or R₁ is —NR₉R₁₀, —N(R₁₁)C(O)R₁₂, —N(R₁₁)C(O)OR₁₂, —N(R₁₁)C(O)NR₉R₁₀, —N(R₁₁)S(O)₂NR₉R₁₀, —N(R₁₁)S(O)₂R₁₂, —N(R₁₁)C(O)N(R₉)S(O)₂R₁₂, or —N(R₁)C(O)N(R₉)S(O)₂NR₉R₁₀.

In some embodiments, —Ar—R₁ is

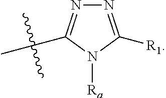

In some embodiments, —Ar—R₁ is

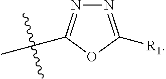

In some embodiments, —Ar—R₁ is

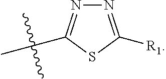

In some embodiments, —Ar—R₁ is

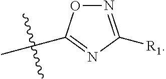

In some embodiments, —Ar—R₁ is

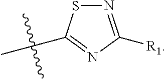

In some embodiments, —Ar—R₁ is

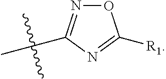

In some embodiments, —Ar—R₁ is

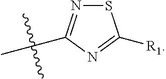

In some embodiments, —Ar—R₁ is

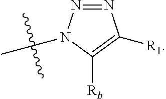

In some embodiments, —Ar—R₁ is

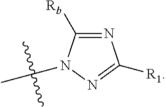

In some embodiments, —Ar-Ri is

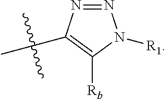

In some embodiments, —Ar—R₁ is

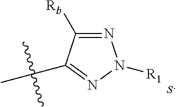

In some embodiments, a provided compound is a compound of formula J-I′ or a salt thereof:

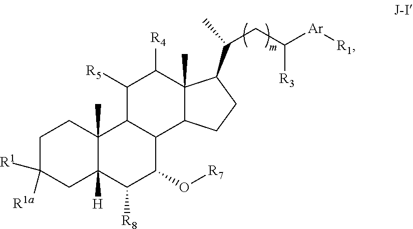

wherein variable is independently as described herein.

In some embodiments, a provided compound is a compound of formula J-IA or a salt thereof:

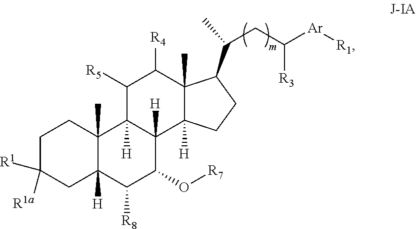

wherein variable is independently as described herein.

In some embodiments, R₃, R₄, R₅, and R₇ are each —H, and R₈ is ethyl. In some embodiments, R₃, R₄, R₅, and R₇ are each —H, R₈ is ethyl, and each of R¹ and R^{1a} is independently —H or halogen. In some embodiments, R₃, R₄, R₅, and R₇ are each —H, R₈ is ethyl, and each of R¹ and R^{1a} is independently —H or —F. In some embodiments, R₃, R₄, R₅, and R₇ are each —H, R₈ is ethyl, and each of R¹ and R^{1a} is independently —H. In some embodiments, R₃, R₄, R₅, and R₇ are each —H, R₈ is ethyl, and each of R¹ and R^{1a} is independently —F. In some embodiments, R₃, R₄, R₅, and R₇ are each —H, R₈ is ethyl, and one of R¹ and R^{1a} is —H and the other is —F.

In some embodiments, a provided compound is a compound of formula J-II or a salt thereof:

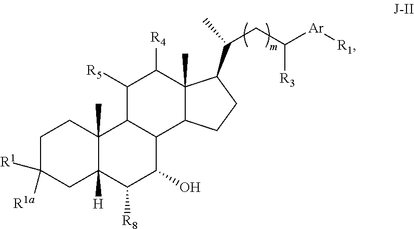

wherein variable is independently as described herein.

In some embodiments, a provided compound is a compound of formula J-III or a salt thereof: R

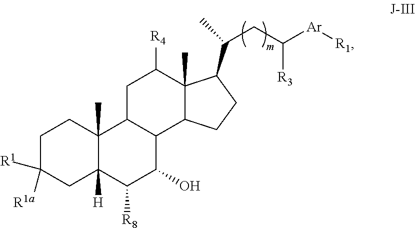

wherein variable is independently as described herein.

In some embodiments, a provided compound is a compound of a formula selected from below or a salt thereof:

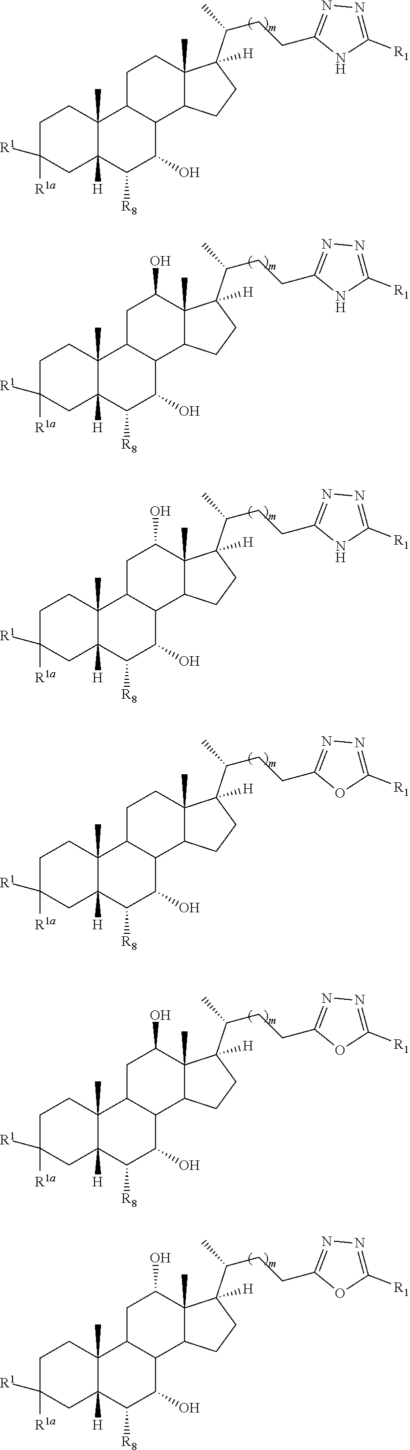

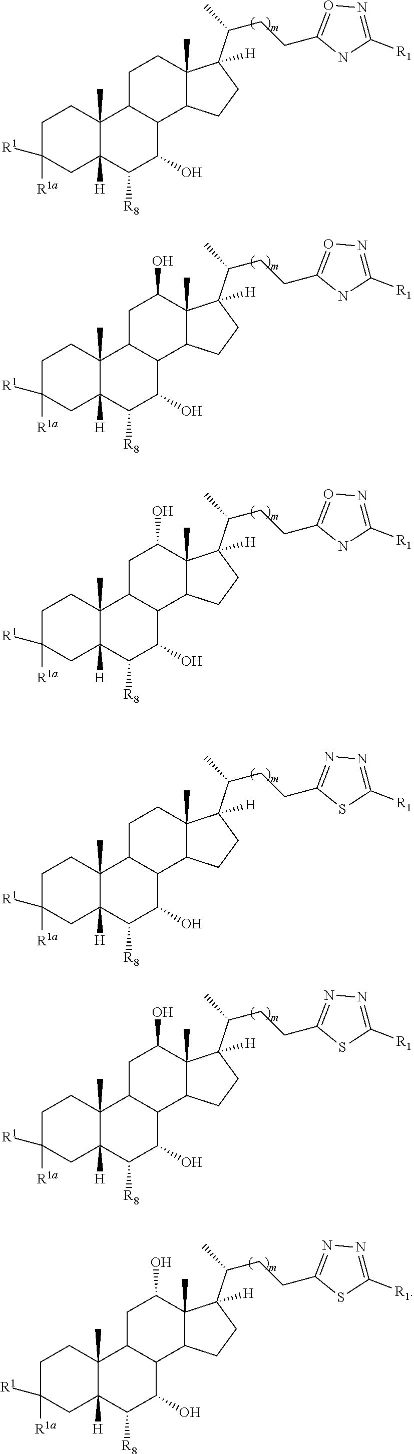

In some embodiments, a provided compound is a compound of formula J-IV or a salt thereof:

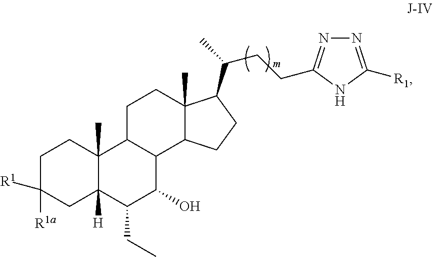

wherein variable is independently as described herein.

In some embodiments, a provided compound is a compound of formula J-V or a salt thereof:

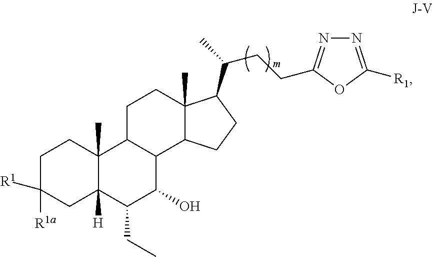

wherein variable is independently as described herein.

In some embodiments, a provided compound is a compound of formula J-VI or a salt thereof:

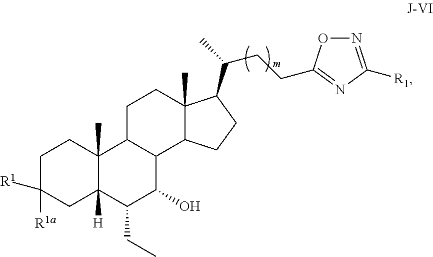

wherein variable is independently as described herein.

In some embodiments, a provided compound is a compound of formula J-VII or a salt thereof:

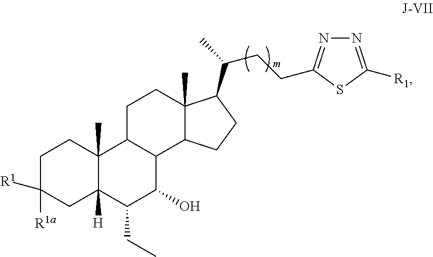

wherein variable is independently as described herein.

In some embodiments, a provided compound is a compound of formula J-VIII or a salt thereof:

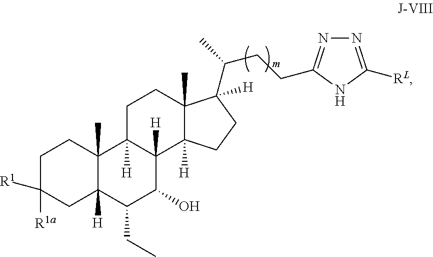

wherein variable is independently as described herein.

In some embodiments, a provided compound is a compound of formula J-VIII-1 or a salt thereof:

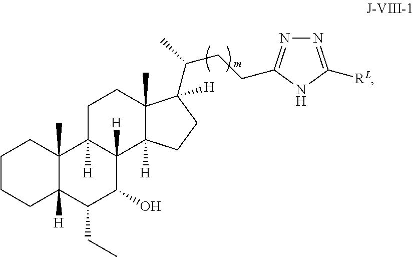

wherein variable is independently as described herein.

In some embodiments, m and R₁, or m and R^{L}, are as described in any one of Table 1 to Table 7. In some embodiments, they are as described in Table 1. In some embodiments, they are as described in Table 2. In some embodiments, they are as described in Table 3. In some embodiments, they are as described in Table 4. In some embodiments, they are as described in Table 5. In some embodiments, they are as described in Table 7. In some embodiments, they are as described in Table 7.

In some embodiments, a compound has the structure of formula J-V wherein its m and R^{L} is described in Table 7 (compounds J-V-1 to J-V-I-351, wherein J-V-zz has its m and R^{L} of entry zz (e.g., compound J-V-1 has its m and R₁ of entry 1)) or a salt thereof, and wherein R¹ and R^{1a} are —H. In some embodiments, a compound has the structure of formula J-VI wherein its m and R^{L} is described in Table 7 (compounds J-VI-1 to J-VI-351, wherein J-VI-zz has its m and R^{L} of entry zz (e.g., compound J-VI-1 has its m and R₁ of entry 1)) or a salt thereof, and wherein R¹ and R^{1a} are —H. In some embodiments, a compound has the structure of formula J-VII wherein its m and R^{L} is described in Table 7 (compounds I-VII-1 to J-VII-351, wherein J-VI-zz has its m and R^{L} of entry zz (e.g., compound J-VII-I-1 has its m and R₁ of entry 1)) or a salt thereof, and wherein R¹ and R^{1a} are —H. In some embodiments, a compound has the structure of formula J-VIII-I wherein its m and R^{L} is described in Table 7 (compounds J-VIII-I-1 to J-VIII-I-351, wherein J-VIII-I-zz has its m and R^{L} of entry zz (e.g., compound J-VIII-I-1 has its m and R₁ of entry 1)) or a salt thereof.

TABLE 7
Certain examples of m and R^{L}/R₁.

| Entry | m | R^{L}/R₁ |
|---|---|---|
| 1 | 0 | H |
| 2 | 0 | 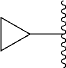 [SMILES: *C1CC1] |
| 3 | 0 | 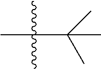 [SMILES: *C(C)(C)C] |
| 4 | 0 | Benzyl |
| 5 | 0 | 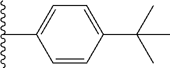 [SMILES: *c1ccc(C(C)(C)C)cc1] |
| 6 | 0 | NH₂ |
| 7 | 0 | NMe₂ |
| 8 | 0 | 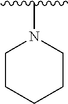 [SMILES: *N1CCCCC1] |
| 9 | 0 | 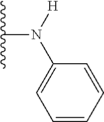 [SMILES: *Nc1ccccc1] |
| 10 | 0 | 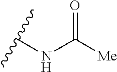 [SMILES: *NC(C)=O] |
| 11 | 0 | 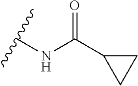 [SMILES: *NC(=O)C1CC1] |
| 12 | 0 | 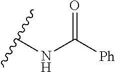 [SMILES: *NC(=O)c1ccccc1] |
| 13 | 0 | 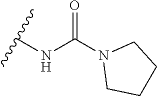 [SMILES: *NC(=O)N1CCCC1] |
| 14 | 0 | 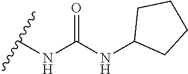 [SMILES: *NC(=O)NC1CCCC1] |
| 15 | 0 | 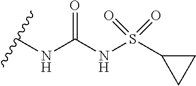 [SMILES: *NC(=O)NS(=O)(=O)C1CC1] |
| 16 | 0 | 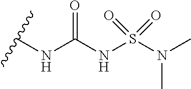 [SMILES: *NC(=O)NS(=O)(=O)N(C)C] |
| 17 | 0 | 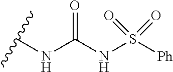 [SMILES: *NC(=O)NS(=O)(=O)c1ccccc1] |
| 18 | 0 | 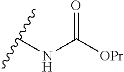 |
| 19 | 0 | 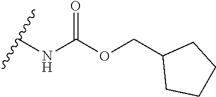 [SMILES: *NC(=O)OCC1CCCC1] |
| 20 | 0 | 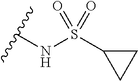 [SMILES: *NS(=O)(=O)C1CC1] |
| 21 | 0 | 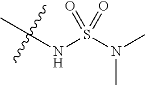 [SMILES: *NS(=O)(=O)N(C)C] |
| 22 | 0 | 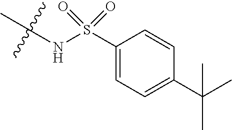 [SMILES: *NS(=O)(=O)c1ccc(C(C)(C)C)cc1] |
| 23 | 0 | 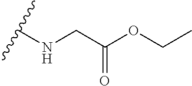 [SMILES: *NCC(=O)OCC] |
| 24 | 0 | 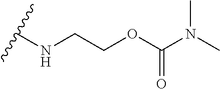 [SMILES: *NCCOC(=O)N(C)C] |
| 25 | 0 | 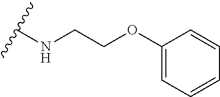 [SMILES: *NCCOc1ccccc1] |
| 26 | 0 | 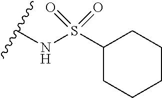 [SMILES: *NS(=O)(=O)C1CCCCC1] |
| 27 | 0 | 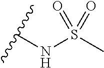 [SMILES: *NS(C)(=O)=O] |
| 28 | 0 | 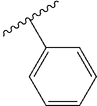 [SMILES: *c1ccccc1] |
| 29 | 0 | 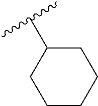 [SMILES: *C1CCCCC1] |
| 30 | 1 | H |
| 31 | 1 | 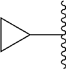 [SMILES: *C1CC1] |
| 32 | 1 | 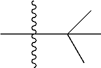 [SMILES: *C(C)(C)C] |
| 33 | 1 | Benzyl |
| 34 | 1 | 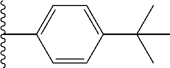 [SMILES: *c1ccc(C(C)(C)C)cc1] |
| 35 | 1 | NH₂ |
| 36 | 1 | NMe₂ |
| 37 | 1 | 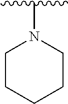 [SMILES: *N1CCCCC1] |
| 38 | 1 | 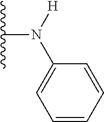 [SMILES: *Nc1ccccc1] |
| 39 | 1 | 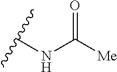 [SMILES: *NC(C)=O] |
| 40 | 1 | 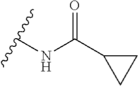 [SMILES: *NC(=O)C1CC1] |
| 41 | 1 | 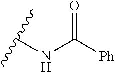 [SMILES: *NC(=O)c1ccccc1] |
| 42 | 1 | 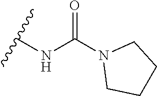 [SMILES: *NC(=O)N1CCCC1] |
| 43 | 1 | 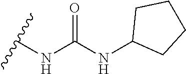 [SMILES: *NC(=O)NC1CCCC1] |
| 44 | 1 | 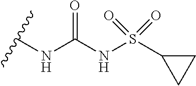 [SMILES: *NC(=O)NS(=O)(=O)C1CC1] |
| 45 | 1 | 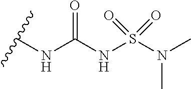 [SMILES: *NC(=O)NS(=O)(=O)N(C)C] |
| 46 | 1 | 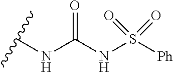 [SMILES: *NC(=O)NS(=O)(=O)c1ccccc1] |
| 47 | 1 | 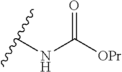 |
| 48 | 1 | 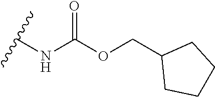 [SMILES: *NC(=O)OCC1CCCC1] |
| 49 | 1 | 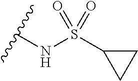 [SMILES: *NS(=O)(=O)C1CC1] |
| 50 | 1 | 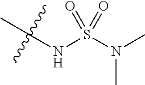 [SMILES: *NS(=O)(=O)N(C)C] |
| 51 | 1 | 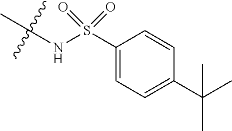 [SMILES: *NS(=O)(=O)c1ccc(C(C)(C)C)cc1] |
| 52 | 1 | 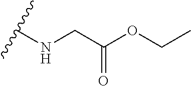 [SMILES: *NCC(=O)OCC] |
| 53 | 1 | 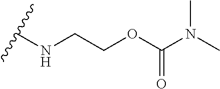 [SMILES: *NCCOC(=O)N(C)C] |
| 54 | 1 | 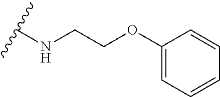 [SMILES: *NCCOc1ccccc1] |
| 55 | 1 | 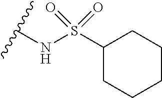 [SMILES: *NS(=O)(=O)C1CCCCC1] |
| 56 | 1 | 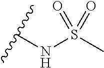 [SMILES: *NS(C)(=O)=O] |
| 57 | 1 | 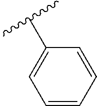 [SMILES: *c1ccccc1] |
| 58 | 1 | 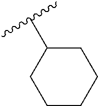 [SMILES: *C1CCCCC1] |
| 59 | 2 | H |
| 60 | 2 | 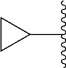 [SMILES: *C1CC1] |
| 61 | 2 | 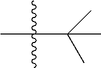 [SMILES: *C(C)(C)C] |
| 62 | 2 | Benzyl |
| 63 | 2 | 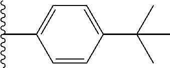 [SMILES: *c1ccc(C(C)(C)C)cc1] |
| 64 | 2 | NH₂ |
| 65 | 2 | NMe₂ |
| 66 | 2 | 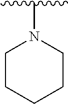 [SMILES: *N1CCCCC1] |
| 67 | 2 | 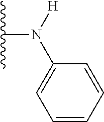 [SMILES: *Nc1ccccc1] |
| 68 | 2 | 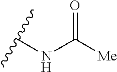 [SMILES: *NC(C)=O] |
| 69 | 2 | 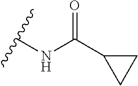 [SMILES: *NC(=O)C1CC1] |
| 70 | 2 | 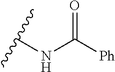 [SMILES: *NC(=O)c1ccccc1] |
| 71 | 2 | 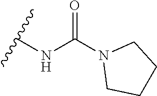 [SMILES: *NC(=O)N1CCCC1] |
| 72 | 2 | 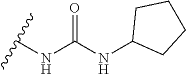 [SMILES: *NC(=O)NC1CCCC1] |
| 73 | 2 | 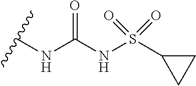 [SMILES: *NC(=O)NS(=O)(=O)C1CC1] |
| 74 | 2 | 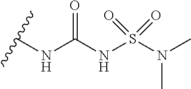 [SMILES: *NC(=O)NS(=O)(=O)N(C)C] |
| 75 | 2 | 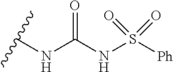 [SMILES: *NC(=O)NS(=O)(=O)c1ccccc1] |
| 76 | 2 | 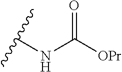 |
| 77 | 2 | 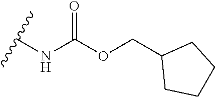 [SMILES: *NC(=O)OCC1CCCC1] |
| 78 | 2 | 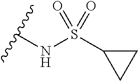 [SMILES: *NS(=O)(=O)C1CC1] |
| 79 | 2 | 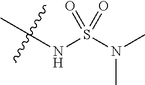 [SMILES: *NS(=O)(=O)N(C)C] |
| 80 | 2 | 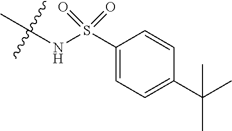 [SMILES: *NS(=O)(=O)c1ccc(C(C)(C)C)cc1] |
| 81 | 2 | 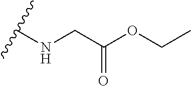 [SMILES: *NCC(=O)OCC] |
| 82 | 2 | 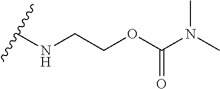 [SMILES: *NCCOC(=O)N(C)C] |
| 83 | 2 | 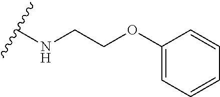 [SMILES: *NCCOc1ccccc1] |
| 84 | 2 | 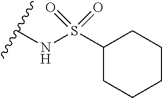 [SMILES: *NS(=O)(=O)C1CCCCC1] |
| 85 | 2 | 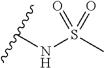 [SMILES: *NS(C)(=O)=O] |
| 86 | 2 | 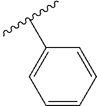 [SMILES: *c1ccccc1] |
| 87 | 2 | 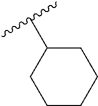 [SMILES: *C1CCCCC1] |
| 88 | 0 | 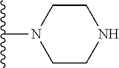 [SMILES: *N1CCNCC1] |
| 89 | 0 | 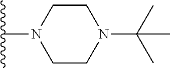 [SMILES: *N1CCN(C(C)(C)C)CC1] |
| 90 | 0 | 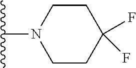 [SMILES: *N1CCC(F)(F)CC1] |
| 91 | 0 | 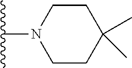 [SMILES: *N1CCC(C)(C)CC1] |
| 92 | 0 | 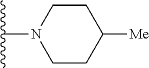 [SMILES: *N1CCC(C)CC1] |
| 93 | 0 | 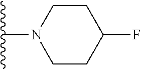 [SMILES: *N1CCC(F)CC1] |
| 94 | 0 | 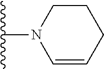 [SMILES: *N1C=CCCC1] |
| 95 | 0 | 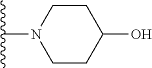 [SMILES: *N1CCC(O)CC1] |
| 96 | 0 | 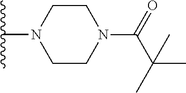 [SMILES: *N1CCN(C(=O)C(C)(C)C)CC1] |
| 97 | 0 | 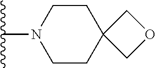 [SMILES: *N1CCC2(CC1)COC2] |
| 98 | 0 | 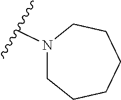 [SMILES: *N1CCCCCC1] |
| 99 | 0 | 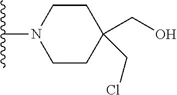 [SMILES: *N1CCC(CO)(CCl)CC1] |
| 100 | 0 | 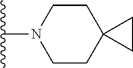 [SMILES: *N1CCC2(CC1)CC2] |
| 101 | 0 | 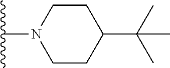 [SMILES: *N1CCC(C(C)(C)C)CC1] |
| 102 | 0 | 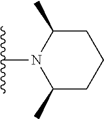 [SMILES: *N1[C@H](C)CCC[C@@H]1C] |
| 103 | 0 | 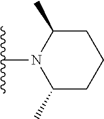 [SMILES: *N1[C@@H](C)CCC[C@@H]1C] |
| 104 | 0 | 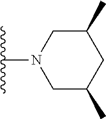 [SMILES: *N1C[C@H](C)C[C@H](C)C1] |
| 105 | 0 | 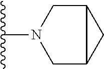 [SMILES: *N1CC2CC2C1] |
| 106 | 0 | 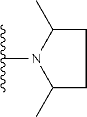 [SMILES: *N1C(C)CCC1C] |
| 107 | 0 | 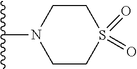 [SMILES: *N1CCS(=O)(=O)CC1] |
| 108 | 0 | 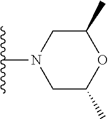 [SMILES: *N1C[C@@H](C)O[C@H](C)C1] |
| 109 | 0 | 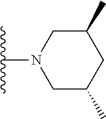 [SMILES: *N1C[C@@H](C)C[C@H](C)C1] |
| 110 | 0 | 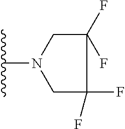 [SMILES: *N1CC(F)(F)C(F)(F)C1] |
| 111 | 0 | 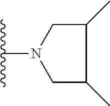 [SMILES: *N1CC(C)C(C)C1] |
| 112 | 0 | 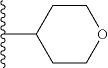 [SMILES: *C1CCOCC1] |
| 113 | 0 | 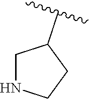 [SMILES: *C1CCNC1] |
| 114 | 0 | 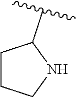 [SMILES: *C1CCCN1] |
| 115 | 0 | 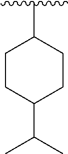 [SMILES: *C1CCC(C(C)C)CC1] |
| 116 | 0 | 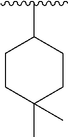 [SMILES: *C1CCC(C)(C)CC1] |
| 117 | 0 | 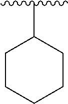 [SMILES: *C1CCCCC1] |
| 118 | 0 | 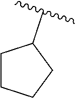 [SMILES: *C1CCCC1] |
| 119 | 0 | 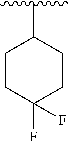 [SMILES: *C1CCC(F)(F)CC1] |
| 120 | 0 | 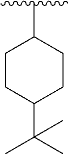 [SMILES: *C1CCC(C(C)(C)C)CC1] |
| 121 | 0 | 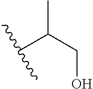 [SMILES: *C(C)CO] |
| 122 | 0 | 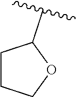 [SMILES: *C1CCCO1] |
| 123 | 0 | 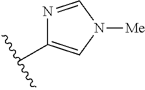 [SMILES: *c1cn(C)cn1] |
| 124 | 0 | 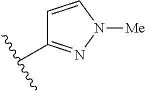 [SMILES: *c1ccn(C)n1] |
| 125 | 0 | 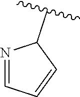 [SMILES: *C1C=CC=N1] |
| 126 | 0 | 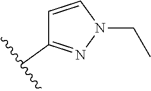 [SMILES: *c1ccn(CC)n1] |
| 127 | 0 | 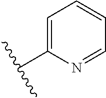 [SMILES: *c1ccccn1] |
| 128 | 0 | 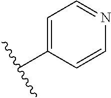 [SMILES: *c1ccncc1] |
| 129 | 0 | 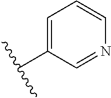 [SMILES: *c1cccnc1] |
| 130 | 0 | 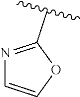 [SMILES: *c1ncco1] |
| 131 | 0 | 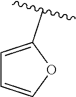 [SMILES: *c1ccco1] |
| 132 | 0 | 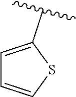 [SMILES: *c1cccs1] |
| 133 | 0 | 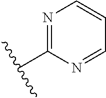 [SMILES: *c1ncccn1] |
| 134 | 0 | 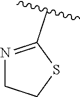 [SMILES: *C1=NCCS1] |
| 135 | 0 | 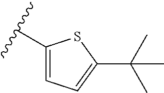 [SMILES: *c1ccc(C(C)(C)C)s1] |
| 136 | 0 | 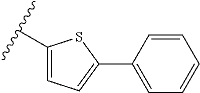 [SMILES: *c1ccc(-c2ccccc2)s1] |
| 137 | 0 | 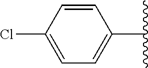 [SMILES: *c1ccc(Cl)cc1] |
| 138 | 0 | 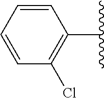 [SMILES: *c1ccccc1Cl] |
| 139 | 0 | 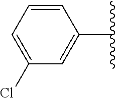 [SMILES: *c1cccc(Cl)c1] |
| 140 | 0 | 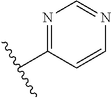 [SMILES: *c1ccncn1] |
| 141 | 0 | 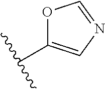 [SMILES: *c1cnco1] |
| 142 | 0 | 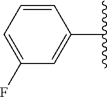 [SMILES: *c1cccc(F)c1] |
| 143 | 0 | 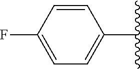 [SMILES: *c1ccc(F)cc1] |
| 144 | 0 | 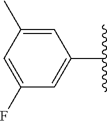 [SMILES: *c1cc(C)cc(F)c1] |
| 145 | 0 | 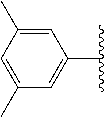 [SMILES: *c1cc(C)cc(C)c1] |
| 146 | 0 | 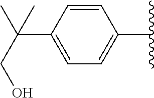 [SMILES: *c1ccc(C(C)(C)CO)cc1] |
| 147 | 0 | 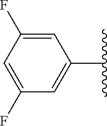 [SMILES: *c1cc(F)cc(F)c1] |
| 148 | 0 | 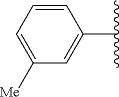 [SMILES: *c1cccc(C)c1] |
| 149 | 0 | 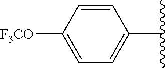 [SMILES: *c1ccc(OC(F)(F)F)cc1] |
| 150 | 0 | 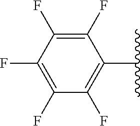 [SMILES: *c1c(F)c(F)c(F)c(F)c1F] |
| 151 | 0 | 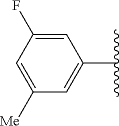 [SMILES: *c1cc(C)cc(F)c1] |
| 152 | 0 | 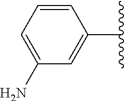 [SMILES: *c1cccc(N)c1] |
| 153 | 0 | 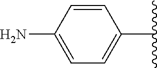 [SMILES: *c1ccc(N)cc1] |
| 154 | 0 | 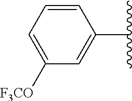 [SMILES: *c1cccc(OC(F)(F)F)c1] |
| 155 | 0 | 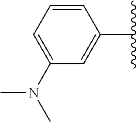 [SMILES: *c1cccc(N(C)C)c1] |
| 156 | 0 | 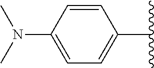 [SMILES: *c1ccc(N(C)C)cc1] |
| 157 | 0 | 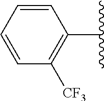 [SMILES: *c1ccccc1C(F)(F)F] |
| 158 | 0 | 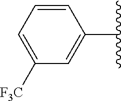 [SMILES: *c1cccc(C(F)(F)F)c1] |
| 159 | 0 | 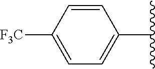 [SMILES: *c1ccc(C(F)(F)F)cc1] |
| 160 | 0 | 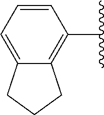 [SMILES: *c1cccc2c1CCC2] |
| 161 | 0 | 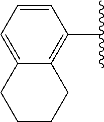 [SMILES: *c1cccc2c1CCCC2] |
| 162 | 0 | 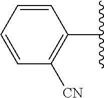 [SMILES: *c1ccccc1C#N] |
| 163 | 0 | 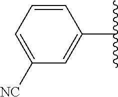 [SMILES: *c1cccc(C#N)c1] |
| 164 | 0 | 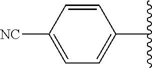 [SMILES: *c1ccc(C#N)cc1] |
| 165 | 0 | 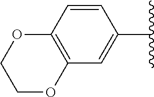 [SMILES: *c1ccc2c(c1)OCCO2] |
| 166 | 0 | 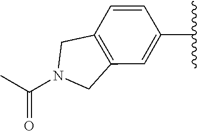 [SMILES: *c1ccc2c(c1)CN(C(C)=O)C2] |
| 167 | 0 | 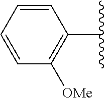 [SMILES: *c1ccccc1OC] |
| 168 | 0 | 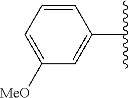 [SMILES: *c1cccc(OC)c1] |
| 169 | 0 | 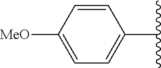 [SMILES: *c1ccc(OC)cc1] |
| 170 | 0 | 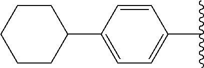 [SMILES: *c1ccc(C2CCCCC2)cc1] |
| 171 | 0 | 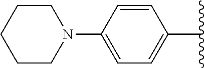 [SMILES: *c1ccc(N2CCCCC2)cc1] |
| 172 | 0 | 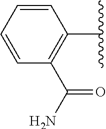 [SMILES: *c1ccccc1C(N)=O] |
| 173 | 0 | 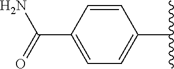 [SMILES: *c1ccc(C(N)=O)cc1] |
| 174 | 0 | 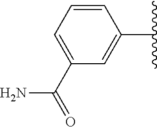 [SMILES: *c1cccc(C(N)=O)c1] |
| 175 | 0 | 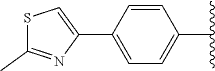 [SMILES: *c1ccc(-c2csc(C)n2)cc1] |
| 176 | 1 | 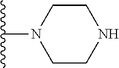 [SMILES: *N1CCNCC1] |
| 177 | 1 | 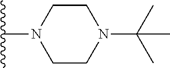 [SMILES: *N1CCN(C(C)(C)C)CC1] |
| 178 | 1 | 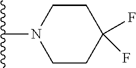 [SMILES: *N1CCC(F)(F)CC1] |
| 179 | 1 | 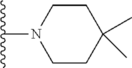 [SMILES: *N1CCC(C)(C)CC1] |
| 180 | 1 | 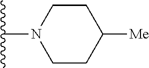 [SMILES: *N1CCC(C)CC1] |
| 181 | 1 | 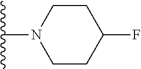 [SMILES: *N1CCC(F)CC1] |
| 182 | 1 | 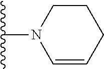 [SMILES: *N1C=CCCC1] |
| 183 | 1 | 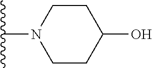 [SMILES: *N1CCC(O)CC1] |
| 184 | 1 | 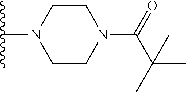 [SMILES: *N1CCN(C(=O)C(C)(C)C)CC1] |
| 185 | 1 | 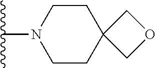 [SMILES: *N1CCC2(CC1)COC2] |
| 186 | 1 | 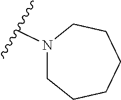 [SMILES: *N1CCCCCC1] |
| 187 | 1 | 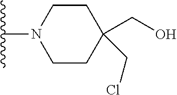 [SMILES: *N1CCC(CO)(CCl)CC1] |
| 188 | 1 | 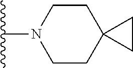 [SMILES: *N1CCC2(CC1)CC2] |
| 189 | 1 | 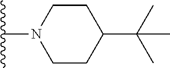 [SMILES: *N1CCC(C(C)(C)C)CC1] |
| 190 | 1 | 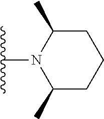 [SMILES: *N1[C@H](C)CCC[C@@H]1C] |
| 191 | 1 | 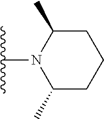 [SMILES: *N1[C@@H](C)CCC[C@@H]1C] |
| 192 | 1 | 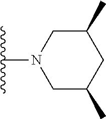 [SMILES: *N1C[C@H](C)C[C@H](C)C1] |
| 193 | 1 | 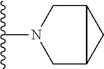 [SMILES: *N1CC2CC2C1] |
| 194 | 1 | 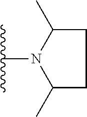 [SMILES: *N1C(C)CCC1C] |
| 195 | 1 | 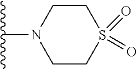 [SMILES: *N1CCS(=O)(=O)CC1] |
| 196 | 1 | 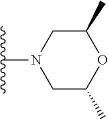 [SMILES: *N1C[C@@H](C)O[C@H](C)C1] |
| 197 | 1 | 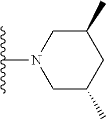 [SMILES: *N1C[C@@H](C)C[C@H](C)C1] |
| 198 | 1 | 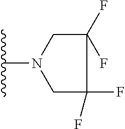 [SMILES: *N1CC(F)(F)C(F)(F)C1] |
| 199 | 1 | 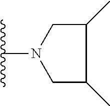 [SMILES: *N1CC(C)C(C)C1] |
| 200 | 1 | 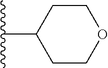 [SMILES: *C1CCOCC1] |
| 201 | 1 | 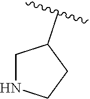 [SMILES: *C1CCNC1] |
| 202 | 1 | 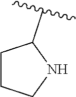 [SMILES: *C1CCCN1] |
| 203 | 1 | 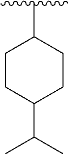 [SMILES: *C1CCC(C(C)C)CC1] |
| 204 | 1 | 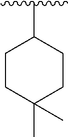 [SMILES: *C1CCC(C)(C)CC1] |
| 205 | 1 | 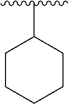 [SMILES: *C1CCCCC1] |
| 206 | 1 | 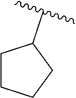 [SMILES: *C1CCCC1] |
| 207 | 1 | 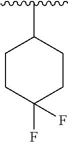 [SMILES: *C1CCC(F)(F)CC1] |
| 208 | 1 | 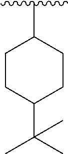 [SMILES: *C1CCC(C(C)(C)C)CC1] |
| 209 | 1 | 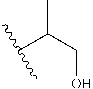 [SMILES: *C(C)CO] |
| 210 | 1 | 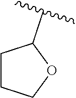 [SMILES: *C1CCCO1] |
| 211 | 1 | 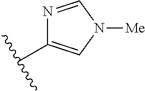 [SMILES: *c1cn(C)cn1] |
| 212 | 1 | 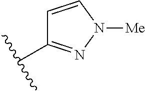 [SMILES: *c1ccn(C)n1] |
| 213 | 1 | 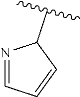 [SMILES: *C1C=CC=N1] |
| 214 | 1 | 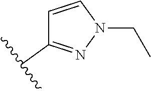 [SMILES: *c1ccn(CC)n1] |
| 215 | 1 | 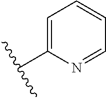 [SMILES: *c1ccccn1] |
| 216 | 1 | 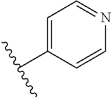 [SMILES: *c1ccncc1] |
| 217 | 1 | 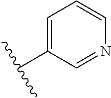 [SMILES: *c1cccnc1] |
| 218 | 1 | 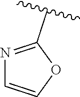 [SMILES: *c1ncco1] |
| 219 | 1 | 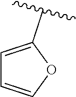 [SMILES: *c1ccco1] |
| 220 | 1 | 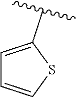 [SMILES: *c1cccs1] |
| 221 | 1 | 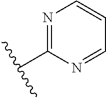 [SMILES: *c1ncccn1] |
| 222 | 1 | 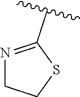 [SMILES: *C1=NCCS1] |
| 223 | 1 | 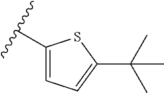 [SMILES: *c1ccc(C(C)(C)C)s1] |
| 224 | 1 | 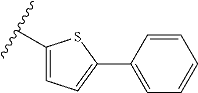 [SMILES: *c1ccc(-c2ccccc2)s1] |
| 225 | 1 | 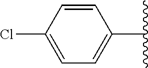 [SMILES: *c1ccc(Cl)cc1] |
| 226 | 1 | 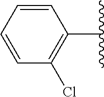 [SMILES: *c1ccccc1Cl] |
| 227 | 1 | 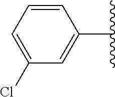 [SMILES: *c1cccc(Cl)c1] |
| 228 | 1 | 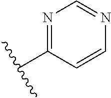 [SMILES: *c1ccncn1] |
| 229 | 1 | 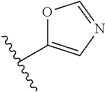 [SMILES: *c1cnco1] |
| 230 | 1 | 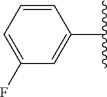 [SMILES: *c1cccc(F)c1] |
| 231 | 1 | 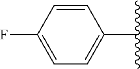 [SMILES: *c1ccc(F)cc1] |
| 232 | 1 | 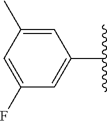 [SMILES: *c1cc(C)cc(F)c1] |
| 233 | 1 | 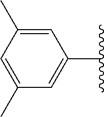 [SMILES: *c1cc(C)cc(C)c1] |
| 234 | 1 | 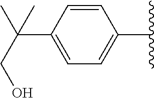 [SMILES: *c1ccc(C(C)(C)CO)cc1] |
| 235 | 1 | 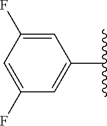 [SMILES: *c1cc(F)cc(F)c1] |
| 236 | 1 | 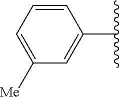 [SMILES: *c1cccc(C)c1] |
| 237 | 1 | 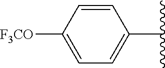 [SMILES: *c1ccc(OC(F)(F)F)cc1] |
| 238 | 1 | 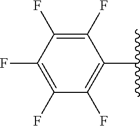 [SMILES: *c1c(F)c(F)c(F)c(F)c1F] |
| 239 | 1 | 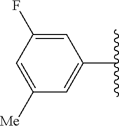 [SMILES: *c1cc(C)cc(F)c1] |
| 240 | 1 | 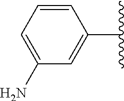 [SMILES: *c1cccc(N)c1] |
| 241 | 1 | 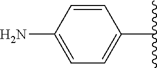 [SMILES: *c1ccc(N)cc1] |
| 242 | 1 | 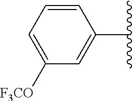 [SMILES: *c1cccc(OC(F)(F)F)c1] |
| 243 | 1 | 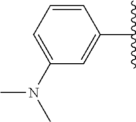 [SMILES: *c1cccc(N(C)C)c1] |
| 244 | 1 | 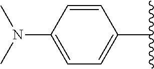 [SMILES: *c1ccc(N(C)C)cc1] |
| 245 | 1 | 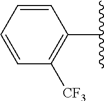 [SMILES: *c1ccccc1C(F)(F)F] |
| 246 | 1 | 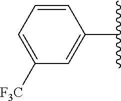 [SMILES: *c1cccc(C(F)(F)F)c1] |
| 247 | 1 | 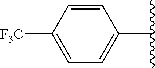 [SMILES: *c1ccc(C(F)(F)F)cc1] |
| 248 | 1 | 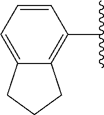 [SMILES: *c1cccc2c1CCC2] |
| 249 | 1 | 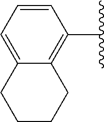 [SMILES: *c1cccc2c1CCCC2] |
| 250 | 1 | 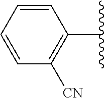 [SMILES: *c1ccccc1C#N] |
| 251 | 1 | 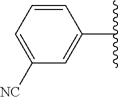 [SMILES: *c1cccc(C#N)c1] |
| 252 | 1 | 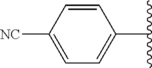 [SMILES: *c1ccc(C#N)cc1] |
| 253 | 1 | 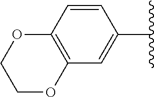 [SMILES: *c1ccc2c(c1)OCCO2] |
| 254 | 1 | 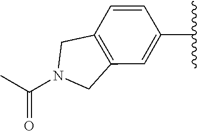 [SMILES: *c1ccc2c(c1)CN(C(C)=O)C2] |
| 255 | 1 | 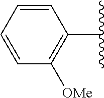 [SMILES: *c1ccccc1OC] |
| 256 | 1 | 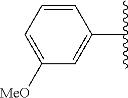 [SMILES: *c1cccc(OC)c1] |
| 257 | 1 | 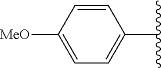 [SMILES: *c1ccc(OC)cc1] |
| 258 | 1 | 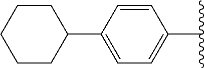 [SMILES: *c1ccc(C2CCCCC2)cc1] |
| 259 | 1 | 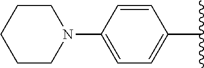 [SMILES: *c1ccc(N2CCCCC2)cc1] |
| 260 | 1 | 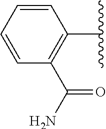 [SMILES: *c1ccccc1C(N)=O] |
| 261 | 1 | 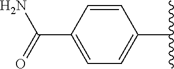 [SMILES: *c1ccc(C(N)=O)cc1] |
| 262 | 1 | 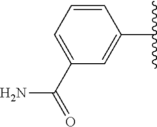 [SMILES: *c1cccc(C(N)=O)c1] |
| 263 | 1 | 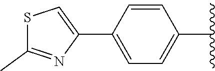 [SMILES: *c1ccc(-c2csc(C)n2)cc1] |
| 264 | 2 | 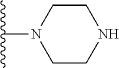 [SMILES: *N1CCNCC1] |
| 265 | 2 | 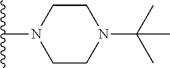 [SMILES: *N1CCN(C(C)(C)C)CC1] |
| 266 | 2 | 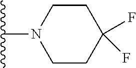 [SMILES: *N1CCC(F)(F)CC1] |
| 267 | 2 | 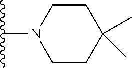 [SMILES: *N1CCC(C)(C)CC1] |
| 268 | 2 | 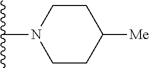 [SMILES: *N1CCC(C)CC1] |
| 269 | 2 | 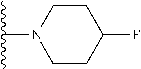 [SMILES: *N1CCC(F)CC1] |
| 270 | 2 | 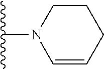 [SMILES: *N1C=CCCC1] |
| 271 | 2 | 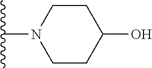 [SMILES: *N1CCC(O)CC1] |
| 272 | 2 | 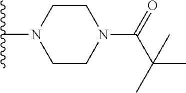 [SMILES: *N1CCN(C(=O)C(C)(C)C)CC1] |
| 273 | 2 | 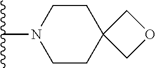 [SMILES: *N1CCC2(CC1)COC2] |
| 274 | 2 | 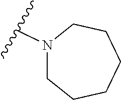 [SMILES: *N1CCCCCC1] |
| 275 | 2 | 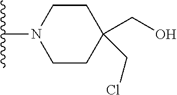 [SMILES: *N1CCC(CO)(CCl)CC1] |
| 276 | 2 | 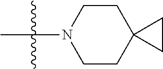 [SMILES: *N1CCC2(CC1)CC2] |
| 277 | 2 | 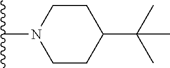 [SMILES: *N1CCC(C(C)(C)C)CC1] |
| 278 | 2 | 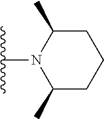 [SMILES: *N1[C@H](C)CCC[C@@H]1C] |
| 279 | 2 | 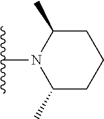 [SMILES: *N1[C@@H](C)CCC[C@@H]1C] |
| 280 | 2 | 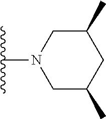 [SMILES: *N1C[C@H](C)C[C@H](C)C1] |
| 281 | 2 | 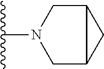 [SMILES: *N1CC2CC2C1] |
| 282 | 2 | 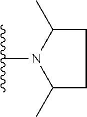 [SMILES: *N1C(C)CCC1C] |
| 283 | 2 | 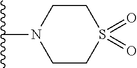 [SMILES: *N1CCS(=O)(=O)CC1] |
| 284 | 2 | 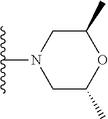 [SMILES: *N1C[C@@H](C)O[C@H](C)C1] |
| 285 | 2 | 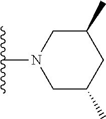 [SMILES: *N1C[C@@H](C)C[C@H](C)C1] |
| 286 | 2 | 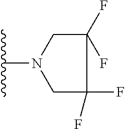 [SMILES: *N1CC(F)(F)C(F)(F)C1] |
| 287 | 2 | 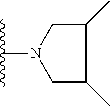 [SMILES: *N1CC(C)C(C)C1] |
| 288 | 2 | 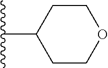 [SMILES: *C1CCOCC1] |
| 289 | 2 | 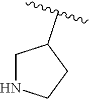 [SMILES: *C1CCNC1] |
| 290 | 2 | 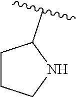 [SMILES: *C1CCCN1] |
| 291 | 2 | 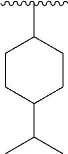 [SMILES: *C1CCC(C(C)C)CC1] |
| 292 | 2 | 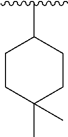 [SMILES: *C1CCC(C)(C)CC1] |
| 293 | 2 | 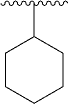 [SMILES: *C1CCCCC1] |
| 294 | 2 | 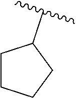 [SMILES: *C1CCCC1] |
| 295 | 2 | 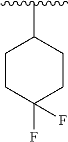 [SMILES: *C1CCC(F)(F)CC1] |
| 296 | 2 | 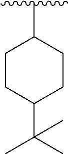 [SMILES: *C1CCC(C(C)(C)C)CC1] |
| 297 | 2 | 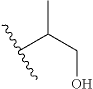 [SMILES: *C(C)CO] |
| 298 | 2 | 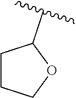 [SMILES: *C1CCCO1] |
| 299 | 2 | 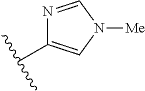 [SMILES: *c1cn(C)cn1] |
| 300 | 2 | 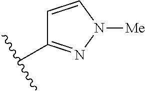 [SMILES: *c1ccn(C)n1] |
| 301 | 2 | 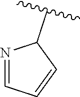 [SMILES: *C1C=CC=N1] |
| 302 | 2 | 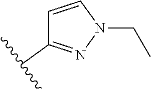 [SMILES: *c1ccn(CC)n1] |
| 303 | 2 | 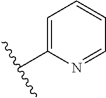 [SMILES: *c1ccccn1] |
| 304 | 2 | 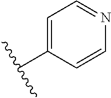 [SMILES: *c1ccncc1] |
| 305 | 2 | 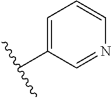 [SMILES: *c1cccnc1] |
| 306 | 2 | 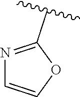 [SMILES: *c1ncco1] |
| 307 | 2 | 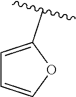 [SMILES: *c1ccco1] |
| 308 | 2 | 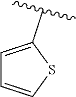 [SMILES: *c1cccs1] |
| 309 | 2 | 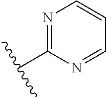 [SMILES: *c1ncccn1] |
| 310 | 2 | 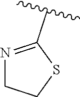 [SMILES: *C1=NCCS1] |
| 311 | 2 | 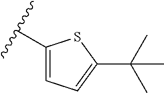 [SMILES: *c1ccc(C(C)(C)C)s1] |
| 312 | 2 | 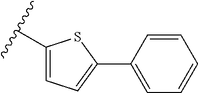 [SMILES: *c1ccc(-c2ccccc2)s1] |
| 313 | 2 | 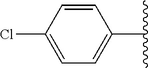 [SMILES: *c1ccc(Cl)cc1] |
| 314 | 2 | 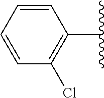 [SMILES: *c1ccccc1Cl] |
| 315 | 2 | 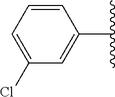 [SMILES: *c1cccc(Cl)c1] |
| 316 | 2 | 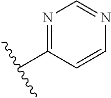 [SMILES: *c1ccncn1] |
| 317 | 2 | 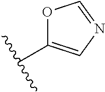 [SMILES: *c1cnco1] |
| 318 | 2 | 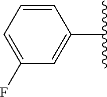 [SMILES: *c1cccc(F)c1] |
| 319 | 2 | 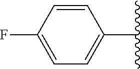 [SMILES: *c1ccc(F)cc1] |
| 320 | 2 | 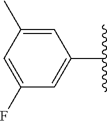 [SMILES: *c1cc(C)cc(F)c1] |
| 321 | 2 | 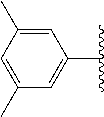 [SMILES: *c1cc(C)cc(C)c1] |
| 322 | 2 | 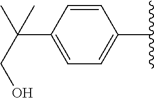 [SMILES: *c1ccc(C(C)(C)CO)cc1] |
| 323 | 2 | 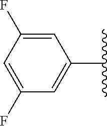 [SMILES: *c1cc(F)cc(F)c1] |
| 324 | 2 | 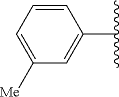 [SMILES: *c1cccc(C)c1] |
| 325 | 2 | 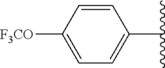 [SMILES: *c1ccc(OC(F)(F)F)cc1] |
| 326 | 2 | 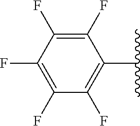 [SMILES: *c1c(F)c(F)c(F)c(F)c1F] |
| 327 | 2 | 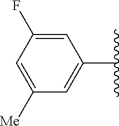 [SMILES: *c1cc(C)cc(F)c1] |
| 328 | 2 | 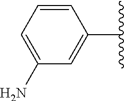 [SMILES: *c1cccc(N)c1] |
| 329 | 2 | 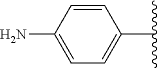 [SMILES: *c1ccc(N)cc1] |
| 330 | 2 | 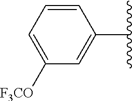 [SMILES: *c1cccc(OC(F)(F)F)c1] |
| 331 | 2 | 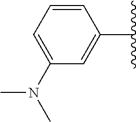 [SMILES: *c1cccc(N(C)C)c1] |
| 332 | 2 | 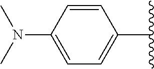 [SMILES: *c1ccc(N(C)C)cc1] |
| 333 | 2 | 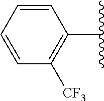 [SMILES: *c1ccccc1C(F)(F)F] |
| 334 | 2 | 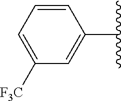 [SMILES: *c1cccc(C(F)(F)F)c1] |
| 335 | 2 | 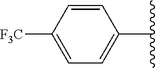 [SMILES: *c1ccc(C(F)(F)F)cc1] |
| 336 | 2 | 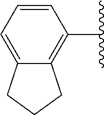 [SMILES: *c1cccc2c1CCC2] |
| 337 | 2 | 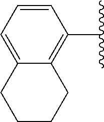 [SMILES: *c1cccc2c1CCCC2] |
| 338 | 2 | 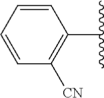 [SMILES: *c1ccccc1C#N] |
| 339 | 2 | 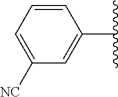 [SMILES: *c1cccc(C#N)c1] |
| 340 | 2 | 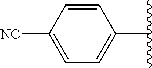 [SMILES: *c1ccc(C#N)cc1] |
| 341 | 2 | 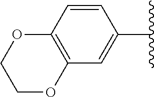 [SMILES: *c1ccc2c(c1)OCCO2] |
| 342 | 2 | 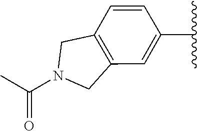 [SMILES: *c1ccc2c(c1)CN(C(C)=O)C2] |
| 343 | 2 | 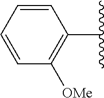 [SMILES: *c1ccccc1OC] |
| 344 | 2 | 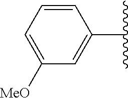 [SMILES: *c1cccc(OC)c1] |
| 345 | 2 | 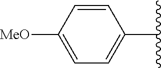 [SMILES: *c1ccc(OC)cc1] |
| 346 | 2 | 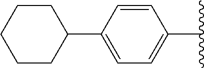 [SMILES: *c1ccc(C2CCCCC2)cc1] |
| 347 | 2 | 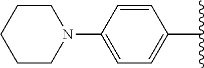 [SMILES: *c1ccc(N2CCCCC2)cc1] |
| 348 | 2 | 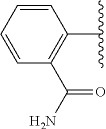 [SMILES: *c1ccccc1C(N)=O] |
| 349 | 2 | 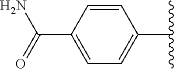 [SMILES: *c1ccc(C(N)=O)cc1] |
| 350 | 2 | 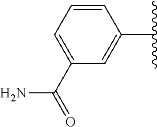 [SMILES: *c1cccc(C(N)=O)c1] |
| 351 | 2 | 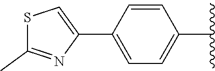 [SMILES: *c1ccc(-c2csc(C)n2)cc1] |

Various additional embodiments of R₁, R₃, R₄, R₅, R₇, R₈, R₉, R₁₀, R₁₁, R₁₂, Rₐ, R_{b}, etc. are described in WO 2017/147174 (e.g., as R₇, R₁, R₂, R₃, R₅, R₆, R₈, R₉, R₁₀, R₁₁, Rₐ, R_{b}, etc., respectively) either individually or in combination (e.g., as in compounds).

In some embodiments, a provided compound is a compound described in WO 2017/147174, wherein its 3-groups attached to moiety A are replaced with R¹ and R^{1a}, e.g., in some embodiments, both replaced with —H.

In some embodiments, a provided compound is a compound selected from compounds 1-348 of WO 2017/147174, wherein its 3-OH and 3-H attached to moiety A are replaced with R¹ and R^{1a}, e.g., in some embodiments, both replaced with —H.

In some embodiments, a provided compound is a compound selected from compounds in Schemes 1-9, Examples 1-7, and Tables 1-4 and Tables 1-2 in the Examples of WO 2017/147174, wherein its 3-groups (e.g., 3-OH/protected OH and 3-H attached to moiety A) are replaced with R¹ and R^{1a}, for example, in some embodiments, both replaced with —H. In some embodiments, a provided compound is a claimed compound in WO 2017/147174 or its corresponding national applications wherein its 3-groups (e.g., 3-OH/protected OH and 3-H attached to moiety A) are replaced with R¹ and R^{1a}, for example, in some embodiments, both replaced with —H.

In some embodiments, a compound is a compound in Table 2 of WO 2017/147174, wherein the compound has an EC50 labeled as “A” and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound in Table 2 of WO 2017/147174, wherein the compound has an EC50 labeled as “B” and wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2018/102418, e.g., a compound of formula (I) or a salt thereof (including stereoisomer, solvate, hydrate or combination thereof) as described in WO 2018/102418, wherein —OR₄ in WO 2018/102418 (in various embodiments, R₄ is —OH in WO 2018/102418) is replaced with R¹ or R^{a} as described herein. In some embodiments, —OR₄ in WO 2018/102418 and the —H that is attached to the same carbon as —OR₄ are replaced with R¹ and R^{1a} as described herein.

In some embodiments, a provided compound is a compound of formula IX or a salt thereof:

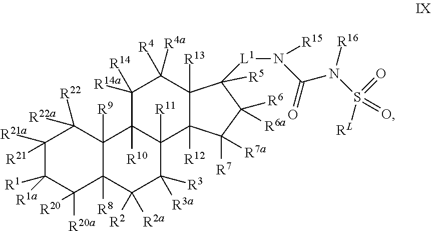

wherein each variable is independently as described herein. In some embodiments, each of R¹⁵ and R¹⁶ is independently R′ as described herein. In some embodiments, each of R¹⁵ and R¹⁶ is independently R as described herein. In some embodiments, each of them is —H.

In some embodiments, a provided compound is a compound of formula X or a salt thereof:

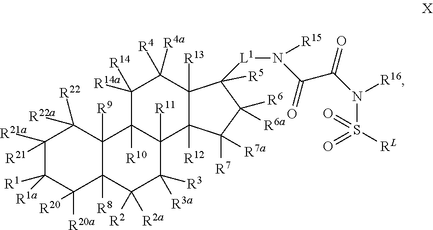

wherein each variable is independently as described herein. In some embodiments, each of R¹⁵ and R¹⁶ is independently R′ as described herein. In some embodiments, each of R¹⁵ and R¹⁶ is independently R as described herein. In some embodiments, each of them is —H.

In some embodiments, one of R² and R^{2a} is —H and the other is R₈ as described herein (e.g., in some embodiments, R₈ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R¹ is optionally substituted C₁₋₆ aliphatic. In some embodiments, R is optionally substituted C₁₋₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{a}-L^{b}-L^{c}-, wherein each of L^{a} and L^{c} is independently a covalent bond, or an optionally substituted bivalent C₁ aliphatic or heteroaliphatic group having 1-10 heteroatoms wherein a methylene unit of the group is optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —CC(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)N(R′)S(O)₂—, —N(R′)C(O)O—, —S(O)—, —S(O)₂—, —S(O)₂N(R′)—, —P(O)(OR′)—, or —P(O)(OR′)O—, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, a methylene unit bonded to L^{c} is replaced with —C(R′)₂—. In some embodiments, a methylene unit bonded to L^{c} is replaced with —CHR′—. In some embodiments, R′ is R₃ as described herein. In some embodiments, L^{b} is —(CH₂)m-CH(R′)— as described herein. In some embodiments, L^{b} is —(CH₂)m-CH(R₃)— as described herein. In some embodiments, —(CH₂)m- is bonded to L^{a}. In some embodiments, L^{b} is optionally substituted —CH₂CH₂—. In some embodiments, L^{b} is —CH₂CH₂—. In some embodiments, L^{c} is a covalent bond. In some embodiments, L^{c} is —N(R′)—. In some embodiments, L^{c} is —NH—. In some embodiments, L^{c} is —N(R′)C(O)—. In some embodiments, L^{c} is —NHC(O)—. In some embodiments, L^{c} is —N(R′)C(O)N(R′)—. In some embodiments, L^{c} is —NHC(O)NH—. In some embodiments, L^{c} is —N(R′)C(O)N(R′)S(O)₂—. In some embodiments, L^{c} is —NHC(O)NHS(O)₂—. In some embodiments, —S(O)₂— is bonded to R^{L}. In some embodiments, —S(O)₂— is bonded to R₁.

In some embodiments, R^{L} is R^{s}. In some embodiments, R^{L} is R′. In some embodiments, R^{L} is R. In some embodiments, R^{L} is —H. In some embodiments, R^{L} is —C(O)R^{s}. In some embodiments, R^{L} is —S(O)₂R^{s}. In some embodiments, R^{L} is —N(R′)S(O)₂R^{s}. In some embodiments, R^{L} is —C(O)N(R′)S(O)₂R^{s}. In some embodiments, R^{L} is —N(R′)C(O)N(R′)S(O)₂R^{s}. In some embodiments, R^{s} is R₁ as described herein. In some embodiments, R^{L} is R₁ as described herein.

In some embodiments, a provided compound is a compound of formula K—I or a salt thereof:

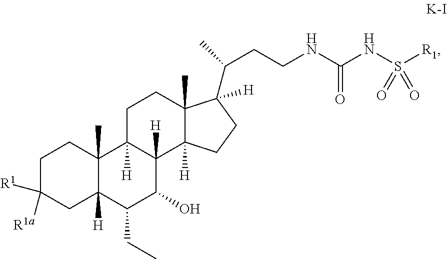

wherein each variable is independently as described herein.

In some embodiments, R₁ is —NR₉R₁₀, or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-12 membered heterocyclyl (e.g., having 1-5 heteroatoms), heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl); and
R₉ and R₁₀ are each independently —H, or an optionally substituted group selected from C₁₋₈ alkyl, C₂₋₈ alkenyl, C₂₋₈ alkynyl, and C₃₋₈ cycloalkyl, or R₉ and R₁₀ are taken together with the nitrogen to which they are attached to form an optionally substituted 3-15 membered (e.g., 3-12, 3-10, 3-9, 3-8, 3-7, 3-6, 3-5, etc., or 3-, 4-, 5-, 6-, 7-, 8-, 9-, 10-, 11-, 12-, 13-, 14- or 15-membered), monocyclic, bicyclic or polycyclic heterocyclyl ring having 1-5 heteroatoms.

In some embodiments, a provided compound is a compound of formula K-II or a salt thereof:

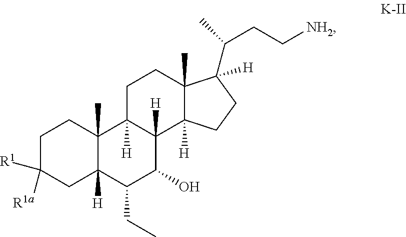

wherein each variable is independently as described herein.

In some embodiments, a provided compound is a compound of formula K—III or a salt thereof:

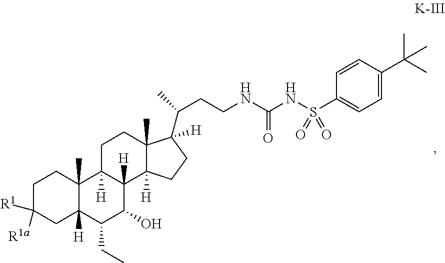

wherein each variable is independently as described herein.

In some embodiments, a provided compound is

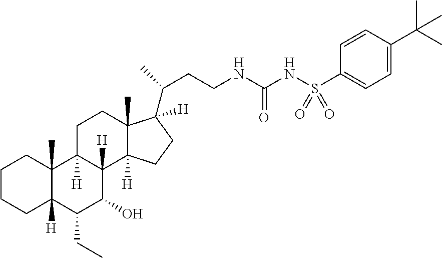

or salt thereof. In some embodiments, a provided compound is

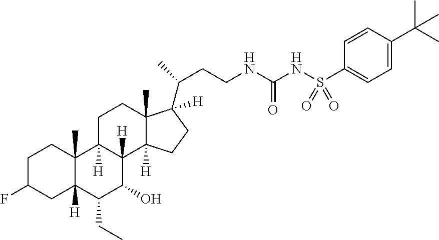

or salt thereof. In some embodiments, a provided compound is

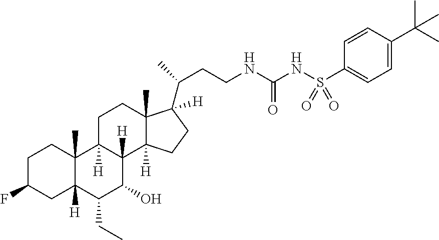

or salt thereof. In some embodiments, a provided compound is

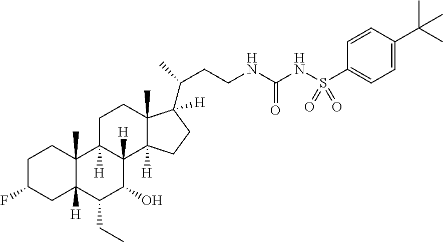

or salt thereof. In some embodiments, a provided compound is

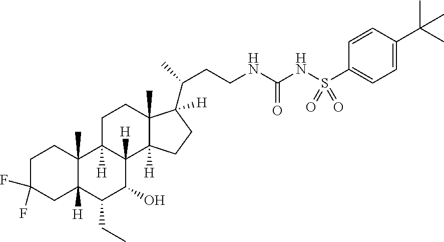

or salt thereof.

In some embodiments, a provided compound is

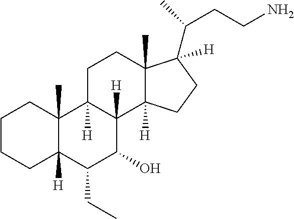

or salt thereof. In some embodiments, a provided compound is

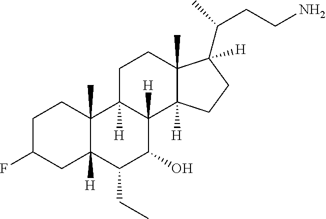

or salt thereof. In some embodiments, a provided compound is

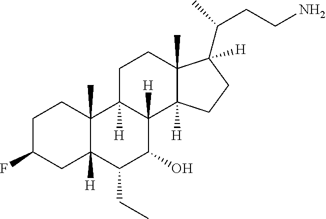

or salt thereof. In some embodiments, a provided compound is

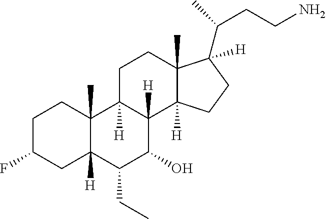

or salt thereof. In some embodiments, a provided compound is

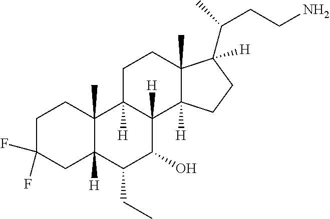

or salt thereof.

In some embodiments, a provided compound is a compound described in WO 2018/102418, wherein its 3-groups attached to moiety A are replaced with R¹ and R^{1a}, e.g., in some embodiments, both replaced with —H.

In some embodiments, a provided compound is a compound selected from compounds in Schemes 1-7, Examples 1-10, and Steps 1-8 of WO 2018/102418, wherein its 3-groups (e.g., 3-OH/protected OH and 3-H attached to moiety A) are replaced with R¹ and R^{1a}, for example, in some embodiments, both replaced with —H. In some embodiments, a provided compound is a compound in a claim in WO 2018/102418 or its corresponding national applications wherein its 3-groups (e.g., 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2018/152171, e.g., a compound of formula (I) or a salt thereof (including stereoisomer, solvate, hydrate or combination thereof) as described in WO 2018/152171, wherein R₃ₐ and R_{3b} in WO 2018/152171 (in various embodiments, one of R₃ₐ is —H and the other is —OH in WO 2018/152171) are replaced with R¹ and R^{1a} as described herein.

In some embodiments, one of R² and R^{2a} is —H and the other is Rₛ as described herein (e.g., in some embodiments, Rₛ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R is optionally substituted C₁₋₆ aliphatic. In some embodiments, R is optionally substituted C₁₋₆ alkyl. In some embodiments, R is optionally substituted methyl. In some embodiments, R is methyl. In some embodiments, R¹⁰ is —H. In some embodiments, R¹¹ is —H. In some embodiments, R¹² is —H.

In some embodiments, L¹ is -L^{a}-L^{b}-L^{c}-, wherein each of L^{a} and L^{c} is independently a covalent bond, or an optionally substituted bivalent C₁ aliphatic or heteroaliphatic group having 1-10 heteroatoms wherein a methylene unit of the group is optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)N(R′)S(O)₂—, —N(R′)C(O)O—, —S(O)—, —S(O)₂—, —S(O)₂N(R′)—, —P(O)(OR′)—, or —P(O)(OR′)O—, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is a covalent bond. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{b} is a covalent bond. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, a methylene unit bonded to L^{c} is replaced with —C(R′)₂—. In some embodiments, a methylene unit bonded to L^{c} is replaced with —CHR′—. In some embodiments, R′ is R₃ as described herein. In some embodiments, L^{b} is —(CH₂)m-CH(R′)— as described herein. In some embodiments, L^{b} is —(CH₂)m-CH(R₃)— as described herein. In some embodiments, —(CH₂)m- is bonded to L^{a}. In some embodiments, L^{b} is optionally substituted —CH₂CH₂—. In some embodiments, L^{b} is —CH₂CH₂—. In some embodiments, L^{b} is optionally substituted —CH₂—. In some embodiments, L^{b} is —CH₂—O—. In some embodiments, L^{b} is —CH₂—OC(O)—. In some embodiments, L^{b} is —CH₂—OC(O)N(R′)—. In some embodiments, L^{b} is —CH₂—OC(O)NH—. In some embodiments, L^{b} is —CH₂—OC(O)N(R′)S(O)₂—. In some embodiments, L^{b} is —CH₂—OC(O)NHS(O)₂—. In some embodiments, L^{c} is a covalent bond. In some embodiments, L^{c} is —O—. In some embodiments, L^{c} is —OC(O)—. In some embodiments, L^{c} is —OC(O)N(R′)—. In some embodiments, L^{c} is —OC(O)NH—. In some embodiments, L^{c} is —OC(O)N(R′)S(O)₂—. In some embodiments, L^{c} is —OC(O)NHS(O)₂—. In some embodiments, L^{c} is —S(O)₂—. In some embodiments, L^{c} is —N(R′)S(O)₂—. In some embodiments, L^{c} is —NHS(O)₂—. In some embodiments, L^{c} is —C(O)N(R′)S(O)₂—. In some embodiments, L^{c} is —C(O)NHS(O)₂—. In some embodiments, L^{c} is —OC(O)N(R′)S(O)₂—. In some embodiments, L^{c} is —OC(O)NHS(O)₂—. In some embodiments, —S(O)₂— is bonded to R^{L}. In some embodiments, —S(O)₂— is bonded to R₁.

In some embodiments, L¹ is —CH(CH₃)—CH₂—. In some embodiments, L¹ is —CH(CH₃)—CH₂—O—. In some embodiments, L¹ is —CH(CH₃)—CH₂—OC(O)—. In some embodiments, L′ is —CH(CH₃)—CH₂—OC(O)N(R′)—. In some embodiments, L¹ is —CH(CH₃)—CH₂—OC(O)NH—. In some embodiments, L¹ is —CH(CH₃)—CH₂—OC(O)N(R′)S(O)₂—. In some embodiments, L¹ is —CH(CH₃)—CH₂—OC(O)NHS(O)₂—. In some embodiments, L¹ is (S)—CH(CH₃)—CH₂—. In some embodiments, L¹ is (S)—CH(CH₃)—CH₂—O—. In some embodiments, L¹ is (S)—CH(CH₃)—CH₂—OC(O)—. In some embodiments, L¹ is (S)—CH(CH₃)—CH₂—OC(O)N(R′)—. In some embodiments, L¹ is (S)—CH(CH₃)—CH₂—OC(O)NH—. In some embodiments, L¹ is (S)—CH(CH₃)—CH₂—OC(O)N(R′)S(O)₂—. In some embodiments, L¹ is (S)—CH(CH₃)—CH₂—OC(O)NHS(O)₂—. In some embodiments, L¹ is (R)—CH(CH₃)—CH₂—. In some embodiments, L¹ is (R)—CH(CH₃)—CH₂—O—. In some embodiments, L¹ is (R)—CH(CH₃)—CH₂—OC(O)—. In some embodiments, L¹ is (R)—CH(CH₃)—CH₂—OC(O)N(R′)—. In some embodiments, L¹ is (R)—CH(CH₃)—CH₂—OC(O)NH—. In some embodiments, L¹ is (R)—CH(CH₃)—CH₂—OC(O)N(R′)S(O)₂—. In some embodiments, L¹ is (R)—CH(CH₃)—CH₂—OC(O)NHS(O)₂—. In some embodiments, —CH(CH₃)— is bonded to moiety A (i.e., the other end is bonded to R^{L}).

In some embodiments, R^{L} is R^{s}. In some embodiments, R^{L} is —S(O)₂R^{s}. In some embodiments, R^{L} is —N(R′)S(O)₂R^{s}. In some embodiments, R^{L} is —NHS(O)₂R^{s}. In some embodiments, R^{L} is —C(O)N(R′)S(O)₂R^{s}. In some embodiments, R^{L} is —C(O)NHS(O)₂R^{s}. In some embodiments, R^{L} is —OC(O)N(R′)S(O)₂R^{s}. In some embodiments, R^{L} is —OC(O)NHS(O)₂R^{s}.

In some embodiments, R^{s} is

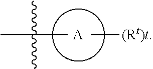

In some embodiments, R^{L} is

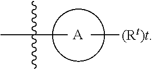

In some embodiments, R₁ is

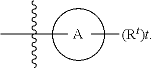

In some embodiments, t is 0. In some embodiments, t is 1. In some embodiments, t is 2. In some embodiments, t is 3. In some embodiments, t is 4. In some embodiments, t is 5. In some embodiments, t is 6. In some embodiments, Ring A is substituted in addition to —(R′)t. In some embodiments, Ring A is unsubstituted in addition to —(R)t. In some embodiments, Ring A is monocyclic. In some embodiments, Ring A is bicyclic. In some embodiments, Ring A is polycyclic. In some embodiments, Ring A is saturated. In some embodiments, Ring A is partially unsaturated. In some embodiments, Ring A is aromatic. In some embodiments, Ring A is an optionally substituted phenyl ring. In some embodiments, Ring A is an optionally substituted 5-membered heteroaryl ring having 1-4 heteroatoms. In some embodiments, Ring A is an optionally substituted 6-membered heteroaryl ring having 1-5 heteroatoms. In some embodiments, Ring A is bicyclic or polycyclic, wherein each monocyclic ring unit is independently an optionally substituted 3-10 (e.g., 3-, 4-, 5-, 6-, 7-, 8-, 9-, or 10-) membered saturated, partially unsaturated or aromatic ring having 1-6 (e.g., 1, 2, 3, 4, 5, or 6) heteroatoms. In some embodiments, at least one monocyclic ring unit is saturated. In some embodiments, each monocyclic ring unit is saturated. In some embodiments, at least one monocyclic ring unit is partially unsaturated. In some embodiments, each monocyclic ring unit is partially unsaturated. In some embodiments, at least one monocyclic ring unit is aromatic. In some embodiments, each monocyclic ring unit is aromatic. In some embodiments, at least one monocyclic ring unit is aromatic and at least one is partially unsaturated. In some embodiments, at least one monocyclic ring unit is aromatic and at least one is saturated. In some embodiments, at least one monocyclic ring unit is partially unsaturated and at least one is saturated.

In some embodiments, R^{s} (or R^{L}, R₁, etc. that can be R^{s}) is

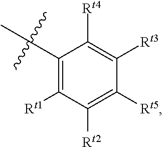

wherein each of R^{t1}, R^{t2}, R^{t3}, R^{t4} and R^{t5} is independently R^{t}, and R^{ta} is R′ as described herein. In some embodiments, R^{s} is

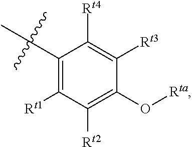

R^{t2}, wherein each of R^{t1}, R^{t2}, R^{t3}, and R^{t4} is independently R^{t}, and R^{ta} is R′ as described herein. In some embodiments, R^{ta} is —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl). In some embodiments, each of R^{t1}, R^{t2}, R^{t3} and R^{t4} is independently —H, halogen, or an optionally substituted C₁₋₈ alkyl. In some embodiments, R^{s} is

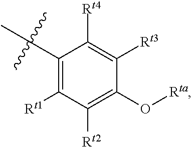

wherein R^{ta} is —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl), and each of R^{t1}, R^{t2}, R^{t3} and R^{t4} is independently —H, halogen, or an optionally substituted C₁₋₈ alkyl.

In some embodiments, R^{s} (or R^{L}, R₁, etc. that can be R^{s}) is

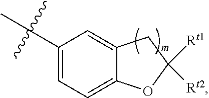

wherein each of m, R^{t1} and R^{t2} is independently as described herein. In some embodiments, R^{s} is

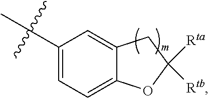

wherein each of R^{ta} and R^{tb} is independently R′ as described herein. In some embodiments, each of R^{ta} and R^{tb} is independently —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl), and m is 1, 2 or 3.

In some embodiments, R^{s} (or R^{L}, R₁, etc. that can be R^{s}) is

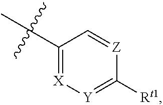

wherein R^{t1} is as described herein, and each of X, Y and Z is independently —C(R^{t})═ or —N═. In some embodiments, R^{s} is

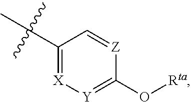

wherein R^{ta} is R′ as described herein. In some embodiments, R^{ta} is —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl), each of X, Y and Z is independently —C(R)═ wherein R^{t} is —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl) or —N=. In some embodiments, at least one of X, Y and Z is —N=.

In some embodiments, a provided compound is a compound of formula XIII-a or a salt thereof:

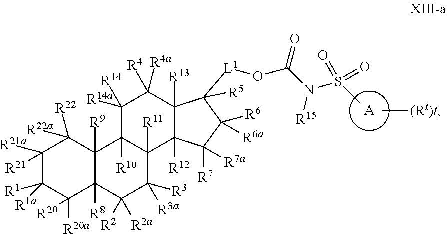

wherein each variable is independently as described herein. In some embodiments, R¹⁵ is R′ as described herein. In some embodiments, R¹⁵ is R as described herein. In some embodiments, R¹⁵ is —H.

In some embodiments, a provided compound is a compound of formula L-I or a salt thereof:

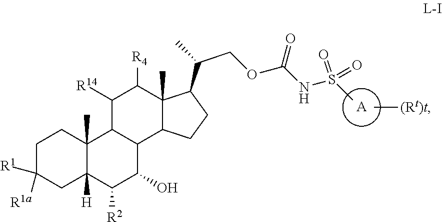

wherein each variable is independently as described herein.

In some embodiments, a provided compound is a compound of formula L-Ia or a salt thereof:

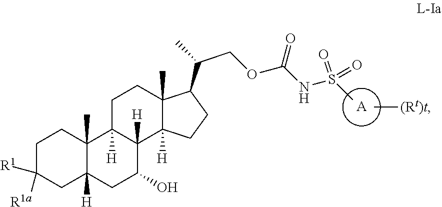

wherein each variable is independently as described herein.

In some embodiments, a provided compound is a compound of formula L-Ib or a salt thereof:

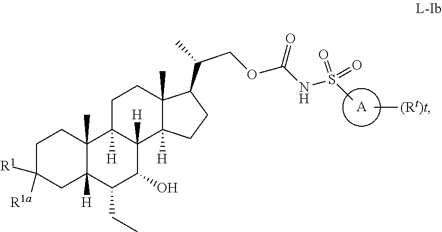

wherein each variable is independently as described herein.

In some embodiments, R^{s} (or R^{L}, R₁, etc. that can be R^{s}) is

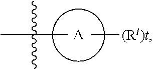

wherein

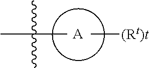

is

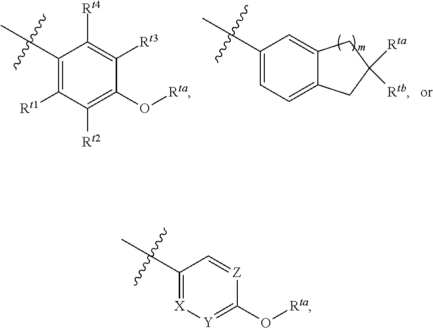

and:
each of R^{ta} and R^{tb} is independently —H or an optionally substituted group selected from C₁₋₈ alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-8 membered heterocycloalkyl (e.g., having 1-5 heteroatoms), heteroaryl (e.g., 5-14 membered heteroaryl having 1-10 heteroatoms) and heteroarylalkyl (e.g., (5-14 membered heteroaryl having 1-10 heteroatoms)C₁₋₁₂alkyl);
each of R^{t1}, R^{t2}, R^{t3} and R^{t4} is independently —H, halogen or optionally substituted C₁₋₈ alkyl;
m is 1, 2 or 3;
each of X, Y and Z is independently —C(R^{t1})═ or —N═, preferably at least one of X, Y and Z is —N═;
R⁴ is —H, —OH, —OSO₃H, —OAc, —OPO₃H₂, or optionally substituted C₁₋₆ alkyl; preferably R₁ is —H;
R¹⁴ is —H, halogen, —CN, —N₃, —OH, —OSO₃H, —OAc, —OPO₃H₂, —SR^{ta}, or —NHR^{ta}, or C₁₋₆ alkyl; preferably R¹⁴ is —H; or R⁴ and R¹⁴ are taken together with the carbon atoms to which they are attached to form —CH═CH— or an optionally substituted 3-15 membered, monocyclic, bicyclic or polycyclic ring having, in addition to the intervening atoms, 0-5 heteroatoms (e.g., cyclopropyl or epoxide);
R² is —H or ethyl; and
each of R¹ and R^{1a} is independently as described herein.

In some embodiments, R⁴ is —H. In some embodiments, R¹⁴ is —H. In some embodiments, both of R⁴ and R¹⁴ are —H.

In some embodiments, R² is —H. In some embodiments, R² is optionally substituted C₁₋₆ alkyl. In some embodiments, R² is ethyl.

In some embodiments, one of R⁴ and R¹⁴ is —H and R² is —H or ethyl. In some embodiments, R⁴ and R¹⁴ are —H and R² is —H or ethyl. In some embodiments, R⁴ and R¹⁴ are —H, R¹ and R^{1a} are —H and R² is —H or ethyl. In some embodiments, R⁴ and R¹⁴ are —H, one of R¹ and R^{1a} is —F and R² is —H or ethyl.

In some embodiments, R^{t}a is R. In some embodiments, R^{t}a is optionally substituted C₁₋₆ aliphatic. In some embodiments, R^{ta} is optionally substituted C₁₋₆ alkyl. In some embodiments, R^{ta} is —H. In some embodiments, R^{ta} is optionally substituted phenyl. In some embodiments, R^{ta} is t-butyl, —CF₃, —H, —CH₃ or -Ph. In some embodiments, R^{ta} is not t-butyl, —CF₃, —H, —CH₃ or -Ph.

In some embodiments, R^{ta} is an optionally substituted group selected from C₁₋₄ alkyl, C₂₋₄ alkenyl, C₃₋₆ cycloalkyl, 3-8 membered heterocyclyl (e.g., having 1-5 heteroatoms), aryl (e.g., C₆₋₁₄ aryl) and heteroaryl (e.g., 5-14 membered heteroaryl having 1-10 heteroatoms). In some embodiments, R^{ta} is —H, methyl, ethyl, propyl, isopropyl, butyl, 2-butyl, t-butyl, 3-pentyl, vinyl, ally, —CF₃, cyclopropyl, cyclobutyl, cyclopentyl, cyclohexyl, phenyl, 1-methylcyclopropyl, cyclopropylmethyl, 1-methylcyclobutyl, 1-methylcyclopentyl, 1-methylcyclohexyl, or

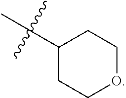 [SMILES: *C1CCOCC1]

In some embodiments,

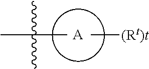

is

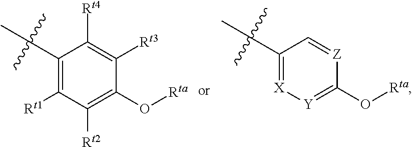

wherein R^{ta} is an optionally substituted group selected from C₁₋₄ alkyl, C₂₋₄ alkenyl, C₃₋₆ cycloalkyl, 3-8 membered heterocyclyl (e.g., having 1-5 heteroatoms), aryl (e.g., C₆₋₁₄ aryl) and heteroaryl (e.g., 5-14 membered heteroaryl having 1-10 heteroatoms).

In some embodiments,

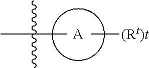

is

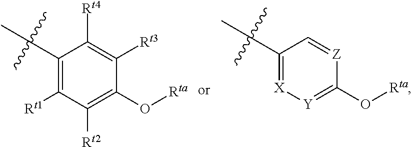

wherein R^{ta} is —H, methyl, ethyl, propyl, isopropyl, butyl, 2-butyl, t-butyl, 3-pentyl, vinyl, ally, —CF₃, cyclopropyl, cyclobutyl, cyclopentyl, cyclohexyl, phenyl, 1-methylcyclopropyl, cyclopropylmethyl, 1-methylcyclobutyl, 1-methylcyclopentyl, 1-methylcyclohexyl, or

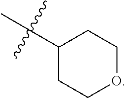 [SMILES: *C1CCOCC1]

In some embodiments,

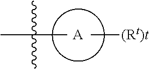

is

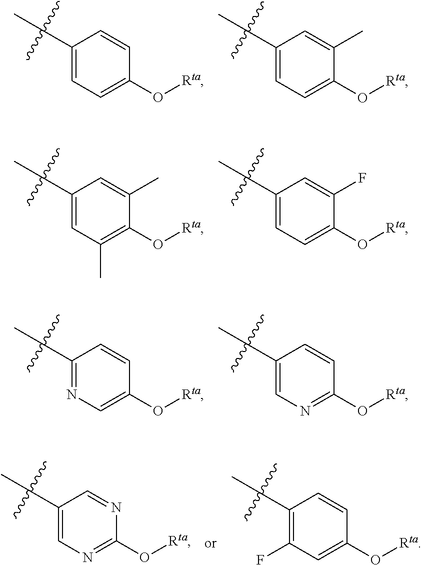

In some embodiments,

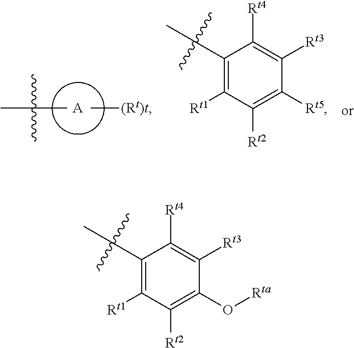

is

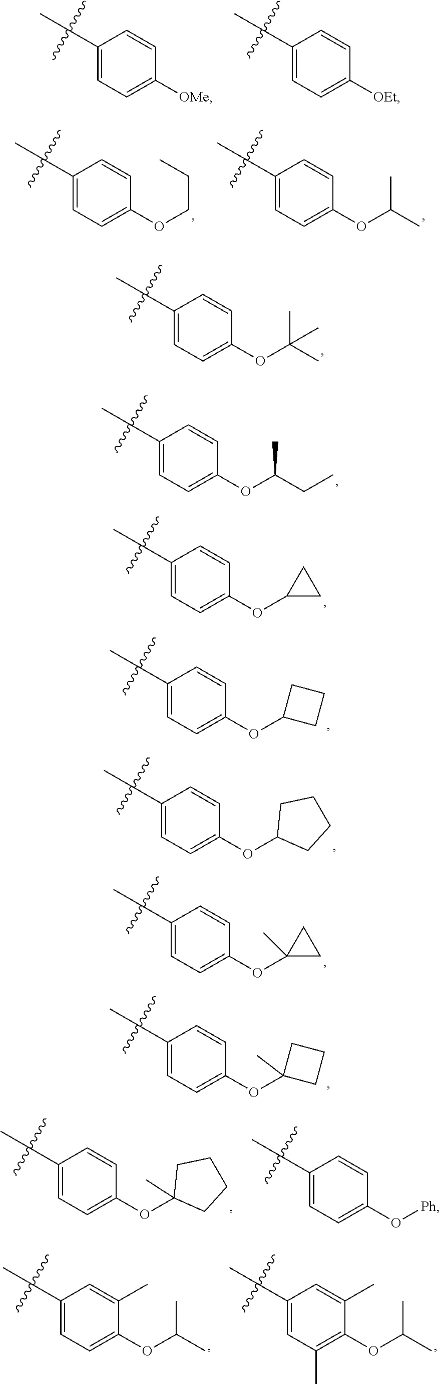

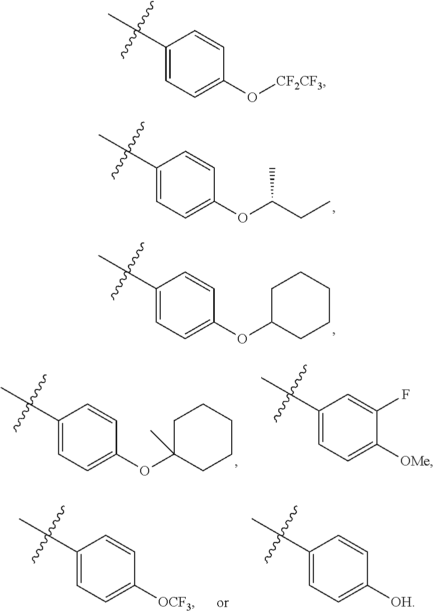

In some embodiments,

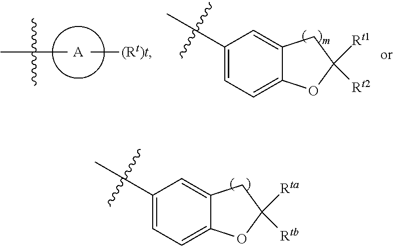

is

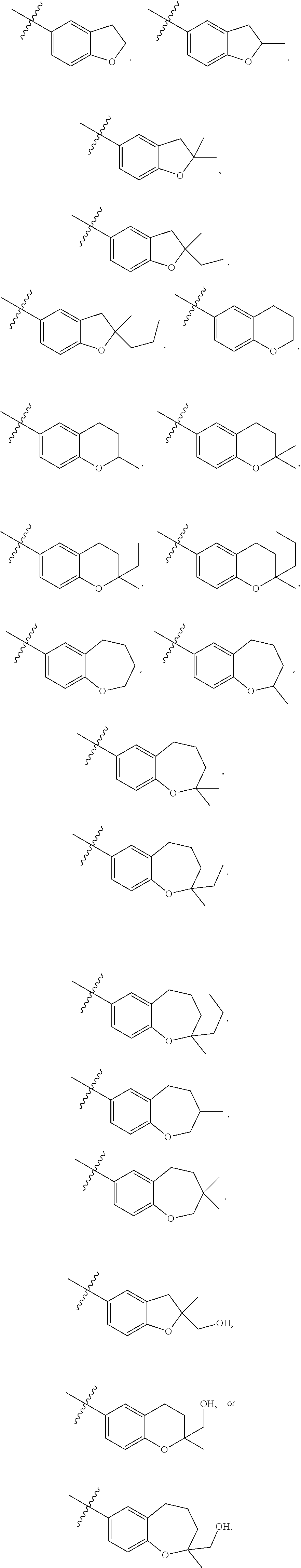

In some embodiments

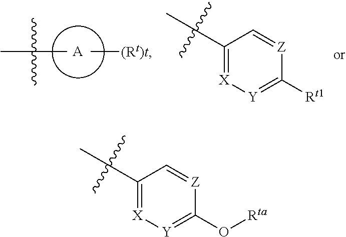

is

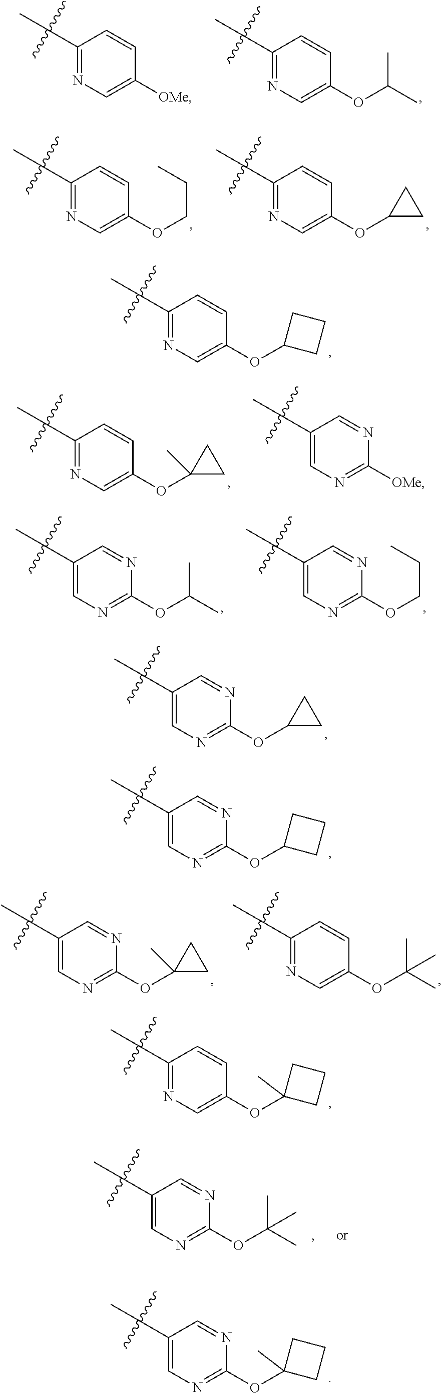

In some embodiments, a provided compound is a compound of formula L-II or a salt thereof:

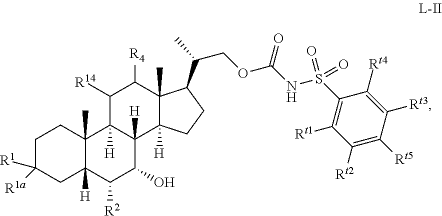

wherein each variable is independently as described herein.

In some embodiments, a provided compound is a compound of formula L-III or a salt thereof:

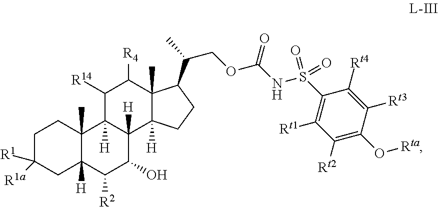

wherein each variable is independently as described herein.

In some embodiments, a provided compound is a compound of formula L-IV or a salt thereof:

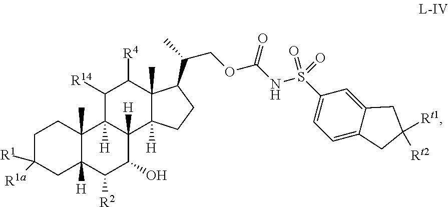

wherein each variable is independently as described herein.

In some embodiments, a provided compound is a compound of formula L-V or a salt thereof:

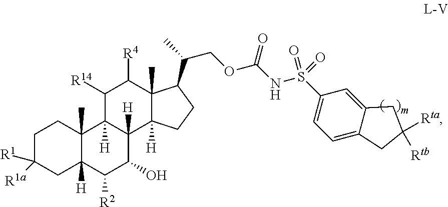

wherein each variable is independently as described herein.

In some embodiments, R⁴ and R¹⁴ are —H and R² is ethyl. In some embodiments, each of R^{t1}, R^{t2}, R^{t3} and R^{t4} are each independently —H or —CH₃. In some embodiments, each of R^{t1}, R^{t2}, R^{t3} and R^{t4} are —H. In some embodiments, R^{ta} is an optionally substituted group selected from C₁₋₄ alkyl, C₂₋₄ alkenyl, C₃₋₆ cycloalkyl, heteroaryl (e.g., 5-6 or 5-14 membered heteroaryl having 1-10 heteroatoms), and aryl (e.g., C₆₋₁₄ aryl such as phenyl, naphthyl, etc.), R^{t1}, R^{t2}, R^{t3} and R^{t4} are each independently —H or optionally substituted C₁₋₄ alkyl, and R² is —H or ethyl.

In some embodiments, a provided compound is a compound described in WO 2018/152171, wherein its 3-groups (e.g., R₃ₐ and R_{3b}, in some embodiments, —H and —OH) attached to moiety A are replaced with R¹ and R^{1a}, e.g., in some embodiments, both replaced with —H.

In some embodiments, a provided compound is a compound selected from compounds in Schemes 1-2 and Examples 1-34 in WO 2018/152171, wherein its 3-groups (e.g., 3-OH/protected OH and 3-H, or ═O attached to moiety A) are replaced with R¹ and R^{1a}, for example, in some embodiments, both replaced with —H. In some embodiments, a provided compound is a claimed compound in WO 2018/152171 or its corresponding national applications wherein its 3-groups (e.g., 3-OH/protected OH and 3-H attached to moiety A) are replaced with R¹ and R^{1a}, for example, in some embodiments, both replaced with —H.

In some embodiments, a compound is a compound of WO 2018/152171, wherein the compound has an EC50 labeled as “A” in its FXR agonistic assay and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound of WO 2018/152171, wherein the compound has an EC50 labeled as “B” in its FXR agonistic assay and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound of WO 2018/152171, wherein the compound has an EC50 labeled as “C” in its FXR agonistic assay and wherein its 3-groups (e.g., 3-groups such as 3-OH/protected OH and 3-H attached to moiety A) are replaced with R¹ and R^{1a}, for example, in some embodiments, both replaced with —H. In some embodiments, a compound is a compound of WO 2018/152171, wherein the compound has an EC50 labeled as “D” in its FXR agonistic assay and wherein its 3-groups (e.g., 3-groups such as 3-OH/protected OH and 3-H attached to moiety A) are replaced with R¹ and R^{1a}, for example, in some embodiments, both replaced with —H.

In some embodiments, a provided compound, e.g., a compound of formula I or a salt thereof, has the structure of a compound described in WO 2018/187804, e.g., a compound of formula (I) or a salt thereof (including stereoisomer, solvate, hydrate or combination thereof) as described in WO 2018/187804, wherein 3-OH in WO 2018/187804 and the —H attached to the same carbon as the —OH are replaced with R¹ and R^{1a} as described herein. In some embodiments, 3-OH in WO 2018/187804 is replaced with R¹. In some embodiments, 3-OH in WO 2018/187804 is replaced with R^{1a}.

In some embodiments, one of R² and R^{2a} is —H and the other is R₈ as described herein (e.g., in some embodiments, R₈ is ethyl). In some embodiments, one of R³ and R^{3a} is —H and the other is —OR₇ as described herein (e.g., in some embodiments, —OH). In some embodiments, one of R⁴ and R^{4a} is —H and the other is R₄ as described herein (e.g., in some embodiments, —H; in some embodiments, —OH; etc.). In some embodiments, one of R¹⁴ and R^{14a} is —H and the other is R₅ as described herein (e.g., in some embodiments, —H). In some embodiments, R⁵ is —H. In some embodiments, R⁶ is —H. In some embodiments, R^{6a} is —H. In some embodiments, R⁷ is —H. In some embodiments, R^{7a} is —H. In some embodiments, R⁸ is —H. In some embodiments, R⁹ is R as described herein (e.g., in some embodiments, methyl). In some embodiments, R¹³ is R as described herein (e.g., in some embodiments, methyl). In some embodiments, R is optionally substituted C₁₋₆ aliphatic. In some embodiments, R is optionally substituted C₁₋₆ alkyl. In some embodiments, R is optionally substituted methyl. In some embodiments, R is methyl. In some embodiments, R¹⁰ is —H. In some embodiments, R¹¹ is —H. In some embodiments, R¹² is —H. In some embodiments, R¹⁴ is —H. In some embodiments, R¹⁴ is —OR. In some embodiments, R¹⁴ is —OH.

In some embodiments, L¹ is -L^{a}-L^{b}-L^{c}-, wherein each of L^{a} and L^{c} is independently a covalent bond, or an optionally substituted bivalent C₁ aliphatic or heteroaliphatic group having 1-10 heteroatoms wherein a methylene unit of the group is optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)N(R′)S(O)₂—, —N(R′)C(O)O—, —S(O)—, —S(O)₂—, —S(O)₂N(R′)—, —P(O)(OR′)—, or —P(O)(OR′)O—, and L^{b} is a covalent bond, or an optionally substituted, bivalent C₁₋₁₀ aliphatic or heteroaliphatic group having 1-6 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —S(O)—, —S(O)₂—, —S(O)₂N(R′)—, —P(O)(OR′)—, or —P(O)(OR′)O—. In some embodiments, L^{a} is a covalent bond. In some embodiments, L^{a} is optionally substituted —CH₂—. In some embodiments, L^{a} is —C(R′)₂—, In some embodiments, L^{a} is —CHR′—. In some embodiments, L^{a} is —CH(CH₃)—. In some embodiments, L^{a} is —(S)—CH(CH₃)—. In some embodiments, L^{a} is —(R)—CH(CH₃)—. In some embodiments, L^{b} is a covalent bond. In some embodiments, L^{b} is optionally substituted bivalent C₁₋₁₀ aliphatic. In some embodiments, L^{b} is C₁₋₁₀ is optionally substituted alkylene. In some embodiments, L^{b} is optionally substituted —(CH₂)₁₋₁₀—. In some embodiments, L^{b} is —(CH₂)m- as described herein. In some embodiments, a methylene unit bonded to L^{c} is replaced with —C(R′)₂—. In some embodiments, a methylene unit bonded to L^{c} is replaced with —CHR′—. In some embodiments, R′ is R₃ as described herein. In some embodiments, L^{b} is —(CH₂)m-CH(R′)— as described herein. In some embodiments, L^{b} is —(CH₂)m-CH(R₃)— as described herein. In some embodiments, —(CH₂)m- is bonded to L^{a}. In some embodiments, L^{b} is optionally substituted —CH₂CH₂—. In some embodiments, L^{b} is —CH₂CH₂—. In some embodiments, L^{b} is optionally substituted —CH₂—. In some embodiments, L^{b} is —CH₂—O—. In some embodiments, L^{b} is —CH₂—OC(O)—. In some embodiments, L^{b} is —CH₂—OC(O)N(R′)—. In some embodiments, L^{b} is —CH₂—OC(O)NH—. In some embodiments, L^{b} is —CH₂—OC(O)N(R′)S(O)₂—. In some embodiments, L^{b} is —CH₂—OC(O)NHS(O)₂—. In some embodiments, L^{c} is a covalent bond. In some embodiments, L^{c} is —O—. In some embodiments, L^{c} is —OC(O)—. In some embodiments, L^{c} is —OC(O)N(R′)—. In some embodiments, L^{c} is —OC(O)NH—. In some embodiments, L^{c} is —OC(O)N(R′)S(O)₂—. In some embodiments, L^{c} is —OC(O)NHS(O)₂—. In some embodiments, L^{c} is —S(O)₂—. In some embodiments, L^{c} is —N(R′)S(O)₂—. In some embodiments, L^{c} is —NHS(O)₂—. In some embodiments, L^{c} is —C(O)N(R′)S(O)₂—. In some embodiments, L^{c} is —C(O)NHS(O)₂—. In some embodiments, L^{c} is —OC(O)N(R′)S(O)₂—. In some embodiments, L^{c} is —OC(O)NHS(O)₂—. In some embodiments, —S(O)₂— is bonded to R^{L}. In some embodiments, —S(O)₂— is bonded to R₁.

In some embodiments, L¹ is —CH(CH₃)—CH₂—. In some embodiments, L¹ is —CH(CH₃)—CH₂—O—. In some embodiments, L¹ is —CH(CH₃)—CH₂—OC(O)—. In some embodiments, L¹ is —CH(CH₃)—CH₂—OC(O)N(R′)—. In some embodiments, L¹ is —CH(CH₃)—CH₂—OC(O)NH—. In some embodiments, L¹ is —CH(CH₃)—CH₂—OC(O)N(R′)S(O)₂—. In some embodiments, L¹ is —CH(CH₃)—CH₂—OC(O)NHS(O)₂—. In some embodiments, L¹ is (S)—CH(CH₃)—CH₂—. In some embodiments, L¹ is (S)—CH(CH₃)—CH₂—O—. In some embodiments, L¹ is (S)—CH(CH₃)—CH₂—OC(O)—. In some embodiments, L¹ is (S)—CH(CH₃)—CH₂—OC(O)N(R′)—. In some embodiments, L¹ is (S)—CH(CH₃)—CH₂—OC(O)NH—. In some embodiments, L¹ is (S)—CH(CH₃)—CH₂—OC(O)N(R′)S(O)₂—. In some embodiments, L¹ is (S)—CH(CH₃)—CH₂—OC(O)NHS(O)₂—. In some embodiments, L¹ is (R)—CH(CH₃)—CH₂—. In some embodiments, L¹ is (R)—CH(CH₃)—CH₂—O—. In some embodiments, L¹ is (R)—CH(CH₃)—CH₂—OC(O)—. In some embodiments, L¹ is (R)—CH(CH₃)—CH₂—OC(O)N(R′)—. In some embodiments, L¹ is (R)—CH(CH₃)—CH₂—OC(O)NH—. In some embodiments, L¹ is (R)—CH(CH₃)—CH₂—OC(O)N(R′)S(O)₂—. In some embodiments, L¹ is (R)—CH(CH₃)—CH₂—OC(O)NHS(O)₂—. In some embodiments, —CH(CH₃)— is bonded to moiety A (i.e., the other end is bonded to R^{L}).

In some embodiments, R^{L} is R^{s}. In some embodiments, R^{L} is —S(O)₂R^{s}. In some embodiments, R^{L} is —N(R′)S(O)₂R^{s}. In some embodiments, R^{L} is —NHS(O)₂R^{s}. In some embodiments, R^{L} is —C(O)N(R′)S(O)₂R^{s}. In some embodiments, R^{L} is —C(O)NHS(O)₂R^{s}. In some embodiments, R^{L} is —OC(O)N(R′)S(O)₂R^{s}. In some embodiments, R^{L} is —OC(O)NHS(O)₂R^{s}.

In some embodiments, a provided compound is a compound of formula XIII-b or a salt thereof:

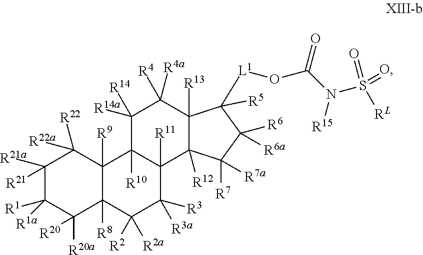

wherein each variable is independently as described herein.

In some embodiments, a provided compound is a compound of formula M-I or a salt thereof:

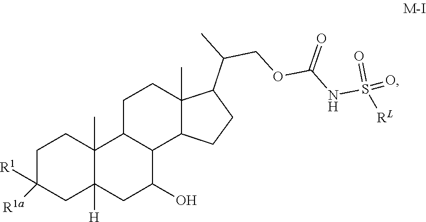

wherein each variable is independently as described herein.

In some embodiments, a provided compound is a compound of formula M-II or a salt thereof:

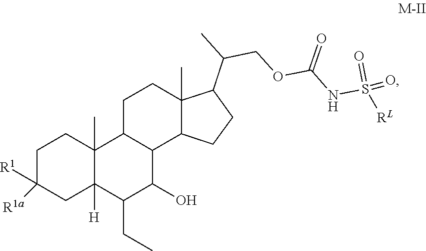

wherein each variable is independently as described herein.

In some embodiments, a provided compound is a compound of formula M-I′ or a salt thereof:

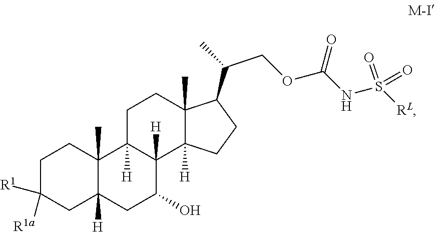

wherein each variable is independently as described herein.

In some embodiments, a provided compound is a compound of formula M-II′ or a salt thereof:

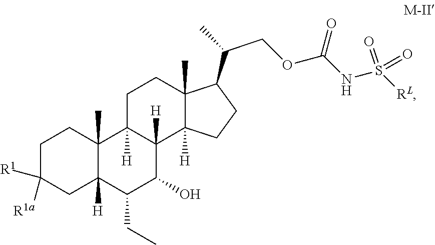

wherein each variable is independently as described herein.

In some embodiments, R^{L} is an optionally substituted group selected from 1-8 alkyl, C₂₋₈ alkenyl, C₂₋₈ alkynyl, C₃₋₈ cycloalkyl, aryl (e.g., C₆₋₁₄ aryl), arylalkyl (e.g., C₆₋₁₄arylC₁₋₁₂alkyl), 3-12 membered heterocycloalkyl, heteroaryl (e.g., 5-14 membered heteroaryl having 1-10 heteroatoms), and heteroarylalkyl (e.g., (5-14 membered heteroaryl having 1-10 heteroatoms)C₁₋₁₂alkyl), or R^{L} is —NRₐR_{b}, wherein each of Rₐ and R_{b} is independently —H, or an optionally substituted group selected from C₁₋₈ alkyl, C₂₋₈ alkenyl, C₂₋₈ alkynyl, and C₃₋₈ cycloalkyl, or Rₐ and R_{b} are taken together with the nitrogen to which they are attached to form an optionally substituted 3-12 heterocyclic ring (e.g., having 1-5 heteroatoms). In some embodiments, R^{L} is optionally substituted aryl (e.g., C₆₋₁₄ aryl). In some embodiments, R^{L} is optionally substituted heteroaryl (e.g., 5-14 membered heteroaryl having 1-10 heteroatoms). In some embodiments, R^{L} is optionally substituted heterocyclyl (e.g., 3-15 membered heterocyclyl having 1-5 heteroatoms). In some embodiments, R^{L} is optionally substituted phenyl. In some embodiments, R^{L} is optionally substituted pyridyl. In some embodiments, R^{L} is optionally substituted

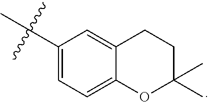 [SMILES: *c1ccc2c(c1)CCC(C)(C)O2]

In some embodiments, R^{L} is

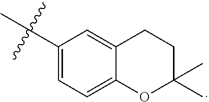 [SMILES: *c1ccc2c(c1)CCC(C)(C)O2]

In some embodiments, R^{L} is optionally substituted

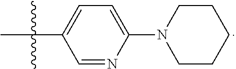 [SMILES: *c1ccc(N2CCCCC2)nc1]

In some embodiments, R^{L} is

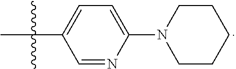 [SMILES: *c1ccc(N2CCCCC2)nc1]

In some embodiments, a provided compound is a compound of formula M-III or a salt thereof:

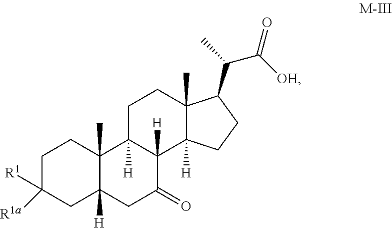

wherein each variable is independently as described herein.

In some embodiments, a provided compound is a compound of formula M-IV or a salt thereof:

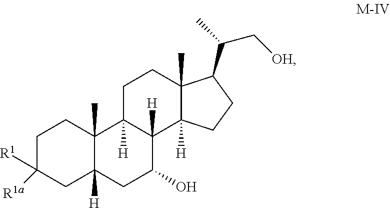

wherein each variable is independently as described herein.

In some embodiments, a provided compound is a compound of formula M-V or a salt thereof:

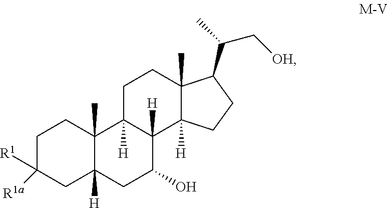

wherein each variable is independently as described herein.

In some embodiments, a provided compound is a compound of formula M-VI or a salt thereof:

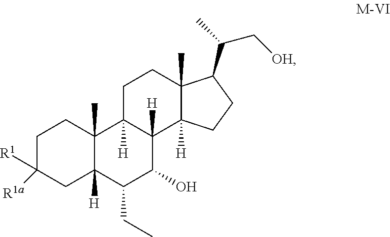

wherein each variable is independently as described herein.

In some embodiments, a provided compound is a compound of formula M-VII or a salt thereof:

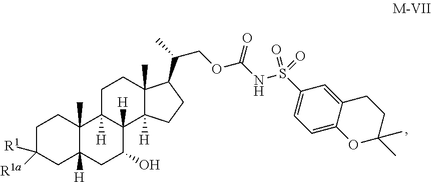

wherein each variable is independently as described herein.

In some embodiments, a provided compound is a compound of formula M-VIII or a salt thereof:

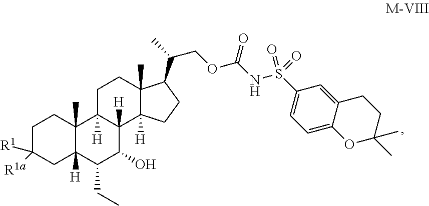

wherein each variable is independently as described herein.

In some embodiments, a provided compound is a compound of formula M-IX-a or a salt thereof:

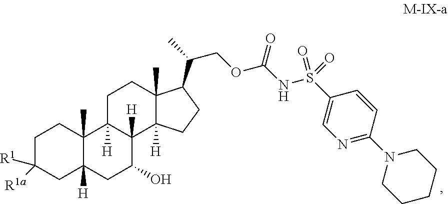

wherein each variable is independently as described herein.

In some embodiments, a provided compound is a compound of formula M-IX-b or a salt thereof:

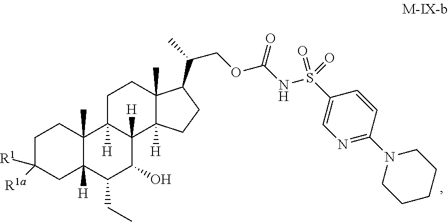

wherein each variable is independently as described herein.

In some embodiments, each of R¹ and R^{1a} is independently —H or halogen. In some embodiments, one of R¹ and R^{1a} is —H and the other is —H or halogen. In some embodiments, one of R¹ and R^{1a} is —H and the other is —H or —F. In some embodiments, R¹ and R^{1a} are —H. In some embodiments, each of R¹ and R^{1a} is independently halogen. In some embodiments, R¹ and R^{1a} are —F.

In some embodiments, a provided compound is a compound described in WO 2018/187804, wherein its 3-groups (e.g., —H and —OH) attached to moiety A are replaced with R¹ and R^{1a}, e.g., in some embodiments, both replaced with —H.

In some embodiments, a provided compound is a compound selected from compounds in Schemes 1-11 and Examples 1-25 in WO 2018/187804, wherein its 3-groups (e.g., 3-OH/protected OH and 3-H, or ═O attached to moiety A) are replaced with R¹ and R^{1a}, for example, in some embodiments, both replaced with —H. In some embodiments, a provided compound is a claimed compound in WO 2018/187804 or its corresponding national applications wherein its 3-groups (e.g., 3-OH/protected OH and 3-H attached to moiety A) are replaced with R¹ and R^{1a}, for example, in some embodiments, both replaced with —H.

In some embodiments, various compounds, e.g., those described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, WO 2016/173524, US20160145295A1, WO2017129125, Xian et al., ACS Med. Chem. Lett. 2017, 8, 1246-1251, et al., are modified (e.g., replacing 3-groups, e.g., 3-OH/protected OH attached to moiety A, with R^{1a} and R¹ as described herein) in accordance with the present disclosure.

### Production

In some embodiments, the present disclosure provides technologies for preparing provided compounds and compositions thereof. Those skilled in the art reading the present disclosure will appreciate that various synthetic technologies, e.g., certain technologies described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, WO 2016/173524, etc., can be utilized in accordance with the present disclosure. In some embodiments, manufacturing a provided compound comprises utilizing a bile acid as a starting material.

In some embodiments, the present disclosure provides a method comprising:
oxidizing a 3-OH group bonded to moiety A in a compound (e.g., a compound of formula I wherein one of R¹ or R^{1a} is —OH and the other is —H) or a salt thereof, and
producing a compound comprising a 3-ketone group bonded to moiety A (e.g., a compound of formula I wherein R¹ and R^{1a} are taken together to form ═O) or a salt thereof.
   In some embodiments, the two compounds are otherwise identical except that prior to oxidation, one of R¹ and R^{1a} is —OH and the other is —H (e.g., R¹ is —OH and R^{1a} is —H), while after oxidation, R¹ and R^{1a} are taken together to form ═O. Various oxidizing technologies are available to those skilled in the art and can be utilized in accordance with the present disclosure, for example, in some embodiments, an oxidation is performed utilizing CrO₃ in the presence of an acid (e.g., H₂SO₄) in a solvent (e.g., acetone).

In some embodiments, the present disclosure provides a method comprising: reducing a compound, wherein the compound comprises a 3-ketone group bonded to moiety A (e.g., a compound of formula I wherein R¹ and R^{1a} are taken together to form ═O) or a salt thereof; and producing a compound comprises two 3-H boned to moiety A (e.g., a compound of formula I wherein R¹ and R^{1a} are —H) or a salt thereof.

In some embodiments, the two compounds are otherwise identical except that prior to reduction, R¹ and R^{1a}are taken together to form ═O, while after reduction, both R¹ and R^{1a} are —H. Various reducing technologies are available to those skilled in the art and can be utilized in accordance with the present disclosure, for example, in some embodiments, a reduction is performed utilizing hydrazine in the presence of a base (e.g., KOH) in a polar solvent (e.g., a protic solvent, an alcohol, etc.). In some embodiments, a solvent is 2, 2′-oxybis(ethan-1-ol).

In some embodiments, the present disclosure provides a method comprising:
oxidizing a 7-OH group bonded to moiety A in a compound (e.g., a compound of formula I wherein one of R³ or R^{3a} is —OH and the other is —H) or a salt thereof, and
producing a compound comprising a 7-ketone group bonded to moiety A (e.g., a compound of formula I wherein R³ and R^{3a} are taken together to form ═O) or a salt thereof.
   In some embodiments, the two compounds are otherwise identical except that prior to oxidation, one of R³ and R^{3a} is —OH and the other is —H (e.g., R³ is —OH and R^{3a} is —H), while after oxidation, R³ and R^{3a} are taken together to form ═O. Various oxidizing technologies are available to those skilled in the art and can be utilized in accordance with the present disclosure, for example, in some embodiments, an oxidation is performed utilizing PCC in a solvent (e.g., DCM).

In some embodiments, the present disclosure provides a method comprising:
contacting a compound comprising a ketone group, e.g., 7-ketone group, bonded to moiety A (e.g., a compound of formula I wherein R³ and R^{3a} are taken together to form ═O) or a salt thereof with a compound having the structure of H₂NR or a salt thereof, and
producing a compound comprising a ═NR^{x}, e.g., 7-(═NR^{x}) group, bonded to moiety A (e.g., a compound of formula I wherein R³ and R^{3a} are taken together to form ═NR^{x}) or a salt thereof.
   In some embodiments, the two compounds are otherwise identical except that prior to performing the method, R³ and R^{3a} are taken together to form ═O while after performing the method they are taken together to form ═NR. Various technologies are available to those skilled in the art and can be utilized in accordance with the present disclosure to perform a method, for example, in some embodiments, a salt of H₂NR is utilized (e.g., a HCl salt) in the presence of or in a base (e.g., pyridine). In some embodiments, as described herein, a reaction may be performed at a temperature higher than room temperature (e.g., at 100° C.).

In some embodiments, the present disclosure provides a method comprising:
contacting a compound comprising a leaving group bonded to moiety A (e.g., bonded to carbon 17 such as in a compound of formula I wherein R¹ is a leaving group such as —Cl, —Br or —I) or a salt thereof with a base; and
producing a compound comprising a double bond within moiety A (e.g., between carbon 16 and carbon 17 such as in a compound of formula I wherein R⁵ and R⁶ or R^{6a} are taken together to form a bond) or a salt thereof.
   In some embodiments, the two compounds are otherwise identical except that prior to performing the method, R is a leaving group, e.g., —Cl, and one of R⁶ and R^{6a} is —H while after performing the method R⁵ and R⁶ or R^{6a} are taken together to form a double bond. Various technologies are available to those skilled in the art and can be utilized in accordance with the present disclosure to perform a method, for example, in some embodiments, a base is pyridine; in some embodiments, a leaving group is —Cl. In some embodiments, as described herein, a reaction may be performed at a temperature higher than room temperature (e.g., in refluxing pyridine).

In some embodiments, the present disclosure provides a method comprising:
oxidizing a compound comprising two —H bonded to carbon 15 of moiety A (e.g., a compound of formula I wherein R⁷ and R^{7a} are —H) or a salt thereof, and
producing a compound comprising a 15-ketone group bonded to moiety A (e.g., a compound of formula I wherein R⁷ and R^{7a} are taken together to form ═O) or a salt thereof.
   In some embodiments, the two compounds are otherwise identical except that prior to oxidation, R⁷ and R^{7a} are —H, while after oxidation, R⁷ and R^{7a} are taken together to form ═O. In some embodiments, R⁵ and R⁶ or R^{6a} are taken together to form a double bond in the compounds both before and after oxidation. Various oxidizing technologies are available to those skilled in the art and can be utilized in accordance with the present disclosure, for example, in some embodiments, an oxidation is performed utilizing a reagent system comprising CrO₃ in the presence of 3,5-dimethyl-1H-pyrazole in a solvent (e.g., DCM).

In some embodiments, the present disclosure provides a method comprising: reducing a compound comprising a 15-ketone group bonded to moiety A (e.g., a compound of formula I wherein R⁷ and R^{7a} are taken together to form ═O) or a salt thereof; and producing a compound comprising a 15-hydroxy group bonded to moiety A (e.g., a compound of formula I wherein one of R⁷ and R^{7a} is —H and the other is —OH) or a salt thereof. In some embodiments, the two compounds are otherwise identical except that prior to reduction, R⁷ and R^{7a} are taken together to form ═O, while after reduction, one of R⁷ and R^{7a} is —OH and the other is —H. In some embodiments, R⁷ is —H. In some embodiments, R^{7a} is —H. In some embodiments, R⁵ and R⁶ or R^{6a} re taken together to form a double bond in the compounds both before and after reduction. Various reducing technologies are available to those skilled in the art and can be utilized in accordance with the present disclosure, for example, in some embodiments, a reduction is performed utilizing a reagent system comprising NaBH₄ in the presence of CeCl₃ in a solvent (e.g., a mixture of THF and MeOH). In some embodiments, a reaction is performed at a temperature below room temperature (e.g., at 0° C.).

In some embodiments, the present disclosure provides a method comprising:
oxidizing a compound comprising a —CHO group and moiety A (e.g., a compound of formula I wherein R^{L} is —CHO) or a salt thereof, and
producing a compound comprising a —COOH group and moiety A (e.g., a compound of formula I wherein R^{L} is —COOH) or a salt thereof.
   In some embodiments, the two compounds are otherwise identical except that prior to oxidation, R^{L} is —CHO, while after oxidation, R^{L} is —COOH. Various oxidizing technologies are available to those skilled in the art and can be utilized in accordance with the present disclosure, for example, in some embodiments, an oxidation is performed utilizing a reagent system comprising NaClO₂ in the presence of a suitable salt (e.g., a buffer can provide pH buffering such as NaH₂PO₄) a solvent (e.g., a protic solvent such as a mixture of t-BuOH and H₂O).

In some embodiments, the present disclosure provides a method comprising:
contacting a compound comprising a —COOH group and moiety A (e.g., a compound of formula I wherein R^{L} is —COOH) or a salt thereof with a compound having the structure of NH(R′)S(O)₂R^{s} or a salt thereof, and
producing a compound comprising a —C(O)N(R′)S(O)₂R^{s} group and moiety A (e.g., a compound of formula I wherein R^{L} is —C(O)N(R′)S(O)₂R^{s}) or a salt thereof.
   In some embodiments, the two compounds are otherwise identical except that prior to performing the method, R^{L} is —COOH or a salt form thereof, while after performing the method, R^{L} is —C(O)N(R′)S(O)₂R^{s} or a salt form thereof. Various technologies are available to those skilled in the art and can be utilized in accordance with the present disclosure to perform a method, for example, in some embodiments, contacting is performed in the presence of condensing reagent (e.g., EDCI) and/or a catalyst or base (e.g., DMAP) in a solvent (e.g., DCM). In some embodiments, a reaction is performed at a temperature below room temperature (e.g., at about 0° C.). In some embodiments, temperature changes during a reaction process, e.g., from about 0° C. to room temperature.

In some embodiments, R′ is —H or optionally substituted C₁₋₆ aliphatic. In some embodiments, R′ is —H. In some embodiments, R′ is C₁₋₆ aliphatic. In some embodiments, R′ is C₁₋₆ alkyl. In some embodiments, —N(R′)— is —NH—.

In some embodiments, R^{s} is -L″-R′ wherein each of L″ and R′ is independently as described herein. In some embodiments, R^{s} is R′ as described herein. In some embodiments, R′ is —H. In some embodiments, R′ is optionally substituted C₁₋₆ aliphatic. In some embodiments, R′ is t-butyl. In some embodiments, R′ is methyl. In some embodiments, R′ is ethyl.

In some embodiments, a compound having the structure of NH(R′)S(O)₂R^{s} is NH₂S(O)₂R^{s}. In some embodiments, a compound having the structure of NH(R′)S(O)₂R^{s} is NH₂S(O)₂Me. In some embodiments, it is NH₂S(O)₂Et. In some embodiments, it is NH₂S(O)₂t-Bu.

In some embodiments, R^{L} is —C(O)NHS(O)₂R^{s}. In some embodiments, R^{s} is —H. In some embodiments, R^{s} is C₁₋₆ aliphatic. In some embodiments, R^{s} is C₁₋₆ alkyl. In some embodiments, R^{s} is methyl. In some embodiments, R^{s} is ethyl. In some embodiments, R^{s} is t-butyl.

In some embodiments, the present disclosure provides a method comprising:
contacting a compound comprising a —COOH group and moiety A (e.g., a compound of formula I wherein R^{L} is —COOH) or a salt thereof with an azide agent (a compound which comprise and/or provide —N₃) or a salt thereof; and
producing a compound comprising a —NCO group and moiety A (e.g., a compound of formula I wherein R^{L} is —NCO) or a salt thereof.
   In some embodiments, the two compounds are otherwise identical except that prior to performing the method, R^{L} is —COOH or a salt form thereof, while after performing the method, R^{L} is —NCO or a salt form thereof. Various technologies are available to those skilled in the art and can be utilized in accordance with the present disclosure to perform a method, for example, in some embodiments, contacting is performed in the presence of an azide source (e.g., DPPA) and/or a base (e.g., K₂CO₃) in a solvent (e.g., dioxane or R^{s}—OH). In some embodiments, a reaction is performed at a temperature above room temperature (e.g., at about 80° C., in refluxing R^{s}—OH, etc.). In some embodiments, a reaction is performed at a temperature below room temperature (e.g., at about 0° C.). In some embodiments, temperature changes during a reaction process, e.g., from about 0° C. to room temperature. In some embodiments, a compound comprising a —NCO group and moiety A (e.g., a compound of formula I wherein R^{L} is —NCO) or a salt thereof is utilized in another reaction without isolation. Those skilled in the art will appreciate if such a compound is generated as a product or intermediate, e.g., based on organic chemistry principles, reactions, mechanisms, etc. In some embodiments, —NCO is reacted with a nucleophile, e.g., compounds comprising nucleophilic nitrogen, oxygen or sulfur, etc., to provide various compounds, e.g., compounds comprising —NHC(O)O—, —NHC(O)S—, NHC(O)N(R′)—, etc. Various suitable nucleophiles are available to those skilled in the art and can be utilized in accordance with the present disclosure. For example, in some embodiments, a nucleophile is R^{s}—OH or a salt thereof wherein R^{s} is as described herein. In some embodiments, a nucleophile is NH(R′)S(O)₂R^{s} or a salt thereof, wherein each variable is independently as described herein. In some embodiments, a nucleophile is NH₂S(O)₂R^{s} or a salt thereof wherein R^{s} is as described herein.

In some embodiments, the present disclosure provides a method comprising:
contacting a compound comprising a —COOH group and moiety A (e.g., a compound of formula I wherein R^{L} is —COOH) or a salt thereof with a compound having the structure of NH(R′)S(O)₂R^{s} (e.g., NH₂S(O)₂R^{s}) or a salt thereof, and
producing a compound comprising a —N(R′)C(O)N(R′)S(O)₂R^{s} group and moiety A (e.g., a compound of formula I wherein R^{L} is —N(R′)C(O)N(R′)S(O)₂R^{s}) or a salt thereof.
   In some embodiments, the two compounds are otherwise identical except that prior to performing the method, R^{L} is —COOH or a salt form thereof, while after performing the method, R^{L} is —N(R′)C(O)N(R′)S(O)₂R^{s} or a salt form thereof, wherein each variable is independently as described herein. In some embodiments, —N(R′)C(O)N(R′)S(O)₂R^{s} is —NHC(O)N(R′)S(O)₂R^{s}, wherein each variable is independently as described herein. Various technologies are available to those skilled in the art and can be utilized in accordance with the present disclosure to perform a method, for example, in some embodiments, contacting is performed in the presence of an azide source (e.g., DPPA) and/or a base (e.g., K₂CO₃) in a solvent (e.g., dioxane). In some embodiments, a reaction is performed at a temperature below room temperature (e.g., at about 0° C.). In some embodiments, temperature changes during a reaction process, e.g., from about 0° C. to room temperature.

In some embodiments, the present disclosure provides a method comprising:
contacting a compound comprising a —COOH group and moiety A (e.g., a compound of formula I wherein R^{L} is —COOH) or a salt thereof with a compound having the structure of R^{s}—OH (e.g., wherein R^{s} is optionally substituted C₁₋₆ aliphatic) or a salt thereof, and
producing a compound comprising a —N(R′)C(O)OR^{s} group and moiety A (e.g., a compound of formula I wherein R^{L} is —N(R′)C(O)OR^{s}) or a salt thereof.
   In some embodiments, the two compounds are otherwise identical except that prior to performing the method, R^{L} is —COOH or a salt form thereof, while after performing the method, R^{L} is —N(R′)C(O)OR^{s} or a salt form thereof, wherein each variable is independently as described herein. In some embodiments, —N(R′)C(O)OR^{s} is —NHC(O)OR^{s}, wherein each variable is independently as described herein. Various technologies are available to those skilled in the art and can be utilized in accordance with the present disclosure to perform a method, for example, in some embodiments, contacting is performed in the presence of an azide source (e.g., DPPA) and/or a base (e.g., K₂CO₃) in a solvent (e.g., dioxane or R^{s}—OH). In some embodiments, a reaction is performed at a temperature above room temperature (e.g., at about 80° C., in refluxing R^{s}—OH, etc.). In some embodiments, R^{s} is R′ as described herein. In some embodiments, R^{s} is R as described herein. In some embodiments, R^{s} is optionally substituted C₁₋₆ aliphatic, e.g., t-Bu. In some embodiments, —N(R′)C(O)OR^{s} is a protected amino group. In some embodiments, a provided method comprises de-protecting a compound comprising a protected amino group, e.g., a compound of formula I comprising a protected amino group, e.g., —N(R′)C(O)OR^{s}, or a salt thereof, to provide a compound comprising a de-protected amino group, e.g., a compound of formula I comprising —NH(R′) or a salt thereof. In some embodiments, a provided method comprises de-protecting a compound of formula I wherein R^{L} is —N(R′)C(O)OR^{s} or a salt thereof to provide a compound of formula I wherein R^{L} is —NH(R′) or a salt thereof. In some embodiments, before deprotection R^{L} is —N(R′)C(O)OR^{s} and after deprotection R^{L} is —NH₂. In some embodiments, R^{s} is optionally substituted C₁₋₆ aliphatic. In some embodiments, R^{s} is t-butyl.

In some embodiments, the present disclosure provides a method comprising:
contacting a compound comprising an amino group (e.g., —NHR wherein R is as described herein) and moiety A (e.g., a compound of formula I wherein R^{L} is —NHR wherein R is as described herein) or a salt thereof with an electrophile, wherein the amino group reacts with the electrophile.
   In some embodiments, a compound is a compound of formula I wherein R^{L} is —NH₂ or a salt thereof. Suitable electrophiles are widely known in the art and can be utilized in accordance with the present disclosure. In some embodiments, an electrophile is CS₂. In some embodiments, a method comprises contacting CS₂ in the presence of a base (e.g., NR₃ such as Et₃N) in a suitable solvent (e.g., anhydrous ethanol) at room temperature. In some embodiments, a product formed after contact with an electrophile, e.g., CS₂, is further reacted other reagents. For example, in some embodiments, a product formed with CS₂ is contacted with Boc₂O in the presence of DMAP in a suitable solvent (e.g., EtOH) to form a product comprising —NCS. In some embodiments, a formed product comprises a —NCS group and moiety A. In some embodiments, a formed product is a compound of formula I wherein R^{L} is —NCS or a salt thereof. In some embodiments, a reaction comprises a stage, e.g., at the beginning, at a temperature lower than room temperature (e.g., −5° C.) and warming up to a higher temperature, e.g., room temperature.

In some embodiments, the present disclosure provides a method comprising:
contacting a compound comprising a —NCS and moiety A (e.g., a compound of formula I wherein R^{L} is —NCS) or a salt thereof with an electrophile.
   Many electrophiles are available and can be utilized in accordance with the present disclosure. In some embodiments, an electrophile is a nitrogen electrophile. In some embodiments, an electrophile is NH(R′)₂ wherein each R′ is independently as described herein. In some embodiments, an electrophile is NH₂R′ wherein R′ is as described herein. In some embodiments, an electrophile is compound having the structure of NH(R′)S(O)₂R^{s} (e.g., NH₂S(O)₂R^{s}) or a salt thereof. In some embodiments, the present disclosure provides a method comprising:
contacting a compound comprising a —NCS and moiety A (e.g., a compound of formula I wherein R^{L} is —NCS) or a salt thereof with a compound having the structure of NH(R′)S(O)₂R^{s} (e.g., NH₂S(O)₂R^{s}) or a salt thereof, and
producing a compound comprising a —N(R′)C(S)N(R′)S(O)₂R^{s} group and moiety A (e.g., a compound of formula I wherein R^{L} is —N(R′)C(S)N(R′)S(O)₂R^{s}) or a salt thereof.
   In some embodiments, the two compounds are otherwise identical except that prior to performing the method, R^{L} is —NCS or a salt form thereof, while after performing the method, R^{L} is —N(R′)C(S)N(R′)S(O)₂R^{s} or a salt form thereof, wherein each variable is independently as described herein. In some embodiments, —N(R′)C(S)N(R′)S(O)₂R^{s} is —NHC(S)N(R′)S(O)₂R^{s}, wherein each variable is independently as described herein. Various technologies are available to those skilled in the art and can be utilized in accordance with the present disclosure to perform a method, for example, in some embodiments, contacting is performed in the presence of a base (e.g., K₂CO₃, DBU, etc.) in a solvent (e.g., acetone, THF, toluene, etc.). In some embodiments, a reaction is performed at a temperature above room temperature (e.g., a refluxing solvent such as acetone).

In some embodiments, the present disclosure provides a method comprising:
contacting a compound comprising an amino group and moiety A (e.g., a compound of formula I wherein R^{L} is —NHR wherein R is optionally substituted (e.g., —H, optionally substituted C₁₋₆ aliphatic, etc.; in some embodiments, —NHR is —NH₂) or a salt thereof with a compound having the structure of NH(R′)S(O)₂R^{s} (e.g., NH₂S(O)₂R^{s}) or a salt thereof and oxalyl chloride; and
producing a compound comprising a —N(R′)C(O)C(O)N(R′)S(O)₂R^{s} group and moiety A (e.g., a compound of formula I wherein R^{L} is —N(R′)C(O)C(O)N(R′)S(O)₂R^{s}) or a salt thereof.
   In some embodiments, the two compounds are otherwise identical except that prior to performing the method, R^{L} is —NH₂ or a salt form thereof, while after performing the method, R^{L} is —N(R′)C(O)C(O)N(R′)S(O)₂R^{s} or a salt form thereof, wherein each variable is independently as described herein. In some embodiments, —N(R′)C(O)C(O)N(R′)S(O)₂R^{s} is —NHC(O)C(O)N(R′)S(O)₂R^{s}, wherein each variable is independently as described herein. Various technologies are available to those skilled in the art and can be utilized in accordance with the present disclosure to perform a method, for example, in some embodiments, contacting is performed in the presence of a base (e.g., N(R)₃ such as TEA) in a solvent (e.g., DCM). In some embodiments, a reaction is performed at a temperature below room temperature (e.g., at about 0° C.). In some embodiments, temperature changes during a reaction process, e.g., from about 0° C. to room temperature.

In some embodiments, the present disclosure provides a method comprising:
contacting a compound comprising a ketone group and moiety A (e.g., a compound of formula I wherein R¹ and R^{1a} are taken together to form ═O) or a salt thereof with fluorination agent; and
producing a compound comprising two —F and moiety A (e.g., a compound of formula I wherein R¹ and R^{1a} are —F) or a salt thereof.
   In some embodiments, the two compounds are otherwise identical except that prior to performing the method, R¹ and R^{1a} are taken together to form ═O, while after performing the method, R¹ and R^{1a} are —F. In some embodiments, compounds are protected for the reaction, e.g., —OH protected as —OC(O)R (e.g., —OAc), —C(O)OH protected as —C(O)OR wherein R is not —H (e.g., —C(O)OMe), etc. Various technologies are available to those skilled in the art and can be utilized in accordance with the present disclosure to perform a method, for example, in some embodiments, a fluorination agent is or comprises DAST or a salt thereof. In some embodiments, a reaction is performed at a temperature above room temperature (e.g., at about 60° C.). In some embodiments, a reaction is performed in a suitable solvent such as toluene. In some embodiments, a provided method further comprises de-protection of protected groups, e.g., protected —OH, protected —C(O)OH, etc. For example, in some embodiments, as described in the Examples de-protection is performed in refluxing methanol in the presence of a base such as NaOH. Various protecting and de-protecting technologies are available and can be utilized in accordance with the present disclosure.

In some embodiments, the present disclosure provides method for reducing a compound. For example, in some embodiments, the present disclosure provides a method comprising:
reducing a compound comprising a ketone group and moiety A (e.g., a compound comprising a 3-ketone group bonded to moiety A, a compound of formula I wherein R¹ and R^{1a} are taken together to form ═O) or a salt thereof, and
producing a compound comprising a hydroxyl group and moiety A (e.g., a compound comprising 3-OH and 3-H bonded to moiety A, a compound of formula I wherein one of R¹ and R^{1a} is —H and the other is —OH, etc.) or a salt thereof.
   In some embodiments, the two compounds are otherwise identical except that prior to performing the method, R¹ and R^{1a} are taken together to form ═O, while after performing the method, one of R¹ and R^{1a} is —H and the other is —OH. In some embodiments, a reduction product contains enriched D. In some embodiments, R¹ or R^{1a} is D. In some embodiments, R¹ or R^{1a} is hydrogen and is enriched for D. Various reducing technologies are available to those skilled in the art and can be utilized in accordance with the present disclosure. In some embodiments, a reducing agent is enriched for D. In some embodiments, a reducing agent comprises or is enriched for D. In some embodiments, a reducing agent is LiAlD₄. In some embodiments, certain groups of a compound are each independently protected. In some embodiments, R^{L} is —C(O)R^{s} wherein R^{s} is as described herein. In some embodiments, R^{L} is —C(O)OR wherein R is as described herein and is not —H. In some embodiments, R^{L} is —C(O)OR wherein R is optionally substituted C₁₋₆ aliphatic. In some embodiments, R^{L} is —C(O)OMe. In some embodiments, a reduction is performed in a suitable solvent, e.g., Et₂O. In some embodiments, a reaction is performed at a temperature below room temperature (e.g., at about 0° C.). In some embodiments, temperature changes during a reaction process, e.g., from about 0° C. to room temperature.

In some embodiments, the present disclosure provides a method comprising converting a hydroxyl group into a leaving group. In some embodiments, the present disclosure provides a method comprising:
reacting a compound comprising a hydroxyl group and moiety A (e.g., a compound comprising a 3-OH group bonded to moiety A, a compound of formula I wherein one of R¹ and R^{1a} is —H and the other is —OH, etc.) or a salt thereof; and
producing a compound comprising a leaving group and moiety A (e.g., a compound comprising 3-leaving group and 3-H bonded to moiety A, a compound of formula I wherein one of R¹ and R^{1a} is —H and the other is a leaving group, etc.) or a salt thereof.
   In some embodiments, the two compounds are otherwise identical except that prior to performing the method, one of R¹ and R^{1a} is —H and the other is —OH, while after performing the method, one of R¹ and R^{1a} is —H and the other is leaving group. Various leaving group can be utilized in accordance with the present disclosure. In some embodiments, a leaving group is —OS(O)₂R^{s} wherein R^{s} is as described herein. In some embodiments, in a produced compound one of R¹ or R^{1a} is —OS(O)₂R^{s} and the other is —H (in some embodiments, is D or is enriched for D). In some embodiments, R^{s} is optionally substituted C₁₋₆ aliphatic. In some embodiments, R^{s} is optionally substituted phenyl. In some embodiments, R^{s} is methyl. In some embodiments, reacting is performed in the presence of a sulfonylation reagent, e.g., a compound having the structure of Cl—OS(O)₂R^{s} or a salt thereof and a base, e.g., N(R)₃ wherein each R is independently as described herein (e.g., —H or C₁₋₆ aliphatic) such as Et₃N. In some embodiments, a reaction is performed in a suitable solvent, e.g., DCM. In some embodiments, a reaction is performed at a temperature below room temperature (e.g., at about 0° C.). In some embodiments, temperature changes during a reaction process, e.g., from about 0° C. to room temperature. In some embodiments, various groups of a compound is independently protected. In some embodiments, R^{L} is —C(O)R^{s} wherein R^{s} is as described herein. In some embodiments, R^{L} is —C(O)OR wherein R is as described herein and is not —H. In some embodiments, R^{L} is —C(O)OR wherein R is optionally substituted C₁₋₆ aliphatic. In some embodiments, R^{L} is —C(O)OMe. In some embodiments, a reduction is performed in a suitable solvent, e.g., Et₂O. In some embodiments, a reaction is performed at a temperature below room temperature (e.g., at about 0° C.). In some embodiments, temperature changes during a reaction process, e.g., from about 0° C. to room temperature.

In some embodiments, the present disclosure provides a method comprising converting a leaving group to —H which in some embodiments is D. In some embodiments, the present disclosure provides a method comprising:
contacting a compound comprising a leaving group (e.g., —OS(O)₂R^{s} such as —OMs) and moiety A (e.g., a compound comprising 3-leaving group and 3-H bonded to moiety A, a compound of formula I wherein one of R¹ and R^{1a} is —H and the other is a leaving group, etc.) or a salt thereof with a reducing agent; and
producing a compound moiety A but not the leaving group (e.g., a compound comprising two 3-H bonded to moiety A, a compound of formula I wherein both R¹ and R^{1a} are —H, etc.) or a salt thereof.
   In some embodiments, the two compounds are otherwise identical except that prior to performing the method, one of R¹ and R^{1a} is —H and the other is a leaving group, while after performing the method, both R¹ and R^{1a} are —H. In some embodiments, each of R¹ and R^{1a} is independently and optionally D or enriched for D. In some embodiments, one of R¹ and R^{1a} is D and the other is not D. In some embodiments, one of them is enriched for D and the other is not. In some embodiments, both R¹ and R^{1a} are independently D. In some embodiments, both of them are independently enriched for D. In some embodiments, a leaving group is —OS(O)₂R^{s} wherein R^{s} is as described herein. In some embodiments, R^{s} is optionally substituted C₁₋₆ aliphatic. In some embodiments, R^{s} is optionally substituted phenyl. In some embodiments, R^{s} is methyl. Various reducing technologies are available to those skilled in the art and can be utilized in accordance with the present disclosure. In some embodiments, a reducing agent is LiAlH₄. In some embodiments, a reducing agent is enriched for D. In some embodiments, a reducing agent comprises or is enriched for D. In some embodiments, a reducing agent is LiAlH₄. In some embodiments, a reducing agent is LiAlD₄. In some embodiments, certain groups of a compound are each independently protected. In some embodiments, R^{L} is —C(O)R^{s} wherein R^{s} is as described herein. In some embodiments, R^{L} is —C(O)OR wherein R is as described herein and is not —H. In some embodiments, R^{L} is —C(O)OR wherein R is optionally substituted C₁₋₆ aliphatic. In some embodiments, R^{L} is —C(O)OMe. In some embodiments, a reduction is performed in a suitable solvent, e.g., Et₂O. In some embodiments, a reaction is performed at a temperature below room temperature (e.g., at about 0° C.). In some embodiments, temperature changes during a reaction process, e.g., from about 0° C. to room temperature. In some embodiments, a compound is

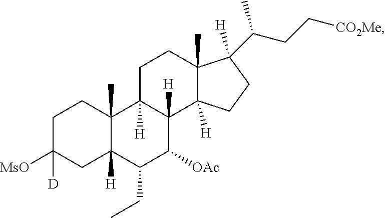

and a product is

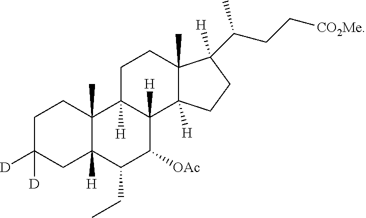

In some embodiments, a method comprises de-protecting a protected carboxyl group. In some embodiments, a method comprises converting a compound comprising —C(O)OR wherein R is not —H and moiety A (e.g., a compound of formula I wherein R^{L} is —C(O)OR wherein R is not —H) to provide a compound comprising —C(O)OH and moiety A (e.g., a compound of formula I wherein R^{L} is —C(O)OH) or a salt thereof. In some embodiments, R is optionally substituted C₁₋₆ aliphatic. Various suitable protecting/de-protection technologies are widely known and can be utilized in accordance with the present disclosure. In some embodiments, R is methyl. In some embodiments, de-protecting is in the presence of a base, e.g., NaOH, in a suitable solvent (e.g., EtOH/H₂O). In some embodiments, a reaction comprises a stage performed at a temperature higher than room temperature, e.g., in a refluxing solvent such as EtOH.

In some embodiments, the present disclosure provides a method comprising:
contacting a compound comprising a ketone group and moiety A (e.g., a compound of formula I wherein R¹ and R^{1a} are taken together to form ═O) or a salt thereof with fluorination agent; and
producing a compound comprising two —F and moiety A (e.g., a compound of formula I wherein R¹ and R^{1a} are —F) or a salt thereof.
   In some embodiments, the two compounds are otherwise identical except that prior to performing the method, R¹ and R^{1a} are taken together to form ═O, while after performing the method, R¹ and R^{1a} are —F. In some embodiments, certain groups are protected for the reaction, e.g., —OH protected as —OC(O)R (e.g., —OAc), —C(O)OH protected as —C(O)OR wherein R is not —H (e.g., —C(O)OMe), etc. Varioustechnologies are available to those skilled in the art and can be utilized in accordance with the present disclosure to perform a method, for example, in some embodiments, a fluorination agent is or comprises DAST or a salt thereof. In some embodiments, a reaction is performed at a temperature above room temperature (e.g., at about 60° C.). In some embodiments, a reaction is performed in a suitable solvent such as toluene. In some embodiments, a provided method further comprises de-protection of protected groups, e.g., protected —OH, protected —C(O)OH, etc. For example, in some embodiments, as described in the Examples de-protection is performed in refluxing methanol in the presence of a base such as NaOH. Various protecting and de-protecting technologies are available and can be utilized in accordance with the present disclosure.

Those skilled in the art appreciates that various compounds of present disclosure, e.g., those with reactive groups such as amino groups (e.g., wherein R^{L} is —NH₂), carboxyl groups (e.g., wherein R^{L} is —COOH), etc. may be utilized to prepare many types of compounds. For example, in some embodiments, an amino group may be converted to —N(R^{s})₂, —N(R′)C(O)N(R′)S(O)₂R^{s}, —N(R′)C(S)R^{s}, —N(R′)C(S)N(R′)S(O)₂R′, —N(R′)C(O)C(O)N(R′)S(O)₂R′, —N(R′)C(O)N(R′)S(O)₂N(R′)₂, —N(R′)C(O)R^{s}, —N(R′)C(O)OR^{s}, —N(R′)C(O)N(R^{s})₂, or —N(R′)S(O)₂R^{s}, etc. In some embodiments, —COOH or a salt form thereof may be converted into R^{s}, —C(O)OR^{s}, —C(O)N(R^{s})₂, —C(O)N(R′)S(O)₂R^{s}, —C(O)N(R′)C(R′)₂C(O)N(R′)₂, —C(O)N(R′)C(R′)₂S(O)₂R′, —C(O)N(R′)C(R′)₂S(O)₂N(R′)₂, —C(O)N(R′)C(R′)₂P(O)(R′)₂, —C(O)N(R′)C(R′)₂N(R′)C(O)N(R′)S(O)₂R′, —C(O)N(R′)C(R′)₃, —C(O)N(R′)C(R′)₂C(O)N(R′)S(O)₂R′, —C(O)N(R′)S(O)₂N(R′)₂, —C(O)N(R′)C(NR′)N(R′)₂, —N(R′)C(O)N(R′)S(O)₂R′, —N(R′)C(S)R′, —N(R′)C(S)N(R′)S(O)₂R′, —N(R′)C(O)C(O)N(R′)S(O)₂R′, —N(R′)C(O)N(R′)S(O)₂N(R^{s})₂, —N(R′)C(O)R^{s}, —N(R′)C(O)OR^{s}, —N(R′)C(O)N(R^{s})₂, etc. Many technologies are available for converting a first group (e.g., an amino group, a carboxyl group, etc.) into a different second group. Certain first and second groups and useful transformation technologies are described in the Examples.

For example, in some embodiments, —COOH can be converted into an amide. In some embodiments, the present disclosure provides a method comprising:
contacting a compound comprising a carboxyl group and moiety A (e.g., a compound of formula I wherein R^{L} is —COOH) or a salt thereof (or an activated form thereof) with an agent having the structure of NH(R^{s})₂ or a salt thereof wherein each R^{s} is independently as described herein; and
producing a compound comprising —C(O)N(R^{s})₂ and moiety A (e.g., a compound of formula I wherein R^{L} is —C(O)N(R^{s})₂) or a salt thereof.
   In some embodiments, NH(R^{s})₂ is NH₂R^{s} wherein R^{s} is as described herein. In some embodiments, R^{s} is R as described herein. For example, in some embodiments, R is optionally substituted 5-membered heteroaryl having 1-4 (e.g., 1, 2, 3 or 4) heteroatoms each independently selected from nitrogen, oxygen and sulfur. In some embodiments, R is optionally substituted tetrazolyl. In some embodiments, R is tetrazolyl. Various technologies, e.g., various types of amidation technologies, are available to those skilled in the art and can be utilized in accordance with the present disclosure. In some embodiments, contacting is performed in the presence of a coupling agent (e.g., EDCI) and/or a base or catalyst (e.g., DMAP) in a suitable solvent. In some embodiments, a solvent comprises DCM and DMF. In some embodiments, a reaction is performed at a temperature higher that room temperature (e.g., at about 60° C.).

In some embodiments, —COOH can be converted to R^{s} wherein R^{s} is -L″-R′, wherein each L″ and R′ are independently as described herein. In some embodiments, L″ is -L wherein L^{a}″ is an optionally substituted 5-membered heteroaryl ring having 3 heteroatoms two of which are neighboring nitrogen. In some embodiments, the present disclosure provides a method comprising:
contacting a compound comprising a carboxyl group and moiety A (e.g., a compound of formula I wherein R^{L} is —COOH) or a salt thereof (or an activated form thereof) with a hydrazide agent having the structure of NH₂NHC(O)R^{s} or a salt thereof and a thiation agent; and
producing a compound comprising

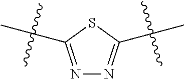 [SMILES: *c1nnc(*)s1]

and moiety A (e.g., a compound of formula I wherein R^{L} is

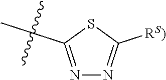

or a salt thereof.
In some embodiments, the two compounds are otherwise identical except that prior to performing the method, R^{L} is —COOH or a salt form thereof while after performing the method in a produced compound R^{L} is

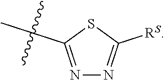

In some embodiments, R^{s} is -L″-R′ wherein each of L″ and R′ is independently as described herein. In some embodiments, L″ is a covalent bond. In some embodiments, a hydrazide agent has the structure of NH₂NHC(O)R′ or a salt thereof wherein R′ is as described herein. In some embodiments, R^{L} is

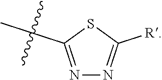

In some embodiments, R′ is R as described herein. In some embodiments, R is optionally substituted C₁₋₆ aliphatic. In some embodiments, R is isopropyl. Various technologies are available to those skilled in the art and can be utilized in accordance with the present disclosure to perform a method. In some embodiments, contacting is conducted in the presence of or comprises contact of a starting material an intermediate with a coupling agent/water scavenger agent and/or abase (e.g., N(R)₃ wherein each R is independently —H or optionally substituted C₁₋₆ aliphatic such as Et₃N). In some embodiments, a thiolation agent is Lawesson's reagent. In some embodiments, a coupling/water scavenger agent is T₃P. In some embodiments, a reaction is performed in a suitable solvent, e.g., a solvent which is or comprises EtOAc. In some embodiments, a reaction is performed at a temperature above room temperature (e.g., at about 80° C.). In some embodiments, certain groups are protected for the reaction, e.g., —OH protected as —OSi(R)₃ wherein each R is independently optionally substituted C₁₋₆ aliphatic or phenyl (e.g., —OTMS), —OMs, etc. Various protecting and de-protecting technologies are available and can be utilized in accordance with the present disclosure. For example, as shown in the Examples a hydroxyl group may be protected by contacting it with TMSCI, NMI and BSA and can be deprotected by contacting the protected group with an acid, e.g., HCl.

In some embodiments, as appreciated by those skilled in the art, a —COOH group such as R^{L}may be activated/derivatized for further reactions and/or transformations. For example, in some embodiments, the present disclosure provides a method comprising:
contacting a compound comprising a carboxyl group and moiety A (e.g., a compound of formula I wherein R^{L} is —COOH) or a salt thereof to provide a —C(O)OC(O)— and moiety A (e.g., a compound of formula I wherein R^{L} is —C(O)OR^{s} wherein R^{s} is —C(O)R′) or a salt thereof.
   In some embodiments, the two compounds are otherwise identical except that prior to performing the method, R^{L} is —COOH or a salt form thereof while after performing the method in a produced compound R^{L} is —C(O)OR^{s} wherein R^{s} is —C(O)R′ wherein R′ is as described herein. In some embodiments, R′ is R as described herein. In some embodiments, R′ is —OR wherein R is as described herein and is not —H. In some embodiments, R is optionally substituted C₁₋₆ aliphatic. In some embodiments, R is isopropyl. In some embodiments, in a produced compound R^{L} is —C(O)OC(O)OR wherein R is as described herein, e.g., isopropyl. Various technologies are available to those skilled in the art and can be utilized in accordance with the present disclosure to perform a method. In some embodiments, contacting is conducted in the presence of an acylating agent and/or a base (e.g., N(R)₃ wherein each R is independently —H or optionally substituted C₁₋₆ aliphatic such as Et₃N). In some embodiments, an acylating agent is a compound has the structure of LG-C(O)R′ or a salt thereof, wherein LG is a leaving group and R′ is as described herein. In some embodiments, LG is —Cl. In some embodiments, an acylating agent is ClCO₂i-Pr. In some embodiments, a reaction is performed in a suitable solvent, e.g., a solvent which is or comprises THF. In some embodiments, a reaction is performed at a temperature below room temperature (e.g., at about 0° C.). In some embodiments, a reaction comprises a change of temperature, e.g., from a lower temperature (e.g., about 0° C.) to a higher temperature (e.g., about room temperature (for the purpose of the present disclosure 298.15K unless specified otherwise)).

In some embodiments, the present disclosure provides a method comprising:
contacting a compound comprising a carboxyl group and moiety A (e.g., a compound of formula I wherein R^{L} is —COOH or an activated form thereof) or a salt thereof or an activated form thereof (e.g., anhydride, acyl chloride, etc.) with a hydrazinecarboximidamide agent; and
producing a compound comprising optionally substituted

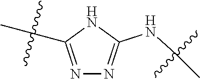 [SMILES: *Nc1nnc(*)[nH]1]

and moiety A (e.g., a compound of formula I wherein R^{L} is optionally substituted

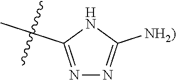

or a salt thereof.
In some embodiments, the two compounds are otherwise identical except that prior to performing the method, R^{L} is —COOH or a salt form or an activated form (e.g., anhydride) thereof while after performing the method in a produced compound R^{L} is optionally substituted

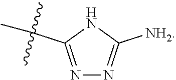 [SMILES: *c1nnc(N)[nH]1]

In some embodiments, R^{L} is

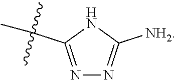 [SMILES: *c1nnc(N)[nH]1]

Various technologies are available to those skilled in the art and can be utilized in accordance with the present disclosure to perform a method. In some embodiments, an activated form of —COOH is an anhydride. In some embodiments, it is —C(O)OC(O)R′ wherein R′ is as described herein. In some embodiments, it is —C(O)OC(O)R wherein R is as described herein and is not —H. In some embodiments, R is C₁₋₆ aliphatic. In some embodiments, R is isopropyl. In some embodiments, a hydrazinecarboximidamide agent is optionally substituted NH₂C(NH)NHNH₂ or a salt thereof. In some embodiments, a hydrazinecarboximidamide agent is NH₂C(NH)NHNH₂ or a salt thereof. In some embodiments, a hydrazinecarboximidamide agent is hydrazinecarboximidamide bicarbonate. In some embodiments, a reaction is performed at a temperature above room temperature (e.g., at about 110° C.).

As appreciated by those skilled in the art, compounds comprising amino groups, e.g., in compounds of formula I wherein R^{L} is or comprises an amino group (e.g., wherein R^{L} is —NHR wherein R is C₁₋₆ aliphatic or —H, or is

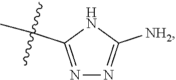 [SMILES: *c1nnc(N)[nH]1]

etc.) or salt forms thereof can be developed into many other compounds. For example, in some embodiments, an amino group may be converted into an amide group, a sulfonamide group, etc.

In some embodiments, the present disclosure provides a method comprising:
contacting a compound comprising an optionally substituted amino group and moiety A (e.g., a compound of formula I wherein R^{L} is or comprise —NHR′ or is

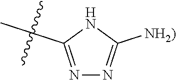

or a salt thereof with an acylation agent or sulfonylation agent; and
producing a compound comprising an amide or sulfonamide moiety and moiety A (e.g., a compound of formula I wherein R^{L} is —N(R′)C(O)) or a salt thereof.
   In some embodiments, the two compounds are otherwise identical except that after performing the method an optionally substituted amino group is replaced with an amide or sulfonamide group. In some embodiments, an amino group is —NHR wherein R is as described herein. In some embodiments, an amino group is —NH₂. In some embodiments, a compound is a compound of formula I or a salt thereof, wherein R^{L} is —NHR wherein R is as described herein. In some embodiments, a compound is a compound of formula I or a salt thereof, wherein R^{L} is —NH₂. In some embodiments, R^{L} is —N(R′)C(NR′)NHR′. In some embodiments, R^{L} is —NHC(NR′)NHR′. In some embodiments, R^{L} is —N(R′)C(NH)NHR′. In some embodiments, R^{L} is —NHC(NH)NHR′. In some embodiments, R^{L} is —NHC(NH)NH₂. In some embodiments, a compound is a compound of formula I or a salt thereof, wherein R^{L} is optionally substituted

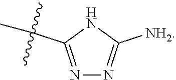 [SMILES: *c1nnc(N)[nH]1]

In some embodiments, a compound is a compound of formula I or a salt thereof, wherein R^{L} is

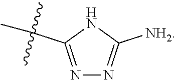 [SMILES: *c1nnc(N)[nH]1]

In some embodiments, an acylating agent is utilized and a compound comprising an amide moiety is formed. In some embodiments, an acylation agent has the structure of LG-C(O)R^{s} wherein LG is a leaving group (e.g., —Cl, —OH, etc.). In some embodiments, an acylating agent is RSC(O)OH or a salt or an activated form (e.g., anhydride, acyl halide, etc.). In some embodiments, R^{s} is R′ as described herein. In some embodiments, R′ is not —H. In some embodiments, in a produced compound R^{L} is —N(R′)C(O)R^{s} wherein R′ and R^{s} are independently as described herein. In some embodiments, in a produced compound R^{L} is —NHC(O)R^{s} wherein R^{s} is as described herein. In some embodiments, in a produced compound R^{L} is —NHC(O)R′ wherein R′ is as described herein. In some embodiments, in a produced compound R^{L} is

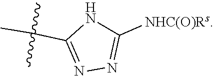

wherein R^{s} is as described herein. In some embodiments, in a produced compound R^{L} is

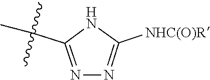

wherein R′ is as described herein. In some embodiments, a sulfonylation agent is utilized and a sulfonamide moiety is formed. In some embodiments, a sulfonylation agent is a compound having the structure of RsS(O)₂OH or a salt or activated form (e.g., sulfonyl chloride) thereof. In some embodiments, a sulfonylation agent is a sulfonyl chloride. In some embodiments, a sulfonylation is a compound having the structure of R^{s}S(O)₂Cl or a salt thereof. In some embodiments, R^{s} is R′ as described herein. In some embodiments, R′ is not —H. In some embodiments, in a produced compound R^{L} is —N(R′)S(O)₂R^{s} wherein R′ and R^{s} are independently as described herein. In some embodiments, in a produced compound R^{L} is —NHS(O)₂R^{s} wherein R^{s} is as described herein. In some embodiments, in a produced compound R^{L} is —NHS(O)₂R′ wherein R′ is as described herein. In some embodiments, in a produced compound R^{L} is

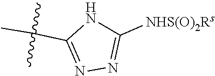

wherein R^{s} is as described herein. In some embodiments, in a produced compound R^{L} is

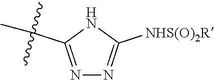

wherein R′ is as described herein. In some embodiments, in aproduced compound R^{L} is —N(R′)C(NR′)N(R′)S(O)₂R^{s}. In some embodiments, in a produced compound R^{L} is —NHC(NR′)N(R′)S(O)₂R^{s}. In some embodiments, in a produced compound R^{L} is —N(R′)C(NH)N(R′)S(O)₂R^{s}. In some embodiments, in a produced compound R^{L} is —NHC(NH)NHS(O)₂R^{s}. In some embodiments, in a produced compound R^{L} is —NHC(NH)NHS(O)₂R′. Various technologies are available to those skilled in the art and can be utilized in accordance with the present disclosure to perform a method. In some embodiments, a reaction is performed at a temperature below room temperature (e.g., at about 0° C.). In some embodiments, contacting is performed in the presence of a base, e.g., N(R)₃ (e.g., wherein each R is independently —H or optionally substituted C₁₋₆ alkyl such as Et₃N).

In some embodiments, the present disclosure provides a method comprising:
contacting a compound comprising an optionally substituted amino group and moiety A (e.g., a compound of formula I wherein R^{L} is or comprises —NHR′) or a salt thereof with an agent having the structure of N(R^{s})₂C(NR′)-LG or a salt thereof wherein LG is a leaving group and each of R′ and R^{s} is independently as described herein; and
producing a compound comprising —N(R′)C(NR′)N(R^{s})₂ and moiety A (e.g., a compound of formula I wherein R^{L} is —N(R′)C(NR′)N(R^{s})₂) or a salt thereof.
   In some embodiments, the two compounds are otherwise identical except that after performing the method —NHR′ is replaced with —N(R′)C(NR′)N(R^{s})₂. In some embodiments, R′ in —NHR′ is —H or optionally substituted C₁₋₆ aliphatic. In some embodiments, —NHR′ is —NH₂. In some embodiments, LG is an optionally substituted heteroaryl comprising at least one nitrogen bonded to N(R^{s})₂C(NR′)—. In some embodiments, LG is optionally substituted

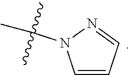 [SMILES: *n1cccn1]

In some embodiments, LG is optionally substituted

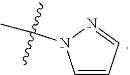 [SMILES: *n1cccn1]

In some embodiments, N(R^{s})₂C(NR′)— is N(R′)₂C(NR′)— wherein each R′ is independently as described herein. In some embodiments, it is N(R^{s})₂C(NH)— wherein each R^{s} is independently as described herein. In some embodiments, it is N(R′)₂C(NH)— wherein each R′ is independently as described herein. In some embodiments, it is NH₂C(NH)—. In some embodiments, —N(R′)C(NR′)N(R^{s})₂ is —NHC(NR′)N(R^{s})₂ wherein each of R′ and R^{s} is independently as described herein. In some embodiments, —C(NR′)N(R^{s})₂ is as described herein. Various technologies are available to those skilled in the art and can be utilized in accordance with the present disclosure to perform a method. In some embodiments, a reaction is performed at about room temperature. In some embodiments, contacting is performed in the presence of a base, e.g., N(R)₃ (e.g., wherein each R is independently —H or optionally substituted C₁₋₆ alkyl such as DIPEA).

In some embodiments, the present disclosure provides a method comprising:
contacting a compound comprising —CN and moiety A (e.g., a compound of formula I wherein R^{L} is —CN) or a salt thereof with an azide agent or a salt thereof; and
producing a compound comprising a tetrazolyl moiety and moiety A (e.g., a compound of formula I wherein R^{L} is optionally substituted

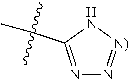

or a salt thereof.
In some embodiments, an azide agent is a compound of R^{s}—N₃ or a salt thereof. In some embodiments, an azide agent is TMSN₃. In some embodiments, in a produced compound R^{L} is

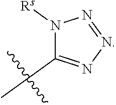

In some embodiments, in a produced compound R^{L} is

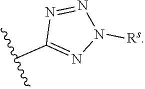

In some embodiments, R^{L} is optionally substituted

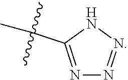 [SMILES: *c1nnn[nH]1]

In some embodiments, R^{L} is

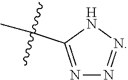 [SMILES: *c1nnn[nH]1]

Various technologies are available to those skilled in the art to perform a method in accordance with the present disclosure. For example, in some embodiments, contacting is performed in the presence of Bu₂SnO. In some embodiments, a reaction is performed in a suitable solvent. In some embodiments, a solvent is or comprises toluene. In some embodiments, a reaction is performed at a temperature higher that room temperature (e.g., at about 110° C., 120° C., etc.). In some embodiments, one or more groups may be protected, for example, in some embodiments, —OH may be protected as an ester (e.g., —OC(O)R′ such as AcO—). In some embodiments, after a reaction one or more protected groups, e.g., —OC(O)R′, are de-protected, e.g., to unprotected —OH.

In some embodiments, as appreciated by those skilled in the art, a —OH group such as R^{L} may be useful in various further reactions and/or transformations. For example, in some embodiments, the present disclosure provides a method comprising:
contacting a compound comprising —OH and moiety A (e.g., a compound of formula I wherein R^{L} is —OH) or a salt thereof to provide a —OC(O)— and moiety A (e.g., a compound of formula I wherein R^{L} comprises —OC(O)—, e.g., when R^{L} is —OC(O)N(R′)S(O)₂R^{s}) or a salt thereof.
   In some embodiments, the two compounds are otherwise identical except that prior to performing the method, R^{L} is —OH or a salt form thereof while after performing the method in a produced compound R^{L} is —OC(O)N(R′)S(O)₂R^{s} wherein R^{s} is as described herein. In some embodiments, R^{L} is —OC(O)NHS(O)₂R^{s}. In some embodiments, contacting is performed in the presence of an agent comprising —C(O)-LG, wherein LG is a leaving group as described herein. In some embodiments, LG is —OR wherein R is optionally substituted phenyl. In some embodiments, LG is —OPh. Various technologies are available to those skilled in the art and can be utilized in accordance with the present disclosure to perform a method. In some embodiments, contacting is conducted in the presence of an acylating agent and/or a base (e.g., N(R)₃ wherein each R is independently —H or optionally substituted C₁₋₆ aliphatic such as Et₃N). In some embodiments, an acylating agent is a compound has the structure of LG-C(O)R′ or a salt thereof, wherein LG is a leaving group and R′ is as described herein. In some embodiments, an acylating agent is a compound has the structure of LG-C(O)R^{L} or a salt thereof, wherein LG is a leaving group and R^{L} is as described herein. In some embodiments, an agent is a compound having the structure of LG-C(O)N(R′)S(O)₂R^{s} wherein each variable is independently as described herein. In some embodiments, LG is —Cl. In some embodiments, LG is —OR where R is as described herein and is not —H. In some embodiments, LG is —OR wherein R is optionally substituted phenyl. In some embodiments, LG is —OPh. In some embodiments, an agent is a compound having the structure of PhO—C(O)N(R′)S(O)₂R^{s} wherein each variable is independently as described herein. In some embodiments, a reaction is performed in a suitable solvent, e.g., a solvent which is or comprises THF. In some embodiments, a reaction is performed at a temperature about room temperature.

In some embodiments, the present disclosure provides technologies for preparing various useful agents, e.g., various sulfonamide agents. For example, in some embodiments, the present disclosure provides a method, comprising:
contacting a compound having the structure of HO—C(O)-L″-Cy-S(O)₂N(R′)₂ or a salt thereof with a reducing agent to provide a compound having the structure of HO—CH₂-L″-Cy-S(O)₂N(R′)₂ or a salt thereof, wherein each variable is independently as described herein.
   In some embodiments, L″ is optionally substituted —CH₂— and -Cy- is an optionally substituted aromatic ring. In some embodiments, L″ is —C(R′)₂— and -Cy- is an optionally substituted aromatic ring. In some embodiments, L″ is —C(R′)₂ wherein each R′ is independently as described herein and is not —H. In some embodiments, L″ s —C(R′)₂ wherein each R′ is independently optionally substituted C₁₋₆ aliphatic. In some embodiments, L″ is —C(CH₃)₂—. In some embodiments, -Cy- is optionally substituted phenylene. In some embodiments, -Cy- is optionally substituted 1, 4-phenylene. In some embodiments, -Cy- is 1, 4-phenylene. In some embodiments, each R′ of —S(O)₂N(R′)₂ is independently R as described herein. In some embodiments, —S(O)₂N(R′)₂ is —S(O)₂NH₂. In some embodiments, a compound reduced is

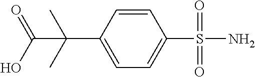 [SMILES: CC(C)(C(=O)O)c1ccc(S(N)(=O)=O)cc1]

or a salt thereof. In some embodiments, a product is

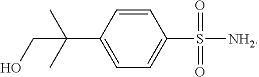

In some embodiments, a reducing agent is NaBH₄. In some embodiments, conducting is performed in the presence of a Lewis acid. In some embodiments, conducting is performed in the presence of BF₃. In some embodiments, conducting is performed in the presence of BF₃/Et₂O. In some embodiments, a reducing agent is NaBH₄ in the presence of BF₃/Et₂O. In some embodiments, contacting is performed in a suitable solvent, e.g., THF. In some embodiments, a reaction is performed at a temperature below room temperature (e.g., 0° C.). In some embodiments, a reaction is performed at about room temperature.

As appreciated by those skilled in the art, in chemical reactions various groups, e.g., hydroxyl, amino, carboxyl, etc. may be protected to avoid undesired reactions. Many technologies for protection/de-protection are available to those skilled in the art and may be utilized in accordance with the present disclosure. Certain such technologies are described herein including exemplified in the Examples.

Various chemical reactions are typically performed in a solvent. In some embodiments, a reaction is performed in a single solvent, e.g., DCM, THF, Et₂O, EtOH, toluene, etc. In some embodiments, a reaction is performed in a mixture of two or more solvents. In some embodiments, a solvent is polar. In some embodiments, a solvent is non-polar. In some embodiments, a solvent is protic. In some embodiments, a solvent is non-protic. In some embodiments, a solvent is polar but is not protic. Suitable solvents for various reactions are available to those skilled in the art and can be utilized in accordance with the present disclosure.

In some embodiments, a reaction is conducted under an inert atmosphere, e.g., N₂, Ar, etc. In some embodiments, a reaction is conducted with exposure to air. In some embodiments, a reaction is conducted under anhydrous conditions, e.g., with reagents, solvents, vessels, etc., properly dried. In some embodiments, a reaction is conducted in the presence of significant of water (e.g., about or more than about 0.1, 0.5, or 1 equivalent).

Reactions may be performed at various temperatures. In some embodiments, a reaction is performed at a temperature lower than about room temperature, e.g., about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about room temperature. In some embodiments, a reaction temperature is higher than room temperature. In some embodiments, a reaction temperature is about or at least about 35, 40, 50, 60, 70, 80, 90, 100, 100, 110, 120, 150° C., etc. In some embodiments, a reaction comprises refluxing in a solvent, e.g., in ether, toluene, etc. In some embodiments, temperature changes during a reaction process, e.g., increasing from a lower temperature to a higher temperature, decreasing from a higher temperature to a lower temperature, or both.

In some embodiments, the present disclosure provides compounds of high purity. In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 85%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.7%. In some embodiments, purity of a compound is or greater than about 99.9%.

In some embodiments, a product is selectively produced over another potential product. In some embodiments, a product is produced with chemoselectivity, stereoselectivity and/or regioselectivity. In some embodiments, a selectivity is presented as a ratio, e.g., of one product over another. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more.

Reactions may be performed for a variety of time lengths. In some embodiments, reactions complete instantly. In some embodiments, reaction times varies from minutes to hours to days, e.g., 5, 10, 15, 20, 30, 45 minutes, or 1, 1.5, 2, 3, 4, 5, 6, 7, 8, 9, 10, 12, 15, 18, 20 or 22 hours, or one or two days or longer. Those skilled in the art can use various technologies to determine when to terminate reactions, e.g., based on consumption of starting materials, products formation, by-products formation, etc. Characterization and Assessment

Those skilled in the art reading the present disclosure will appreciate that various technologies are available and may be utilized to assess provided technologies, e.g., compounds, compositions, methods, etc. Certain useful technologies are described in the Examples herein. In some embodiments, provided technologies are assessed in intro. In some embodiments, provided technologies are assessed in vivo. In some embodiments, provided technologies are assessed in animal models for conditions, disorders or diseases. In some embodiments, provided technologies are assessed in clinical trials involving human subjects. Certain useful technologies are described in, e.g., WO 2016073767, WO 2016086115, WO 2016086134, WO 2016086169, WO 2016086218, WO 2016130809, WO 2016161003, WO 2017147137, WO 2017147159, WO 2017147174, WO 2017189651, WO 2017189652, WO 2017189663, WO 2017201150, WO 2017201152, WO 2017201155, WO 2018067704, WO 2018081285, WO 2018102418, WO 2018152171, WO 2018187804, WO 2019118571, WO 2019160813, WO 2020231917, WO 2016/173524, Yu et al. eLife 2019; 8:e48431, etc. In some embodiments, technologies, e.g., for assessing activation of FXR, TGR5, and/or MRGPRX4 are described in Yu et al. eLife 2019; 8:e48431, etc. and are incorporated herein by reference. In some embodiments, a technology comprises utilization of cell lines. In some embodiments, a technology comprises administration of a compound to a subject, e.g., a human, an animal (e.g., mouse, monkey, etc.). In some embodiments, a technology comprises an animal model.

Among other things, the present disclosure provides technologies (e.g., compounds, compositions, methods, etc.) for selectively activate FXR and/or TGR5 over MRGPRX4. In some embodiments, selectivity is represented as ratios of EC50 values: EC50 for FXR and/or TGR5/EC50 MRGPRX4. In some embodiments, provided compounds provide higher selectivity than a reference compound. In some embodiments, a reference compound is obeticholic acid or a salt thereof. In some embodiments, a reference compound is cholic acid or a salt thereof. Those skilled in the art appreciate that to compare compound selectivities, compounds are generally assessed under the same or comparable conditions. In some embodiments, selectivity of a compound for FXR over MRGPRX4 is at least 2, 5, 10, 20, 50, 100, 200, 500, 1000 or more fold of a reference compound. In some embodiments, selectivity of a compound for TGR5 over MRGPRX4 is about or at least about 2, 5, 10, 20, 50, 100, 200, 500, 1000 or more fold of a reference compound. In some embodiments, selectivity of a compound for FXR and TGR5 over MRGPRX4 is independently about or at least about 2, 5, 10, 20, 50, 100, 200, 500, 1000 or more fold of a reference compound. In some embodiments, it is about or at least about 2 fold. In some embodiments, it is about or at least about 5 fold. In some embodiments, it is about or at least about 10 fold. In some embodiments, it is about or at least about 20 fold. In some embodiments, it is about or at least about 50 fold. In some embodiments, it is about or at least about 100 fold. In some embodiments, it is about or at least about 200 fold. In some embodiments, it is about or at least about 500 fold. In some embodiments, it is about or at least about 1000 fold. Additionally and alternatively, a compound provide comparable or higher activation level (e.g., as assessed using saturated/plateau levels in dosage curves) compared to a reference compound, e.g., obeticholic acid or a salt thereof, cholic acid or a salt thereof, etc. In some embodiments, a level is about or at least about 80% of a reference level. In some embodiments, it is about or at least about 85%. In some embodiments, it is about or at least about 90%. In some embodiments, it is about or at least about 100%. In some embodiments, it is about or at least about 1.1 fold. In some embodiments, it is about or at least about 1.2 fold. In some embodiments, it is about or at least about 1.5 fold. In some embodiments, it is about or at least about 2 fold. In some embodiments, it is about or at least about 2.5 fold. In some embodiments, it is about or at least about 3 fold. In some embodiments, it is about or at least about 4 fold. In some embodiments, it is about or at least about 5 fold.

Among other things, the present disclosure encompasses the recognition that side effects and/or adverse reactions associated with administration of bile acids and derivatives thereof may be associated with activation of MRGPRX4. In some embodiments, the present disclosure provides technologies for identifying compounds with lower side effects and/or adverse reactions.

In some embodiments, the present disclosure provides technologies for assessing compounds, e.g., those comprising moiety A, bile acids and derivatives and salts thereof, etc. In some embodiments, the present disclosure provides a method comprising
assessing activation of FXR and/or TGR5 by a compound and a reference compound;
assessing activation of MRGPRX4 by the compound and the reference compound;
wherein the compound provides higher selectivity for the activation of FXR and/or TGR5 over MRGPRX4.

In some embodiments, selectivity of a compound, as described herein, is about or at least about 2, 5, 10, 20, 50, 100, 200, 500, or 1000 fold or more compared to a reference compound. In some embodiments, activity of a compound for FXR and/or TGR5 is comparable or higher than a reference compound. In some embodiments, a compound comprises moiety A. In some embodiments, a compound is a bile acid, a bile acid derivative, or a salt thereof. In some embodiments, a reference compound is obeticholic acid or a salt thereof. In some embodiments, a reference compound is cholic acid or a salt thereof. In some embodiments, a compound is described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, WO 2016/173524, US20160145295A1, WO2017129125, Xian et al., ACS Med. Chem. Lett. 2017, 8, 1246-1251, et al., the compounds of each of which is incorporated herein by reference.

In some embodiments, a provided compound can provide sufficient levels of desired effects, e.g., activation of FXR and/or TGR5, without activating MRGPRX4.

In some embodiments, EC50 for FXR and/or TGR5 of a compound is about or no more than about 100, 50, 20, 10, 5, 4, 3, 2, 1, 0.5, 0.2, 0.1 or 0.05 uM. In some embodiments, it is about or no more than about 10 uM. In some embodiments, it is about or no more than about 5 uM. In some embodiments, it is about or no more than about 2 uM. In some embodiments, it is about or no more than about 1 uM. In some embodiments, it is about or no more than about 0.5 uM. In some embodiments, it is about or no more than about 0.2 uM. In some embodiments, it is about or no more than about 0.1 uM. In some embodiments, EC50 for MRGPRX4 of a compound is about or at least about 1, 2, 5, 10, 20, 50, 200, 500 or 1000 uM. In some embodiments, it is about or at least 1 uM. In some embodiments, it is about or at least 2 uM. In some embodiments, it is about or at least 5 uM. In some embodiments, it is about or at least 10 uM. In some embodiments, it is about or at least 20 uM. In some embodiments, it is about or at least 50 uM. In some embodiments, it is about or at least 100 uM. In some embodiments, under comparable conditions EC50 for FXR and/or TGR5 is about or no more than about 10 uM, and EF50 for MRGPRX4 is about or at least about 10 uM. In some embodiments, under comparable conditions EC50 for FXR and/or TGR5 is about or no more than about 10 uM, and EF50 for MRGPRX4 is about or at least about 50 uM. In some embodiments, under comparable conditions EC50 for FXR and/or TGR5 is about or no more than about 10 uM, and EF50 for MRGPRX4 is about or at least about 100 uM. In some embodiments, under comparable conditions EC50 for FXR and/or TGR5 is about or no more than about 5 uM, and EF50 for MRGPRX4 is about or at least about 5 uM. In some embodiments, under comparable conditions EC50 for FXR and/or TGR5 is about or no more than about 5 uM, and EF50 for MRGPRX4 is about or at least about 10 uM. In some embodiments, under comparable conditions EC50 for FXR and/or TGR5 is about or no more than about 5 uM, and EF50 for MRGPRX4 is about or at least about 20 uM. In some embodiments, under comparable conditions EC50 for FXR and/or TGR5 is about or no more than about 5 uM, and EF50 for MRGPRX4 is about or at least about 50 uM. In some embodiments, under comparable conditions EC50 for FXR and/or TGR5 is about or no more than about 5 uM, and EF50 for MRGPRX4 is about or at least about 100 uM. In some embodiments, under comparable conditions EC50 for FXR and/or TGR5 is about or no more than about 1 uM, and EF50 for MRGPRX4 is about or at least about 1 uM. In some embodiments, under comparable conditions EC50 for FXR and/or TGR5 is about or no more than about 1 uM, and EF50 for MRGPRX4 is about or at least about 10 uM. In some embodiments, under comparable conditions EC50 for FXR and/or TGR5 is about or no more than about 1 uM, and EF50 for MRGPRX4 is about or at least about 20 uM. In some embodiments, under comparable conditions EC50 for FXR and/or TGR5 is about or no more than about 1 uM, and EF50 for MRGPRX4 is about or at least about 50 uM. In some embodiments, under comparable conditions EC50 for FXR and/or TGR5 is about or no more than about 1 uM, and EF50 for MRGPRX4 is about or at least about 100 uM.

In some embodiments, EC50 of a provided compound for MRGPRX4 is about or at least about 1, 2, 3, 4, 5, 6, 7, 8, 9, 10, 11, 12, 13, 14, 15, 16, 17, 18, 19, 20, 30, 40, 50, 60, 70, 80, 90, 100, 200, 500 or 1000 fold of that of a reference compound. In some embodiments, it is about or at least about 2 fold. In some embodiments, it is about or at least about 5 fold. In some embodiments, it is about or at least about 10 fold. In some embodiments, it is about or at least about 20 fold. In some embodiments, it is about or at least about 50 fold. In some embodiments, it is about or at least about 100 fold.

In some embodiments, a compound activates FXR at a level that is about or at least about 10%, 20%, 25%, 30%, 40%, 50%, 60%, 70%, 75%, 80%, 90%, 100%, 125%, 150%, 200%, 250%, 300%, 350%, 400%, 450%, or 500% of that of a reference compound at a concentration. In some embodiments, it is about or at least about 50%. In some embodiments, it is about or at least about 100%. In some embodiments, it is about or at least about 150%. In some embodiments, it is about or at least about 200%. In some embodiments, it is about or at least about 250%. In some embodiments, it is about or at least about 300%. In some embodiments, it is about or at least about 350%. In some embodiments, it is about or at least about 400%. In some embodiments, a concentration is about or at least about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM. In some embodiments, a concentration is about or at least about 100 uM. In some embodiments, a concentration is about or at least about 80 uM. In some embodiments, a concentration is about or at least about 50 uM. In some embodiments, a concentration is about or at least about 20 uM. In some embodiments, a concentration is about or at least about 10 uM. In some embodiments, a concentration is about or at least about 5 uM. In some embodiments, a concentration is about or at least about 2 uM. In some embodiments, a concentration is about or at least about 1 uM. In some embodiments, a concentration is about or at least about 0.5 uM. In some embodiments, a concentration is about or at least about 0.2 uM. In some embodiments, a concentration is about or at least about 0.1 uM. In some embodiments, a concentration is about or at least about 0.05 uM. In some embodiments, a concentration is about or at least about 0.02 uM. In some embodiments, a concentration is about or at least about 0.01 uM. In some embodiments, a concentration is about or at least about a concentration described in an Example. In some embodiments, a concentration is about or at least about EC10, EC20, EC30, EC40, EC50, EC60, EC70, EC80, EC90, or EC100 of a provided compound for FXR activation. In some embodiments, it is about or at least about EC50 of a provided compound for FXR activation. In some embodiments, a concentration is about or at least about EC10, EC20, EC30, EC40, EC50, EC60, EC70, EC80, EC90, or EC100 of a reference compound for FXR activation. In some embodiments, it is about or at least about EC50 of a reference compound for FXR activation. In some embodiments, a concentration is an effective concentration of a desired biological effect or application. In some embodiments, a concentration is effective for preventing or treating a condition, disorder or disease as described herein. In some embodiments, a concentration is provided by administering an effective amount of a compound for preventing or treating a condition, disorder or disease as described herein.

In some embodiments, a compound activates TGR5 at a level that is about or at least about 10%, 20%, 25%, 30%, 40%, 50%, 60%, 70%, 75%, 80%, 90%, 100%, 125%, 150%, 200%, 250%, 300%, 350%, 400%, 450%, or 500% of that of a reference compound at a concentration. In some embodiments, it is about or at least about 50%. In some embodiments, it is about or at least about 100%. In some embodiments, it is about or at least about 150%. In some embodiments, it is about or at least about 200%. In some embodiments, it is about or at least about 250%. In some embodiments, it is about or at least about 300%. In some embodiments, it is about or at least about 350%. In some embodiments, it is about or at least about 400%. In some embodiments, a concentration is about or at least about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM. In some embodiments, a concentration is about or at least about 100 uM. In some embodiments, a concentration is about or at least about 80 uM. In some embodiments, a concentration is about or at least about 50 uM. In some embodiments, a concentration is about or at least about 20 uM. In some embodiments, a concentration is about or at least about 10 uM. In some embodiments, a concentration is about or at least about 5 uM. In some embodiments, a concentration is about or at least about 2 uM. In some embodiments, a concentration is about or at least about 1 uM. In some embodiments, a concentration is about or at least about 0.5 uM. In some embodiments, a concentration is about or at least about 0.2 uM. In some embodiments, a concentration is about or at least about 0.1 uM. In some embodiments, a concentration is about or at least about 0.05 uM. In some embodiments, a concentration is about or at least about 0.02 uM. In some embodiments, a concentration is about or at least about 0.01 uM. In some embodiments, a concentration is about or at least about a concentration described in an Example. In some embodiments, a concentration is about or at least about EC10, EC20, EC30, EC40, EC50, EC60, EC70, EC80, EC90, or EC100 of a provided compound for TGR5 activation. In some embodiments, it is about or at least about EC50 of a provided compound for TGR5 activation. In some embodiments, a concentration is about or at least about EC10, EC20, EC30, EC40, EC50, EC60, EC70, EC80, EC90, or EC100 of a reference compound for TGR5 activation. In some embodiments, it is about or at least about EC50 of a reference compound for TGR5 activation. In some embodiments, it is about or at least about EC50 of a reference compound for FXR activation. In some embodiments, a concentration is an effective concentration of a desired biological effect or application. In some embodiments, a concentration is effective for preventing or treating a condition, disorder or disease as described herein. In some embodiments, a concentration is provided by administering an effective amount of a compound for preventing or treating a condition, disorder or disease as described herein.

In some embodiments, MRGPRX4 activation by a provided compound is about or no more than about 1%, 2%, 5%, 10%, 15%, 20%, 25%, 30%, 40% or 50% of that of a reference compound at a concentration. In some embodiments, it is about or no more than about 50%. In some embodiments, it is about or no more than about 25%. In some embodiments, it is about or no more than about 20%. In some embodiments, it is about or no more than about 10%. In some embodiments, it is about or no more than about 5%. In some embodiments, it is about or no more than about 2%. In some embodiments, it is about or no more than about 1%. In some embodiments, no MRGPRX4 activation by a provided compound is observed at a concentration which MRGPRX4 activation by a reference compound is observed. In some embodiments, a concentration is about or at least about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM. In some embodiments, a concentration is about or at least about 100 uM. In some embodiments, a concentration is about or at least about 80 uM. In some embodiments, a concentration is about or at least about 50 uM. In some embodiments, a concentration is about or at least about 20 uM. In some embodiments, a concentration is about or at least about 10 uM. In some embodiments, a concentration is about or at least about 5 uM. In some embodiments, a concentration is about or at least about 2 uM. In some embodiments, a concentration is about or at least about 1 uM. In some embodiments, a concentration is about or at least about 0.5 uM. In some embodiments, a concentration is about or at least about 0.2 uM. In some embodiments, a concentration is about or at least about 0.1 uM. In some embodiments, a concentration is about or at least about 0.05 uM. In some embodiments, a concentration is about or at least about 0.02 uM. In some embodiments, a concentration is about or at least about 0.01 uM. In some embodiments, a concentration is about or at least about a concentration described in an Example. In some embodiments, a concentration is about or at least about EC10, EC20, EC30, EC40, EC50, EC60, EC70, EC80, EC90, or EC100 of a reference compound for MRGPRX4 activation. In some embodiments, it is about or at least about EC10 of a reference compound for MRGPRX4 activation. In some embodiments, it is about or at least about EC20 of a reference compound for MRGPRX4 activation. In some embodiments, it is about or at least about EC30 of a reference compound for MRGPRX4 activation. In some embodiments, it is about or at least about EC40 of a reference compound for MRGPRX4 activation. In some embodiments, it is about or at least about EC50 of a reference compound for MRGPRX4 activation. In some embodiments, it is about or at least about EC60 of a reference compound for MRGPRX4 activation. In some embodiments, it is about or at least about EC70 of a reference compound for MRGPRX4 activation. In some embodiments, it is about or at least about EC80 of a reference compound for MRGPRX4 activation. In some embodiments, it is about or at least about EC90 of a reference compound for MRGPRX4 activation. In some embodiments, it is about or at least about EC100 of a reference compound for MRGPRX4 activation. In some embodiments, MRGPRX4 activation by a provided compound is observed. In some embodiments, a concentration is about or at least about EC10, EC20, EC30, EC40, EC50, EC60, EC70, EC80, EC90, or EC100 of a compound for MRGPRX4 activation. In some embodiments, it is about or at least about EC10 of a compound for MRGPRX4 activation. In some embodiments, it is about or at least about EC20 of a compound for MRGPRX4 activation. In some embodiments, it is about or at least about EC30 of a compound for MRGPRX4 activation. In some embodiments, it is about or at least about EC40 of a compound for MRGPRX4 activation. In some embodiments, it is about or at least about EC50 of a compound for MRGPRX4 activation. In some embodiments, it is about or at least about EC60 of a compound for MRGPRX4 activation. In some embodiments, it is about or at least about EC70 of a compound for MRGPRX4 activation. In some embodiments, it is about or at least about EC80 of a compound for MRGPRX4 activation. In some embodiments, it is about or at least about EC90 of a compound for MRGPRX4 activation. In some embodiments, it is about or at least about EC100 of a compound for MRGPRX4 activation. In some embodiments, a concentration is about or at least about 1, 2, 3, 4, 5, 6, 7, 8, 9, 10, 11, 12, 13, 14, 15, 16, 17, 18, 19, 20, 30, 40, 50, 60, 70, 80, 90, or 100 fold of the EC50 for FXR or TGR5 by the compound. In some embodiments, it is for FXR. In some embodiments, it is for TGR5. In some embodiments, it is at least 2 fold. In some embodiments, it is at least 5 fold. In some embodiments, it is at least 10 fold. In some embodiments, it is at least 20 fold. In some embodiments, it is at least 50 fold. In some embodiments, it is at least 100 fold. In some embodiments, it is at least 200 fold. In some embodiments, it is at least 500 fold. In some embodiments, it is at least 1000 fold.

Various reference compounds are described herein. For example, in some embodiments, a reference compound is a natural bile acid or a salt thereof. In some embodiments, a reference compound is DCA or a salt thereof. In some embodiments, a reference compound is OCA or a salt thereof. In some embodiments, a reference compound is CDCA or a salt thereof. In some embodiments, a reference compound is cholic acid or a salt thereof. In some embodiments, a reference compound has a 3-OH group as in a natural bile acid while a provided compound does not have a 3′-OH group. In some embodiments, none of R¹ and R^{1a} in the compound is —OH, and a reference compound is an otherwise identical compound except that one of R¹ and R^{1a} in the reference compound is —OH. In some embodiments, as described herein, in a provided compound both R¹ and R^{1a} is —H; in some embodiments, one of them is —H and the other is —F; in some embodiments, both are —F.

### Biological Applications

Among other things, provided technologies are useful for modulating various biological activities, e.g., those associated with bile acids and/or analogs and/or derivatives thereof. For example, in some embodiments, provided compounds are useful as FXR agonists. In some embodiments, provided compounds are useful as TGR5 agonists. Surprisingly, the present disclosure demonstrates that various provided compounds can serve as FXR and/or TGR5 agonists with reduced off-target binding, side effects, adverse reactions, etc., including those associated with binding to MRGPRX4. In some embodiments, provided compounds can provide improved EC50 and/or efficacy relative to a reference compound. In some embodiments, a reference compound is a bile acid. In some embodiments, a reference compound is CDCA. In some embodiments, a reference compound is OCA. In some embodiments, a reference compound is DCA. In some embodiments, a reference compound is a reported bile acid analog or derivative, e.g., a FXR agonist described in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, etc.

In some embodiments, EC50, e.g., as measured according to procedures described in the Examples herein, of a provided compound for MRGPRX4 is at least about 50, 60, 70, 80, 90, or 100 μM. In some embodiments, EC50, e.g., as measured according to procedures described in the Examples herein, of a provided compound for FXR is no more than about 1, 2, 3, 4, 5, 6, 7, 8, 9, or 10 μM. In some embodiments, EC50 for FXR is no more than 10 μM. In some embodiments, EC50 for FXR is no more than 1 μM. In some embodiments, EC50, e.g., as measured according to procedures described in the Examples herein, of a provided compound for FXR is no more than about 0.01, 0.02, 0.05, 0.1, 0.2, 0.5, 1, 2, 3, 4, 5, 6, 7, 8, 9, or 10 μM. In some embodiments, EC50 for TGR5 is no more than 0.02 μM. In some embodiments, EC50 for TGR5 is no more than 0.05 μM. In some embodiments, EC50 for TGR5 is no more than 0.1 μM. In some embodiments, EC50 for TGR5 is no more than 0.2 μM. In some embodiments, EC50 for TGR5 is no more than 0.5 μM. In some embodiments, EC50 for TGR5 is no more than 1 μM. In some embodiments, EC50 for TGR5 is no more than 2 μM. In some embodiments, EC50 for TGR5 is no more than 5 μM. In some embodiments, EC50 for TGR5 is no more than 10 μM.

### Farnesoid X Receptor (FXR)

It has been reported that Farnesoid X Receptor (FXR) is an orphan nuclear receptor initially identified from a mat liver cDNA library (BM. Forman, et al, Cell, 1995, 81 (5, 687-693) that is most closely related to an insect ecdysone receptor. FXR is reportedly a member of a nuclear receptor family of ligand activated transcription factors that includes receptors for, e.g., steroid, retinoid, and thyroid hormones (DJ. Mangelsdorf, et al, Cell, 1995, 83(6), 841-850). Reported relevant physiological ligands of FXR include bile acids (D Parks et al, Science, 1999, 284(5418), 1362-1365). One of the most potent ones is reported to be chenodeoxycholic acid (CDCA), which has been reported to regulate expression of several genes that participate in bile acid homeostasis. Famesol and derivatives, together called farnesoids, are originally described to activate a rat orthologue at high concentrations but they are not reported to similarly activate corresponding human or mouse receptors. FXR has been reported to express in various cells, tissues and organs, e.g., liver and throughout entire gastrointestinal tract, including esophagus, stomach, duodenum, small intestine, colon, ovary, adrenal gland and kidney. In addition to controlling gene expression, FXR has also been suggested to be involved in paracrine and endocrine signaling by up regulating expression of, e.g., cytokine Fibroblast Growth Factor (J. Holt et al., Genes Dev, 2003, 17(13), 1581-1591; T. Inagaki et al, Cell Metab, 2005, 2(4), 21225).

Various FXR modulators, including FXR agonists, have been reported, including those described in WO 2016073767, WO 2016086115, WO 2016086134, WO 2016086169, WO 2016086218, WO 2016130809, WO 2016161003, WO 2017147137, WO 2017147159, WO 2017147174, WO 2017189651, WO 2017189652, WO 2017189663, WO 2017201150, WO 2017201152, WO 2017201155, WO 2018067704, WO 2018081285, WO 2018102418, WO 2018152171, WO 2018187804, WO 2019118571, WO 2019160813, WO 2020231917, WO 2016/173524 and Yu et al. eLife 2019; 8:e48431. Many FXR modulators, e.g., those reported in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804, WO 2016/173524 and Yu et al. eLife 2019; 8:e48431 comprise 3-OH attached to moiety A.

Among other things, the present disclosure encompasses the recognition that such 3-OH attached to moiety A is associated to various off-target effects (e.g., activation of MRGPRX4), side effects, and/or adverse reactions. In some embodiments, the present disclosure provides technologies for reducing such undesired effects by removal of such 3-OH. In some embodiments, such 3-OH is replaced with R¹ or R^{a} as described herein, wherein R¹ or R^{1a} is not —OH. In some embodiments, R¹ or R^{1a} is —H. In some embodiments, R¹ or R^{1a} is halogen, e.g., —F. In some embodiments, provided compounds, e.g., compounds of formula I or salts thereof wherein R¹ and R^{1a} are not —OH has improved therapeutic index and/or safety window compared to a reference compound which comprises 3-OH attached to moiety A. In some embodiments, a reference compound is a bile acid. In some embodiments, a reference compound is a 3-OH bile acid compound. In some embodiments, a reference compound is cholic acid. In some embodiments, a reference compound is obeticholic acid. In some embodiments, a reference compound is an otherwise identical compound but possesses 3-OH attached to moiety A.

In some embodiments, the present disclosure provides technologies for modulating FXR functions. In some embodiments, the present disclosure provides technologies for activating FXR. In some embodiments, the present disclosure provides methods for modulating a FXR function comprising contacting a compound, e.g., a compound of formula I or a salt thereof, with FXR. In some embodiments, the present disclosure provides methods for modulating a FXR function comprising administering or delivering a compound, e.g., a compound of formula I or a salt thereof, to a system (e.g., a cell, tissue, organ, organism, subject, etc.) comprising or expressing FXR. In some embodiments, a system is or comprises a population of cells within a tissue, organ, organism, or subject. In some embodiments, a system comprises or expresses TGR5. In some embodiments, a system comprises or expresses MRGPRX4. In some embodiments, a system does not comprise or express MRGPRX4. In some embodiments, provided technologies provided higher selectivity for FXR and/or TGR5 over MRGPRX4 (e.g., as demonstrated by lower EC50 for FXR and/or TGR5 compared to MRGPRX4). In some embodiments, provided technologies provided higher selectivity for FXR and/or TGR5 over MRGPRX4 (e.g., as demonstrated by higher ratios of EC50 for FXR and/or TGR5 over EC50 for MRGPRX4) compared to a reference compound (e.g., a bile acid, cholic acid, obeticholic acid, etc.). In some embodiments, the present disclosure provides technologies for preventing or treating a condition, disorder or disease associated with FXR, comprising administering or delivering to a subject susceptible thereto or suffering therefrom an effective amount of a compound of present disclosure, e.g., a compound of formula I or a salt thereof.

### TGR5

TGR5 receptor is reported to be a G-protein coupled receptor that has been identified as a cell-surface receptor that is responsive to bile acids (BAs). The primary structure of TGR5 and its responsiveness to bile acids has been reported to be highly conserved in TGR5 among human, bovine, rabbit, rat, and mouse. (Kawamata et al, J. Bio. Chem., 2003, 278, 9435). TGR5 has been reported to be identical to hGPCR19 reported by Takeda et al, FEBS Lett 2002, 520, 97-101.

Various FXR modulators, including FXR agonists, have been reported and be utilized as TGR5 agonist, for example, those described in WO 2016073767, WO 2016086115, WO 2016086134, WO 2016086169, WO 2016086218, WO 2016130809, WO 2016161003, WO 2017147137, WO 2017147159, WO 2017147174, WO 2017189651, WO 2017189652, WO 2017189663, WO 2017201150, WO 2017201152, WO 2017201155, WO 2018067704, WO 2018081285, WO 2018102418, WO 2018152171, WO 2018187804, WO 2019118571, WO 2019160813, WO 2020231917, WO 2016/173524 and Yu et al. eLife 2019; 8:e48431. Many such compounds, e.g., those reported in WO 2016073767, WO 2016086115, WO 2016086134, WO 2016086169, WO 2016086218, WO 2016130809, WO 2016161003, WO 2017147137, WO 2017147159, WO 2017147174, WO 2018102418, WO 2018152171, WO 2018187804 and Yu et al. eLife 2019; 8:e48431 comprise 3-OH attached to moiety A.

In some embodiments, the present disclosure provides technologies for modulating TGR5 functions. In some embodiments, the present disclosure provides technologies for activating TGR5. In some embodiments, the present disclosure provides methods for modulating a TGR5 function comprising contacting a compound, e.g., a compound of formula I or a salt thereof, with TGR5. In some embodiments, the present disclosure provides methods for modulating a TGR5 function comprising administering or delivering a compound, e.g., a compound of formula I or a salt thereof, to a system (e.g., a cell, tissue, organ, organism, subject, etc.) comprising or expressing TGR5. In some embodiments, a system is or comprises a population of cells within a tissue, organ, organism, or subject. In some embodiments, a system comprises or expresses FXR. In some embodiments, a system comprises or expresses MRGPRX4. In some embodiments, a system does not comprise or express MRGPRX4. In some embodiments, provided technologies provided higher selectivity for FXR and/or TGR5 over MRGPRX4 (e.g., as demonstrated by lower EC50 for FXR and/or TGR5 compared to MRGPRX4). In some embodiments, provided technologies provided higher selectivity for FXR and/or TGR5 over MRGPRX4 (e.g., as demonstrated by higher ratios of EC50 for FXR and/or TGR5 over EC50 for MRGPRX4) compared to a reference compound (e.g., a bile acid, cholic acid, obeticholic acid, etc.). In some embodiments, the present disclosure provides technologies for preventing or treating a condition, disorder or disease associated with TGR5, comprising administering or delivering to a subject susceptible thereto or suffering therefrom an effective amount of a compound of present disclosure, e.g., a compound of formula I or a salt thereof.

### MRGPRX4

MRGPRX4 is reported to be a bile acid receptor that may underlie cholestatic itch in human. See, e.g., Yu et al. eLife 2019; 8:e48431. Among other things, the present disclosure encompasses the recognition and side effects of various compounds comprising 3-OH attached to moiety A, e.g., various 3-OH bile acid compounds including those of clinical relevance such as cholic acid and obeticholic acid, including adverse reactions such as severe pruritus, can be reduced by removal of such 3-OH. In some embodiments, such 3-OH is replaced with R¹ or R^{1a} as described herein, wherein R¹ or R^{1a} is not —OH. In some embodiments, R¹ or R^{1a} is —H. In some embodiments, R¹ or R^{1a} is halogen, e.g., —F. In some embodiments, provided compounds, e.g., compounds of formula I or salts thereof wherein R¹ and R^{1a} are not —OH has higher selectivity for activation of FXR and/or TGR5 over MRGPRX4 compared to a reference compound. In some embodiments, provided compounds, e.g., compounds of formula I or salts thereof wherein R¹ and R^{1a} are not —OH has improved therapeutic index and/or safety window compared to a reference compound which comprises 3-OH attached to moiety A (e.g., with respect to one or more side effects/adverse reactions, e.g., pruritus). In some embodiments, a reference compound is a bile acid. In some embodiments, a reference compound is a 3-OH bile acid compound. In some embodiments, a reference compound is cholic acid. In some embodiments, a reference compound is obeticholic acid. In some embodiments, a reference compound is an otherwise identical compound but possesses 3-OH attached to moiety A.

In some embodiments, the present disclosure provides methods for preventing or treating various conditions, disorders or diseases wherein subjects susceptible thereto or suffering therefrom can benefit from increased levels of FXR and/or TGR5 activity. In some embodiments, the present disclosure provides methods for preventing or treating various conditions, disorders or diseases wherein subjects susceptible thereto or suffering therefrom can benefit from increased levels of FXR activity. In some embodiments, the present disclosure provides methods for preventing various conditions, disorders or diseases wherein subjects susceptible thereto can benefit from increased levels of FXR activity. In some embodiments, the present disclosure provides methods for treating various conditions, disorders or diseases wherein subjects suffering therefrom can benefit from increased levels of FXR activity. In some embodiments, the present disclosure provides methods for preventing or treating various conditions, disorders or diseases wherein subjects susceptible thereto or suffering therefrom can benefit from increased levels of TGR5 activity. In some embodiments, the present disclosure provides methods for preventing various conditions, disorders or diseases wherein subjects susceptible thereto can benefit from increased levels of TGR5 activity. In some embodiments, the present disclosure provides methods for treating various conditions, disorders or diseases wherein subjects suffering therefrom can benefit from increased levels of TGR5 activity.

In some embodiments, the present disclosure provides a method for preventing a condition, disorder or disease, comprising administering or delivering to a subject susceptible thereto an effective amount of a compound of the present disclosure or a pharmaceutically acceptable salt thereof. In some embodiments, the present disclosure provides a method for treating a condition, disorder or disease, comprising administering or delivering to a subject suffering therefrom an effective amount of a compound of the present disclosure or a pharmaceutically acceptable salt thereof. In some embodiments, onset of a condition, disorder or disease is delayed or prevented. In some embodiments, severity of a condition, disorder or disease is reduced. In some embodiments, a symptom of a condition, disorder or disease is prevented, removed or ameliorated. In some embodiments, a biomarker, score, etc. is improved.

In some embodiments, the present disclosure provides a method for preventing a condition, disorder or disease, comprising:
obtaining information that a compound selectively activate FXR and/or TGR5 over MRGPRX4 compared to a reference compound; and
administering or delivering to, or providing instructions to administer or deliver to, a subject susceptible thereto an effective amount of the compound.

In some embodiments, the present disclosure provides a method for treating a condition, disorder or disease, comprising:
obtaining information that a compound selectively activate FXR and/or TGR5 over MRGPRX4 compared to a reference compound; and
administering or delivering to, or providing instructions to administer or deliver to, a subject suffering therefrom an effective amount of the compound.

In some embodiments, obtaining is through education, certification, qualification, research, literature, lectures, advertisement, marketing materials, prescription information, etc. In some embodiments, a reference compound is a compound comprising 3-OH and moiety A. In some embodiments, a reference compound is obeticholic acid or a salt thereof. In some embodiments, a reference compound is cholic acid or a salt thereof. In some embodiments, an administered or delivered compound is a FXR agonist. In some embodiments, it is a TGR5 agonist. In some embodiments, it can provide clinical benefits without significant activation of MRGPRX4. In some embodiments, it can provide clinical benefits without significant side effects and/or adverse reactions associated with MRGPRX4 activation. In some embodiments, it can provide clinical benefits with reduced levels of side effects and/or adverse reactions associated with MRGPRX4 activation compared to a reference compound. In some embodiments, it is a bile acid or derivative thereof. In some embodiments, it is a compound provided herein (e.g., a compound or formula I or a salt thereof).

Those skilled in the art appreciate that provided technologies are useful for preventing or treating various conditions, disorders or diseases. In some embodiments, a condition, disorder or disease is nonalcoholic steatohepatitis (NASH). In some embodiments, a condition, disorder or disease is bile acid synthesis condition, disorder or disease. In some embodiments, a bile acid synthesis condition, disorder or disease is due to single enzyme defects (SEDs). In some embodiments, a compound may be utilized as an adjunctive treatment of a peroxisomal condition, disorder or disease, e.g., a Zellweger spectrum disorder. In some embodiments, a patient of a peroxisomal condition, disorder or disease, e.g., a Zellweger spectrum disorder, exhibit manifestations of a liver condition, disorder or disease, steatorrhea or complications from decreased fat-soluble vitamin absorption.

In some embodiments, a condition, disorder or disease is cardiovascular disease, atherosclerosis, arteriosclerosis, hypercholesteremia, hyperlipidemia, chronic liver disease, gastrointestinal disease, renal disease, metabolic disease, cancer (i.e., colorectal cancer), or neurological indications such as stroke. In certain embodiments, a chronic liver disease is primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency. In certain embodiments, a gastrointestinal disease is inflammatory bowel disease (IBD) (including Crohn's disease and ulcerative colitis), irritable bowel syndrome (IBS), bacterial overgrowth, malabsorption, post-radiation colitis, or microscopic colitis. In certain embodiments, the renal disease is diabetic nephropathy, focal segmental glomerulosclerosis (FSGS), hypertensive nephrosclerosis, chronic glomerulonephritis, chronic transplant glomerulopathy, chronic interstitial nephritis, or poly cystic kidney disease. In certain embodiments, a cardiovascular disease is atherosclerosis, arteriosclerosis, dyslipidemia, hypercholesterolemia, or hypertriglyceridemia. In certain embodiments, a metabolic disease is insulin resistance, Type I and Type II diabetes, or obesity. In some embodiments, a condition, disorder or disease is an inflammatory condition, disorder or disease, e.g., allergy, osteoarthritis, appendicitis, bronchial asthma, pancreatitis, allergic rash, psoriasis, etc. In some embodiments, a condition, disorder or disease is an autoimmune condition, disorder or disease. In some embodiments, a condition, disorder or disease is rheumatoid arthritis, multiple sclerosis, and type I diabetes. In some embodiments, a condition, disorder or disease is a gastrointestinal disease, e.g., inflammatory bowel disease (Crohn's disease, ulcerative colitis), short bowel syndrome (post-radiation colitis), microscopic colitis, irritable bowel syndrome (malabsorption), and bacterial overgrowth. In some embodiments, a condition, disorder or disease is cancer. In some embodiments, a cancer is colorectal cancer, liver cancer, hepatocellular carcinoma, cholangio carcinoma, renal cancer, gastric cancer, pancreatic cancer, prostate cancer, or insulanoma. In some embodiments, a condition, disorder or disease is FXR-mediated. In some embodiments, a condition, disorder or disease is TGR5-mediated. In some embodiments, subjects susceptible thereto and/or suffering therefrom benefit from increased levels of FXR and/or TGR5 activity. In some embodiments, a condition, disorder or disease is a neurodegenerative condition, disorder or disease. In some embodiments, a condition, disorder or disease is amyotrophic lateral sclerosis (ALS).

With improved properties, activities, selectivities and/or reduced off-target binding, side effects, adverse reactions, etc., provided technologies can provided improved dosing regimen (e.g., reduced single dose (e.g., in view of improved potency), increased single dose (e.g., in view of reduced toxicity), reduced total dose (e.g., in view of improved potency), increased total dose (e.g., in view of reduced toxicity), extended dosing intervals (e.g., in view of improved potency), reduced dosing intervals (e.g., in view of reduced toxicity), fewer doses (e.g., in view of improved potency), more doses (e.g., in view of reduced toxicity), shorter treatment period (e.g., in view of improved potency), extended treatment period (e.g., in view of reduced toxicity) and/or improved outcomes compared to reference technologies utilizing reference compounds.

In some embodiments, a provided compound is utilized in combination with an additional agent. In some embodiments, an additional agent is a therapeutic agent. In some embodiments, a provided compound is utilized in combination with two or more therapeutic agents. For example, in some embodiments, an additional agent is, comprises or delivers phenylbutyric acid or a salt thereof. In some embodiments, an additional agent is a phenylbutyrate pharmaceutically acceptable salt. In some embodiments, an additional agent is sodium phenylbutyrate. In some embodiments, a compound is 3-deoxy ursodoxicoltaurine (R¹ and R^{1a} are —H, otherwise identical with ursodoxicoltaurine (in which one of R¹ and R^{1a} is —H and the other is —OH).

Additionally or alternatively, in some embodiments, a compound is utilized in combination with a G-protein coupled receptor (GPCR) inhibitor. Certain relevant GPCRs, e.g., those related to pain and/or itch, and inhibitors thereof, are described in US 20210356455, the GPCRs, GPCR inhibitors and methods for identifying, characterizing, assessing, preparing and/or using GPCR inhibitors are incorporated herein by reference.

Additionally or alternatively, in some embodiments, a compound is utilized in combination with a MRGPRX inhibitor. In some embodiments, a compound is utilized in combination with a MRGPRX1 inhibitor. In some embodiments, a MRGPRX1 inhibitor can further reduce severity of or prevent a condition, disorder or disease, e.g., itch. Various MRGPRX1 inhibitors, e.g., those described in WO 2022/119823, the inhibitors and methods for identifying, characterizing, assessing, preparing and/or using the inhibitors are incorporated herein by reference. In some embodiments, a compound is utilized in combination with a MRGPRX2 inhibitor. In some embodiments, a MRGPRX2 inhibitor can further reduce severity of or prevent a condition, disorder or disease, e.g., itch. Various MRGPRX2 inhibitors, e.g., those described in WO 2022/111473, WO 2022/140520, WO 2022/125636, WO 2016/019246, US 20220340559, US 20220340530, WO 2022/087083, US 20200370051, WO 2021/092262, WO 2021/092264, US 20170204419, US 20220177434, WO 2021/092240, and WO 2022/067094, the inhibitors and methods for identifying, characterizing, assessing, preparing and/or using the inhibitors of each of which are independently incorporated herein by reference. In some embodiments, a compound is utilized in combination with a MRGPRX3 inhibitor. In some embodiments, a MRGPRX3 inhibitor can further reduce seventy of or prevent a condition, disorder or disease, e.g., itch. Various MRGPRX3 inhibitors, e.g., those described in WO 2022/079245, and WO 2018/232316, the inhibitors and methods for identifying, characterizing, assessing, preparing and/or using the inhibitors of each of which are independently incorporated herein by reference. In some embodiments, a compound is utilized in combination with a MRGPRX4 inhibitor. In some embodiments, a MRGPRX4 inhibitor can further reduce severity of or prevent a condition, disorder or disease, e.g., itch. Various MRGPRX4 inhibitors, e.g., those described in WO 2022/079245, WO 2018/232316, US 20210032213, WO 2020/198537, WO 2021/211839, and WO 2022/061008, the inhibitors and methods for identifying, characterizing, assessing, preparing and/or using the inhibitors of each of which are independently incorporated herein by reference.

Additionally or alternatively, in some embodiments, a compound is utilized in combination with a MRGPRB inhibitor. In some embodiments, a compound is utilized in combination with a MRGPRB2 inhibitor. In some embodiments, a MRGPRB2 inhibitor can further reduce severity of or prevent a condition, disorder or disease, e.g., itch. Various MRGPRB2 inhibitors, e.g., those described in WO 2016/019246, US 20200370051, and US 20170204419, the inhibitors and methods for identifying, characterizing, assessing, preparing and/or using the inhibitors of each of which are independently incorporated herein by reference.

In some embodiments, an agent, e.g., which is, comprises or delivers a GPCR inhibitor, MRGPRX inhibitor, or MRGPRB inhibitor, can further reduces severity of or prevent a condition, disorder or disease, e.g., itch. In some embodiments, such an agent is utilized at lower levels (e.g., doses, unit doses, total doses, frequency, etc.) compared to when in combination with a reference compound, e.g., a comparable compound with 3′-OH that is absent from a provided compound.

In some embodiments, the present disclosure provides a composition comprising or delivering a provided compound or a salt thereof, and an additional agent as described herein. In some embodiments, the present disclosure provides kits or packages, e.g., pharmaceutical product boxes, comprising a provided compound or a salt thereof, and an additional agent as described herein. In some embodiments, a kit or package further comprises instructions how to use compounds and/or agents therein. For example, in some embodiments, an additional agent is a phenylbutyric acid or a salt thereof. In some embodiments, an additional agent is a phenylbutyrate salt. In some embodiments, a salt is a pharmaceutically acceptable salt. In some embodiments, it is sodium phenylbutyrate.

When utilized with other agents, provided compounds may be administered or delivered concurrently with, prior to, or after such agents. In some embodiments, a compound is administered or delivered concurrently with another agent. In some embodiments, a compound is administered or delivered in the same composition as another agent. In some embodiments, a compound is administered or delivered prior to another agent. In some embodiments, a compound is administered or delivered after another agent. In some embodiments, they are administered or delivered to a subject such that such a subject is at a time point or for a time period exposed simultaneously to them. In some embodiments, at a time point or for a time period, a subject is under effects, e.g., therapeutic effects, of a provided compound and one or more other agents.

### Pharmaceutical Compositions and Administration

In some embodiments, the present disclosure provides pharmaceutical compositions that comprise a provided compound or a pharmaceutically acceptable salt thereof, and a pharmaceutically acceptable carrier. In some embodiments, the present disclosure provides pharmaceutical compositions that can deliver a provided compound or a pharmaceutically acceptable salt thereof, and a pharmaceutically acceptable carrier.

Various technologies, e.g., routes, modes, dosage regimens, etc. may be utilized to administer and/or deliver provided compounds and compositions in accordance with the present disclosure. In some embodiments, a route and/or mode of administration can vary depending upon desired results. One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to discretion of a practitioner.

In some embodiments, compounds can be incorporated into and administered as pharmaceutical compositions. Such pharmaceutical compositions are useful for, among other things, administration and delivery to a subject in vivo or ex vivo. In some embodiments, pharmaceutical compositions also contain a pharmaceutically acceptable carrier. In some embodiments, a pharmaceutically acceptable carrier is a pharmaceutical agent that does not itself induce an immune response harmful to the individual receiving a composition, and which may be administered without undue toxicity. Pharmaceutically acceptable carriers (or excipients) include, but are not limited to, liquids such as water, saline, glycerol, sugars and ethanol. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with many acids, including but not limited to, hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, diethanolamine, ethanolamine, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. In some embodiments, pharmaceutical compositions or formulations are tablets (coated or uncoated), capsules (hard or soft), microbeads, powder, granules and/or crystals. Supplementary active compounds (e.g., preservatives, antibacterial, antiviral and antifungal agents) can also be incorporated into pharmaceutical compositions.

Pharmaceutical compositions can be formulated to be compatible with a particular route of administration or delivery as set forth herein or known to one of skill in the art.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Co-solvents and adjuvants may be added to compositions and formulations. Non-limiting examples of co-solvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical compositions and delivery systems appropriate for compositions, methods and uses of the present disclosure are known in the art (see, e.g., Remington: The Science and Practice of Pharmacy. 21st Edition. Philadelphia, PA. Lippincott Williams & Wilkins, 2005) and can be utilized in accordance with the present disclosure.

In some embodiments, the present disclosure provides methods for delivering provided compounds and compositions into cells, animals or subjects. In some embodiments, such methods include contacting a subject (e.g., a cell ortissue of a subject) with, or administering or delivering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including route of administration, amount to achieve a therapeutic effect, specific condition, disorder or disease treated, any host immune response to administered compound or composition, stability of administered compound or composition, etc.

An effective amount or a sufficient amount can be provided in a single administration, may require multiple administrations, and, can be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases, etc. In some embodiments, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion or orally. In some embodiments, delivery of a pharmaceutical composition in vivo may generally be accomplished via injection using a conventional syringe, although other delivery methods such as convection-enhanced delivery can also be used (see, e.g., U.S. Pat. No. 5,720,720). In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients may determine optimal routes for administration of compounds and compositions as described herein.

### EXEMPLIFICATION

Certain examples of provided technologies (e.g., compounds, compositions, methods (methods of preparation, use, assessment, etc.), etc.) are described herein. Those skilled in the art reading the present disclosure appreciate that various technologies, including those described below and modifications, variants and derivatives thereof, are available for manufacturing, characterizing and/or assessing provided technologies in accordance with the present disclosure. For example, in some embodiments, certain useful reactions and assays are described in WO 2016073767, WO 2016086115, WO 2016086134, WO 2016086169, WO 2016086218, WO 2016130809, WO 2016161003, WO 2017147137, WO 2017147159, WO 2017147174, WO 2017189651, WO 2017189652, WO 2017189663, WO 2017201150, WO 2017201152, WO 2017201155, WO 2018067704, WO 2018081285, WO 2018102418, WO 2018152171, WO 2018187804, WO 2019118571, WO 2019160813, WO 2020231917, Yu et al. eLife 2019; 8:e48431, and can be utilized in accordance with the present disclosure.

### Example 1. Synthesis of Compound 1-1

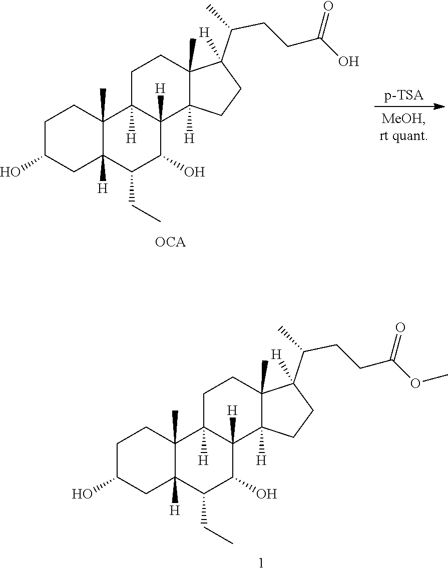

Step 1: Synthesis of compound 1. To a solution of OCA (2.5 g) in MeOH (50 mL) was added p-TSA (450 mg), and the mixture was stirred at room temperature for 2 h. Most of the solvent was removed, and the residue was dissolved in ether, washed with saturated NaHCO₃ solution. The combined organic layers were dried over anhydrous Na₂SO₄, filtered, and concentrated in vacuo to give the desired product 1 (2.64 g, quant.). 1H NMR (400 MHz, CDCl3) δ 3.70 (s, 1H), 3.66 (s, 3H), 3.40 (bs, 1H), 2.39-2.32 (m, 1H), 2.27-2.17 (m, 1H), 2.08-0.78 (m, 36H), 0.66 (s, 3H).

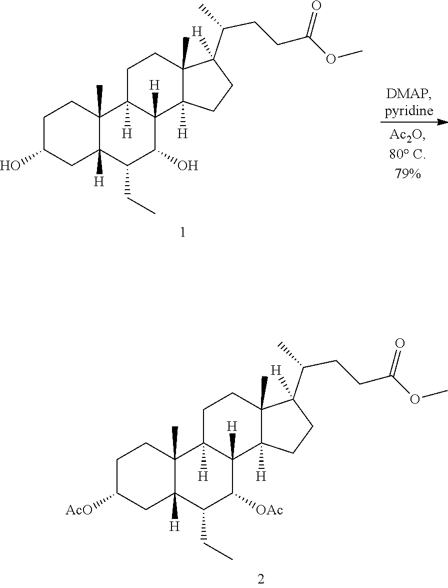

Step 2: Synthesis of compound 2. To a solution of 1 (211.8 mg) in pyridine (1 mL) was added Ac₂O (0.3 mL) and DMAP (6 mg), and the mixture was stirred at 80° C. overnight. After cooling to room temperature, solvent was removed, followed by addition of saturated NaHCO₃ solution, and the mixture was extracted with EA. The combined organic layers were dried over anhydrous Na₂SO₄, filtered, and concentrated in vacuo. The crude product was purified by silica gel chromatography to give the desired product 2 (198.0 mg, 79%). 1H NMR (400 MHz, CDCl3) δ 5.12 (d, J=21.3 Hz, 1H), 4.63-4.46 (m, 1H), 3.66 (s, 3H), 2.44-2.27 (m, 2H), 2.27-2.14 (m, 2H), 2.07 (s, 3H), 2.04 (s, 3H), 2.00-0.79 (m, 32H), 0.65 (d, J=10.6 Hz, 3H).

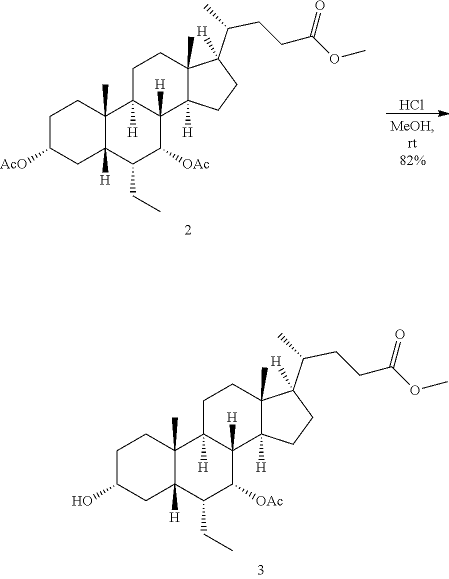

Step 3: Synthesis of compound 3. To a solution of 2 (107.5 mg) in MeOH (1 mL) was added concentrated HCl (0.2 mL), and the mixture was stirred at room temperature overnight. Solvent was removed, followed by addition of saturated NaHCO₃ solution, and the mixture was extracted with EA. The combined organic layers were dried over anhydrous Na₂SO₄, filtered, and concentrated in vacuo. The crude product was purified by silica gel chromatography to give the desired product 3 (80.6 mg, 82%).

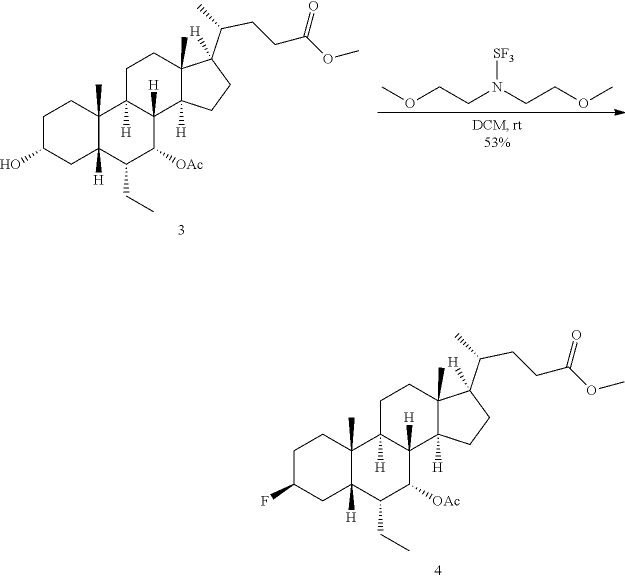

Step 4: Synthesis of compound 4. To a solution of 3 (2.3 g, 4.8 mmol) in DCM (20 mL) was added DAST (6.9 mL) at ice bath. The mixture was stirred at room temperature for 5 h. After TLC indicated complete reaction, the solvent is evaporated under reduced pressure, followed by addition of saturated NaHCO₃ solution, and the mixture was extracted with EA. The combined organic layers were dried over anhydrous Na₂SO₄, filtered, and concentrated under reduced pressure. The crude product was purified by silica gel chromatography to give the desired product 4 (1.21 g, 53% yield). LC-MS, ES⁻ (m/z): 477.35 (M-1). ¹H NMR (400 MHz, CDCl₃) δ 5.09 (s, 1H), 4.95-4.72 (d, 1H), 3.64 (s, 3H), 2.39-2.28 (m, 1H), 2.25-2.15 (m, 1H), 2.04-2.02 (d, 3H), 1.99-1.91 (m, 1H), 1.86-1.59 (m, 8H), 1.52-1.37 (m, 4H), 1.36-1.19 (m, 5H), 1.17-0.99 (m, 6H), 0.95 (s, 3H), 0.92-0.85 (m, 7H), 0.63 (s, 3H).

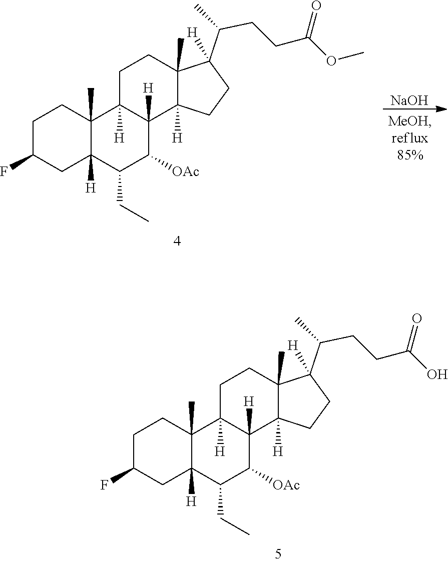

Step 5: Synthesis of compound 5. To a solution of 4 (18.0 mg) in MeOH (1 mL) was added NaOH (15 mg), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 5 (14.9 mg, 85%).

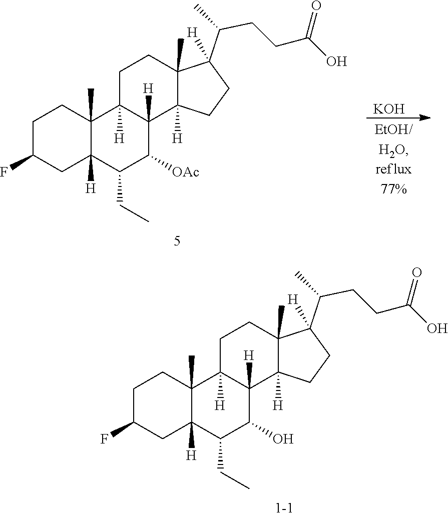

Step 6: Synthesis of compound 1-1. To a solution of 5 (3.0 mg) in EtOH (0.5 mL) was added KOH (100 mg) in water (0.5 mL), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 1-1 (2.1 mg, 77%). LC-MS, ES⁻ (m/z): 421.32 (M-1). ¹H NMR (400 MHz, CDCl₃) δ 4.92-4.70 (d, 1H), 3.72 (s, 1H), 2.44-2.34 (m, 1H), 2.30-2.19 (m, 1H), 2.01-1.74 (d, 5H), 1.73-1.57 (m, 5H), 1.56-1.53 (m, 1H), 1.51-1.28 (m, 10H), 1.25-1.22 (m, 2H), 1.20-1.09 (m, 3H), 0.95-0.84 (m, 9H), 0.66 (s, 3H).

Alternative route for compound 1-1:

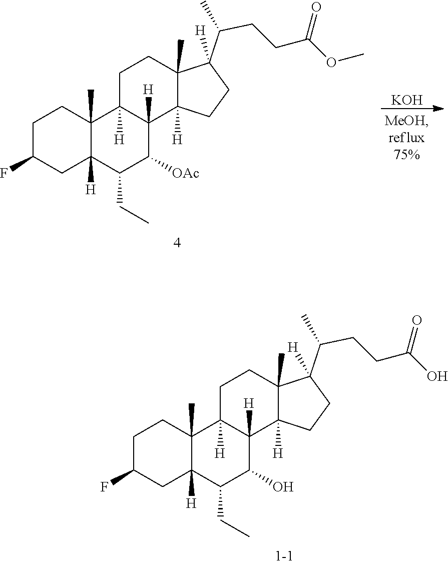

To a solution of 4 (456.0 mg, 0.95 mmol) in CH₃OH (60 mL) was added KOH (15.82 g, 323 mmol), and the mixture was refluxed overnight. After cooling to room temperature, the reaction mixture was acidified with 4M HCl. Then the solvent was removed, and the residue was extracted with EA. The combined organic layers were dried over anhydrous Na₂SO₄, filtered, and concentrated in vacuum. The crude product was purified by silica gel chromatography to give the desired product 1-1 (301.0 mg, 75% yield). LC-MS, ES⁻ (m/z): 421.32 (M-1). ¹H NMR (400 MHz, CDCl₃) δ 4.92-4.70 (d, 1H), 3.72 (s, 1H), 2.44-2.34 (m, 1H), 2.30-2.19 (m, 1H), 2.01-1.74 (d, 5H), 1.73-1.57 (m, 5H), 1.56-1.53 (m, 1H), 1.51-1.28 (m, 10H), 1.25-1.22 (m, 2H), 1.20-1.09 (m, 3H), 0.95-0.84 (m, 9H), 0.66 (s, 3H).

### Example 2. Synthesis of Compound 1-2

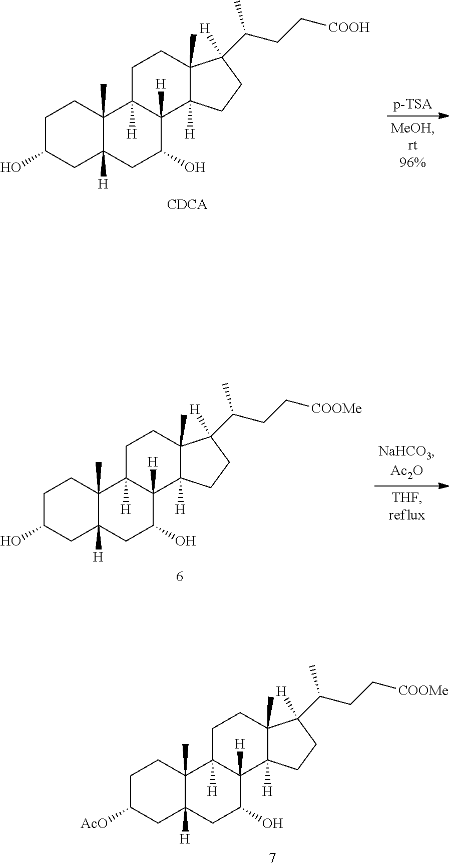

Step 1: Synthesis of compound 6. To a solution of CDCA (1.5 g) in MeOH (30 mL) was added p-TSA (300 mg), and the mixture was stirred at room temperature for 6 h. Most of the solvent was removed, and the residue was dissolved in ether, washed with saturated NaHCO₃ solution. The combined organic layers were dried over anhydrous Na₂SO₄, filtered, and concentrated in vacuo to give the desired product 6 (1.49 g, 96%).
Step 2: Synthesis of compound 7. To a solution of 6 (1.3 g) in THF (25 mL) was added Ac₂O (5.4 mL) and NaHCO₃ (5.4 g), and the mixture was refluxed overnight. After cooling to room temperature, water was added, and the mixture was extracted with EA. The combined organic layers were dried over anhydrous Na₂SO₄, filtered, and concentrated in vacuo to give the desired product 7 (1.9 g), which was used in the next step without further purification.

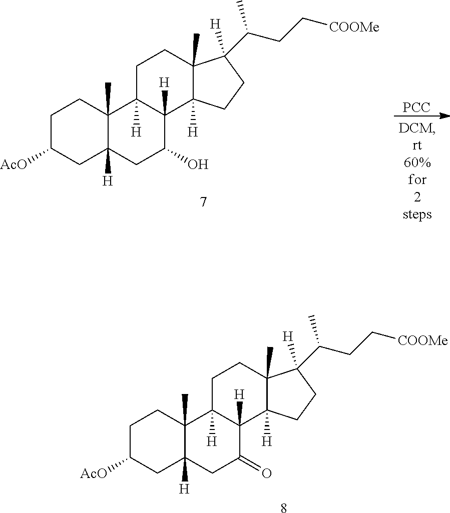

Step 3: Synthesis of compound 8. To a solution of 7 (1.9 g) in DCM (40 mL) was added PCC (2.7 g), and the mixture was stirred at room temperature overnight. Silica gel (10 g) was added, and the mixture was concentrated. The crude product was purified by silica gel chromatography to give the desired product 8 (850 mg, 60% for 2 steps).

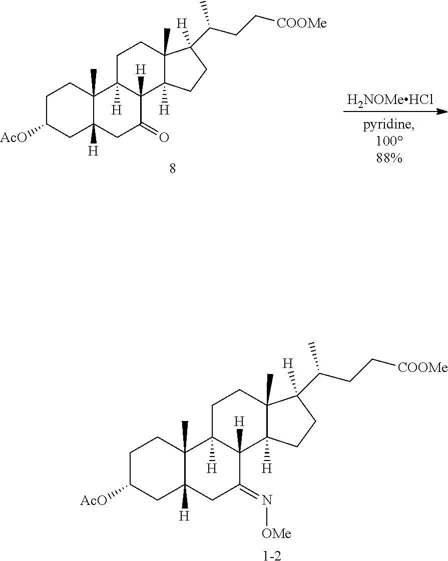

Step 4: Synthesis of compound 1-2. A mixture of 8 (76.1 mg) and O-methylhydroxylamine hydrochloride (28.5 mg) in pyridine (1 mL) was heated at 100° C. for 5 h. After cooling to room temperature, solvent was removed. EA was added to the residue, and the mixture was washed with water, 0.01 M HCl, and brine. The combined organic layers were dried over anhydrous Na₂SO₄, filtered, and concentrated in vacuo. The crude product was purified by silica gel chromatography to give the desired product 1-2 (71.3 mg, 88%). ¹H NMR (400 MHz, Chloroform-d) δ 4.73-4.65 (m, 1H), 3.78 (s, 3H), 3.66 (s, 3H), 2.96 (d, J=13.0, 1H), 0.93 (dd, J=6.4, 1.5 Hz, 3H), 0.66 (s, 3H).

### Example 3. Synthesis of Compound 1-3

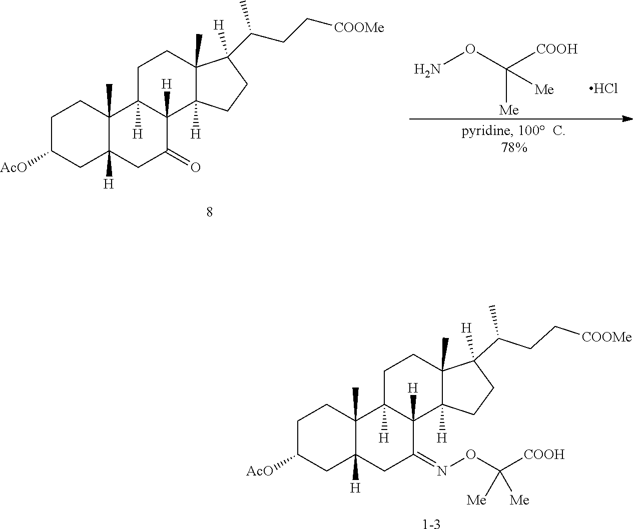

A mixture of 8 (431.0 mg) and 2-(aminooxy)-2-methylpropanoic acid hydrochloride (180.3 mg) in pyridine (3 mL) was heated at 100° C. for 5 h. After cooling to room temperature, solvent was removed. EA was added to the residue, and the mixture was washed with water, 0.01 M HCl, and brine. The combined organic layers were dried over anhydrous Na₂SO₄, filtered, and concentrated in vacuo. The crude product was purified by silica gel chromatography to give the desired product 1-3 (412.7 mg, 78%).

¹H NMR (400 MHz, Chloroform-d) δ 10.81 (s, 1H), 4.68 (s, 1H), 3.67 (s, 3H), 3.04 (dd, J=13.0, 2.2 Hz, 1H), 0.93 (d, J=6.4 Hz, 4H), 0.67 (s, 3H).

### Example 4. Synthesis of Compound 1-5

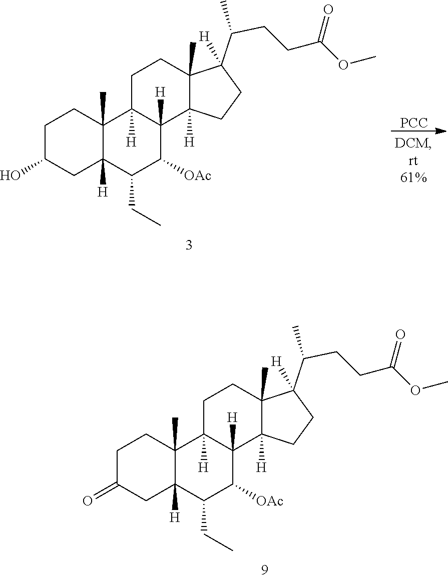

Step 1: Synthesis of compound 9. To a solution of 3 (11.3 g, 23.6 mmol) in DCM (400 mL) was added silica gel (21 g) and PCC (10.3 g, 47.3 mmol), and the mixture was stirred at room temperature overnight. The mixture was concentrated, and the crude product was purified by silica gel chromatography (PE:EA=10:1˜5:1) to give the desired product 9 (6.86 g, 61%). ¹H NMR (400 MHz, CDCl₃): δ 5.17 (s, 1H), 3.66 36 (s, 3H), 2.30-2.45 (m, 2H), 2.15-2.25 (m, 3H), 0.70-1.90 (m, 34H), 0.68 (s, 3H).

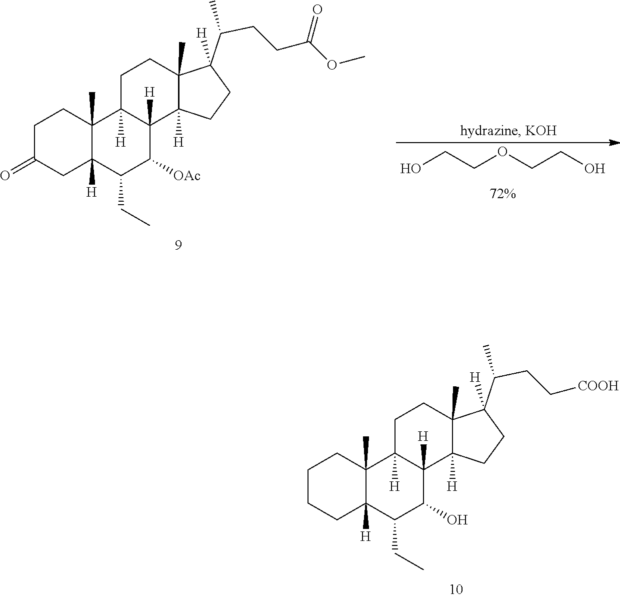

Step 2: Synthesis of compound 10. To a solution of 9 (600 mg) in 2,2′-oxybis(ethan-1-ol) (20 mL) was added KOH (2.3 g) and hydrazine (2.5 mL), and the mixture was stirred at 120° C. for 2 h, followed by 180° C. overnight. The mixture was cooled to room temperature, quenched with water, acidified by 1 M HCl, and extracted with EA. The combined organic layers were dried over anhydrous Na₂SO₄, filtered, and concentrated in vacuo. The crude product was purified by silica gel chromatography to give the desired product 10 (368 mg, 72%). ¹HNMR (400 MHz, CDCl₃) δ 3.69 (s, 1H), 2.36-2.44 (m, 1H), 2.20-2.30 (m, 1H), 2.10 (s, 1H), 0.82-2.00 (m, 37H), 0.66 (s, 3H).

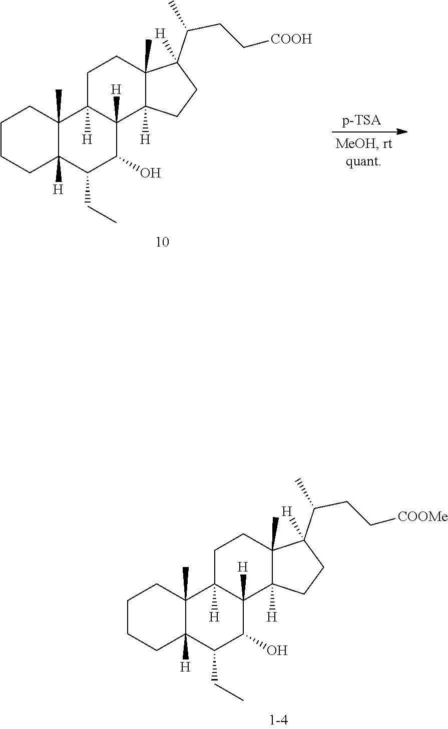

Step 3: Synthesis of compound 1-4. To a solution of 10 (110 mg) in MeOH (3 mL) was added p-TSA (21 mg), and the mixture was stirred at room temperature for 2 h. Most of the solvent was removed, and the residue was dissolved in ether, washed with saturated NaHCO₃ solution. The combined organic layers were dried over anhydrous Na₂SO₄, filtered, and concentrated in vacuo to give the desired product 1-4 (118 mg, quant.). LC-MS, ES⁺ (m/z): 419.38 (M+1).

### Example 5. Synthesis of Compound 1-5

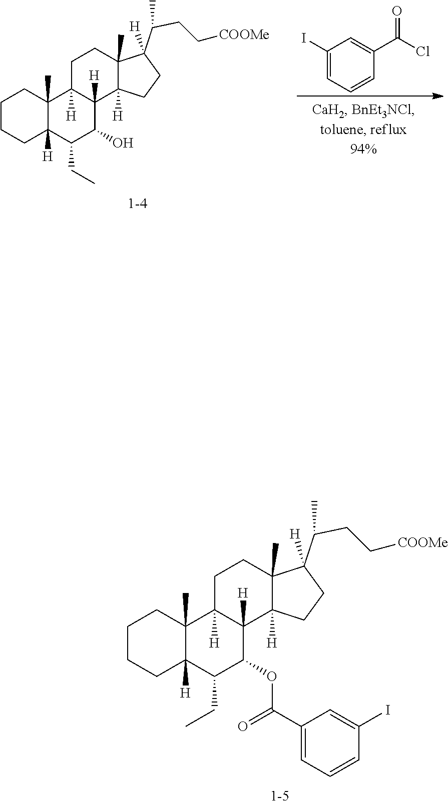

To a solution of 1-4 (550 mg) in toluene (4 mL) was added 3-iodobenzoyl chloride (0.73 mL), CaH₂ (220 mg), and BnEt₃NCl (75 mg). The mixture was refluxed for 36 h. After cooling to room temperature, most of the solvent was removed, followed by the addition of chloroform. The mixture was filtered through a pad of celite, and the filtrate was washed with saturated NaHCO₃ solution, water and brine. The combined organic layers were dried over anhydrous Na₂SO₄, filtered, and concentrated in vacuo. The crude product was purified by silica gel chromatography to give the desired product 1-5 (804 mg, 94%).

¹H NMR (400 MHz, Chloroform-d) δ 8.40 (t, J=1.7 Hz, 1H), 7.98 (dt, J=7.8, 1.3 Hz, 1H), 7.92-7.88 (m, 1H), 7.22 (t, J=7.9 Hz, 1H), 5.37 (t, J=3.0 Hz, 1H), 3.63 (s, 3H), 0.97 (s, 3H), 0.66 (s, 3H).

### Example 6. Synthesis of Compound 1-6

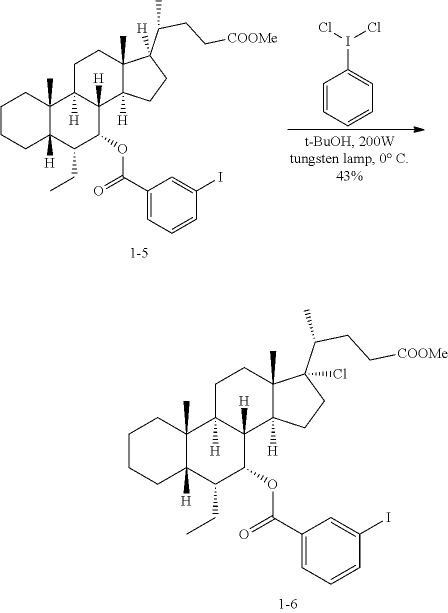

To a solution of 1-5 (90 mg) in DCM (4.5 mL) was added t-BuOH (123 μL) and dichloroiodinanylbenzene (46 mg). The mixture was bubbled with Argon for 15 min. The mixture was cooled to 0° C. and exposed to 200 W tungsten lamp for 1 h. Solvent was removed, and the crude product was purified by silica gel chromatography to give the desired product 1-6 (38.5 mg, 43%). ¹H NMR (400 MHz, Chloroform-d) δ 8.39 (t, J=1.7 Hz, 1H), 7.98 (dt, J=7.8, 1.4 Hz, 1H), 7.88 (dt, J=8.1, 1.3 Hz, 1H), 7.21 (t, J=7.9 Hz, 1H), 5.34 (t, J=3.0 Hz, 1H), 3.65 (s, 3H), 0.98 (d, J=6.3 Hz, 6H), 0.82 (s, 3H).

### Example 7. Synthesis of Compound 1-7

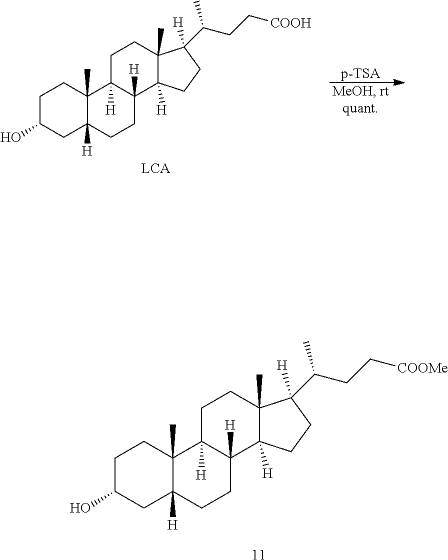

Step 1: Synthesis of compound 11. To a solution of LCA (1 g) in MeOH (20 mL) was added p-TSA (100 mg), and the mixture was stirred at room temperature overnight. Most of the solvent was removed, and the residue was dissolved in DCM, washed with saturated NaHCO₃ solution. The combined organic layers were dried over anhydrous Na₂SO₄, filtered, and concentrated in vacuo to give the desired product 11 (1.06 g, quant.).

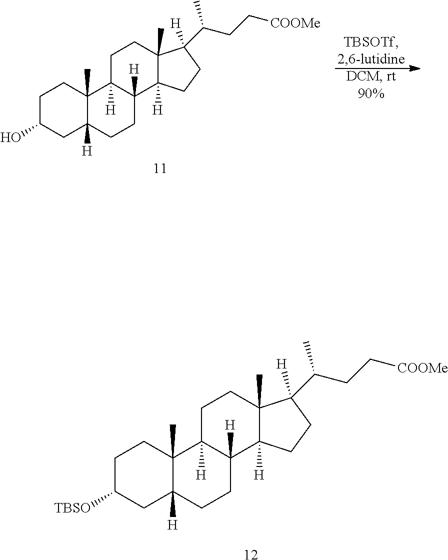

Step 2: Synthesis of compound 12. To a solution of compound 11 (200 mg) in DCM (10 mL) at 0° C. was added 2,6-lutidine (0.6 mL) and TBSOTf (0.35 mL), and the mixture was stirred at room temperature for 2 h. The mixture was concentrated, and the crude product was purified by silica gel chromatography to give the desired product 12 (232.6 mg, 90%).

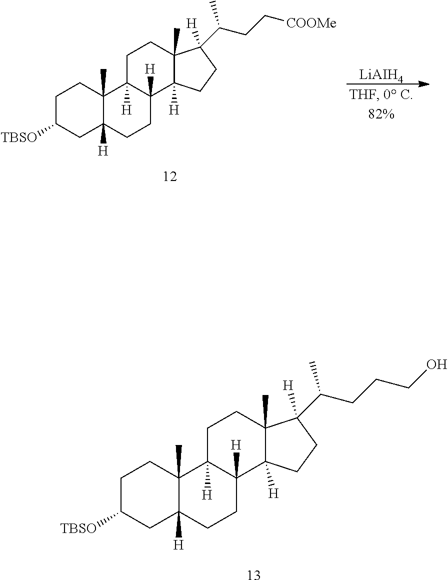

Step 3: Synthesis of compound 13. To a solution of 12 (23.26 g) in THF (100 mL) at 0° C. was added LiAlH₄ (5 g), and the mixture was stirred at 0° C. for 2 h. The mixture was concentrated, and the crude product was purified by silica gel chromatography to give the desired product 13 (18.01 g, 82%).

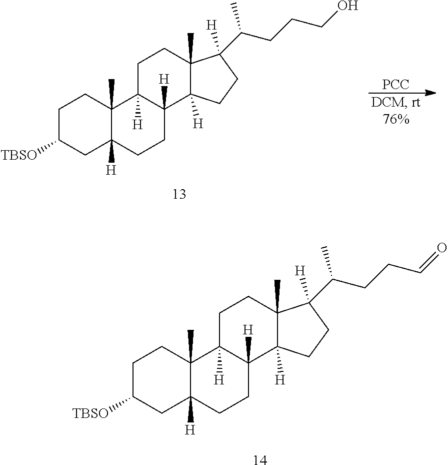

Step 4: Synthesis of compound 14. To a solution of 13 (11.31 g) in DCM (100 mL) was added PCC (2.2 g), and the mixture was stirred at room temperature overnight. The mixture was concentrated, and the crude product was purified by silica gel chromatography to give the desired product 14 (8.56 mg, 76%).

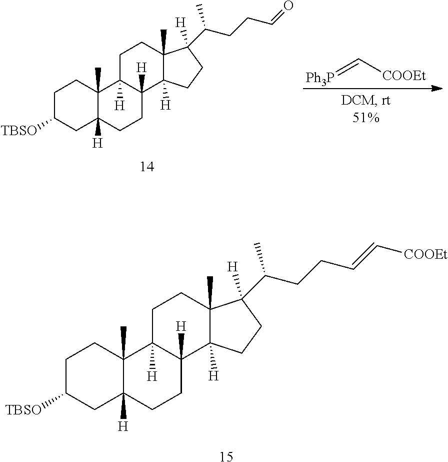

Step 5: Synthesis of compound 15. To a solution of 14 (5.37 g) in DCM (20 mL) was added Wittig reagent (5.8 g), and the mixture was stirred at room temperature for 12 h. The mixture was concentrated, and the crude product was purified by silica gel chromatography to give the desired product 15 (3.17 g, 51%).

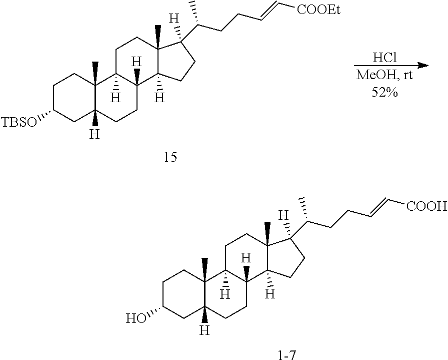

Step 6: Synthesis of compound 1-7. To a solution of 15 (22.4 mg) in MeOH (2 mL) was added concentrated HCl (4 drops), and the mixture was stirred at room temperature overnight. Solvent was removed, followed by addition of saturated NaHCO₃ solution, and the mixture was extracted with EA. The combined organic layers were dried over anhydrous Na₂SO₄, filtered, and concentrated in vacuo. The crude product was purified by silica gel chromatography to give the desired product 1-7 (8.6 mg, 52%). ¹H NMR (400 MHz, Chloroform-d) δ 7.06 (dt, J=14.7, 6.9 Hz, 1H), 5.83 (d, J=15.6 Hz, 1H), 3.67-3.58 (m, 1H), 0.93 (d, J=7.4 Hz, 6H), 0.64 (s, 3H).

### Example 8. Synthesis of Compound 2-1

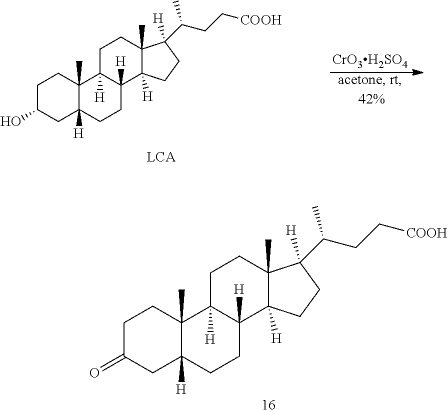

Step 1: Synthesis ot compound 16. 1o a solution of LCA (96 mg) in acetone (10 mL) at U C was added CrO₃·H₂SO₄ (2M, 250 μL) dropwise, and the mixture was stirred at room temperature for 2 h. Saturated NaHCO₃ solution was added, and the mixture was extracted with EA. The combined organic layers were dried over anhydrous Na₂SO₄, filtered, and concentrated in vacuo. The crude product was purified by silica gel chromatography to give the desired product 16 (42.5 mg, 42%). ¹H NMR (400 MHz, Chloroform-d) δ 2.69 (t, J=14.2 Hz, 1H), 1.01 (s, 3H), 0.93 (d, J=6.4 Hz, 3H), 0.68 (s, 3H).

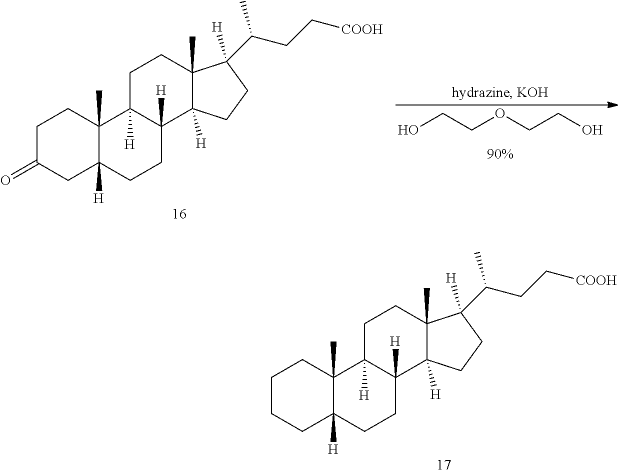

Step 2: Synthesis of compound 17. To a solution of 16 (20.7 mg) in 2, 2′-oxybis(ethan-1-ol) (1 mL) was added KOH (94 mg) and hydrazine (100 μL), and the mixture was stirred at 110° C. for 2 h, followed by 180° C. for 4 h. The mixture was cooled to room temperature, quenched with water, acidified by 1 M HCl, and extracted with EA. The combined organic layers were dried over anhydrous Na₂SO₄, filtered, and concentrated in vacuo. The crude product was purified by silica gel chromatography to give the desired product 17 (17.9 mg, 90%). ¹H NMR (400 MHz, Chloroform-d) (2.44-2.35 (m, 1H), 2.30-2.22 (m, 1H), 0.94-0.90 (m, 6H), 0.65 (s, 3H).

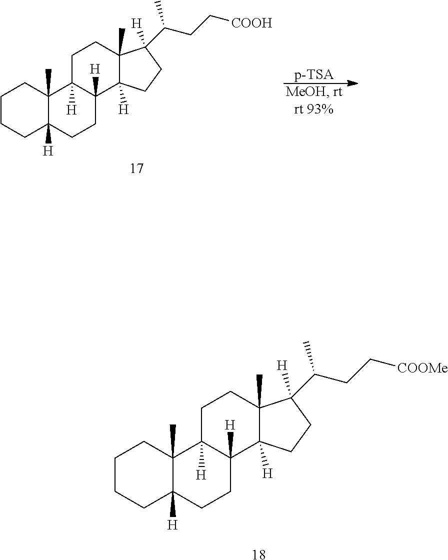

Step 3: Synthesis of compound 18. To a solution of 17 (25.6 mg) in MeOH (1 mL) was added p-TSA (20 mg), and the mixture was stirred at room temperature overnight. Most of the solvent was removed, and the residue was dissolved in ether, washed with saturated NaHCO₃ solution. The combined organic layers were dried over anhydrous Na₂SO₄, filtered, and concentrated in vacuo. The crude product was purified by silica gel chromatography to give the desired product 18 (24.8 mg, 93%).

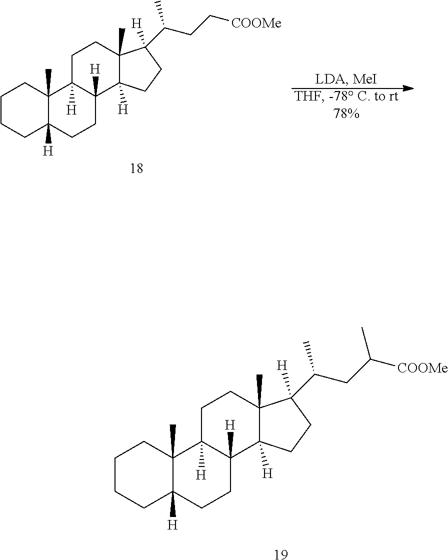

Step 4: Synthesis of compound 19. A solution of DIPA (24 μL) in dry THF (1.5 mL) was cooled to −78° C., and n-BuLi (100 μL) was added. The mixture was stirred at −78° C. for 30 min, followed by addition of a solution of 18 (24.8 mg) in THF (1 mL), and the mixture was stirred at −78° C. for 20 min, Then Mel (29 μL) was added, and the mixture was stirred at room temperature for another 8 h. The mixture was quenched with water, and extracted with EA. The combined organic layers were dried over anhydrous Na₂SO₄, filtered, and concentrated in vacuo. The crude product was purified by silica gel chromatography to give the desired product 19 (20.0 mg, 78%).

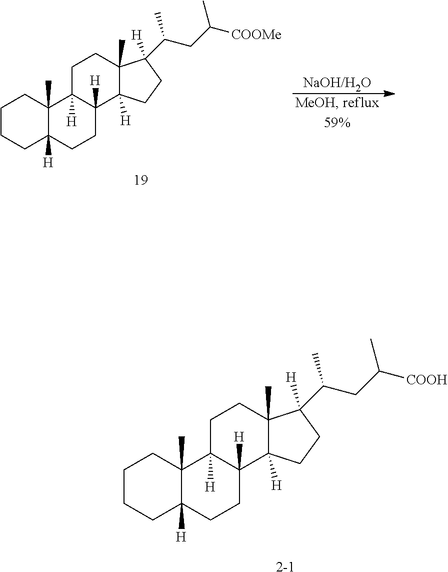

Step 5: Synthesis of compound 2-1. To a solution of 19 (20.0 mg) in MeOH (0.5 mL) was added NaOH (21.3 mg) in water (0.5 mL), and the mixture was refluxed for 4 h. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 2-1 (11.4 mg, 59%). ¹H NMR (400 MHz, Chloroform-d) δ 2.64-2.56 (m, 1H), 0.66 (s, 2H), 0.63 (s, 3H).

### Example 9. Synthesis of Compound 2-2

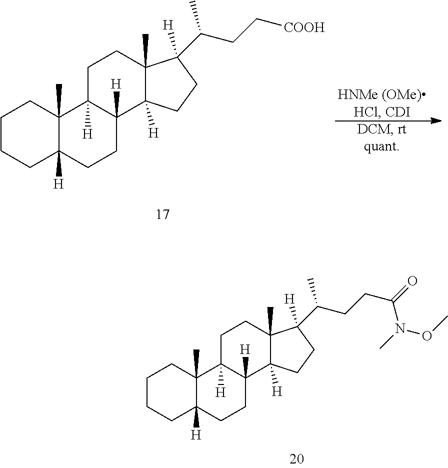

Step 1: Synthesis of compound 20. To a solution of 17 (20.0 mg) in DCM (1 mL) was added MeNH(OMe)·HCl (8.0 mg) and CDI (14.0 mg), and the mixture was stirred at room temperature for 6 h. The mixture was quenched with 1 M NaOH to adjust pH to 10. The organic layer was separated, concentrated, and the crude product was purified by silica gel chromatography to give the desired product 20 (25.0 mg, quant.).

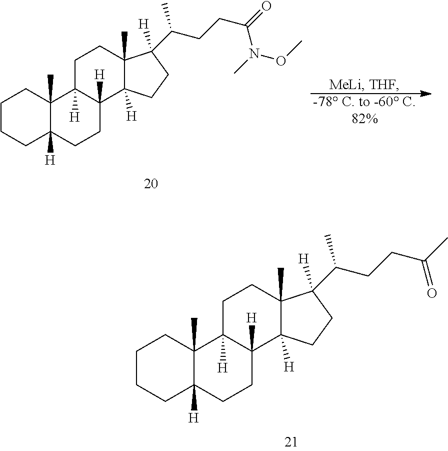

Step 2: Synthesis of compound 21. To a solution of 20 (60.3 mg) in THF (2 mL) at −78° C. was added MeLi (0.9 mL, 1 M in THF), and the mixture was stirred at −60° C. for 2 h. The mixture was quenched with water, and extracted with EA. The combined organic layers were dried over anhydrous Na₂SO₄, filtered, and concentrated in vacuo. The crude product was purified by silica gel chromatography to give the desired product 21 (44.2 mg, 82%). ¹H NMR (400 MHz, Chloroform-d) δ 3.17 (s, 3H), 2.50-2.38 (m, 1H), 2.36-2.27 (m, 1H), 0.94 (d, J=6.5 Hz, 3H), 0.91 (s, 3H), 0.65 (s, 3H).

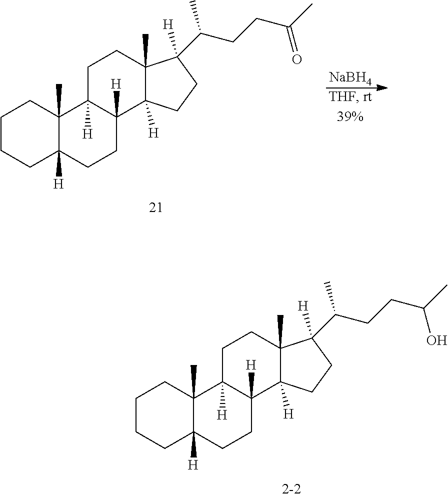

Step 3: Synthesis of compound 2-2. To a solution of 21 (8.5 mg) in THF (1 mL) was added NaBH₄ (4.0 mg), and the mixture was stirred at room temperature for 3 h. The mixture was concentrated, and the crude product was purified by silica gel chromatography to give the desired product 2-2 (3.3 mg, 39%). ¹H NMR (400 MHz, Chloroform-d) δ 3.78-3.70 (m, 1H), 0.64 (s, 3H).

### Example 10. Synthesis of Compound 2-3

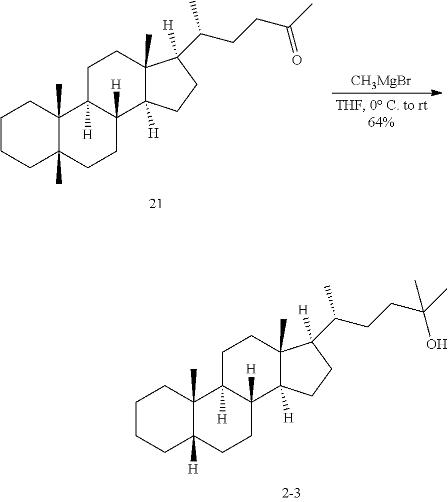

To a solution of 21 (9.1 mg) in THF (1 mL) at 0° C. was added CH₃MgBr (19 μL, 3 M in ether), and the mixture was stirred at room temperature for 3 h. The mixture was concentrated, and the crude product was purified by silica gel chromatography to give the desired product 2-3 (6.1 mg, 64%). ¹H NMR (400 MHz, Chloroform-d) δ 3.71 (s, 1H), 0.65 (d, J=5.4 Hz, 6H).

### Example 11. Synthesis of Compound 2-4

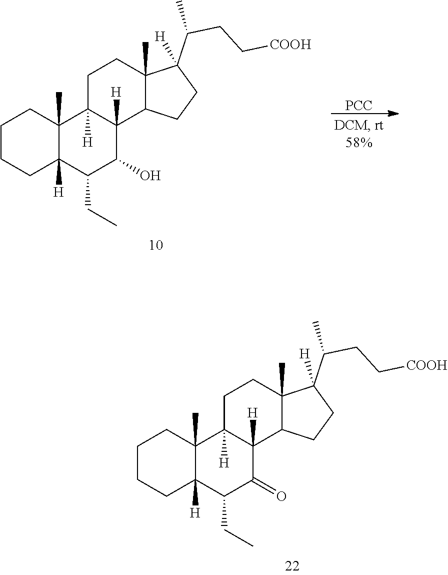

Step 1: Synthesis of compound 22. To a solution of 10 (111.1 mg) in DCM (5 mL) was added silica gel (300 mg) and PCC (85.8 mg), and the mixture was stirred at room for 1.5 h. The mixture was concentrated, and the crude product was purified by silica gel chromatography to give the desired product 22 (64.4 mg, 58%).

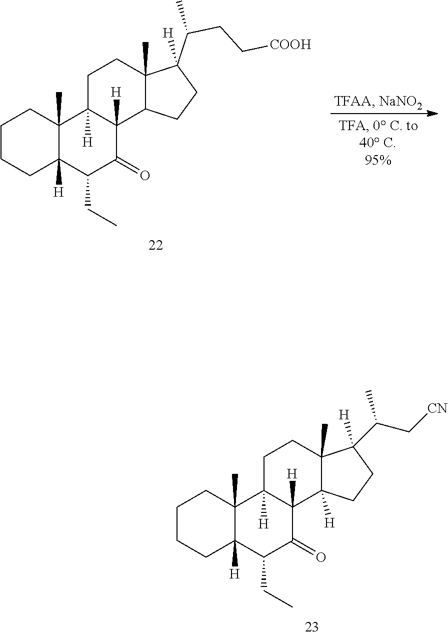

Step 2: Synthesis of compound 23. An ice-cooling mixture of 22 (39.1 mg), TFA (0.35 mL) and TFAA (90 μL) was stirred until the solution become clear, and then NaNO₂ (21 mg) was added in small portions at 0° C. The resulted mixture was stirred at 0° C. for 1 h, and then the mixture was warmed to 40° C., and stirred for 2 h. After cooling to room temperature, the mixture was neutralized with 1M NaOH solution, and extracted with EA. The combined organic layers were dried over anhydrous Na₂SO₄, filtered, and concentrated in vacuo. The crude product was purified by silica gel chromatography to give the desired product 23 (34.1 mg, 95%).

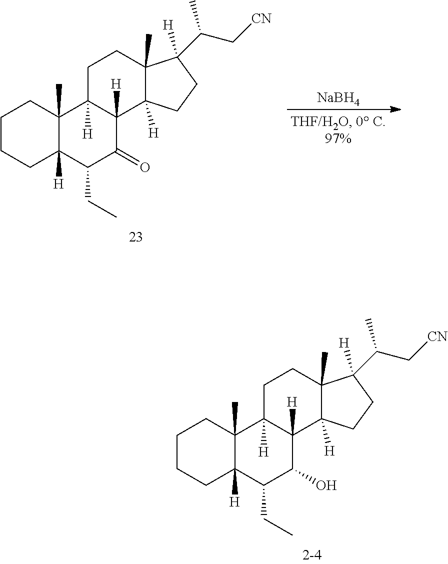

Step 3: Synthesis of compound 2-4. To a solution of 23 (24.3 mg) in THF/H₂O (4:1, 2 mL) at 0° C. was added NaBH₄ (15.1 mg), and the mixture was stirred at 0° C. for 2 h. The mixture was quenched with 1M HCl solution, and extracted with EA. The combined organic layers were dried over anhydrous Na₂SO₄, filtered, and concentrated in vacuo. The crude product was purified by silica gel chromatography to give the desired product 2-4 (23.7 mg, 97%). ¹H NMR (400 MHz, Chloroform-d) δ 4.98-3.98 (m, 2H), 3.68 (s, 1H), 0.93-0.86 (m, 9H), 0.64 (s, 3H).

### Example 12. Synthesis of Compound 2-5

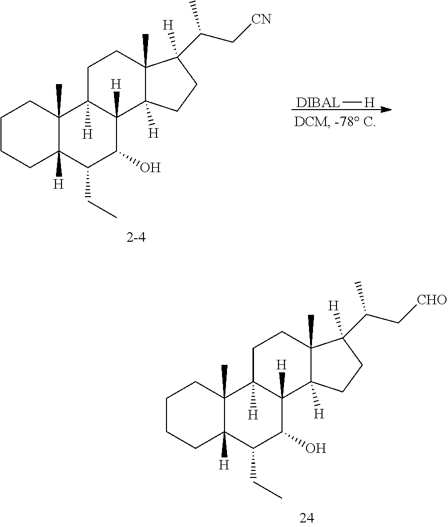

Step 1: Synthesis of compound 24. To a solution of 2-4 (4.1 mg) in DCM (0.8 mL) at −78° C. was added DIBAL-H (100 μL), and the mixture was stirred at −78° C. for 3 h. The reaction was quenched with MeOH, followed by addition of 1 M HCl. Then the mixture was extracted with DCM. The combined organic layers were dried over anhydrous Na₂SO₄, filtered, and concentrated in vacuo to give the desired product 24 (2.1 mg), which is directly used in the next step without further purification.

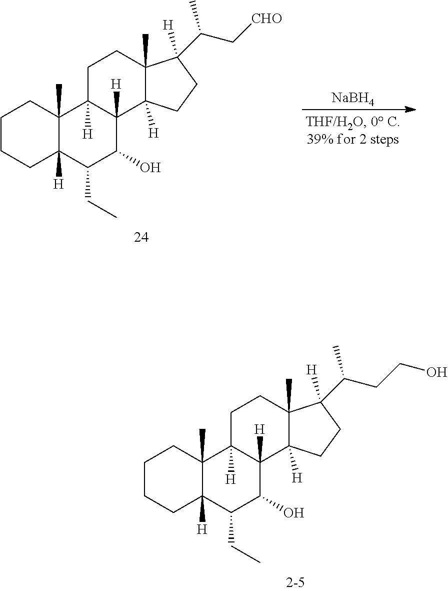

Step 2: Synthesis of compound 2-5. To a solution of 24 (2.1 mg) in THF/H₂O (4:1, 1 mL) at 0° C. was added NaBH₄ (5.0 mg), and the mixture was stirred at 0° C. for 3 h. The mixture was quenched with 1M HCl solution, and extracted with EA. The combined organic layers were dried over anhydrous Na₂SO₄, filtered, and concentrated in vacuo. The crude product was purified by silica gel chromatography to give the desired product 2-5 (1.6 mg, 39% for 2 steps). ¹H NMR (400 MHz, Chloroform-d) δ 5.34 (t, J=4.8 Hz, 1H), 3.70 (s, 2H), 0.96 (d, J=6.6 Hz, 3H), 0.92-0.86 (m, 9H), 0.67 (s, 3H).

### Example 13. Synthesis of Compound 2-6

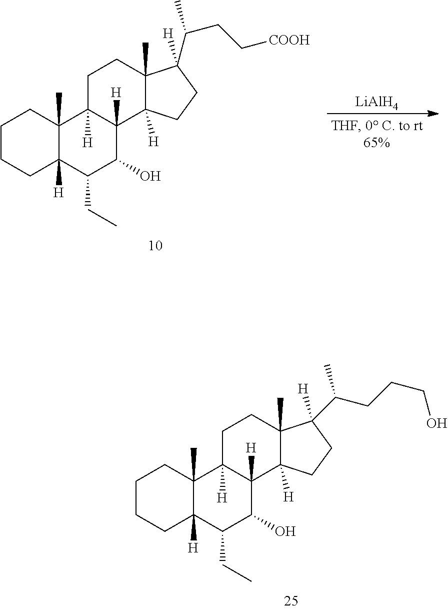

Step 1: Synthesis of compound 25. To a solution of LiAlH₄ (27.0 mg) in THF (0.5 mL) at 0° C. was added a solution of 10 (48.0 mg) in THF (0.5 mL), and the mixture was stirred at room temperature for 8 h. The mixture was quenched with 1M HCl solution, and extracted with EA. The combined organic layers were dried over anhydrous Na₂SO₄, filtered, and concentrated in vacuo. The crude product was purified by silica gel chromatography to give the desired product 25 (30.1 mg, 65%).

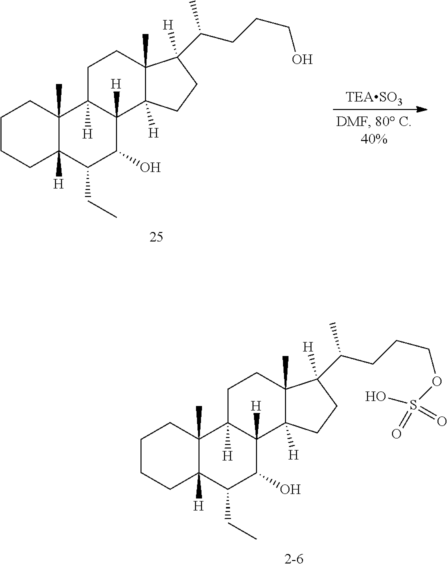

Step 2: Synthesis of compound 2-6. To a solution of 25 (17.5 mg) in DMF (1 mL) was added TEA·SO₃ (39.5 mg), and the mixture was stirred at 80° C. for 24 h. Solvent was removed, and the mixture was diluted with 1M HCl solution, and extracted with EA. The combined organic layers were dried over anhydrous Na₂SO₄, filtered, and concentrated in vacuo. The crude product was purified by silica gel chromatography to give the desired product 2-6 (8.4 mg, 40%). ¹H NMR (400 MHz, Methanol-d₄) δ 3.66 (s, 1H), 0.93-0.88 (m, 9H), 0.71 (s, 3H).

### Example 14. Synthesis of Compound 3-1

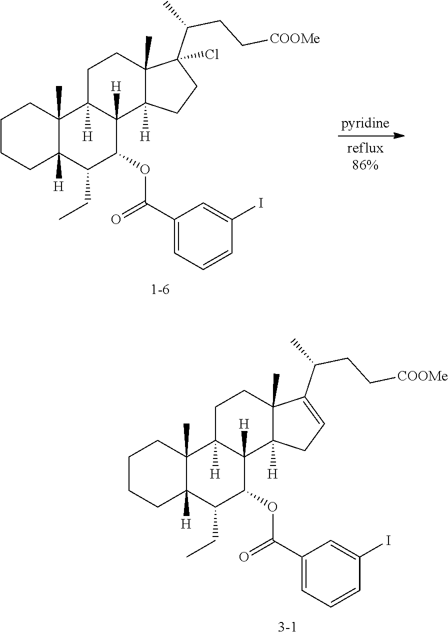

A solution of 1-6 (768 mg) in pyridine (11 mL) was refluxed overnight. After cooling to room temperature, solvent was removed, and the residue was diluted with EA. The mixture was washed with 1 M HCl, water and brine. The combined organic layers were dried over anhydrous Na₂SO₄, filtered, and concentrated in vacuo. The crude product was purified by silica gel chromatography to give the desired product 3-1 (627 mg, 86%). ¹H NMR (400 MHz, Chloroform-d) δ 8.40 (t, J=1.7 Hz, 1H), 7.97 (dt, J=7.8, 1.3 Hz, 1H), 7.88 (dt, J=8.0, 1.3 Hz, 1H), 7.20 (t, J=7.8 Hz, 1H), 5.46 (t, J=2.9 Hz, 1H), 5.18 (s, 1H), 3.64 (s, 3H), 0.89 (d, J=7.4 Hz, 3H), 0.74 (s, 3H).

### Example 15. Synthesis of Compound 3-2

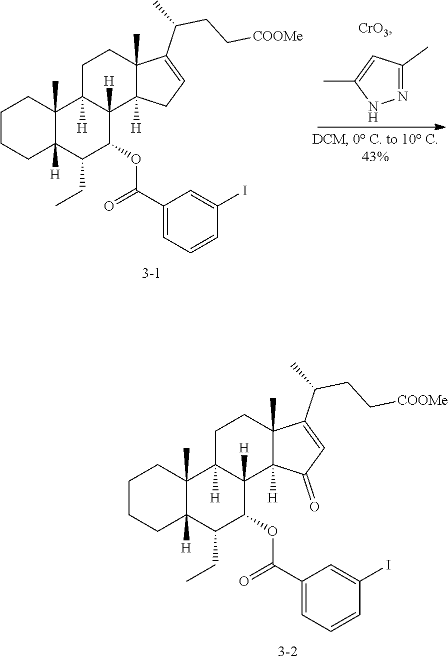

To a suspension of activated CrO₃ powder (600 mg) in DCM (6 mL) at 0° C. was added 3,5-dimethyl-1H-pyrazole (612 mg), and the mixture was stirred at 0° C. for 1 h. A solution of 3-1 (195 mg) in DCM (6 mL) was then added, and the mixture was stirred at 40° C. for 15 h. Solvent was removed, and the crude product was purified by silica gel chromatography to give the desired product 3-2 (70.6 mg, 43%). ¹H NMR (400 MHz, Chloroform-d) δ 8.38 (s, 1H), 7.94 (d, J=7.8 Hz, 1H), 7.85 (d, J=8.1 Hz, 1H), 7.17 (t, J=7.8 Hz, 1H), 6.22 (s, 1H), 5.53 (s, 1H), 3.65 (s, 3H), 2.45 (q, J=6.9 Hz, 1H), 1.10 (d, J=6.9 Hz, 3H), 1.01 (d, J=11.0 Hz, 6H), 0.93 (t, J=7.3 Hz, 3H).

### Example 16. Synthesis of Compound 3-3

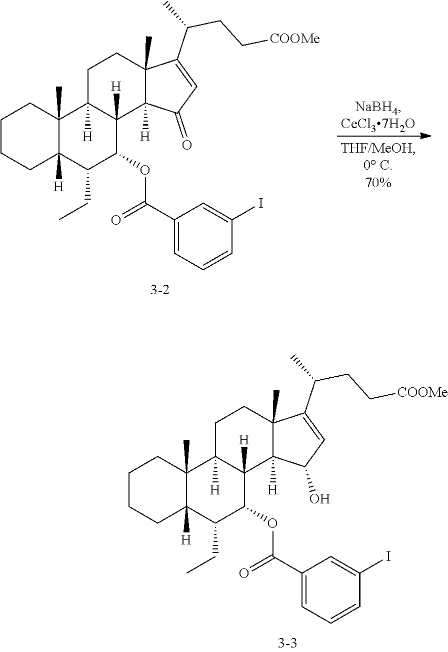

To a solution of 3-2 (70 mg) in MeOH/THF (1:2, 4.5 mL) at 0° C. was added CeCl₃·7H₂O (40 mg), and the mixture was stirred at 0° C. for 1 h. NaBH₄ (9 mg) was then added, and the mixture was stirred at 0° C. for 2 h. Then the mixture was quenched with water, and most of the solvent was removed. The residue was extracted with EA, and the combined organic layers were dried over anhydrous Na₂SO₄, filtered, and concentrated in vacuo. The crude product was purified by silica gel chromatography to give the desired product 3-3 (49 mg, 70%). ¹H NMR (400 MHz, Chloroform-d) δ 8.38 (s, 1H), 7.94 (d, J=7.8 Hz, 1H), 7.85 (d, J=8.1 Hz, 1H), 7.17 (t, J=7.8 Hz, 1H), 5.53 (s, 1H), 5.18 (s, 1H), 3.65 (s, 3H).

### Example 17. Synthesis of Compound 4-1

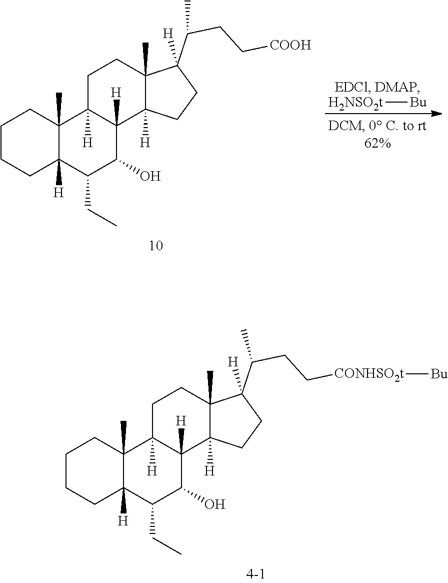

To a solution of EDCI (6 mg) in DCM (1 mL) was added DMAP (7 mg), and the mixture was stirred at room temperature for 30 min. The mixture was cooled to 0° C., followed by addition of a solution of H₂NSO₂t-Bu (3.2 mg) and compound 10 (10.0 mg) in DCM (1 mL), and the mixture was stirred at room temperature overnight. Then the mixture was quenched with saturated NaHCO₃ solution, extracted with EA, and the combined organic layers were dried over anhydrous Na₂SO₄, filtered, and concentrated in vacuo. The crude product was purified by silica gel chromatography to give the desired product 4-1 (8.0 mg, 62%). ¹H NMR (400 MHz, Chloroform-d) δ 3.69 (s, 1H), 1.48 (s, 9H), 0.94-0.88 (m, 9H), 0.66 (s, 3H).

### Example 18. Synthesis of Compound 4-2

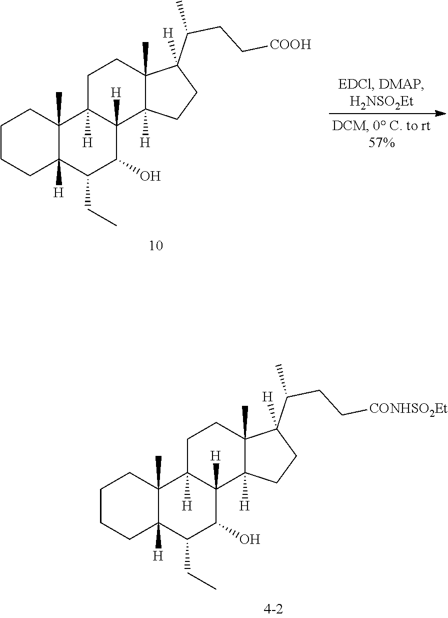

To a solution of EDCI (6 mg) in DCM (1 mL) was added DMAP (7 mg), and the mixture was stirred at room temperature for 30 min. The mixture was cooled to 0° C., followed by addition of a solution of H₂NSO₂Me (2.6 mg) and compound 10 (10.0 mg) in DCM (1 mL), and the mixture was stirred at room temperature overnight. Then the mixture was quenched with saturated NaHCO₃ solution, extracted with EA, and the combined organic layers were dried over anhydrous Na₂SO₄, filtered, and concentrated in vacuo. The crude product was purified by silica gel chromatography to give the desired product 4-2 (7.0 mg, 57%). ¹H NMR (400 MHz, Chloroform-d) δ 3.69 (s, 1H), 3.47 (q, J=7.4 Hz, 2H), 2.42-2.33 (m, 1H), 2.28-2.19 (m, 1H), 0.95-0.87 (m, 9H), 0.66 (s, 3H).

### Example 19. Synthesis of Compound 4-3

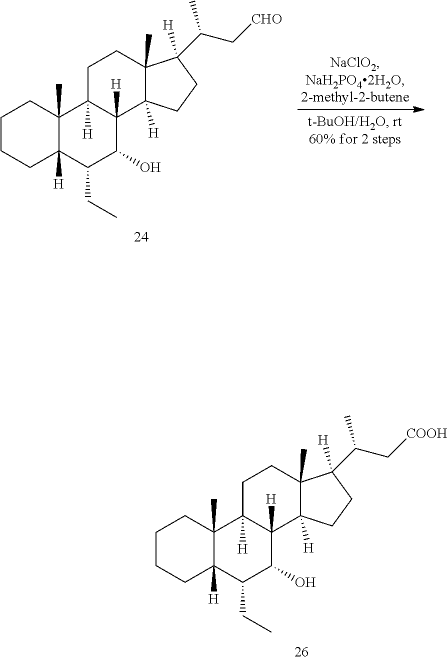

Step 1: Synthesis of compound 26. To a solution of 24 (60 mg) in t-BuOH/H₂O (4:1, 1.5 mL) was added 2-methyl-2-butene (113 μL), NaH₂PO₄·2H₂O (168 mg) and NaClO₂ (61 mg), and the mixture was stirred at room temperature for 2 h. The mixture was quenched with 1M HCl solution, and extracted with EA. The combined organic layers were dried over anhydrous Na₂SO₄, filtered, and concentrated in vacuo. The crude product was purified by silica gel chromatography to give the desired product 26 (30 mg, 60% for 2 steps).

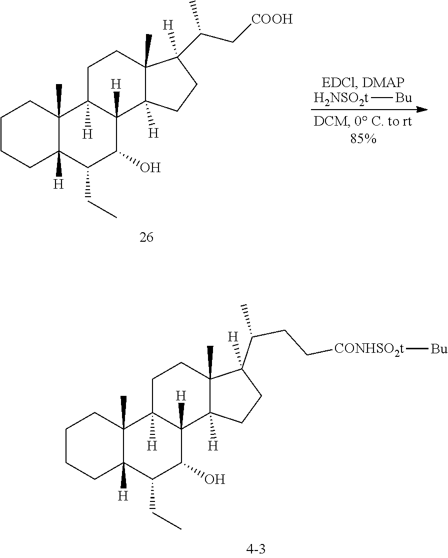

Step 2: Synthesis of 4-3. To a solution of EDCI (7.7 mg) in DCM (1 mL) was added DMAP (9.0 mg), and the mixture was stirred at room temperature for 30 min. The mixture was cooled to 0° C., followed by addition of a solution of H₂NSO₂t-Bu (4.2 mg) and compound 26 (12.0 mg) in DCM (1 mL), and the mixture was stirred at room temperature overnight. Then the mixture was quenched with saturated NaHCO₃ solution, extracted with EA, and the combined organic layers were dried over anhydrous Na₂SO₄, filtered, and concentrated in vacuo. The crude product was purified by silica gel chromatography to give the desired product 4-3 (13.3 mg, 85%). ¹H NMR (400 MHz, Chloroform-d) δ 3.69 (s, 1H), 2.52 (d, J=11.6 Hz, 1H), 2.11-1.50 (m, 17H), 1.48 (s, 9H), 1.46-0.86 (m, 20H), 0.70 (s, 3H).

A general scheme for synthesis of compounds 5-x series as an example:

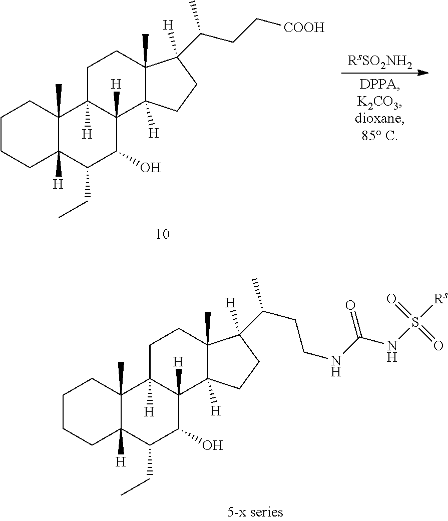

R^{s}(O)₂NH₂ (as reagents; also R^{s}S(O)₂NH— in products):

A general synthetic procedure for the 5-x series:

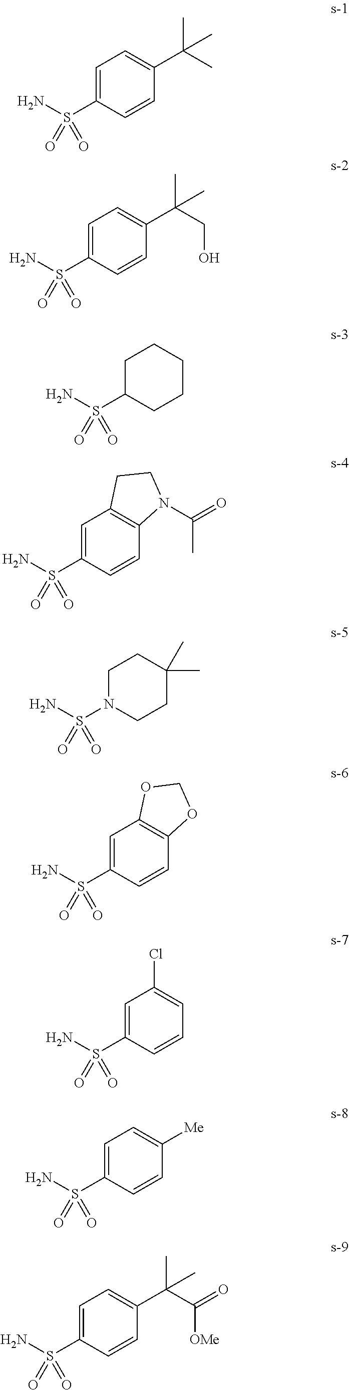

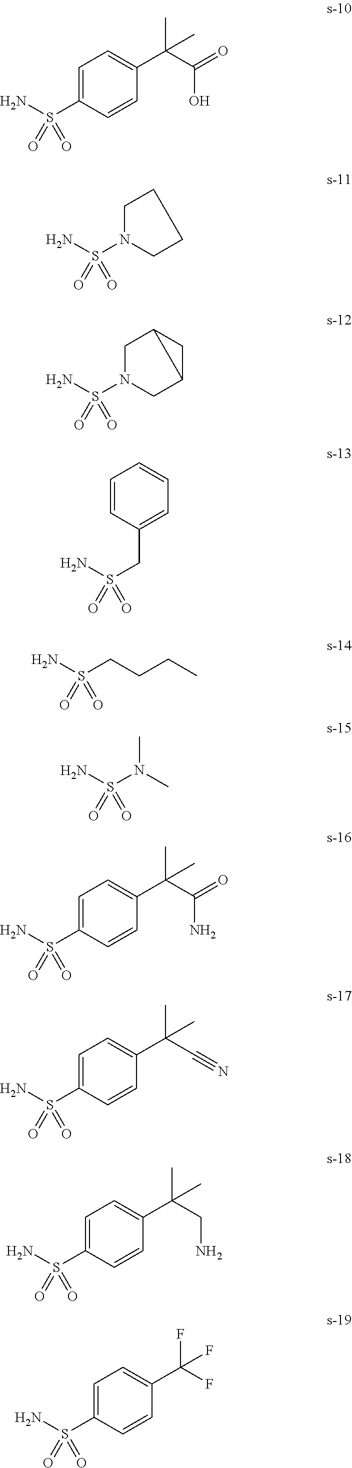

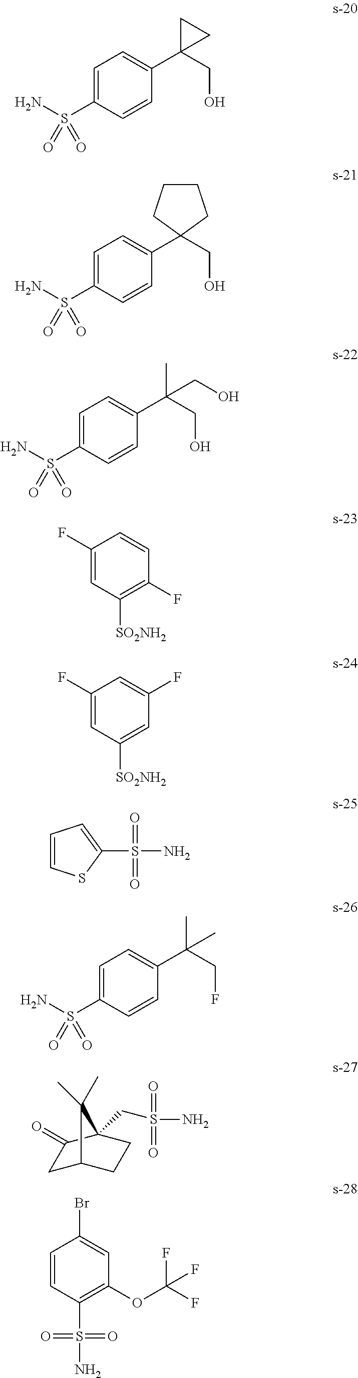

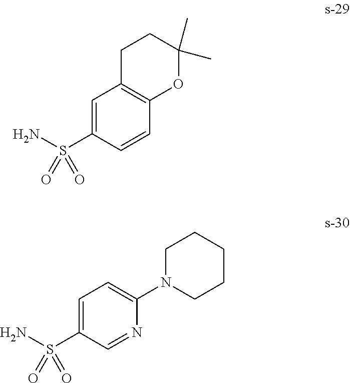 [SMILES: CC1(C)CCc2cc(S(N)(=O)=O)ccc2O1 ; NS(=O)(=O)c1ccc(N2CCCCC2)nc1]

To a solution of compound 10 in dioxane was added sulfonamide intermediates (1 eq), K₂CO₃ (3 eq) and DPPA (1.2 eq) in return under Ar₂ at rt, then heated to 85° C. for 3 h. The mixture was washed with H₂O, brine, dried over anhydrous Na₂SO₄, and concentrated in vacuo. The residue was purified by silica gel column chromatography to obtain target compounds 5-x series.

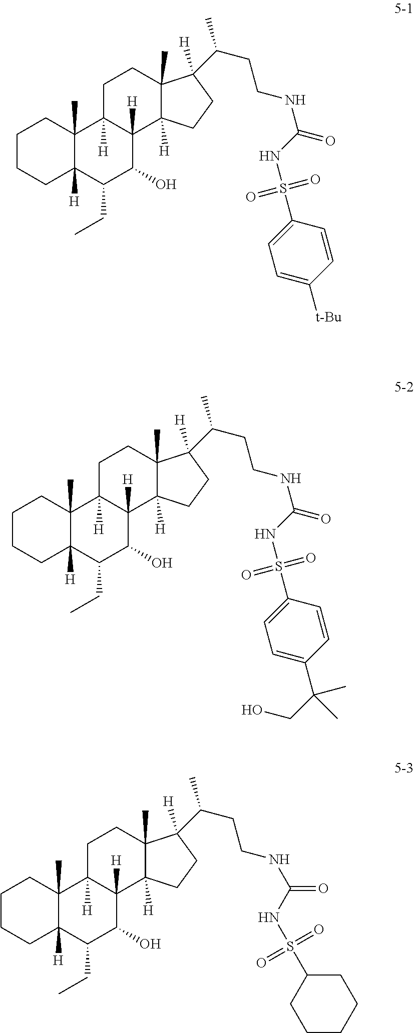

Example 20, compound 5-1: 1H NMR (400 MHz, CDCl3) δ 7.65 (d, J=8.6 Hz, 2H), 7.56 (d, J=8.6 Hz, 2H), 3.74 (s, J=9.1, 3.74 Hz, 4H), 3.34 (m, 1H), 3.14 (d, J=21.0 Hz, 2H), 0.92-2.00 (m, 29H), 0.84-0.88 (m, 9H).

Example 21, compound 5-2: ¹H NMR (400 MHz, CDCl₃) δ 7.93-7.73 (m, 2H), 7.60-7.40 (m, 2H), 6.47-6.30 (m, 1H), 3.67 (d, J=2.8 Hz, 1H), 3.61 (s, 2H), 3.27-3.20 (m, 1H), 3.14-3.03 (m, 1H), 1.96-1.30 (m, 23H), 1.27-0.87 (m, 19H), 0.63 (s, 5H).

Example 22, compound 5-3: ¹H NMR (400 MHz, Chloroform-d) δ 6.49 (t, J=5.5 Hz, 1H), 3.68 (t, J=2.6 Hz, 1H), 3.35-3.27 (m, 1H), 3.23-3.07 (m, 2H), 2.25-2.11 (m, 2H), 1.98-1.84 (m, 4H), 1.83-0.86 (m, 40H), 0.65 (s, 3H).

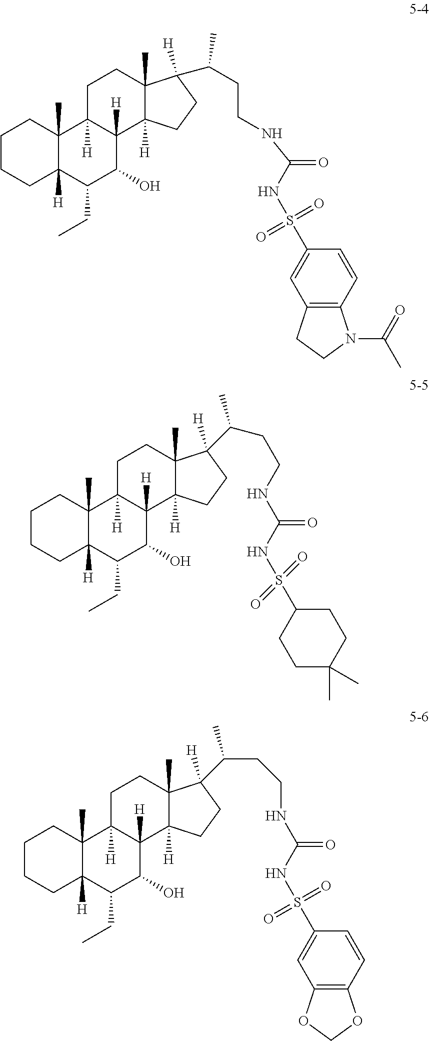

Example 23, compound 5-4: ¹H NMR (400 MHz, Chloroform-d) δ 8.26 (d, J=8.5 Hz, 1H), 7.69 (dd, J=11.5, 3.3 Hz, 2H), 6.43 (s, 1H), 4.14 (t, J=8.6 Hz, 2H), 3.68 (d, J=2.9 Hz, 1H), 3.34-3.17 (m, 3H), 3.14-3.08 (m, 1H), 2.26 (s, 3H), 1.97-1.31 (m, 20H), 1.23-0.86 (m, 16H), 0.63 (s, 3H).

Example 24, compound 5-5: ¹H NMR (400 MHz, Chloroform-d) δ 6.42 (t, J=5.6 Hz, 1H), 3.69 (d, J=2.9 Hz, 1H), 3.37-3.11 (m, 1H), 1.97-1.59 (m, 9H), 1.52-0.86 (m, 29H), 0.65 (s, 3H).

Example 25, compound 5-6: ¹H NMR (400 MHz, Chloroform-d) δ 7.47 (ddd, J=10.2, 8.2, 2.0 Hz, 1H), 7.30 (dd, J=14.7, 1.9 Hz, 1H), 6.86 (d, J=8.2 Hz, 1H), 6.46 (t, J=5.4 Hz, 1H), 6.08 (d, J=7.2 Hz, 2H), 3.69 (d, J=2.8 Hz, 1H), 3.29 (ddt, J=14.4, 10.0, 5.1 Hz, 1H), 3.22-3.10 (m, 1H), 1.96-1.57 (m, 5H), 1.51-0.86 (m, 19H), 0.64 (s, 3H).

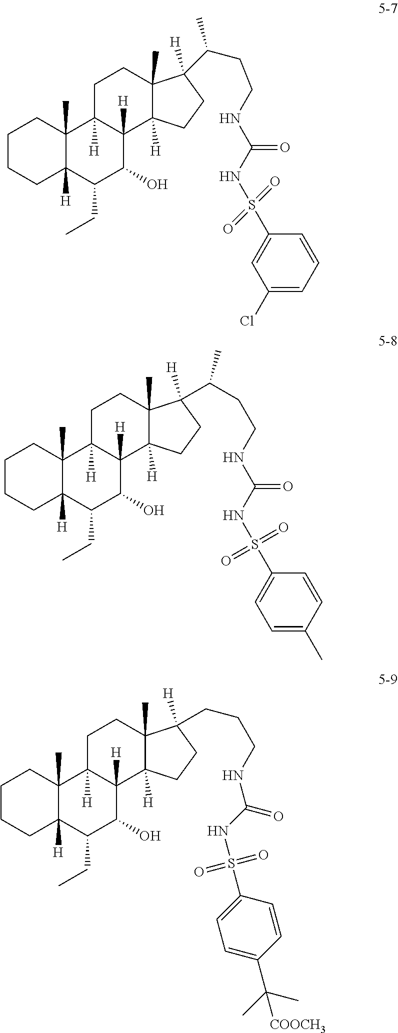

Example 26, compound 5-7: ¹H NMR (400 MHz, Chloroform-d) δ 7.88-7.86 (s, 1H), 7.80-7.74 (m, 1H), 7.62-7.59 (m, 1H), 7.48 (t, J=8.0 Hz, 1H), 6.48 (s, 1H), 3.71-3.69 (m, 1H), 3.35-3.27 (m, 1H), 3.20-3.15 (m, 10H), 1.97-1.58 (m, 20H), 1.53-1.10 (m, 6H), 0.65 (s, 3H).

Example 27, compound 5-8: ¹H NMR (400 MHz, Chloroform-d) (7.76 (d, J=8.4 Hz, 2H), 7.31 (d, J=8.0 Hz, 2H), 6.48-6.30 (m, 1H), 3.69 (m, 1H), 3.32-3.24 (m, 1H), 3.20-3.10 (m, 1H), 2.44 (s, 2H), 1.98-1.55 (m, 11H), 1.53-0.90 (m, 25H), 0.64 (s, 3H).

Example 28, compound 5-9: ¹H NMR (400 MHz, Chloroform-d) (57.86 (dd, J=12.6, 8.7 Hz, 1H), 7.52-7.41 (m, 1H), 6.51 (t, J=5.4 Hz, 1H), 3.69 (t, J=2.4 Hz, 1H), 3.67 (d, J=2.0 Hz, 3H), 3.33-3.25 (m, 1H), 3.20-3.09 (m, 1H), 1.97-1.63 (m, 8H), 1.60 (s, 6H), 1.53-0.89 (m, 28H), 0.65 (s, 3H).

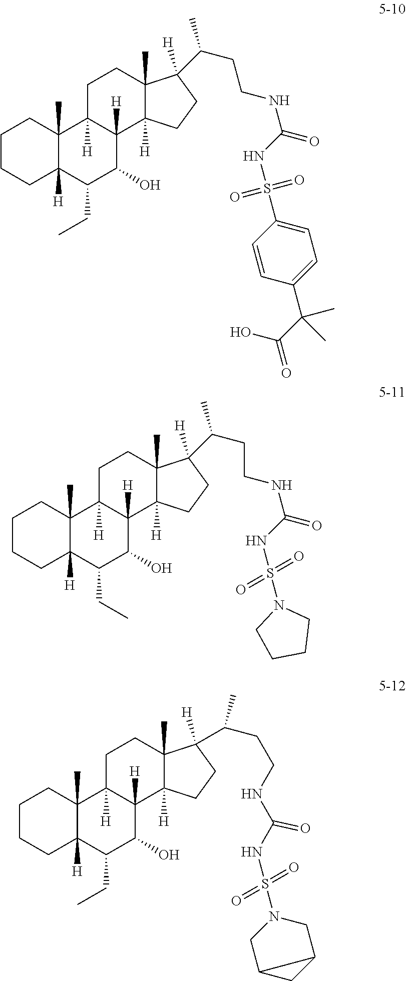

### Example 29: Synthesis of Compound 5-10

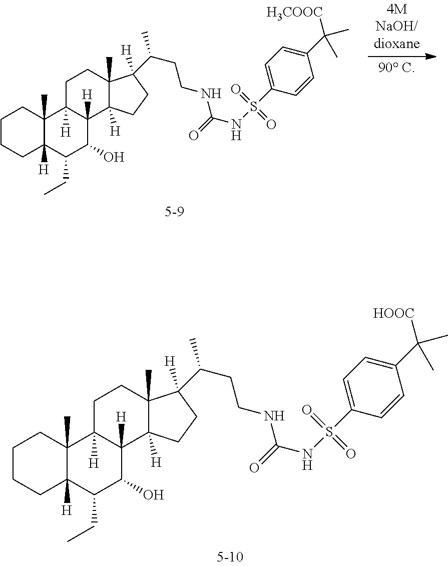

To a solution of 5-9 (47 mg) in dioxane (2 mL) was added 4 M NaOH (4 mL) and heated at 90° C. rt overnight. The reaction mixture was diluted with 4 M HCl and brine. The organic layer was dried and concentrated under vacuum. The crude product was purified to obtain 5-10 (26 mg). ¹H NMR (400 MHz, CDCl₃) δ 7.83 (d, J=8.6 Hz, 2H), 7.56 (d, J=8.6 Hz, 2H), 6.82 (t, J=5.5 Hz, 1H), 3.70 (s, 1H), 3.25 (dd, J=9.1, 4.7 Hz, 1H), 3.18-3.07 (m, 1H), 2.22 (d, J=21.0 Hz, 2H), 1.97-1.66 (m, 6H), 1.51-1.12 (m, 34H), 0.65 (s, 2H).

Example 30, compound 5-11: ¹H NMR (400 MHz, Chloroform-d) δ 6.43 (s, 1H), 3.68 (s, 1H), 3.41-3.26 (m, 5H), 3.23-3.12 (m, 1H), 1.98-1.90 (m, 4H), 1.81-1.59 (m, 9H), 1.53-0.88 (m, 27H), 0.65 (s, 3H).

Example 31, compound 5-12: ¹H NMR (400 MHz, Chloroform-d) δ 6.29 (m, 1H), 3.65 (d, J=3.0 Hz, 1H), 3.47-3.37 (m, 4H), 3.29-3.22 (m, 1H), 3.12-3.08 (m, 1H), 1.95-1.55 (m, 9H), 1.54-1.13 (m, 20H), 1.10-0.86 (m, 11H), 0.63 (s, 3H).

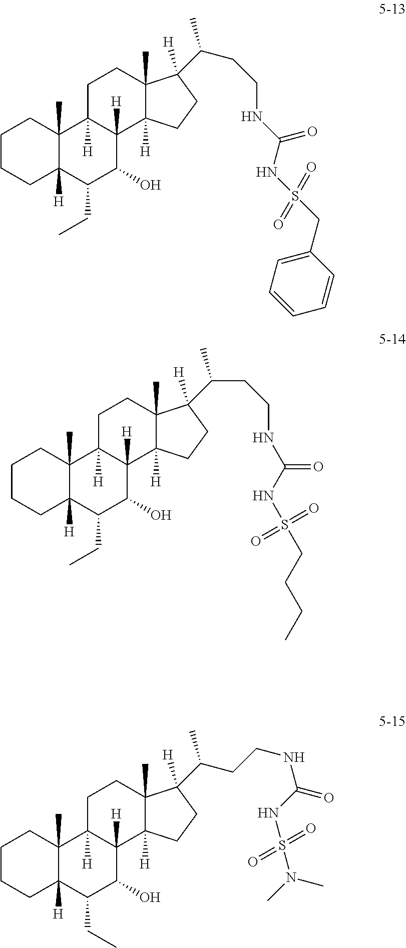

Example 32, compound 5-13: ¹H NMR (400 MHz, Chloroform-d) δ 7.37 (s, 5H), 7.36 (d, J=1.4 Hz, 1H), 5.99 (t, J=5.4 Hz, 1H), 4.45 (s, 2H), 3.68 (t, J=2.7 Hz, 1H), 3.12-3.02 (m, 1H), 2.97-2.87 (m, 1H), 2.00-1.59 (m, 10H), 1.55-0.88 (m, 26H), 0.64 (s, 3H).

Example 33, compound 5-14: ¹H NMR (400 MHz, Chloroform-d) δ 6.40-6.28 (m, 1H), 3.68 (s, 1H), 3.38-3.05 (m, 4H), 2.00-1.58 (m, 10H), 1.54-0.82 (m, 30H), 0.64 (s, 3H).

Example 34, compound 5-15: ¹H NMR (400 MHz, Chloroform-d) δ 6.47-6.30 (m, 1H), 3.68 (d, J=2.9 Hz, 1H), 3.35-3.27 (m, 1H), 3.26-3.16 (m, 1H), 2.88 (s, 6H), 1.97-1.63 (m, 10H), 1.52-1.15 (m, 20H), 0.99-0.87 (m, 6H), 0.65 (s, 3H).

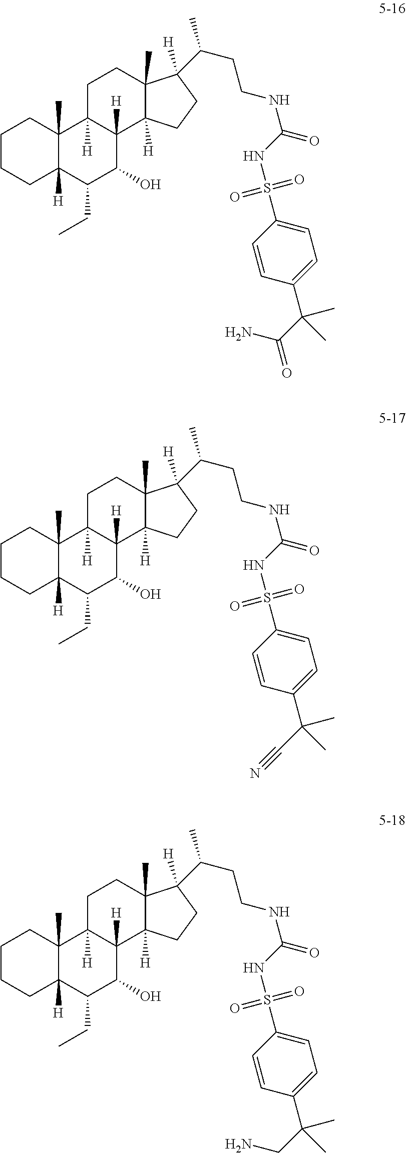

Example 35, compound 5-16: ¹H NMR (400 MHz, Chloroform-d) δ 7.73 (d, J=8.6 Hz, 2H), 7.69 (d, J=8.6 Hz, 2H), 7.21 (t, J=5.5 Hz, 1H), 3.74 (s, J=9.1, 3.74 Hz, 4H), 3.70 (s, 1H), 3.34 (m, 1H), 3.14 (d, J=21.0 Hz, 2H), 0.92-2.00 (m, 38H), 0.84-0.88 (m, 9H).

Example 36, compound 5-17: ¹H NMR (400 MHz, Chloroform-d) δ 7.93 (d, J=8.7 Hz, 2H), 7.67 (s, 2H), 6.50 (t, J=5.0 Hz, 1H), 3.70 (d, J=2.9 Hz, 1H), 3.30 (dt, J=9.2, 4.3 Hz, 1H), 3.22-3.13 (m, 1H), 1.97-1.79 (m, 4H) 1.73-1.58 (m, 6H), 1.54-1.33 (m, 10H), 1.29-1.12 (m, 12H), 1.06-0.91 (m, 10H), 0.65 (s, 2H).

Example 37, compound 5-18: LC-MS, ES⁺ (m/z): 630.93 (M+1).

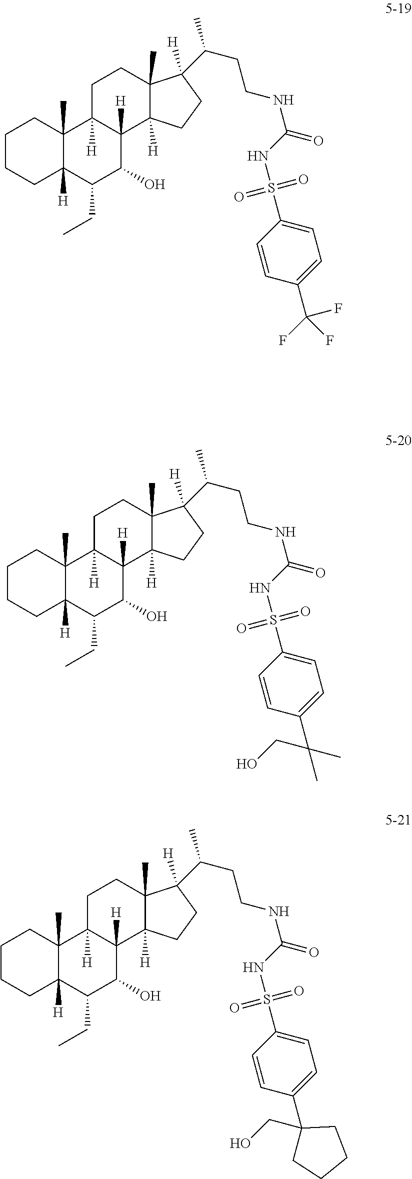

Example 38, compound 5-19: LC-MS, ES⁺ (m/z): 627.78 (M+1).

Example 39, compound 5-20: ¹H NMR (400 MHz, CDCl3) δ 7.83 (d, J=8.6 Hz, 2H), 7.56 (d, J=8.6 Hz, 2H), 6.82 (t, J=5.5 Hz, 1H), 3.70 (s, 1H), 3.25 (dd, J=9.1, 4.7 Hz, 1H), 3.18-3.07 (m, 1H), 2.22 (d, J=21.0 Hz, 2H), 1.97-1.66 (m, 6H), 1.51-1.12 (m, 34H), 0.65 (s, 2H).

Example 40, compound 5-21: ¹H NMR (400 MHz, CDCl3) δ 7.65 (d, J=8.6 Hz, 2H), 7.56 (d, J=8.6 Hz, 2H), 3.70 (s, 1H), 3.74 (s, J=9.1, 3.74 Hz, 2H), 3.34 (m, 1H), 3.14 (d, J=21.0 Hz, 2H), 0.92-2.00 (m, 38H), 0.84-0.88 (m, 9H).

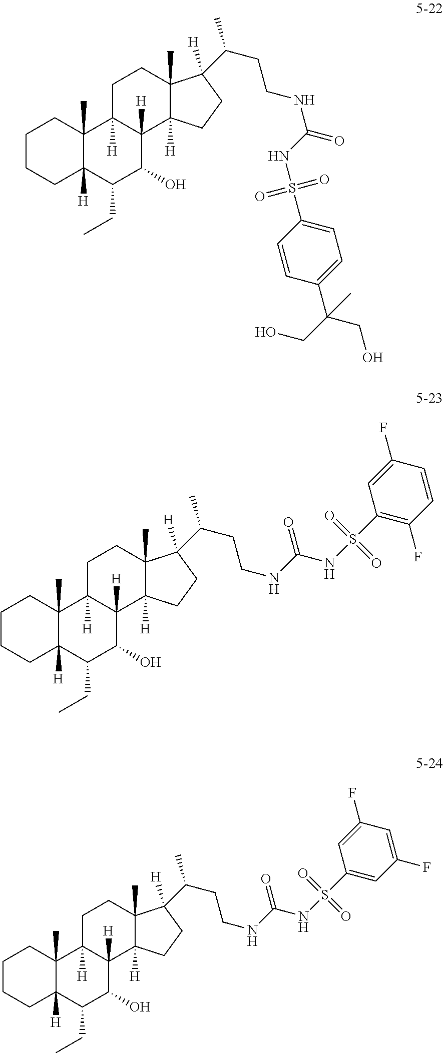

Example 41, compound 5-22: H NMR (400 MHz, CDCl3) δ 7.65 (d, J=8.6 Hz, 2H), 7.56 (d, J=8.6 Hz, 2H), 3.74 (s, J=9.1, 3.74 Hz, 4H), 3.70 (s, 1H), 3.34 (m, 1H), 3.14 (d, J=21.0 Hz, 2H), 0.92-2.00 (m, 33H), 0.84-0.88 (m, 9H).

Example 42, compound 5-23: LC-MS, ES⁻ (m/z): 593.35 (M-1). ¹H NMR (400 MHz, Chloroform-d) δ 7.62 (m, 1H), 7.38-7.29 (m, 1H), 7.22 (td, J=9.0, 4.1 Hz, 1H), 6.47-6.37 (m, 1H), 3.69 (t, J=2.7 Hz, 1H), 3.29 (m, 1H), 3.16 (m, 1H), 1.99-1.08 (m, 27H), 0.95-0.85 (m, 9H), 0.64 (s, 3H).

Example 43, compound 5-24: LC-MS, ES⁻ (m/z): 593.33 (M-1). ¹H NMR (400 MHz, Chloroform-d) δ 7.53-7.49 (m, 1H), 7.42 (m, 2H), 7.34 (t, J=2.2 Hz, 1H), 7.14-7.07 (m, 1H), 3.68 (d, J=3.0 Hz, 1H), 3.38-3.11 (m, 2H), 1.96-1.08 (m, 27H), 0.94 (d, J=6.6 Hz, 3H), 0.91-0.86 (m, 6H), 0.64 (s, 3H).

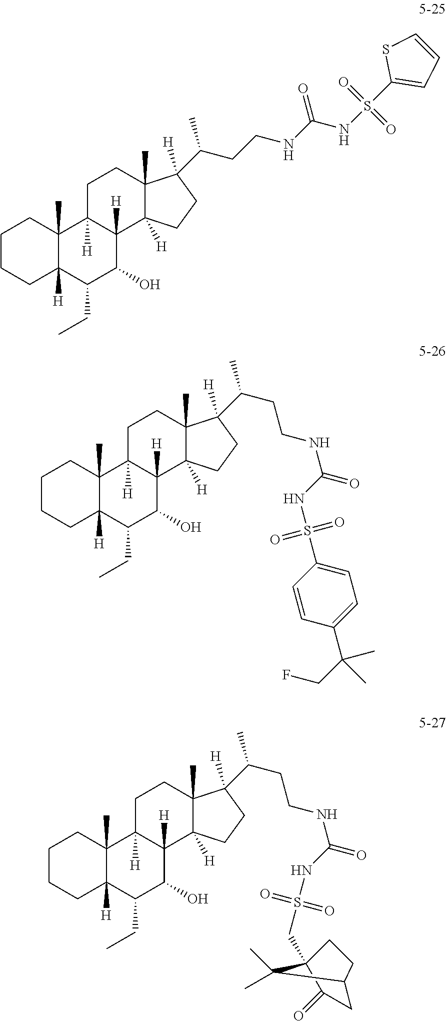

Example 44, compound 5-25: LC-MS, ES⁺ (m/z): 587.35 (M+23). ¹H NMR (400 MHz, Chloroform-d) δ 7.69 (dd, J=3.8, 1.4 Hz, 1H), 7.64 (dd, J=5.0, 1.4 Hz, 1H), 7.10 (dd, J=5.0, 3.8 Hz, 1H), 6.50 (t, J=5.5 Hz, 1H), 3.69 (t, J=2.8 Hz, 1H), 3.39-3.12 (m, 2H), 1.99-1.07 (m, 27H), 0.95 (d, J=6.6 Hz, 3H), 0.92-0.87 (m, 6H), 0.65 (s, 3H).

Example 45, compound 5-26: LC-MS, ES⁻ (m/z): 625.78 (M-1). ¹H NMR (400 MHz, Chloroform-d) δ 7.84 (dd, J=13.8, 8.4 Hz, 2H), 7.45-7.31 (m, 2H), 6.51 (s, 1H), 3.69 (d, J=2.8 Hz, 1H), 3.29 (s, 1H), 3.21-3.10 (m, 1H), 3.00 (d, J=2.4 Hz, 1H), 2.94 (d, J=2.3 Hz, 1H), 1.98-1.57 (m, 9H), 1.53-0.90 (m, 33H), 0.65 (s, 3H).

Example 46, compound 5-27: LC-MS, ES⁺ (m/z): 633.93 (M+1).

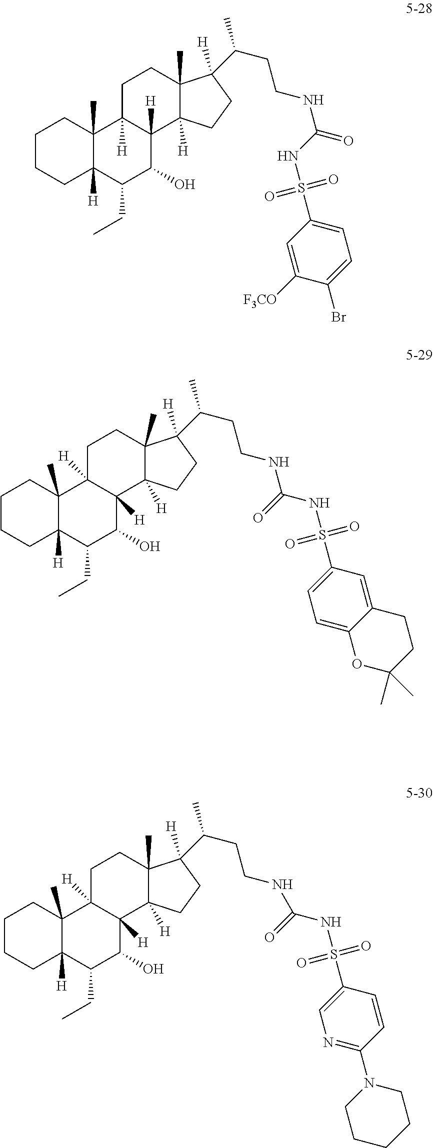

Example 47, compound 5-28: LC-MS, ES⁺ (m/z): 721.24, 723.24 (M+1).

Example 48, compound 5-29: 1H NMR (400 MHz, Chloroform-d) δ 7.78 (d, J=8.4 Hz, 2H), 7.49 (m, 1H), 6.55 (s, 1H), 3.71-3.64 (m, 1H), 3.38-3.09 (m, 2H), 2.96 (d, J=21.6 Hz, 2H), 2.00-1.06 (m, 35H), 0.94 (d, J=6.6 Hz, 3H), 0.90-0.85 (m, 6H), 0.64 (s, 3H).

Example 49, compound 5-30: 1H NMR (400 MHz, Chloroform-d) δ 7.83 (m, 1H), 7.29 (m, 1H), 7.20 (td, J=9.0, 4.1 Hz, 1H), 6.43-6.34 (m, 1H), 3.70 (t, J=2.7 Hz, 1H), 3.31 (m, 1H), 3.18 (m, 1H), 2.02-1.09 (m, 37H), 0.96-0.87 (m, 9H), 0.66 (s, 3H).

Example 50. Synthesis of sulfonamide intermediates s-9 and s-2.

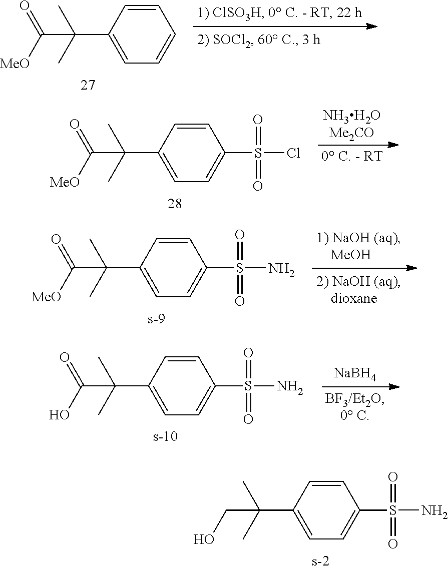 [SMILES: COC(=O)C(C)(C)c1ccccc1 ; COC(=O)C(C)(C)c1ccc(S(=O)(=O)Cl)cc1 ; COC(=O)C(C)(C)c1ccc(S(N)(=O)=O)cc1 ; CC(C)(C(=O)O)c1ccc(S(N)(=O)=O)cc1 ; "NaBH4" ; CC(C)(CO)c1ccc(S(N)(=O)=O)cc1]

Step 1: Synthesis of compound 28. To a solution of ethyl 2-methyl-2-phenylpropionate 27 (1 g, 5.2 mmol) in DCM (10 mL) was added ClSO₃H (0.93 mL, 14.0 mmol) dropwise over 3 min at 0° C., and the mixture was allowed to stir at room temperature for 22 h. After evaporation of the volatile, SOCl₂ (10.8 mL) was added, and the reaction mixture was stirred at 60° C. for 3 h. The volatile was evaporated. The residue was poured into ice-water and extracted with ether. The organic extract was washed with H₂O and brine, dried over anhydrous MgSO4, and concentrated in vacuo to afford crude 28 (1.43 g).
Step 2: Synthesis of s-9. To a solution of the obtained 28 (1.43 g) in Me₂CO (6 mL) was added concentrated NH₄OH (9 mL). The mixture was stirred at room temperature for 2 h and acidified with 10% aqueous HCl. The organic layer was separated, washed with H₂O and brine, dried over anhydrous Na₂SO₄, and concentrated in vacuo, and concentrated in vacuo to afford s-9 (0.72 g, 49%).
Step 3: Synthesis of compound s-10. To a solution of s-9 (500 mg, 1.95 mmol) in dioxane (4 mL) was added 1M NaOH (8 mL) at 0° C. The mixture was allowed to stir at 95° C. overnight, acidified with 10% aqueous HCl, saturated with NaCl, and extracted twice with EtOAc. The combined organic extracts were washed with brine, dried over anhydrous Na₂SO₄, and concentrated in vacuo. The residue was purified by silica gel column chromatography to obtain compound s-10 (0.39 g, 83%).
Step 4: Synthesis of s-2. To a suspension of NaBH₄ (0.183 g, 4.81 mmol) in dry THF (5 mL) was added BF₃/Et₂O (0.65 mL, 5.29 mmol) dropwise at 0° C., and the mixture was stirred at the same temperature for 30 min. A solution of compound s-10 (0.39 g, 1.6 mmol) in dry THF (5 mL) was added dropwise at the same temperature over 30 min, and the mixture was stirred at room temperature for 3 h. MeOH was added to the reaction mixture until H₂ gas generation stopped. The mixture was diluted with 10% aqueous HCl and extracted twice with EtOAc. The combined organic extracts were washed with H₂O and brine, dried over anhydrous Na₂SO₄, and concentrated in vacuo. The residue was purified by silica gel column chromatography to afford s-2 (0.32 g, 87%).

### Example 51. Synthesis of Sulfonamide Intermediates s-4

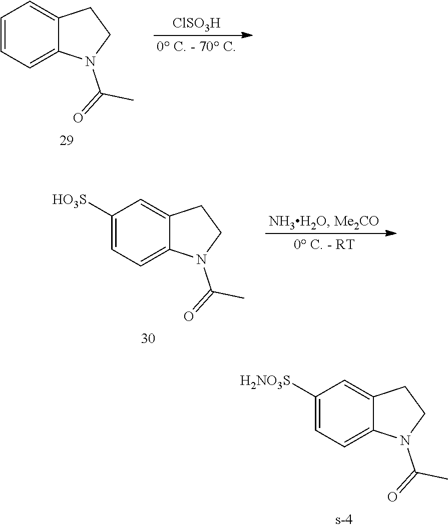

Step 1: Synthesis of 29. N-acetyl indoline 29 (0.50 g, 3.11 mmol) at 0° C. was added ClSO₃H (1.5 mL, 0.043 mmol) dropwise. The reaction mixture was warmed to 70° C. for 2 h. Then the reaction mixture was cooled to room temperature and carefully poured into 50 ml ice. The solid was performed, filtered, washed with cool water and dried to afford 30 (0.785 g, 92%).
Step 2: Synthesis of s-4. To a solution of 30 (0.785 g) in acetone (6 mL) at 0° C. was added ammonium hydroxide (36-38%) (9 mL), and then warmed to rt for 3 h. The solid was performed, filtered, washed with cool water and dried to afford s-4 (0.62 g, 87%)

### Example 52. Synthesis of Sulfonamide Intermediates s-14

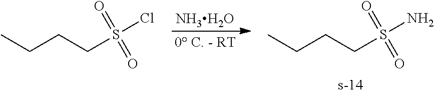

To a solution of n-butane-sulfonyl chloride (0.5 g) in acetone (6 mL) at 0° C. was added ammonium hydroxide (36-38%) (9 mL), and then warmed to rt for 3 h. The solid was performed, filtered, washed with cool water and dried to afford s-14 (0.41 g, 93%).

### Example 53. Synthesis of Sulfonamide Intermediates s-11

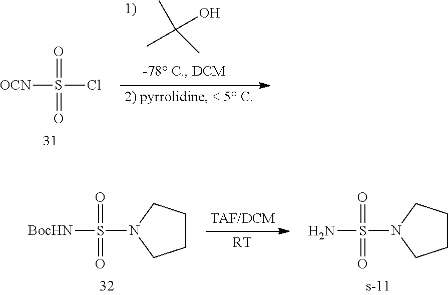

Step 1: Synthesis of 32. To a solution of chloro-sulfonyl isocyanate 31 (1.0 g, 7.1 mol) m anhydrous DCM (20 mL) at −78° C. was added absolute tert-butanol (0.71 mL, 7.1 mmol) slowly and maintained for 30 min, then the solution was allowed to reach 5° C. and stirred for 2 h. The resulting solution was added of triethylamine (2.97 mL, 21.3 mmol) in 20 mL DCM were added dropwise to pyrrolidine (0.583 mL, 7.1 mmol) in 20 mL of DCM, maintaining the reaction temperature below 5° C. The resulting reaction solution was allowed to warm up to rt over 3 h. The reaction mixture was diluted with 100 mL of dichloromethane, washed with 0.1 N HCl solution and brine. The organic layer was dried in Na₂SO₄, filtered and concentrated under vacuum. The crude product was purified by silica gel chromatography to obtain 32 (0.55 g, 67%).
Step 2: Synthesis of s-11. To a solution of 32 (0.55 g) in DCM (10 mL) was added TFA (20 mL) slowly and stirred at rt for 2 h, The reaction mixture was concentrated in vacuo to afford s-11 (0.29 g, 88%).

### Example 54. Synthesis of Sulfonamide Intermediates s-6

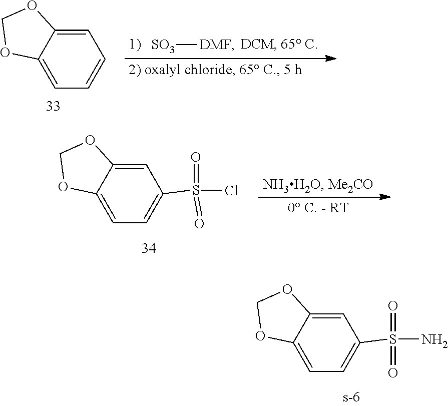

Step 1: Synthesis of 34. To a suspension of SO₃-DMF complex (2.78 g, 18.5 mmol) in DCE (16 mL) was added a solution of 1,3-benzodioxole 33 (2.0 g, 16.4 mmol) in DCE (16 mL) dropwise and heated to 75° C. After 18 h, the reaction mixture was cooled to room temperature and treated dropwise with oxalyl chloride (1.54 mL, 18.5 mmol) and heated to 65° C. After 2 h, the reaction mixture was quenched by the cool water to form precipitation and filtrated to afford 34 (3.19 g, 89%).
Step 2: Synthesis of s-6. To a solution of 34 (3.19 g) in acetone (10 mL) at 0° C. was added ammonium hydroxide (36-38%) (15 mL), and then warmed to rt for 3 h. The solid was performed, filtered, washed with cool water and dried to afford s-6 (2.76 g, 94%).

### Example 55. Synthesis of Sulfonamide Intermediates s-5

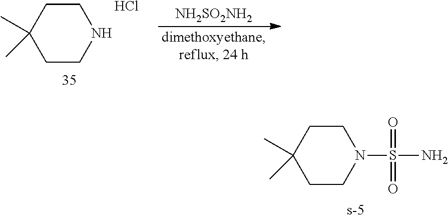

To a solution of 4,4-dimethylpiperidine hydrochloride 35 (100 mg, 0.88 mmol) and TEA (123 L, 0.88 mmol) in dimethoxy-ethane (3 mL) was added sulfonamide (85 mg, 0.88 mmol), and heated under reflux on a steam bath overnight. Then purified by silica gel column chromatography to afford s-5 (55 mg, 33%).

### Example 56. Synthesis of Sulfonamide Intermediates s-8

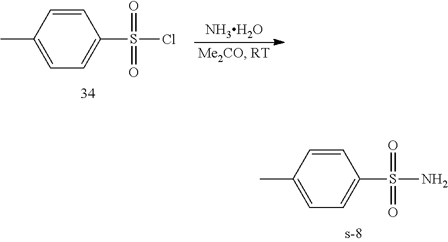

To a solution of p-toluene-sulfonyl chloride 36 (100 mg) in acetone (2 mL) at 0° C. was added ammonium hydroxide (36-38%) (3 mL), and then warmed to rt for 3 h. The solid was performed, filtered, washed with cool water and dried to afford s-8 (65 mg, 72%).

### Example 57. Synthesis of Sulfonamide Intermediates s-12

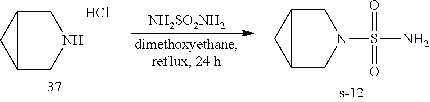 [SMILES: C1NCC2CC12 ; Cl ; "NH2SO2NH2" ; NS(=O)(=O)N1CC2CC2C1]

To a solution of 3-azabicyclo-[3.1.0]-hexane hydrochloride 37 (100 mg, 0.84 mmol) and TEA (117 μL, 0.84 mmol) in dimethoxy-ethane (3 mL) was added sulfonamide (81 mg, 0.84 mmol), and heated under reflux on a steam bath overnight. Then purified by silica gel column chromatography to afford s-12 (55 mg, 40%).

### Example 58. Synthesis of Sulfonamide Intermediates s-16

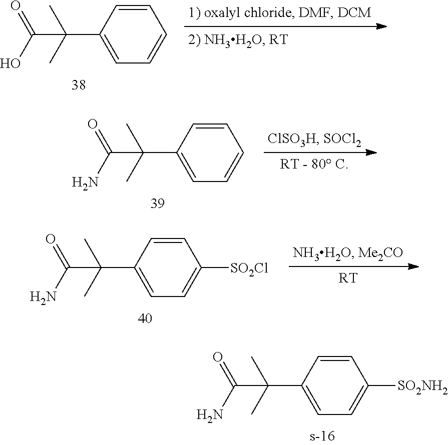 [SMILES: CC(C)(C(=O)O)c1ccccc1 ; CC(C)(C(N)=O)c1ccccc1 ; CC(C)(C(N)=O)c1ccc(S(=O)(=O)Cl)cc1 ; "RT" ; CC(C)(C(N)=O)c1ccc(S(N)(=O)=O)cc1]

Step 1: Synthesis of 39. To a solution of 2-methyl-2-phenylpropanoic acid 38 (1 g, 6.1 mmol) in DCM (20 mL) was added 2 drops DMF and oxalyl chloride (6 mL) at 0° C., and the mixture was allowed to stir at room temperature overnight. Then NH₃H₂O (10.8 mL) was added and stirred drastically. The volatile was evaporated in vacuo to afford crude 39 (0.94 g).
Step 2: Synthesis of 40. To a solution of 39 (1 g, 5.2 mmol) in ClSO₃H (6 mL) was added SOCl₂ (2 mL) dropwise at 0° C. and heated at 80° C. for 2 h. The volatile was evaporated. The residue was poured into ice-water and extracted with ether. The organic extract was washed with H₂O and brine, dried over anhydrous MgSO4, and concentrated in vacuo to afford crude 40.
Step 3: Synthesis of s-16. To a solution of the obtained 40 (100 mg) in Me₂CO (3 mL) was added concentrated NH₄OH (1 mL). The mixture was stirred at room temperature for 2 h and acidified with 10% aqueous HCl. The organic layer was separated, washed with H₂O and brine, dried over anhydrous Na₂SO₄, and concentrated in vacuo, and concentrated in vacuo to afford s-16 (45 mg).

### Example 59. Synthesis of Sulfonamide Intermediates s-26

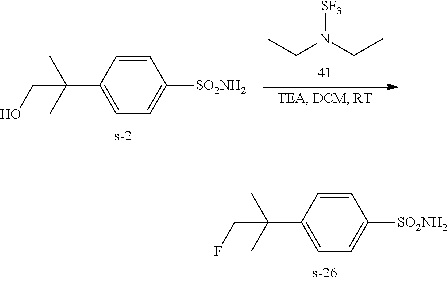

To a solution of 4-(1-hydroxy-2-methylpropan-2-yl)-benzene-sulfonamide s-2 (100 mg) in DCM (3 mL) at −15° C. was added TEA (2.0 eq.) and N, N-diethyl-1,1,1-trifluoro-14-sulfanamine 41 (1.5 eq.) in turn, then warmed to rt for 3 h. The crude product was washed with cool water and purified with silica gel chromatography to obtain s-26 (42 mg).

### Example 60. Synthesis of Sulfonamide Intermediates s-17

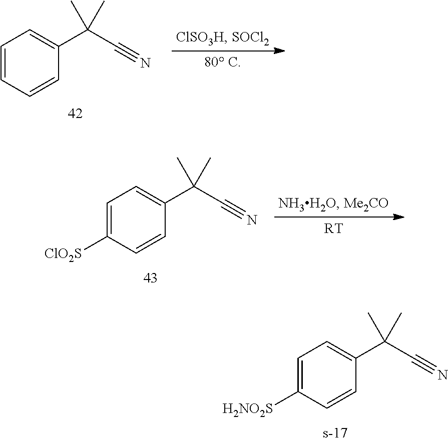

Step 1: Synthesis of 43. To a solution of 2-methyl-2-phenylpropanenitrile 42 (100 mg) in ClSO₃H (1.5 mL) was added SOCl₂ (0.5 mL) dropwise at 0° C. and heated at 80° C. for 2 h. The volatile was evaporated. The residue was poured into ice-water and extracted with ether. The organic extract was washed with H₂O and brine, dried over anhydrous MgSO4, and concentrated in vacuo to afford crude 43.
Step 2: Synthesis of s-17. To a solution of 43 in acetone (3 mL) at 0° C. was added ammonium hydroxide (36-38%) (1 mL), and then warmed to rt for 3 h. The solid was performed, filtered, washed with cool water and dried to afford s-17 (96 mg).

### Example 61. Synthesis of Sulfonamide Intermediate s-21

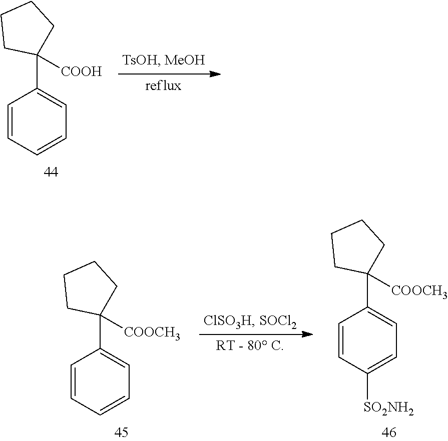 [SMILES: O=C(O)C1(c2ccccc2)CCCC1 ; COC(=O)C1(c2ccccc2)CCCC1 ; COC(=O)C1(c2ccc(S(N)(=O)=O)cc2)CCCC1]

Step 1: Synthesis of 45. To a solution of 1-phenylcyclopentane-1-carboxylic acid 44 (1 g) in anhydrous MeOH (10 mL) at RT was added TsOH (2.0 eq.) and reflux overnight. The reaction mixture was diluted with sat. NaHCO₃ and brine. The organic layer was dried and concentrated under vacuum. The crude product was purified with silica gel chromatography to obtain 45 (0.89 g).
Step 2: Synthesis of 46. To a solution of 45 (0.89 g) in ClSO₃H (6 mL) was added SOCl₂ (2 mL) dropwise at 0° C. and heated at 80° C. for 2 h. The volatile was evaporated and the residue was poured into ice-water and extracted with ether. The organic extract was washed with H₂O and brine, dried over anhydrous MgSO4, and concentrated in vacuo to afford crude 46.

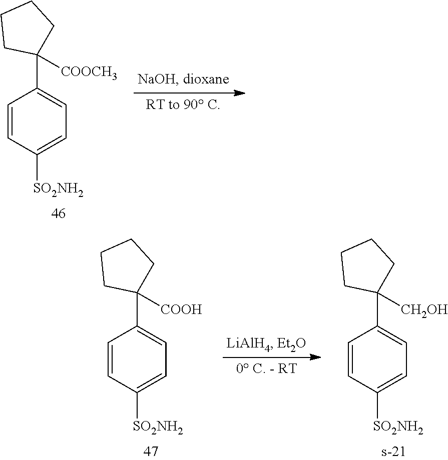 [SMILES: COC(=O)C1(c2ccc(S(N)(=O)=O)cc2)CCCC1 ; NS(=O)(=O)c1ccc(C2(C(=O)O)CCCC2)cc1 ; NS(=O)(=O)c1ccc(C2(CO)CCCC2)cc1]

Step 3: Synthesis of 47. To a solution of 46 m dioxane (4 mL) was added 4 M NaOH (8 mL) and heated at 90° C. rt overnight. The reaction mixture was diluted with 4M HCl and brine. The organic layer was dried and concentrated under vacuum. The crude product was purified with silica gel chromatography to obtain 47 (0.45 g).
Step 4: Synthesis of s-21. To a solution of 47 in Et₂O (10 mL) at 0° C. was added LiAlH₄ (2.0 eq.) and stirred at RT for 3 h. The reaction mixture was quenched with sat. NaOH/H₂O and washed with brine. The organic layer was dried and concentrated under vacuum. The crude product was purified with silica gel chromatography to obtain s-21 (0.25 g).

### Example 62. Synthesis of Compound 6-1

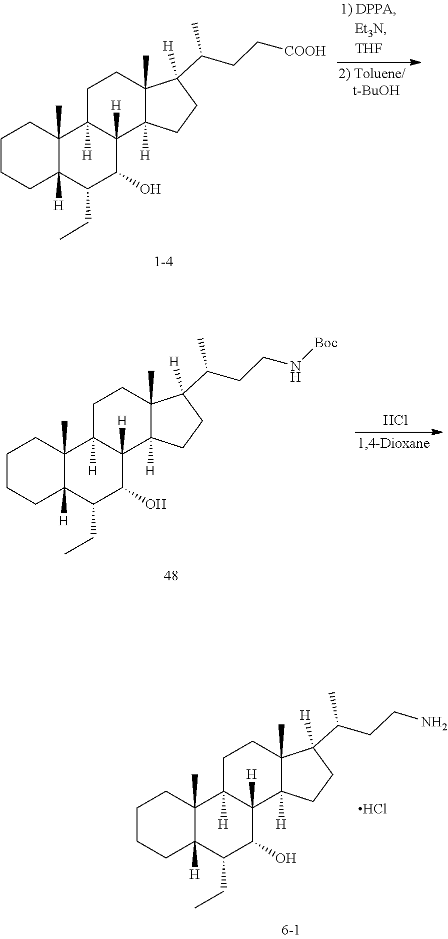

Step 1: Synthesis of Compound 48. The compound 1-4 (1.0 g, 2.47 mmol) was dissolved in THF (10.0 mL) and cooled to 0° C. To the solution was added Et₃N (0.5 g, 4.94 mmol) and DPPA (0.81 g, 2.97 mmol). The mixture was stirred at 0° C. for 4 h, quenched with brine and extracted with DCM (2*5 mL). The combined organic layer was dried over Na₂SO₄, filtered and concentrated in vacuo at 25° C. The crude obtained above was dissolved in toluene (10 ml), stirred at 100° C. for 30 min and t-BuOH (1.5 ml) was added. The mixture was stirred at 100° C. for 18 h, cooled to room temperature, diluted with EtOAc, and washed with water and brine. The organic layer was dried over Na₂SO₄, filtered and concentrated in vacuo. The crude product was purified by silica gel chromatography to give the desired product 48 (765 mg, 65% yield).
Step 2: Synthesis of Compound 6-1. To a solution of 48 (765 mg) in DCM (10 mL) was added HCl (4M in 1,4-Dioxane, 2.0 ml), and the mixture was stirred at room temperature 3 h. The solution was filtered and concentrated in vacuo. The crude product 6-1 (813 mg) was used for next step without purification.

### Example 63. Synthesis of Compound 6-2

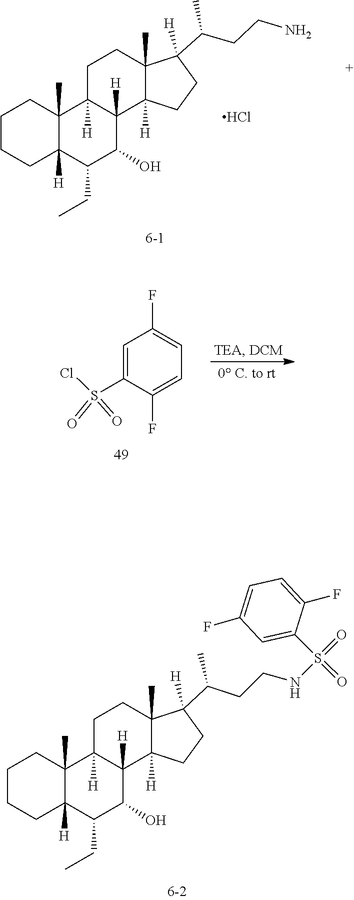

To a solution of 6-1 (50.0 mg, 0.121 mmol) in DCM (1 mL) was added TEA (36 mg, 0.363 mmol), and the mixture was cooled to 0° C. To the solution was added compound 49 (34 mg, 0.16 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 6-2 (44.4 mg, 66% yield). LC-MS, ES⁺ (m/z): 574.31 (M+23). ¹H NMR (400 MHz, Chloroform-d) δ 7.62 (m, 1H), 7.29-7.23 (m, 1H), 7.19 (t, J=9.0, 4.0 Hz, 1H), 4.75 (t, J=5.9 Hz, 1H), 3.71-3.64 (m, 1H), 3.16-2.92 (m, 2H), 1.95-1.86 (m, 1H), 1.81-0.83 (m, 35H), 0.62 (s, 3H).

### Example 64. Synthesis of Compound 6-3

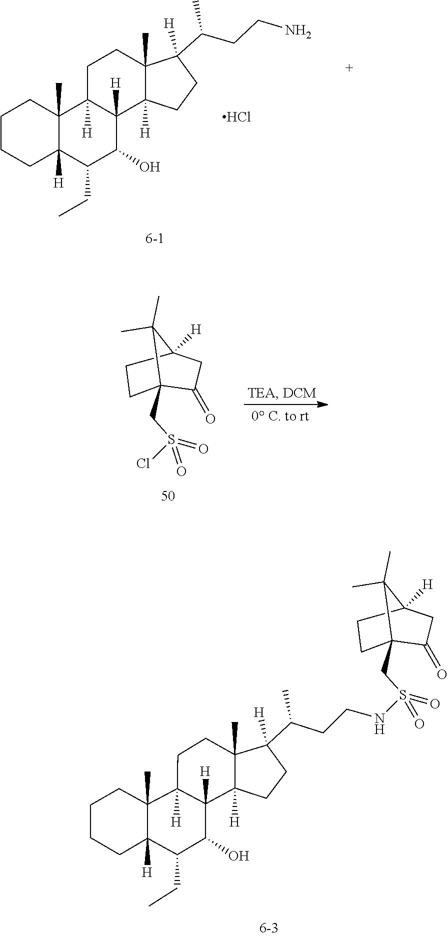

To a solution of 6-1 (50.0 mg, 0.121 mmol) in DCM (1 mL) was added TEA (36 mg, 0.363 mmol), and the mixture was cooled to 0° C. To the solution was added compound 50 (40 mg, 0.16 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 6-3 (60.3 mg, 85% yield). LC-MS, ES⁺ (m/z): 612.44 (M+23). ¹H NMR (400 MHz, Chloroform-d) δ5.10 (dd, J=7.4, 4.8 Hz, 1H), 3.68 (t, J=2.8 Hz, 1H), 3.39 (d, J=15.1 Hz, 1H), 3.23 (m, 1H), 3.06 (m, 1H), 2.89 (d, J=15.1 Hz, 1H), 2.38 (m, 1H), 2.23 (m, 1H), 2.12 (dd, J=5.0, 3.9 Hz, 1H), 2.07-1.09 (m, 31H), 1.02 (s, 3H), 0.95 (d, J=6.6 Hz, 3H), 0.92-0.85 (m, 9H), 0.65 (s, 3H).

### Example 65. Synthesis of Compound 6-4

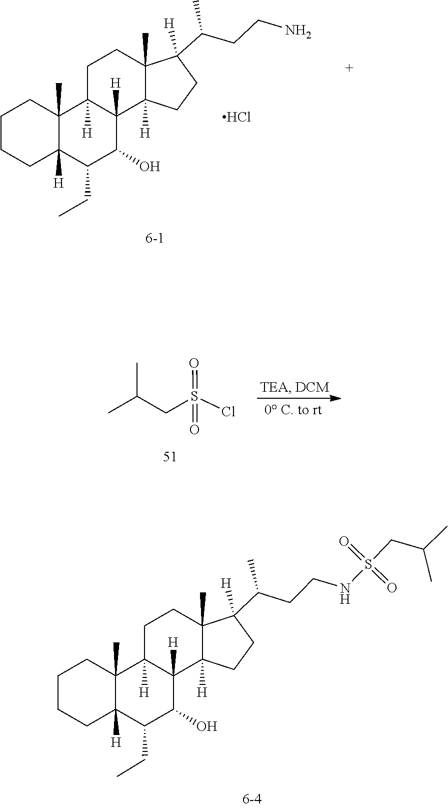

To a solution of 6-1 (50.0 mg, 0.121 mmol) in DCM (1 mL) was added TEA (36 mg, 0.363 mmol), and the mixture was cooled to 0° C. To the solution was added compound 51 (25 mg, 0.16 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 6-4 (32.3 mg, 54% yield). LC-MS, ES⁺ (m/z): 518.38 (M+23). ¹H NMR (400 MHz, Chloroform-d) δ 4.34 (t, J=6.1 Hz, 1H), 3.68 (t, J=2.8 Hz, 1H), 3.15 (m, 4.7 Hz, 1H), 3.03 (m, 1H), 2.89 (d, J=6.5 Hz, 2H), 2.24 (dp, J=13.3, 6.7 Hz, 1H), 1.98-1.12 (m, 27H), 1.10 (s, 3H), 1.09 (s, 3H), 0.94 (d, J=6.5 Hz, 3H), 0.90-0.86 (m, 6H), 0.65 (s, 3H).

### Example 66. Synthesis of Compound 6-5

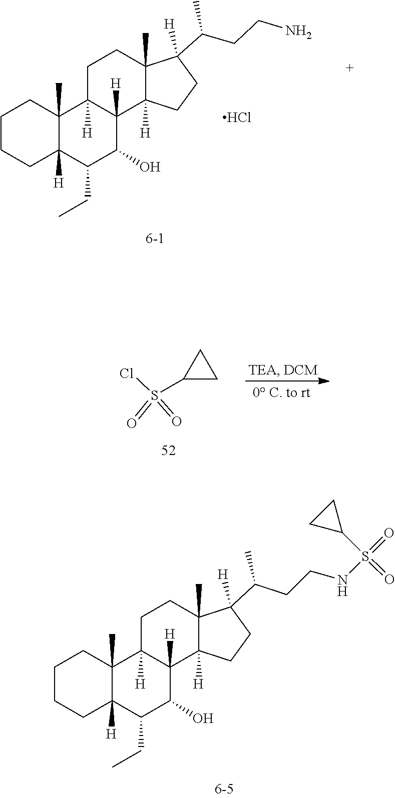

To a solution of 6-1 (50.0 mg, 0.121 mmol) in DCM (1 mL) was added TEA (36 mg, 0.363 mmol), and the mixture was cooled to 0° C. To the solution was added compound 52 (22.5 mg, 0.16 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 6-5 (17.4 mg, 29% yield). LC-MS, ES⁺ (m/z): 502.36 (M+23). ¹H NMR (400 MHz, Chloroform-d) δ 4.18 (t, J=6.1 Hz, 1H), 3.69 (t, J=2.8 Hz, 1H), 3.22 (m, 1H), 3.10 (m, 1H), 2.40 (tt, J=8.0, 4.8 Hz, 1H), 1.98-0.77 (m, 40H), 0.66 (s, 3H).

### Example 67. Synthesis of Compound 7-1

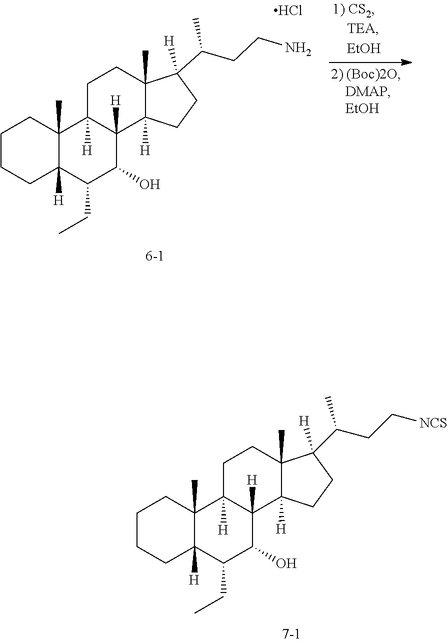

The 6-1 (300 mg, 0.728 mmol) was dissolved in EtOH (12 mL). To the solution was added TEA (735 mg, 7.28 mmol) and CS₂ (553 mg, 7.28 mmol). The mixture was stirred at room temperature for 3 h, cooled to −5° C. To the solution was added Boc₂O (159 mg, 0.728 mmol) and DMAP (9 mg, 0.073 mmol). The mixture was stirred at room temperature for overnight, quenched with brine (5 mL) and extracted with DCM (2*5 mL). The organic layer was dried over Na₂SO₄, filtered and concentrated in vacuo. The crude product was purified by silica gel chromatography to give the desired product 7-1 (291 mg, 96%).

### Example 68. Synthesis of Compound 7-2

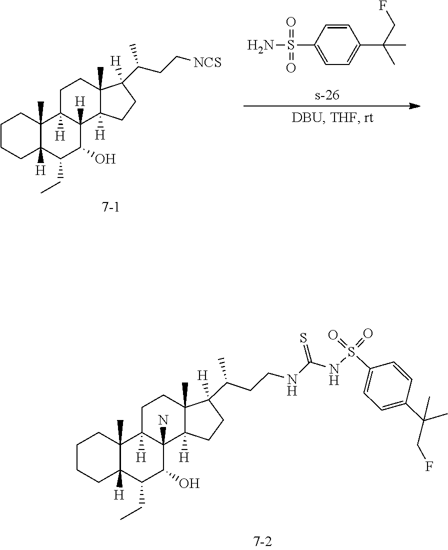

The compound s-26 (69 mg, 0.3 mmol) and DBU (46 mg, 0.3 mmol) in THF (1 ml) was added into a solution of the 7-1 (41.7 mg, 0.1 mmol) in toluene (1 ml). The mixture was stirred at RT for overnight. The mixture was quenched with water, extracted with EA, dried, filtered, and concentrated. The crude product was purified by silica gel chromatography to give the desired product 7-2 (40 mg, 61.7% yield). LC-MS, ES⁻ (m/z): 647.43 (M-1). ¹H NMR (400 MHz, Chloroform-d) δ 7.82-7.77 (m, 2H), 7.42 (d, J=8.1 Hz, 2H), 3.73-3.41 (m, 3H), 2.98 (d, J=21.6 Hz, 2H), 2.00-1.06 (m, 33H), 0.96 (d, J=6.6 Hz, 3H), 0.93-0.86 (m, 6H), 0.66 (s, 3H).

### Example 69. Synthesis of Compound 7-3

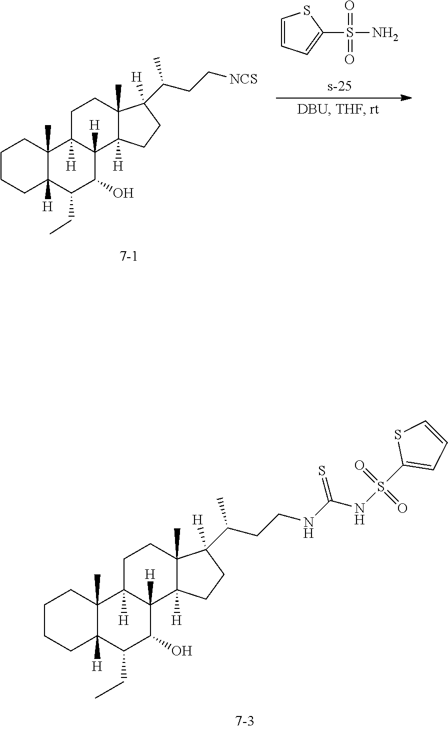

The compound s-25 (49 mg, 0.3 mmol) and DBU (46 mg, 0.3 mmol) in THF (1 ml) was added into a solution of the 7-1 (41.7 mg, 0.1 mmol) in toluene (1 ml). The mixture was stirred at RT for overnight. The mixture was quenched with water, extracted with EA, dried, filtered, and concentrated. The crude product was purified by silica gel chromatography to give the desired product 7-3. LC-MS, ES⁻ (m/z): 579.30 (M-1). ¹H NMR (400 MHz, Chloroform-d) δ 8.08 (s, 1H), 7.73-7.69 (m, 2H), 7.15 (dd, J=4.9, 4.0 Hz, 1H), 3.72-3.47 (m, 3H), 2.02-1.07 (m, 27H), 0.99 (d, J=6.5 Hz, 3H), 0.93-0.87 (m, 6H), 0.67 (s, 3H).

### Example 70. Synthesis of Compound 8-1

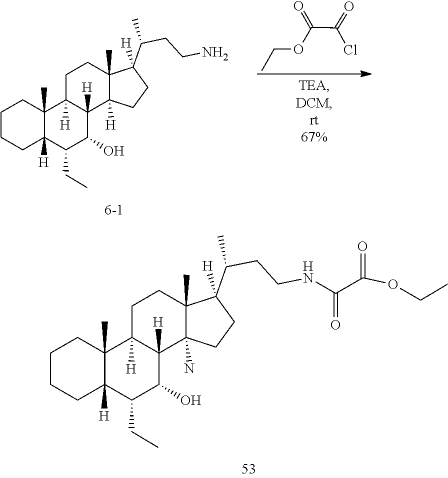

Step 1: synthesis of 53. To a solution of 6-1 (300 mg, 0.8 mmol) in DCM (6 mL) was added TEA (242 mg, 2.4 mmol) and ethyl oxalyl monochloride (131 mg, 0.96 mmol) at 0° C. The mixture was stirred at room temperature for one hour. After TLC indicated complete reaction, the solution was quenched by adding 5 mL water, and the mixture was extracted with DCM. The combined organic layers were dried over anhydrous Na₂SO₄, filtered, and concentrated in vacuum. The crude product was purified by silica gel chromatography to give the 53 (255.0 mg, 67% yield). LC-MS, ES⁻ (m/z): 476.37 (M+1). ¹H NMR (400 MHz, CDCl₃) δ 7.02 (s, 1H), 4.40-4.28 (m, 2H), 3.67 (s, 1H), 3.47-3.20 (m, 2H), 1.97-1.85 (m, 2H), 1.80-1.72 (m, 2H), 1.71-1.58 (m, 5H), 1.53-1.28 (m, 12H), 1.26-1.06 (m, 8H), 1.01-0.94 (m, 4H), 0.93-0.84 (m, 7H), 0.65 (s, 3H).

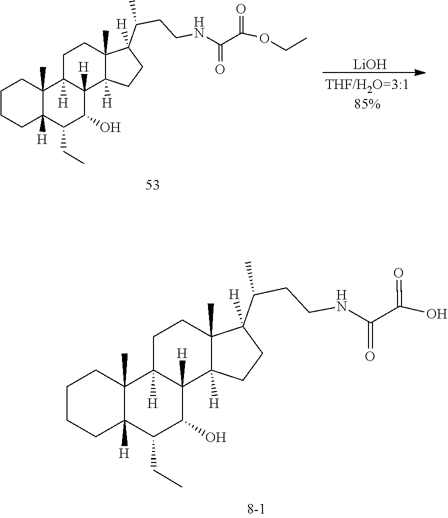

Step 2: synthesis of 8-1. To a solution of compound 53 (210 mg, 0.44 mmol) in THF (6 mL) and H₂O (2 mL) was added LiOH (53 mg, 2.2 mmol), and the mixture was stirred at room temperature overnight. After TLC indicated complete reaction, the reaction mixture was acidified with 1M HCl. Solvent was removed, and the residue was extracted with EA. The combined organic layers were dried over anhydrous Na₂SO₄, filtered, and concentrated in vacuum to give the desired product 8-1 (168.0 mg, 85% yield). LC-MS, ES⁻ (m/z): 446.33 (M-1). ¹H NMR (400 MHz, CDCl₃) δ 3.68 (s, 1H), 3.46-3.24 (m, 2H), 1.97-1.59 (m, 8H), 1.53-1.37 (m, 6H), 1.37-1.26 (m, 4H), 1.25-1.12 (m, 8H), 1.01-0.94 (m, 3H), 0.93-0.83 (m, 8H), 0.65 (s, 3H).

### Example 71. Synthesis of Compound 8-2

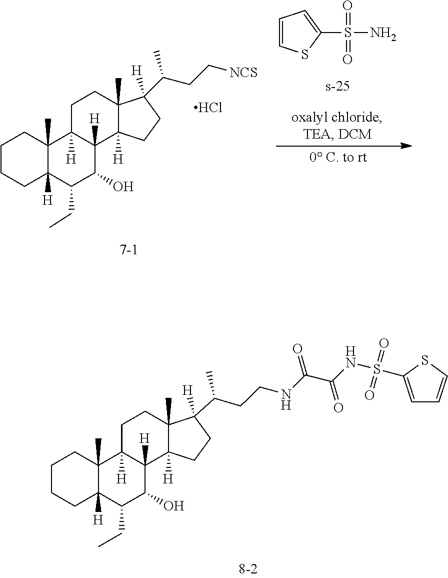

To a solution of 6-1 (50.0 mg, 0.121 mmol) in DCM (2 mL) was added TEA (49 mg, 0.484 mmol), B (20 mg, 0.121 mmol) and the mixture was cooled to 0° C. To the solution was added oxalyl chloride (17 mg, 0.133 mmol). The mixture was stirred at room temperature for 3 h, quenched with brine and extracted with DCM (2*5 mL). The combined organic layer was dried over Na₂SO₄, filtered and concentrated in vacuo. The crude product was purified by silica gel chromatography to give the desired product 8-2 (9 mg, 13%). LC-MS, ES⁻ (m/z): 591.31 (M-1). ¹H NMR (400 MHz, Chloroform-d) δ 7.72-7.49 (m, 1H), 7.34 (m, 1H), 6.92 (m, 1H), 3.66 (s, 1H), 3.37-2.97 (m, 2H), 1.93-1.03 (m, 27H), 0.91-0.80 (m, 6H), 0.60 (s, 3H).

### Example 72. Synthesis of Compound 8-3

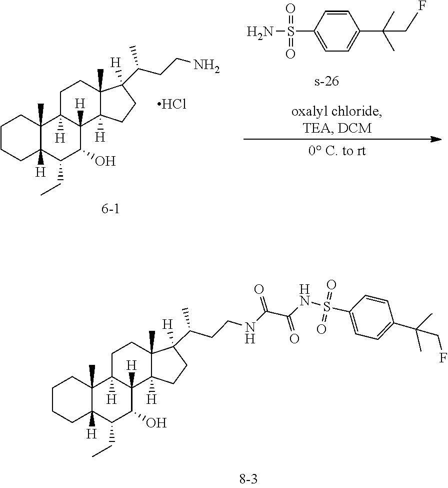

To a solution of 6-1 (50.0 mg, 0.121 mmol) in DCM (2 mL) was added TEA (49 mg, 0.484 mmol), s-26 (28 mg, 0.121 mmol) and the mixture was cooled to 0° C. To the solution was added oxalyl chloride (17 mg, 0.133 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 8-3 (25 mg, 31%). LC-MS, ES⁻ (m/z): 659.44 (M-1). ¹H NMR (400 MHz, Chloroform-d) δ 7.90 (s, 2H), 7.26 (s, 2H), 3.66 (s, 1H), 3.34-3.02 (m, 2H), 2.89 (d, J=20.8 Hz, 2H), 1.98-0.99 (m, 33H), 0.93-0.80 (m, 9H), 0.59 (s, 3H).

### Example 73. Synthesis of Compound 8-4

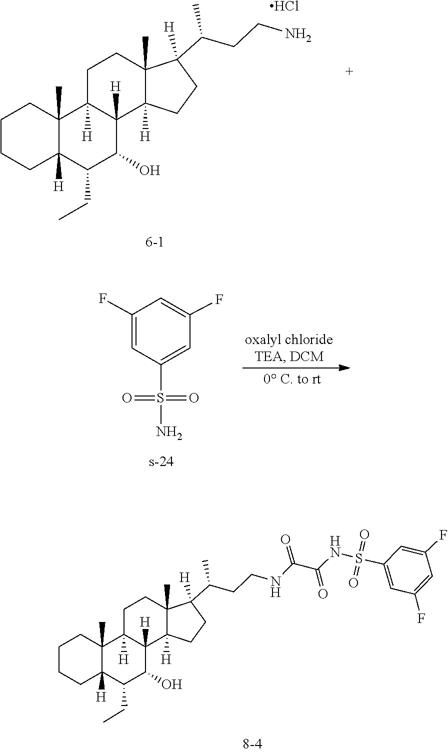

To a solution of 6-1 (50.0 mg, 0.121 mmol) in DCM (2 mL) was added TEA (49 mg, 0.484 mmol), s-24 (23 mg, 0.121 mmol) and the mixture was cooled to 0° C. To the solution was added oxalyl chloride (17 mg, 0.133 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 8-4. LC-MS, ES⁻ (m/z): 621.35 (M-1). H NMR (400 MHz, Chloroform-d) δ 7.65-7.49 (m, 1H), 7.43 (s, 2H), 6.88 (s, 1H), 3.65 (t, J=2.8 Hz, 1H), 3.36-3.03 (m, 2H), 2.27-0.99 (m, 27H), 0.88 (m, 9H), 0.58 (s, 3H).

### Example 74. Synthesis of Compound 8-5

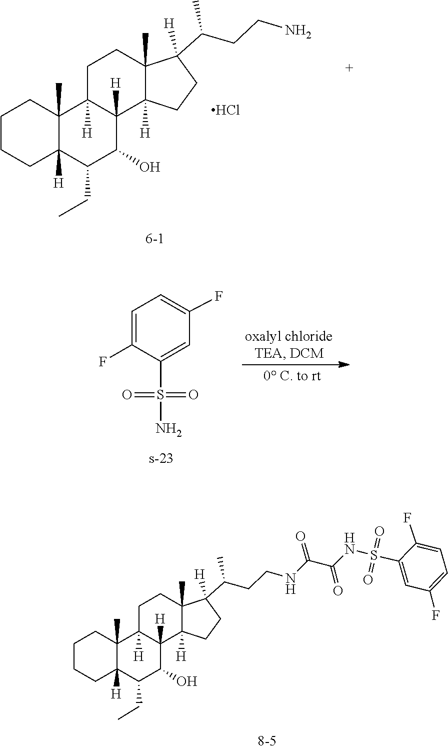

To a solution of 6-1 (50.0 mg, 0.121 mmol) in DCM (2 mL) was added TEA (49 mg, 0.484 mmol), s-23 (23 mg, 0.121 mmol) and the mixture was cooled to 0° C. To the solution was added oxalyl chloride (17 mg, 0.133 mmol). The mixture was stirred at room temperature for 3 h, quenched with brine (5 mL) and extracted with DCM (2*5 mL). The combined organic layer was dried over Na₂SO₄, filtered and concentrated in vacuo. The crude product was purified by silica gel chromatography to give the desired product 8-5. LC-MS, ES⁻ (m/z): 621.32 (M-1). ¹H NMR (400 MHz, Chloroform-d) δ 7.68-7.47 (m, 2H), 7.16-6.97 (m, 1H), 3.65 (s, 1H), 3.37-3.09 (m, 2H), 1.99-1.02 (m, 27H), 0.94-0.84 (m, 9H), 0.60 (s, 3H).

### Example 75. Synthesis of Compound 9-1

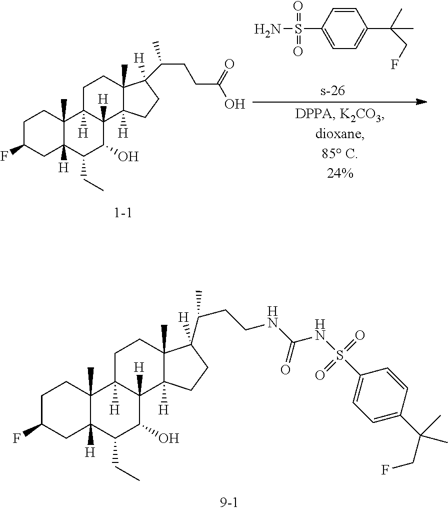

Into an 8 mL vial were added 1-1 (50.0 mg, 0.11 mmol) and 1,4-dioxane (2 mL) at room temperature. 4-(1-fluoro-2-methylpropan-2-yl)-benzene-sulfonamide s-26 (27.0 mg, 0.11 mmol), DPPA (39.0 mg, 0.14 mmol) and K₂CO₃ (50 mg, 0.36 mmol) was added to the above solution under Argon atmosphere. The resulting mixture was stirred at 85° C. for 3 h. The reaction was monitored by TLC. The resulting mixture was filtered, filtrate was concentrated under reduced pressure. The residue was purified by silica gel column chromatography to obtain the 9-1 (18.7 mg, 24% yield). LC-MS, ES⁻ (m/z): 649.39 (M-1). ¹H NMR (400 MHz, CDCl₃) δ 7.79 (d, 2H), 7.38 (d, 2H), 6.50 (s, 1H), 4.91-4.72 (d, 1H), 3.72 (s, 1H), 3.37-3.11 (m, 2H), 3.04-2.90 (d, 2H), 2.23-1.81 (m, 5H), 1.80-1.60 (m, 5H), 1.50-1.34 (m, 11H), 1.23-1.09 (m, 7H), 0.97-0.89 (m, 10H), 0.88-0.84 (m, 3H), 0.65 (s, 3H).

### Example 76. Synthesis of Compound 9-2

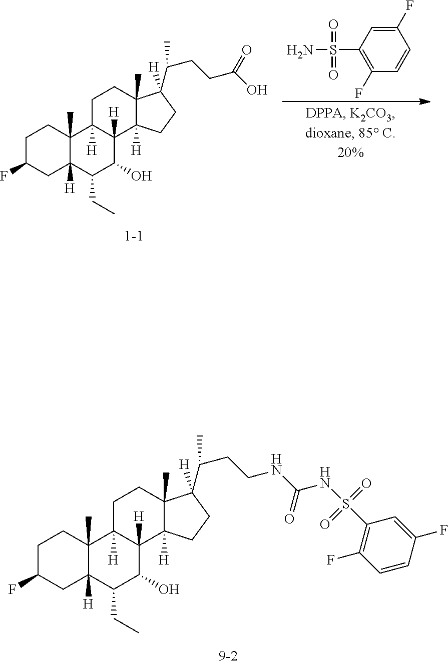

Into an 8 mL vial were added 1-1 (50.0 mg, 0.12 mmol) and 1,4-dioxane (2 mL) at room temperature. 2,5-difluorobenzenesulfonamide s-23 (23.0 mg, 0.12 mmol), DPPA (38.0 mg, 0.14 mmol) and K₂CO₃ (50 mg, 0.36 mmol) was added to the above solution under Argon atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the 9-2 (14.7 mg, 20% yield). LC-MS, ES⁻ (m/z): 611.32 (M-1). ¹H NMR (400 MHz, CDCl₃) δ 7.64-7.49 (m, 1H), 7.38-7.28 (m, 1H), 7.22-7.08 (m, 1H), 6.38 (s, 1H), 4.90-4.72 (d, 1H), 3.71 (s, 1H), 3.36-3.08 (m, 2H), 2.23-1.77 (m, 6H), 1.71-1.53 (m, 9H), 1.51-1.34 (m, 10H), 0.95-0.89 (m, 8H), 0.87-0.81 (m, 2H), 0.64 (s, 3H).

### Example 77. Synthesis of Compound 9-3

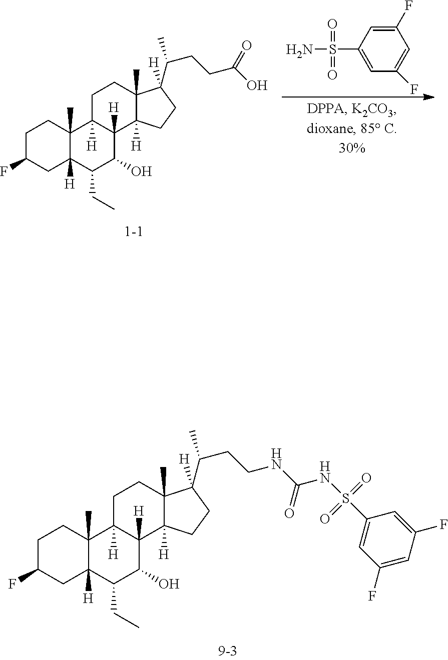

Into an 8 mL vial were added 1-1 (50.0 mg, 0.12 mmol) and 1,4-dioxane (2 mL) at room temperature. 3,5-difluorobenzenesulfonamide (23.0 mg, 0.12 mmol), DPPA (38.0 mg, 0.14 mmol) and K₂CO₃ (50 mg, 0.36 mmol) was added to the above solution under Argon atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the compound 9-3 (22.2 mg, 30% yield). LC-MS, ES⁻ (m/z): 611.32 (M-1). ¹H NMR (400 MHz, CDCl₃) δ 7.46-7.39 (m, 2H), 7.14-7.05 (m, 1H), 6.41 (s, 1H), 4.93-4.71 (d, 1H), 3.70 (s, 1H), 3.38-3.10 (m, 2H), 2.28-1.80 (m, 4H), 1.76-1.65 (m, 3H), 1.52-1.34 (m, 9H), 1.26-1.09 (m, 9H), 0.98-0.83 (m, 10H), 0.65 (s, 3H).

### Example 78. Synthesis of Compound 9-4

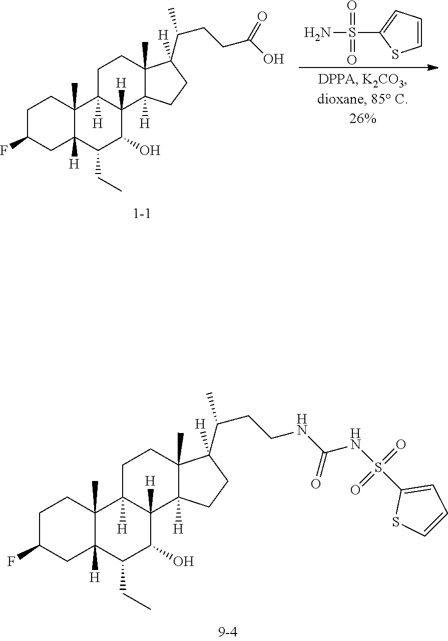

Into an 8 mL vial were added 1-1 (50.0 mg, 0.12 mmol) and 1,4-dioxane (2 mL) at room temperature. Thiophene-2-sulfonamide (19.0 mg, 0.12 mmol), DPPA (38.0 mg, 0.14 mmol) and K₂CO₃ (50 mg, 0.36 mmol) was added to the above solution under Ar atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the compound 9-4 (18.1 mg, 26% yield). LC-MS, ES⁻ (m/z): 581.30 (M-1). ¹H NMR (400 MHz, CDCl₃) δ 7.71-7.66 (m, 1H), 7.65-7.60 (m, 1H), 7.13-7.04 (m, 1H), 6.46 (s, 1H), 4.91-4.71 (d, 1H), 3.36-3.24 (m, 1H), 3.22-3.09 (m, 1H), 2.27-1.92 (m, 2H), 1.91-1.78 (m, 2H), 1.74-1.53 (m, 7H), 1.51-1.36 (m, 7H), 1.31 (s, 2H), 1.23-1.07 (m, 6H), 0.97-0.84 (m, 10H), 0.65 (s, 3H).

### Example 79. Synthesis of Compound 9-5

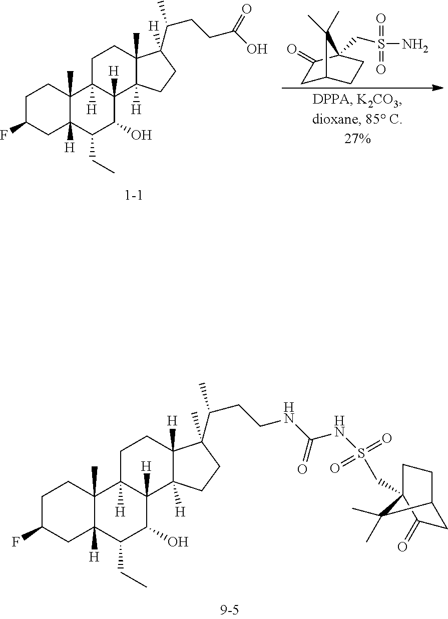

Into an 8 mL vial were added 1-1 (45.0 mg, 0.11 mmol) and 1,4-dioxane (2 mL) at room temperature. ((1S,4R)-7,7-dimethyl-2-oxobicyclo-[2.2.1]-heptan-1-yl)-methane-sulfonamide s-27 (25.0 mg, 0.11 mmol), DPPA (36.0 mg, 0.13 mmol) and K₂CO₃ (46 mg, 0.33 mmol) was added to the above solution under Ar₂ atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the compound 9-5 (17.5 mg, 27% yield). LC-MS, ES⁻ (m/z): 651.41 (M+1). ¹H NMR (400 MHz, CDCl₃) δ 6.49 (s, 1H), 4.92-4.69 (d, 1H), 3.72-3.68 (m, 1H), 3.68-3.59 (d, 2H), 3.42-3.00 (m, 2H), 2.45-2.35 (m, 1H), 2.27-1.80 (m, 8H), 1.52-1.35 (m, 9H), 1.33-1.28 (m, 4H), 1.25-1.08 (m, 11H), 1.04 (s, 2H), 0.99-0.88 (m, 11H), 0.87-0.84 (m, 3H), 0.65 (s, 3H).

### Example 80. Synthesis of Compound 9-6

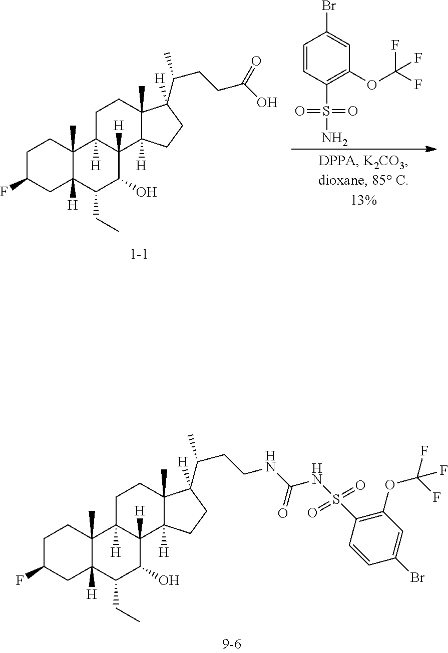

Into an 8 mL vial were added 1-1 (53.0 mg, 0.12 mmol) and 1,4-dioxane (2 mL) at room temperature. 4-bromo-2-(trifluoro-methoxy)-benzene-sulfonamide s-28 (40.0 mg, 0.12 mmol), DPPA (41.0 mg, 0.15 mmol) and K₂CO₃ (52 mg, 0.37 mmol) was added to the above solution under Argon atmosphere. The resulting mixture was stirred 3 h at 85° C. The reaction was monitored by TLC. The resulting mixture was filtered. The filtrate was concentrated under reduced pressure. The residue was purified by silica gel column chromatography to obtain the 9-6 (14.5 mg, 13% yield). LC-MS, ES⁻ (m/z): 739.23 (M+1). ¹H NMR (400 MHz, CDCl₃) δ 7.92-7.87 (d, 1H), 7.65-7.49 (m, 2H), 6.29 (s, 1H), 4.89-4.72 (d, 1H), 3.71 (s, 1H), 3.29-3.19 (m, 1H), 3.17-3.05 (m, 1H), 2.31-1.78 (m, 3H), 1.74-1.51 (m, 7H), 1.34-1.30 (s, 2H), 1.28-1.26 (s, 2H), 1.23-1.07 (m, 6H), 0.99-0.81 (m, 9H), 0.67 (s, 3H).

### Example 81. Synthesis of Compound 9-7

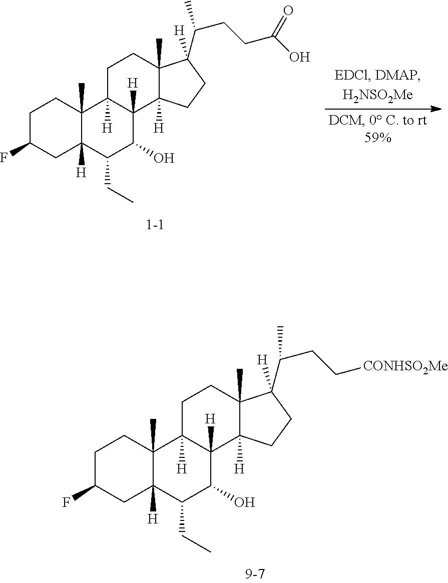

To a solution of EDCI (1.4 mg) in DCM (1 mL) was added DMAP (1.6 mg), and the mixture was stirred at room temperature for 30 min. The mixture was cooled to 0° C., followed by addition of a solution of H₂NSO₂Me (0.5 mg) and compound 1-1 (2.3 mg) in DCM (1 mL), and the mixture was stirred at room temperature overnight. Then the mixture was quenched with saturated NaHCO₃ solution, extracted with EA, and the combined organic layers were dried over anhydrous Na₂SO₄, filtered, and concentrated in vacuo. The crude product was purified by silica gel chromatography to give the desired product 9-7 (1.6 mg, 59%). H NMR (400 MHz, Chloroform-d) δ 4.82 (d, J=44.9 Hz, 1H), 3.72 (s, 1H), 0.94-0.89 (m, 9H), 0.67 (s, 3H).

### Example 82. Synthesis of Compound 10-1

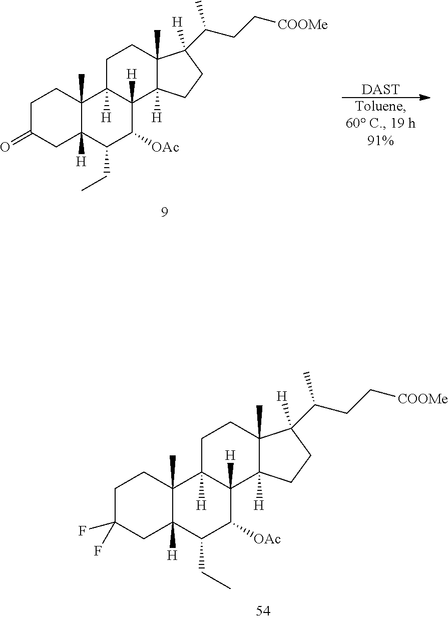

Step 1: Synthesis of Compound 54. To a solution of 9 (1.0 g, 2.1 mmol) in toluene (10 mL) was added DAST (3.38 g, 21 mmol) at ice bath. The mixture was stirred at 60° C. overnight. After TLC indicated complete reaction, the solvent is evaporated under reduced pressure, followed by addition of saturated NaHCO₃ solution, and the mixture was extracted with EA. The combined organic layers were dried over anhydrous Na₂SO₄, filtered, and concentrated under reduced pressure. The crude product was purified by silica gel chromatography to give the compound 54 (952.0 mg, 91% yield). LC-MS, ES⁻ (m/z): 495.34 (M-1). ¹H NMR (400 MHz, CDCl₃) δ 5.11 (s, 1H), 3.65 (s, 3H), 2.39-2.29 (m, 1H), 2.27-2.08 (m, 1H), 2.06 (s, 3H), 1.99-1.94 (m, 1H), 1.87-1.71 (m, 6H), 1.69-1.61 (m, 2H), 1.53-1.21 (m, 10H), 1.19-1.00 (m, 5H), 0.96 (s, 3H), 0.93-0.81 (m, 7H), 0.64 (s, 3H).

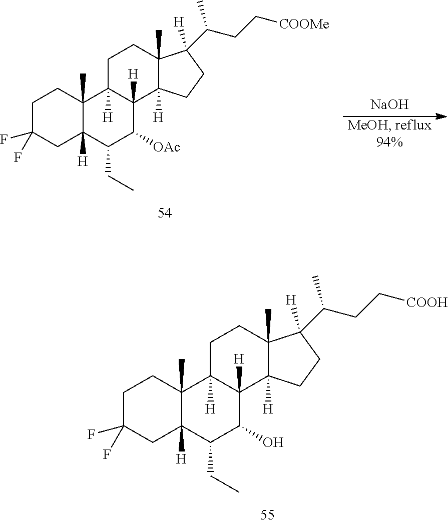

Step 2: Synthesis of Compound 55. To a solution of 54 (500.0 mg, 1.0 mmol) in CH₃OH (60 mL) was added NaOH (13.6 g, 340 mmol), and the mixture was refluxed overnight. After cooling to room temperature, the reaction mixture was acidified with 4 M HCl. Then the solvent was removed, and the residue was extracted with EA. The combined organic layers were dried over anhydrous Na₂SO₄, filtered, and concentrated in vacuum. The crude product was purified by silica gel chromatography to give the desired product 55 (417.0 mg, 94% yield). LC-MS, ES⁻ (m/z): 439.31 (M-1). ¹H NMR (400 MHz, CDCl₃) δ 3.71 (s, 1H), 2.49-2.33 (m, 2H), 2.31-2.20 (m, 1H), 2.09-1.64 (m, 9H), 1.57-1.25 (m, 13H), 1.21-1.07 (m, 3H), 0.97-0.85 (m, 9H), 0.66 (s, 3H).

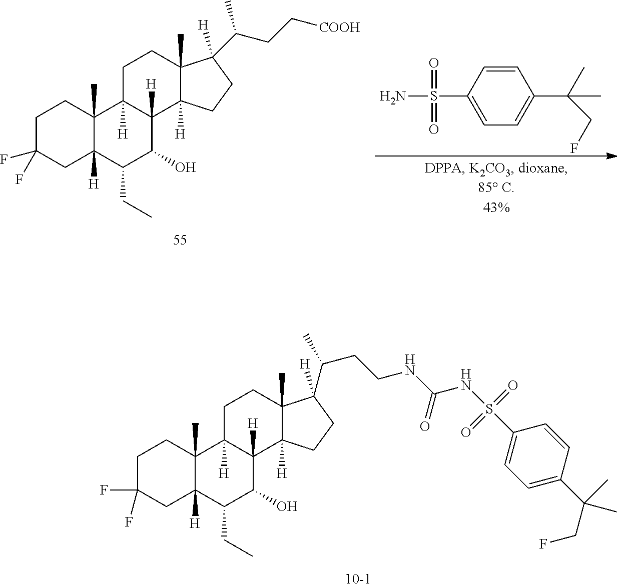

Step 3: Synthesis of Compound 10-1. Into an 8 mL vial were added 55 (50.0 mg, 0.11 mmol) and 1,4-dioxane (2 mL) at room temperature. 4-(1-fluoro-2-methylpropan-2-yl)-benzene-sulfonamide (26.0 mg, 0.11 mmol), DPPA (37.4 mg, 0.13 mmol) and K₂CO₃ (45 mg, 0.33 mmol) was added to the above solution under Ar₂ atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the 10-1 (32.0 mg, 43% yield). LC-MS, ES⁻ (m/z): 667.38 (M-1). ¹H NMR (400 MHz, CDCl₃) δ 7.80 (d, 2H), 7.40 (d, 2H), 6.53 (s, 1H), 3.72 (s, 1H), 3.39-3.08 (m, 2H), 3.05-2.90 (d, 2H), 1.98-1.66 (m, 8H), 1.51-1.39 (m, 7H), 1.39-1.34 (m, 4H), 1.31-1.29 (m, 4H), 1.25-1.12 (m, 9H), 0.99-0.82 (m, 9H), 0.66 (s, 3H).

### Example 83. Synthesis of Compound 10-2

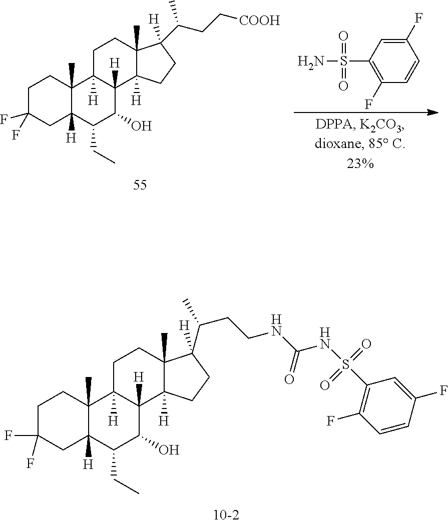

Into an 8 mL vial were added 55 (50.0 mg, 0.11 mmol) and 1,4-dioxane (2 mL) at room temperature. 2,5-difluorobenzenesulfonamide (22.0 mg, 0.11 mmol), DPPA (36.0 mg, 0.13 mmol) and K₂CO₃ (45 mg, 0.33 mmol) was added to the above solution under Argon atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the 10-2 (16.3 mg, 23% yield). LC-MS, ES⁻ (m/z): 629.31 (M-1). ¹H NMR (400 MHz, CDCl₃) δ 7.63-7.58 (m, 1H), 7.38-7.30 (m, 1H), 7.25-7.20 (m, 1H), 6.38 (s, 1H), 3.71 (s, 1H), 3.35-3.08 (m, 2H), 2.44-1.93 (m, 2H), 1.90-1.66 (m, 5H), 1.48-1.36 (m, 7H), 1.23-1.10 (m, 8H), 0.98-0.89 (m, 9H), 0.87-0.80 (m, 4H), 0.64 (s, 3H).

### Example 84. Synthesis of Compound 10-3

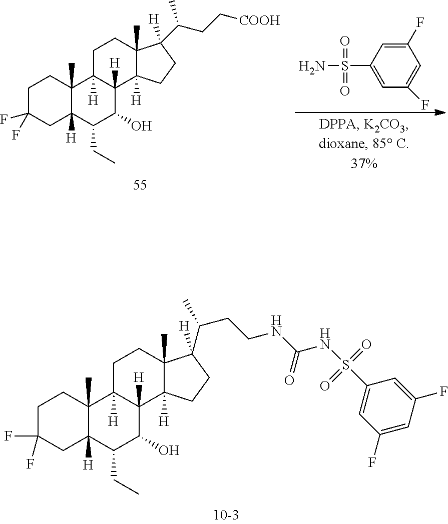

Into an 8 mL vial were added 55 (50.0 mg, 0.11 mmol) and 1,4-dioxane (2 mL) at room temperature. 3,5-difluorobenzenesulfonamide (22.0 mg, 0.11 mmol), DPPA (36.0 mg, 0.13 mmol) and K₂CO₃ (45 mg, 0.33 mmol) was added to the above solution under Argon atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the 10-3 (26.2 mg, 37% yield). LC-MS, ES⁻ (m/z): 629.31 (M-1). ¹H NMR (400 MHz, CDCl₃) δ 7.45-7.40 (m, 2H), 7.15-7.05 (m, 1H), 6.45 (s, 1H), 3.72 (s, 1H), 3.37-3.25 (m, 1H), 3.23-3.12 (m, 1H), 2.49-2.19 (m, 1H), 2.15-2.02 (m, 1H), 2.02-1.73 (m, 5H), 1.51-1.37 (m, 7H), 1.35-1.30 (s, 4H), 1.22-1.13 (m, 4H), 1.00-0.80 (m, 4H), 0.65 (s, 3H).

Example 85. Synthesis of Compound 10-4

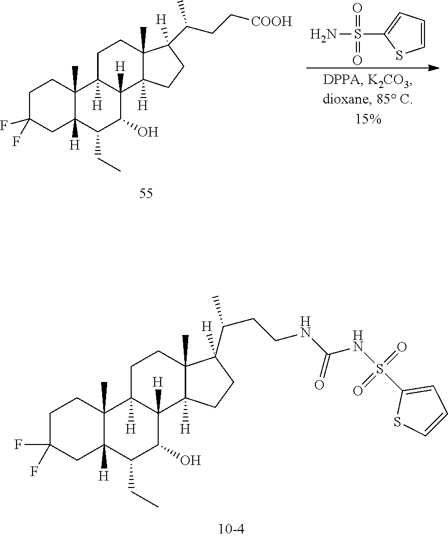

Into an 8 mL vial were added 55 (50.0 mg, 0.1 mmol) and 1,4-dioxane (2 mL) at room temperature, thiophene-2-sulfonamide (18.0 mg, 0.11 mmol), DPPA (35.0 mg, 0.13 mmol) and K₂CO₃ (45 mg, 0.33 mmol) was added to the above solution under Argon atmosphere. The resulting mixture was stirred 3 h at 85° C. The reaction was monitored by TLC. The resulting mixture was filtered, filtrate was concentrated under reduced pressure. The residue was purified by silica gel column chromatography to obtain the 10-4 (9.6 mg, 15% yield). LC-MS, ES⁻ (m/z): 601.29 (M+1). ¹H NMR (400 MHz, CDCl₃) δ 7.70-7.66 (m, 2H), 7.15-7.09 (m, 1H), 6.48 (s, 1H), 3.71 (s, 1H), 3.37-3.28 (m, 1H), 3.24-3.13 (m, 1H), 2.49-2.16 (m, 1H), 2.04-1.68 (m, 9H), 1.50-1.36 (m, 10H), 1.22-1.10 (m, 7H), 0.97-0.92 (m, 8H), 0.65 (s, 3H).

### Example 86. Synthesis of 11-1

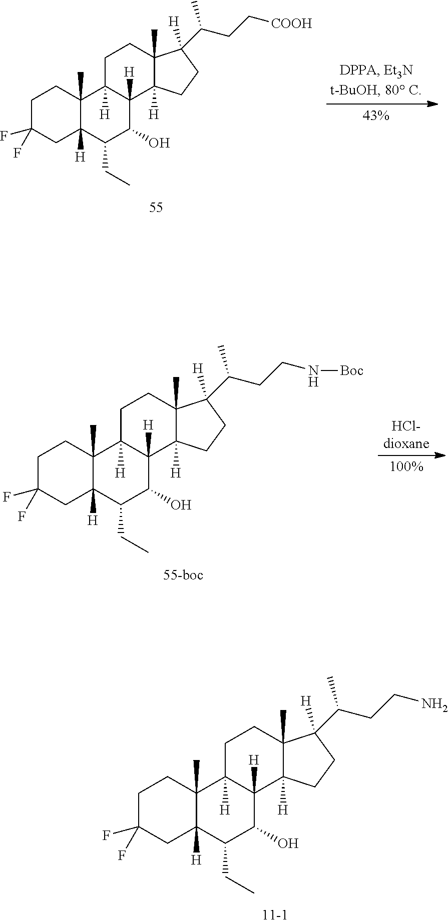

Step 1: Synthesis of Compound 55-boc. Compound 55 (450 mg, 1.02 mmol) was dissolved in t-BuOH (10 mL). Crushed molecular sieves (4A) were added and the reaction mixture was stirred for 30 min under an argon atmosphere. Et₃N (206 mg, 2.04 mmol) and DPPA (337 mg, 1.22 mmol) were added and the reaction mixture was heated under reflux. After 4 h the solution was filtered and concentrated in vacuum, the crude product was purified by silica gel chromatography to give the compound 55-boc (255.0 mg, 67% yield). LC-MS, ES⁻ (m/z): 510.38 (M-1). ¹H NMR (400 MHz, CDCl₃) δ 3.71 (s, 1H), 3.36-2.93 (m, 2H), 2.53-2.28 (m, 1H), 2.02-1.64 (m, 6H), 1.50-1.09 (m, 25H), 1.00-0.74 (m, 12H), 0.65 (s, 3H).
Step 2: synthesis of 11-1. Into a 40 mL vial were added compound 55-boc (224.0 mg, 0.44 mmol) and HCl-dioxane (5 mL) at room temperature. A mixture was stirred for 2 h at room temperature under air atmosphere. The reaction was monitored by TLC. The resulting mixture was concentrated under vacuum. This resulted in compound II-1 (238 mg, 100% yield) as a white solid. LC-MS, ES⁻ (m/z): 410.33 (M-1). ¹H NMR (400 MHz, CDCl₃) δ 3.37-3.11 (m, 1H), 2.49-2.29 (m, 1H), 2.04-1.85 (m, 3H), 1.84-1.75 (m, 3H), 1.73-1.62 (m, 5H), 1.56-1.53 (m, 2H), 1.51-1.33 (m, 7H), 1.25-1.14 (m, 5H), 1.00-0.78 (m, 10H), 0.72-0.61 (m, 3H).

### Example 87. Synthesis of 11-2

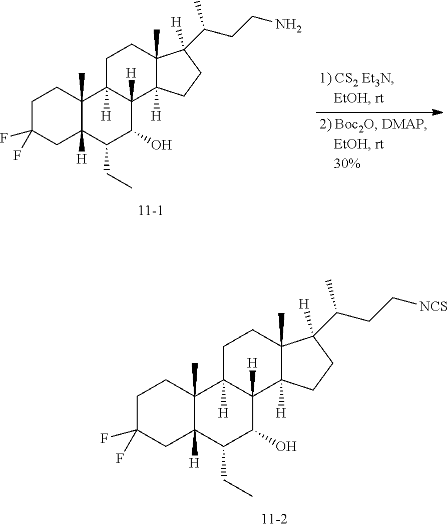

To a solution of compound II-1 (238 mg, 0.58 mmol) in anhydrous ethanol was added CS₂ (441 mg, 5.8 mmol) and triethyl amine (586 mg, 5.8 mmol). The mixture was stirred at room temperature until intermediate was formed. Then, the reaction was cooled in an ice bath at −5° C. Di-tert-butyl di-carbonate (Boc₂O) (126 mg, 0.58 mmol) and DMAP (2 mg, 0.017 mmol) was dissolved in 0.5 mL anhydrous ethanol, separately. At first, di-tert-butyl di-carbonate solution was added to the reaction mixture, and then the addition of DMAP solution was performed immediately. The reaction was kept in an ice bath for 5 min and then was allowed to reach room temperature. The reaction was monitored by TLC. The reaction mixture was concentrated under vacuum and 10 mL water was added to residue. The mixture was extracted with chloroform (3×20 mL) and organic phases were collected, dried over anhydrous Na₂SO₄ and concentrated under vacuum. The products were purified by silica gel chromatography to give the compound II-2 (80.0 mg, 30% yield). LC-MS, ES⁻ (m/z): 452.29 (M-1). ¹H NMR (400 MHz, CDCl₃) δ 3.72 (s, 1H), 3.64-3.40 (m, 2H), 2.56-2.30 (m, 1H), 2.06-1.68 (m, 9H), 1.51-1.39 (m, 5H), 1.38-1.27 (m, 6H), 1.22-1.11 (m, 4H), 1.05-0.80 (m, 10H), 0.72-0.65 (m, 3H).

### Example 88. Synthesis of Compound 11-3

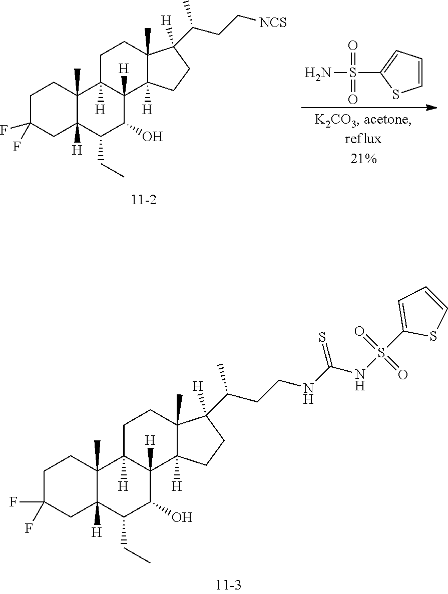

Into an 8 mL vial were added compound II-2 (34.0 mg, 0.075 mmol) and acetone (2 mL) at room temperature, thiophene-2-sulfonamide (10.0 mg, 0.062 mmol), K₂CO₃ (21 mg, 0.15 mmol) was added to the above solution under Argon atmosphere. The resulting mixture was stirred 3 h at 60° C. The reaction was monitored by TLC. The resulting mixture was filtered, filtrate was concentrated under reduced pressure. The residue was purified by silica gel column chromatography to obtain the 11-3 (8.2 mg, 21% yield). LC-MS, ES⁻ (m/z): 615.26 (M-1). ¹H NMR (400 MHz, CDCl₃) δ 7.72-7.65 (m, 1H), 7.63-7.47 (m, 1H), 7.14-7.03 (m, 1H), 3.71 (s, 1H), 3.59-3.31 (m, 1H), 2.49-2.16 (m, 1H), 2.05-1.72 (m, 7H), 1.51-1.35 (m, 10H), 1.01-0.77 (m, 18H), 0.70-0.58 (m, 3H).

### Example 89. Synthesis of Compound 11-4

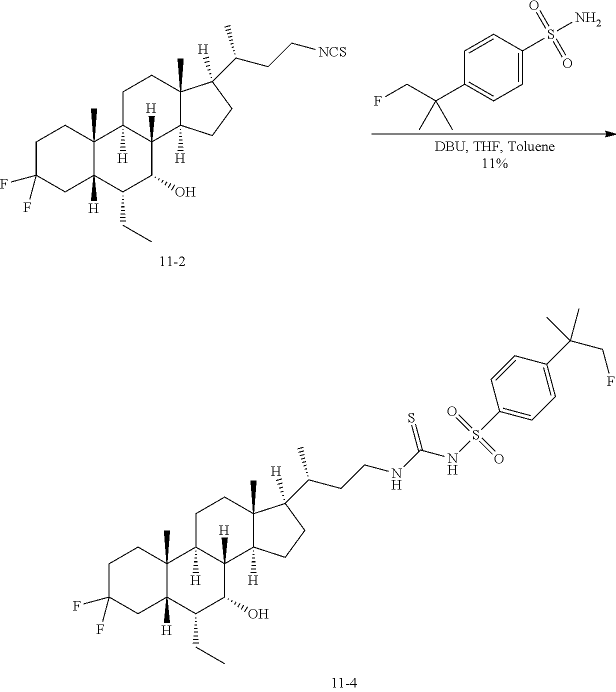

Into an 8 mL vial were added 11-2 (31.7 mg, 0.07 mmol) and toluene (1 mL) at room temperature. 4-(1-fluoro-2-methylpropan-2-yl)-benzene-sulfonamide (32.4 mg, 0.14 mmol), DBU (32 mg, 0.21 mmol) and THF (1 mL) was added to the above solution under air atmosphere. The resulting mixture was stirred overnight at room temperature. The reaction was monitored by TLC. The resulting mixture was concentrated under reduced pressure. The residue was purified by silica gel column chromatography to obtain the compound II-4 (5.1 mg, 11% yield). LC-MS, ES⁻ (m/z): 683.36 (M-1). ¹H NMR (400 MHz, CDCl₃) δ 7.87 (d, 2H), 7.37 (d, 2H), 3.71 (s, 1H), 3.09-2.96 (m, 5H), 1.38-1.31 (m, 9H), 1.16-1.05 (m, 25H), 0.97-0.80 (m, 8H), 0.65 (s, 1H).

### Example 90. Synthesis of Compound 12-1

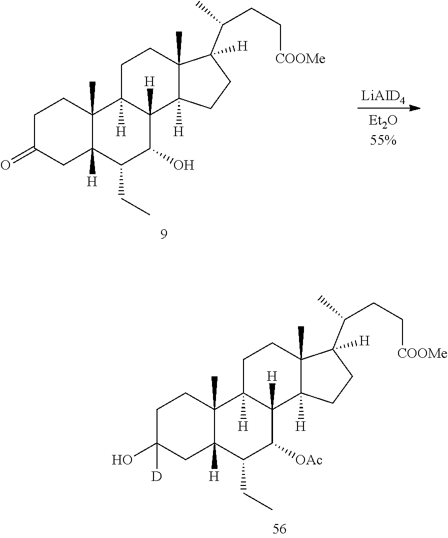

Step 1: Synthesis of Compound 56. To a solution of compound 9 (1 g) in Et₂O (10 mL) and then cooled to 0° C. was added LiAlD₄ (200 mg), and the mixture was stirred at room temperature for 3 h. After 10 mL water was added, and the mixture was extracted with EA (10 mL). The combined organic layers were dried over anhydrous Na₂SO₄, filtered, and concentrated in vacuo to give the desired product 56 (557 mg, 55%.).

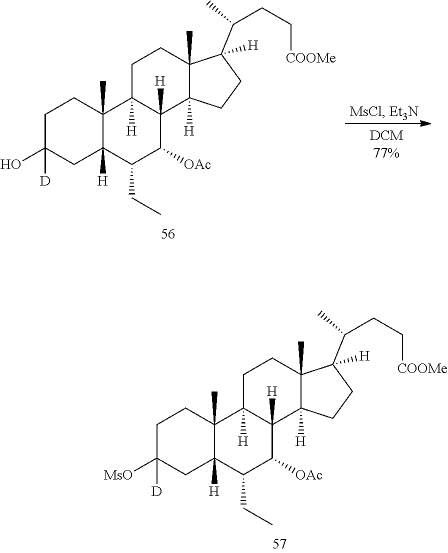

Step 2: Synthesis of Compound 57. To a solution of 56 (550 mg) in DCM (5 mL) was added Et₃N (0.4 mL) and then cooled to 0° C. was added MsCl (0.15 mL), and the mixture was stirred at RT for 2 h. After 10 mL water was added, and the mixture was extracted with EA (10 mL). The combined organic layers were dried over anhydrous Na₂SO₄, filtered, and concentrated in vacuo. The crude product was purified by silica gel chromatography to give the desired product 57 (480 mg, 75%). ¹H NMR (400 MHz, CDCl3) δ 4.41 (d, J=21.3 Hz, 1H), 3.61 (s, 3H), 3.16 (s, 3H), 2.20 (s, 3H), 2.00-0.79 (m, 41H).

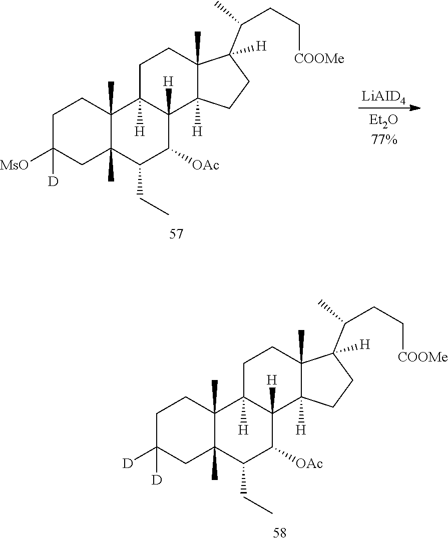

Step 3: Synthesis of Compound 58. To a solution of compound 57 (240 mg) in Et₂O (2 mL) and then cooled to 0° C. was added LiAlD₄ (40 mg), and the mixture was stirred at room temperature for 3 h. After 10 mL water was added, and the mixture was extracted with EA (10 mL). The combined organic layers were dried over anhydrous Na₂SO₄, filtered, and concentrated in vacuo to give the desired product 58 (154 mg, 77%).

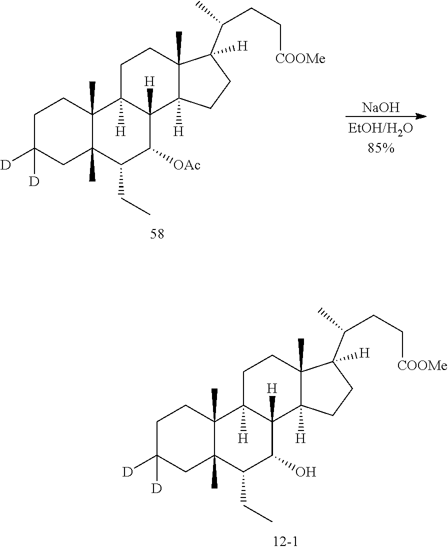

Step 4: Synthesis of Compound 12-1. To a solution of compound 58 (150 mg) in MeOH (3 mL) was added NaOH (4.5 g), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 12-1 (113 mg, 85%).

### Example 91. Synthesis of Compound 12-2

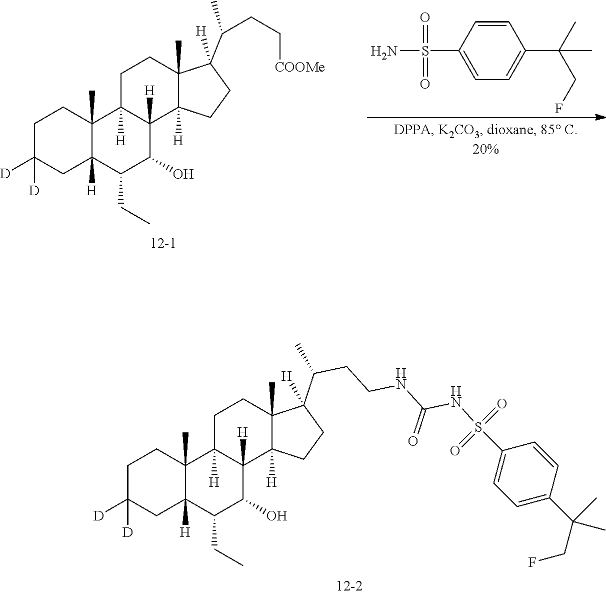

To a solution of compound 12-1 (110 mg) in 1,4-dioxane (10 mL) was added 4-(1-fluoro-2-methylpropan-2-yl)-benzene-sulfonamide (60 mg) and K₂CO₃ (108 mg), and the mixture was stirred at 85° C. for 3 h. After cooling to room temperature. Solvent was removed, and the residue was extracted with EA. The combined organic layers were dried over anhydrous Na₂SO₄, filtered, and concentrated in vacuo. The crude product was purified by silica gel chromatography to give the desired product 12-2 (30 mg, 20%). LC-MS, ES⁻ (m/z): 633.41 (M-1). ¹H NMR (400 MHz, Chloroform-d) δ 7.84 (dd, J=13.8, 8.4 Hz, 2H), 7.45-7.31 (m, 2H), 6.51 (s, 1H), 3.69 (d, J=2.8 Hz, 1H), 3.29 (s, 1H), 3.21-3.10 (m, 1H), 3.00 (d, J=2.4 Hz, 1H), 2.94 (d, J=2.3 Hz, 1H), 1.98-1.57 (m, 9H), 1.53-0.90 (m, 33H), 0.65 (s, 3H).

### Example 92. Synthesis of Compound 13-1

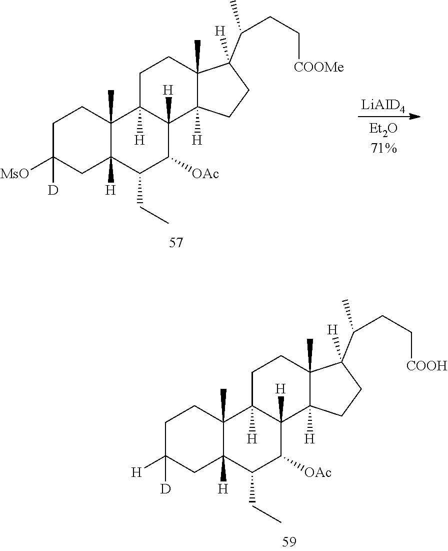

Step 1: Synthesis of Compound 59. To a solution of compound 57 (240 mg) in Et₂O (2 mL) and then cooled to 0° C. was added LiAlH₄ (40 mg), and the mixture was stirred at room temperature for 3 h. After 10 mL water was added, and the mixture was extracted with EA (10 mL). The combined organic layers were dried over anhydrous Na₂SO₄, filtered, and concentrated in vacuo to give the desired product 59 (143 mg, 71%.).

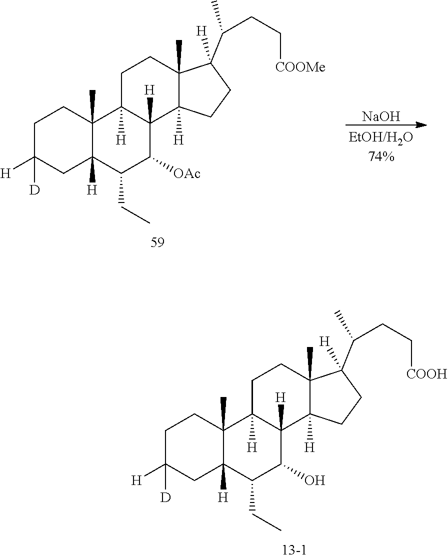

Step 2: synthesis o compound 13-1. To a solution o 59 140 mg in Me OH 3 mL was added NaOH (4.5 g), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 13-1 (118 mg, 74%).

### Example 93. Synthesis of Compound 13-2

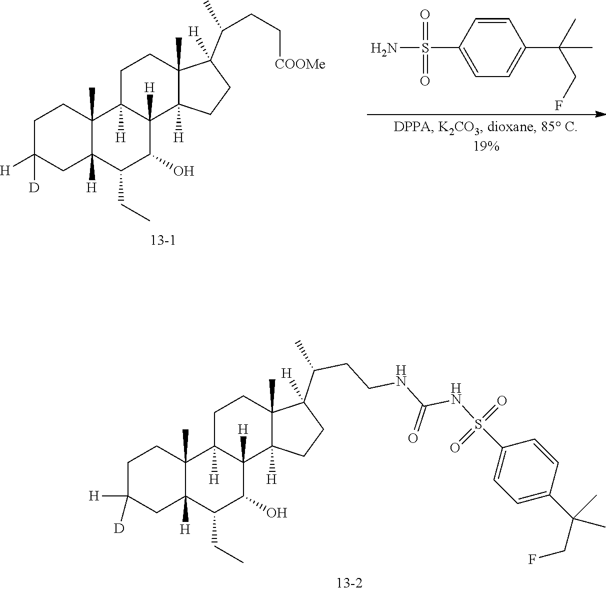

To a solution of 13-1 (110 mg) in 1,4-dioxane (10 mL) was added 4-(1-fluoro-2-methylpropan-2-yl)-benzene-sulfonamide (60 mg) and K₂CO₃ (108 mg), and the mixture was stirred at 85° C. for 3 h. After cooling to room temperature. Solvent was removed, and the residue was extracted with EA. The combined organic layers were dried over anhydrous Na₂SO₄, filtered, and concentrated in vacuo. The crude product was purified by silica gel chromatography to give the desired product 13-2 (28 mg, 19%). LC-MS, ES⁻ (m/z): 632.11 (M-1). ¹H NMR (400 MHz, Chloroform-d) δ 7.84 (dd, J=13.8, 8.4 Hz, 2H), 7.45-7.31 (m, 2H), 6.51 (s, 1H), 3.69 (d, J=2.8 Hz, 1H), 3.29 (s, 1H), 3.21-3.10 (m, 1H), 3.00 (d, J=2.4 Hz, 1H), 2.94 (d, J=2.3 Hz, 1H), 1.98-1.57 (m, 9H), 1.53-0.90 (m, 33H), 0.65 (s, 3H).

### Example 94. Synthesis of Compound 14-1

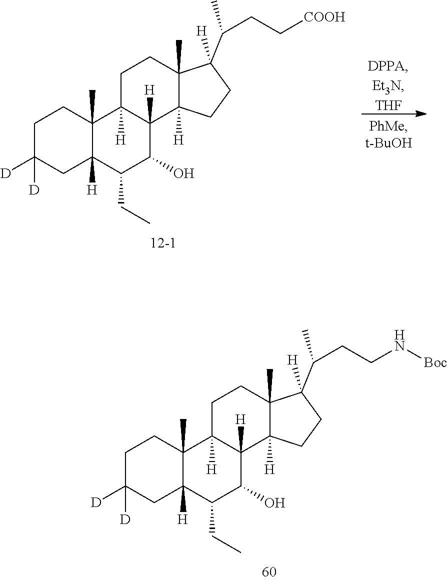

Step 1: Synthesis of Compound 60. Compound 12-1 (450 mg, 1.02 mmol) was dissolved in t-BuOH (10 mL). Crushed molecular sieves (4A) were added and the reaction mixture was stirred for 30 min under an argon atmosphere. Et₃N (206 mg, 2.04 mmol) and DPPA (337 mg, 1.22 mmol) were added and the reaction mixture was heated under reflux. After 4 h the solution was filtered and concentrated in vacuum, the crude product was purified by silica gel chromatography to give the 60 (255.0 mg, 67% yield).

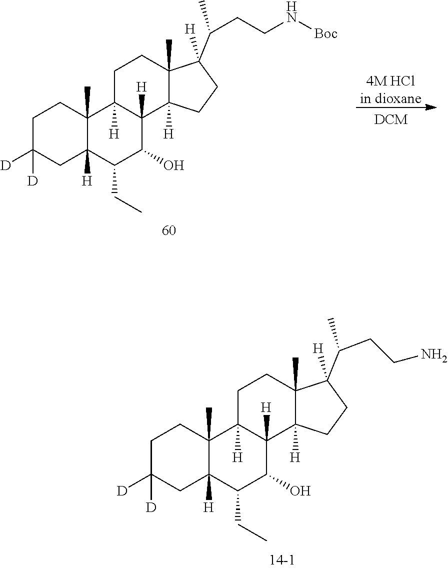

Step 2: synthesis of 14-1. Into a 40 mL vial were added 60 (224.0 mg, 0.44 mmol) and HCl-dioxane (5 mL) at room temperature. A mixture was stirred for 2 h at room temperature under air atmosphere. The reaction was monitored by TLC. The resulting mixture was concentrated under vacuum. This resulted in compound 14-1 (238 mg, 100% yield) as a white solid. LC-MS, ES⁻ (m/z): 410.33 (M-1).

¹H NMR (400 MHz, CDCl₃) δ 3.37-3.11 (m, 1H), 2.49-2.29 (m, 1H), 2.04-1.85 (m, 3H), 1.84-1.75 (m, 3H), 1.73-1.62 (m, 5H), 1.56-1.53 (m, 2H), 1.51-1.33 (m, 7H), 1.25-1.14 (m, 5H), 1.00-0.78 (m, 10H), 0.72-0.61 (m, 3H).

### Example 95. Synthesis of Compound 14-2

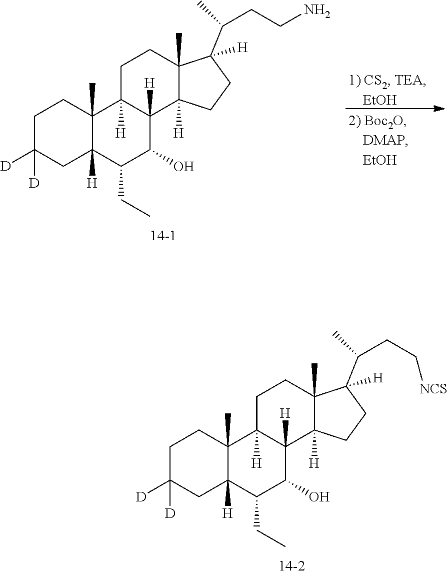

To a solution of 14-1 (238 mg, 0.58 mmol) in anhydrous ethanol was added CS₂ (441 mg, 5.8 mmol) and triethyl amine (586 mg, 5.8 mmol). The mixture was stirred at room temperature until intermediate was formed. Then, the reaction was cooled in an ice bath at −5° C. Di-tert-butyl di-carbonate (Boc₂O) (126 mg, 0.58 mmol) and DMAP (2 mg, 0.017 mmol) was dissolved in 0.5 mL anhydrous ethanol, separately. At first, di-tert-butyl di-carbonate solution was added to the reaction mixture, and then the addition of DMAP solution was performed immediately. The reaction was kept in an ice bath for 5 min and then was allowed to reach room temperature. The reaction was monitored by TLC. The reaction mixture was concentrated under vacuum and 10 mL water was added to residue. The mixture was extracted with chloroform (3×20 mL) and organic phases were collected, dried over anhydrous Na₂SO₄ and concentrated under vacuum. The products were purified by silica gel chromatography to give the 14-2 (80.0 mg, 30% yield). LC-MS, ES⁻ (m/z): 452.29 (M-1). ¹H NMR (400 MHz, CDCl₃) δ 3.72 (s, 1H), 3.64-3.40 (m, 2H), 2.56-2.30 (m, 1H), 2.06-1.68 (m, 9H), 1.51-1.39 (m, 5H), 1.38-1.27 (m, 6H), 1.22-1.11 (m, 4H), 1.05-0.80 (m, 10H), 0.72-0.65 (m, 3H).

### Example 96. Synthesis of Compound 14-3

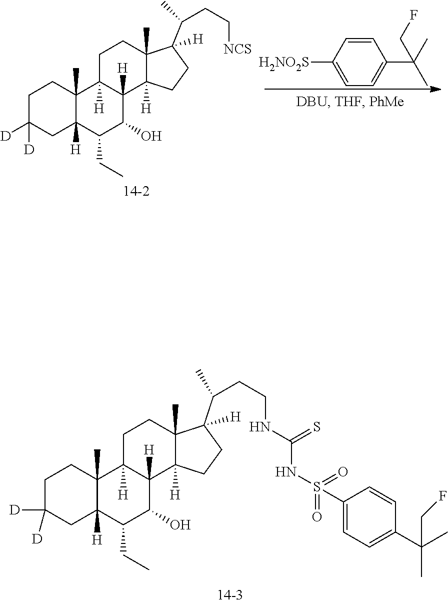

To a solution of 14-2 (80 mg) in 1,4-dioxane (10 mL) was added sulfonamide (56 mg) and K₂CO₃ (98 mg), and the mixture was stirred at 85° C. for 3 h. After cooling to room temperature. Solvent was removed, and the residue was extracted with EA. The combined organic layers were dried over anhydrous Na₂SO₄, filtered, and concentrated in vacuo. The crude product was purified by silica gel chromatography to give the desired product 14-3 (30 mg, 24%). LC-MS, ES⁻ (m/z): 663.41 (M-1). ¹H NMR (400 MHz, Chloroform-d) δ 7.84 (dd, J=13.8, 8.4 Hz, 2H), 7.45-7.31 (m, 2H), 6.51 (s, 1H), 3.69 (d, J=2.8 Hz, 1H), 3.29 (s, 1H), 3.21-3.10 (m, 1H), 3.00 (d, J=2.4 Hz, 1H), 2.94 (d, J=2.3 Hz, 1H), 1.98-1.57 (m, 9H), 1.53-0.90 (m, 33H), 0.65 (s, 3H).

### Example 97. Synthesis of Compound 14-4

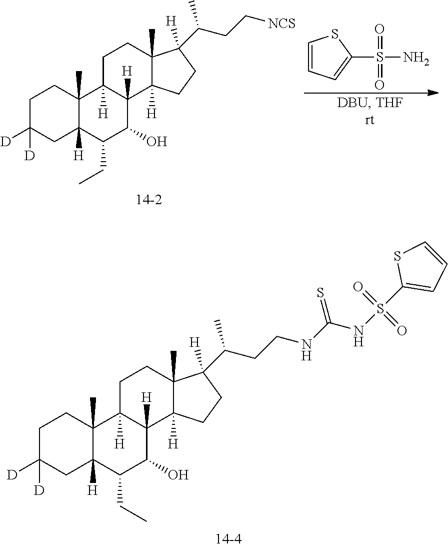

The sulfonamide (10 mg) and DBU (46 mg, 0.3 mmol) in THF (1 ml) was added into a solution of the 14-2 (41.7 mg, 0.1 mmol) in toluene (1 ml). The mixture was stirred at RT overnight. The mixture was quenched with water, extracted with EA, dried, filtered, and concentrated. The crude product was purified by silica gel chromatography to give the desired product 14-4 (8 mg). LC-MS, ES⁻ (m/z): 582.30 (M-1). ¹H NMR (400 MHz, Chloroform-d) δ 8.08 (s, 1H), 7.73-7.69 (m, 2H), 7.15 (dd, J=4.9, 4.0 Hz, 1H), 3.72-3.47 (m, 3H), 2.02-1.07 (m, 27H), 0.99 (d, J=6.5 Hz, 3H), 0.93-0.87 (m, 6H), 0.67 (s, 3H).

### Example 98. Synthesis of Compound 15-1

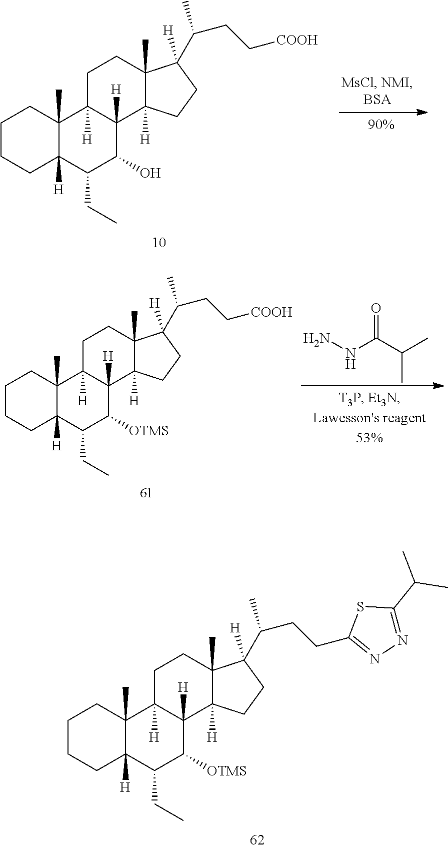

Step 1: Synthesis of Compound 61. Compound 10 (1.06 g, 2.62 mmol) was dissolved in DCM (20 mL), and then which was cooled to 0° C. To the above solution, was added N-methyl imidazole (0.83 mL, 10.47 mmol), bis(trimethylsilyl)acetamide (3.2 mL, 13.09 mmol) and MsCl (1.0 mL, 7.85 mmol) slowly. The reaction was allowed to stir at room temperature for 2.5 hours before concentration. The residue was then redissolved in EtOAc and washed with 10% aq. citric acid solution. The organic layer was dried by Na₂SO₄, filtered and concentrated. The crude residue was then purified by Flash chromatography (30 g SiO₂, EtOAc/hexanes=0 to 50%) to give the compound 61 as a pale white solid, 1.14 g, 90% yield. LC-MS, ES⁻ (m/z): 475.37 (M-1).
Step 2: Synthesis of Compound 62. To a solution of compound 61 (51.17 mg, 0.106 mmol) in EtOAc (2 mL) was added isobutyro-hydrazide (12.88 mg, 0.111 mmol), triethyl amine (37.1 μL, 0.265 mmol), Lawesson's reagent (64.4 mg, 0.159 mmol) and propyl-phosphonic anhydride (98 μL 50 wt % EtOAc solution, 0.227 mmol) at room temperature. The reaction was allowed to stir at 80° C. for 7 hours before quenched by aq. NaHCO₃ at room temperature. The mixture was further extracted by EtOAc (25 mL×3). The combined organic layers were dried over Na₂SO₄, filtered and concentrated. The crude was then purified by Flash chromatography (9 g SiO₂, EtOAc/hexanes=0 to 30%) to give the compound 62 as a pale white solid, 31.7 mg, 53% yield. LC-MS, ES⁺ (m/z): 558.41 (M+1).

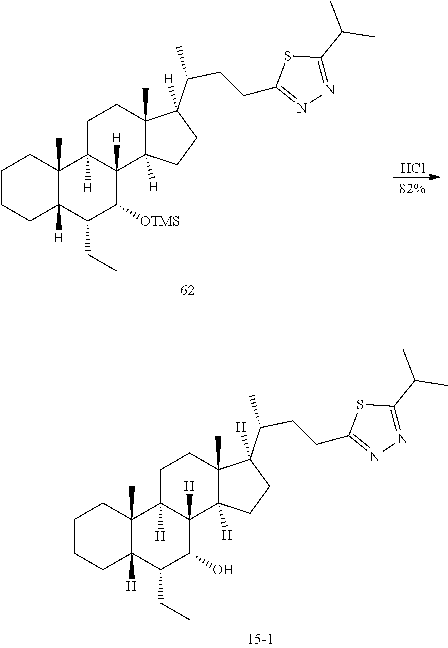

Step 3: Synthesis of Compound 15-1. Compound 62 (31.7 mg, 0.056 mmol) was dissolved in MeOH/THF (1 mL/0.5 mL). 1 drop of aq. 37% HCl was added and the reaction mixture was stirred at room temperature for 10 min before concentration. The crude was purified by Flash chromatography (4 g SiO₂, EtOAc/hexanes=0 to 100%) to give the product of 15-1 as a white solid, 22.4 mg, 82% yield. LC-MS, ES⁺ (m/z): 487.36 (M+1).

### Example 99. Synthesis of Compound 15-2

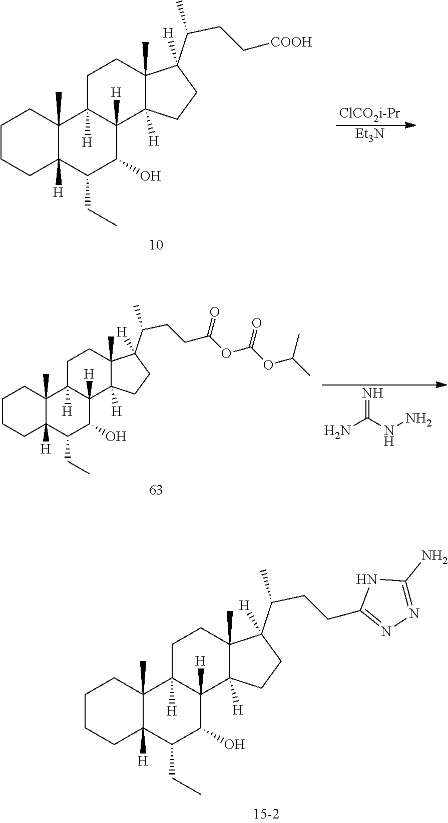

Step 1: Synthesis of Compound 63. To a solution of compound 10 (564.5 mg, 1.395 mmol) in THF (5 mL) was added triethyl amine (0.78 mL, 5.6 mmol) and isopropyl chloroformate (3.07 mL 1.0 M PhMe solution, 3.07 mmol) slowly at 0° C. The reaction was allowed to warm to room temperature and stir for 1 hour. The solvent was removed under reduced pressure. The crude residue was redissolved in EtOAc and washed with water. The aq. layer was then extracted with ethyl acetate (15 mL*2). The combined organic layers were dried over Na₂SO₄, filtered and concentrated under reduced pressure. The crude oil of compound 63 was directly used in the following step without any further purification. LC-MS, ES⁺ (m/z): 491.37 (M+1).
Step 2: Synthesis of Compound 15-2. To a solution of crude compound 63 obtained from previous step in toluene (6 mL) was added hydrazinecarboximidamide bicarbonate (128.2 mg, 1.09 mmol). The reaction was allowed to stir at 110° C. for 20 hours. The crude was filtered and purified by Flash chromatography (20 g SiO₂, MeOH/DCM=0 to 10%) to give the amino-triazole compound 15-2 as a white solid, 197.6 mg, 32% yield over steps. LC-MS, ES⁺ (m/z): 443.77 (M+1).

### Example 100. Synthesis of Compound 15-3

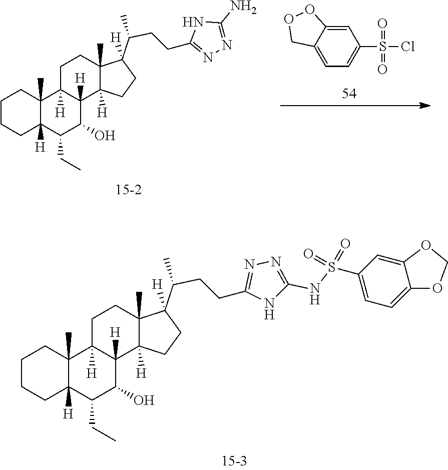

To a solution of compound 15-2 (18.95 mg, 0.0428 mmol) in DCM (1 mL) was added sulfonyl chloride 54 (4 μL, 0.0513 mmol) and triethyl amine (18 μL, 0.1284 mmol) at 0° C. The reaction was allowed to stir at room temperature for overnight. The solvent was removed under reduced pressure. The crude residue was purified by Flash chromatography (4 g SiO₂, MeOH/DCM=0 to 20%) to give the sulfonamide compound 15-3 as a white solid, 16.1 mg, 60% yield. LC-MS, ES⁺ (m/z): 626.77 (M+1).

### Example 101. Synthesis of Compound 16-1

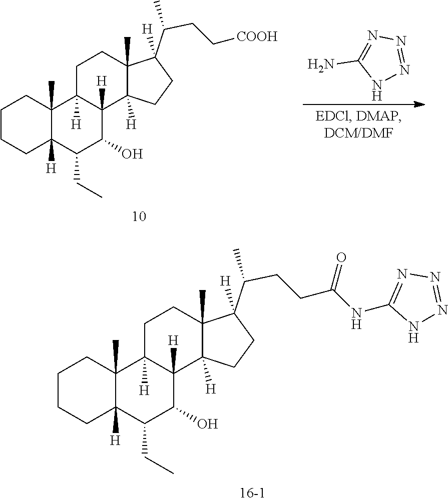

To a solution of compound 10 (40.5 mg, 0.1 mmol) in 2 mL of DMF/CH₂Cl₂ solution (1:1, v/v) was charged 5-Amino-1,2,3,4-tetrazole (25.5 mg, 0.299 mmol), EDCI (38.3 mg, 0.2 mmol) and DMAP (24.4 mg, 0.2 mmol). The resulting solution was allowed to stir at 60° C. until completion indicated by TLC. The solvent was removed under reduced pressure. Water (15 mL) and 1 N HCl aq. solution (2 mL) was added. The solution was then extracted with ethyl acetate (20 mL*3). The combined organic layer was dried over Na₂SO₄, filtered and concentrated under reduced pressure. The residue was purified by Flash chromatography (10 g SiO₂, MeOH/DCM=0 to 10%) to give the compound 16-1 as a pale white solid, 24.1 mg, 51% yield. LC-MS, ES⁺ (m/z): 472.36 (M+1).

### Example 102. Synthesis of Compound 16-2

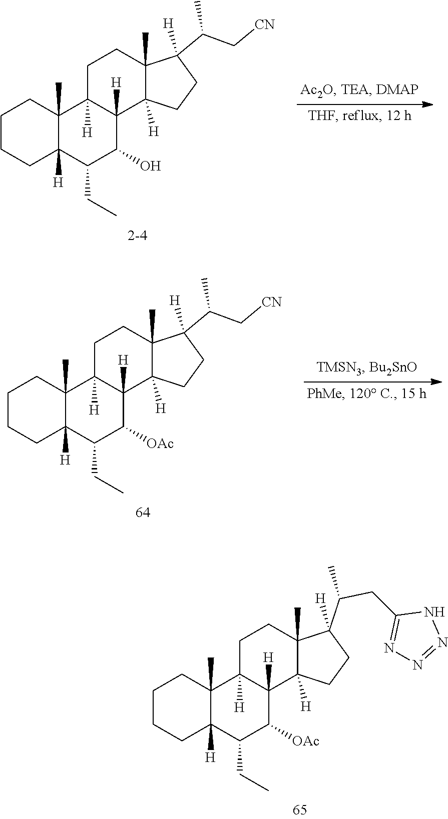

Step 1: Synthesis of Compound 64. To a solution of 2-4 (148 mg, 0.4 mmol, 1.0 eq) in THF (2 mL), were added TEA (81 mg, 8.0 mmol, 20.0 eq), 4-dimethylaminopyridine (6.3 mg, 0.04 mmol, 0.1 eq), and acetic anhydride (87.3 mg, 8.6 mmol, 20.0 eq). The resulting solution was stirred at 90° C. for 12 hours. After being cooled to rt, it was concentrated and the residue was dissolved in ethyl acetate (2 mL), then was washed with water (5 mL*2), saturated NaCl (5 mL*2). The organic layer was dried over Na₂SO₄, filtered and concentrated. The residue was purified by flash chromatography (EtOAc/hexanes=0 to 20%) to give the desired compound 64 as a yellow solid, 155.5 mg, 94% yield. LC-MS, ES⁺ (m/z): 414.73 (M+1).
Step 2: Synthesis of Compound 65. To a solution of compound 64 (132 mg, 0.32 mmol) in toluene (6 mL) was added hydrazinecarboximidamide bicarbonate (64.1 mg, 0.545 mmol), azidotrimethylsilane (1.85 g, 16 mmol), dibutylstannanone (0.75 g, 3.2 mmol) in toluene (5 mL). The resulting solution was stirred at 120° C. for 15 hours. After cooling to rt the solution was diluted with 50 mL of water and extracted with ethyl acetate (25 mL*3). The combined organic layer was concentrated under vacuum to give the desired compound 65 as a yellow oil, which was used for next step without further purification. LC-MS, ES⁺ (m/z): 457.77 (M+1).

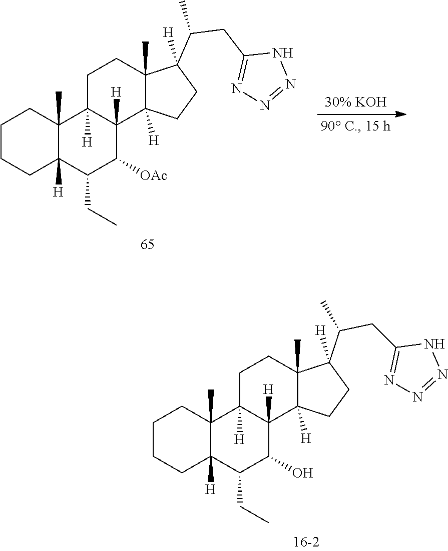

Step 3: Synthesis of Compound 16-2. Into a 25-mL round-bottom flask, was placed a solution of compound 65 (0.32 mmol), methanol (5 mL) and 30% potassium hydroxide (5 mL). The resulting solution was stirred at 90° C. for 15 hours. After cooling to rt the pH value of the solution was adjusted to 6 with con HCl and extracted with ethyl acetate (15 mL*3). The combined organic layer was concentrated and purified by flash chromatography (EtOAc/hexanes=0 to 40%) to give the desired compound 16-2 as a pale white solid, 26.5 mg, 20% yield. LC-MS, ES⁺ (m/z): 415.33 (M+1).

### Example 103. Synthesis of Compound 17-1

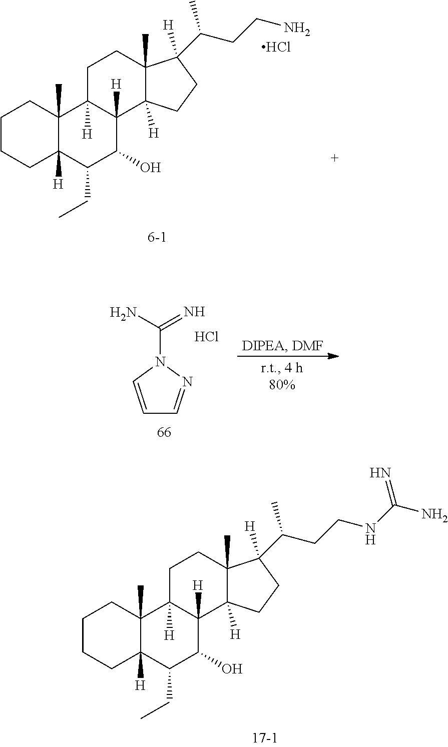

1H-pyrazole-1-carboxamidine hydrochloride 66 (298 mg, 2.03 mmol, 1.1 eq) was suspended in 10 mL of dimethylformamide under nitrogen. Diisopropylethylamine (DIPEA) (0.354 mL, 0.263 g, 2.03 mmol, 1.1 eq) was added, yielding a clear orange solution. A solution of compound 6-1 (762 g, 1.85 mmol, 1.0 eq) in 1 mL of DMF was added dropwise, immediately forming a precipitate. The mixture was stirred at room temperature under nitrogen for 4 hours until it became a clear solution again. The solvents were removed in vacuo and the remained viscous orange oil was treated with ˜10 mL saturated NaHCO₃ solution, immediately forming a white crystalline precipitate, which was filtered off, washed with NaHCO₃ solution and diethyl ether, and dried in vacuo yielding the title product 17-1 as an off-white crystalline solid (618 mg, 80% yield). LC-MS, ES⁺ (m/z): 418.38 (M+1), 835.78 (6%, [2M+1]). Calculated for [C₂₆H₄,N₃O+H]⁺: 417.68.

### Example 104. Synthesis of Compound 17-2

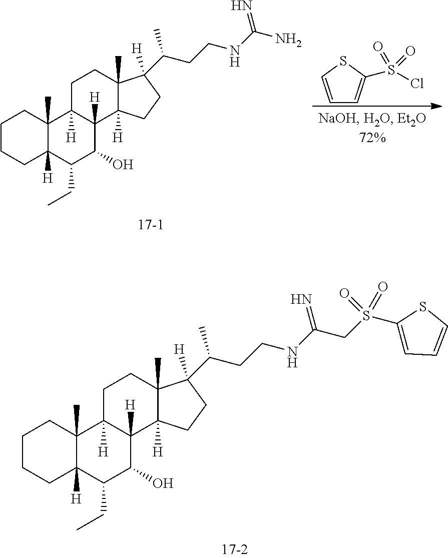

A solution of 2-thiophenesulfonyl chloride (21 mg, 0.115 mmol) in ethyl ether (1 ml) was added to a stirred solution of N-substituted-guanidine 17-1 (48 mg, 0.115 mmol) in 1N sodium hydroxide (1 ml), and the mixture was stirred at room temperature for 1 h. The resulting precipitate was isolated by filtration, washed with ethyl ether and dried to give the title compound 17-2 (46.7 mg, 72% yield) as a white solid. LC-MS, ES⁺ (m/z): 564.74 (M+1).

### Example 105. Synthesis of Compound 18-1

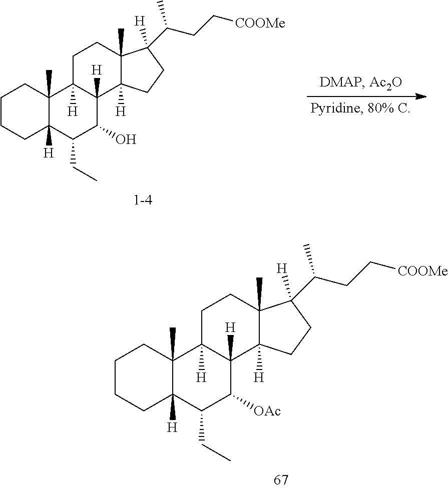

Step 1: Synthesis of Compound 67. Compound 1-4 (2.0 g, 4.78 mmol), DMAP (58 mg, 0.478 mmol) and acetic anhydride (2.9 g, 28.68 mmol) were dissolved in pyridine (9 ml), heated at 80° C. and stirred overnight. Add 0.5 M HCl, then extract three times with EA, combine the organic phase, wash three times with 1M HCl, dry with anhydrous Na₂SO₄, evaporate the solvent. The crude product was purified by silica gel chromatography to give the desired product 67 (1.6 g, 74% yield). LC-MS, ES⁺ (m/z): 461.43 (M+1).

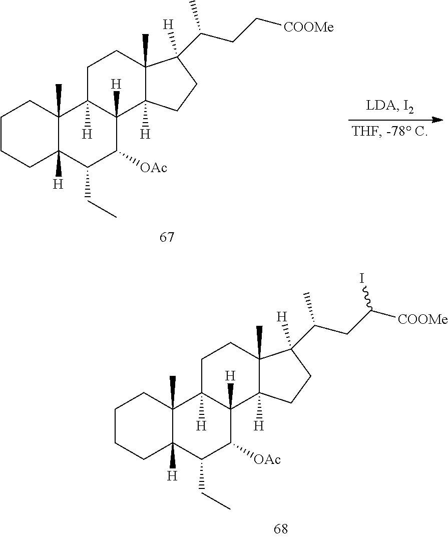

Step 2: Synthesis of Compound 68. To a solution of 67 (1.0 g, 2.17 mmol) in anhydrous THF (10 mL) at −78° C. was charged LDA (6.5 mL of a 1M solution in hexanes, 6.51 mmol) dropwise in argon atmosphere. The reaction was stirred for 15 min at −78° C. and a solution of I₂ (771 m g, 3.0 mmol) in anhydrous THY (4 mL) was added dropwise. The reaction was stirred for 0.5 h at −78° C. The reaction was poured into an aqueous 10% NH₄Cl solution (6 mL) and diluted with EA (10 mL). The layers were separated and the aqueous layer was extracted with EA (2*10 mL), dry with anhydrous Na₂SO₄, evaporate the solvent. The crude product was purified by silica gel chromatography to give the desired product 68 (547 mg, 43% yield). LC-MS, ES⁺ (m/z): 587.84 (M+1).

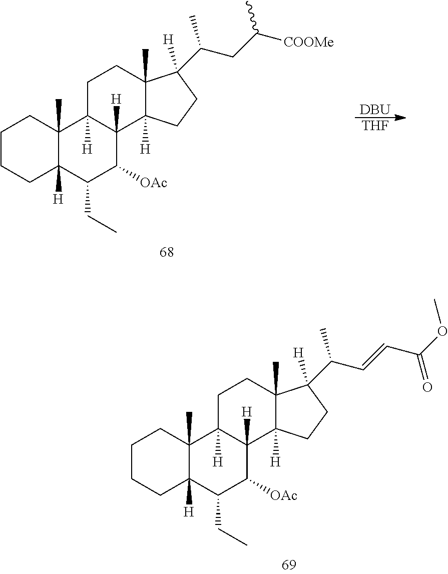

Step 3: Synthesis of Compound 69. To a solution of crude compound 68 (540 mg, 0.921 mmol) in anhydrous THF (10 mL) was charged DBU (420 mg, 2.76 mmol, 3.0 eq) and the reaction was stirred overnight. The reaction was quenched with aqueous 10% NH₄Cl (5 mL) and diluted with EA (10 mL). The layers were separated and the aqueous layer was extracted with EA (2*10 mL), dry with anhydrous Na₂SO₄, evaporate the solvent. The crude product was purified by silica gel chromatography to give the desired product 69 (388 mg, 92% yield). LC-MS, ES⁺ (m/z): 459.24 (M+1).

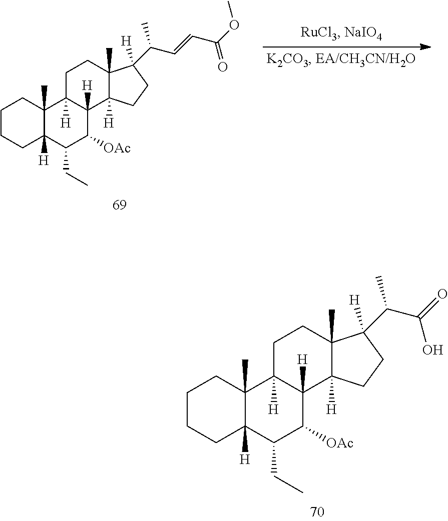

Step 4: Synthesis of Compound 70. To a solution of compound 69 (380 mg, 0.83 mmol) in EA (2 mL) and CH₃CN (2 mL) was added a solution of K₂CO₃ (1.2 g, 8.3 mmol) in H₂O (3 mL). RuCl₃ hydrate (17 mg, 0.083 mmol) was added, followed by NaIO₄ (1.77 g, 8.3 mmol) and the reaction was stirred 4 h. The reaction was quenched with aqueous 10% citric acid (5 mL) and diluted with EA (10 mL). The layers were separated and the aqueous layer was extracted with EA (2*10 mL), dry with anhydrous Na₂SO₄, evaporate the solvent. The crude product was purified by silica gel chromatography to give the desired product 70 (264 mg, 76% yield). LC-MS, ES⁻ (m/z): 417.46 (M-1).

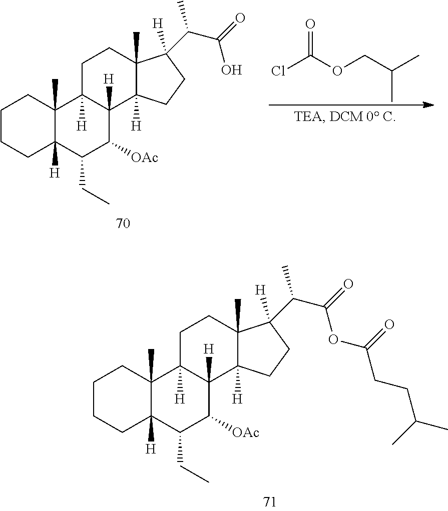

Step 5: Synthesis of Compound 71. Isobutyl chloroformate (102 mg, 0.75 mmol) was added dropwise to a solution of compound 70 (260 mg, 0.62 mmol) and Et₃N (94 mg, 0.93 mmol) in DCM (4 mL) at 0° C. The reaction was stirred at 0° C. for 30 min. The reaction was quenched with H₂O (3 mL) and diluted with DCM (5 mL). The layers were separated and the aqueous layer was extracted with DCM (2*5 mL), dry with anhydrous Na₂SO₄, evaporate the solvent. The crude product was purified by silica gel chromatography to give the desired product 71 (306 mg, 95% yield). LC-MS, ES⁺ (m/z): 519.34 (M+1).

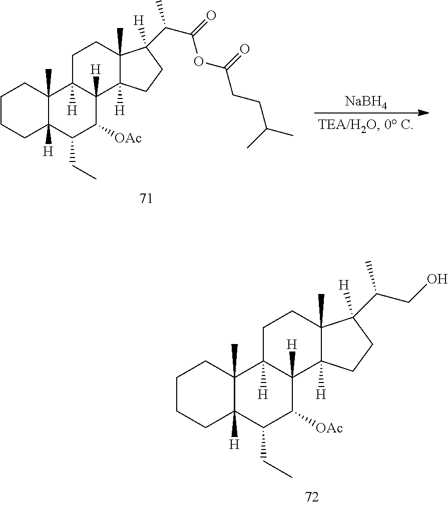

Step 6: Synthesis of Compound 72. NaBH₄ (22 mg, 0.579 mmol) was added to a solution of compound 71 (300 mg, 0.579 mmol) in THF (3 ml)/H₂O (0.7 ml) at 0° C. The reaction was stirred at 0° C. for 0.5 h and a second portion of NaBH₄ (22 mg, 0.579 mmol) was added. The reaction was stirred overnight. The reaction was cooled to 0° C., diluted with EA (3 mL) and quenched with 10% citric acid (3 mL). The layers were separated and the aqueous layer was extracted with EA (2*5 mL), dry with anhydrous Na₂SO₄, evaporate the solvent. The crude product was purified by silica gel chromatography to give the desired product 72 (208 mg, 89% yield). LC-MS, ES⁺ (m/z): 405.42 (M+1).

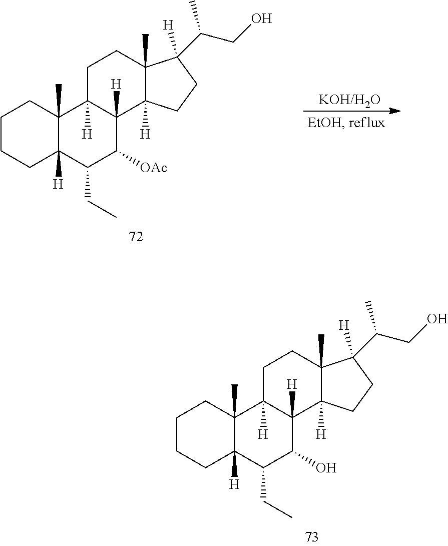

Step 7: Synthesis of Compound 73. To a solution of 72 (205 mg, 0.507 mmol) in EtOH (2 mL) was added KOH (284 mg, 5.07 mmol) in water (2 mL), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 73 (154 mg, 83% yield). LC-MS, ES⁺ (m/z): 363.16 (M+1).

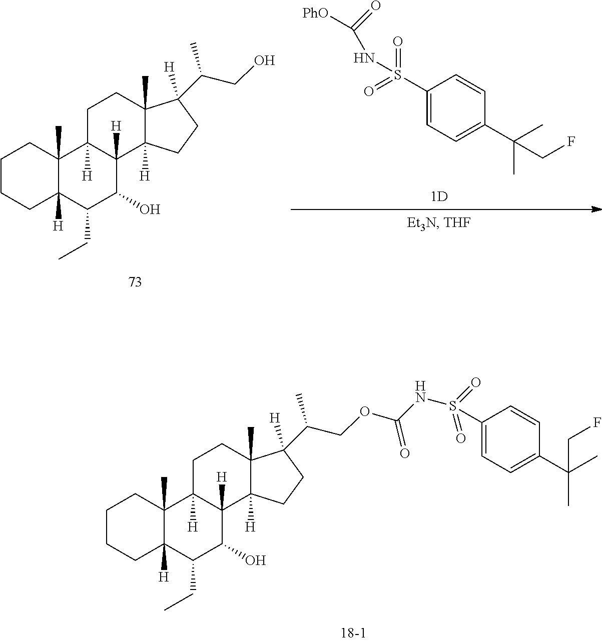

Step 8: Synthesis of Compound 18-1. Compound 1D (53 mg, 0.152 mmol) was added to a solution of compound 73 (50 mg, 0.138 mmol) and TEA (42 mg, 0.414 mmol) in dry THF (2 mL) at rt. The reaction mixture was stirred at rt for 4 h. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 18-1 (36 mg, 43% yield). LC-MS, ES⁺ (m/z): 620.26 (M+1).

### Example 106. Synthesis of Compound 18-2

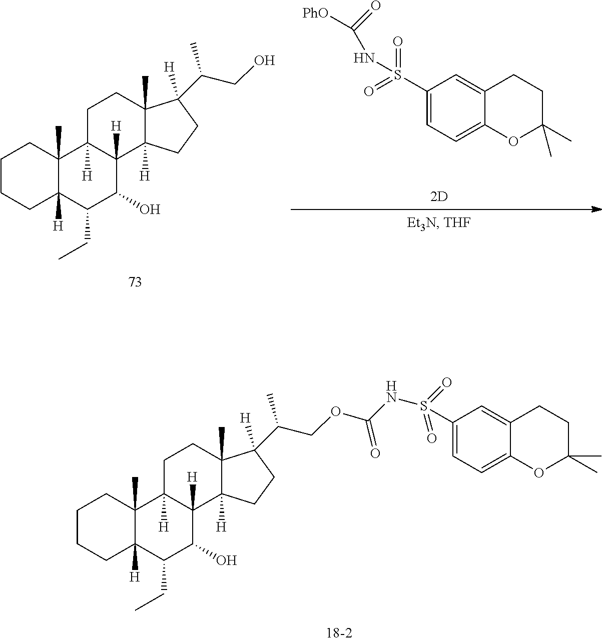

Compound 2D (55 mg, 0.152 mmol) was added to a solution of compound 73 (50 mg, 0.138 mmol) and TEA (42 mg, 0.414 mmol) in dry THF (2 mL) at rt. The reaction mixture was stirred at rt for 4 h. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 18-2 (31 mg, 36% yield). LC-MS, ES⁺ (m/z): 630.18 (M+1). 1H NMR (400 MHz, Chloroform-d) δ 7.76 (d, J=2.4 Hz, 1H), 7.74-7.70 (m, 1H), 7.40 (s, 1H), 6.87 (d, J=8.7 Hz, 1H), 4.14-4.08 (m, 1H), 3.79 (dd, J=10.5, 7.4 Hz, 1H), 3.68 (s, 1H), 2.83 (t, J=6.5 Hz, 2H), 1.95-1.10 (m, 33H), 0.94 (d, J=6.6 Hz, 3H), 0.91-0.87 (m, 6H), 0.64 (s, 3H).

### Example 107. Synthesis of Compound 18-3

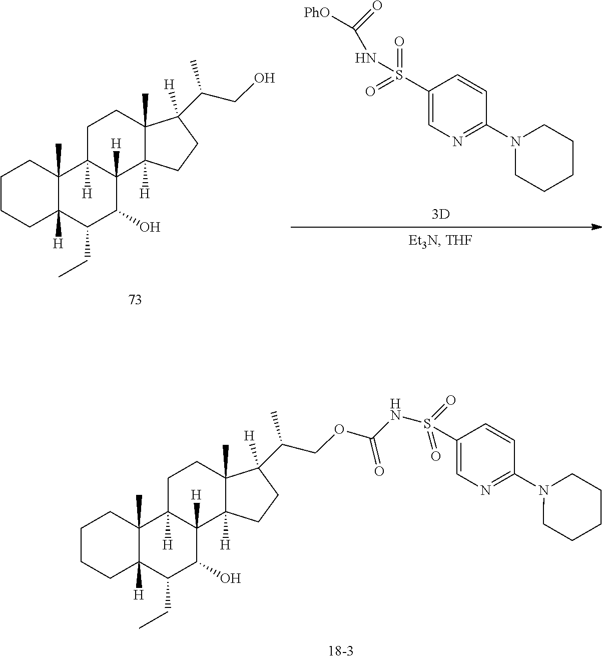

Compound 3D (55 mg, 0.152 mmol) was added to a solution of compound 73 (50 mg, 0.138 mmol) and TEA (42 mg, 0.414 mmol) in dry THF (2 mL) at rt. The reaction mixture was stirred at rt for 4 h. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 18-3 (33 mg, 39% yield). LC-MS, ES⁺ (m/z): 630.36 (M+1). 1H NMR (400 MHz, Chloroform-d) δ 8.66 (dd, J=2.6, 0.6 Hz, 1H), 7.92 (dd, J=9.3, 2.6 Hz, 1H), 7.59 (s, 1H), 6.59 (dd, J=9.4, 0.7 Hz, 1H), 4.15-4.07 (m, 2H), 3.79 (dd, J=10.4, 7.4 Hz, 1H), 3.72-3.66 (m, 4H), 1.94-1.11 (m, 31H), 0.96 (d, J=6.6 Hz, 3H), 0.92-0.86 (m, 6H), 0.65 (s, 3H).

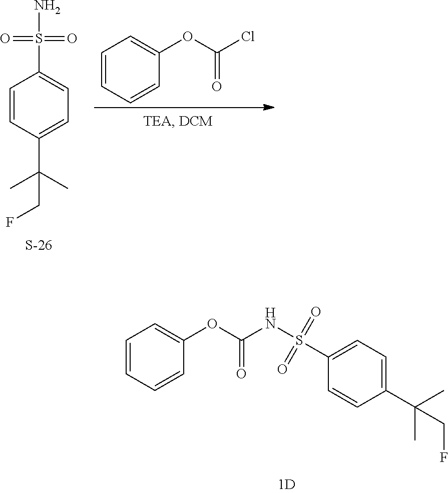

Synthesis of intermediate 1D. Phenyl chloroformate (16 mg, 0.104 mmol) was added dropwise to a suspension of s-26 (20 mg, 0.087 mmol) and TEA (26 mg, 0.26 mmol) in DCM (1 ml) at 0° C. The reaction mixture was stirred at 0° C. for 2 h. The reaction was quenched with H₂O (2 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 1D (26 mg, 85% yield). LC-MS, ES⁺ (m/z): 352.16 (M+1).

### Synthesis of Compound 2D

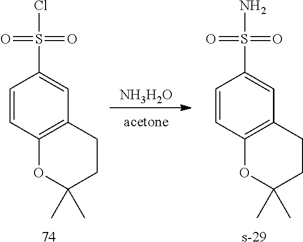

Step 1: Synthesis of compound s-29. A solution of compound 74 (2,2-dimethylchromane-6-sulfonyl chloride) (500 mg, 1.92 mmol) in acetone (2.5 ml) was added dropwise to NH4OH (aq) (28-30%, 2.5 ml) at 0° C. The reaction was stirred at rt for 2 h. The reaction was quenched with H₂O (2 mL) and diluted with EA (5 mL). The layers were separated and the aqueous layer was extracted with EA (2*5 mL), dry with anhydrous Na₂SO₄, evaporate the solvent. The crude product was purified by silica gel chromatography to give the desired product s-29 (403 mg, 87% yield). LC-MS, ES⁺ (m/z): 242.17 (M+1).

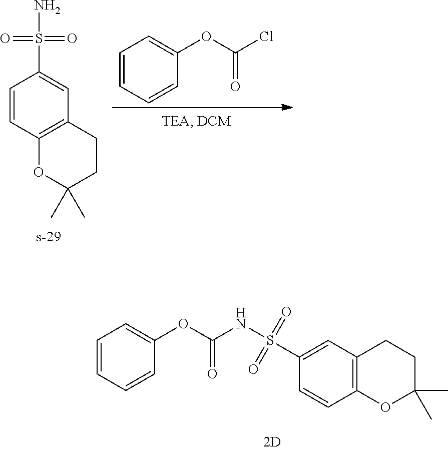

Step 2: Synthesis of intermediate 2D. Phenyl chloroformate (313 mg, 2.00 mmol) was added dropwise to a suspension of s-29 (403 mg, 1.67 mmol) and TEA (506 mg, 5.01 mmol) in DCM (5 ml) at 0° C. The reaction mixture was stirred at 0° C. for 2 h. The reaction was quenched with H₂O (3 mL) and diluted with DCM (5 mL). The layers were separated and the aqueous layer was extracted with DCM (2*5 mL), dry with anhydrous Na₂SO₄, evaporate the solvent. The crude product was purified by silica gel chromatography to give the desired product 2D (561 mg, 93% yield). LC-MS, ES⁺ (m/z): 362.27 (M+1).

### Synthesis of Compound 2D

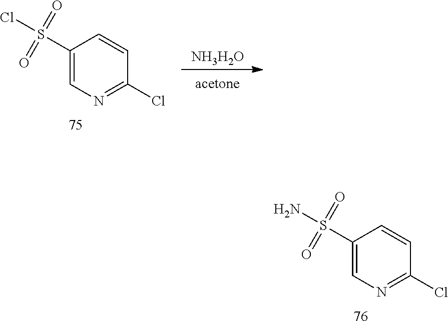

Step 1: Synthesis of compound 76. A solution of compound 75 (6-chloropyridine-3-sulfonyl chloride) (1.0 g, 4.72 mmol) in acetone (5 ml) was added dropwise to NH₃H₂O (aq) (28-30%, 5 ml) at 0° C. The reaction was stirred at rt for 2 h. The reaction was quenched with H₂O (3 mL) and diluted with EA (5 mL). The layers were separated and the aqueous layer was extracted with EA (2*10 mL), dry with anhydrous Na₂SO₄, evaporate the solvent. The crude product was purified by silica gel chromatography to give the desired product 76 (846 mg, 93% yield). LC-MS, ES⁺ (m/z): 193.12 (M+1).

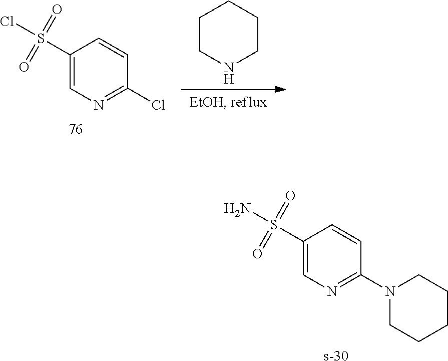

Step 2: Synthesis of compound s-30. A solution of compound 76 (846 mg, 4.39 mmol) in EtOH (5 ml) was added piperidine (449 mg, 5.27 mmol) at rt. The reaction was stirred at 85° C. overnight, cooled down to rt, and the precipitated solids (after 3 h aging) were collected by filtration and rinsed with EtOH, and dried. The solids were further purified by mixing with water, filtration, and washing with water, and then dried to give compound s-30 as a white solid (963 mg, 91% yield). LC-MS, ES⁺ (m/z): 242.21 (M+1).

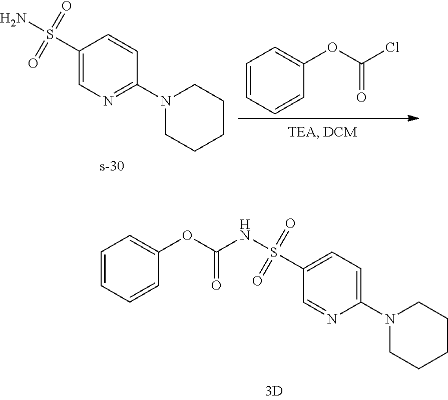

Step 2: Synthesis of intermediate 3D. Phenyl chloroformate (750 mg, 4.79 mmol) was added dropwise to a suspension of s-30 (963 mg, 3.99 mmol) and TEA (1.2 g, 11.98 mmol) in DCM (10 ml) at 0° C. The reaction mixture was stirred at 0° C. for 2 h. The reaction was quenched with H₂O (5 mL) and diluted with DCM (5 mL). The layers were separated and the aqueous layer was extracted with DCM (2*10 mL), dry with anhydrous Na₂SO₄, evaporate the solvent. The crude product was purified by silica gel chromatography to give the desired product 3D (1.28 g, 89% yield). LC-MS, ES⁺ (m/z): 362.25 (M+1).

### Example 108. Synthesis of Compound 19-1

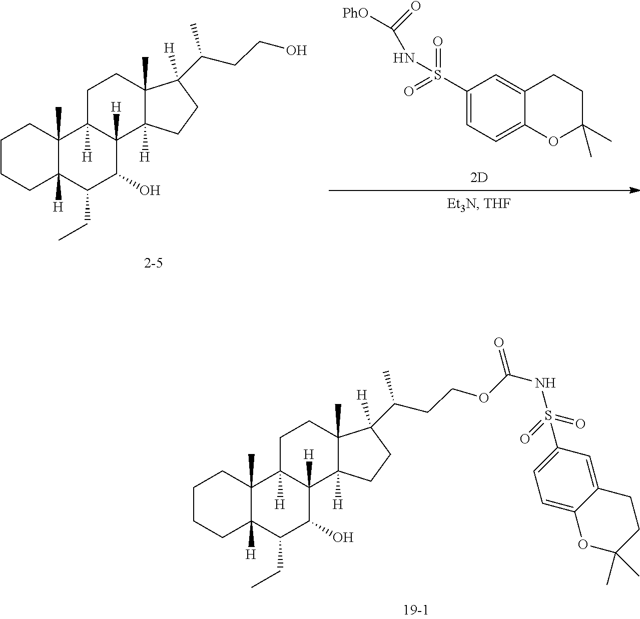

Compound 2D (53 mg, 0.146 mmol) was added to a solution of compound 2-5 (50 mg, 0.133 mmol) and TEA (40 mg, 0.4 mmol) in dry THF (2 mL) at rt. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 19-1 (25 mg, 29% yield). LC-MS, ES⁺ (m/z): 644.28 (M+1).

### Example 109. Synthesis of Compound 19-2

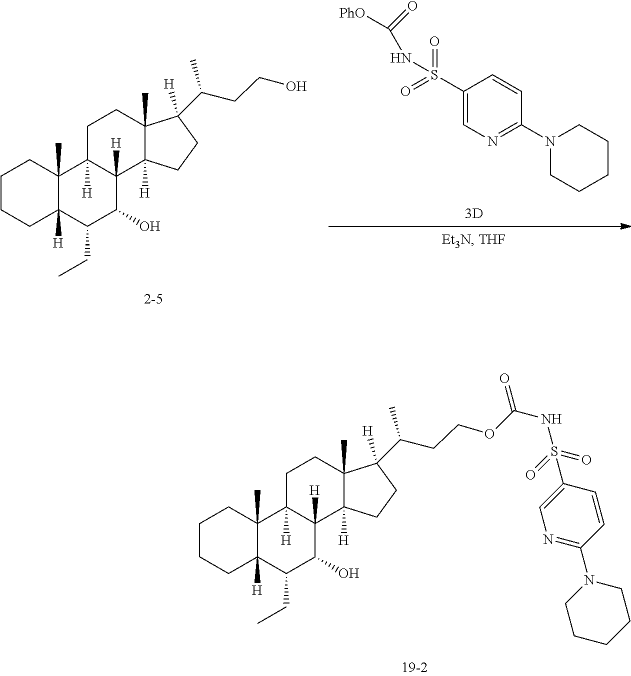

Compound 3D (53 mg, 0.146 mmol) was added to a solution of compound 2-5 (50 mg, 0.133 mmol) and TEA (40 mg, 0.4 mmol) in dry THF (2 mL) at rt. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 19-2 (28 mg, 32% yield). LC-MS, ES⁺ (m/z): 644.16 (M+1).

### Example 110. Synthesis of Compound 19-3

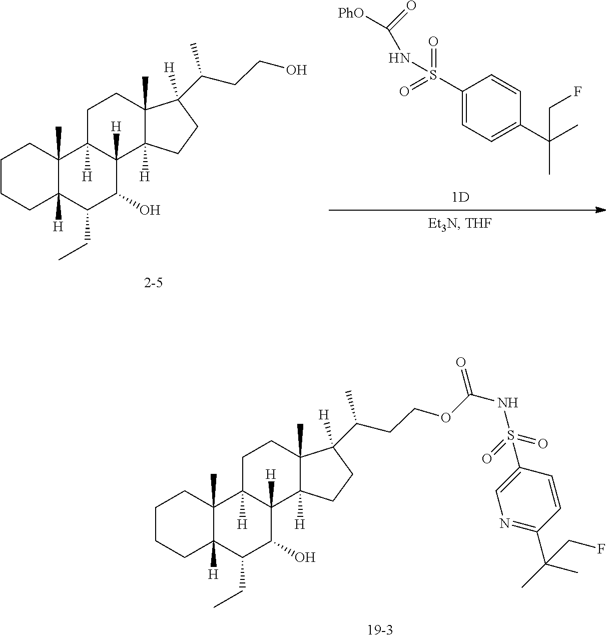

Compound ID (51 mg, 0.146 mmol) was added to a solution of compound 2-5 (50 mg, 0.133 mmol) and TEA (40 mg, 0.4 mmol) in dry THF (2 mL) at rt. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 19-3 (18 mg, 22% yield). LC-MS, ES⁺ (m/z): 634.47 (M+1).

### Example 111. Synthesis of Compound 20-1

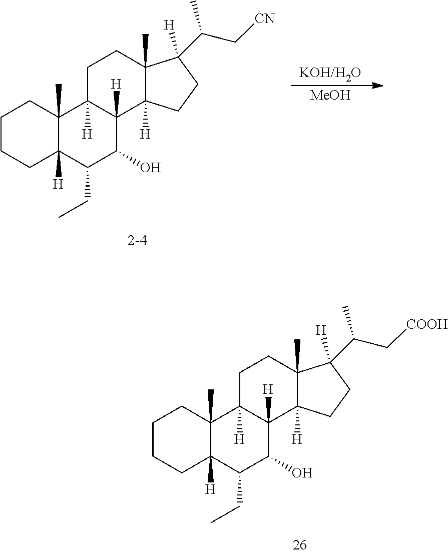

Step 1: alternative method for the synthesis of compound 26. A solution of 2-4 (200 mg, 0.538 mmol) in MeOH (2 ml) was added KOH (301 mg, 5.38 mmol) and H₂O (2 ml). The mixture was stirred at 90° C. for 16 h. The reaction mixture was quenched with 6N HCl to adjust pH to 5-6, extracted with EA (5 ml*3). The organic layer was dried over Na₂SO₄, filtered and concentrated. The residue was purified by flash silica chromatography, elution gradient 0 to 10% MeOH in DCM to give the desired compound as a yellow solid 26 (185 mg, 88% yield). LC-MS, ES⁻ (m/z): 389.21 (M-1).

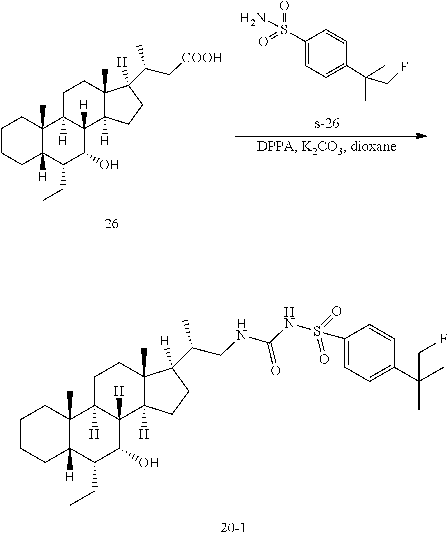

Step 2: Synthesis of Compound 20-1. To a solution of 26 (50.0 mg, 0.128 mmol) in dioxane (1 mL) was added K₂CO₃ (53 mg, 0.384 mmol), s-26 (36 mg, 0.154 mmol) and DPPA (42 mg, 0.154 mmol) under argon atmosphere. The mixture was stirred at 85° C. for 3 h, quenched with brine and extracted with DCM (2*5 mL). The combined organic layer was dried over Na₂SO₄, filtered and concentrated in vacuo. The crude product was purified by silica gel chromatography to give the desired product 20-1 (7.3 mg, 9% yield). LC-MS, ES⁻ (m/z): 617.39 (M-1). ¹H NMR (400 MHz, Chloroform-d) δ 7.80 (dd, J=13.8, 8.4 Hz, 2H), 7.43-7.32 (m, 2H), 6.48 (s, 1H), 3.59 (d, J=2.8 Hz, 1H), 3.25 (s, 1H), 3.17-3.08 (m, 1H), 3.02 (d, J=2.4 Hz, 1H), 2.90 (d, J=2.3 Hz, 1H), 1.98-1.57 (m, 9H), 1.53-0.85 (m, 31H), 0.64 (s, 3H).

### Example 112. Synthesis of Compound 20-2

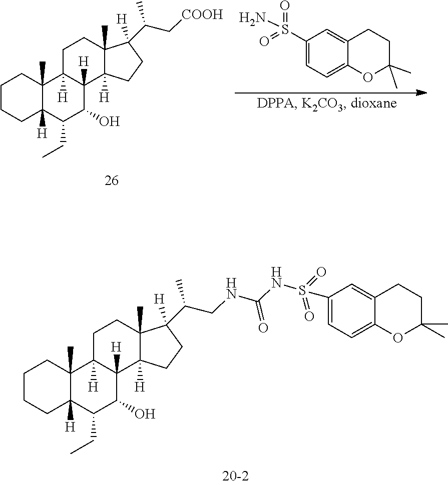

To a solution of 26 (50.0 mg, 0.128 mmol) in dioxane (1 mL) was added K₂CO₃ (53 mg, 0.384 mmol), s-29 (37 mg, 0.154 mmol) and DPPA (42 mg, 0.154 mmol) under argon atmosphere. The mixture was stirred at 85° C. for 3 h, quenched with brine and extracted with DCM (2*5 mL). The combined organic layer was dried over Na₂SO₄, filtered and concentrated in vacuo. The crude product was purified by silica gel chromatography to give the desired product 20-2 (28 mg, 35% yield). LC-MS, ES⁻ (m/z): 627.40 (M-1). 1H NMR (400 MHz, Chloroform-d) δ 7.79 (d, J=2.4 Hz, 1H), 7.74-7.69 (m, 1H), 7.39 (s, 1H), 6.84 (d, J=8.7 Hz, 1H), 3.71-3.64 (m, 1H), 3.38-3.09 (m, 2H), 2.96 (d, J=21.6 Hz, 2H), 2.00-1.06 (m, 33H), 0.94 (d, J=6.6 Hz, 3H), 0.90-0.85 (m, 6H), 0.64 (s, 3H).

### Example 113. Synthesis of Compound 20-3

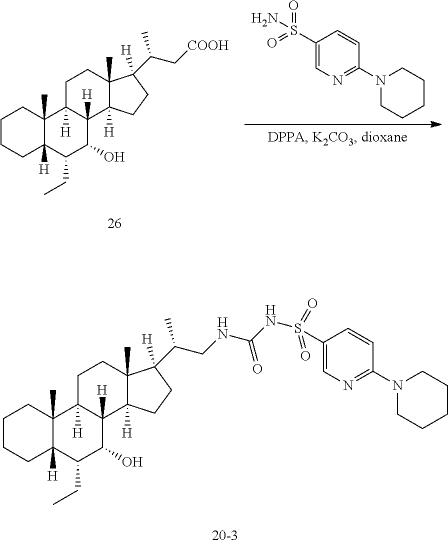

To a solution of 26 (50.0 mg, 0.128 mmol) in dioxane (1 mL) was added K₂CO₃ (53 mg, 0.384 mmol), s-30 (37 mg, 0.154 mmol) and DPPA (42 mg, 0.154 mmol) under argon atmosphere. The mixture was stirred at 85° C. for 3 h, quenched with brine and extracted with DCM (5 mL). The combined organic layer was dried over Na₂SO₄, filtered and concentrated in vacuo. The crude product was purified by silica gel chromatography to give the desired product 20-3 (8 mg, 10% yield). LC-MS, ES⁻ (m/z): 627.40 (M-1).

¹H NMR (400 MHz, Chloroform-d) δ 8.65 (dd, J=2.6, 0.6 Hz, 1H), 7.90 (dd, J=9.3, 2.6 Hz, 1H), 7.54 (s, 1H), 6.59 (dd, J=9.4, 0.7 Hz, 1H), 3.70 (t, J=2.7 Hz, 1H), 3.31 (m, 1H), 3.18 (m, 1H), 2.02-1.09 (m, 35H), 0.96-0.87 (m, 9H), 0.66 (s, 3H).

### Example 114. Synthesis of Compound 21-1

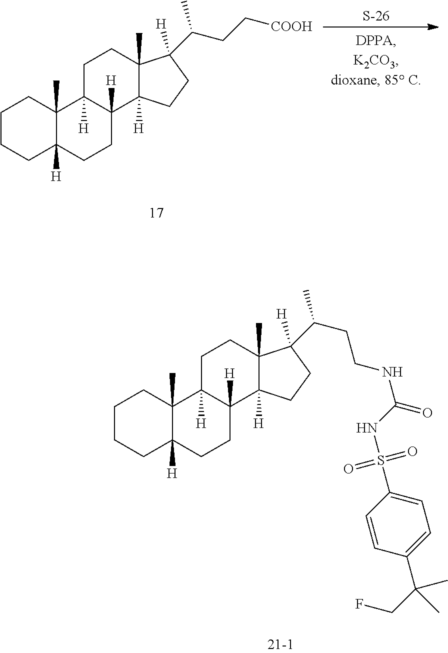

To a solution of compound 17 in dioxane was added sulfonamide intermediates S-26 (1 eq), K₂CO₃ (3 eq) and DPPA (1.2 eq) in return under Ar₂ at rt, then heated to 85° C. for 3 h. The mixture was washed with H₂O, brine, dried over anhydrous Na₂SO₄, and concentrated in vacuo. The residue was purified by silica gel column chromatography to obtain 21-1.

### Example 115. Synthesis of Compound 22-1

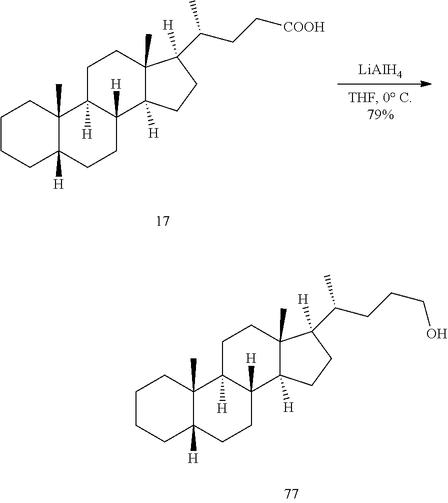

Step 1: Synthesis of compound 77. To a solution of 17 (12.7 mg) in THF (1 mL) at 0° C. was added LiAlH₄ (5.0 mg), and the mixture was stirred at 0° C. for 1.5 h. The mixture was concentrated, and the crude product was purified by silica gel chromatography to give the desired product 77 (9.7 mg, 79%). ¹H NMR (400 MHz, Chloroform-d) δ 3.64-3.58 (m, 2H), 0.92 (d, J=7.4 Hz, 6H), 0.65 (s, 3H).

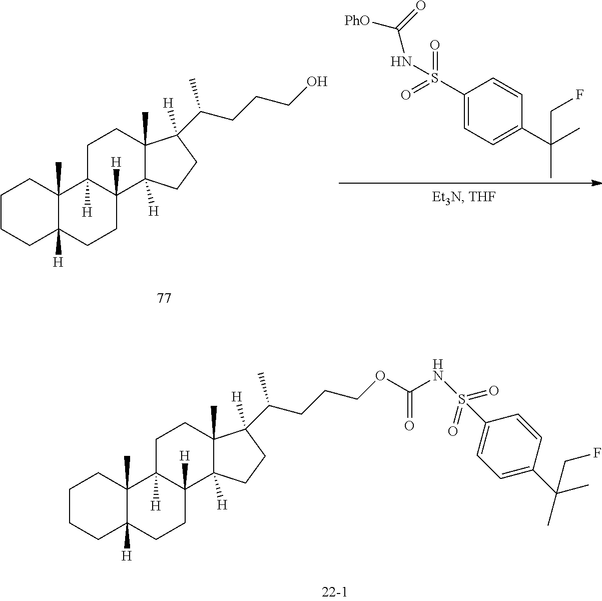

Step 2: Synthesis of compound 22-1. Compound 1D (51 mg, 0.146 mmol) was added to a solution of compound 77 (46 mg, 0.133 mmol) and TEA (40 mg, 0.4 mmol) in dry THF (2 mL) at rt. The reaction was quenched with H₂O (3 mL) and diluted with DCM (3 mL). The layers were separated and the aqueous layer was extracted with DCM (2*3 mL), dry with anhydrous Na₂SO₄, evaporate the solvent. The crude product was purified by silica gel chromatography to give the desired product 22-1 (25.2 mg, 29% yield). LC-MS, ES⁺ (m/z): 604.97 (M+1).

### Example 116. Synthesis of Compound 23-1

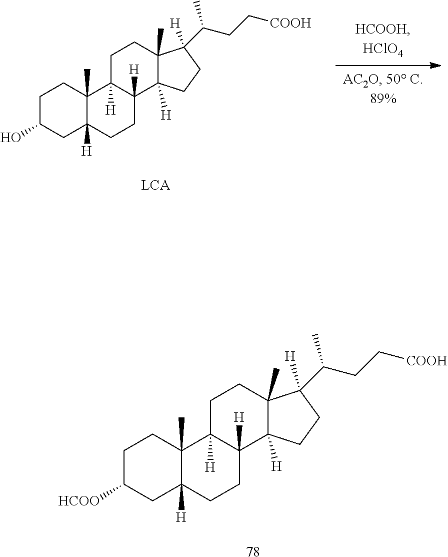

Step 1: Synthesis of compound 78. A mixture of LCA (26.67 g), HCOOH (48 mL, 92% in water) and HClO₄ (12.5 mL) was stirred at 50° C. overnight. Then the mixture was cooled to 40° C., followed by slow addition of Ac₂O (25 mL), and the mixture was stirred for another 10 min. The mixture was cooled to room temperature, quenched with water, and extracted with ether. The combined organic layers were dried over anhydrous Na₂SO₄, filtered, and concentrated in vacuo. The crude product was purified by silica gel chromatography to give the desired product 78 (22 g, 89%).

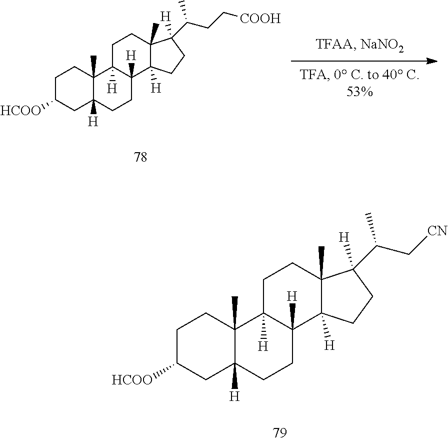

Step 2: Synthesis of compound 79. An ice-cooling mixture of 78 (9.66 g), TFA (50 mL) and TFAA (10 mL) was stirred until the solution become clear, and then NaNO₂ (1.8 g) was added in small portions at 0° C. The resulted mixture was stirred at 0° C. for 1 h, and then the mixture was warmed to 40° C., and stirred for 1 h. After cooling to room temperature, the mixture was neutralized with 1M NaOH solution, and extracted with EA. The combined organic layers were dried over anhydrous Na₂SO₄, filtered, and concentrated in vacuo. The crude product was purified by silica gel chromatography to give the desired product 79 (4.75 g, 53%).

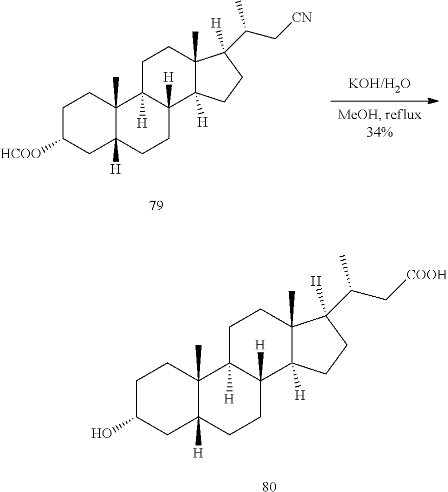

Step 3: Synthesis of compound 80. To a solution of 79 (6.4 g) in MeOH (240 mL) was added KOH (12 g) in water (240 mL), and the mixture was refluxed for 24 h. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 80 (2.12 g, 34%). ¹H NMR (400 MHz, Chloroform-d) δ 3.67-3.58 (m 1H), 1.02 (d, J=6.5 Hz, 3H), 0.92 (s, 3H), 0.68 (s, 3H).

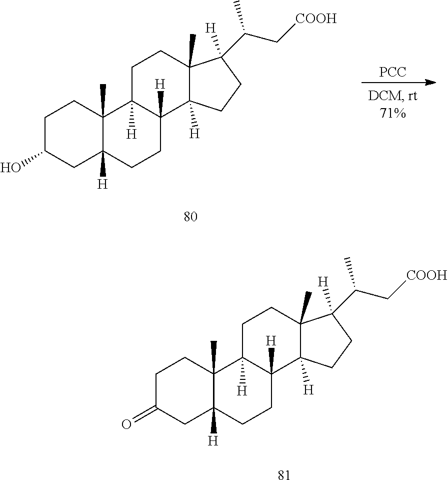

Step 4: Synthesis of compound 81. To a solution of 80 (1.13 g, 3.1 mmol) in DCM (40 mL) was added silica gel (2.1 g) and PCC (1.03 g, 4.73 mmol), and the mixture was stirred at room temperature overnight. The mixture was concentrated, and the crude product was purified by silica gel chromatography (PE:EA=10:1˜5:1) to give the desired product 81 (793 mg, 71%). LC-MS, ES⁺ (m/z): 361.58 (M+1).

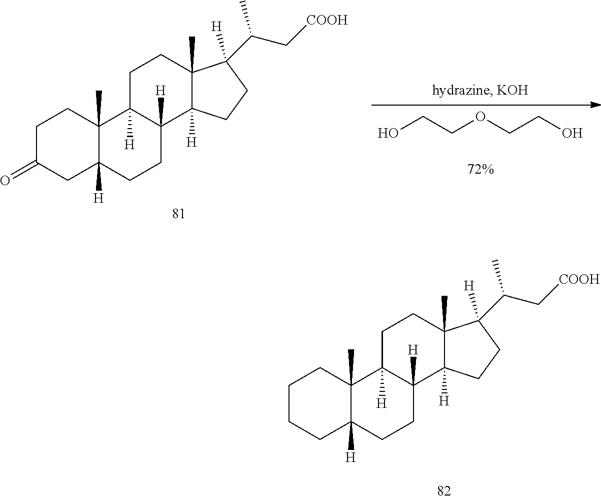

Step 5: Synthesis of compound 82. To a solution of 81 (600 mg, 1.66 mmol) in 2,2′-oxybis(ethan-1-ol) (20 mL) was added KOH (2.3 g) and hydrazine (2.5 mL), and the mixture was stirred at 120° C. for 2 h, followed by 180° C. overnight. The mixture was cooled to room temperature, quenched with water, acidified by 1 M HCl, and extracted with EA. The combined organic layers were dried over anhydrous Na₂SO₄, filtered, and concentrated in vacuo. The crude product was purified by silica gel chromatography to give the desired product 82 (415 mg, 72%). LC-MS, ES⁺ (m/z): 347.68 (M+1).

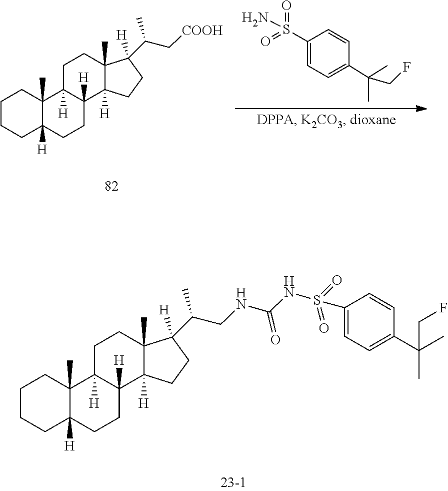

Step 6: Synthesis of compound 23-1. To a solution of 82 (50.0 mg, 0.1443 mmol) in dioxane (1 mL) was added K₂CO₃ (53 mg, 0.384 mmol), s-26 (36 mg, 0.154 mmol) and DPPA (42 mg, 0.154 mmol) under argon atmosphere. The mixture was stirred at 85° C. for 3 h, quenched with brine and extracted with DCM (5 mL). The combined organic layer was dried over Na₂SO₄, filtered and concentrated in vacuo. The crude product was purified by silica gel chromatography to give the desired product 23-1 (43.13 mg, 52% yield). LC-MS, ES⁺ (m/z): 575.89 (M+1).

### Example 117. Synthesis of Compound 24-1

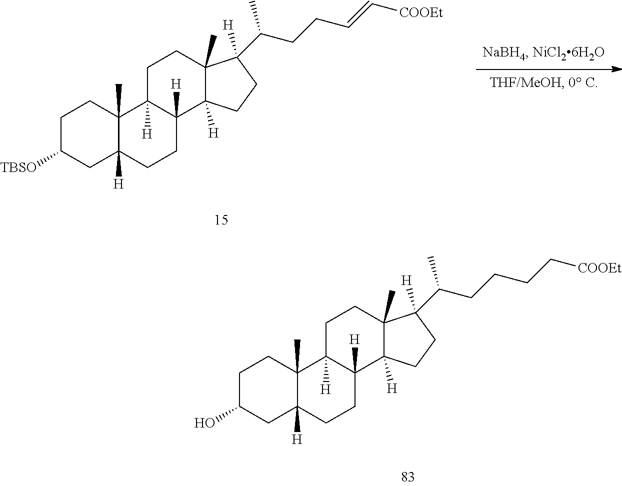

Step 1: Synthesis of compound 83. To a solution of 15 (4.3 g) in MeOH/THF (3:1, 80 mL) at 0° C. was added NiCl₂·6H₂O (1.0 g) and NaBH₄ (1.0 g). The mixture was stirred at 0° C. for 1 h. Then the mixture was quenched with water, and most of the solvent was removed. The residue was extracted with EA, and the combined organic layers were dried over anhydrous Na₂SO₄, filtered, and concentrated in vacuo. The crude product was dissolved in MeOH (180 mL), and 2 drops of concentrated HCl was added. Then the mixture was stirred at room temperature for 1 h, concentrated in vacuo to give the desired product 83 (3.6 g), which was used in the next step without further purification.

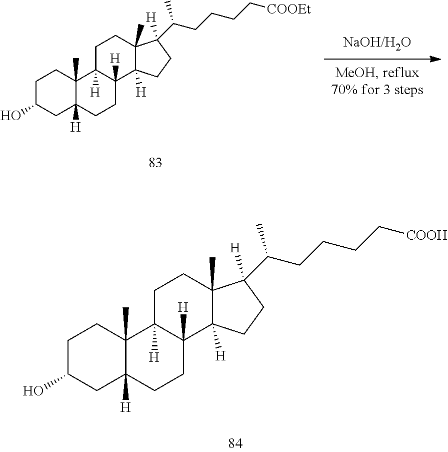

Step 2: Synthesis of compound 84. To a solution of 83 (3.53 g) in MeOH (80 mL) was added NaOH (3.3 g) in water (80 mL), and the mixture was refluxed overnight. After cooling to room temperature, the reaction mixture was acidified with 1M HCl. Solvent was removed, and the residue was extracted with EA. The combined organic layers were dried over anhydrous Na₂SO₄, filtered, and concentrated in vacuo. The crude product was purified by silica gel chromatography to give the desired product 84 (2.23 g, 70% for 3 steps from compound 13). ¹H NMR (400 MHz, Chloroform-d) δ 3.67-3.58 (m, 1H), 2.35 (t, J=7.4 Hz, 2H), 0.92 (s, 3H), 0.89 (d, J=6.5 Hz, 3H), 0.64 (s, 3H).

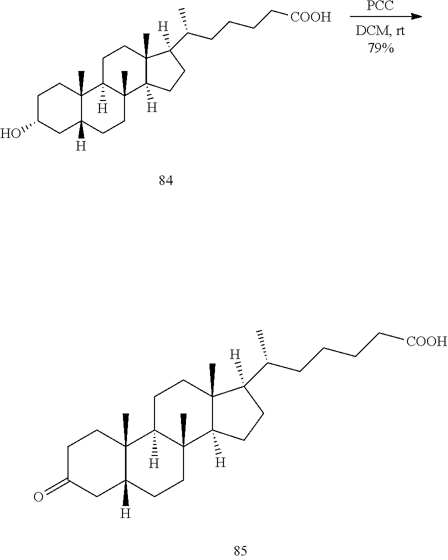

Step 3: Synthesis of compound 85. To a solution of 84 (1.3 g, 3.21 mmol) in DCM (400 mL) was added silica gel (2.5 g) and PCC (1.05 g, 4.815 mmol), and the mixture was stirred at room temperature overnight. The mixture was concentrated, and the crude product was purified by silica gel chromatography (PE:EA=10:1˜5:1) to give the desired product 85 (1.02 g, 79%). LC-MS, ES⁻ (m/z): 403.69 (M-1).

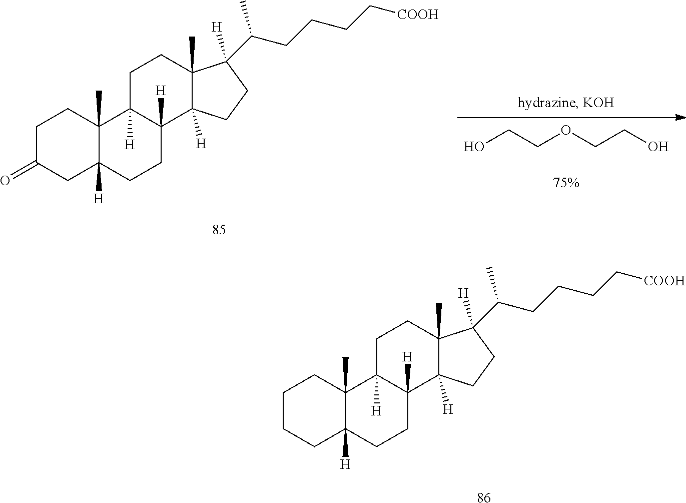

Step 4: Synthesis of compound 86. To a solution of 85 (500 mg, 1.242 mmol) in 2,2′-oxybis(ethan-1-ol) (20 mL) was added KOH (2.3 g) and hydrazine (2.5 mL), and the mixture was stirred at 120° C. for 2 h, followed by 180° C. overnight. The mixture was cooled to room temperature, quenched with water, acidified by 1 M HCl, and extracted with EA. The combined organic layers were dried over anhydrous Na₂SO₄, filtered, and concentrated in vacuo. The crude product was purified by silica gel chromatography to give the desired product 86 (362 mg, 75%). LC-MS, ES⁻ (m/z): 387.59 (M-1).

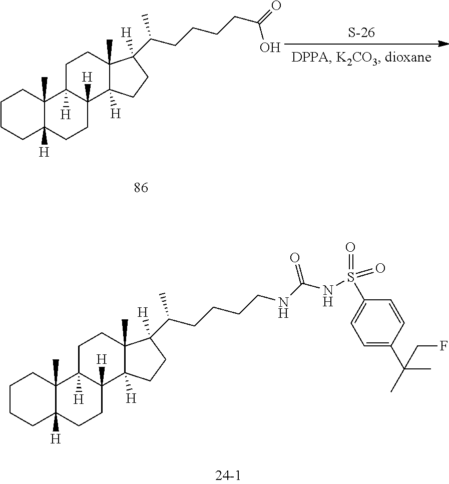

Step 5: Synthesis of compound 24-1. To a solution of 86 (50.0 mg, 0.129 mmol) in dioxane (1 mL) was added K₂CO₃ (53 mg, 0.384 mmol), s-26 (36 mg, 0.154 mmol) and DPPA (42 mg, 0.154 mmol) under argon atmosphere. The mixture was stirred at 85° C. for 3 h, quenched with brine and extracted with DCM (5 mL). The combined organic layer was dried over Na₂SO₄, filtered and concentrated in vacuo. The crude product was purified by silica gel chromatography to give the desired product 24-1 (79.4 mg, 45% yield). LC-MS, ES⁺ (m/z): 617.99 (M+1).

### Example 118. Provided Technologies can Modulate FXR Activities

Among other things, the present disclosure provides technologies, e.g., compounds, compositions, methods, etc., for modulating FXR activities. In some embodiments, provided technologies modulate expression of one or more nucleic acids, e.g., genes, up- or down-regulated upon FXR activation. In some embodiments, provided technologies modulate translocation of FXR to a cell nucleus. In some embodiments, provided technologies increases levels of FXR translocation, e.g., compared to absence of provided compounds or in the presence of a reference compound (e.g., a bile acid) under comparable conditions (e.g., at a comparable concentration). In some embodiments, provided technologies promotes formation of FXR dimerization (e.g., formation of a heterodimer with RXR). In some embodiments, the present disclosure promotes FXR binding to a hormone response element. As those skilled in the art appreciate, various technologies are available for assessment of FXR activation in accordance with the present disclosure. A useful assay is described below as an example. Human FXR (NR1H4) agonist assessment (see, e.g., Yu et al. eLife 2019; 8: e48431): HEK293T cells stably expressing human FXR and containing FXR response element (FXRE)-NanoLuc reporter were cultured in DMEM medium supplemented with 10% FBS and 1% PS, at 37° C. in a humidified atmosphere with 5% CO₂. Cells were detached by centrifuging (1000 rpm, 3 min). The pellet was re-suspended in medium, and cell density was adjusted to about 1×10⁶ cells/mL. 100 μL suspension was seeded to each well of a white-walled, clear-bottom 96-well microplates (cell plate). Cells in microplates were cultured overnight.

In the following day, cells were grown to higher than 95% density in each well. Culture medium was replaced, and compounds were added to the wells. 0.01% DMSO (v/v) served as negative control. Plates were incubated at 37° C. in 5% CO₂ for 24 hr.

In the following day, a 10 μL aliquot of cell culture medium was removed from each well and combined with 40 μL culture medium plus 50 mL assay buffer (containing 20 mM of the luciferase substrate coelenterazine). After 5 min incubation, luminescence was measured using an EnVision plate reader (PerkinElmer).

In some embodiments, gradient diluted solutions of several concentrations were used to treat cells and corresponding responses were used to fit sigmoid function and to derive EC₅₀ in Origin. Certain results are presented in Table E1 below as examples. +++: EC50<=1.0 μM; ++: 1.0 μM<EC50<10 μM; +: EC50>=10 μM. Efficacy (maximum measured induction of NanoLuc) was normalized to CDCA set as 100%; A: efficacy >400%; B: 300%<efficacy <=400%; C: 100%<efficacy <=300%; D: efficacy <=100%. In some embodiments, assessed concentrations were or included about 10⁻⁴, 10^{-4.5}, 10⁻⁵, 10^{-5.5}, 10⁻⁶, 10^{-6.5}, 10⁻⁷, 10⁻⁸, and/or 10⁻⁹ M; for example, in many instances, assessed concentrations included about 10⁻⁵, 10⁻⁶, 10⁻⁷ and 10⁻⁸ M. In some embodiments, assessed contractions were or included 4.0, 1.0, 0.25, 0.0625, 0.0156, 0.00391, 0.00098, 0.000244, 0.000061, and/or 0.0000153 uM. Among other things, it was observed that various compounds can activate FXR at one or more concentrations (e.g., about or at least about EC10, EC20, EC30, EC40, EC50, EC60, EC70, EC80, EC90, or EC100 of a compound or a reference compound for FXR) at various levels (e.g., about or at least about 20%, 30%, 40%, 50%, 60%, 70%, 75%, 80%, 90%, 95%, 100%, 120%, 150%, 200%, 250%, 300%, 400%, 500%, or 1000% of a reference compound observed at the same concentration; as confirmed herein, in many instances, about or at least about 100%, 150%, 200%, 250%, 300%, or 400% of a reference compound observed at the same concentration). In some embodiments, a concentration is about 10⁻⁴, 10^{-4.5}, 10⁻⁵, 10^{-5.5}, 10⁻⁶, 10^{-6.5}, 10⁻⁷, 10⁻⁸ and/or 10⁻⁹ M. In some embodiments, a concentration is about 10⁻⁴ M. In some embodiments, a concentration is about 10^{-4.5} M. In some embodiments, a concentration is about 10⁻⁵ M. In some embodiments, a concentration is about 10^{-5.5} M. In some embodiments, a concentration is about 10⁻⁶ M. In some embodiments, a concentration is about 10^{-6.5} M. In some embodiments, a concentration is about 10⁻⁷ M. In some embodiments, it was observed some compounds provided FXR activation with EC50 about or even lower than 10⁻⁸ M. Various compounds useful as reference compounds are described herein. For example, in some embodiments, a reference compound is DCA. In some embodiments, a reference compound is OCA. In some embodiments, a reference compound is CDCA. In some embodiments, a compound does not have a —OH group for R¹ or R^{1a}, and a reference compound has one of R¹ and R^{1a} being —OH as in OCA but is otherwise identical.

TABLE El
Provided technologies can inhibit FXR.

| | EC₅₀ | | | EC₅₀ | |
|---|---|---|---|---|---|
| Compound | (μM) | Efficacy | Compound | (μM) | Efficacy |
| CDCA | ++ | Set as 100% | 5-24 | +++ | B |
| OCA | +++ | A | 5-25 | ++ | C |
| RC-1 | ++ | B | 5-26 | +++ | A |
| RC-2 | ++ | B | 5-27 | +++ | A |
| RC-3 | ++ | B | 5-29 | +++ | A |
| RC-4 | +++ | A | 5-30 | +++ | A |
| RC-5 | ++ | B | 6-1 | +++ | B |
| RC-6 | ++ | B | 6-2 | ++ | C |
| RC-7 | ++ | C | 6-3 | ++ | C |
| 1-1 | ++ | C | 6-4 | + | C |
| 1-2 | + | D | 6-5 | ++ | C |
| 1-3 | + | C | 7-1 | + | C |
| 1-4 | ++ | C | 7-2 | +++ | B |
| 1-5 | + | D | 7-3 | +++ | C |
| 1-6 | + | D | 8-1 | ++ | B |
| 1-7 | ++ | C | 8-2 | +++ | B |
| 10 | ++ | C | 8-3 | +++ | C |
| 16 | + | C | 8-4 | +++ | B |
| 17 | ++ | B | 8-5 | +++ | C |
| 21 | ++ | C | 9-1 | +++ | B |
| 55 | +++ | C | 9-2 | +++ | B |
| 73 | ++ | A | 9-3 | +++ | B |
| 77 | ++ | B | 9-4 | +++ | C |
| 80 | ++ | A | 9-5 | +++ | C |
| 84 | ++ | B | 9-6 | +++ | C |
| 2-1 | + | C | 9-7 | ++ | A |
| 2-2 | + | C | 10-1 | +++ | B |
| 2-3 | ++ | C | 10-2 | +++ | C |
| 2-4 | + | C | 10-3 | +++ | C |
| 2-6 | ++ | B | 10-4 | +++ | C |
| 3-1 | ++ | C | 11-1 | + | C |
| 3-2 | + | C | 11-2 | + | D |
| 3-3 | + | C | 11-3 | +++ | C |
| 4-1 | +++ | B | 11-4 | +++ | B |
| 4-2 | +++ | B | 12-1 | +++ | C |
| 4-3 | ++ | B | 12-2 | +++ | A |
| 5-1 | +++ | B | 13-1 | +++ | C |
| 5-2 | +++ | A | 13-2 | +++ | A |
| 5-3 | ++ | C | 14-1 | + | C |
| 5-4 | +++ | B | 14-2 | + | C |
| 5-5 | +++ | C | 14-3 | +++ | C |
| 5-6 | +++ | C | 14-4 | +++ | C |
| 5-7 | +++ | B | 15-1 | ++ | B |
| 5-8 | +++ | C | 15-2 | +++ | C |
| 5-9 | +++ | B | 15-3 | ++ | C |
| 5-10 | +++ | C | 16-1 | ++ | B |
| 5-11 | ++ | C | 16-2 | +++ | C |
| 5-12 | ++ | C | 17-1 | +++ | A |
| 5-13 | +++ | C | 17-2 | +++ | B |
| 5-14 | +++ | C | 18-1 | +++ | A |
| 5-15 | +++ | C | 18-2 | +++ | A |
| 5-16 | +++ | C | 18-3 | +++ | A |
| 5-17 | +++ | C | 19-1 | +++ | B |
| 5-18 | +++ | B | 19-2 | +++ | B |
| 5-19 | +++ | C | 19-3 | +++ | A |
| 5-20 | +++ | C | 20-1 | +++ | A |
| 5-21 | +++ | C | 20-2 | ++ | B |
| 5-22 | +++ | C | 20-3 | ++ | B |
| 5-23 | +++ | A | 26 | + | C |
| 53 | +++ | C | 2-5 | ++ | A |
| 21-1 | ++ | B | 22-1 | ++ | B |
| 23-1 | ++ | B | 24-1 | ++ | B |

CDCA: chenodeoxycholic acid; OCA: 6-ethylchenodeoxycholic acid.

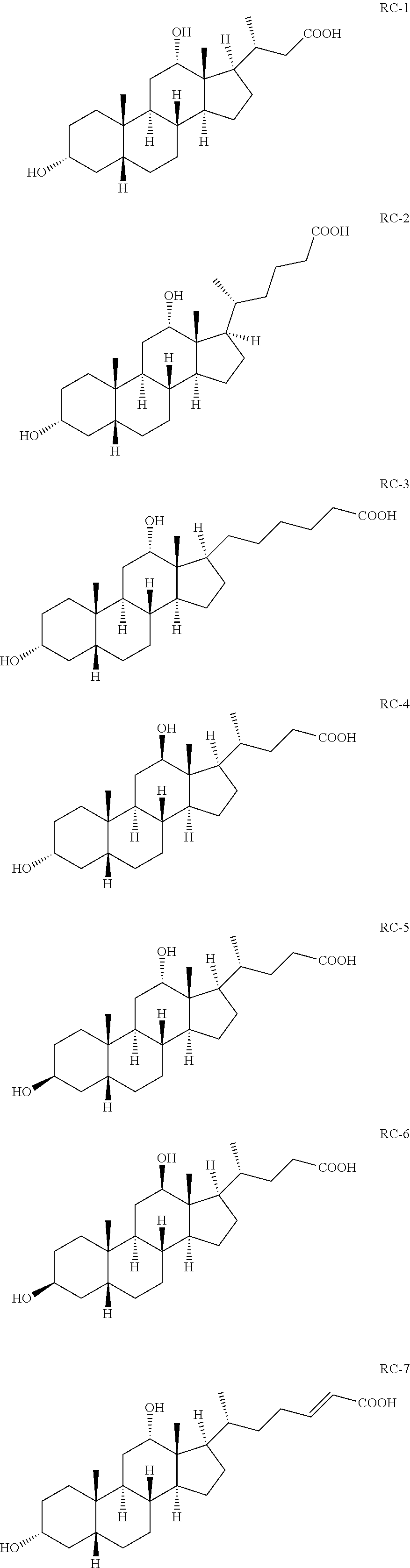

(SEQ ID NO: 1)
ATGGGATCAAAAATGAATCTCATTGAACATTCCCATTTACCTACCACAGA
TGAATTTTCTTTTTCTGAAAATTTATTTGGTGTTTTAACAGAACAAGTGG
CAGGTCCTCTGGGACAGAACCTGGAAGTGGAACCATACTCGCAATACAGC
AATGTTCAGTTTCCCCAAGTTCAACCACAGATTTCCTCGTCATCCTATTA
TTCCAACCTGGGTTTCTACCCCCAGCAGCCTGAAGAGTGGTACTCTCCTG
GAATATATGAACTCAGGCGTATGCCAGCTGAGACTCTCTACCAGGGAGAA
ACTGAGGTAGCAGAGATGCCTGTAACAAAGAAGCCCCGCATGGGCGCGTC
AGCAGGGAGGATCAAAGGGGATGAGCTGTGTGTTGTTTGTGGAGACAGAG
CCTCTGGATACCACTATAATGCACTGACCTGTGAGGGGTGTAAAGGTTTC
TTCAGGAGAAGCATTACCAAAAACGCTGTGTACAAGTGTAAAAACGGGGG
CAACTGTGTGATGGATATGTACATGCGAAGAAAGTGTCAAGAGTGTCGAC
TAAGGAAATGCAAAGAGATGGGAATGTTGGCTGAATGTATGTATACAGGC
TTGTTAACTGAAATTCAGTGTAAATCTAAGCGACTGAGAAAAAATGTGAA
GCAGCATGCAGATCAGACCGTGAATGAAGACAGTGAAGGTCGTGACTTGC
GACAAGTGACCTCGACAACAAAGTCATGCAGGGAGAAAACTGAACTCACC
CCAGATCAACAGACTCTTCTACATTTTATTATGGATTCATATAACAAACA
GAGGATGCCTCAGGAAATAACAAATAAAATTTTAAAAGAAGAATTCAGTG
CAGAAGAAAATTTTCTCATTTTGACGGAAATGGCAACCAATCATGTACAG
GTTCTTGTAGAATTCACAAAAAAGCTACCAGGATTTCAGACTTTGGACCA
TGAAGACCAGATTGCTTTGCTGAAAGGGTCTGCGGTTGAAGCTATGTTCC
TTCGTTCAGCTGAGATTTTCAATAAGAAACTTCCGTCTGGGCATTCTGAC
CTATTGGAAGAAAGAATTCGAAATAGTGGTATCTCTGATGAATATATAAC
ACCTATGTTTAGTTTTTATAAAAGTATTGGGGAACTGAAAATGACTCAAG
AGGAGTATGCTCTGCTTACAGCAATTGTTATCCTGTCTCCAGATAGACAA
TACATAAAGGATAGAGAGGCAGTAGAGAAGCTTCAGGAGCCACTTCTTGA
TGTGCTACAAAAGTTGTGTAAGATTCACCAGCCTGAAAATCCTCAACACT
TTGCCTGTCTCCTGGGTCGCCTGACTGAATTACGGACATTCAATCATCAC
CACGCTGAGATGCTGATGTCATGGAGAGTAAACGACCACAAGTTTACCCC
ACTTCTCTGTGAAATCTGGGACGTGCAGTGA.

### Amino Acid Sequence:

(SEQ ID NO: 2)
MGSKMNLIEHSHLPTTDEFSFSENLFGVLTEQVAGPLGQNLEVEPYSQY
SNVQFPQVQPQISSSSYYSNLGFYPQQPEEWYSPGIYELRRMPAETLYQ
GETEVAEMPVTKKPRMGASAGRIKGDELCVVCGDRASGYHYNALTCEGC
KGFFRRSITKNAVYKCKNGGNCVMDMYMRRKCQECRLRKCKEMGMLAEC
MYTGLLTEIQCKSKRLRKNVKQHADQTVNEDSEGRDLRQVTSTTKSCRE
KTELTPDQQTLLHFIMDSYNKQRMPQEITNKILKEEFSAEENFLILTEM
ATNHVQVLVEFTKKLPGFQTLDHEDQIALLKGSAVEAMFLRSAEIFNKK
LPSGHSDLLEERIRNSGISDEYITPMFSFYKSIGELKMTQEEYALLTAI
VILSPDRQYIKDREAVEKLQEPLLDVLQKLCKIHQPENPQHFACLLGRL
TELRTFNHHHAEMLMSWRVNDHKFTPLLCEIWDVQ.

### Example 119. Provided Technologies can Modulate TGR5 Activities

In some embodiments, the present disclosure provides technologies, e.g., compounds, compositions, methods, etc., for modulation a GPCR function. In some embodiments, the present disclosure provides technologies for activating a GPCR, e.g., a GPCR responsive to a bile acid or a derivative thereof. In some embodiments, a GPCR is TGR5. In some embodiments, provided technologies modulate expression of one or more nucleic acids, e.g., genes up- or down-regulated upon GPCR, e.g., TGR5, activation. In some embodiments, provided technologies modulate translocation, e.g., internalization, of a GPCR (e.g., TGR5), increase intracellular cAMP, activate a kinase (e.g., a MAP kinase), increase CREB phosphorylation, and/or increase expression of a CREB-regulated nucleic acid (e.g., a gene activated by interactions between a cAMP response element and phosphorylated CREB), compared to absence of provided technologies and/or in the presence of a reference compound (e.g., a bile acid) under comparable conditions (e.g., at a comparable concentration). As those skilled in the art appreciate, various technologies are available for assessment of GPCR (e.g., TGR5) activation, or for assessing if a GPCR receptor is responsive to a provided compound, in accordance with the present disclosure. A useful assay is described below as an example.

TGR5 agonist assessment: HEK293T cells stably expressing human TGR5 and containing cAMP response element (CRE)-NanoLuc reporter (see, e.g., Yu et al. eLife 2019; 8: e48431) were cultured in DMEM medium supplemented with 10% FBS and 1% PS, at 37° C. in a humidified atmosphere with 5% CO₂. Cells were detached by centrifuging (1000 rpm, 3 min). The pellet was re-suspended in medium, and cell density was adjusted to about 1×10⁶ cells/mL. 100 μL suspension was seeded to each well of the white-walled, clear-bottom 96-well microplates (cell plate). Cells in the microplates were cultured overnight.

In the following day, cells were grown to higher than 95% density in each well. Culture medium was replaced, and compounds were added to the wells. 0.01% DMSO (v/v) served as negative control. Plates were incubated at 37° C. in 5% CO₂ for 24 hr.

In the following day, a 10 μL aliquot of cell culture medium was removed from each well and combined with 40 μL culture medium plus 50 mL assay buffer (containing 20 mM of the luciferase substrate coelenterazine). After 5 min incubation, luminescence was measured using an EnVision plate reader (PerkinElmer).

In some embodiments, gradient diluted solutions of several concentrations were used to treat cells and corresponding responses were used to fit sigmoid function and to derive EC50 in Origin.

Among other things, provided technologies can be potent TGR5 agonists. For example, compound 5-15 in one experiment showed an EC₅₀ of 13 nM, which DCA assessed under comparable conditions showed an EC₅₀ of 0.6 μM.

Among other things, it was observed that various compounds can activate TGR5 at one or more concentrations (e.g., about or at least about EC10, EC20, EC30, EC40, EC50, EC60, EC70, EC80, EC90, or EC100 of a compound or a reference compound for TGR5) at various levels (e.g., about or at least about 20%, 30%, 40%, 50%, 60%, 70%, 75%, 80%, 90%, 95%, 100%, 120%, 150%, 200%, 250%, 300%, 400%, 500%, or 1000% of a reference compound observed at the same concentration; as confirmed herein, in many instances, about or at least about 100%, 150%, 200%, 250%, 300%, or 400% of a reference compound observed at the same concentration). In some embodiments, a concentration is about 10⁻⁴, 10^{-4.5}, 10⁻⁵, 10⁻⁵, 10⁻⁶, 10^{-6.5}, 10⁻⁷, 10⁻⁸, and/or 10⁻⁹ M. In some embodiments, a concentration is about 10⁻⁴ M. In some embodiments, a concentration is about 10^{-4.5} M. In some embodiments, a concentration is about 10⁻⁵ M. In some embodiments, a concentration is about 10^{-5.5} M. In some embodiments, a concentration is about 10⁻⁶ M. In some embodiments, a concentration is about 10^{-6.5} M. In some embodiments, a concentration is about 10⁻⁷ M. In some embodiments, it was observed some compounds provided TGR5 activation with EC50 about or even lower than 10⁻⁸ M. Various compounds useful as reference compounds are described herein. For example, in some embodiments, a reference compound is DCA. In some embodiments, a reference compound is OCA. In some embodiments, a reference compound is CDCA. In some embodiments, a compound does not have a —OH group for R¹ or R^{1a}, and a reference compound has one of R¹ and R^{1a} being —OH as in OCA but is otherwise identical.

### Example 120. Provided Technologies can Provide Reduced Side Effects

Various side effects associated with administration of FXR or TGR5 agonists, e.g., bile acids and derivatives thereof, are reported. Among other things, the present disclosure recognizes that various such side effects are associated with MRGPRX4 activation and can be reduced, removed and/or prevented by replacing 3-OH bonded to a moiety A with, e.g., —H, —F, etc. Among other things, provided technologies have low MRGPRX4 activation activity. As demonstrated herein, various compounds showed no MRGPRX4 activation at the highest concentrations assessed.

As those skilled in the art appreciate, various technologies are available for assessment of MRGPRX4 activation in accordance with the present disclosure. A useful assay is described below as an example.

In some embodiments, cells were cultured in poly-D-Lys coated blacked walled, clear-bottom 96-well microplates. HEK293T cells stably expressing MRGPRX4 were cultured in DMEM medium supplemented with 10% FBS and 1% PS, at 37° C. in a humidified atmosphere with 5% CO₂. Cells were detached by centrifuging (1000 rpm, 3 min). The pellet was re-suspended in medium, and cell density was adjusted to about 1×10⁶ cells/mL. 100 μL suspension was seeded to each well of a microplates (cell plate). Cells in the microplates were cultured overnight. In the following day, cells were grown to higher than 95% density in each well.

Ca²⁺ fluorescent dye loading: Fluo-8 AM (Screen Quest Fluo-8 AM Calcium Assay Kit, AAT Bioquest, cat. #: 21083) was diluted in HHBS buffer and adjusted to 2 μM (dye buffer). Medium in each well was changed into 50 μL dye buffer carefully. After incubation for 40-60 min under room temperature, the dye buffer was changed into 50 μL HHBS buffer.

Solution of chemicals (ligands (deoxycholic acid (DCA), positive control) and compounds) were prepared in clear 96-well microplates (compound plate). The concentration of solution should be determined according to the protocol settings and experimental needs (e.g., Log[M]=−4, −5, −6, −7, −8, and −9). Prepare 2× serial dilution series of compounds (at least 75 μL per well).

A fluorescent signal baseline was measured using FLIPR®Tetra for 30 s. Then 50 μL of the chemical solution would be added to the cell plate. The fluorescent signal would be measured for another 150 s. The maximum response was calculated for each well and normalized to the response of positive control (treated with MRGPRX4 ligand (DCA)) to get a relative response.

Activity of each compound was calculated by the fluorescence change (dF/F0). Curves were fitted to obtain EC₅₀ values. In some embodiments, at least 3 repeats were performed to obtain the average values. For assessment of a compound, gradient diluted solutions of different concentrations were used to treat cells and corresponding responses were used to fit sigmoid function and to derive EC₅₀ in Origin. In some embodiments, assessed concentrations were or included about 10⁻⁴, 10^{-4.5}, 10⁻⁵, 10^{-5.5}, 10⁻⁶, 10^{-6.5}, 10⁻⁷, 10⁻⁸, and/or 10⁻⁹ M; for example, in many instances, assessed concentrations included about 10⁻⁵, 10⁻⁶, 10⁻⁷ and 10⁻⁸ M. In some embodiments, assessed concentrations were about 10⁻⁴, 10^{-4.5}, 10⁻⁵, 10^{-5.5}, 10⁻⁶, 10⁻⁷, 10⁻⁸, and 10⁻⁹ M. In some embodiments, assessed concentrations were about 10⁻⁴, 10⁻⁵, 10⁻⁶, 10⁻⁷, 10⁻⁸, and 10⁻⁹ M. In some embodiments, assessed concentrations were about 10⁻⁴, 10^{-4.5}, 10⁻⁵, 10^{-5.5}, 10⁻⁶, and 10⁻⁷ M. In some embodiments, assessed concentrations were about 2000000, 1000000, 500000, 250000, 125000, 62500, 31250, 15625, 7813, 3906 nM. In some embodiments, assessed concentrations were about 100000, 33333, 11111, 3704, 1235, 412, 137, 46, 15, 5 nM. In some embodiments, assessed concentrations were about 30000, 10000, 3333, 1111, 370, 123, 41, 14, 4.6, 1.5 nM. In some embodiments, assessed concentrations were about 10⁻⁴ 10^{-4.5}, 10⁵, 10^{-5.4}, 10^{-5.9}, 10^{-6.4}, 10^{-6.9}, 10^{-7.3}, 10^{-7.8}, and 10^{-8.3} M. In some embodiments, assessed concentrations were about 10⁻⁴, 10^{-4.5}, 10⁻⁵, 10^{-5.4}, 10^{-5.9}, 10^{-6.4}, 10^{-6.9}, 10^{-7.3}, and 10^{-7.8} M. In some embodiments, assessed concentrations were about 10^{-3.7}, 10⁻⁴, 10^{-4.3}, 10^{-4.6}, 10^{-4.9}, 10^{-5.2}, 10^{-5.5}, 10^{-5.8}, 10^{-6.1}, and 10^{-6.4} M. Certain results are presented in Table E2 below as examples. I: EC₅₀<50 μM; II: 50 μM<=EC₅₀<=100 μM; and III: EC₅₀>100 μM. In some embodiments, no activation observed in the tested concentration range (reported as “III”); for example, see data in Tables below.

TABLE E2
Provided technologies can provide
reduced or no MRGPRX4 activation.

| Compound | Activity (EC₅₀ μM) | Compound | Activity (EC₅₀ μM) |
|---|---|---|---|
| DCA | I (5.0) | 5-15 | III |
| OCA | I (37.0) | 5-16 | III |
| RC-1 | I | 5-17 | III |
| RC-2 | I | 5-18 | III |
| RC-3 | I | 5-20 | III |
| RC-4 | I | 5-21 | III |
| RC-5 | II | 5-22 | III |
| RC-6 | I | 5-23 | III |
| RC-7 | I | 5-25 | III |
| 1-1 | III | 5-26 | III |
| 1-2 | II | 5-29 | III |
| 1-3 | I | 5-30 | III |
| 1-4 | III | 6-2 | III |
| 1-5 | II | 6-3 | III |
| 1-6 | III | 6-4 | III |
| 1-7 | I | 6-5 | III |
| 10 | III | 7-1 | III |
| 16 | I | 53 | III |
| 17 | I | 8-2 | III |
| 21 | III | 8-3 | III |
| 26 | III | 8-4 | III |
| 55 | III | 8-5 | III |
| 73 | III | 9-1 | III |
| 77 | III | 9-2 | II |
| 84 | I | 9-3 | III |
| 2-1 | III | 9-4 | III |
| 2-2 | III | 9-5 | III |
| 2-3 | III | 9-6 | III |
| 2-4 | III | 9-7 | III |
| 2-5 | III | 10-1 | III |
| 2-6 | II | 10-2 | III |
| 3-1 | III | 10-3 | III |
| 3-2 | III | 10-4 | III |
| 3-3 | III | 11-2 | III |
| 4-1 | III | 11-3 | III |
| 4-2 | III | 11-4 | III |
| 4-3 | III | 12-1 | III |
| 5-1 | III | 12-2 | III |
| 5-2 | III | 13-1 | III |
| 5-3 | III | 13-2 | III |
| 5-4 | III | 14-1 | III |
| 5-5 | III | 14-2 | III |
| 5-6 | III | 14-3 | III |
| 5-7 | III | 14-4 | III |
| 5-8 | III | 18-1 | III |
| 5-10 | III | 18-2 | III |
| 5-11 | III | 18-3 | III |
| 5-12 | III | 20-1 | III |
| 5-13 | III | 20-2 | III |
| 5-14 | III | 20-3 | III |

DCA: deoxycholic acid; OCA: 6-ethylchenodeoxycholic acid.

As demonstrated herein, various provided compounds do not activate MRGPRX4 at high concentrations, in some embodiments, even at the highest concentrations tested. In some embodiments, provided technology provides high ratios of EC₅₀ (MRGPRX4)/EC₅₀ (FXR) compared to a reference technology. In some embodiments, provided technology provides high ratios of EC₅₀ (MRGPRX4)/EC₅₀ (TGR5) compared to a reference technology. Among other things, provided technologies can provide various benefits and advantages, e.g., reduced side effects, improved therapeutic indexes, therapeutic windows, regimens, administration, and/or clinical outcomes, etc., compared to a reference technology. In some embodiments, a reference technology is or comprises a bile acid or derivative thereof that has 3-OH. In some embodiments, a reference technology is or comprises a natural bile acid. In some embodiments, a reference technology is or comprises DCA. In some embodiments, a reference technology is or comprises CDCA. In some embodiments, a reference technology is or comprises cholic acid. In some embodiments, a reference technology is or comprises a bile acid derivative which has a 3-substitution. In some embodiments, a reference technology is or comprises a bile acid derivative which has a 3-OH. In some embodiments, a reference technology is or comprises obeticholic acid.

Additional data are provided below as examples. Certain data were utilized to generate the results in the Table E2 above. Among other things, it was confirmed that provided technologies provided reduced or no activation of MRGPRX4 compared to various reference compounds. In some embodiments, EC50 values for MRGPRX4 of various provided compounds are higher than a reference compound (e.g., 2, 3, 4, 5, 6, 7, 8, 9, 10, 20, 50, 100, 200, 500, 1000 or more fold of a reference compound). In some embodiments, EC50 values for MRGPRX4 of various provided compounds are more than the highest concentrations assessed in an assay, e.g., 10⁻⁴ M. As demonstrated herein, activation of MRGPRX4 by various provided compounds at various concentrations, if any, is much lower than various reference compounds such as DCA, OCA, etc. at the same or comparable concentrations. In some embodiments, activation of MRGPRX4 by various provided compounds at one or more concentrations is independently no more than about 1%, 5%, 10%, 20%, 25%, 30%, 33%, 40%, 50%, 60%, 70%, 75%, or 80% (in some embodiments, about or no more than about 50%; in some embodiments, about or no more than about 10%; in some embodiments, about or no more than about 5%) of a reference compound, e.g., DCA, OCA, etc., at the same concentrations, respectively. In some embodiments, at various concentrations MRGPRX4 activation by a reference compound was observed while no activation by a provided compound was observed. In some embodiments, a concentration is a concentration assessed herein (e.g., about 10⁻⁶, 10^{-5.5}, 10⁻⁵, 10^{-4.5}, or 10⁻⁴ M, a concentration in a Table, etc.) wherein activation of MRGPRX4 by a reference compound (e.g., DCA, OCA, etc.) is observed. In some embodiments, a concentration is about EC10, EC20, EC30, EC50, EC80, etc., of a reference compound for MRGPRX4. In some embodiments, a concentration is EC50 of a reference compound for MRGPRX4. In some embodiments, a concentration is about EC10, EC20, EC30, EC50, EC80, etc., of a provided compound for MRGPRX4. In some embodiments, a concentration is about, or about 2, 5, 10, 20, 50, 100, 200, 500, or more fold of, EC10, EC20, EC30, EC50, EC80, etc., of a provided compound for FXR. In some embodiments, a concentration is about, or about 2, 5, 10, 20, 50, 100, 200, 500 or more fold (e.g., about 10 fold or more, about 50 fold or more, about 100 or more, about 500 or more fold, etc.) of, EC10, EC20, EC30, EC50, EC80, etc., of a provided compound for TGR5. In some embodiments, a concentration is about or at least about a therapeutically effective concentration (e.g., delivered by a therapeutically effective amount, dosage or dosage regimen). In some embodiments, one or more or all concentrations are independently about 10⁻⁵, 10^{-4.5}, or 10⁻⁴ M. In some embodiments, a concentration is about 10⁻⁴ M. In some embodiments, a concentration is about 10^{-4.5} M. In some embodiments, a concentration is about 10⁻⁵ M. Various compounds useful as reference compounds are described herein. For example, in some embodiments, a reference compound is DCA. In some embodiments, a reference compound is OCA. In some embodiments, a reference compound is CDCA. In some embodiments, a compound does not have a —OH group for R¹ or R^{1a}, and a reference compound has one of R¹ and R^{1a} being —OH as in OCA but is otherwise identical. In some embodiments, one or more or all concentrations are independently about 10⁻⁵, 10^{-4.5}, or 10⁻⁴ M, and a reference compound is OCA. In some embodiments, one or more or all concentrations are independently about 10⁶, 10^{-5.5}, 10⁻⁵, 10^{-4.5}, or 10⁻⁴ M, and a reference compound is DCA.

TABLE E3
Reduced or no activation of MRGPRX4 by provided technologies.

| Conc. (Log[M]) | | | | | | | |
|---|---|---|---|---|---|---|---|
| −4 | −4.5 | −5 | −5.5 | −6 | −7 | −8 | −9 |

E3-1:

| | | | | | | | | | |
|---|---|---|---|---|---|---|---|---|---|
| OCA | Average | 0.766 | 0.188 | 0.023 | −0.031 | −0.032 | −0.032 | −0.029 | −0.033 |
| | SD | 0.098 | 0.068 | 0.018 | 0.015 | 0.011 | 0.014 | 0.013 | 0.007 |
| | SEM | 0.056 | 0.039 | 0.010 | 0.009 | 0.006 | 0.008 | 0.007 | 0.004 |
| 2-2 | Average | 0.079 | 0.002 | −0.037 | −0.037 | −0.028 | −0.027 | −0.030 | −0.023 |
| | SD | 0.003 | 0.006 | 0.005 | 0.002 | 0.011 | 0.005 | 0.005 | 0.022 |
| | SEM | 0.002 | 0.004 | 0.003 | 0.001 | 0.007 | 0.003 | 0.003 | 0.012 |
| 5-3 | Average | 0.297 | 0.031 | 0.001 | −0.012 | −0.033 | −0.011 | −0.012 | 0.002 |
| | SD | 0.030 | 0.030 | 0.028 | 0.017 | 0.008 | 0.022 | 0.004 | 0.011 |
| | SEM | 0.017 | 0.017 | 0.016 | 0.010 | 0.005 | 0.012 | 0.002 | 0.006 |
| 73 | Average | 0.098 | 0.042 | −0.031 | −0.031 | −0.020 | −0.023 | −0.023 | −0.014 |
| | SD | 0.025 | 0.008 | 0.012 | 0.008 | 0.005 | 0.011 | 0.011 | 0.019 |
| | SEM | 0.014 | 0.005 | 0.007 | 0.005 | 0.003 | 0.006 | 0.007 | 0.011 |
| 2-3 | Average | 0.074 | 0.027 | −0.036 | −0.031 | −0.031 | −0.008 | −0.023 | −0.017 |
| | SD | 0.014 | 0.025 | 0.006 | 0.018 | 0.012 | 0.008 | 0.011 | 0.021 |
| | SEM | 0.008 | 0.015 | 0.003 | 0.010 | 0.007 | 0.004 | 0.007 | 0.012 |
| 77 | Average | 0.067 | 0.014 | −0.029 | −0.023 | −0.017 | −0.009 | −0.014 | −0.012 |
| | SD | 0.008 | 0.015 | 0.011 | 0.001 | 0.015 | 0.001 | 0.003 | 0.012 |
| | SEM | 0.005 | 0.008 | 0.006 | 0.001 | 0.009 | 0.000 | 0.002 | 0.007 |
| 4-1 | Average | 0.057 | 0.035 | −0.037 | −0.013 | 0.002 | 0.009 | 0.004 | 0.005 |
| | SD | 0.017 | 0.015 | 0.006 | 0.039 | 0.006 | 0.018 | 0.006 | 0.003 |
| | SEM | 0.010 | 0.009 | 0.004 | 0.022 | 0.004 | 0.011 | 0.003 | 0.001 |
| 4-2 | Average | 0.113 | −0.013 | −0.006 | 0.015 | −0.006 | 0.009 | 0.009 | 0.002 |
| | SD | 0.031 | 0.015 | 0.016 | 0.001 | 0.014 | 0.002 | 0.007 | 0.013 |
| | SEM | 0.018 | 0.009 | 0.009 | 0.001 | 0.008 | 0.001 | 0.004 | 0.008 |
| 16 | Average | 0.416 | 0.322 | 0.130 | −0.020 | −0.010 | 0.013 | 0.012 | −0.005 |
| | SD | 0.026 | 0.038 | 0.020 | 0.002 | 0.010 | 0.015 | 0.006 | 0.019 |
| | SEM | 0.015 | 0.022 | 0.012 | 0.001 | 0.006 | 0.009 | 0.003 | 0.011 |
| RC-1 | Average | 0.329 | 0.080 | −0.029 | −0.019 | −0.015 | −0.010 | −0.003 | −0.005 |
| | SD | 0.107 | 0.011 | 0.011 | 0.023 | 0.005 | 0.006 | 0.014 | 0.025 |
| | SEM | 0.062 | 0.006 | 0.006 | 0.013 | 0.003 | 0.004 | 0.008 | 0.014 |

E3-2:

| | | | | | | | | | |
|---|---|---|---|---|---|---|---|---|---|
| DCA | Average | 0.207 | 0.222 | 0.167 | 0.085 | 0.027 | −0.026 | −0.027 | −0.023 |
| | SD | 0.011 | 0.008 | 0.022 | 0.024 | 0.013 | 0.015 | 0.012 | 0.008 |
| | SEM | 0.006 | 0.004 | 0.013 | 0.014 | 0.007 | 0.008 | 0.007 | 0.004 |
| OCA | Average | 0.567 | 0.170 | 0.054 | −0.034 | −0.028 | −0.032 | −0.020 | −0.025 |
| | SD | 0.011 | 0.021 | 0.012 | 0.002 | 0.009 | 0.007 | 0.008 | 0.014 |
| | SEM | 0.006 | 0.012 | 0.007 | 0.001 | 0.005 | 0.004 | 0.005 | 0.008 |
| RC-2 | Average | 0.242 | 0.159 | 0.126 | 0.034 | 0.017 | −0.012 | −0.010 | −0.018 |
| | SD | 0.011 | 0.013 | 0.007 | 0.013 | 0.028 | 0.006 | 0.013 | 0.005 |
| | SEM | 0.007 | 0.007 | 0.004 | 0.007 | 0.016 | 0.004 | 0.008 | 0.003 |
| RC-3 | Average | 0.225 | 0.233 | 0.182 | 0.026 | −0.007 | −0.027 | −0.008 | −0.013 |
| | SD | 0.028 | 0.006 | 0.024 | 0.055 | 0.009 | 0.006 | 0.012 | 0.008 |
| | SEM | 0.016 | 0.003 | 0.014 | 0.032 | 0.005 | 0.003 | 0.007 | 0.005 |
| RC-5 | Average | 0.300 | 0.228 | 0.166 | 0.030 | −0.009 | −0.017 | −0.021 | −0.007 |
| | SD | 0.005 | 0.022 | 0.005 | 0.015 | 0.011 | 0.011 | 0.007 | 0.012 |
| | SEM | 0.003 | 0.013 | 0.003 | 0.009 | 0.006 | 0.006 | 0.004 | 0.007 |
| RC-6 | Average | 0.259 | 0.208 | 0.145 | 0.009 | −0.013 | −0.007 | −0.015 | −0.008 |
| | SD | 0.006 | 0.005 | 0.012 | 0.007 | 0.013 | 0.004 | 0.011 | 0.014 |
| | SEM | 0.004 | 0.003 | 0.007 | 0.004 | 0.008 | 0.002 | 0.006 | 0.008 |
| 1-4 | Average | 0.098 | 0.004 | −0.014 | −0.004 | −0.011 | −0.006 | −0.009 | −0.020 |
| | SD | 0.002 | 0.008 | 0.014 | 0.031 | 0.011 | 0.012 | 0.005 | 0.003 |
| | SEM | 0.001 | 0.004 | 0.008 | 0.018 | 0.007 | 0.007 | 0.003 | 0.001 |

E3-3:

| | | | | | | | | | |
|---|---|---|---|---|---|---|---|---|---|
| DCA | Average | 0.100 | 0.096 | 0.110 | 0.014 | 0.037 | −0.025 | −0.014 | −0.014 |
| | SD | 0.016 | 0.016 | 0.030 | 0.030 | 0.020 | 0.003 | 0.012 | 0.006 |
| | SEM | 0.009 | 0.009 | 0.017 | 0.017 | 0.011 | 0.002 | 0.007 | 0.004 |
| OCA | Average | 0.465 | 0.124 | 0.001 | −0.008 | −0.016 | −0.017 | −0.017 | −0.007 |
| | SD | 0.029 | 0.008 | 0.017 | 0.011 | 0.017 | 0.008 | 0.001 | 0.012 |
| | SEM | 0.017 | 0.005 | 0.010 | 0.006 | 0.010 | 0.005 | 0.001 | 0.007 |
| 1-6 | Average | 0.018 | −0.031 | −0.001 | −0.012 | −0.006 | −0.001 | 0.001 | −0.014 |
| | SD | 0.008 | 0.014 | 0.013 | 0.019 | 0.015 | 0.015 | 0.022 | 0.021 |
| | SEM | 0.005 | 0.008 | 0.008 | 0.011 | 0.009 | 0.009 | 0.012 | 0.012 |
| 17 | Average | 0.042 | 0.032 | −0.029 | −0.020 | −0.022 | −0.012 | −0.019 | −0.023 |
| | SD | 0.027 | 0.011 | 0.008 | 0.016 | 0.015 | 0.008 | 0.012 | 0.008 |
| | SEM | 0.016 | 0.006 | 0.004 | 0.009 | 0.009 | 0.005 | 0.007 | 0.005 |
| 1-2 | Average | 0.127 | 0.045 | 0.022 | −0.031 | −0.034 | −0.008 | −0.032 | 0.001 |
| | SD | 0.001 | 0.013 | 0.003 | 0.012 | 0.005 | 0.004 | 0.010 | 0.011 |
| | SEM | 0.001 | 0.007 | 0.002 | 0.007 | 0.003 | 0.003 | 0.006 | 0.007 |
| RC-7 | Average | 0.049 | 0.030 | −0.027 | −0.038 | −0.035 | 0.007 | −0.030 | −0.005 |
| | SD | 0.002 | 0.001 | 0.010 | 0.000 | 0.002 | 0.007 | 0.003 | 0.015 |
| | SEM | 0.001 | 0.000 | 0.006 | 0.000 | 0.001 | 0.004 | 0.002 | 0.009 |
| 84 | Average | 0.287 | 0.065 | 0.021 | −0.001 | 0.009 | −0.014 | 0.002 | −0.014 |
| | SD | 0.011 | 0.014 | 0.013 | 0.031 | 0.016 | 0.026 | 0.017 | 0.011 |
| | SEM | 0.006 | 0.008 | 0.008 | 0.018 | 0.009 | 0.015 | 0.010 | 0.006 |
| 2-6 | Average | 0.006 | −0.051 | −0.047 | −0.033 | −0.028 | −0.025 | −0.030 | −0.021 |
| | SD | 0.063 | 0.007 | 0.010 | 0.013 | 0.027 | 0.013 | 0.007 | 0.017 |
| | SEM | 0.036 | 0.004 | 0.006 | 0.007 | 0.016 | 0.008 | 0.004 | 0.010 |
| 3-3 | Average | 0.041 | 0.052 | −0.045 | −0.013 | −0.015 | −0.015 | −0.031 | −0.014 |
| | SD | 0.014 | 0.012 | 0.010 | 0.006 | 0.002 | 0.012 | 0.007 | 0.015 |
| | SEM | 0.008 | 0.007 | 0.006 | 0.003 | 0.001 | 0.007 | 0.004 | 0.009 |
| 3-2 | Average | 0.130 | 0.023 | −0.003 | −0.034 | 0.008 | −0.014 | −0.016 | −0.035 |
| | SD | 0.011 | 0.015 | 0.009 | 0.017 | 0.017 | 0.012 | 0.008 | 0.019 |
| | SEM | 0.006 | 0.009 | 0.005 | 0.010 | 0.010 | 0.007 | 0.005 | 0.011 |
| 1-7 | Average | 0.083 | 0.011 | −0.048 | −0.037 | −0.044 | −0.045 | −0.040 | −0.034 |
| | SD | 0.070 | 0.020 | 0.008 | 0.011 | 0.009 | 0.008 | 0.010 | 0.020 |
| | SEM | 0.041 | 0.012 | 0.004 | 0.006 | 0.005 | 0.005 | 0.006 | 0.012 |
| 3-1 | Average | 0.008 | 0.004 | −0.027 | −0.004 | −0.017 | 0.005 | 0.001 | −0.001 |
| | SD | 0.007 | 0.001 | 0.011 | 0.015 | 0.018 | 0.018 | 0.009 | 0.015 |
| | SEM | 0.004 | 0.001 | 0.006 | 0.009 | 0.010 | 0.010 | 0.005 | 0.009 |
| 2-5 | Average | 0.006 | 0.009 | −0.031 | −0.011 | 0.001 | 0.017 | −0.015 | −0.015 |
| | SD | 0.002 | 0.008 | 0.005 | 0.002 | 0.024 | 0.007 | 0.006 | 0.011 |
| | SEM | 0.001 | 0.005 | 0.003 | 0.001 | 0.014 | 0.004 | 0.004 | 0.006 |
| 21 | Average | 0.099 | −0.022 | 0.024 | −0.024 | 0.007 | −0.003 | 0.010 | −0.010 |
| | SD | 0.004 | 0.007 | 0.020 | 0.012 | 0.023 | 0.015 | 0.012 | 0.025 |
| | SEM | 0.002 | 0.004 | 0.011 | 0.007 | 0.013 | 0.009 | 0.007 | 0.014 |
| 4-3 | Average | −0.005 | −0.003 | −0.029 | −0.018 | −0.030 | −0.016 | −0.026 | −0.025 |
| | SD | 0.026 | 0.029 | 0.007 | 0.013 | 0.004 | 0.014 | 0.007 | 0.011 |
| | SEM | 0.015 | 0.017 | 0.004 | 0.008 | 0.003 | 0.008 | 0.004 | 0.006 |
| 1-3 | Average | 0.109 | 0.114 | 0.047 | −0.026 | −0.008 | 0.018 | −0.003 | 0.008 |
| | SD | 0.043 | 0.009 | 0.005 | 0.002 | 0.016 | 0.011 | 0.012 | 0.015 |
| | SEM | 0.025 | 0.005 | 0.003 | 0.001 | 0.009 | 0.007 | 0.007 | 0.009 |
| 9-7 | Average | −0.001 | 0.022 | 0.009 | −0.023 | −0.005 | 0.019 | −0.005 | 0.003 |
| | SD | 0.008 | 0.009 | 0.006 | 0.005 | 0.022 | 0.018 | 0.002 | 0.016 |
| | SEM | 0.005 | 0.005 | 0.003 | 0.003 | 0.013 | 0.011 | 0.001 | 0.009 |
| RC-4 | Average | 0.107 | 0.143 | 0.106 | −0.012 | −0.012 | 0.024 | 0.015 | −0.007 |
| | SD | 0.009 | 0.005 | 0.010 | 0.016 | 0.016 | 0.015 | 0.005 | 0.027 |
| | SEM | 0.005 | 0.003 | 0.006 | 0.009 | 0.009 | 0.009 | 0.003 | 0.015 |

E3-4:

| | | | | | | | | | |
|---|---|---|---|---|---|---|---|---|---|
| DCA | Average | 1.101 | 0.998 | 1.108 | 0.548 | −0.028 | −0.049 | −0.051 | −0.045 |
| | SD | 0.186 | 0.253 | 0.235 | 0.040 | 0.007 | 0.009 | 0.017 | 0.003 |
| | SEM | 0.107 | 0.146 | 0.136 | 0.023 | 0.004 | 0.005 | 0.010 | 0.002 |
| CDCA | Average | 1.098 | 0.586 | 0.082 | −0.017 | −0.057 | −0.044 | −0.049 | −0.070 |
| | SD | 0.054 | 0.087 | 0.067 | 0.083 | 0.004 | 0.029 | 0.009 | 0.025 |
| | SEM | 0.031 | 0.050 | 0.039 | 0.048 | 0.002 | 0.017 | 0.005 | 0.014 |
| OCA | Average | 1.372 | 0.683 | 0.066 | −0.068 | −0.048 | −0.040 | −0.056 | −0.079 |
| | SD | 0.072 | 0.202 | 0.012 | 0.006 | 0.005 | 0.005 | 0.037 | 0.042 |
| | SEM | 0.041 | 0.117 | 0.007 | 0.003 | 0.003 | 0.003 | 0.021 | 0.024 |
| 10 | Average | 0.019 | −0.031 | −0.037 | −0.032 | −0.064 | −0.043 | −0.045 | −0.048 |
| | SD | 0.007 | 0.011 | 0.019 | 0.002 | 0.043 | 0.012 | 0.026 | 0.007 |
| | SEM | 0.004 | 0.006 | 0.011 | 0.001 | 0.025 | 0.007 | 0.015 | 0.004 |
| 5-1 | Average | 0.068 | −0.017 | −0.047 | −0.038 | −0.065 | −0.039 | −0.041 | −0.043 |
| | SD | 0.011 | 0.009 | 0.018 | 0.080 | 0.012 | 0.007 | 0.001 | 0.014 |
| | SEM | 0.006 | 0.005 | 0.010 | 0.046 | 0.007 | 0.004 | 0.001 | 0.008 |
| 1-5 | Average | 0.035 | −0.027 | −0.045 | −0.034 | −0.041 | −0.027 | −0.049 | −0.084 |
| | SD | 0.004 | 0.010 | 0.047 | 0.012 | 0.008 | 0.006 | 0.053 | 0.022 |
| | SEM | 0.002 | 0.006 | 0.027 | 0.007 | 0.004 | 0.004 | 0.031 | 0.012 |
| 26 | Average | −0.070 | −0.059 | −0.090 | −0.062 | −0.067 | −0.072 | −0.090 | −0.084 |
| | SD | 0.019 | 0.003 | 0.037 | 0.009 | 0.007 | 0.006 | 0.043 | 0.009 |
| | SEM | 0.011 | 0.002 | 0.021 | 0.005 | 0.004 | 0.003 | 0.025 | 0.005 |
| 2-1 | Average | 0.071 | −0.014 | −0.045 | −0.072 | −0.095 | −0.110 | −0.072 | −0.091 |
| | SD | 0.014 | 0.005 | 0.017 | 0.018 | 0.013 | 0.006 | 0.029 | 0.045 |
| | SEM | 0.008 | 0.003 | 0.010 | 0.010 | 0.008 | 0.004 | 0.016 | 0.026 |
| 9-7 | Average | 0.039 | 0.037 | −0.002 | −0.073 | −0.048 | −0.037 | −0.052 | −0.034 |
| | SD | 0.037 | 0.003 | 0.002 | 0.020 | 0.022 | 0.016 | 0.033 | 0.010 |
| | SEM | 0.022 | 0.002 | 0.001 | 0.011 | 0.013 | 0.009 | 0.019 | 0.006 |
| RC-4 | Average | 1.229 | 0.740 | 0.252 | −0.067 | −0.058 | −0.040 | −0.037 | −0.021 |
| | SD | 0.040 | 0.130 | 0.051 | 0.025 | 0.032 | 0.021 | 0.005 | 0.002 |
| | SEM | 0.023 | 0.075 | 0.030 | 0.014 | 0.018 | 0.012 | 0.003 | 0.001 |
| 9-2 | Average | 0.655 | 0.162 | 0.011 | −0.068 | −0.030 | −0.029 | −0.035 | −0.072 |
| | SD | 0.064 | 0.087 | 0.024 | 0.015 | 0.020 | 0.006 | 0.035 | 0.025 |
| | SEM | 0.037 | 0.050 | 0.014 | 0.009 | 0.011 | 0.004 | 0.020 | 0.014 |
| 2-2 | Average | 0.006 | −0.080 | −0.068 | −0.039 | −0.069 | −0.034 | −0.062 | −0.057 |
| | SD | 0.018 | 0.012 | 0.025 | 0.019 | 0.036 | 0.061 | 0.023 | 0.035 |
| | SEM | 0.010 | 0.007 | 0.015 | 0.011 | 0.021 | 0.035 | 0.013 | 0.020 |

E3-5:

| | | | | | | | | | |
|---|---|---|---|---|---|---|---|---|---|
| DCA | Average | 1.383 | 1.126 | 0.905 | 0.451 | 0.053 | −0.056 | −0.052 | −0.047 |
| | SD | 0.077 | 0.056 | 0.212 | 0.106 | 0.076 | 0.021 | 0.025 | 0.022 |
| | SEM | 0.044 | 0.032 | 0.122 | 0.061 | 0.044 | 0.012 | 0.015 | 0.012 |
| OCA | Average | 1.188 | 0.237 | −0.048 | −0.068 | −0.069 | −0.069 | −0.051 | −0.049 |
| | SD | 0.001 | 0.086 | 0.019 | 0.006 | 0.003 | 0.002 | 0.005 | 0.002 |
| | SEM | 0.001 | 0.050 | 0.011 | 0.004 | 0.002 | 0.001 | 0.003 | 0.001 |
| 2-4 | Average | 0.008 | −0.025 | −0.026 | −0.018 | −0.037 | −0.025 | −0.038 | −0.037 |
| | SD | 0.005 | 0.010 | 0.010 | 0.013 | 0.015 | 0.011 | 0.027 | 0.024 |
| | SEM | 0.003 | 0.006 | 0.006 | 0.007 | 0.009 | 0.007 | 0.016 | 0.014 |
| 2-5 | Average | 0.014 | −0.013 | −0.038 | −0.049 | −0.064 | −0.059 | −0.066 | −0.058 |
| | SD | 0.010 | 0.012 | 0.030 | 0.014 | 0.017 | 0.018 | 0.021 | 0.018 |
| | SEM | 0.006 | 0.007 | 0.017 | 0.008 | 0.010 | 0.010 | 0.012 | 0.011 |
| 4-3 | Average | 0.035 | −0.006 | −0.066 | −0.082 | −0.056 | −0.067 | −0.042 | −0.064 |
| | SD | 0.006 | 0.001 | 0.008 | 0.008 | 0.005 | 0.009 | 0.013 | 0.024 |
| | SEM | 0.003 | 0.001 | 0.005 | 0.005 | 0.003 | 0.005 | 0.007 | 0.014 |
| 21 | Average | 0.046 | 0.001 | −0.026 | −0.026 | −0.059 | −0.049 | −0.034 | −0.048 |
| | SD | 0.021 | 0.008 | 0.007 | 0.016 | 0.022 | 0.023 | 0.016 | 0.027 |
| | SEM | 0.012 | 0.004 | 0.004 | 0.009 | 0.013 | 0.013 | 0.009 | 0.016 |
| 2-6 | Average | 0.503 | 0.039 | −0.047 | −0.056 | −0.044 | −0.045 | −0.025 | −0.040 |
| | SD | 0.093 | 0.036 | 0.009 | 0.019 | 0.015 | 0.016 | 0.008 | 0.008 |
| | SEM | 0.054 | 0.020 | 0.005 | 0.011 | 0.009 | 0.009 | 0.004 | 0.004 |
| 1-2 | Average | 1.111 | 0.622 | 0.027 | −0.088 | −0.073 | −0.064 | −0.054 | −0.062 |
| | SD | 0.055 | 0.039 | 0.054 | 0.011 | 0.020 | 0.027 | 0.000 | 0.003 |
| | SEM | 0.032 | 0.022 | 0.031 | 0.007 | 0.012 | 0.015 | 0.000 | 0.002 |
| 17 | Average | 0.036 | −0.001 | −0.032 | −0.024 | −0.066 | −0.035 | −0.053 | −0.056 |
| | SD | 0.027 | 0.018 | 0.019 | 0.009 | 0.025 | 0.003 | 0.032 | 0.036 |
| | SEM | 0.016 | 0.011 | 0.011 | 0.005 | 0.014 | 0.002 | 0.018 | 0.021 |

E3-6-1:

| | | | | | | | | | |
|---|---|---|---|---|---|---|---|---|---|
| DCA | Average | 0.730 | 0.820 | 0.687 | 0.340 | 0.047 | 0.017 | 0.017 | 0.017 |
| | SD | 0.139 | 0.026 | 0.012 | 0.075 | 0.015 | 0.012 | 0.006 | 0.006 |
| | SEM | 0.080 | 0.015 | 0.007 | 0.044 | 0.009 | 0.007 | 0.003 | 0.003 |
| OCA | Average | | | | | | | | |
| | SD | 0.680 | 0.330 | 0.027 | 0.017 | 0.027 | 0.013 | 0.017 | 0.010 |
| | SEM | 0.035 | 0.061 | 0.015 | 0.006 | 0.006 | 0.006 | 0.006 | 0.000 |
| 5-1 | Average | 0.020 | 0.035 | 0.009 | 0.003 | 0.003 | 0.003 | 0.003 | 0.000 |
| | SD | | | | | | | | |
| | SEM | 0.080 | 0.037 | 0.043 | 0.040 | 0.037 | 0.017 | 0.017 | 0.030 |

E3-6-2:

| | | | | | | | |
|---|---|---|---|---|---|---|---|
| 5-2 | Average | 0.087 | 0.030 | 0.023 | 0.027 | 0.023 | 0.020 |
| | SD | 0.035 | 0.010 | 0.015 | 0.012 | 0.006 | 0.010 |
| | SEM | 0.020 | 0.006 | 0.009 | 0.007 | 0.003 | 0.006 |
| 5-4 | Average | 0.097 | 0.017 | 0.020 | 0.020 | 0.013 | 0.017 |
| | SD | 0.012 | 0.012 | 0.010 | 0.000 | 0.006 | 0.006 |
| | SEM | 0.007 | 0.007 | 0.006 | 0.000 | 0.003 | 0.003 |
| 5-5 | Average | 0.083 | 0.033 | 0.017 | 0.020 | 0.027 | 0.060 |
| | SD | 0.006 | 0.015 | 0.006 | 0.000 | 0.021 | 0.010 |
| | SEM | 0.003 | 0.009 | 0.003 | 0.000 | 0.012 | 0.006 |
| 5-6 | Average | 0.077 | 0.033 | 0.010 | 0.017 | 0.013 | 0.010 |
| | SD | 0.006 | 0.006 | 0.000 | 0.006 | 0.006 | 0.000 |
| | SEM | 0.003 | 0.003 | 0.000 | 0.003 | 0.003 | 0.000 |
| 5-7 | Average | 0.070 | 0.027 | 0.030 | 0.020 | 0.020 | 0.017 |
| | SD | 0.017 | 0.021 | 0.017 | 0.010 | 0.000 | 0.006 |
| | SEM | 0.010 | 0.012 | 0.010 | 0.006 | 0.000 | 0.003 |
| 5-8 | Average | 0.073 | 0.037 | 0.023 | 0.017 | 0.020 | 0.023 |
| | SD | 0.015 | 0.006 | 0.006 | 0.006 | 0.010 | 0.006 |
| | SEM | 0.009 | 0.003 | 0.003 | 0.003 | 0.006 | 0.003 |
| 5-10 | Average | 0.090 | 0.043 | 0.013 | 0.017 | 0.020 | 0.030 |
| | SD | 0.010 | 0.006 | 0.006 | 0.006 | 0.017 | 0.000 |
| | SEM | 0.006 | 0.003 | 0.003 | 0.003 | 0.010 | 0.000 |
| 5-11 | Average | 0.057 | 0.030 | 0.033 | 0.013 | 0.023 | 0.067 |
| | SD | 0.006 | 0.010 | 0.021 | 0.006 | 0.015 | 0.035 |
| | SEM | 0.003 | 0.006 | 0.012 | 0.003 | 0.009 | 0.020 |
| 5-12 | Average | 0.057 | 0.013 | 0.023 | 0.013 | 0.013 | 0.013 |
| | SD | 0.006 | 0.006 | 0.015 | 0.006 | 0.006 | 0.006 |
| | SEM | 0.003 | 0.003 | 0.009 | 0.003 | 0.003 | 0.003 |
| 5-13 | Average | 0.060 | 0.020 | 0.030 | 0.017 | 0.017 | 0.027 |
| | SD | 0.020 | 0.017 | 0.026 | 0.006 | 0.006 | 0.012 |
| | SEM | 0.012 | 0.010 | 0.015 | 0.003 | 0.003 | 0.007 |
| 5-14 | Average | 0.070 | 0.023 | 0.017 | 0.020 | 0.017 | 0.017 |
| | SD | 0.010 | 0.006 | 0.006 | 0.010 | 0.006 | 0.006 |
| | SEM | 0.006 | 0.003 | 0.003 | 0.006 | 0.003 | 0.003 |
| 5-15 | Average | 0.087 | 0.030 | 0.013 | 0.027 | 0.010 | 0.010 |
| | SD | 0.012 | 0.010 | 0.006 | 0.015 | 0.000 | 0.000 |
| | SEM | 0.007 | 0.006 | 0.003 | 0.009 | 0.000 | 0.000 |

E3-7:

| | OCA | | 5-23 | | 8-4 | | 12-2 | | 13-2 | |
|---|---|---|---|---|---|---|---|---|---|---|
| Conc. (Log[M]) | Ave. | SEM | Ave. | SEM | Ave. | SEM | Ave. | SEM | Ave. | SEM |
| −4 | 1.108 | 0.270 | −0.027 | 0.023 | −0.041 | 0.023 | 0.000 | 0.062 | −0.054 | 0.000 |
| −4.5 | 0.351 | 0.041 | −0.027 | 0.023 | −0.027 | 0.023 | −0.041 | 0.023 | −0.054 | 0.000 |
| −5 | −0.041 | 0.023 | −0.014 | 0.000 | −0.014 | 0.041 | −0.041 | 0.023 | −0.027 | 0.023 |
| −5.5 | −0.014 | 0.041 | −0.054 | 0.000 | −0.014 | 0.000 | −0.054 | 0.041 | −0.027 | 0.023 |
| −6 | −0.027 | 0.023 | −0.041 | 0.023 | 0.000 | 0.023 | −0.041 | 0.023 | −0.027 | 0.023 |
| −7 | −0.014 | 0.000 | 0.000 | 0.023 | 0.000 | 0.023 | −0.027 | 0.023 | 0.000 | 0.023 |

E3-8-1:

| | |
|---|---|
| Compound | Compound |
| OCA | DCA |
| Activation (%) | Activation (%) |

| Conc. (nM) | n = 1 | n = 2 | Conc. (nM) | n = 1 | n = 2 |
|---|---|---|---|---|---|
| 2000000 | 191.4 | 158.9 | 100000 | 96.4 | 107.2 |
| 1000000 | 151.8 | 149.6 | 33333 | 95.1 | 98.5 |
| 500000 | 138.8 | 110.9 | 11111 | 73.8 | 56.4 |
| 250000 | 104.4 | 100.4 | 3704 | 37.5 | 30.1 |
| 125000 | 100.4 | 85.8 | 1235 | 14.9 | 6.58 |
| 62500 | 65.7 | 49.9 | 412 | 3.48 | 0.70 |
| 31250 | 13.7 | 25.8 | 137 | 1.01 | 0.39 |
| 15625 | 6.58 | 9.06 | 46 | 2.24 | 0.70 |
| 7813 | 3.48 | 4.10 | 15 | 1.01 | 0.39 |
| 3906 | 3.48 | 4.41 | 5 | 1.93 | 0.08 |

E3-8-1:

| | Compound | | | | | | | | | |
|---|---|---|---|---|---|---|---|---|---|---|
| | 5-26 | | 20-3 | | 18-2 | | 18-1 | | 18-3 | |
| | Activation (%) | | Activation (%) | | Activation (%) | | Activation (%) | | Activation (%) | |
| Conc. (nM) | n = 1 | n = 2 | n = 1 | n = 2 | n = 1 | n = 2 | n = 1 | n = 2 | n = 1 | n = 2 |
| 30000 | 2.86 | 2.86 | 1.63 | 2.24 | 4.10 | 3.48 | 2.24 | 2.24 | 1.63 | 0.08 |
| 10000 | 3.17 | 4.10 | 2.55 | 3.17 | 3.48 | 2.24 | 2.86 | 3.79 | 2.24 | 2.86 |
| 3333 | 3.17 | 3.79 | 2.55 | 3.17 | 3.17 | 1.93 | 1.93 | 2.55 | 3.17 | 2.24 |
| 1111 | 3.17 | 3.48 | 3.79 | 3.48 | 2.24 | 2.55 | 2.86 | 2.86 | 2.55 | 2.24 |
| 370 | 2.24 | 4.10 | 3.17 | 3.17 | 3.48 | 3.48 | 3.17 | 3.48 | 2.24 | 3.48 |
| 123 | 3.48 | 3.79 | 3.17 | 3.79 | 3.48 | 2.86 | 2.55 | 3.17 | 2.86 | 2.86 |
| 41 | 3.17 | 3.17 | 3.48 | 3.48 | 3.17 | 1.93 | 2.55 | 3.48 | 2.86 | 2.55 |
| 14 | 2.86 | 3.17 | 3.48 | 3.79 | 3.79 | 3.79 | 2.86 | 3.17 | 2.86 | 2.55 |
| 4.6 | 3.79 | 3.17 | 3.48 | 2.86 | 2.86 | 3.17 | 3.17 | 2.86 | 3.17 | 2.86 |
| 1.5 | 2.55 | 3.48 | 2.55 | 3.17 | 3.17 | 3.48 | 2.86 | 3.17 | 2.24 | 2.55 |

E3-9:

| Compound | | | |
|---|---|---|---|
| DCA-1 | DCA-2 | DCA-3 | DCA-4 |

| Conc. (Log[M]) | Average | SEM | Average | SEM | Average | SEM | Average | SEM |
|---|---|---|---|---|---|---|---|---|
| −4.0 | 0.965 | 0.035 | 0.980 | 0.026 | 0.945 | 0.024 | 1.001 | 0.002 |
| −4.5 | 0.754 | 0.016 | 0.901 | 0.061 | 0.805 | 0.014 | 0.861 | 0.073 |
| −5.0 | 0.522 | 0.019 | 0.632 | 0.042 | 0.517 | 0.002 | 0.692 | 0.056 |
| −5.4 | 0.168 | 0.007 | 0.193 | 0.011 | 0.153 | 0.014 | 0.250 | 0.015 |
| −5.9 | 0.051 | 0.007 | 0.063 | 0.009 | 0.034 | 0.002 | 0.072 | 0.007 |
| −6.4 | 0.001 | 0.000 | 0.008 | 0.000 | 0.008 | 0.000 | 0.010 | 0.000 |
| −6.9 | 0.013 | 0.002 | 0.014 | 0.002 | 0.010 | 0.002 | 0.014 | 0.000 |
| −7.3 | 0.006 | 0.005 | 0.008 | 0.004 | 0.008 | 0.000 | 0.012 | 0.002 |
| −7.8 | 0.016 | 0.005 | 0.004 | 0.004 | 0.003 | 0.000 | 0.010 | 0.000 |

Compound

| | | | |
|---|---|---|---|
| OCA-1 | OCA-2 | OCA-3 | OCA-4 |

| Conc. (Log[M]) | Average | SEM | Average | SEM | Average | SEM | Average | SEM |
|---|---|---|---|---|---|---|---|---|
| −3.7 | 2.385 | 0.016 | 1.685 | 0.002 | 2.625 | 0.150 | 1.629 | 0.047 |
| −4.0 | 1.277 | 0.089 | 0.980 | 0.004 | 1.367 | 0.041 | 1.012 | 0.100 |
| −4.3 | 0.461 | 0.061 | 0.348 | 0.011 | 0.493 | 0.012 | 0.374 | 0.025 |
| −4.6 | 0.109 | 0.028 | 0.087 | 0.004 | 0.102 | 0.007 | 0.107 | 0.005 |
| −4.9 | 0.020 | 0.000 | 0.031 | 0.008 | 0.025 | 0.002 | 0.032 | 0.004 |
| −5.2 | 0.011 | 0.009 | 0.014 | 0.002 | 0.013 | 0.000 | 0.021 | 0.000 |
| −5.5 | 0.018 | 0.002 | 0.008 | 0.000 | 0.008 | 0.005 | 0.010 | 0.000 |
| −5.8 | 0.011 | 0.009 | 0.019 | 0.004 | 0.020 | 0.002 | 0.025 | 0.004 |
| −6.1 | 0.018 | 0.002 | 0.012 | 0.000 | 0.015 | 0.002 | 0.021 | 0.004 |
| −6.4 | 0.016 | 0.009 | 0.014 | 0.002 | 0.020 | 0.002 | 0.019 | 0.002 |

Compound

| | | | |
|---|---|---|---|
| 5-25 | 6-2 | 6-3 | 6-4 |

| Conc. (Log[M]) | Average | SEM | Average | SEM | Average | SEM | Average | SEM |
|---|---|---|---|---|---|---|---|---|
| −4.0 | 0.393 | 0.035 | 0.030 | 0.000 | 0.016 | 0.005 | 0.027 | 0.002 |
| −4.5 | 0.016 | 0.014 | 0.023 | 0.002 | 0.008 | 0.002 | 0.011 | 0.000 |
| −5.0 | 0.001 | 0.005 | 0.013 | 0.002 | 0.020 | 0.000 | 0.011 | 0.000 |
| −5.4 | 0.016 | 0.000 | 0.020 | 0.000 | 0.016 | 0.005 | 0.018 | 0.007 |
| −5.9 | 0.011 | 0.005 | 0.016 | 0.000 | 0.018 | 0.002 | 0.016 | 0.000 |
| −6.4 | 0.011 | 0.000 | 0.016 | 0.005 | 0.018 | 0.002 | 0.011 | 0.000 |
| −6.9 | 0.008 | 0.002 | 0.016 | 0.009 | 0.011 | 0.000 | 0.018 | 0.002 |
| −7.3 | 0.013 | 0.002 | 0.018 | 0.002 | 0.018 | 0.002 | 0.018 | 0.002 |
| −7.8 | 0.013 | 0.002 | 0.011 | 0.000 | 0.020 | 0.000 | 0.020 | 0.000 |
| −8.3 | 0.018 | 0.002 | 0.018 | 0.002 | 0.013 | 0.002 | 0.018 | 0.002 |

Compound

| | | | |
|---|---|---|---|
| 6-5 | 7-1 | 53 | 8-2 |

| Conc. (Log[M]) | Average | SEM | Average | SEM | Average | SEM | Average | SEM |
|---|---|---|---|---|---|---|---|---|
| −4.0 | 0.025 | 0.005 | 0.020 | 0.000 | 0.030 | 0.005 | 0.020 | 0.005 |
| −4.5 | 0.020 | 0.005 | 0.018 | 0.002 | 0.016 | 0.000 | 0.018 | 0.007 |
| −5.0 | 0.018 | 0.007 | 0.013 | 0.002 | 0.013 | 0.002 | 0.018 | 0.002 |
| −5.4 | 0.020 | 0.000 | 0.013 | 0.002 | 0.020 | 0.000 | 0.016 | 0.005 |
| −5.9 | 0.018 | 0.007 | 0.011 | 0.000 | 0.011 | 0.000 | 0.018 | 0.002 |
| −6.4 | 0.016 | 0.005 | 0.013 | 0.002 | 0.013 | 0.002 | 0.011 | 0.000 |
| −6.9 | 0.018 | 0.002 | 0.004 | 0.002 | 0.013 | 0.002 | 0.018 | 0.002 |
| −7.3 | 0.016 | 0.000 | 0.008 | 0.002 | 0.013 | 0.002 | 0.008 | 0.002 |
| −7.8 | 0.020 | 0.000 | 0.006 | 0.000 | 0.008 | 0.002 | 0.011 | 0.000 |
| −8.3 | 0.018 | 0.002 | 0.011 | 0.000 | 0.011 | 0.005 | 0.006 | 0.005 |

Compound

| | | | |
|---|---|---|---|
| 8-3 | 8-5 | 9-1 | 9-2 |

| Conc. (Log[M]) | Average | SEM | Average | SEM | Average | SEM | Average | SEM |
|---|---|---|---|---|---|---|---|---|
| −4.0 | 0.023 | 0.002 | 0.030 | 0.005 | 0.019 | 0.004 | 0.035 | 0.008 |
| −4.5 | 0.016 | 0.000 | 0.016 | 0.000 | 0.021 | 0.002 | 0.001 | 0.004 |
| −5.0 | 0.016 | 0.000 | 0.018 | 0.002 | 0.012 | 0.004 | 0.002 | 0.002 |
| −5.4 | 0.023 | 0.002 | 0.018 | 0.002 | 0.018 | 0.002 | 0.019 | 0.004 |
| −5.9 | 0.025 | 0.005 | 0.018 | 0.002 | 0.014 | 0.006 | 0.018 | 0.002 |
| −6.4 | 0.013 | 0.002 | 0.016 | 0.000 | 0.019 | 0.000 | 0.018 | 0.002 |
| −6.9 | 0.013 | 0.002 | 0.020 | 0.000 | 0.016 | 0.004 | 0.014 | 0.006 |
| −7.3 | 0.013 | 0.002 | 0.016 | 0.005 | 0.016 | 0.000 | 0.019 | 0.004 |
| −7.8 | 0.011 | 0.005 | 0.011 | 0.005 | 0.012 | 0.000 | 0.016 | 0.000 |
| −8.3 | 0.018 | 0.002 | 0.011 | 0.000 | 0.018 | 0.002 | 0.019 | 0.000 |

Compound

| | | | |
|---|---|---|---|
| 9-3 | 9-4 | 9-5 | 9-6 |

| Conc. (Log[M]) | Average | SEM | Average | SEM | Average | SEM | Average | SEM |
|---|---|---|---|---|---|---|---|---|
| −4.0 | 0.006 | 0.006 | −0.003 | 0.000 | 0.014 | 0.002 | 0.021 | 0.002 |
| −4.5 | 0.004 | 0.000 | 0.004 | 0.004 | 0.016 | 0.004 | 0.014 | 0.002 |
| −5.0 | 0.018 | 0.002 | 0.014 | 0.002 | 0.023 | 0.000 | 0.016 | 0.000 |
| −5.4 | 0.019 | 0.004 | 0.019 | 0.000 | 0.023 | 0.004 | 0.010 | 0.002 |
| −5.9 | 0.019 | 0.004 | 0.019 | 0.004 | 0.018 | 0.006 | 0.019 | 0.000 |
| −6.4 | 0.016 | 0.004 | 0.018 | 0.002 | 0.019 | 0.000 | 0.018 | 0.002 |
| −6.9 | 0.014 | 0.006 | 0.019 | 0.000 | 0.021 | 0.002 | 0.016 | 0.000 |
| −7.3 | 0.018 | 0.002 | 0.021 | 0.002 | 0.019 | 0.000 | 0.014 | 0.002 |
| −7.8 | 0.016 | 0.000 | 0.018 | 0.002 | 0.021 | 0.002 | 0.012 | 0.000 |
| −8.3 | 0.016 | 0.004 | 0.019 | 0.000 | 0.021 | 0.002 | 0.016 | 0.000 |

Compound

| | | | |
|---|---|---|---|
| 55 | 10-1 | 10-2 | 10-3 |

| Conc. (Log[M]) | Average | SEM | Average | SEM | Average | SEM | Average | SEM |
|---|---|---|---|---|---|---|---|---|
| −4.0 | 0.006 | 0.002 | 0.025 | 0.006 | 0.006 | 0.002 | 0.010 | 0.002 |
| −4.5 | 0.004 | 0.000 | 0.016 | 0.000 | 0.004 | 0.004 | 0.004 | 0.004 |
| −5.0 | 0.018 | 0.002 | 0.021 | 0.002 | 0.010 | 0.002 | 0.014 | 0.002 |
| −5.4 | 0.023 | 0.000 | 0.018 | 0.002 | 0.018 | 0.002 | 0.016 | 0.004 |
| −5.9 | 0.019 | 0.000 | 0.019 | 0.004 | 0.018 | 0.002 | 0.023 | 0.000 |
| −6.4 | 0.019 | 0.004 | 0.016 | 0.004 | 0.019 | 0.000 | 0.018 | 0.002 |
| −6.9 | 0.016 | 0.000 | 0.018 | 0.002 | 0.021 | 0.002 | 0.023 | 0.000 |
| −7.3 | 0.019 | 0.000 | 0.016 | 0.004 | 0.019 | 0.000 | 0.021 | 0.002 |
| −7.8 | 0.016 | 0.004 | 0.008 | 0.000 | 0.018 | 0.002 | 0.012 | 0.000 |
| −8.3 | 0.018 | 0.002 | 0.010 | 0.002 | 0.018 | 0.002 | 0.018 | 0.002 |

Compound

| | | | |
|---|---|---|---|
| 10-4 | 11-2 | 11-3 | 11-4 |

| Conc. (Log[M]) | Average | SEM | Average | SEM | Average | SEM | Average | SEM |
|---|---|---|---|---|---|---|---|---|
| −4.0 | −0.004 | 0.002 | 0.030 | 0.002 | 0.003 | 0.005 | 0.013 | 0.005 |
| −4.5 | 0.001 | 0.002 | 0.020 | 0.002 | 0.010 | 0.002 | 0.013 | 0.005 |
| −5.0 | 0.008 | 0.005 | 0.010 | 0.002 | 0.015 | 0.002 | 0.020 | 0.002 |
| −5.4 | 0.022 | 0.000 | 0.020 | 0.002 | 0.015 | 0.002 | 0.015 | 0.002 |
| −5.9 | 0.013 | 0.000 | 0.020 | 0.002 | 0.015 | 0.002 | 0.015 | 0.002 |
| −6.4 | 0.015 | 0.002 | 0.013 | 0.000 | 0.008 | 0.000 | 0.013 | 0.005 |
| −6.9 | 0.013 | 0.000 | 0.013 | 0.005 | 0.010 | 0.002 | 0.010 | 0.002 |
| −7.3 | 0.010 | 0.002 | 0.013 | 0.000 | 0.015 | 0.002 | 0.015 | 0.002 |
| −7.8 | 0.010 | 0.002 | 0.013 | 0.000 | 0.018 | 0.000 | 0.015 | 0.002 |
| −8.3 | 0.020 | 0.002 | 0.025 | 0.002 | 0.051 | 0.034 | 0.022 | 0.005 |

Compound

| | | | |
|---|---|---|---|
| 12-1 | 13-1 | 14-1 | 14-2 |

| Conc. (Log[M]) | Average | SEM | Average | SEM | Average | SEM | Average | SEM |
|---|---|---|---|---|---|---|---|---|
| −4.0 | 0.196 | 0.048 | 0.104 | 0.005 | 0.030 | 0.002 | 0.090 | 0.005 |
| −4.5 | −0.004 | 0.002 | −0.009 | 0.002 | 0.013 | 0.000 | 0.027 | 0.000 |
| −5.0 | 0.010 | 0.002 | 0.013 | 0.005 | 0.015 | 0.002 | 0.027 | 0.000 |
| −5.4 | 0.018 | 0.000 | 0.013 | 0.000 | 0.020 | 0.002 | 0.030 | 0.002 |
| −5.9 | 0.015 | 0.002 | 0.018 | 0.000 | 0.015 | 0.002 | 0.020 | 0.002 |
| −6.4 | 0.013 | 0.005 | 0.015 | 0.002 | 0.018 | 0.005 | 0.013 | 0.000 |
| −6.9 | 0.018 | 0.000 | 0.018 | 0.000 | 0.013 | 0.000 | 0.013 | 0.000 |
| −7.3 | 0.015 | 0.002 | 0.013 | 0.000 | 0.015 | 0.002 | 0.013 | 0.000 |
| −7.8 | 0.018 | 0.000 | 0.010 | 0.002 | 0.013 | 0.000 | 0.010 | 0.002 |
| −8.3 | 0.027 | 0.014 | 0.013 | 0.005 | 0.010 | 0.002 | 0.010 | 0.002 |

Compound

| | | | |
|---|---|---|---|
| 14-3 | 14-4 | 5-29 | 5-30 |

| Conc. (Log[M]) | Average | SEM | Average | SEM | Average | SEM | Average | SEM |
|---|---|---|---|---|---|---|---|---|
| −4.0 | 0.071 | 0.034 | 0.061 | 0.014 | 0.014 | 0.004 | 0.014 | 0.000 |
| −4.5 | 0.030 | 0.007 | 0.020 | 0.007 | 0.014 | 0.000 | 0.018 | 0.004 |
| −5.0 | 0.015 | 0.002 | 0.013 | 0.000 | 0.010 | 0.004 | 0.014 | 0.004 |
| −5.4 | 0.020 | 0.002 | 0.018 | 0.000 | 0.018 | 0.011 | 0.027 | 0.009 |
| −5.9 | 0.018 | 0.000 | 0.013 | 0.000 | 0.016 | 0.002 | 0.018 | 0.004 |
| −6.4 | 0.013 | 0.000 | 0.020 | 0.002 | 0.018 | 0.004 | 0.023 | 0.002 |
| −6.9 | 0.015 | 0.002 | 0.015 | 0.002 | 0.018 | 0.002 | 0.021 | 0.004 |
| −7.3 | 0.015 | 0.002 | 0.015 | 0.002 | 0.016 | 0.000 | 0.025 | 0.000 |
| −7.8 | 0.008 | 0.000 | 0.008 | 0.000 | 0.020 | 0.000 | 0.018 | 0.004 |
| −8.3 | 0.008 | 0.000 | 0.015 | 0.002 | 0.018 | 0.002 | 0.028 | 0.000 |

Compound

| | | | |
|---|---|---|---|
| 20-1 | 20-2 | 18-1 | 18-3 |

| Conc. (Log[M]) | Average | SEM | Average | SEM | Average | SEM | Average | SEM |
|---|---|---|---|---|---|---|---|---|
| −4.0 | 0.018 | 0.000 | 0.025 | 0.004 | 0.168 | 0.009 | 0.519 | 0.062 |
| −4.5 | 0.016 | 0.002 | 0.012 | 0.002 | 0.003 | 0.004 | 0.027 | 0.002 |
| −5.0 | 0.019 | 0.002 | 0.021 | 0.000 | 0.008 | 0.005 | 0.021 | 0.000 |
| −5.4 | 0.019 | 0.005 | 0.019 | 0.002 | 0.019 | 0.002 | 0.018 | 0.004 |
| −5.9 | 0.021 | 0.004 | 0.025 | 0.004 | 0.018 | 0.000 | 0.018 | 0.000 |
| −6.4 | 0.023 | 0.002 | 0.021 | 0.000 | 0.021 | 0.000 | 0.016 | 0.002 |
| −6.9 | 0.016 | 0.002 | 0.019 | 0.002 | 0.021 | 0.000 | 0.016 | 0.002 |
| −7.3 | 0.019 | 0.002 | 0.021 | 0.004 | 0.021 | 0.004 | 0.018 | 0.000 |
| −7.8 | 0.023 | 0.002 | 0.019 | 0.005 | 0.021 | 0.004 | 0.014 | 0.000 |
| −8.3 | 0.021 | 0.000 | 0.019 | 0.002 | 0.023 | 0.002 | 0.016 | 0.002 |

Compound

| | | | |
|---|---|---|---|
| 5-16 | 5-17 | 5-18 | 5-20 |

| Conc. (Log[M]) | Average | SEM | Average | SEM | Average | SEM | Average | SEM |
|---|---|---|---|---|---|---|---|---|
| −4.0 | 0.030 | 0.000 | 0.016 | 0.002 | 0.018 | 0.002 | 0.021 | 0.000 |
| −4.5 | 0.020 | 0.002 | 0.018 | 0.000 | 0.008 | 0.002 | 0.021 | 0.000 |
| −5.0 | 0.010 | 0.002 | 0.019 | 0.002 | 0.021 | 0.000 | 0.011 | 0.000 |
| −5.4 | 0.020 | 0.002 | 0.016 | 0.005 | 0.021 | 0.000 | 0.018 | 0.007 |
| −5.9 | 0.020 | 0.002 | 0.018 | 0.002 | 0.021 | 0.004 | 0.016 | 0.000 |
| −6.4 | 0.013 | 0.000 | 0.018 | 0.002 | 0.021 | 0.004 | 0.011 | 0.000 |
| −6.9 | 0.013 | 0.005 | 0.011 | 0.000 | 0.011 | 0.000 | 0.018 | 0.002 |
| −7.3 | 0.013 | 0.000 | 0.018 | 0.002 | 0.018 | 0.002 | 0.018 | 0.002 |
| −7.8 | 0.013 | 0.000 | 0.020 | 0.000 | 0.020 | 0.000 | 0.018 | 0.002 |
| −8.3 | 0.025 | 0.002 | 0.013 | 0.002 | 0.013 | 0.002 | 0.016 | 0.000 |

| Compound | | |
|---|---|---|
| 5-21 | 5-22 | 5-24 |

| Conc. (Log[M]) | Average | SEM | Average | SEM | Average | SEM |
|---|---|---|---|---|---|---|
| −4.0 | 0.018 | 0.002 | 0.020 | 0.000 | 0.021 | 0.000 |
| −4.5 | 0.016 | 0.000 | 0.018 | 0.002 | 0.012 | 0.002 |
| −5.0 | 0.020 | 0.000 | 0.013 | 0.002 | 0.021 | 0.000 |
| −5.4 | 0.018 | 0.002 | 0.013 | 0.002 | 0.019 | 0.002 |
| −5.9 | 0.018 | 0.007 | 0.011 | 0.000 | 0.011 | 0.000 |
| −6.4 | 0.016 | 0.005 | 0.016 | 0.002 | 0.013 | 0.002 |
| −6.9 | 0.018 | 0.002 | 0.016 | 0.002 | 0.013 | 0.002 |
| −7.3 | 0.016 | 0.000 | 0.018 | 0.000 | 0.013 | 0.002 |
| −7.8 | 0.020 | 0.000 | 0.014 | 0.000 | 0.008 | 0.002 |
| −8.3 | 0.018 | 0.002 | 0.016 | 0.002 | 0.011 | 0.005 |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

Among other things, the present disclosure provides the following Embodiments:
1. A compound having the structure of formula I:

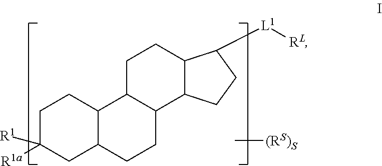

or a salt thereof, wherein:
   each of R¹ and R^{1a} is independently R^{s};
   each R is independently —H, -L″-R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, —N(R′)₂, a protected hydroxyl group, or R^{s} is

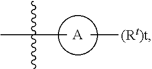

or two R^{s} attached to the same atom are taken together to form ═O or ═NR;
   t is 0-6;
   each R^{t} is independently R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, or —N(R′)₂;
   each Ring A is independently an optionally substituted 3-20 membered monocyclic, bicyclic or polycyclic ring having 0-10 heteroatoms;
   each L″ is independently a covalent bond, or an optionally substituted, bivalent C₁₋₆ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   R^{L} is R^{s}, —C(O)R^{s}, —C(O)OR^{s}, —C(O)N(R^{s})₂, —C(O)N(R′)S(O)₂R^{s}, —C(O)N(R′)C(R′)₂C(O)N(R′)₂, —C(O)N(R′)C(R′)₂S(O)₂R′, —C(O)N(R′)C(R′)₂S(O)₂N(R′)₂, —C(O)N(R′)C(R′)₂P(O)(R^{s})₂, —C(O)N(R′)C(R′)₂N(R′)C(O)N(R′)S(O)₂R^{s}, —C(O)N(R′)C(R^{s})₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂OR^{s}, —N(R^{s})₂, —N(R′)C(O)R^{s}, —N(R′)C(S)R^{s}, —N(R′)C(NR′)R′, —N(R′)C(O)OR′, —N(R′)C(O)N(R′)₂, —N(R′)C(NR′)N(R′)₂, —N(R′)C(O)N(R′)S(O)₂R^{s}, —N(R′)C(S)N(R′)S(O)₂R^{s}, —N(R′)C(NR′)N(R′)S(O)₂R^{s}, —N(R′)C(O)C(O)N(R′)S(O)₂R′, —N(R′)C(O)N(R′)S(O)₂N(R′)₂, —N(R′)S(O)₂R^{s}, —OC(O)N(R′)₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s};
   s is 0-25;
   R is -L-R′, —Si(R′)₃, or a hydroxyl protecting group;
   L¹ is L;
   each L is independently a covalent bond, or an optionally substituted, bivalent C₁₋₁₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   each -Cy- is independently an optionally substituted bivalent, 3-20 membered, monocyclic, bicyclic or polycyclic ring having 0-10 heteroatoms;
   R′ is R, —OR, —C(O)R, —C(O)OR, —C(O)N(R)₂, or —S(O)₂R; and each R is independently —H, or an optionally substituted group selected from C₁₋₁₅ aliphatic, C₁₋₁₅ heteroaliphatic having 1-5 heteroatoms, C₆₋₁₄ aryl, C₆₋₁₄ aryl-C₁₋₁₅ aliphatic, C₆₋₁₄ aryl-C₁₋₁₅ heteroaliphatic having 1-5 heteroatoms, C₁₋₁₅ aliphatic-C₆₋₁₄ aryl, C₁₋₁₅ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₅ aliphatic, 5-14 membered heteroaryl having 1-10 heteroatoms-C₁₋₁₅ heteroaliphatic having 1-5 heteroatoms, C₁₋₁₅ aliphatic-5-14 membered heteroaryl having 1-10 heteroatoms, C₁₋₁₅ heteroaliphatic having 1-5 heteroatoms-5-14 membered heteroaryl having 1-10 heteroatoms, C₂₋₂₀ biaryl having 0-10 heteroatoms, and 3-20 membered heterocyclyl having 1-5 heteroatoms, or
   two R groups are optionally and independently taken together to form a covalent bond or ═O, or:
   two or more R groups on the same atom are optionally and independently taken together with the atom to form an optionally substituted, 3-20 membered, monocyclic, bicyclic or polycyclic ring having, in addition to the atom, 0-5 heteroatoms; or
   two or more R groups on two or more atoms are optionally and independently taken together with their intervening atoms to form an optionally substituted, 3-20 membered, monocyclic, bicyclic or polycyclic ring having, in addition to the intervening atoms, 0-5 heteroatoms.
2. The compound of Embodiment 1, wherein s is 1-25 (e.g., 1, 2, 3, 4, 5, 6, 7, 8, 9, 10, 11, 12, 13, 14, 15, 16, 17, 18, 19, 20, 21, 22, 23, 24, or 25).
3. The compound of Embodiment 1, wherein s is 10-15.
4. The compound of Embodiment 1, wherein s is 5-10.
5. A compound having the structure of formula II:

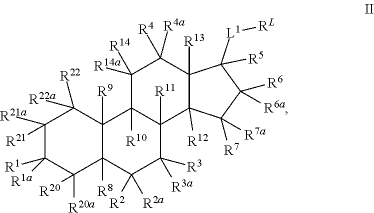

or a salt thereof, wherein:
   each of R′ R^{1a}, R², R^{2a}, R³, R^{3a}, R⁴, R⁴, R⁵, R⁶, R^{6a}, R⁷, R^{7a}, R⁸, R⁹, R¹⁰, R¹¹, R¹², R¹³, R¹⁴, R^{14a}, R²⁰, R^{20a}, R²¹, R^{21a}, R²² and R^{22a} is independently R^{s}; each R^{s} is independently —H, -L″-R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, —N(R′)₂, a protected hydroxyl group, or R^{s} is

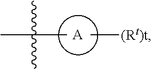

or two R^{s} attached to the same atom are taken together to form ═O or ═NR^{x};
   t is 0-6;
   each R^{t} is independently R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, or —N(R′)₂;
   each Ring A is independently an optionally substituted 3-20 membered monocyclic, bicyclic or polycyclic ring having 0-10 heteroatoms;
   each L″ is independently a covalent bond, or an optionally substituted, bivalent C₁₋₆ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   R^{L} is R^{s}, —C(O)R^{s}, —C(O)OR^{s}, —C(O)N(R^{s})₂, —C(O)N(R′)S(O)₂R^{s}, —C(O)N(R′)C(R′)₂C(O)N(R′)₂, —C(O)N(R′)C(R′)₂S(O)₂R′, —C(O)N(R′)C(R′)₂S(O)₂N(R′)₂, —C(O)N(R′)C(R′)₂P(O)(R^{s})₂, —C(O)N(R′)C(R′)₂N(R′)C(O)N(R′)S(O)₂R^{s}, —C(O)N(R′)C(R^{s})₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂OR^{s}, —N(R^{s})₂, —N(R′)C(O)R^{s}, —N(R′)C(S)R^{s}, —N(R′)C(NR′)R′, —N(R′)C(O)OR′, —N(R′)C(O)N(R′)₂, —N(R′)C(NR′)N(R′)₂, —N(R′)C(O)N(R′)S(O)₂R^{s}, —N(R′)C(S)N(R′)S(O)₂R^{s}, —N(R′)C(NR′)N(R′)S(O)₂R^{s}, —N(R′)C(O)C(O)N(R′)S(O)₂R′, —N(R′)C(O)N(R′)S(O)₂N(R′)₂, —N(R′)S(O)₂R^{s}, —OC(O)N(R′)₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s};
   R^{x} is -L-R′, —Si(R′)₃, or a hydroxyl protecting group;
   L¹ is L;
   each L is independently a covalent bond, or an optionally substituted, bivalent C₁₋₁₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   each -Cy- is independently an optionally substituted bivalent, 3-20 membered, monocyclic, bicyclic or polycyclic ring having 0-10 heteroatoms;
   R′ is R, —OR, —C(O)R, —C(O)OR, —C(O)N(R)₂, or —S(O)₂R; and each R is independently —H, or an optionally substituted group selected from C₁₋₁₅ aliphatic, C₁₋₁₅ heteroaliphatic having 1-5 heteroatoms, C₆₋₁₄ aryl, C₆₋₁₄ aryl-C₁₋₁₅ aliphatic, C₆₋₁₄ aryl-C₁₋₁₅ heteroaliphatic having 1-5 heteroatoms, C₁₋₁₅ aliphatic-C₆₋₁₄ aryl, C₁₋₁₅ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₅ aliphatic, 5-14 membered heteroaryl having 1-10 heteroatoms-C₁₋₁₅ heteroaliphatic having 1-5 heteroatoms, C₁₋₁₅ aliphatic-5-14 membered heteroaryl having 1-10 heteroatoms, C₁₋₁₅ heteroaliphatic having 1-5 heteroatoms-5-14 membered heteroaryl having 1-10 heteroatoms, C₂₋₂₀ biaryl having 0-10 heteroatoms, and 3-20 membered heterocyclyl having 1-5 heteroatoms, or
   two R groups are optionally and independently taken together to form a covalent bond or ═O, or:
   two or more R groups on the same atom are optionally and independently taken together with the atom to form an optionally substituted, 3-20 membered, monocyclic, bicyclic or polycyclic ring having, in addition to the atom, 0-5 heteroatoms; or
   two or more R groups on two or more atoms are optionally and independently taken together with their intervening atoms to form an optionally substituted, 3-20 membered, monocyclic, bicyclic or polycyclic ring having, in addition to the intervening atoms, 0-5 heteroatoms.
6. The compound of Embodiment 5, wherein the compound has the structure of formula II-a:

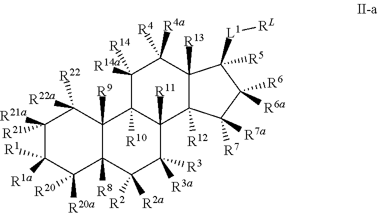

or salt thereof
7. The compound of Embodiment 5, wherein the compound has the structure of formula II-a′:

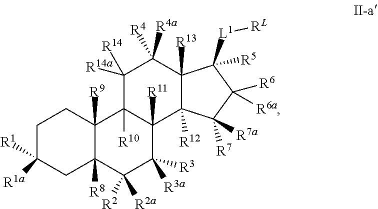

or salt thereof.
8. The compound of Embodiment 5, wherein the compound has the structure of formula II-b:

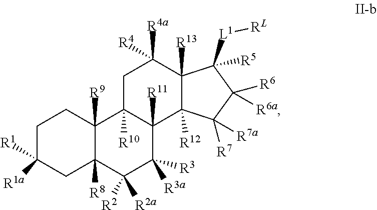

or salt thereof
9. The compound of Embodiment 5, wherein the compound has the structure of formula II-c:

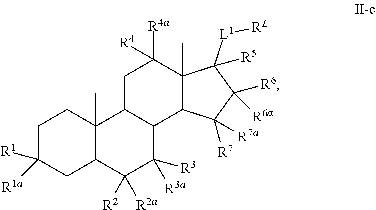

or salt thereof.
10. The compound of Embodiment 5, wherein the compound has the structure of formula II-d:

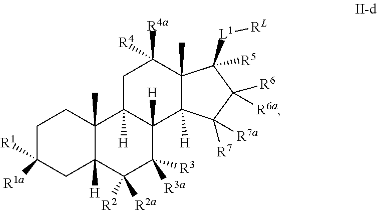

or salt thereof
11. The compound of Embodiment 5, wherein the compound has the structure of formula II-e:

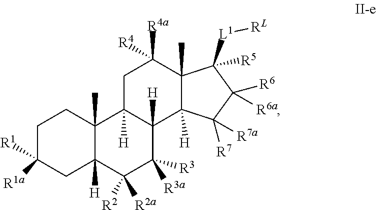

or salt thereof
12. The compound of Embodiment 5, wherein the compound has the structure of formula II-f:

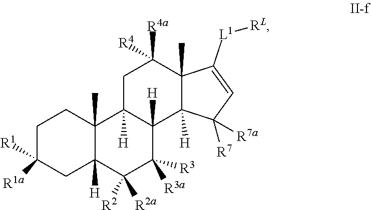

or salt thereof
13. The compound of Embodiment 5, wherein the compound has the structure of formula II-g:

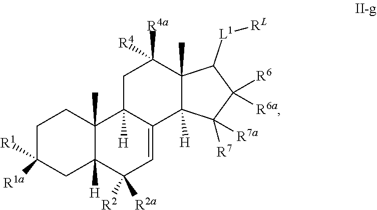

or salt thereof
14. The compound of Embodiment 5, wherein the compound has the structure of formula II-h:

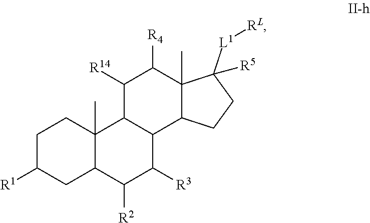

or salt thereof
15. The compound of Embodiment 5, wherein the compound has the structure of formula II-i:

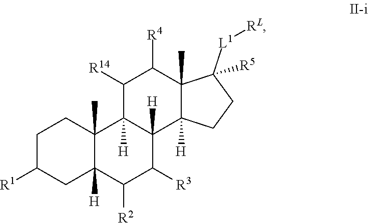

or salt thereof.
16. The compound of Embodiment 5, wherein the compound has the structure of formula II-j:

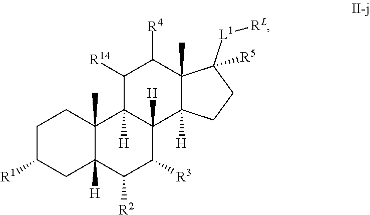

or salt thereof.
17. The compound of Embodiment 5, wherein the compound has the structure of formula II-k:

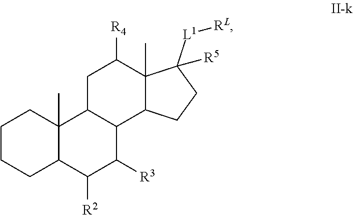

or salt thereof
18. The compound of Embodiment 5, wherein the compound has the structure of formula II-1:

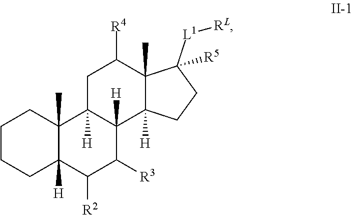

or salt thereof
19. The compound of Embodiment 5, wherein the compound has the structure of formula II-m:

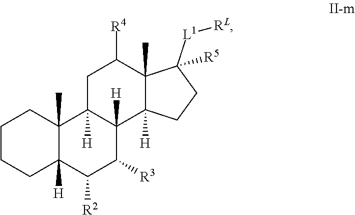

or salt thereof.
20. The compound of Embodiment 5, wherein the compound has the structure of formula II-n:

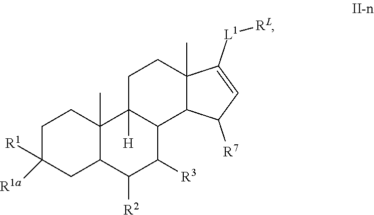

or salt thereof.
21. The compound of Embodiment 5, wherein the compound has the structure of formula II-o:

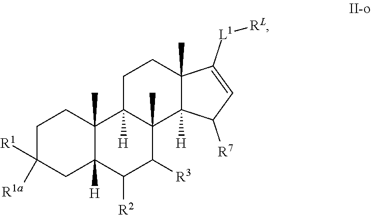

or salt thereof
22. The compound of Embodiment 5, wherein the compound has the structure of formula II-p:

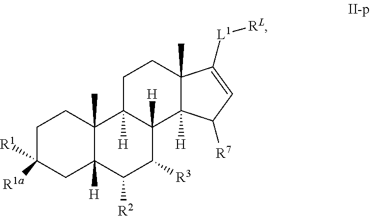

or salt thereof
23. The compound of Embodiment 5, wherein the compound has the structure of formula II—:

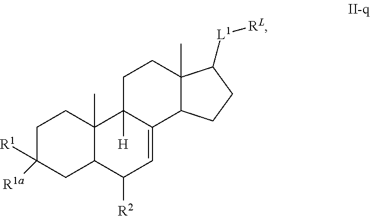

or salt thereof
24. The compound of Embodiment 5, wherein the compound has the structure of formula II-r:

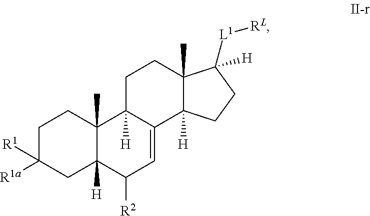

or salt thereof
25. The compound of Embodiment 5, wherein the compound has the structure of formula II-s:

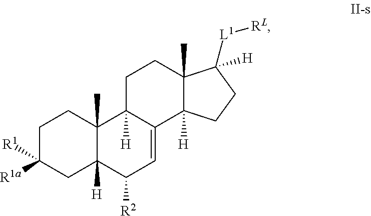

or salt thereof
26. The compound of any one of the preceding Embodiments, wherein R¹ is halogen.
27. The compound of any one of the preceding Embodiments, wherein R¹ is —F.
28. The compound of any one of Embodiments 1-25, wherein R¹ is —H.
29. The compound of Embodiment 28, wherein R¹ is -D.
30. The compound of any one of Embodiments 1-25, wherein R¹ is optionally substituted C₁₋₆ aliphatic.
31. The compound of Embodiment 28, wherein R¹ is optionally substituted C₁₋₆ alkyl.
32. The compound of Embodiment 31, wherein R¹ is methyl.
33. The compound of any one of Embodiments 1-25, wherein R¹ is —OR′.
34. The compound of Embodiment 33, wherein R¹ is —OH.
35. The compound of Embodiment 33, wherein R¹ is —OC(O)R.
36. The compound of Embodiment 35, wherein R¹ is —OC(O)R wherein R is —H or optionally substituted C₁₋₆ aliphatic.
37. The compound of Embodiment 36, wherein R¹ is —OC(O)H.
38. The compound of Embodiment 36, wherein R¹ is —OC(O)CH₃.
39. The compound of Embodiment 33, wherein R¹ is —OR′ wherein R′ is optionally substituted C₁₋₆ aliphatic.
40. The compound of Embodiment 39, wherein R¹ is —OR′ wherein R′ is optionally substituted C₁₋₆ alkyl.
41. The compound of Embodiment 40, wherein R¹ is —OMe.
42. The compound of Embodiment 33, wherein R¹ is —OR′ wherein R′ is —S(O)₂R.
43. The compound of Embodiment 33, wherein R¹ is —SO₃H.
44. The compound of any one of Embodiments 1-25, wherein R¹ is —N(R′)₂.
45. The compound of any one of Embodiments 1-25, wherein R¹ is —NH₂.
46. The compound of any one of the preceding Embodiments, wherein R^{1a} is —H.
47. The compound of Embodiment 46, wherein R^{1a} is -D.
48. The compound of any one of Embodiments 1-45, wherein R^{1a} is halogen.
49. The compound of Embodiment 49, wherein R^{1a} is —F.
50. The compound of any one of Embodiments 1-45, wherein R^{1a} is —OR′.
51. The compound of Embodiment 50, wherein R^{1a} is —OR′ wherein R′ is optionally substituted C₁₋₆ aliphatic.
52. The compound of Embodiment 51, wherein R^{1a} is —OR′ wherein R′ is optionally substituted C₁₋₆ alkyl.
53. The compound of Embodiment 52, wherein R^{1a} is —OMe.
54. The compound of any one of Embodiments 1-25, wherein R¹ and R^{1a} are taken together with their intervening atom to form an optionally substituted 3-20 membered monocyclic, bicyclic or polycyclic ring having 0-10 heteroatoms.
55. The compound of any one of Embodiments 1-25, wherein R¹ and R^{1a} are taken together to form ═O.
56. The compound of any one of the preceding Embodiments, wherein R² is —H.
57. The compound of any one Embodiments 1-55, wherein R² is halogen.
58. The compound of any one Embodiments 1-55, wherein R² is optionally substituted C₁₋₁₀ aliphatic.
59. The compound of Embodiment 58, wherein R² is optionally substituted C₁₋₈ alkyl.
60. The compound of Embodiment 58, wherein R² is C₁₋₈ alkyl.
61. The compound of Embodiment 58, wherein R² is optionally substituted C₁₋₄ alkyl.
62. The compound of Embodiment 58, wherein R² is C₁₋₄ alkyl.
63. The compound of Embodiment 62, wherein R² is ethyl.
64. The compound of Embodiment 58, wherein R² is optionally substituted C₂₋₈ alkenyl.
65. The compound of Embodiment 58, wherein R² is optionally substituted C₂₋₈ alkynyl.
66. The compound of Embodiment 58, wherein R² is optionally substituted C₃₋₈ cycloalkyl.
67. The compound of any one of the preceding Embodiments, wherein R^{2a} is —H.
68. The compound of any one Embodiments 1-66, wherein R^{2a} is halogen.
69. The compound of any one Embodiments 1-66, wherein R^{2a} is optionally substituted C₁₋₁₀ aliphatic.
70. The compound of Embodiment 69, wherein R^{2a} is optionally substituted C₁₋₈ alkyl.
71. The compound of Embodiment 69, wherein R^{2a} is C₁₋₈ alkyl.
72. The compound of Embodiment 69, wherein R^{2a} is optionally substituted C₁₋₄ alkyl.
73. The compound of Embodiment 69, wherein R^{2a} is C₁₋₄ alkyl.
74. The compound of Embodiment 69, wherein R^{2a} is ethyl.
75. The compound of Embodiment 69, wherein R^{2a} is optionally substituted C₂₋₈ alkenyl.
76. The compound of Embodiment 69, wherein R^{2a} is optionally substituted C₂₋₈ alkynyl.
77. The compound of Embodiment 69, wherein R^{2a} is optionally substituted C₃₋₈ cycloalkyl.
78. The compound of any one Embodiments 1-55, wherein R² and R^{2a} are taken together to form ═O.
79. The compound of any one Embodiments 1-55, wherein R² and R^{2a} are taken together with their intervening atom to form an optionally substituted 3-20 membered monocyclic, bicyclic or polycyclic ring having 0-10 heteroatoms.
80. The compound of any one of the preceding Embodiments, wherein R³ is —H.
81. The compound of any one of Embodiments 1-79, wherein R³ is R′.
82. The compound of Embodiment 81, wherein R³ is —OR.
83. The compound of Embodiment 82, wherein R³ is —OH.
84. The compound of Embodiment 81, wherein R³ is —OC(O)R.
85. The compound of Embodiment 84, wherein R³ is —OC(O)R wherein R is optionally substituted phenyl.
86. The compound of Embodiment 85, wherein R³ is —OC(O)R wherein R is 3-iodophenyl.
87. The compound of any one of the preceding Embodiments, wherein R^{3a} is —H.
88. The compound of any one of Embodiments 1-86, wherein R^{3a} is R′.
89. The compound of Embodiment 88, wherein R^{3a} is —OR.
90. The compound of Embodiment 88, wherein R^{3a} is —OH.
91. The compound of Embodiment 88, wherein R^{3a} is —OC(O)R.
92. The compound of Embodiment 88, wherein R^{3a} is —OC(O)R wherein R is optionally substituted phenyl.
93. The compound of Embodiment 88, wherein R^{3a} is —OC(O)R wherein R is 3-iodophenyl.
94. The compound of any one of Embodiments 1-79, wherein R³ and R^{3a} are taken together to form ═NR^{x}.
95. The compound of Embodiment 94, wherein R^{x} is —R′.
96. The compound of Embodiment 95, wherein R^{x} is —OR wherein R is optionally substituted C₁₋₆ aliphatic.
97. The compound of Embodiment 95, wherein R^{x} is —OR wherein R is optionally substituted C₁₋₆ alkyl.
98. The compound of Embodiment 95, wherein R^{x} is —OR wherein R is methyl.
99. The compound of Embodiment 94, wherein R^{x} is -L-R′.
100. The compound of Embodiment 99, wherein R^{x} is -L-R′ wherein L is an optionally substituted, bivalent C₁-C₁₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
101. The compound of Embodiment 100, wherein R^{x} is —O—C(R′)₂—C(O)O—R′.
102. The compound of Embodiment 101, wherein R^{x} is —O—C(CH₃)₂—C(O)OH.
103. The compound of any one Embodiments 1-79, wherein R³ and R^{3a} are taken together with their intervening atom to form an optionally substituted 3-20 membered monocyclic, bicyclic or polycyclic ring having 0-10 heteroatoms.
104. The compound of any one of Embodiments 1-79, wherein R³ and R^{3a} are taken together to form ═O.
105. The compound of any one of the preceding Embodiments, wherein R⁴ is —H.
106. The compound of any one of Embodiments 1-104, wherein R⁴ is —OH.
107. The compound of any one of Embodiments 1-104, wherein R⁴ is optionally substituted C₁₋₆ aliphatic.
108. The compound of Embodiment 107, wherein R⁴ is optionally substituted C₁₋₆ alkyl.
109. The compound of Embodiment 108, wherein R⁴ is methyl.
110. The compound of any one of the preceding Embodiments, wherein R^{4a} is —H.
111. The compound of any one of Embodiments 1-109, wherein R⁴ is —OH.
112. The compound of any one of Embodiments 1-109, wherein R⁴ is optionally substituted C₁₋₆ aliphatic.
113. The compound of Embodiment 112, wherein R⁴ is optionally substituted C₁₋₆ alkyl.
114. The compound of Embodiment 113, wherein R⁴ is methyl.
115. The compound of any one of Embodiments 1-104, wherein R⁴ and R^{4a} are taken together to form ═O.
116. The compound of any one of the preceding Embodiments, wherein R⁵ is R′.
117. The compound of Embodiment 116, wherein R⁵ is —H.
118. The compound of any one of Embodiments 1-115, wherein R⁵ is —OR′.
119. The compound of Embodiment 118, wherein R⁵ is —OH.
120. The compound of any one of Embodiments 1-115, wherein R⁵ is halogen.
121. The compound of any one of the preceding Embodiments, wherein R⁶ is —H.
122. The compound of any one of Embodiments 1-120, wherein R⁶ is halogen.
123. The compound of any one of Embodiments 1-120, wherein R⁶ is R′.
124. The compound of any one of Embodiments 1-120, wherein R⁵ and R⁶ are taken together to form a covalent bond.
125. The compound of any one of the preceding Embodiments, wherein R^{6a} is —H.
126. The compound of any one of Embodiments 1-124, wherein R^{6a} is halogen.
127. The compound of any one of Embodiments 1-124, wherein R^{6a} is R′.
128. The compound of any one of the preceding Embodiments, wherein R⁷ is —R′.
129. The compound of any one of the preceding Embodiments, wherein R⁷ is —H.
130. The compound of any one of Embodiments 1-125, wherein R⁷ is halogen.
131. The compound of any one of Embodiments 1-125, wherein R⁷ is —OR.
132. The compound of Embodiment 131, wherein R⁷ is —OH.
133. The compound of any one of the preceding Embodiments, wherein R^{7a} is —R′.
134. The compound of any one of Embodiments 1-132, wherein R^{7a} is —H.
135. The compound of any one of Embodiments 1-132, wherein R^{7a} is halogen.
136. The compound of any one of Embodiments 1-132, wherein R^{7a} is —OR.
137. The compound of Embodiment 136, wherein R^{7a} is —OH.
138. The compound of any one of Embodiments 1-125, wherein R⁷ and R^{7a} are taken together to form ═O.
139. The compound of any one of the preceding Embodiments, wherein R⁸ is —H or optionally substituted C₁₋₆ aliphatic.
140. The compound of any one of the preceding Embodiments, wherein R⁸ is —H.
141. The compound of any one of the preceding Embodiments, wherein R⁹ is —H or optionally substituted C₁₋₆ aliphatic.
142. The compound of any one of the preceding Embodiments, wherein R⁹ is optionally substituted C₁₋₆ aliphatic.
143. The compound of any one of the preceding Embodiments, wherein R⁹ is methyl.
144. The compound of any one of the preceding Embodiments, wherein R¹⁰ is —H or optionally substituted C₁₋₆ aliphatic.
145. The compound of any one of the preceding Embodiments, wherein R¹⁰ is —H.
146. The compound of any one of the preceding Embodiments, wherein R¹¹ is —H or optionally substituted C₁₋₆ aliphatic.
147. The compound of any one of the preceding Embodiments, wherein R¹¹ is —H.
148. The compound of any one of Embodiments 1-145, wherein R¹¹ and R³ are taken together to form a covalent bond.
149. The compound of any one of the preceding Embodiments, wherein R¹² is —H or optionally substituted C₁₋₆ aliphatic.
150. The compound of any one of the preceding Embodiments, wherein R¹² is —H.
151. The compound of any one of the preceding Embodiments, wherein R¹³ is —H or optionally substituted C₁₋₆ aliphatic.
152. The compound of any one of the preceding Embodiments, wherein R¹³ is optionally substituted C₁₋₆ aliphatic.
153. The compound of any one of the preceding Embodiments, wherein R¹³ is methyl.
154. The compound of any one of the preceding Embodiments, wherein R¹⁴ is —H.
155. The compound of any one of Embodiments 1-153, wherein R¹⁴ is halogen.
156. The compound of any one of Embodiments 1-153, wherein R¹⁴ is R′.
157. The compound of any one of Embodiments 1-153, wherein R¹⁴ is optionally substituted C₁₋₆ aliphatic.
158. The compound of any one of the preceding Embodiments, wherein R^{14a} is —H.
159. The compound of any one of Embodiments 1-157, wherein R^{14a} is halogen.
160. The compound of any one of Embodiments 1-157, wherein R^{14a} is R′.
161. The compound of any one of Embodiments 1-157, wherein R^{14a} is optionally substituted C₁₋₆ aliphatic.
162. The compound of any one of Embodiments 1-157, wherein R^{14a} and R^{4a} are taken together to form a covalent bond.
163. The compound of any one of the preceding Embodiments, wherein R²⁰ is —H.
164. The compound of any one of Embodiments 1-162, wherein R²⁰ is halogen.
165. The compound of any one of Embodiments 1-162, wherein R²⁰ is R′.
166. The compound of any one of Embodiments 1-162, wherein R²⁰ is optionally substituted C₁₋₆ aliphatic.
167. The compound of any one of the preceding Embodiments, wherein R^{20a} is —H.
168. The compound of any one of Embodiments 1-166, wherein R^{20a} is halogen.
169. The compound of any one of Embodiments 1-166, wherein R^{20a} is R′.
170. The compound of any one of Embodiments 1-166, wherein R^{20a} is optionally substituted C₁₋₆ aliphatic.
171. The compound of any one of the preceding Embodiments, wherein R²¹ is —H.
172. The compound of any one of Embodiments 1-170, wherein R²¹ is halogen.
173. The compound of any one of Embodiments 1-170, wherein R²¹ is R′.
174. The compound of any one of Embodiments 1-170, wherein R²¹ is optionally substituted C₁₋₆ aliphatic.
175. The compound of any one of the preceding Embodiments, wherein R^{21a} is —H.
176. The compound of any one of Embodiments 1-174, wherein R^{21a} is halogen.
177. The compound of any one of Embodiments 1-174, wherein R^{21a} is R′.
178. The compound of any one of Embodiments 1-174, wherein R^{21a} is optionally substituted C₁₋₆ aliphatic.
179. The compound of any one of Embodiments 1-174, wherein R^{21a} and R^{1a} are taken together to form a covalent bond.
180. The compound of any one of Embodiments 1-174, wherein R^{21a} and R^{1a} are taken together to form a ring.
181. The compound of any one of Embodiments 1-174, wherein R^{21a} and R^{1a} are taken together with their intervening atoms to form an optionally substituted 5-6 membered aromatic ring having 0-4 heteroatoms.
182. The compound of any one of Embodiments 1-174, wherein R^{21a} and R^{1a} are taken together with their intervening atoms to form an optionally substituted 5-6 membered heteroaryl ring having 1-4 heteroatoms.
183. The compound of any one of the preceding Embodiments, wherein R²² is —H.
184. The compound of any one of Embodiments 1-182, wherein R²² is halogen.
185. The compound of any one of Embodiments 1-182, wherein R²² is R′.
186. The compound of any one of Embodiments 1-182, wherein R²² is optionally substituted C₁₋₆ aliphatic.
187. The compound of any one of the preceding Embodiments, wherein R^{22a} is —H.
188. The compound of any one of Embodiments 1-186, wherein R^{22a} is halogen.
189. The compound of any one of Embodiments 1-186, wherein R^{22a} is R′.
190. The compound of any one of Embodiments 1-186, wherein R^{22a} is optionally substituted C₁₋₆ aliphatic.
191. The compound of any one of Embodiments 1-186, wherein R^{22a} and R^{4a} are taken together to form a double bond.
192. The compound of any one of the preceding Embodiments, wherein each L¹ is an optionally substituted, bivalent C₁-C₁₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
193. The compound of any one of the preceding Embodiments, wherein each L¹ is an optionally substituted, bivalent C₁-C₁₂ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
194. The compound of any one of the preceding Embodiments, wherein each L¹ is an optionally substituted, bivalent C₁-C₁₂ alkylene group wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
195. The compound of any one of the preceding Embodiments, wherein each L¹ is —CH₂-L¹-, wherein —CH₂— is optionally substituted, L¹ is bonded to R^{L}, and L¹ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
196. The compound of any one of the preceding Embodiments, wherein each L¹ is —CHR′-L′-, wherein L¹ is bonded to R^{L}, and L¹ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
197. The compound of Embodiment 196, wherein R′ of —CHR′— is optionally substituted C₁₋₆ aliphatic.
198. The compound of Embodiment 197, wherein R′ of —CHR′— is optionally substituted C₁₋₆ alkyl.
199. The compound of any one of the preceding Embodiments, wherein each L¹ is —CH(CH₃)-L¹-, wherein —CH(CH₃)— is optionally substituted, L¹ is bonded to R^{L}, and L¹ is a covalent bond or an optionally substituted, bivalent C₁-C₁₁ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
200. The compound of any one of Embodiments 195-199, wherein L¹ is -L^{s1}-L^{s2}-, wherein L^{s2} is bonded to R^{L}, L^{s1} is a covalent bond or an optionally substituted, bivalent C₁-C₆ aliphatic or heteroaliphatic group having 1-5 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, and L^{s2} is a covalent bond or an optionally substituted, bivalent C₁-C₅ aliphatic or heteroaliphatic group having 1-5 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
201. The compound of Embodiment 200, wherein L^{s1} is a covalent bond or an optionally substituted, bivalent C₁-C₆ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, and L^{s2} is a covalent bond or an optionally substituted, bivalent C₁-C₅ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
202. The compound of Embodiment 201, wherein L^{s1} is optionally substituted —(CH₂)n- wherein n is 1, 2, 3, 4, 5, or 6.
203. The compound of Embodiment 202, wherein L^{s1} is —(CH₂)n- wherein n is 1, 2, 3, 4, 5, or 6.
204. The compound of any one of Embodiments 202-203, wherein n is 1.
205. The compound of any one of Embodiments 202-203, wherein n is 2.
206. The compound of any one of Embodiments 202-203, wherein n is 3.
207. The compound of any one of Embodiments 202-203, wherein n is 4.
208. The compound of Embodiment 201, wherein L^{s1} is optionally substituted —(CH₂)n-C(R′)₂— wherein n is 1, 2, 3, 4, or 5.
209. The compound of Embodiment 201, wherein L^{s1} is —(CH₂)n-C(R′)₂— wherein n is 1, 2, 3, 4, or 5.
210. The compound of Embodiment 201, wherein L^{s1} is optionally substituted —(CH₂)n-CHR′— wherein n is 1, 2, 3, 4, or 5.
211. The compound of Embodiment 201, wherein L^{s1} is optionally substituted —(CH₂)n-CHR′— wherein n is 1, 2, 3, 4, or 5.
212. The compound of any one of Embodiments 208-211, wherein n is 1.
213. The compound of any one of Embodiments 208-211, wherein n is 2.
214. The compound of any one of Embodiments 208-211, wherein n is 3.
215. The compound of any one of Embodiments 208-211, wherein n is 4.
216. The compound of any one of Embodiments 208-215, wherein each R′ is independently —H or optionally substituted C₁₋₆ aliphatic.
217. The compound of Embodiment 216, wherein each R′ is independently optionally substituted C₁₋₆ aliphatic.
218. The compound of Embodiment 216, wherein each R′ is independently optionally substituted C₁₋₆ alkyl.
219. The compound of Embodiment 216, wherein each R′ is independently methyl.
220. The compound of Embodiment 216, wherein L^{s1} is —(CH₂)n-CH(CH₃)—.
221. The compound of Embodiment 216, wherein L^{s1} is —(CH₂)n-C(CH₃)₂—.
222. The compound of Embodiment 201, wherein L^{s1} is a covalent bond.
223. The compound of any one of the preceding Embodiments, wherein L^{s2} comprises —O—.
224. The compound of any one of the preceding Embodiments, wherein L^{s2} comprises —S(O)₂—.
225. The compound of any one of the preceding Embodiments, wherein L^{s2} comprises —C(O)—.
226. The compound of any one of Embodiments 223-225, wherein —(O)—, —S(O)₂—, or —C(O)— is bonded to L^{s1}.
227. The compound of any one of Embodiments 223-225, wherein —(O)—, —S(O)₂—, or —C(O)— is bonded to R^{L}.
228. The compound of any one of the preceding Embodiments, wherein L^{s2} comprises —C(O)O—.
229. The compound of any one of the preceding Embodiments, wherein L^{s2} comprises —C(O)N(R′)—.
230. The compound of any one of the preceding Embodiments, wherein L^{s2} comprises —C(O)N(R′)S(O)₂—.
231. The compound of any one of the preceding Embodiments, wherein L^{s2} comprises —N(R′)—.
232. The compound of any one of the preceding Embodiments, wherein L^{s2} comprises —N(R′)S(O)₂—.
233. The compound of any one of the preceding Embodiments, wherein L^{s2} comprises —N(R′)C(O)N(R′)S(O)₂—.
234. The compound of any one of the preceding Embodiments, wherein L^{s2} comprises —N(R′)C(O)N(R′)—.
235. The compound of any one of Embodiments 229-234, wherein each R′ is independently —H or optionally substituted C₁₋₆ aliphatic.
236. The compound of any one of Embodiments 229-234, wherein each R′ is independently —H.
237. The compound of any one of Embodiments 228-230, wherein —C(O)— is bonded to L^{s1}.
238. The compound of any one of Embodiments 228-230, wherein —C(O)— is bonded to R^{L}.
239. The compound of any one of Embodiments 231-236, wherein —N(R′)— is bonded to L^{s1}.
240. The compound of any one of Embodiments 231-236, wherein —N(R′)— is bonded to R^{L}.
241. The compound of any one of the preceding Embodiments, wherein L^{s2} is —C(O)O—.
242. The compound of Embodiment 241, wherein —O— is bonded to R^{L}.
243. The compound of any one of Embodiments 1-222, wherein L^{s2} is —C(O)N(R′)—.
244. The compound of Embodiment 243, wherein L^{s2} is —C(O)N(R′)— wherein R′ is —H or optionally substituted C₁₋₆ aliphatic.
245. The compound of Embodiment 243, wherein L^{s2} is —C(O)NH—.
246. The compound of any one of Embodiments 243-245, wherein —C(O)— is bonded to L^{s1}.
247. The compound of any one of Embodiments 1-222, wherein L^{s2} is —C(O)N(R′)S(O)₂—.
248. The compound of Embodiment 247, wherein L^{s2} is —C(O)N(R′)S(O)₂— wherein R′ is —H or optionally substituted C₁₋₆ aliphatic.
249. The compound of Embodiment 248, wherein L^{s2} is —C(O)NHS(O)₂—.
250. The compound of any one of Embodiments 248-249, wherein —S(O)₂— is bonded to R^{L}.
251. The compound of any one of Embodiments 1-222, wherein L^{s2} is —N(R′)C(O)N(R′)S(O)₂—.
252. The compound of Embodiment 251, wherein L^{s2} is —N(R′)C(O)N(R′)S(O)₂— wherein each R′ is independently —H or optionally substituted C₁₋₆ aliphatic.
253. The compound of Embodiment 252, wherein L^{s2} is —NHC(O)NHS(O)₂—.
254. The compound of any one of Embodiments 251-253, wherein —S(O)₂— is bonded to R^{L}.
255. The compound of any one of Embodiments 1-240, wherein L^{s2} comprises —N(R′)C(O)N(R′)S(O)₂-Cy-L″-, wherein L″ is a covalent bond, or an optionally substituted, bivalent C₁-C₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
256. The compound of Embodiment 255, wherein L^{s2} comprises —N(R′)C(O)N(R′)S(O)₂-Cy-L″— wherein each R′ is independently —H or optionally substituted C₁₋₆ aliphatic.
257. The compound of Embodiment 255, wherein L^{s2} comprises —NHC(O)NHS(O)₂-Cy-L″-.
258. The compound of any one of Embodiments 1-222, wherein L^{s2} is —N(R′)C(O)N(R′)S(O)₂-Cy-L″-, wherein L″ is a covalent bond, or an optionally substituted, bivalent C₁-C₂ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.
259. The compound of Embodiment 255, wherein L^{s2} is —N(R′)C(O)N(R′)S(O)₂-Cy-L″— wherein each R′ is independently —H or optionally substituted C₁₋₆ aliphatic.
260. The compound of Embodiment 259, wherein L^{s2} is —NHC(O)NHS(O)₂-Cy-L″-.
261. The compound of any one of Embodiments 255-260, wherein L″ is bonded to R^{L}.
262. The compound of any one of Embodiments 255-261, wherein L″ is a covalent bond.
263. The compound of any one of Embodiments 255-261, wherein L″ is optionally substituted —(CH₂)n- wherein n is 1 or 2.
264. The compound of Embodiment 263, wherein n is 1.
265. The compound of Embodiment 263, wherein n is 2.
266. The compound of any one of Embodiments 263-264, wherein L″ is —C(CH₃)₂—.
267. The compound of any one of Embodiments 263-264, wherein L″ is —C(CH₃)(CH₂OH)—.
268. The compound of any one of Embodiments 263-264, wherein L″ is —CH₂—.
269. The compound of any one of Embodiments 255-261, wherein L″ is —C(R′)₂—(CH₂)n-, wherein each —CH₂— is optionally substituted and n is 0 or 1.
270. The compound of any one of Embodiments 255-261, wherein L″ is —C(R′)₂—(CH₂)n-.
271. The compound of any one of Embodiments 269-270, wherein n is 0.
272. The compound of any one of Embodiments 269-270, wherein n is 1.
273. The compound of Embodiment 272, wherein the two R′ of —C(R′)₂— are taken together with the carbon atom to which they are attached to form an optionally substituted 3-10 membered cycloaliphatic ring.
274. The compound of Embodiment 273, wherein a formed ring is 3-membered.
275. The compound of Embodiment 273, wherein a formed ring is 4-membered.
276. The compound of Embodiment 273, wherein a formed ring is 5-membered.
277. The compound of Embodiment 273, wherein a formed ring is 6-membered.
278. The compound of any one of Embodiments 273-277, wherein a formed ring is saturated.
279. The compound of any one of Embodiments 1-240, wherein L^{s2} comprises -Cy-.
280. The compound of Embodiment 279, wherein -Cy- is bonded to L^{s1}.
281. The compound of Embodiment 279, wherein -Cy- is bonded to R^{L}.
282. The compound of any one of Embodiments 255-281, wherein -Cy- is optionally substituted phenylene.
283. The compound of Embodiment 282, wherein -Cy- is optionally substituted 1,4-phenylene.
284. The compound of any one of Embodiments 255-281, wherein -Cy- an optionally substituted bivalent C₃₋₁₀ cycloaliphatic ring.
285. The compound of Embodiment 284, wherein -Cy- an optionally substituted bivalent C₃₋₁₀ cycloalkyl ring.
286. The compound of Embodiment 285, wherein -Cy- an optionally substituted bivalent cyclohexyl ring.
287. The compound of Embodiment 285, wherein -Cy- optionally substituted bivalent cyclohexyl ring.
288. The compound of any one of Embodiments 255-281, wherein -Cy- is an optionally substituted bicyclic 5-10 membered ring.
289. The compound of Embodiment 288, wherein a monocyclic unit is aromatic.
290. The compound of Embodiment 289, wherein a monocyclic unit is an optionally substituted phenyl ring.
291. The compound of any one of Embodiments 255-281, wherein -Cy- is an optionally substituted monocyclic or bicyclic 3-10 membered heterocyclyl ring having 1-5 heteroatoms.
292. The compound of Embodiment 291, wherein the ring is 5-membered.
293. The compound of Embodiment 291, wherein the ring is 6-membered.
294. The compound of any one of Embodiments 291-293, wherein the ring is saturated.
295. The compound of any one of Embodiments 291-294, wherein the ring contains one and no more than one heteroatom.
296. The compound of any one of Embodiments 291-295, wherein the ring contains a nitrogen atom.
297. The compound of Embodiment 296, wherein the nitrogen atom is boned to —S(O)₂—.
298. The compound of any one of Embodiments 291-297, wherein the ring is monocyclic.
299. The compound of any one of Embodiments 291-297, wherein the ring is bicyclic.
300. The compound of any one of Embodiments 1-240, wherein L^{s2} comprises optionally substituted —CH═CH—.
301. The compound of any one of Embodiments 1-240, wherein L^{s2} comprises —CH═CH—.
302. The compound of any one of Embodiments 1-240, wherein L^{s2} comprises optionally substituted —CH═CH—C(O)O—.
303. The compound of Embodiment 300, wherein L^{s2} comprises —C(CH₃)=CH—C(O)O—.
304. The compound of Embodiment 300, wherein L^{s2} comprises —CH═CH—C(O)O—.
305. The compound of any one of Embodiments 1-222, wherein L^{s2} is optionally substituted —CH═CH—C(O)O—.
306. The compound of Embodiment 300, wherein L^{s2} is —C(CH₃)=CH—C(O)O—
307. The compound of Embodiment 300, wherein L^{s2} is —CH═CH—C(O)O—.
308. The compound of any one of Embodiments 300-307, wherein —O— is bonded to R^{L}.
309. The compound of any one of Embodiments 300-308, wherein —CH═CH— is E.
310. The compound of any one of Embodiments 300-308, wherein —CH═CH— is Z.
311. The compound of any one of Embodiments 1-222, wherein L^{S2} is —O—.
312. The compound of any one of Embodiments 1-222, wherein L^{s2} is —C(O)—.
313. The compound of any one of Embodiments 1-222, wherein L^{S2} is —O—S(O)₂—O—.
314. The compound of any one of Embodiments 1-222, wherein L^{s2} is —N(R′)—.
315. The compound of Embodiment 314, wherein L^{s2} is —NH—.
316. The compound of any one of Embodiments 1-222, wherein L^{s2} is a covalent bond.
317. The compound of any one of Embodiments 1-191, wherein L¹ is -L^{a}-L^{b}-L^{c}-, wherein:
   L^{a} is a covalent bond, or an optionally substituted, bivalent C₁₋₂ aliphatic or heteroaliphatic group having 1-2 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   L^{b} is a covalent bond, or an optionally substituted, bivalent C₁₋₁₀ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   L^{c} is a covalent bond, or an optionally substituted, bivalent C₁₋₃ aliphatic or heteroaliphatic group having 1-3 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—; and
   L^{c} is bonded to R^{L}.
318. The compound of Embodiment 317, wherein L^{a} is a covalent bond.
319. The compound of Embodiment 317, wherein L^{a} is optionally substituted —CH₂—.
320. The compound of Embodiment 317, wherein L^{a} is —C(R)₂—.
321. The compound of Embodiment 317, wherein L^{a} is —CHR—.
322. The compound of Embodiment 317, wherein L^{a} is —CH(CH₃)—.
323. The compound of Embodiment 317, wherein L^{a} is —(S)—CH(CH₃)—.
324. The compound of Embodiment 317, wherein L^{a} is —(R)—CH(CH₃)—.
325. The compound of any one of Embodiments 317-324, wherein L^{b} is a covalent bond.
326. The compound of any one of Embodiments 317-324, wherein L^{b} is optionally substituted bivalent C₁₋₁₀ aliphatic.
327. The compound of any one of Embodiments 317-324, wherein L^{b} is optionally substituted bivalent C₁₋₁₀ alkylene.
328. The compound of any one of Embodiments 317-324, wherein L^{b} is optionally substituted —(CH₂)₁₋₁₀—.
329. The compound of any one of Embodiments 317-324, wherein L^{b} is optionally substituted —CH₂—.
330. The compound of any one of Embodiments 317-324, wherein L^{b} is —CH₂—.
331. The compound of any one of Embodiments 317-324, wherein L^{b} is optionally substituted —(CH₂)₂—.
332. The compound of any one of Embodiments 317-324, wherein L^{b} is —(CH₂)₂—.
333. The compound of any one of Embodiments 317-324, wherein L^{b} is optionally substituted —(CH₂)₃—.
334. The compound of any one of Embodiments 317-324, wherein L^{b} is —(CH₂)₃—.
335. The compound of any one of Embodiments 317-324, wherein L^{b} is optionally substituted —(CH₂)₄—.
336. The compound of any one of Embodiments 317-324, wherein L is —(CH₂)₄.
337. The compound of any one of Embodiments 317-324, wherein L is —(CH₂)ₘ—CHR′—, wherein each —CH₂— is optionally substituted and m is 0-3.
338. The compound of any one of Embodiments 317-324, wherein L is —(CH₂)ₘ—CHR′— wherein m is 0-3.
339. The compound of any one of Embodiments 337-338, wherein R′ is 0.
340. The compound of any one of Embodiments 337-338, wherein R′ is 1.
341. The compound of any one of Embodiments 337-338, wherein in is 2.
342. The compound of any one of Embodiments 337-338, wherein is 3.
343. The compound of any one of Embodiments 337-342, wherein R′ in —CHR′— is —H.
344. The compound of any one of Embodiments 337-342, wherein R′ in —CHR′— is R.
345. The compound of any one of Embodiments 337-342, wherein R′ in —CHR′— is optionally substituted C₁₋₆ aliphatic.
346. The compound of any one of Embodiments 317-324, wherein L^{b} is —CH₂—O—.
347. The compound of any one of Embodiments 317-324, wherein L^{b} is —CH₂—OC(O)—.
348. The compound of any one of Embodiments 317-324, wherein L^{b} is —CH₂—OC(O)N(R′)—.
349. The compound of any one of Embodiments 317-324, wherein L^{b} is —CH₂—OC(O)NH—.
350. The compound of any one of Embodiments 317-324, wherein L^{b} is —CH₂—OC(O)N(R′)S(O)₂—.
351. The compound of any one of Embodiments 317-324, wherein L^{b} is —CH₂—OC(O)NHS(O)₂—.
352. The compound of any one of Embodiments 317-351, wherein L^{c} is a covalent bond.
353. The compound of any one of Embodiments 317-351, wherein L^{c} is optionally substituted bivalent C₁₋₁₀ aliphatic.
354. The compound of any one of Embodiments 317-351, wherein L^{c} is C₁₋₁₀ is optionally substituted alkylene.
355. The compound of any one of Embodiments 317-351, wherein L^{c} is optionally substituted —(CH₂)₁₋₁₀—.
356. The compound of any one of Embodiments 317-351, wherein L^{c} is optionally substituted —CH₂—.
357. The compound of any one of Embodiments 317-351, wherein L^{c} is optionally substituted —(CH₂)₂—.
358. The compound of any one of Embodiments 317-351, wherein L^{c} is optionally substituted —(CH₂)₃—. 359. The compound of any one of Embodiments 317-351, wherein L^{c} is —(CH₂)₁₋₁₀—.
360. The compound of any one of Embodiments 317-351, wherein L^{c} is —CH₂—.
361. The compound of any one of Embodiments 317-351, wherein L^{c} is —(CH₂)₂—.
362. The compound of any one of Embodiments 317-351, wherein L^{c} is —(CH₂)₃—.
363. The compound of any one of Embodiments 317-351, wherein L^{c} is —CH(COOH)—.
364. The compound of any one of Embodiments 317-351, wherein L^{c} is —CH(CN)—.
365. The compound of any one of Embodiments 317-351, wherein L^{c} is —C(R′)₂—.
366. The compound of any one of Embodiments 317-351, wherein L^{c} is —CHR′—.
367. The compound of any one of Embodiments 317-351, wherein L^{c} is —CH(CH₃)—.
368. The compound of any one of Embodiments 317-351, wherein L^{c} is —(R)—CH(CH₃)—.
369. The compound of any one of Embodiments 317-351, wherein L^{c} is —(S)—CH(CH₃)—.
370. The compound of any one of Embodiments 317-351, wherein L^{c} is -L″-.
371. The compound of any one of Embodiments 317-351, wherein L^{c} is —O—.
372. The compound of any one of Embodiments 317-351, wherein L^{c} is —C(O)—.
373. The compound of any one of Embodiments 317-351, wherein L^{c} is —S(O)₂—.
374. The compound of any one of Embodiments 317-351, wherein L^{c} is —N(R′)—.
375. The compound of any one of Embodiments 317-351, wherein L^{c} is —NH—.
376. The compound of any one of Embodiments 317-351, wherein L^{c} is —OC(O)—.
377. The compound of any one of Embodiments 317-351, wherein L^{c} is —OC(O)N(R′)—.
378. The compound of any one of Embodiments 317-351, wherein L^{c} is —OC(O)NH—.
379. The compound of any one of Embodiments 317-351, wherein L^{c} is —OC(O)N(R′)S(O)—.
380. The compound of any one of Embodiments 317-351, wherein L^{c} is —OC(O)NHS(O)₂—.
381. The compound of any one of Embodiments 317-351, wherein L^{c} is —OS(O)₂—.
382. The compound of any one of Embodiments 317-351, wherein L^{c} is —N(R′)S(O)₂—.
383. The compound of any one of Embodiments 317-351, wherein L^{c} is —NHS(O)₂—.
384. The compound of any one of Embodiments 317-351, wherein L^{c} is —C(O)N(R′)S(O)₂—.
385. The compound of any one of Embodiments 317-351, wherein L^{c} is —C(O)NHS(O)₂—.
386. The compound of any one of Embodiments 317-351, wherein L^{c} is —OC(O)N(R′)S(O)₂—.
387. The compound of any one of Embodiments 317-351, wherein L^{c} is —OC(O)NHS(O)₂—.
388. The compound of any one of Embodiments 317-351, wherein L^{c} is —N(R′)C(O)N(R′)S(O)₂—.
389. The compound of any one of Embodiments 317-351, wherein L^{c} is —NHC(O)NHS(O)₂—.
390. The compound of any one of Embodiments 376-389, wherein —C(O)—, —N(R′)—, —NH— or —S(O)₂— is bonded to R^{L}.
391. The compound of any one of Embodiments 376-389, wherein —C(O)—, —N(R′)—, —NH— or —S(O)₂— is bonded to L^{b}.
392. The compound of any one of Embodiments 317-351, wherein L^{c} is —C(O)N(R′)—.
393. The compound of any one of Embodiments 317-351, wherein L^{c} is —C(O)N(R′)S(O)₂—.
394. The compound of any one of Embodiments 317-351, wherein L^{c} is —C(O)N(R′)C(R′)₂C(O)N(R′)—.
395. The compound of any one of Embodiments 317-351, wherein L^{c} is —C(O)N(R′)C(R′)₂S(O)₂N(R′)—.
396. The compound of any one of Embodiments 317-351, wherein L^{c} is —C(O)N(R′)C(R′)₂P(O)(R′)—.
397. The compound of any one of Embodiments 317-351, wherein L^{c} is —C(O)N(R′)C(R′)₂N(R′)C(O)N(R′)S(O)₂—.
398. The compound of any one of Embodiments 317-351, wherein L^{c} is —C(O)N(R′)C(R′)₂—.
399. The compound of any one of Embodiments 317-351, wherein L^{c} is —C(O)N(R′)C(R′)₂C(O)N(R′)S(O)₂—.
400. The compound of any one of Embodiments 317-351, wherein L^{c} is —C(O)N(R′)S(O)₂N(R′)—.
401. The compound of any one of Embodiments 317-351, wherein L^{c} is —C(O)N(R′)C(NR′)N(R′)—.
402. The compound of any one of Embodiments 317-351, wherein L^{c} is —C(O)NHS(O)₂—.
403. The compound of any one of Embodiments 317-351, wherein L^{c} is —C(O)NHCH₂C(O)NH—.
404. The compound of any one of Embodiments 317-351, wherein L^{c} is —C(O)NHCH₂S(O)₂NH—.
405. The compound of any one of Embodiments 317-351, wherein L^{c} is —C(O)NHCH₂P(O)R′—.
406. The compound of any one of Embodiments 317-351, wherein L^{c} is —C(O)NHCH₂NHC(O)NHS(O)₂—.
407. The compound of any one of Embodiments 317-351, wherein L^{c} is —C(O)NHCH₂—.
408. The compound of any one of Embodiments 317-351, wherein L^{c} is —C(O)NHCH₂C(O)NHS(O)₂—.
409. The compound of any one of Embodiments 317-351, wherein L^{c} is —C(O)NHS(O)₂NH—.
410. The compound of any one of Embodiments 317-351, wherein L^{c} is —C(O)NHC(NR′)NH—.
411. The compound of any one of Embodiments 392-410, wherein —C(O)— is bonded to L^{b}.
412. The compound of any one of Embodiments 392-410, wherein —C(O)— is bonded to R^{L}.
413. The compound of any one of Embodiments 317-351, wherein L^{c} is —OS(O)₂O—.
414. The compound of any one of Embodiments 317-351, wherein L^{c} is —P(O)(R′)—.
415. The compound of any one of Embodiments 317-351, wherein L^{c} is —N(R′)C(O)N(R′)—.
416. The compound of any one of Embodiments 317-351, wherein L^{c} is —NHC(O)NH—.
417. The compound of any one of Embodiments 317-351, wherein L^{c} is —N(R′)C(NR′)—.
418. The compound of any one of Embodiments 317-351, wherein L^{c} is —NHC(NH)—.
419. The compound of any one of Embodiments 317-351, wherein L^{c} is —N(R′)C(NR′)N(R′)—.
420. The compound of any one of Embodiments 317-351, wherein L^{c} is —NHC(NH)NH—.
421. The compound of any one of Embodiments 317-351, wherein L^{c} is —N(R′)C(O)—.
422. The compound of any one of Embodiments 317-351, wherein L^{c} is —N(R′)C(S)—.
423. The compound of any one of Embodiments 317-351, wherein L^{c} is —N(R′)C(O)O—.
424. The compound of any one of Embodiments 317-351, wherein L^{c} is —N(R′)C(S)N(R′)S(O)₂—.
425. The compound of any one of Embodiments 317-351, wherein L^{c} is —N(R′)C(NR′)N(R′)S(O)₂—.
426. The compound of any one of Embodiments 317-351, wherein L^{c} is —N(R′)C(O)C(O)N(R′)S(O)₂—.
427. The compound of any one of Embodiments 421-426, wherein —N(R′)— is bonded to L^{b}.
428. The compound of any one of Embodiments 421-426, wherein —N(R′)— is bonded to R^{L}.
429. The compound of any one of Embodiments 317-351, wherein L^{c} is —NHC(O)—.
430. The compound of any one of Embodiments 317-351, wherein L^{c} is —NHC(S)—.
431. The compound of any one of Embodiments 317-351, wherein L^{c} is —NHC(O)O—.
432. The compound of any one of Embodiments 317-351, wherein L^{c} is —NHC(S)NHS(O)₂—.
433. The compound of any one of Embodiments 317-351, wherein L^{c} is —NHC(NH)NHS(O)₂—.
434. The compound of any one of Embodiments 317-351, wherein L^{c} is —NHC(O)C(O)NHS(O)₂—.
435. The compound of any one of Embodiments 429-434, wherein —NH— is bonded to L^{b}.
436. The compound of any one of Embodiments 429-434, wherein —NH— is bonded to R^{L}.
437. The compound of any one of Embodiments 317-351, wherein L^{c} is —N(R′)C(O)N(R′)S(O)₂N(R′)—.
438. The compound of 437, wherein —N(R′)C(O)— is bonded to L^{b}.
439. The compound of 437, wherein —N(R′)C(O)— is bonded to R^{L}.
440. The compound of any one of Embodiments 317-351, wherein L^{c} is —NHC(O)NHS(O)₂NH—.
441. The compound of Embodiment 440, wherein —NHC(O)— is bonded to L^{b}.
442. The compound of Embodiment 440, wherein —NHC(O)— is bonded to R^{L}.
443. The compound of any one of Embodiments 317-351, wherein L^{c} is —OC(O)N(R′)—.
444. The compound of any one of Embodiments 317-351, wherein L^{c} is —OC(O)N(R′)C(O)N(R′)—.
445. The compound of any one of Embodiments 317-351, wherein L^{c} is —OC(O)N(R′)C(O)—.
446. The compound of any one of Embodiments 317-351, wherein L^{c} is —OC(O)N(R′)C(O)N(R′)S(O)₂—.
447. The compound of any one of Embodiments 317-351, wherein L^{c} is —OC(O)N(R′)S(O)₂—.
448. The compound of any one of Embodiments 317-351, wherein L^{c} is —OC(O)NH—.
449. The compound of any one of Embodiments 317-351, wherein L^{c} is —OC(O)NHC(O)NH—.
450. The compound of any one of Embodiments 317-351, wherein L^{c} is —OC(O)NHC(O)—.
451. The compound of any one of Embodiments 317-351, wherein L^{c} is —OC(O)NHC(O)NHS(O)₂—.
452. The compound of any one of Embodiments 317-351, wherein L^{c} is —OC(O)NHS(O)₂—.
453. The compound of any one of Embodiments 443-452, wherein —O— is bonded to L^{b}.
454. The compound of any one of Embodiments 443-452, wherein —O— is bonded to R^{L}.
455. The compound of any one of Embodiments 317-351, wherein L^{c} is or comprises -Cy-.
456. The compound of any one of Embodiments 317-351, wherein L^{c} is -Cy-.
457. The compound of any one of Embodiments 455-456, wherein -Cy- is an optionally substituted 3-10 membered ring having 0-4 heteroatoms independently selected from nitrogen, oxygen and sulfur.
458. The compound of any one of Embodiments 455-456, wherein -Cy- is an optionally substituted 5-membered heteroaryl ring having 1-4 heteroatoms independently selected from nitrogen, oxygen and sulfur.
459. The compound of any one of Embodiments 455-456, wherein -Cy- is

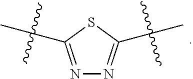 [SMILES: *c1nnc(*)s1]

460. The compound of any one of Embodiments 455-456, wherein -Cy- is optionally substituted

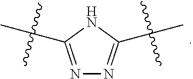 [SMILES: *c1nnc(*)[nH]1]

461. The compound of any one of Embodiments 455-456, wherein -Cy- is

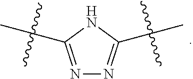 [SMILES: *c1nnc(*)[nH]1]

462. The compound of any one of Embodiments 1-191, wherein L¹ is —CH(CH₃)—(CH₂)m-, wherein m is 0-6.
463. The compound of any one of Embodiments 1-191, wherein L¹ is —(R)—CH(CH₃)—(CH₂)m-, wherein m is 0-6.
464. The compound of any one of Embodiments 1-191, wherein L¹ is —(S)—CH(CH₃)—(CH₂)m-, wherein m is 0-6.
465. The compound of any one of Embodiments 462-464, wherein m is 0.
466. The compound of any one of Embodiments 462-464, wherein m is 1.
467. The compound of any one of Embodiments 462-464, wherein m is 2.
468. The compound of any one of Embodiments 462-464, wherein m is 3.
469. The compound of any one of Embodiments 462-464, wherein m is 4.
470. The compound of any one of Embodiments 462-464, wherein m is 5.
471. The compound of any one of Embodiments 462-464, wherein m is 6.
472. The compound of any one of the preceding Embodiments, wherein R^{L} is R^{s}.
473. The compound of any one of Embodiments 1-471, wherein R^{L} is —C(O)OR^{s}.
474. The compound of any one of Embodiments 1-471, wherein R^{L} is —C(O)N(R^{s})₂.
475. The compound of any one of Embodiments 1-471, wherein R^{L} is —C(O)N(R′)S(O)₂R^{s}.
476. The compound of any one of Embodiments 1-471, wherein R^{L} is —C(O)N(R′)C(R′)₂C(O)N(R^{s})₂.
477. The compound of any one of Embodiments 1-471, wherein R^{L} is —C(O)N(R′)C(R′)₂S(O)₂R^{s}.
478. The compound of any one of Embodiments 1-471, wherein R^{L} is —C(O)N(R′)C(R′)₂S(O)₂N(R^{s})₂.
479. The compound of any one of Embodiments 1-471, wherein R^{L} is —C(O)N(R′)C(R′)₂P(O)(R^{s})₂.
480. The compound of any one of Embodiments 1-471, wherein R^{L} is —C(O)N(R′)C(R′)₂N(R′)C(O)N(R′)S(O)₂R^{s}.
481. The compound of any one of Embodiments 1-471, wherein R^{L} is —C(O)N(R′)C(R^{s})₃.
482. The compound of any one of Embodiments 1-471, wherein R^{L} is —C(O)N(R′)C(R′)₂C(O)N(R′)S(O)₂R^{t}.
483. The compound of any one of Embodiments 1-471, wherein R^{L} is —C(O)N(R′)S(O)₂N(R^{s})₂.
484. The compound of any one of Embodiments 1-471, wherein R^{L} is —C(O)N(R′)C(NR′)N(R^{s})₂.
485. The compound of any one of Embodiments 1-471, wherein R^{L} is —S(O)₂R^{s}.
486. The compound of any one of Embodiments 1-471, wherein R^{L} is S(O)₂N(R^{s})₂.
487. The compound of any one of Embodiments 1-471, wherein R^{L} is —P(O)(R^{s})₂.
488. The compound of any one of Embodiments 1-471, wherein R^{L} is —OS(O)₂R^{s}.
489. The compound of any one of Embodiments 1-471, wherein R^{L} 1S—OS(S)₂OR.
490. The compound of any one of Embodiments 1-471, wherein R^{L} is —N(R)₂.
491. The compound of any one of Embodiments 1-471, wherein R^{L} is —N(R′)C(O)R^{s}.
492. The compound of any one of Embodiments 1-471, wherein R^{L} is —N(R′)C(S)R^{s}.
493. The compound of any one of Embodiments 1-471, wherein R^{L} is —N(R′)C(NR′)R^{s}.
494. The compound of any one of Embodiments 1-471, wherein R^{L} is —N(R′)C(O)OR^{s}.
495. The compound of any one of Embodiments 1-471, wherein R^{L} is —N(R′)C(O)N(R)₂.
496. The compound of any one of Embodiments 1-471, wherein R^{L} is —N(R′)C(NR′)N(R)₂.
497. The compound of any one of Embodiments 1-471, wherein R^{L} is —N(R′)C(O)N(R′)S(O)₂R^{s}.
498. The compound of any one of Embodiments 1-471, wherein R^{L} is —N(R′)C(S)N(R′)S(O)₂R^{s}.
499. The compound of any one of Embodiments 1-471, wherein R^{L} is —N(R′)C(NR′)N(R′)S(O)₂R^{s}.
500. The compound of any one of Embodiments 1-471, wherein R^{L} is —N(R′)C(O)C(O)N(R′)S(O)₂R.
501. The compound of any one of Embodiments 1-471, wherein R^{L} is —N(R′)C(O)N(R′)S(O)₂N(R)₂.
502. The compound of any one of Embodiments 1-471, wherein R^{L} is —N(R′)S(O)₂R.
503. The compound of any one of Embodiments 1-471, wherein R^{L} is —OC(O)N(R^{s})₂.
504. The compound of any one of Embodiments 1-471, wherein R^{L} is —OC(O)N(R′)C(O)N(R^{s})₂.
505. The compound of any one of Embodiments 1-471, wherein R^{L} is OC(O)N(R′)C(O)R.
506. The compound of any one of Embodiments 1-471, wherein R^{L} i —OC(O)N(R′)C(O)N(R′)S(O)₂R^{s}.
507. The compound of any one of Embodiments 1-471, wherein R^{L} is —OC(O)N(R′)S(O)₂R^{s}.
508. The compound of any one of Embodiments 1-471, wherein R^{L} is —C(O)R^{s}.
509. The compound of any one of Embodiments 1-471, wherein R^{L} is —C(O)OR^{s}.
510. The compound of any one of Embodiments 1-471, wherein R^{L} is —C(O)N(R^{s})₂.
511. The compound of any one of Embodiments 1-471, wherein R^{L} is —C(O)NHS(O)₂R^{s}.
512. The compound of any one of Embodiments 1-471, wherein R^{L} is —C(O)NHCH₂C(O)N(R^{s})₂.
513. The compound of any one of Embodiments 1-471, wherein R^{L} is —C(O)NHCH₂S(O)₂R^{s}.
514. The compound of any one of Embodiments 1-471, wherein R^{L} is —C(O)NHCH₂S(O)₂N(R^{s})₂.
515. The compound of any one of Embodiments 1-471, wherein R^{L} is —C(O)NHCH₂P(O)(R^{s})₂.
516. The compound of any one of Embodiments 1-471, wherein R^{L} is —C(O)NHCH₂NHC(O)NHS(O)₂R^{s}.
517. The compound of any one of Embodiments 1-471, wherein R^{L} is —C(O)NHC(R^{s})₃.
518. The compound of any one of Embodiments 1-471, wherein R^{L} is —C(O)NHCH₂C(O)NHS(O)₂R^{s}.
519. The compound of any one of Embodiments 1-471, wherein R^{L} is —C(O)NHS(O)₂N(R^{s})₂.
520. The compound of any one of Embodiments 1-471, wherein R^{L} is —C(O)NHC(NH)N(R^{s})₂.
521. The compound of any one of Embodiments 1-471, wherein R^{L} is —S(O)₂R^{s}.
522. The compound of any one of Embodiments 1-471, wherein R^{L} is —S(O)₂N(R^{s})₂.
523. The compound of any one of Embodiments 1-471, wherein R^{L} is —P(O)(R^{s})₂.
524. The compound of any one of Embodiments 1-471, wherein R^{L} is —OS(O)₂R^{s}.
525. The compound of any one of Embodiments 1-471, wherein R^{L} is —OS(O)₂OR^{s}.
526. The compound of any one of Embodiments 1-471, wherein R^{L} is —N(R^{s})₂.
527. The compound of any one of Embodiments 1-471, wherein R^{L} is —NHC(O)R^{s}.
528. The compound of any one of Embodiments 1-471, wherein R^{L} is —NHC(S)R^{s}.
529. The compound of any one of Embodiments 1-471, wherein R^{L} is —NHC(NH)R^{s}.
530. The compound of any one of Embodiments 1-471, wherein R^{L} is —NHC(O)OR^{s}.
531. The compound of any one of Embodiments 1-471, wherein R^{L} is —NHC(O)N(R^{s})₂.
532. The compound of any one of Embodiments 1-471, wherein R^{L} is —NHC(NH)N(R^{s})₂.
533. The compound of any one of Embodiments 1-471, wherein R^{L} is —NHC(O)NHS(O)₂R^{s}.
534. The compound of any one of Embodiments 1-471, wherein R^{L} is —NHC(S)NHS(O)₂R^{s}.
535. The compound of any one of Embodiments 1-471, wherein R^{L} is —NHC(NH)NHS(O)₂R^{s}.
536. The compound of any one of Embodiments 1-471, wherein R^{L} is —NHC(O)C(O)NHS(O)₂R^{s}.
537. The compound of any one of Embodiments 1-471, wherein R^{L} is —NHC(O)NHS(O)₂N(R^{s})₂.
538. The compound of any one of Embodiments 1-471, wherein R^{L} is —NHS(O)₂R^{s}.
539. The compound of any one of Embodiments 1-471, wherein R^{L} is —OC(O)N(R^{s})₂.
540. The compound of any one of Embodiments 1-471, wherein R^{L} is —OC(O)NHC(O)N(R^{s})₂.
541. The compound of any one of Embodiments 1-471, wherein R^{L} is —OC(O)NHC(O)R^{s}.
542. The compound of any one of Embodiments 1-471, wherein R^{L} is —OC(O)NHC(O)NHS(O)₂R^{s}.
543. The compound of any one of Embodiments 1-471, wherein R^{L} is —OC(O)NHS(O)₂R^{s}.
544. The compound of any one of Embodiments 472-543, wherein R^{s} is —H.
545. The compound of any one of Embodiments 472-543, wherein R^{s} is halogen.
546. The compound of any one of Embodiments 472-543, wherein R^{s} is —CN.
547. The compound of any one of Embodiments 472-543, wherein R^{s} is —N₃.
548. The compound of any one of Embodiments 472-543, wherein R^{s} is —OR′.
549. The compound of any one of Embodiments 472-543, wherein R^{s} is —C(O)R′.
550. The compound of any one of Embodiments 472-543, wherein R^{s} is —S(O)₂R′.
551. The compound of any one of Embodiments 472-543, wherein R^{s} is —S(O)₂N(R′)₂.
552. The compound of any one of Embodiments 472-543, wherein R^{s} is —SO₃R′.
553. The compound of any one of Embodiments 472-543, wherein R^{s} is —OS(O)₂R′.
554. The compound of any one of Embodiments 472-543, wherein R^{s} is —OP(O)(R′)₂.
555. The compound of any one of Embodiments 472-543, wherein R^{s} is —OP(O)(OR′)₂.
556. The compound of any one of Embodiments 472-543, wherein R^{s} is —P(O)(R′)₂.
557. The compound of any one of Embodiments 472-543, wherein R^{s} is —PO(OR′)₂.
558. The compound of any one of Embodiments 472-543, wherein R^{s} is —SR′.
559. The compound of any one of Embodiments 472-543, wherein R^{s} is —C(O)N(R′)₂.
560. The compound of any one of Embodiments 472-543, wherein R^{s} is —N(R′)₂.
561. The compound of any one of Embodiments 472-543, wherein R^{s} is a protected hydroxyl group.
562. The compound of any one of Embodiments 472-543, wherein R^{s} is R^{s} is

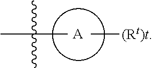

563. The compound of any one of Embodiments 472-543, wherein two R^{s} attached to the same atom are taken together to form ═O or ═NR^{x}.
564. The compound of any one of Embodiments 472-543, wherein R^{s} is -L″-R′.
565. The compound of Embodiment 564, wherein R′ is —H.
566. The compound of Embodiment 564, wherein R′ is optionally substituted C₁₋₁₀ aliphatic.
567. The compound of Embodiment 566, wherein R′ is optionally substituted C₁₋₈ alkyl.
568. The compound of Embodiment 566, wherein R′ is optionally substituted C₂₋₈ alkenyl.
569. The compound of Embodiment 566, wherein R′ is optionally substituted C₂₋₈ alkynyl.
570. The compound of Embodiment 566, wherein R′ is optionally substituted C₃₋₈ cycloalkyl.
571. The compound of Embodiment 567, wherein R′ is C₁₋₄ alkyl
572. The compound of Embodiment 567, wherein R′ is methyl.
573. The compound of Embodiment 567, wherein R′ is ethyl.
574. The compound of Embodiment 567, wherein R′ is t-butyl.
575. The compound of Embodiment 567, wherein R′ is 2-hydroxyl-t-butyl.
576. The compound of Embodiment 567, wherein R′ is cyclohexyl.
577. The compound of Embodiment 566, wherein R′ is

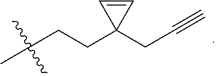 [SMILES: *CCC1(CC#C)C=C1]

578. The compound of Embodiment 564, wherein R′ is optionally substituted C₆₋₁₀ aryl.
579. The compound of Embodiment 564, wherein R′ is optionally substituted phenyl.
580. The compound of Embodiment 564, wherein R′ is optionally substituted C₆₋₁₅ arylaliphatic.
581. The compound of Embodiment 580, wherein R′ is optionally substituted C₆₋₁₅ arylalkyl.
582. The compound of Embodiment 564, wherein R′ is 4-tert-butylphenyl.
583. The compound of Embodiment 564, wherein R′ is 4-(2-hydroxyl-1-dimethylethyl)phenyl.
584. The compound of Embodiment 564, wherein R′ is optionally substituted 3-12 membered heterocyclyl having 1-5 heteroatoms.
585. The compound of Embodiment 564, wherein R′ is optionally substituted 5-14 membered heteroaryl having 1-10 heteroatoms.
586. The compound of Embodiment 564, wherein R′ is optionally substituted C₆₋₁₅ heteroarylaliphatic having 1-5 heteroatoms.
587. The compound of any one of Embodiments 564-586, wherein L″ is -L^{a}″-L^{b}″-L^{c}″-, wherein:
   L^{a}″ is a covalent bond, or an optionally substituted, bivalent C₁₋₃ aliphatic group wherein one or more methylene units of the group are independently and optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
   L^{b}″ is a covalent bond, or an optionally substituted, bivalent C₁₋₂ aliphatic group wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—; and
   L^{c}″ is a covalent bond, or an optionally substituted, bivalent C₁ aliphatic group wherein a methylene unit of the group is optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —N(R′)C(O)N(R′)—, —N(R′)C(NR′)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—.
588. The compound of Embodiment 587, wherein L^{a}″ is bonded to R′.
589. The compound of Embodiment 587, wherein L^{c}″ is bonded to R′.
590. The compound of any one of Embodiments 587-589, wherein L^{a}″ is a covalent bond.
591. The compound of any one of Embodiments 587-589, wherein L^{a}″ is optionally substituted bivalent C₁₋₁₀ aliphatic.
592. The compound of any one of Embodiments 587-589, wherein L^{a}″ is C₁₋₁₀ is optionally substituted alkylene.
593. The compound of any one of Embodiments 587-589, wherein L^{a}″ is optionally substituted —(CH₂)₁₋₁₀—.
594. The compound of any one of Embodiments 587-589, wherein L^{a}″ is optionally substituted —CH₂—.
595. The compound of any one of Embodiments 587-589, wherein L^{a}″ is optionally substituted —(CH₂)₂—.
596. The compound of any one of Embodiments 587-589, wherein L^{a}″ is optionally substituted —(CH₂)₃—.
597. The compound of any one of Embodiments 587-589, wherein L^{a}″ is —(CH₂)₁₋₁₀—.
598. The compound of any one of Embodiments 587-589, wherein L^{a}″ is —CH₂—.
599. The compound of any one of Embodiments 587-589, wherein L^{a}″ is —(CH₂)₂—.
600. The compound of any one of Embodiments 587-589, wherein L^{a}″ is —(CH₂)₃—.
601. The compound of any one of Embodiments 587-589, wherein L^{a}″ is —CH(COOH)—.
602. The compound of any one of Embodiments 587-589, wherein L^{a}″ is —CH(CN)—.
603. The compound of any one of Embodiments 587-589, wherein L^{a}″ is —C(R′)₂—.
604. The compound of any one of Embodiments 587-589, wherein L^{a}″ is —CHR′—.
605. The compound of any one of Embodiments 587-589, wherein L^{a}″ is —CH(CH₃)—.
606. The compound of any one of Embodiments 587-589, wherein L^{a}″ is —(R)—CH(CH₃)—.
607. The compound of any one of Embodiments 587-589, wherein L^{a}″ is —(S)—CH(CH₃)—.
608. The compound of any one of Embodiments 587-589, wherein L^{a}″ is -L″-.
609. The compound of any one of Embodiments 587-589, wherein L^{a}″ is —O—.
610. The compound of any one of Embodiments 587-589, wherein L^{a}″ is —C(O)—.
611. The compound of any one of Embodiments 587-589, wherein L^{a}″ is —S(O)₂—.
612. The compound of any one of Embodiments 587-589, wherein L^{a}″ is —N(R′)—.
613. The compound of any one of Embodiments 587-589, wherein L^{a}″ is —NH—.
614. The compound of any one of Embodiments 587-589, wherein L^{a}″ is —OC(O)—.
615. The compound of any one of Embodiments 587-589, wherein L^{a}″ is —OC(O)N(R′)—.
616. The compound of any one of Embodiments 587-589, wherein L^{a}″ is —OC(O)NH—.
617. The compound of any one of Embodiments 587-589, wherein L^{a}″ is —OC(O)N(R′)S(O)₂—.
618. The compound of any one of Embodiments 587-589, wherein L^{a}″ is —OC(O)NHS(O)₂—.
619. The compound of any one of Embodiments 587-589, wherein L^{a}″ is —OS(O)₂—.
620. The compound of any one of Embodiments 587-589,
621. The compound of any one of Embodiments 587-589, wherein L^{a}″ is —NHS(O)₂—.
622. The compound of any one of Embodiments 587-589, wherein L^{a}″ is —C(O)N(R′)S(O)₂—.
623. The compound of any one of Embodiments 587-589, wherein L^{a}″ is —C(O)NHS(O)₂—.
624. The compound of any one of Embodiments 587-589, wherein L^{a}″ is —OC(O)N(R′)S(O)₂—.
625. The compound of any one of Embodiments 587-589, wherein L^{a}″ is —OC(O)NHS(O)₂—.
626. The compound of any one of Embodiments 587-589, wherein L^{a}″ is —N(R′)C(O)N(R′)S(O)₂—.
627. The compound of any one of Embodiments 587-589, wherein L^{a}″ is —NHC(O)NHS(O)₂—.
628. The compound of any one of Embodiments 614-627, wherein —C(O)—, —N(R′)—, —NH— or —S(O)₂— is bonded to L¹.
629. The compound of any one of Embodiments 614-627, wherein —C(O)—, —N(R′)—, —NH— or —S(O)₂— is bonded to L″.
630. The compound of any one of Embodiments 587-589, wherein L^{a}″ is —C(O)N(R′)—.
631. The compound of any one of Embodiments 587-589, wherein L^{a}″ is —C(O)N(R′)S(O)₂—.
632. The compound of any one of Embodiments 587-589, wherein L^{a}″ is —C(O)N(R′)C(R′)₂C(O)N(R′)—.
633. The compound of any one of Embodiments 587-589, wherein L^{a}″ is —C(O)N(R′)C(R′)₂S(O)₂N(R′)—.
634. The compound of any one of Embodiments 587-589, wherein L^{a}″ is —C(O)N(R′)C(R′)₂P(O)(R′)—.
635. The compound of any one of Embodiments 587-589, wherein L^{a}″ is —C(O)N(R′)C(R′)₂N(R′)C(O)N(R′)S(O)₂—.
636. The compound of any one of Embodiments 587-589, wherein L^{a}″ is —C(O)N(R′)C(R′)₂—.
637. The compound of any one of Embodiments 587-589, wherein L^{a}″ is —C(O)N(R′)C(R′)₂C(O)N(R′)S(O)₂—.
638. The compound of any one of Embodiments 587-589, wherein L^{a}″ is —C(O)N(R′)S(O)₂N(R′)—.
639. The compound of any one of Embodiments 587-589, wherein L^{a}″ is —C(O)N(R′)C(NR′)N(R′)—.
640. The compound of any one of Embodiments 587-589, wherein L^{a}″ is —C(O)NHS(O)₂—.
641. The compound of any one of Embodiments 587-589, wherein L^{a}″ is —C(O)NHCH₂C(O)NH—.
642. The compound of any one of Embodiments 587-589,
643. The compound of any one of Embodiments 587-589, wherein L^{a}″ is —C(O)NHCH₂P(O)R′—.
644. The compound of any one of Embodiments 587-589, wherein L^{a}″ is —C(O)NHCH₂NHC(O)NHS(O)₂—.
645. The compound of any one of Embodiments 587-589, wherein L^{a}″ is —C(O)NHCH₂—.
646. The compound of any one of Embodiments 587-589, wherein L^{a}″ is —C(O)NHCH₂C(O)NHS(O)₂—.
647. The compound of any one of Embodiments 587-589, wherein L^{a}″ is —C(O)NHS(O)₂NH—.
648. The compound of any one of Embodiments 587-589, wherein L^{a}″ is —C(O)NHC(NR′)NH—.
649. The compound of any one of Embodiments 630-648, wherein —C(O)— is bonded to L¹.
650. The compound of any one of Embodiments 630-648, wherein —C(O)— is bonded to L^{b}′.
651. The compound of any one of Embodiments 587-589, wherein L^{a}″ is —OS(O)₂O—.
652. The compound of any one of Embodiments 587-589, wherein L^{a}″ is —P(O)(R′)—.
653. The compound of any one of Embodiments 587-589, wherein L^{a}″ is —N(R′)C(O)N(R′)—.
654. The compound of any one of Embodiments 587-589, wherein L^{a}″ is —NHC(O)NH—.
655. The compound of any one of Embodiments 587-589, wherein L^{a}″ is —N(R′)C(NR′)—.
656. The compound of any one of Embodiments 587-589, wherein L^{a}″ is —NHC(NH)—.
657. The compound of any one of Embodiments 587-589, wherein L^{a}″ is —N(R′)C(NR′)N(R′)—.
658. The compound of any one of Embodiments 587-589, wherein L^{a}″ is —NHC(NH)NH—.
659. The compound of any one of Embodiments 587-589, wherein L^{a}″ is —N(R′)C(O)—.
660. The compound of any one of Embodiments 587-589, wherein L^{a}″ is —N(R′)C(S)—.
661. The compound of any one of Embodiments 587-589, wherein L^{a}″ is —N(R′)C(O)O—.
662. The compound of any one of Embodiments 587-589, wherein L^{a}″ is —N(R′)C(S)N(R′)S(O)₂—.
663. The compound of any one of Embodiments 587-589, wherein L^{a}″ is —N(R′)C(NR′)N(R′)S(O)₂—.
664. The compound of any one of Embodiments 587-589, wherein L^{a}″ is —N(R′)C(O)C(O)N(R′)S(O)₂—.
665. The compound of any one of Embodiments 659-664, wherein —N(R′)— is bonded to L¹.
666. The compound of any one of Embodiments 659-664, wherein —N(R′)— is bonded to L″.
667. The compound of any one of Embodiments 587-589, wherein L^{a}″ is —NHC(O)—.
668. The compound of any one of Embodiments 587-589, wherein L^{a}″ is —NHC(S)—.
669. The compound of any one of Embodiments 587-589, wherein L^{a}″ is —NHC(O)O—.
670. The compound of any one of Embodiments 587-589, wherein L^{a}″ is —NHC(S)NHS(O)₂—.
671. The compound of any one of Embodiments 587-589,
672. The compound of any one of Embodiments 587-589, wherein L^{a}″ is —NHC(O)C(O)NHS(O)₂—.
673. The compound of any one of Embodiments 667-672, wherein —NH— is bonded to L¹.
674. The compound of any one of Embodiments 667-672, wherein —NH— is bonded to L″.
675. The compound of any one of Embodiments 587-589, wherein L^{a}″ is —N(R′)C(O)N(R′)S(O)₂N(R′)—.
676. The compound of 675, wherein —N(R′)C(O)— is bonded to L^{b}.
677. The compound of 675, wherein —N(R′)C(O)— is bonded to R^{L}.
678. The compound of any one of Embodiments 587-589, wherein L^{a}″ is —NHC(O)NHS(O)₂NH—.
679. The compound of Embodiment 678, wherein —NHC(O)— is bonded to L^{b}.
680. The compound of Embodiment 678, wherein —NHC(O)— is bonded to R^{L}.
681. The compound of any one of Embodiments 587-589, wherein L^{a}″ is —OC(O)N(R′)—.
682. The compound of any one of Embodiments 587-589, wherein L^{a}″ is —OC(O)N(R′)C(O)N(R′)—.
683. The compound of any one of Embodiments 587-589, wherein L^{a}″ is —OC(O)N(R′)C(O)—.
684. The compound of any one of Embodiments 587-589, wherein L^{a}″ is —OC(O)N(R′)C(O)N(R′)S(O)₂—.
685. The compound of any one of Embodiments 587-589, wherein L^{a}″ is —OC(O)N(R′)S(O)₂—.
686. The compound of any one of Embodiments 587-589, wherein L^{a}″ is —OC(O)NH—.
687. The compound of any one of Embodiments 587-589, wherein L^{a}″ is —OC(O)NHC(O)NH—.
688. The compound of any one of Embodiments 587-589, wherein L^{a}″ is —OC(O)NHC(O)—.
689. The compound of any one of Embodiments 587-589, wherein L^{a}″ is —OC(O)NHC(O)NHS(O)₂—.
690. The compound of any one of Embodiments 587-589, wherein L^{a}″ is —OC(O)NHS(O)₂—.
691. The compound of any one of Embodiments 681-690, wherein —O— is bonded to L^{b}.
692. The compound of any one of Embodiments 681-690, wherein —O— is bonded to R^{L}.
693. The compound of any one of Embodiments 587-589, wherein L^{a}″ is or comprises -Cy-.
694. The compound of any one of Embodiments 587-589, wherein L^{a}″ is -Cy-.
695. The compound of any one of Embodiments 587-694, wherein L^{b}″ is a covalent bond.
696. The compound of any one of Embodiments 587-694, wherein L^{b}″ is optionally substituted —CH₂—.
697. The compound of any one of Embodiments 587-694, wherein L^{b}″ is optionally substituted —CH₂—CH₂—.
698. The compound of any one of Embodiments 587-694, wherein L^{b}″ is —C(R′)₂—.
699. The compound of any one of Embodiments 587-694, wherein L^{b}″ is —C(R′)₂—C(R′)₂—.
700. The compound of any one of Embodiments 698-699, wherein two R′ of —C(R′)₂— are taken together with the carbon atom to which they are attached to form an optionally substituted ring.
701. The compound of any one of Embodiments 587-694, wherein L^{b}″ is —C(O)—.
702. The compound of any one of Embodiments 587-694, wherein L^{b}″ is —N(R′)—.
703. The compound of any one of Embodiments 587-694, wherein L^{b}″ is —NH—.
704. The compound of any one of Embodiments 587-694, wherein L^{b}″ is —S(O)₂—.
705. The compound of any one of Embodiments 587-694, wherein L^{b}″ is —S(O)₂N(R′)—.
706. The compound of any one of Embodiments 587-694, wherein L^{b}″ is —S(O)₂NH—.
707. The compound of any one of Embodiments 587-694, wherein L^{b}″ is or comprises -Cy-.
708. The compound of any one of Embodiments 587-694, wherein L^{b}″ is -Cy-.
709. The compound of any one of Embodiments 587-694, wherein L^{b}″ is -Cy-CH₂— and the —CH₂— is optionally substituted.
710. The compound of any one of Embodiments 587-694, wherein L^{b}″ is -Cy-C(R′)₂—.
711. The compound of any one of Embodiments 587-694, wherein L^{b}″ is -Cy-CHR—.
712. The compound of any one of Embodiments 709-711, wherein -Cy- is bonded to L^{a}″.
713. The compound of any one of Embodiments 709-711, wherein -Cy- is bonded to L^{c}″.
714. The compound of any one of Embodiments 587-713, wherein L^{c}″ is a covalent bond.
715. The compound of any one of Embodiments 587-713, wherein L^{c}″ is optionally substituted —CH₂—.
716. The compound of any one of Embodiments 587-713, wherein L^{c}″ is —C(R′)₂—.
717. The compound of any one of Embodiments 587-713, wherein L^{c}″ is —CHR—.
718. The compound of any one of Embodiments 587-713, wherein L^{c}″ is —O—.
719. The compound of any one of Embodiments 587-713, wherein L^{c}″ is —C(O)—.
720. The compound of any one of Embodiments 587-713, wherein L^{c}″ is —C(O)O—.
721. The compound of any one of Embodiments 587-713, wherein L^{c}″ is —N(R′)—.
722. The compound of any one of Embodiments 587-713, wherein L^{c}″ is —NH—.
723. The compound of any one of Embodiments 587-713, wherein L^{c}″ is —C(O)N(R′)—.
724. The compound of any one of Embodiments 587-713, wherein L^{c}″ is —C(O)N(R′)—.
725. The compound of any one of Embodiments 587-713, wherein L^{c}″ is —C(O)NH—.
726. The compound of any one of Embodiments 587-713, wherein L^{c}″ is —C(O)N(CH₃)—.
727. The compound of any one of Embodiments 587-713, wherein L^{c}″ is —S(O)₂—.
728. The compound of any one of Embodiments 587-713, wherein L^{c}″ is -Cy-.
729. The compound of any one of the preceding Embodiments, wherein -Cy- is optionally substituted 4-20 membered, monocyclic, bicyclic or polycyclic ring having 0-10 heteroatoms.
730. The compound of any one of Embodiments 1-729, wherein -Cy- is has 1-10 (1, 2, 3, 4, 5, 6, 7, 8, 9, or 10) heteroatoms.
731. The compound of any one of Embodiments 1-729, wherein -Cy- is has no heteroatoms.
732. The compound of any one of the preceding Embodiments, wherein -Cy- is 3-10 membered.
733. The compound of any one of the preceding Embodiments, wherein -Cy- is 5-10 membered.
734. The compound of any one of the preceding Embodiments, wherein -Cy- is monocyclic.
735. The compound of any one of the preceding Embodiments, wherein -Cy- is bicyclic.
736. The compound of any one of the preceding Embodiments, wherein -Cy- is polycyclic.
737. The compound of any one of the preceding Embodiments, wherein each monocyclic unit is independently 3-10 (3, 4, 5, 6, 7, 8, 9, or 10) membered, is independently saturated, partially unsaturated or aromatic, and independently has 0-4 (e.g., 0, 1, 2, 3, or 4) heteroatoms.
738. The compound of any one of the preceding Embodiments, wherein a monocyclic unit is a phenyl ring.
739. The compound of any one of the preceding Embodiments, wherein a monocyclic unit is a 5-membered heteroaryl having 1-4 heteroatoms.
740. The compound of any one of the preceding Embodiments, wherein a monocyclic unit is a 6-membered heteroaryl having 1-4 heteroatoms.
741. The compound of any one of the preceding Embodiments, wherein a monocyclic ring is a saturated or partially unsaturated C₃₋₇ cycloaliphatic.
742. The compound of any one of the preceding Embodiments, wherein a monocyclic ring is a saturated or partially unsaturated 3-membered heterocyclyl having 1-4 heteroatoms.
743. The compound of any one of Embodiments 1-728, wherein -Cy- is optionally substituted

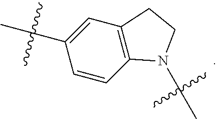 [SMILES: *c1ccc2c(c1)CCN2*]

744. The compound of any one of Embodiments 1-728, wherein -Cy- is optionally substituted

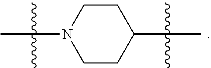 [SMILES: *C1CCN(*)CC1]

745. The compound of any one of Embodiments 1-728, wherein -Cy- is

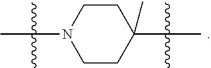 [SMILES: *N1CCC(*)(C)CC1]

746. The compound of any one of Embodiments 1-728, wherein -Cy- is optionally substituted

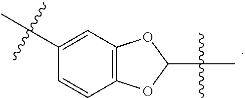 [SMILES: *c1ccc2c(c1)OC(*)O2]

747. The compound of any one of Embodiments 1-728, wherein -Cy- is optionally substituted

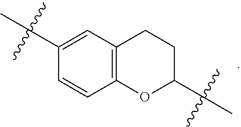 [SMILES: *c1ccc2c(c1)CCC(*)O2]

748. The compound of any one of Embodiments 1-728, wherein -Cy- is an optionally substituted 3-10 membered ring having 0-4 heteroatoms independently selected from nitrogen, oxygen and sulfur.
749. The compound of any one of Embodiments 1-728, wherein -Cy- is an optionally substituted 5-membered heteroaryl ring having 1-4 heteroatoms independently selected from nitrogen, oxygen and sulfur.
750. The compound of any one of Embodiments 1-728, wherein -Cy- is

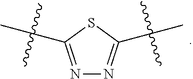 [SMILES: *c1nnc(*)s1]

751. The compound of any one of Embodiments 1-728, wherein -Cy- is optionally substituted

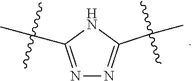 [SMILES: *c1nnc(*)[nH]1]

752. The compound of any one of Embodiments 1-728, wherein -Cy- is

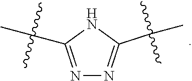 [SMILES: *c1nnc(*)[nH]1]

753. The compound of any one of Embodiments 1-728, wherein -Cy- is optionally substituted

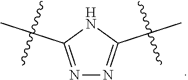 [SMILES: *c1nnc(*)[nH]1]

754. The compound of any one of Embodiments 1-728, wherein -Cy- is optionally substituted

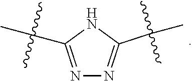 [SMILES: *c1nnc(*)[nH]1]

755. The compound of any one of Embodiments 1-728, wherein -Cy- is optionally substituted

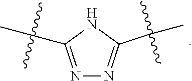 [SMILES: *c1nnc(*)[nH]1]

756. The compound of any one of Embodiments 1-316, wherein R^{L} is —N(R′)₂.
757. The compound of Embodiment 588, wherein R^{L} is —N(R′)₂ wherein each R′ is independently —H or optionally substituted C₁₋₆ aliphatic.
758. The compound of Embodiment 588, wherein R^{L} is —N(R′)₂ wherein the two R′ are taken together with the nitrogen atom to which they are attached to form an optionally substituted 3-20 membered ring having, in addition to the nitrogen atom, 0-4 heteroatoms.
759. The compound of Embodiment 758, wherein the formed ring is saturated.
760. The compound of any one of Embodiments 1-316, wherein R^{L} is —C(O)R.
761. The compound of Embodiment 760, wherein R^{L} is —C(O)R wherein R is —H or optionally substituted C₁₋₆ aliphatic.
762. The compound of Embodiment 761, wherein R^{L} is —C(O)CH₃.
763. The compound of any one of Embodiments 1-316, wherein R^{L} is —C(O)OR.
764. The compound of Embodiment 763, wherein R^{L} is —C(O)OR wherein R is —H or optionally substituted C₁₋₆ aliphatic.
765. The compound of Embodiment 764, wherein R^{L} is —COOH.
766. The compound of Embodiment 764, wherein R^{L} is —COOCH₃.
767. The compound of Embodiment 764, wherein R^{L} is -COOEt.
768. The compound of any one of Embodiments 1-316, wherein R^{L} is halogen.
769. The compound of any one of Embodiments 1-316, wherein R^{L} is —CN.
770. The compound of any one of Embodiments 1-316, wherein R^{L} is —OR.
771. The compound of Embodiment 770, wherein R^{L} is —OH.
772. The compound of any one of Embodiments 1-316, wherein R^{L} is —OS(O)₂OR.
773. The compound of Embodiment 772, wherein R^{L} is —OS(O)₂OH.
774. The compound of any one of Embodiments 1-316, wherein R^{L} is N(R′).
775. The compound of Embodiment 774, wherein R^{L} is —NHR wherein R is —H or optionally substituted C₁₋₆ aliphatic.
776. The compound of Embodiment 775, wherein R^{L} is -NHtBu.
777. The compound of any one of Embodiments 1-316, wherein R^{L} is —C(O)N(R′)S(O)₂R^{s}.
778. The compound of any one of Embodiments 1-316, wherein R^{L} is —C(O)N(R′)CH(R′)C(O)N(R′)S(O)₂R^{s}.
779. The compound of any one of Embodiments 1-316, wherein R^{L} is —C(O)N(R′)C(NR′)N(R^{s})₂.
780. The compound of any one of Embodiments 1-316, wherein R^{L} is -L₃₂-R^{s}.
781. The compound of Embodiment 780, wherein L₃₂ is a covalent bond.
782. The compound of Embodiment 780, wherein R^{L} is —C(O)N(R′)—R^{s}.
783. The compound of Embodiment 780, wherein R^{L} is —C(O)NH—R^{s}.
784. The compound of Embodiment 780, wherein R^{L} is —N(R′)—R^{s}.
785. The compound of Embodiment 780, wherein R^{L} is —NH—R^{s}.
786. The compound of any one of Embodiments 780-785, wherein R^{s} is optionally substituted heteroaryl.
787. The compound of any one of Embodiments 780-785, wherein R^{s} is optionally substituted 5-membered heteroaryl having 1-4 heteroatom.
788. The compound of any one of Embodiments 780-785, wherein R^{s} is optionally substituted tetrazolyl.
789. The compound of any one of the preceding Embodiments, wherein each R is independently —H, or an optionally substituted group selected from C₁₋₁₀ aliphatic, C₁₋₁₀ heteroaliphatic having 1-5 heteroatoms, C₆₋₁₄ aryl, C₆₋₁₄ aryl-C₁₋₁₀ aliphatic, C₆₋₁₄ aryl-C₁₋₁₀ heteroaliphatic having 1-5 heteroatoms, C₁₋₁₀ aliphatic-C₆₋₁₄ aryl, C₁₋₁₀ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₀ aliphatic, 5-14 membered heteroaryl having 1-10 heteroatoms-C₁₋₁₀ heteroaliphatic having 1-5 heteroatoms, C₁₋₁₀ aliphatic-5-14 membered heteroaryl having 1-10 heteroatoms, C₁₋₁₀ heteroaliphatic having 1-5 heteroatoms-5-14 membered heteroaryl having 1-10 heteroatoms, C₂-C₂₀ biaryl having 0-10 heteroatoms wherein each aromatic unit is independently 5-10 membered and has 0-10 heteroatoms, and 3-15 membered heterocyclyl having 1-5 heteroatoms, or
   two R groups are optionally and independently taken together to form a covalent bond or ═O, or:
   two or more R groups on the same atom are optionally and independently taken together with the atom to form an optionally substituted, 3-15 membered, monocyclic, bicyclic or polycyclic ring having, in addition to the atom, 0-5 heteroatoms; or
   two or more R groups on two or more atoms are optionally and independently taken together with their intervening atoms to form an optionally substituted, 3-15 membered, monocyclic, bicyclic or polycyclic ring having, in addition to the intervening atoms, 0-5 heteroatoms.
790. The compound of any one of the preceding Embodiments, wherein each heteroatom is independently selected from nitrogen, oxygen, sulfur, silicon and phosphorus.
791. The compound of any one of the preceding Embodiments, wherein each heteroatom is independently selected from nitrogen, oxygen and sulfur.
792. The compound of any one of the preceding Embodiments, wherein a formed ring is optionally substituted 4-20 membered, monocyclic, bicyclic or polycyclic ring having 0-10 heteroatoms.
793. The compound of any one of Embodiments 1-791, wherein a formed ring has 1-10 (1, 2, 3, 4, 5, 6, 7, 8, 9, or 10) heteroatoms.
794. The compound of any one of Embodiments 1-791, wherein a formed ring has no heteroatoms.
795. The compound of any one of the preceding Embodiments, wherein a formed ring is 3-10 membered.
796. The compound of any one of the preceding Embodiments, wherein a formed ring is 5-10 membered.
797. The compound of any one of the preceding Embodiments, wherein a formed ring is monocyclic.
798. The compound of any one of the preceding Embodiments, wherein a formed ring is bicyclic.
799. The compound of any one of the preceding Embodiments, wherein a formed ring is polycyclic.
800. The compound of any one of the preceding Embodiments, wherein each monocyclic unit is independently 3-10 (3, 4, 5, 6, 7, 8, 9, or 10) membered, is independently saturated, partially unsaturated or aromatic, and independently has 0-4 (e.g., 0, 1, 2, 3, or 4) heteroatoms.
801. The compound of any one of the preceding Embodiments, wherein a monocyclic unit is a phenyl ring.
802. The compound of any one of the preceding Embodiments, wherein a monocyclic unit is a 5-membered heteroaryl having 1-4 heteroatoms.
803. The compound of any one of the preceding Embodiments, wherein a monocyclic unit is a 6-membered heteroaryl having 1-4 heteroatoms.
804. The compound of any one of the preceding Embodiments, wherein a monocyclic ring is a saturated or partially unsaturated C₃₋₇ cycloaliphatic.
805. The compound of any one of the preceding Embodiments, wherein a monocyclic ring is a saturated or partially unsaturated 3-membered heterocyclyl having 1-4 heteroatoms.
806. A compound comprising one or more (e.g., 1, 2, 3, 4, 5, 6, 7, 8, 9, or 10) monomeric units, wherein each monomer is independently a compound of any one of the preceding Embodiments.
807. A compound having the structure of [A]-L^{D}_[B] or a salt thereof, wherein L^{D} is L as described in the preceding Embodiments, and each A and B independently has such a structure that each of A-H and B—H is independently a compound of any one of Embodiments 1-805.
808. The compound of Embodiment 807, wherein A-H and B—H has the same structure.
809. The compound of any one of the preceding Embodiments, wherein each heteroatom is independently selected from nitrogen, oxygen, sulfur, silicon, and phosphorus.
810. The compound of any one of the preceding Embodiments, wherein each heteroatom is independently selected from nitrogen, oxygen, and sulfur.
811. The compound of any one of the preceding Embodiments, wherein the compound activates FXR.
812. The compound of any one of the preceding Embodiments, wherein the compound activates FXR with an EC50 about or no more than about 10, 5, 2, 1, 0.5, 0.2, or 0.1 uM.
813. The compound of any one of the preceding Embodiments, wherein the compound activates FXR with an EC50 about or no more than about 5 uM.
814. The compound of any one of the preceding Embodiments, wherein the compound activates FXR with an EC50 about or no more than about 1 uM.
815. The compound of any one of the preceding Embodiments, wherein the compound activates TGR5.
816. The compound of any one of the preceding Embodiments, wherein the compound activates TGR5 with an EC50 about or no more than about 10, 5, 2, 1, 0.5, 0.2, or 0.1 uM.
817. The compound of any one of the preceding Embodiments, wherein the compound activates TGR5 with an EC50 about or no more than about 5 uM.
818. The compound of any one of the preceding Embodiments, wherein the compound activates TGR5 with an EC50 about or no more than about 1 uM.
819. The compound of any one of the preceding Embodiments, wherein the compound activates MRGPRX4 with an EC50 no less than about 10, 20 50, 100, 200, 500, or 1000 uM.
820. The compound of any one of the preceding Embodiments, wherein the compound activates MRGPRX4 with an EC50 no less than about 20 uM.
821. The compound of any one of the preceding Embodiments, wherein the compound activates MRGPRX4 with an EC50 no less than about 50 uM.
822. The compound of any one of the preceding Embodiments, wherein the compound activates MRGPRX4 with an EC50 no less than about 100 uM.
823. The compound of any one of the preceding Embodiments, wherein the EC50 of the compound for MRGPRX4 is about or at least about 2 fold of that of a reference compound.
824. The compound of any one of the preceding Embodiments, wherein the EC50 of the compound for MRGPRX4 is about or at least about 5 fold of that of a reference compound.
825. The compound of any one of the preceding Embodiments, wherein the EC50 of the compound for MRGPRX4 is about or at least about 10 fold of that of a reference compound.
826. The compound of any one of the preceding Embodiments, wherein the EC50 of the compound for MRGPRX4 is about or at least about 20 fold of that of a reference compound.
827. The compound of any one of the preceding Embodiments, wherein the EC50 of the compound for MRGPRX4 is about or at least about 50 fold of that of a reference compound.
828. The compound of any one of the preceding Embodiments, wherein the EC50 of the compound for MRGPRX4 is about or at least about 100 fold of that of a reference compound.
829. The compound of any one of the preceding Embodiments, wherein a reference compound is obeticholic acid or a salt thereof.
830. The compound of any one of the preceding Embodiments, wherein none of R¹ and R^{1a} in the compound is —OH, and a reference compound is an otherwise identical compound except that one of R′ and R^{1a} in the reference compound is —OH.
831. The compound of any one of the preceding Embodiments, wherein a reference compound is CDCA or a salt thereof.
832. The compound of any one of the preceding Embodiments, wherein a reference compound is cholic acid or a salt thereof.
833. The compound of any one of the preceding Embodiments, wherein MRGPRX4 activation is not observed at about 1 uM.
834. The compound of any one of the preceding Embodiments, wherein MRGPRX4 activation is not observed at about 2 uM.
835. The compound of any one of the preceding Embodiments, wherein MRGPRX4 activation is not observed at about 5 uM.
836. The compound of any one of the preceding Embodiments, wherein MRGPRX4 activation is not observed at about 10 uM.
837. The compound of any one of the preceding Embodiments, wherein MRGPRX4 activation is not observed at about 100 uM.
838. The compound of any one of the preceding Embodiments, wherein the EC50 for MRGPRX4 is about or at least about 10, 20, 50, 100, 200 or 500 fold compared to the EC50 for FXR.
839. The compound of any one of the preceding Embodiments, wherein the EC50 for MRGPRX4 is about or at least about 10 fold compared to the EC50 for FXR.
840. The compound of any one of the preceding Embodiments, wherein the EC50 for MRGPRX4 is about or at least about 50 fold compared to the EC50 for FXR.
841. The compound of any one of the preceding Embodiments, wherein the EC50 for MRGPRX4 is about or at least about 100 fold compared to the EC50 for FXR.
842. The compound of any one of the preceding Embodiments, wherein the EC50 for MRGPRX4 is about or at least about 10, 20, 50, 100, 200 or 500 fold compared to the EC50 for TGR5.
843. The compound of any one of the preceding Embodiments, wherein the EC50 for MRGPRX4 is about or at least about 10 fold compared to the EC50 for TGR5.
844. The compound of any one of the preceding Embodiments, wherein the EC50 for MRGPRX4 is about or at least about 50 fold compared to the EC50 for TGR5.
845. The compound of any one of the preceding Embodiments, wherein the EC50 for MRGPRX4 is about or at least about 100 fold compared to the EC50 for TGR5.
846. The compound of any one of the preceding Embodiments, wherein the ratio of the EC50 for MRGPRX4 over the EC50 for FXR is about or at least about 2, 5, 10, 20, 50 or 100 fold of the ratio of a reference compound.
847. The compound of any one of the preceding Embodiments, wherein the ratio of the EC50 for MRGPRX4 over the EC50 for FXR is about or at least about 2 fold of the ratio of a reference compound.
848. The compound of any one of the preceding Embodiments, wherein the ratio of the EC50 for MRGPRX4 over the EC50 for FXR is about or at least about 5 fold of the ratio of a reference compound.
849. The compound of any one of the preceding Embodiments, wherein the ratio of the EC50 for MRGPRX4 over the EC50 for FXR is about or at least about 10 fold of the ratio of a reference compound.
850. The compound of any one of the preceding Embodiments, wherein the ratio of the EC50 for MRGPRX4 over the EC50 for FXR is about or at least about 20 fold of the ratio of a reference compound.
851. The compound of any one of the preceding Embodiments, wherein the ratio of the EC50 for MRGPRX4 over the EC50 for FXR is about or at least about 50 fold of the ratio of a reference compound.
852. The compound of any one of the preceding Embodiments, wherein the ratio of the EC50 for MRGPRX4 over the EC50 for FXR is about or at least about 100 fold of the ratio of a reference compound.
853. The compound of any one of the preceding Embodiments, wherein the ratio of the EC50 for MRGPRX4 over the EC50 for TGR5 is about or at least about 2, 5, 10, 20, 50 or 100 fold of the ratio of a reference compound.
854. The compound of any one of the preceding Embodiments, wherein the ratio of the EC50 for MRGPRX4 over the EC50 for TGR5 is about or at least about 2 fold of the ratio of a reference compound.
855. The compound of any one of the preceding Embodiments, wherein the ratio of the EC50 for MRGPRX4 over the EC50 for TGR5 is about or at least about 5 fold of the ratio of a reference compound.
856. The compound of any one of the preceding Embodiments, wherein the ratio of the EC50 for MRGPRX4 over the EC50 for TGR5 is about or at least about 10 fold of the ratio of a reference compound.
857. The compound of any one of the preceding Embodiments, wherein the ratio of the EC50 for MRGPRX4 over the EC50 for TGR5 is about or at least about 20 fold of the ratio of a reference compound.
858. The compound of any one of the preceding Embodiments, wherein the ratio of the EC50 for MRGPRX4 over the EC50 for TGR5 is about or at least about 50 fold of the ratio of a reference compound.
859. The compound of any one of the preceding Embodiments, wherein the ratio of the EC50 for MRGPRX4 over the EC50 for TGR5 is about or at least about 100 fold of the ratio of a reference compound.
860. The compound of any one of the preceding Embodiments, wherein efficacy of the compound is about or at least about 80% of a reference compound.
861. The compound of any one of the preceding Embodiments, wherein efficacy of the compound is about or at least about 90% of a reference compound.
862. The compound of any one of the preceding Embodiments, wherein efficacy of the compound is about or at least about 100% of a reference compound.
863. The compound of any one of the preceding Embodiments, wherein efficacy of the compound is about or at least about 150% of a reference compound.
864. The compound of any one of the preceding Embodiments, wherein efficacy of the compound is about or at least about 200% of a reference compound.
865. The compound of any one of the preceding Embodiments, wherein efficacy of the compound is about or at least about 250% of a reference compound.
866. The compound of any one of the preceding Embodiments, wherein efficacy of the compound is about or at least about 300% of a reference compound.
867. The compound of any one of the preceding Embodiments, wherein efficacy of the compound is about or at least about 400% of a reference compound.
868. The compound of any one of Embodiments 846-867, wherein a reference compound is obeticholic acid or a salt thereof.
869. The compound of any one of Embodiments 846-867, wherein none of R¹ and R^{1a} in the compound is —OH, and a reference compound is an otherwise identical compound except that one of R¹ and R^{1a} in the reference compound is —OH.
870. The compound of any one of Embodiments 846-867, wherein a reference compound is CDCA or a salt thereof.
871. The compound of any one of Embodiments 846-867, wherein a reference compound is cholic acid or a salt thereof.
872. The compound of any one of Embodiments 860-871, wherein the efficacy is for FXR activation.
873. The compound of any one of Embodiments 860-872, wherein the efficacy is for TGR5 activation.
874. The compound of any one of the preceding Embodiments, wherein the compound activates FXR at a level that is about or at least about 50% of that of a reference compound when assesses at the same concentration.
875. The compound of Embodiment 874, wherein the level of the compound is about or at least about 75% of that of a reference compound.
876. The compound of Embodiment 874, wherein the level of the compound is about or at least about 80% of that of a reference compound.
877. The compound of Embodiment 874, wherein the level of the compound is about or at least about 90% of that of a reference compound.
878. The compound of Embodiment 874, wherein the level of the compound is about or at least about 100% of that of a reference compound.
879. The compound of Embodiment 874, wherein the level of the compound is about or at least about 150% of that of a reference compound.
880. The compound of Embodiment 874, wherein the level of the compound is about or at least about 200% of that of a reference compound.
881. The compound of Embodiment 874, wherein the level of the compound is about or at least about 250% of that of a reference compound.
882. The compound of Embodiment 874, wherein the level of the compound is about or at least about 300% of that of a reference compound.
883. The compound of Embodiment 874, wherein the level of the compound is about or at least about 400% of that of a reference compound.
884. The compound of Embodiment 874, wherein the level of the compound is about or at least about 500% of that of a reference compound.
885. The compound of any one of Embodiments 874-884, wherein the concentration is about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM.
886. The compound of any one of Embodiments 874-908, wherein the concentration is about 10², 10^{1.5}, 10¹, 10^{0.5}, 10⁰, 10^{0.5}, 10⁻¹, 10^{-1.5}, 10⁻², 10^{-2.5}, 10⁻³, 10^{-3.5}, or 10⁻⁴ uM.
887. The compound of any one of Embodiments 874-886, wherein the concentration is about 100 uM.
888. The compound of any one of Embodiments 874-886, wherein the concentration is about 80 uM.
889. The compound of any one of Embodiments 874-886, wherein the concentration is about 50 uM.
890. The compound of any one of Embodiments 874-886, wherein the concentration is about 20 uM.
891. The compound of any one of Embodiments 874-886, wherein the concentration is about 10 uM.
892. The compound of any one of Embodiments 874-886, wherein the concentration is about 5 uM.
893. The compound of any one of Embodiments 874-886, wherein the concentration is about 1 uM.
894. The compound of any one of Embodiments 874-886, wherein the concentration is about 0.5 uM.
895. The compound of any one of Embodiments 874-886, wherein the concentration is about 0.2 uM.
896. The compound of any one of Embodiments 874-886, wherein the concentration is about 0.1 uM.
897. The compound of any one of Embodiments 874-884, wherein the concentration is about or at least about EC50 of the compound for FXR.
898. The compound of any one of Embodiments 874-884, wherein the concentration is about or at least about EC80 of the compound for FXR.
899. The compound of any one of Embodiments 874-884, wherein the concentration is about or at least about EC90 of the compound for FXR.
900. The compound of any one of Embodiments 874-884, wherein the concentration is about or at least about EC100 of the compound for FXR.
901. The compound of any one of Embodiments 874-884, wherein the concentration is about or at least about EC50 of the reference compound for FXR.
902. The compound of any one of Embodiments 874-884, wherein the concentration is about or at least about EC80 of the reference compound for FXR.
903. The compound of any one of Embodiments 874-884, wherein the concentration is about or at least about EC90 of the reference compound for FXR.
904. The compound of any one of Embodiments 874-884, wherein the concentration is about or at least about EC100 of the reference compound for FXR.
905. The compound of any one of Embodiments 874-904, wherein a reference compound is obeticholic acid or a salt thereof.
906. The compound of any one of Embodiments 874-904, wherein none of R¹ and R^{1a} in the compound is —OH, and a reference compound is an otherwise identical compound except that one of R¹ and R^{1a} in the reference compound is —OH.
907. The compound of any one of Embodiments 874-904, wherein a reference compound is CDCA or a salt thereof.
908. The compound of any one of Embodiments 874-904, wherein a reference compound is cholic acid or a salt thereof.
909. The compound of any one of the preceding Embodiments, wherein the compound activates TGR5 at a level that is about or at least about 50% of that of a reference compound when assesses at the same concentration.
910. The compound of Embodiment 909, wherein the level of the compound is about or at least about 75% of that of a reference compound.
911. The compound of Embodiment 909, wherein the level of the compound is about or at least about 80% of that of a reference compound.
912. The compound of Embodiment 909, wherein the level of the compound is about or at least about 90% of that of a reference compound.
913. The compound of Embodiment 909, wherein the level of the compound is about or at least about 100% of that of a reference compound.
914. The compound of Embodiment 909, wherein the level of the compound is about or at least about 150% of that of a reference compound.
915. The compound of Embodiment 909, wherein the level of the compound is about or at least about 200% of that of a reference compound.
916. The compound of Embodiment 909, wherein the level of the compound is about or at least about 250% of that of a reference compound.
917. The compound of Embodiment 909, wherein the level of the compound is about or at least about 300% of that of a reference compound.
918. The compound of Embodiment 909, wherein the level of the compound is about or at least about 400% of that of a reference compound.
919. The compound of Embodiment 909, wherein the level of the compound is about or at least about 500% of that of a reference compound.
920. The compound of any one of Embodiments 909-919, wherein the concentration is about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM.
921. The compound of any one of Embodiments 909-919, wherein the concentration is about 10², 10^{1.5}, 10¹, 10^{0.5}, 10⁰, 10^{-0.5}, 10⁻¹, 10^{-1.5}, 10⁻², 10^{-2.5}, 10⁻³, 10^{-3.5}, or 10⁻⁴ uM.
922. The compound of any one of Embodiments 909-921, wherein the concentration is about 100 uM.
923. The compound of any one of Embodiments 909-921, wherein the concentration is about 80 uM.
924. The compound of any one of Embodiments 909-921, wherein the concentration is about 50 uM.
925. The compound of any one of Embodiments 909-921, wherein the concentration is about 20 uM.
926. The compound of any one of Embodiments 909-921, wherein the concentration is about 10 uM.
927. The compound of any one of Embodiments 909-921, wherein the concentration is about 5 uM.
928. The compound of any one of Embodiments 909-921, wherein the concentration is about 1 uM.
929. The compound of any one of Embodiments 909-921, wherein the concentration is about 0.5 uM.
930. The compound of any one of Embodiments 909-921, wherein the concentration is about 0.2 uM.
931. The compound of any one of Embodiments 909-921, wherein the concentration is about 0.1 uM.
932. The compound of any one of Embodiments 909-919, wherein the concentration is about or at least about EC50 of the compound for TGR5.
933. The compound of any one of Embodiments 909-919, wherein the concentration is about or at least about EC80 of the compound for TGR5.
934. The compound of any one of Embodiments 909-919, wherein the concentration is about or at least about EC90 of the compound for TGR5.
935. The compound of any one of Embodiments 909-919, wherein the concentration is about or at least about EC100 of the compound for TGR5.
936. The compound of any one of Embodiments 909-919, wherein the concentration is about or at least about EC50 of the reference compound for TGR5.
937. The compound of any one of Embodiments 909-919, wherein the concentration is about or at least about EC80 of the reference compound for TGR5.
938. The compound of any one of Embodiments 909-919, wherein the concentration is about or at least about EC90 of the reference compound for TGR5.
939. The compound of any one of Embodiments 909-919, wherein the concentration is about or at least about EC100 of the reference compound for TGR5.
940. The compound of any one of Embodiments 909-939, wherein a reference compound is obeticholic acid or a salt thereof.
941. The compound of any one of Embodiments 909-939, wherein none of R¹ and R^{1a} in the compound is —OH, and a reference compound is an otherwise identical compound except that one of R¹ and R^{1a} in the reference compound is —OH.
942. The compound of any one of Embodiments 909-939, wherein a reference compound is CDCA or a salt thereof.
943. The compound of any one of Embodiments 909-939, wherein a reference compound is cholic acid or a salt thereof.
944. The compound of any one of the preceding Embodiments, wherein the compound activates MRGPRX4 at a level that is about or no more than about 50% of that of a reference compound when assesses at the same concentration.
945. The compound of Embodiment 944, wherein the level of the compound is about or no more than about 40% of that of a reference compound.
946. The compound of Embodiment 944, wherein the level of the compound is about or no more than about 30% of that of a reference compound.
947. The compound of Embodiment 944, wherein the level of the compound is about or no more than about 20% of that of a reference compound.
948. The compound of Embodiment 944, wherein the level of the compound is about or no more than about 10% of that of a reference compound.
949. The compound of Embodiment 944, wherein the level of the compound is about or no more than about 5% of that of a reference compound.
950. The compound of Embodiment 944, wherein the compound does not activate MRGPRX4 while the reference compound activates MRGPRX4.
951. The compound of any one of Embodiments 944-950, wherein the concentration is about or at least about EC10 of the reference compound for MRGPRX4.
952. The compound of any one of Embodiments 944-950, wherein the concentration is about or at least about EC20 of the reference compound for MRGPRX4.
953. The compound of any one of Embodiments 944-950, wherein the concentration is about or at least about EC30 of the reference compound for MRGPRX4.
954. The compound of any one of Embodiments 944-950, wherein the concentration is about or at least about EC40 of the reference compound for MRGPRX4.
955. The compound of any one of Embodiments 944-950, wherein the concentration is about or at least about EC50 of the reference compound for MRGPRX4.
956. The compound of any one of Embodiments 944-950, wherein the concentration is about or at least about EC70 of the reference compound for MRGPRX4.
957. The compound of any one of Embodiments 944-950, wherein the concentration is about or at least about EC80 of the reference compound for MRGPRX4.
958. The compound of any one of Embodiments 944-950, wherein the concentration is about or at least about EC90 of the reference compound for MRGPRX4.
959. The compound of any one of Embodiments 944-950, wherein the concentration is about or at least about EC100 of the reference compound for MRGPRX4.
960. The compound of any one of Embodiments 944-959, wherein the concentration is about or at least about EC10 of the compound for MRGPRX4 (if the compound is observed to activate MRGPRX4).
961. The compound of any one of Embodiments 944-959, wherein the concentration is about or at least about EC20 of the compound for MRGPRX4 (if the compound is observed to activate MRGPRX4).
962. The compound of any one of Embodiments 944-959, wherein the concentration is about or at least about EC30 of the compound for MRGPRX4 (if the compound is observed to activate MRGPRX4).
963. The compound of any one of Embodiments 944-959, wherein the concentration is about or at least about EC40 of the compound for MRGPRX4 (if the compound is observed to activate MRGPRX4).
964. The compound of any one of Embodiments 944-959, wherein the concentration is about or at least about EC50 of the compound for MRGPRX4 (if the compound is observed to activate MRGPRX4).
965. The compound of any one of Embodiments 944-959, wherein the concentration is about or at least about EC70 of the compound for MRGPRX4 (if the compound is observed to activate MRGPRX4).
966. The compound of any one of Embodiments 944-959, wherein the concentration is about or at least about EC80 of the compound for MRGPRX4 (if the compound is observed to activate MRGPRX4).
967. The compound of any one of Embodiments 944-959, wherein the concentration is about or at least about EC90 of the compound for MRGPRX4 (if the compound is observed to activate MRGPRX4).
968. The compound of any one of Embodiments 944-959, wherein the concentration is about or at least about EC100 of the compound for MRGPRX4 (if the compound is observed to activate MRGPRX4).
969. The compound of any one of Embodiments 944-968, wherein the concentration is about or at least about 2 fold of the EC50 of the compound for FXR.
970. The compound of Embodiment 969, wherein the concentration is about or at least about 5 fold of the EC50 of the compound for FXR.
971. The compound of Embodiment 969, wherein the concentration is about or at least about 10 fold of the EC50 of the compound for FXR.
972. The compound of Embodiment 969, wherein the concentration is about or at least about 20 fold of the EC50 of the compound for FXR.
973. The compound of Embodiment 969, wherein the concentration is about or at least about 50 fold of the EC50 of the compound for FXR.
974. The compound of Embodiment 969, wherein the concentration is about or at least about 100 fold of the EC50 of the compound for FXR.
975. The compound of Embodiment 969, wherein the concentration is about or at least about 200 fold of the EC50 of the compound for FXR.
976. The compound of Embodiment 969, wherein the concentration is about or at least about 500 fold of the EC50 of the compound for FXR.
977. The compound of Embodiment 969, wherein the concentration is about or at least about 1000 fold of the EC50 of the compound for FXR.
978. The compound of Embodiment 969, wherein the concentration is about or at least about 5 fold of the EC50 of the compound for FXR.
979. The compound of any one of Embodiments 944-978, wherein the concentration is about or at least about 2 fold of the EC50 of the compound for TGR5.
980. The compound of Embodiment 979, wherein the concentration is about or at least about 5 fold of the EC50 of the compound for TGR5.
981. The compound of Embodiment 979, wherein the concentration is about or at least about 10 fold of the EC50 of the compound for TGR5.
982. The compound of Embodiment 979, wherein the concentration is about or at least about 20 fold of the EC50 of the compound for TGR5.
983. The compound of Embodiment 979, wherein the concentration is about or at least about 50 fold of the EC50 of the compound for TGR5.
984. The compound of Embodiment 979, wherein the concentration is about or at least about 100 fold of the EC50 of the compound for TGR5.
985. The compound of Embodiment 979, wherein the concentration is about or at least about 200 fold of the EC50 of the compound for TGR5.
986. The compound of Embodiment 979, wherein the concentration is about or at least about 500 fold of the EC50 of the compound for TGR5.
987. The compound of Embodiment 979, wherein the concentration is about or at least about 1000 fold of the EC50 of the compound for TGR5.
988. The compound of Embodiment 979, wherein the concentration is about or at least about 5 fold of the EC50 of the compound for TGR5.
989. The compound of any one of Embodiments 944-950, wherein the concentration is about 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.05, or 0.01 uM.
990. The compound of any one of Embodiments 944-950, wherein the concentration is about 10², 10^{1.5}, 10¹, 10^{0.5}, 10⁰, 10^{-0.5}, 10⁻¹, 10^{-1.5}, 10⁻², 10^{-2.5}, 10⁻³, 10^{-3.5}, or 10⁻⁴ uM.
991. The compound of any one of Embodiments 944-950, wherein the concentration is about 100 uM.
992. The compound of any one of Embodiments 944-950, wherein the concentration is about 80 uM.
993. The compound of any one of Embodiments 944-950, wherein the concentration is about 50 uM.
994. The compound of any one of Embodiments 944-950, wherein the concentration is about 20 uM.
995. The compound of any one of Embodiments 944-950, wherein the concentration is about 10 uM.
996. The compound of any one of Embodiments 944-950, wherein the concentration is about 5 uM.
997. The compound of any one of Embodiments 944-950, wherein the concentration is about 1 uM.
998. The compound of any one of Embodiments 944-950, wherein the concentration is about 0.5 uM.
999. The compound of any one of Embodiments 944-950, wherein the concentration is about 0.2 uM.
1000. The compound of any one of Embodiments 944-950, wherein the concentration is about 0.1 uM.
1001. The compound of any one of Embodiments 944-950, wherein the concentration is an effective concentration for preventing or treating a condition, disorder or disease.
1002. The compound of any one of Embodiments 944-950, wherein the concentration is provided by administration of an effective amount utilized in a method described herein.
1003. The compound of any one of Embodiments 944-950, wherein the concentration is provided by administration of an effective amount utilized in a method of any one of the Embodiments.
1004. The compound of any one of Embodiments 944-1003, wherein a reference compound is obeticholic acid or a salt thereof.
1005. The compound of any one of Embodiments 944-1003, wherein none of R¹ and R^{1a} in the compound is —OH, and a reference compound is an otherwise identical compound except that one of R¹ and R^{1a} in the reference compound is —OH.
1006. The compound of any one of Embodiments 944-1003, wherein a reference compound is CDCA or a salt thereof.
1007. The compound of any one of Embodiments 944-1003, wherein a reference compound is cholic acid or a salt thereof.
1008. A compound, wherein the compound is compound 21 or a salt thereof.
1009. A compound, wherein the compound is compound 53 or a salt thereof.
1010. A compound, wherein the compound is compound 55 or a salt thereof.
1011. A compound, wherein the compound is compound 77 or a salt thereof.
1012. A compound, wherein the compound is compound 73 or a salt thereof.
1013. A compound, wherein the compound is compound 1-4 or a salt thereof.
1014. A compound, wherein the compound is compound 1-5 or a salt thereof.
1015. A compound, wherein the compound is compound 2-1 or a salt thereof.
1016. A compound, wherein the compound is compound 2-6 or a salt thereof.
1017. A compound, wherein the compound is compound 3-1 or a salt thereof.
1018. A compound, wherein the compound is compound 3-3 or a salt thereof.
1019. A compound, wherein the compound is compound 4-1 or a salt thereof.
1020. A compound, wherein the compound is compound 4-2 or a salt thereof.
1021. A compound, wherein the compound is compound 4-3 or a salt thereof.
1022. A compound, wherein the compound is compound 4-4 or a salt thereof.
1023. A compound, wherein the compound is compound 5-1 or a salt thereof.
1024. A compound, wherein the compound is compound 5-2 or a salt thereof.
1025. A compound, wherein the compound is compound 5-3 or a salt thereof.
1026. A compound, wherein the compound is compound 5-4 or a salt thereof.
1027. A compound, wherein the compound is compound 5-5 or a salt thereof.
1028. A compound, wherein the compound is compound 5-7 or a salt thereof.
1029. A compound, wherein the compound is compound 5-8 or a salt thereof.
1030. A compound, wherein the compound is compound 5-9 or a salt thereof.
1031. A compound, wherein the compound is compound 5-10 or a salt thereof.
1032. A compound, wherein the compound is compound 5-11 or a salt thereof.
1033. A compound, wherein the compound is compound 5-13 or a salt thereof.
1034. A compound, wherein the compound is compound 5-14 or a salt thereof.
1035. A compound, wherein the compound is compound 5-15 or a salt thereof.
1036. A compound, wherein the compound is compound 5-16 or a salt thereof.
1037. A compound, wherein the compound is compound 5-17 or a salt thereof.
1038. A compound, wherein the compound is compound 5-18 or a salt thereof.
1039. A compound, wherein the compound is compound 5-19 or a salt thereof.
1040. A compound, wherein the compound is compound 5-20 or a salt thereof.
1041. A compound, wherein the compound is compound 5-21 or a salt thereof.
1042. A compound, wherein the compound is compound 5-22 or a salt thereof.
1043. A compound, wherein the compound is compound 5-23 or a salt thereof.
1044. A compound, wherein the compound is compound 5-24 or a salt thereof.
1045. A compound, wherein the compound is compound 5-25 or a salt thereof.
1046. A compound, wherein the compound is compound 5-26 or a salt thereof.
1047. A compound, wherein the compound is compound 5-27 or a salt thereof.
1048. A compound, wherein the compound is compound 5-29 or a salt thereof.
1049. A compound, wherein the compound is compound 5-30 or a salt thereof.
1050. A compound, wherein the compound is compound 6-1 or a salt thereof.
1051. A compound, wherein the compound is compound 6-2 or a salt thereof.
1052. A compound, wherein the compound is compound 7-1 or a salt thereof.
1053. A compound, wherein the compound is compound 7-2 or a salt thereof.
1054. A compound, wherein the compound is compound 7-3 or a salt thereof.
1055. A compound, wherein the compound is compound 8-2 or a salt thereof.
1056. A compound, wherein the compound is compound 8-3 or a salt thereof.
1057. A compound, wherein the compound is compound 8-4 or a salt thereof.
1058. A compound, wherein the compound is compound 8-5 or a salt thereof.
1059. A compound, wherein the compound is compound 9-1 or a salt thereof.
1060. A compound, wherein the compound is compound 9-2 or a salt thereof.
1061. A compound, wherein the compound is compound 9-3 or a salt thereof.
1062. A compound, wherein the compound is compound 9-4 or a salt thereof.
1063. A compound, wherein the compound is compound 9-5 or a salt thereof.
1064. A compound, wherein the compound is compound 9-6 or a salt thereof.
1065. A compound, wherein the compound is compound 10-1 or a salt thereof.
1066. A compound, wherein the compound is compound 10-2 or a salt thereof.
1067. A compound, wherein the compound is compound 10-3 or a salt thereof.
1068. A compound, wherein the compound is compound 10-4 or a salt thereof.
1069. A compound, wherein the compound is compound II-1 or a salt thereof.
1070. A compound, wherein the compound is compound II-3 or a salt thereof.
1071. A compound, wherein the compound is compound II-4 or a salt thereof.
1072. A compound, wherein the compound is compound 12-1 or a salt thereof.
1073. A compound, wherein the compound is compound 12-2 or a salt thereof.
1074. A compound, wherein the compound is compound 13-2 or a salt thereof.
1075. A compound, wherein the compound is compound 14-1 or a salt thereof.
1076. A compound, wherein the compound is compound 14-3 or a salt thereof.
1077. A compound, wherein the compound is compound 14-4 or a salt thereof.
1078. A compound, wherein the compound is compound 15-2 or a salt thereof.
1079. A compound, wherein the compound is compound 16-2 or a salt thereof.
1080. A compound, wherein the compound is compound 17-1 or a salt thereof.
1081. A compound, wherein the compound is compound 17-2 or a salt thereof.
1082. A compound, wherein the compound is compound 18-1 or a salt thereof.
1083. A compound, wherein the compound is compound 18-2 or a salt thereof.
1084. A compound, wherein the compound is compound 18-3 or a salt thereof.
1085. A compound, wherein the compound is compound 19-1 or a salt thereof.
1086. A compound, wherein the compound is compound 19-2 or a salt thereof.
1087. A compound, wherein the compound is compound 19-3 or a salt thereof.
1088. A compound, wherein the compound is compound 20-1 or a salt thereof.
1089. A compound, wherein the compound is compound 20-2 or a salt thereof.
1090. A compound, wherein the compound is compound 20-3 or a salt thereof.
1091. A compound, wherein the compound is compound 21-1 or a salt thereof.
1092. A compound, wherein the compound is compound 22-1 or a salt thereof.
1093. A compound, wherein the compound is compound 23-1 or a salt thereof.
1094. A compound, wherein the compound is compound 24-1 or a salt thereof.
1095. A pharmaceutical composition, comprising or delivering a compound of any one of the preceding Embodiments or a pharmaceutically acceptable salt thereof and pharmaceutically acceptable carrier.
1096. A composition comprising a compound of any one of the preceding Embodiments or a salt thereof, wherein one or more isotopes are enriched at one or more locations.
1097. The composition of Embodiment 1096, wherein the composition is enriched for D at R¹.
1098. The composition of any one of Embodiments 1096-1097, wherein the composition is enriched for D at R^{1a}.
1099. The composition of any one of Embodiments 1096-1098, wherein at each location the enrichment is independently about or at least about 10, 50, 100, 500, 1000 fold or the natural abundance of the isotope.
1100. The composition of any one of Embodiments 1096-1098, wherein at each location the enrichment is independently about or at least about 100 fold or the natural abundance of the isotope.
1101. The composition of any one of Embodiments 1096-1098, wherein at each location the enrichment is independently about or at least about 1000 fold or the natural abundance of the isotope.
1102. The composition of any one of Embodiments 1096-1098, wherein at each location independently about or at least about 1%, 2%, 5%, 10%, 20%, 50%, 60%, 70%, 80%, 90%, 95% or 97% of all molecules of the compound or a salt thereof have the isotope at the location.
1103. The composition of Embodiment 1102, wherein the percentage is about or at least about 1%.
1104. The composition of Embodiment 1102, wherein the percentage is about or at least about 10%.
1105. The composition of Embodiment 1102, wherein the percentage is about or at least about 50%.
1106. The composition of Embodiment 1102, wherein the percentage is about or at least about 90%.
1107. The composition of any one of Embodiments 1096-1106, wherein the composition is a pharmaceutical composition of Embodiment 1095.
1108. A method comprising
   assessing activation of FXR and/or TGR5 by a compound and a reference compound;
   assessing activation of MRGPRX4 by the compound and the reference compound;
   wherein the compound provides higher selectivity for the activation of FXR and/or TGR5 over MRGPRX4.
1109. The method of Embodiment 1108, wherein the compound comprises moiety A.
1110. The method of Embodiment 1108, wherein the compound is a bile acid or a bile acid derivative, or a salt thereof.
1111. The method of Embodiment 1108, wherein the compound is a compound of any one of the preceding Embodiments or a salt thereof.
1112. A method for activating FXR, comprising contacting FXR with a compound of any one of the preceding Embodiments or a salt thereof, or a composition of any one of the preceding Embodiments.
1113. A method for activating FXR in a system (e.g., a cell, a tissue, an organ, a subject, etc.), comprising administering or delivering to the system a compound of any one of the preceding Embodiments or a salt thereof, or a composition of any one of the preceding Embodiments.
1114. A method for activating TGR5, comprising contacting TGR5 with a compound of any one of the preceding Embodiments or a salt thereof, or a composition of any one of the preceding Embodiments.
1115. A method for activating TGR5 in a system (e.g., a cell, a tissue, an organ, a subject, etc.), comprising administering or delivering to the system a compound of any one of the preceding Embodiments or a salt thereof, or a composition of any one of the preceding Embodiments.
1116. A method for preventing a condition, disorder or disease in a subject, comprising administering or delivering to a subject susceptible thereto an effective amount of a compound or a salt thereof or composition of any one of the preceding Embodiments.
1117. A method for treating a condition, disorder or disease in a subject, comprising administering or delivering to a subject suffering therefrom an effective amount of a compound or a salt thereof or a composition of any one of the preceding Embodiments.
1118. The method of any one of Embodiments 1116-1117, wherein the subject benefits from FXR activation.
1119. The method of any one of Embodiments 1116-1117, wherein the subject benefits from TGR5 activation.
1120. The method of any one of Embodiments 1116-1117, wherein a condition, disorder or disease is a hepatic condition, disorder or disease.
1121. The method of any one of Embodiments 1116-1117, wherein a condition, disorder or disease is a bile acid synthesis condition, disorder or disease.
1122. The method of any one of Embodiments 1116-1117, wherein a condition, disorder or disease is NASH.
1123. The method of any one of Embodiments 1116-1117, wherein a condition, disorder or disease is primary biliary cholangitis.
1124. The method of any one of Embodiments 1116-1117, wherein a condition, disorder or disease is primary biliary cholangitis with cirrhosis.
1125. The method of any one of Embodiments 1116-1117, wherein a condition, disorder or disease is primary biliary cholangitis with compensated cirrhosis.
1126. The method of any one of Embodiments 1116-1117, wherein a condition, disorder or disease is peroxisomal disorders (PDs).
1127. The method of any one of Embodiments 1116-1117, wherein a condition, disorder or disease is peroxisomal disorders (PDs) including Zellweger spectrum disorders in a subject who exhibits manifestations of liver disease, steatorrhea or complications from decreased fat soluble vitamin absorption.
1128. The method of any one of Embodiments 1116-1117, wherein a condition, disorder or disease is neurodegenerative condition, disorder or disease.
1129. The method of any one of Embodiments 1116-1117, wherein a condition, disorder or disease is amyotrophic lateral sclerosis.
1130. The method of any one of the preceding Embodiments, comprising administering or delivering another therapeutic agent.
1131. The method of Embodiment 1130, wherein another therapeutic agent is phenylbutyric acid or a salt thereof.
1132. The method of Embodiment 1130, wherein another therapeutic agent is phenylbutyric acid or a pharmaceutically acceptable salt.
1133. The method of Embodiment 1130, wherein another therapeutic agent is sodium phenylbutyrate.
1134. The method of any one of the preceding Embodiments, comprising administering or delivering another therapeutic agent that is or comprises a GPCR inhibitor, wherein activation of the GPCR is associated with itch.
1135. The method of any one of the preceding Embodiments, comprising administering or delivering another therapeutic agent that is or comprises a MRGPRX inhibitor.
1136. The method of any one of the preceding Embodiments, comprising administering or delivering another therapeutic agent that is or comprises a MRGPRX4 inhibitor.
1137. The method of any one of the preceding Embodiments, comprising administering or delivering another therapeutic agent that is or comprises a MRGPRB inhibitor.
1138. The method of any one of the preceding Embodiments, wherein an inhibitor is administered or delivered at a lower level compared to when a reference compound is administered or delivered.
1139. The method of any one of the preceding Embodiments, wherein the compound or a salt thereof or the composition is administer or delivered concurrently with another therapeutic agent.
1140. The method of any one of the preceding Embodiments, wherein the compound or a salt thereof or the composition is administer or delivered in the same composition as another therapeutic agent.
1141. The method of any one of the preceding Embodiments, wherein the compound or a salt thereof or the composition is administer or delivered prior to another therapeutic agent.
1142. The method of any one of the preceding Embodiments, wherein the compound or a salt thereof or the composition is administer or delivered after another therapeutic agent.
1143. The method of any one of the preceding Embodiments, wherein a subject is simultaneously exposed to a compound of any one of the preceding Embodiments and another therapeutic agent.
1144. The method of any one of the preceding Embodiments, wherein a side effect associated with an administered or delivered compound is less severe or removed compared to a reference compound.
1145. The method of Embodiment 1144, wherein a side effect is itch.
1146. The method of any one of the preceding Embodiments, wherein MRGPRX4 is not activated, or activated at a lower level comparing to a reference compound under comparable conditions.
1147. The method of any one of the preceding Embodiments, wherein none of R¹ and R^{1a} in the compound is —OH, and a reference compound is an otherwise identical compound except that one of R¹ and R^{1a} in the reference compound is —OH.
1148. The method of any one of the preceding Embodiments, wherein a reference compound is obeticholic acid or a salt thereof.
1149. The method of any one of the preceding Embodiments, wherein a reference compound is cholic acid or a salt thereof.
1150. A method for preparing a compound of any one of the preceding Embodiments using a method described in the specification.
1151. A method comprising:
   contacting a compound comprising a leaving group (e.g., —OS(O)₂R^{s} such as —OMs) and moiety A (e.g., a compound comprising 3-leaving group and 3-H bonded to moiety A, a compound of formula I wherein one of R¹ and R^{1a} is —H and the other is a leaving group, etc.) or a salt thereof with a reducing agent; and
   producing a compound moiety A but not the leaving group (e.g., a compound comprising two 3-H bonded to moiety A, a compound of formula I wherein both R¹ and R^{1a} are —H, etc.) or a salt thereof.
1152. The method of Embodiment 1151, wherein a compound comprising a leaving group is a compound of any one of the preceding Embodiments, wherein R¹ or R^{1a} is a leaving group.
1153. The method of Embodiment 1153, wherein a leaving group is -OMs.
1154. The method of any one of Embodiments 1151-1153, wherein a reducing agent is LiAlD₄.
1155. A method, comprising:
   contacting a compound having the structure of HO—C(O)-L″-Cy-S(O)₂N(R′)₂ or a salt thereof with a reducing agent to provide a compound having the structure of HO—CH₂-L″-Cy-S(O)₂N(R′)₂ or a salt thereof.
1156. The method of Embodiment 1155, wherein -Cy- is optionally substituted phenylene.
1157. The method of any one of Embodiments 1155-1156, wherein a reducing agent is NaBH₄.
1158. The method of any one of Embodiments 1155-1157, wherein the starting compound is

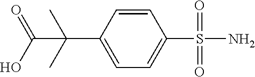 [SMILES: CC(C)(C(=O)O)c1ccc(S(N)(=O)=O)cc1]

or a salt thereof.
1159. The method of any one of Embodiments 1155-1157, wherein the starting compound is

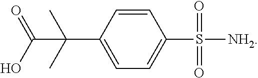 [SMILES: CC(C)(C(=O)O)c1ccc(S(N)(=O)=O)cc1]

1160. The method of any one of Embodiments 1155-1159, wherein the product compound is

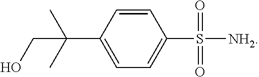

1. (canceled)

2. A compound, wherein the compound is a compound of formula I or a salt thereof: 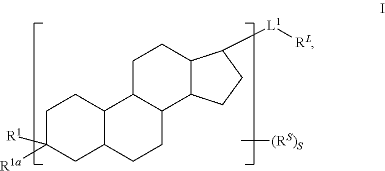
wherein:
each of R¹ and R^{1a} is independently —H or halogen;
each R^{s} is independently —H, -L″-R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, —N(R′)₂, a protected hydroxyl group, or R^{s} is 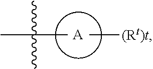
or two R^{s} attached to the same atom are taken together to form ═O or ═NR^{x};
t is 0-6;
each R^{t} is independently R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, or —N(R′)₂;
each Ring A is independently an optionally substituted 3-20 membered monocyclic, bicyclic or polycyclic ring having 0-10 heteroatoms;
each L″ is independently a covalent bond, or an optionally substituted, bivalent C₁₋₆ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
R^{L} is R^{s}, —C(O)R^{s}, —C(O)OR^{s}, —C(O)N(R^{s})₂, —C(O)N(R′)S(O)₂R^{s}, —C(O)N(R′)C(R′)₂C(O)N(R^{s})₂, —C(O)N(R′)C(R′)₂S(O)₂R^{s}, —C(O)N(R′)C(R′)₂S(O)₂N(R^{s})₂, —C(O)N(R′)C(R′)₂P(O)(R^{s})₂, —C(O)N(R′)C(R′)₂N(R′)C(O)N(R′)S(O)₂R^{s}, —C(O)N(R′)C(R)₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂₀R^{s}, —N(R^{s})₂, —N(R′)C(O)R^{s}, —N(R′)C(S)R^{s}, —N(R′)C(NR′)R^{s}, —N(R′)C(O)OR^{s}, —N(R′)C(O)N(R^{s})₂, —N(R′)C(NR′)N(R^{s})₂, —N(R′)C(O)N(R′)S(O)₂R′, —N(R′)C(S)N(R′)S(O)₂R^{s}, —N(R′)C(NR′)N(R′)S(O)₂R^{s}, —N(R′)C(O)C(O)N(R′)S(O)₂R′, —N(R′)C(O)N(R′)S(O)₂N(R^{s})₂, —N(R′)S(O)₂R^{s}, —OC(O)N(R′)₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s};
s is 0-25;
R^{x} is -L-R′, —Si(R′)₃, or a hydroxyl protecting group;
L¹ is L;
each L is independently a covalent bond, or an optionally substituted, bivalent C₁₋₁₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
each -Cy- is independently an optionally substituted bivalent, 3-20 membered, monocyclic, bicyclic or polycyclic ring having 0-10 heteroatoms;
R′ is R, —OR, —C(O)R, —C(O)OR, —C(O)N(R)₂, or —S(O)₂R; and each R is independently —H, or an optionally substituted group selected from C₁₋₁₅ aliphatic, C₁₋₁₅ heteroaliphatic having 1-5 heteroatoms, C₆₋₁₄ aryl, C₆₋₁₄ aryl-C₁₋₁₅ aliphatic, C₆₋₁₄ aryl-C₁₋₁₅ heteroaliphatic having 1-5 heteroatoms, C₁₋₁₅ aliphatic-C₆₋₁₄ aryl, C₁₋₁₅ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₅ aliphatic, 5-14 membered heteroaryl having 1-10 heteroatoms-C₁₋₁₅ heteroaliphatic having 1-5 heteroatoms, C₁₋₁₅ aliphatic-5-14 membered heteroaryl having 1-10 heteroatoms, C₁₋₁₅ heteroaliphatic having 1-5 heteroatoms-5-14 membered heteroaryl having 1-10 heteroatoms, C₂₋₂₀ biaryl having 0-10 heteroatoms, and 3-20 membered heterocyclyl having 1-5 heteroatoms, or
two R groups are optionally and independently taken together to form a covalent bond or ═O, or:
two or more R groups on the same atom are optionally and independently taken together with the atom to form an optionally substituted, 3-20 membered, monocyclic, bicyclic or polycyclic ring having, in addition to the atom, 0-5 heteroatoms; or
two or more R groups on two or more atoms are optionally and independently taken together with their intervening atoms to form an optionally substituted, 3-20 membered, monocyclic, bicyclic or polycyclic ring having, in addition to the intervening atoms, 0-5 heteroatoms;
each heteroatom is independently selected nitrogen, oxygen and sulfur; and wherein:
(1) one of R¹ and R^{1a} is —H and the other is halogen: or
(2) R^{L} is —C(O)N(R^{s})₂, —C(O)N(R′)S(O)₂R^{s}, —C(O)N(R′)C(R′)₂C(O)N(R^{s})₂, —C(O)N(R′)C(R′)₂S(O)₂R^{s}, —C(O)N(R′)C(R′)₂S(O)₂N(R^{s})₂, —C(O)N(R′)C(R′)₂P(O)(R^{s})₂, —C(O)N(R′)C(R′)₂N(R′)C(O)N(R′)S(O)₂R^{s}, —C(O)N(R′)C(R)₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂OR^{s}, —N(R^{s})₂, —N(R′)C(O)R^{s}, —N(R′)C(S)R^{s}, —N(R′)C(NR′)R^{s}, —N(R′)C(O)OR^{s}, —N(R′)C(O)N(R^{s})₂, —N(R′)C(NR′)N(R^{s})₂, —N(R′)C(O)N(R′)S(O)₂R^{s}, —N(R′)C(S)N(R′)S(O)₂R^{s}, —N(R′)C(NR′)N(R′)S(O)₂R^{s}, —N(R′)C(O)C(O)N(R′)S(O)₂R^{s}, —N(R′)C(O)N(R′)S(O)₂N(R^{s})₂, —N(R′)S(O)₂R^{s}, —OC(O)N(R^{s})₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s}; or
(3) L¹ is —CH(CH₃)—(CH₂)ₘ—, wherein m is 4-6.

3. The compound of claim 2, wherein each of R¹ and R^{1a} is independently —H.

4-74. (canceled)

75. The compound of claim 2, wherein one of R¹ and R^{1a} is —H and the other is —F or —Cl.

76. The compound of claim 2, wherein each of R¹ and R^{1a} is —F.

77. The compound of claim 2, wherein the compound is a compound of a formula II-j or a salt thereof: 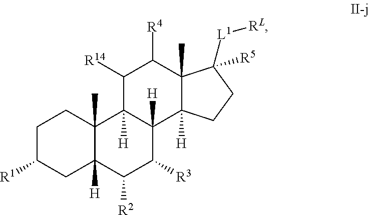
wherein each of R², R³, R⁴, R⁵, and R¹⁴ is independently R^{s}.

78. The compound of claim 77, wherein R^{L} is —N(R′)C(O)N(R′)S(O)₂R^{s}, —N(R′)C(S)N(R′)S(O)₂R^{s}, —N(R′)C(NR′)N(R′)S(O)₂R^{s}, —N(R′)C(O)C(O)N(R′)S(O)₂R^{s}, —N(R′)C(O)N(R′)S(O)₂N(R^{s})₂, —N(R′)S(O)₂R^{s}, —OC(O)N(R′)C(O)N(R′)S(O)₂R^{s}, or —OC(O)N(R′)S(O)₂R^{s}.

79. The compound of claim 77, wherein R¹ is —H.

80. The compound of claim 78, wherein R¹ is —H.

81. The compound of claim 80, wherein R² is ethyl.

82. The compound of claim 81, wherein R³ is —OH.

83. The compound of claim 2, wherein the compound is a compound of a formula II-p or a salt thereof: 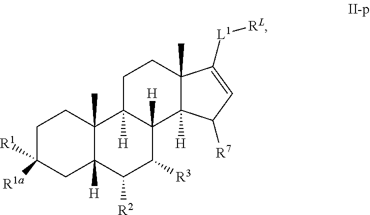
wherein each of R², R³, and R⁷ is independently R^{s}.

84. The compound of claim 2, wherein the compound is a compound of a formula II-s or a salt thereof: 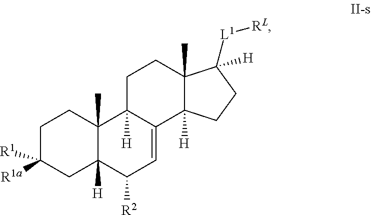
wherein R² is R^{s}.

85. The compound of claim 2, wherein the compound is 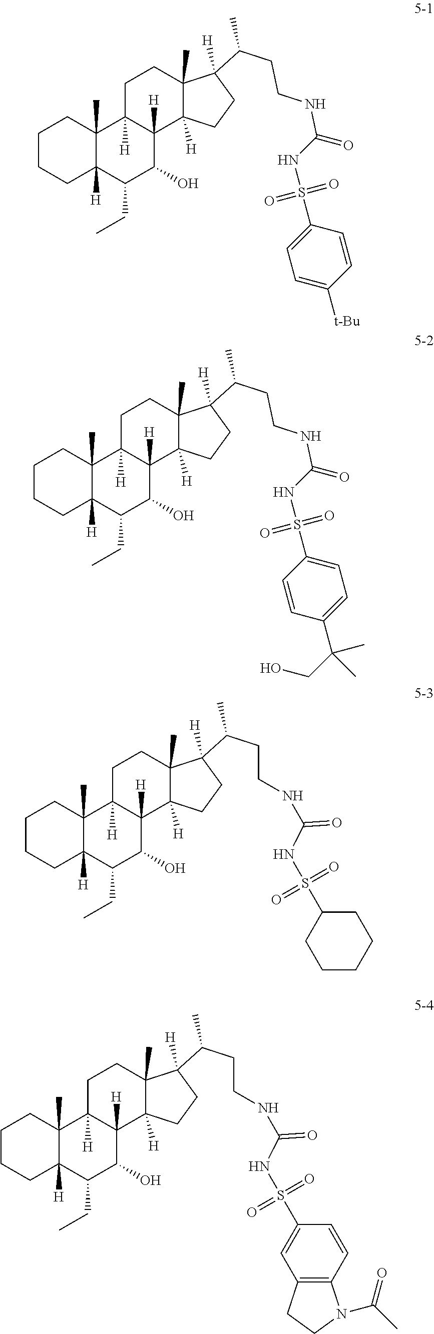 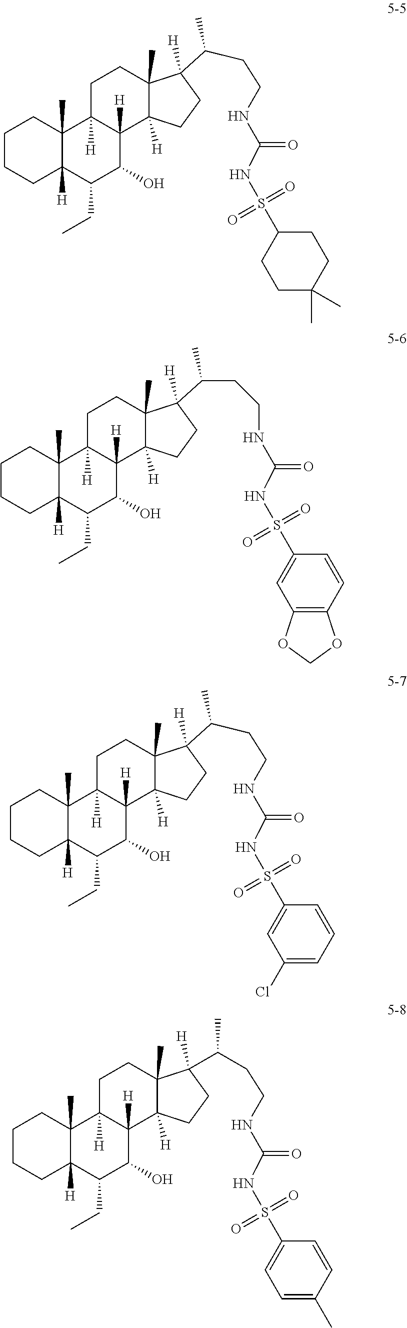 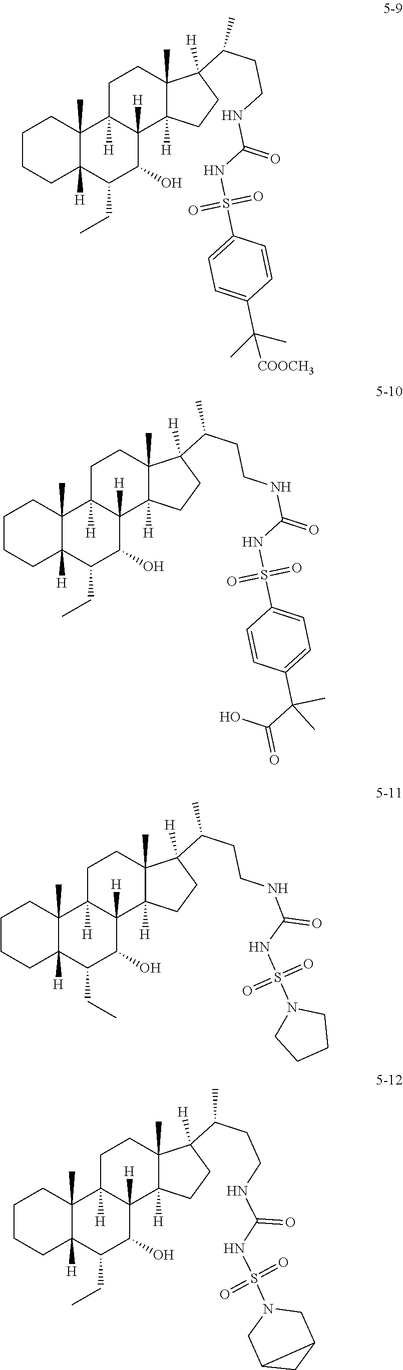 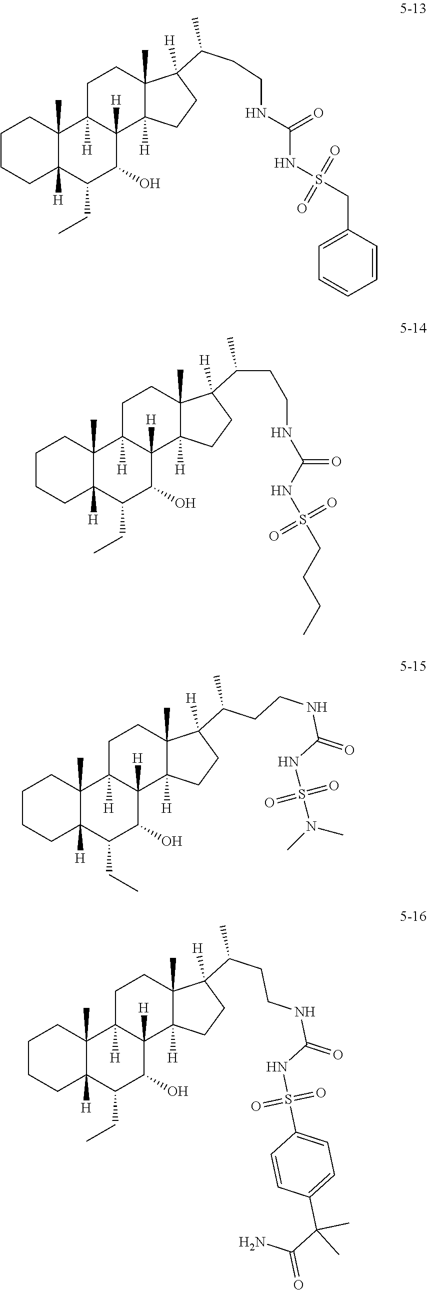 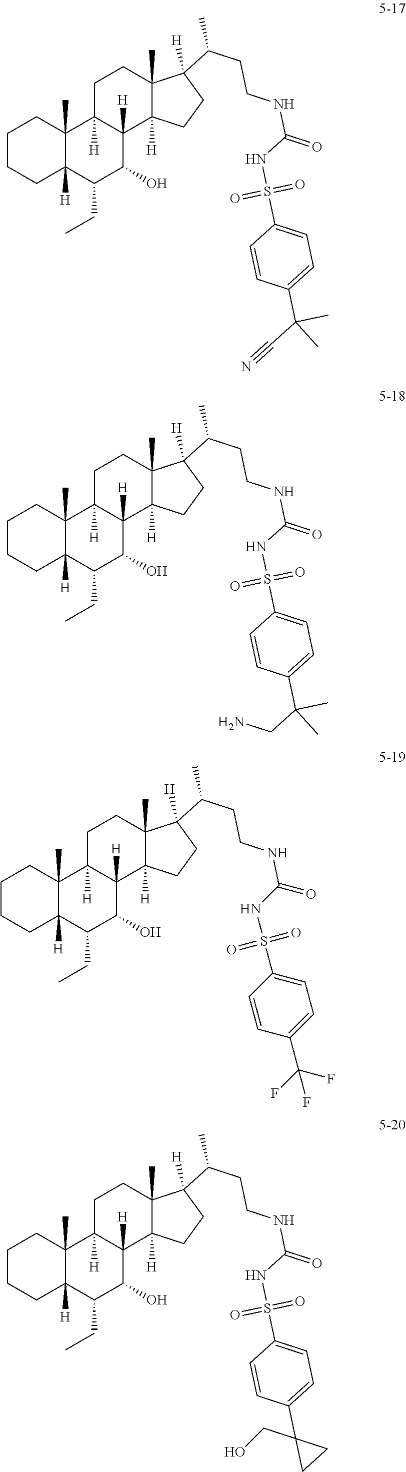 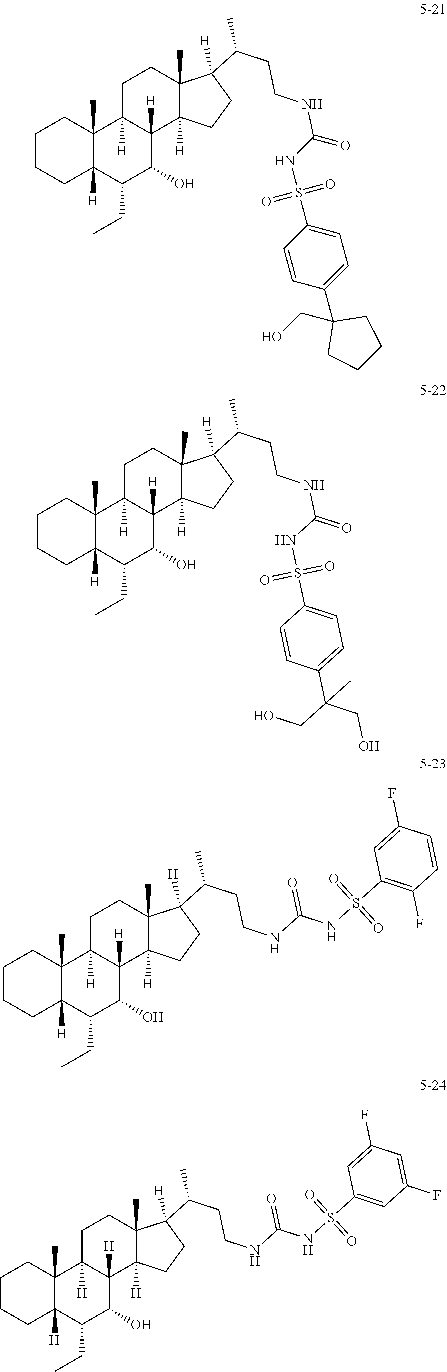 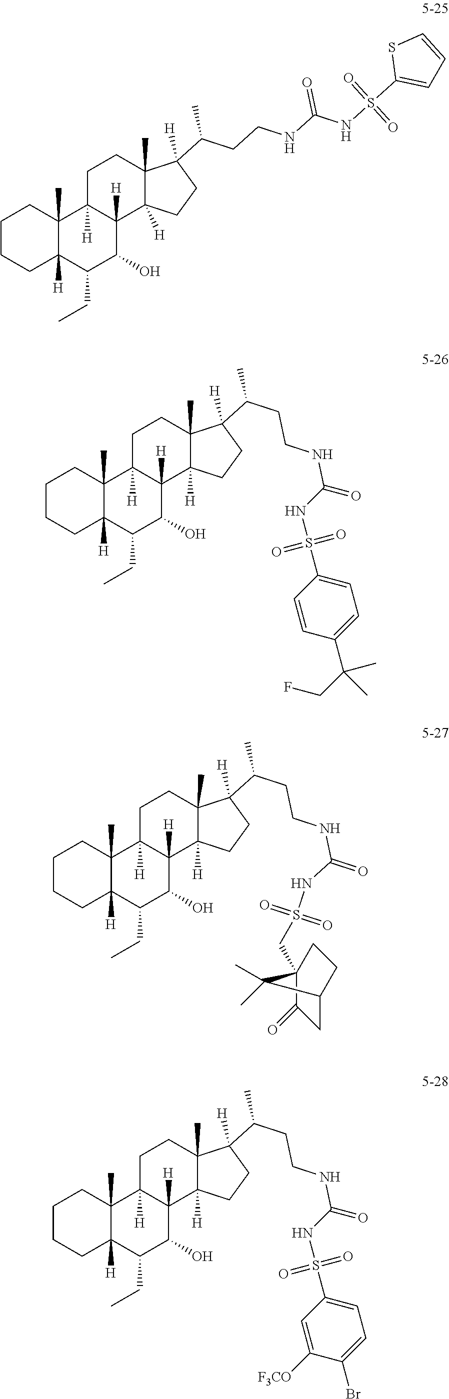 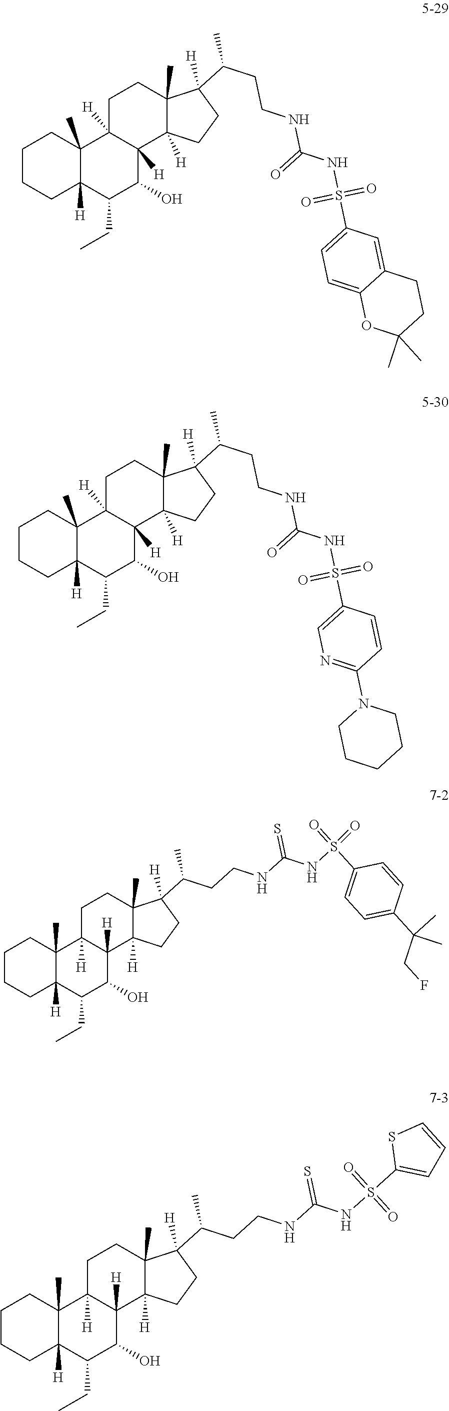 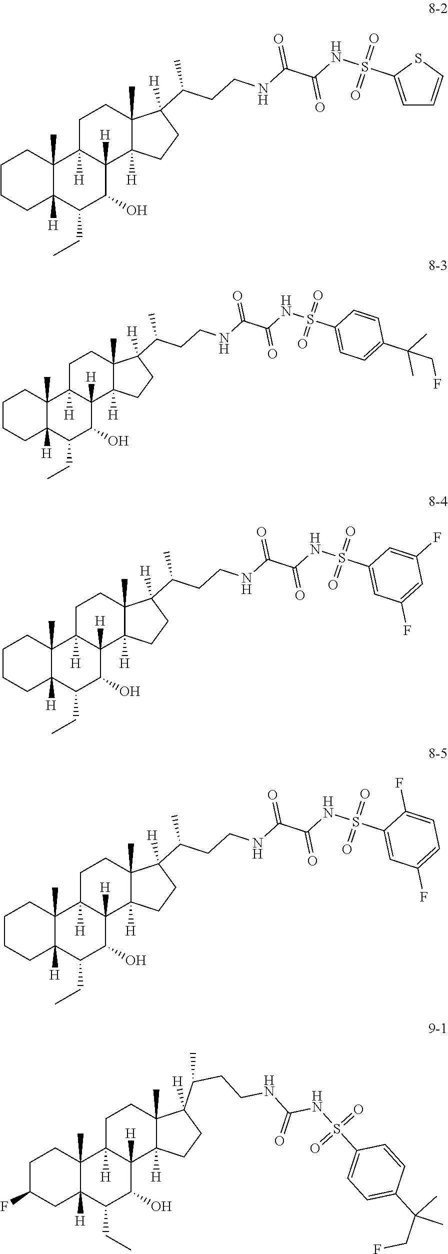 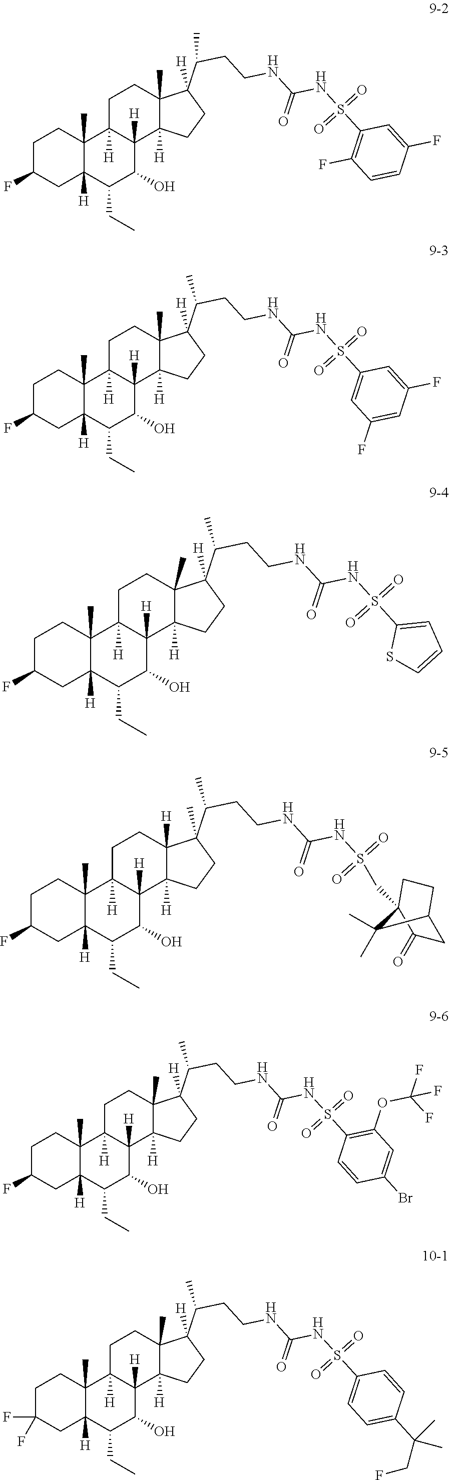 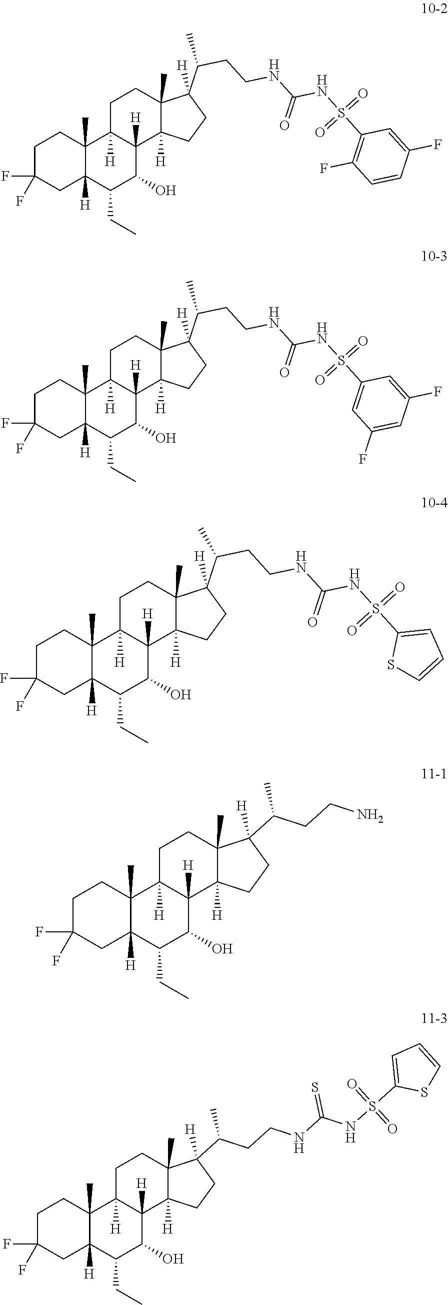 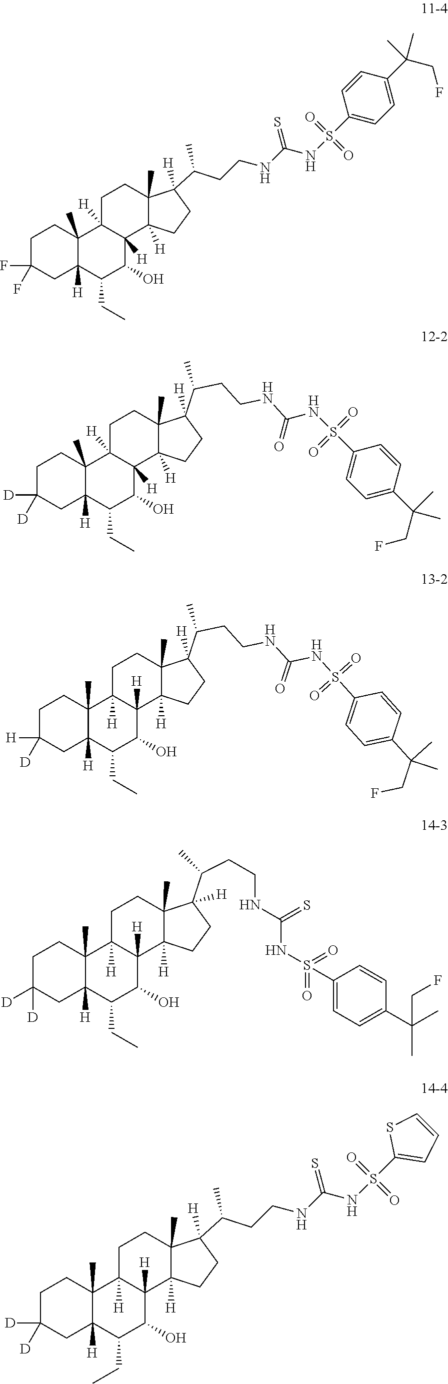 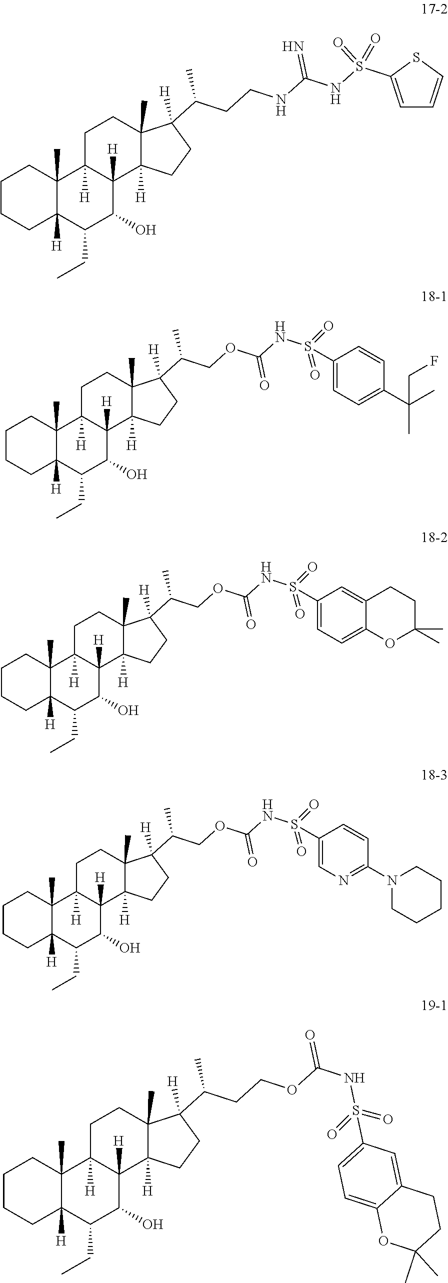 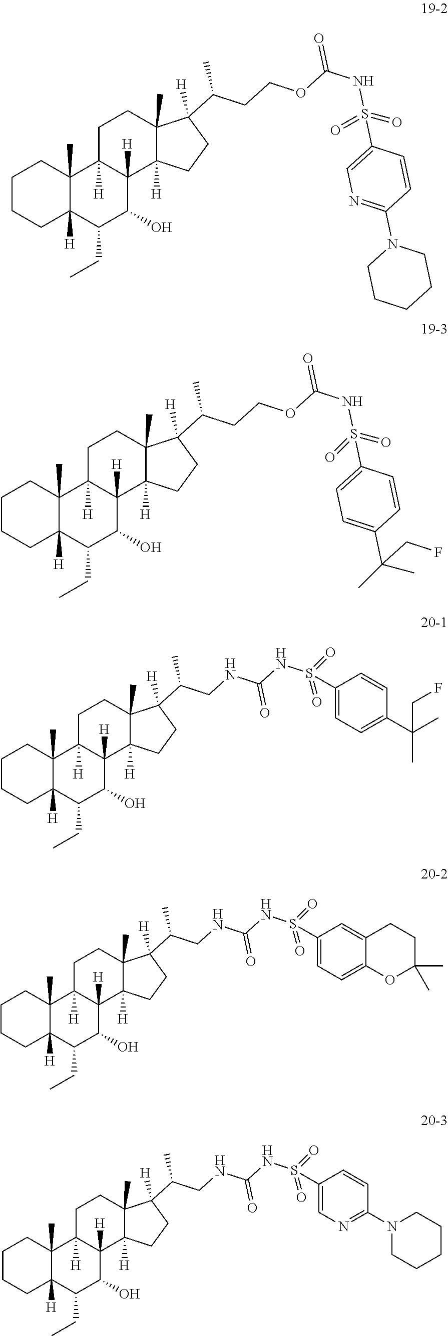 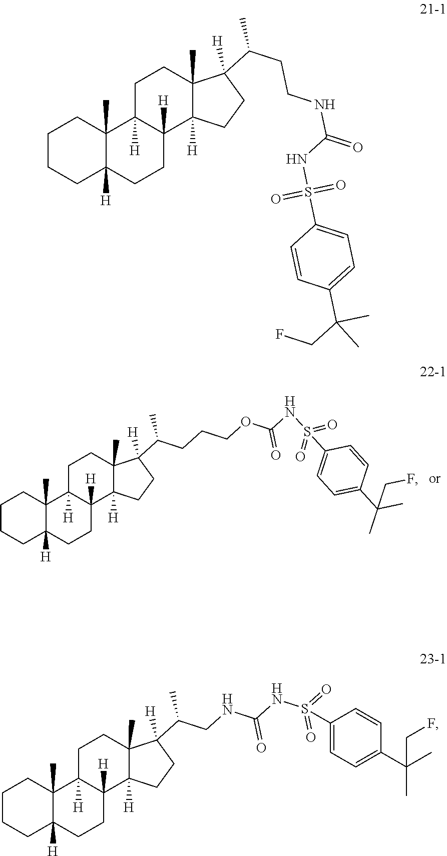
or a salt thereof.

86. A pharmaceutical composition, comprising a compound of claim 2 and pharmaceutically acceptable carrier.

87. A composition comprising a compound of claim 2, wherein one or more isotopes are enriched at one or more locations.

88. A method comprising:
assessing activation of FXR and/or TGR5 by a compound and a reference compound;
assessing activation of MRGPRX4 by the compound and the reference compound;
wherein the compound provides higher selectivity for the activation of FXR and/or TGR5 over MRGPRX4;
optionally wherein the compound comprises moiety A, or wherein the compound is a bile acid or a bile acid derivative, or a salt thereof, or wherein the compound is a compound of formula I or a salt thereof: 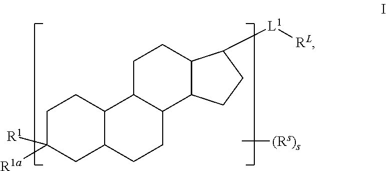
wherein:
each of R¹ and R^{1a} is independently —R^{s};
each R^{s} is independently —H, -L″-R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, —N(R′)₂, a protected hydroxyl group, or R^{s} is 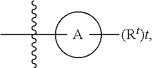
or two R^{s} attached to the same atom are taken together to form ═O or ═NR^{x};
t is 0-6;
each R is independently R′, halogen, —CN, —N₃, —OR′, —C(O)R′, —S(O)₂R′, —S(O)₂N(R′)₂, —SO₃R′, —OS(O)₂R′, —OP(O)(R′)₂, —OP(O)(OR′)₂, —P(O)(R′)₂, —PO(OR′)₂, —SR′, —C(O)N(R′)₂, or —N(R′)₂;
each Ring A is independently an optionally substituted 3-20 membered monocyclic, bicyclic or polycyclic ring having 0-10 heteroatoms;
each L″ is independently a covalent bond, or an optionally substituted, bivalent C₁₋₆ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, C(O)S—, C(O)O—, C(S)—, C(NR′), —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
R^{L} is R^{s}, —C(O)R^{s}, —C(O)OR^{s}, —C(O)N(R^{s})₂, —C(O)N(R′)S(O)₂R^{s}, —C(O)N(R′)C(R′)₂C(O)N(R^{s})₂, —C(O)N(R′)C(R′)₂S(O)₂R^{s}, —C(O)N(R′)C(R′)₂S(O)₂N(R^{s})₂, —C(O)N(R′)C(R′)₂P(O)(R^{s})₂, —C(O)N(R′)C(R′)₂N(R′)C(O)N(R′)S(O)₂R^{s}, —C(O)N(R′)C(R^{s})₃, —C(O)N(R′)C(R′)₂C(O)N(R′)S(O)₂R^{s}, —C(O)N(R′)S(O)₂N(R^{s})₂, —C(O)N(R′)C(NR′)N(R^{s})₂, —S(O)₂R^{s}, —S(O)₂N(R^{s})₂, —P(O)(R^{s})₂, —OS(O)₂R^{s}, —OS(O)₂₀R^{s}, —N(R^{s})₂, —N(R′)C(O)R^{s}, —N(R′)C(S)R^{s}, —N(R′)C(NR′)R^{s}, —N(R′)C(O)OR^{s}, —N(R′)C(O)N(R^{s})₂, —N(R′)C(NR′)N(R^{s})₂, —N(R′)C(O)N(R′)S(O)₂R′, —N(R′)C(S)N(R′)S(O)₂R^{s}, —N(R′)C(NR′)N(R′)S(O)₂R^{s}, —N(R′)C(O)C(O)N(R′)S(O)₂R′, —N(R′)C(O)N(R′)S(O)₂N(R^{s})₂, —N(R′)S(O)₂R^{s}, —OC(O)N(R′)₂, —OC(O)N(R′)C(O)N(R^{s})₂, —OC(O)N(R′)C(O)R^{s}, —OC(O)N(R′)C(O)N(R′)S(O)₂R^{s}, or —OC(O)N(R′)S(O)₂R^{s};
s is 0-25;
R^{x} is -L-R′, —Si(R′)₃, or a hydroxyl protecting group;
L¹ is L;
each L is independently a covalent bond, or an optionally substituted, bivalent C₁₋₁₅ aliphatic or heteroaliphatic group having 1-10 heteroatoms wherein one or more methylene units of the group are optionally and independently replaced with —C(R′)₂—, —CH═CH—, —C≡C—, -Cy-, —O—, —S—, —S—S—, —N(R′)—, —C(O)—, —C(O)S—, —C(O)O—, —C(S)—, —C(NR′)—, —C(O)N(R′)—, —C(NR′)N(R′)—, —C(S)N(R′)—, —N(R′)C(O)N(R′)—, —N(R′)C(NR′)N(R′)—, —N(R′)C(S)N(R′)—, —N(R′)C(O)O—, —N(R′)C(O)N(R′)S(O)₂—, —OC(O)N(R′)—, —OC(O)N(R′)S(O)₂—, —S(O)—, —S(O)₂—, —S(O)₂N(R′)—, —P(O)(OR′)—, or —P(O)(OR′)O—;
each -Cy- is independently an optionally substituted bivalent, 3-20 membered, monocyclic, bicyclic or polycyclic ring having 0-10 heteroatoms;
R′ is R, —OR, —C(O)R, —C(O)OR, —C(O)N(R)₂, or —S(O)₂R; and each R is independently —H, or an optionally substituted group selected from C₁₋₁₅ aliphatic, C₁₋₁₅ heteroaliphatic having 1-5 heteroatoms, C₆₋₁₄ aryl, C₆₋₁₄ aryl-C₁₋₁₅ aliphatic, C₆₋₁₄ aryl-C₁₋₁₅ heteroaliphatic having 1-5 heteroatoms, C₁₋₁₅ aliphatic-C₆₋₁₄ aryl, C₁₋₁₅ heteroaliphatic having 1-5 heteroatoms-C₆₋₁₄ aryl, 5-14 membered heteroaryl having 1-10 heteroatoms, 5-14 membered heteroaryl having 1-10 heteroatoms-C₁₋₁₅ aliphatic, 5-14 membered heteroaryl having 1-10 heteroatoms-C₁₋₁₅ heteroaliphatic having 1-5 heteroatoms, C₁₋₁₅ aliphatic-5-14 membered heteroaryl having 1-10 heteroatoms, C₁₋₁₅ heteroaliphatic having 1-5 heteroatoms-5-14 membered heteroaryl having 1-10 heteroatoms, C₂₋₂₀ biaryl having 0-10 heteroatoms, and 3-20 membered heterocyclyl having 1-5 heteroatoms, or
two R groups are optionally and independently taken together to form a covalent bond or ═O, or:
two or more R groups on the same atom are optionally and independently taken together with the atom to form an optionally substituted, 3-20 membered, monocyclic, bicyclic or polycyclic ring having, in addition to the atom, 0-5 heteroatoms; or
two or more R groups on two or more atoms are optionally and independently taken together with their intervening atoms to form an optionally substituted, 3-20 membered, monocyclic, bicyclic or polycyclic ring having, in addition to the intervening atoms, 0-5 heteroatoms.

89. A method selected from:
a) a method for activating FXR, comprising contacting FXR with a compound;
b) a method for activating FXR in a system, comprising administering or delivering to the system a compound;
c) a method for activating TGR5, comprising contacting TGR5 with a compound; and
d) a method for activating TGR5 in a system, comprising administering or delivering to the system a compound;
wherein in each of a) to d), the compound is a compound of claim 2.

90. A method for preventing or treating a condition, disorder or disease in a subject, comprising administering or delivering to a subject susceptible thereto or suffering therefrom an effective amount of a compound of claim 2.

91. The method of claim 90, wherein the condition, disease or disorder is nonalcoholic steatohepatitis.

92. A method selected from:
a) a method comprising:
contacting a compound comprising a leaving group and moiety A or a salt thereof with a reducing agent; and
producing a compound comprising moiety A but not the leaving group or a salt thereof, and
b) a method, comprising:
contacting a compound having the structure of HO—C(O)-L″-Cy-S(O)₂N(R′)₂ or a salt thereof with a reducing agent to provide a compound having the structure of HO—CH₂-L″-Cy-S(O)₂N(R′)₂ or a salt thereof, wherein each of L″, -Cy-, and R′ is independently as defined in claim 2.


SEQUENCE LISTING (2 sequences)

SEQ ID NO: 1 - DNA, 1431 bp, Homo sapiens
  source 1..1431: mol_type = genomic DNA
atgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgtatgtatacaggcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

SEQ ID NO: 2 - protein, 476 aa, Homo sapiens
MGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECMYTGLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ